(19) United States
(12) Patent Application Publication
(10) Pub. No.: US 2025/0326749 A1
(43) Pub. Date: Oct. 23, 2025

(54) COMPOUNDS, COMPOSITIONS, AND METHODS OF USING THEREOF

(71) Applicant: Sionna Therapeutics Inc., Waltham, MA (US)

(72) Inventors: Junkai Liao, Andover, MA (US); Mark Munson, Acton, MA (US); Zhongli Gao, Waltham, MA (US); Gregory Hurlbut, Boxborough, MA (US); John E. Macor, Washington Crossing, PA (US)

(21) Appl. No.: 18/873,248

(22) PCT Filed: Jun. 9, 2023
(86) PCT No.: PCT/US2023/068239

§ 371 Date: Dec. 9, 2024

Related U.S. Application Data
(60) Provisional application No. 63/350,970, filed on Jun. 10, 2022.

(51) Int. Cl. C07D 471/04; A61K 31/416; A61K 31/4245; A61K 31/4355; A61K 31/437; A61K 31/4439; A61K 31/444; A61K 31/454; A61K 31/4545; A61K 31/501; C07D 405/12; C07D 405/14; C07D 413/14; C07D 491/056; C07D 519/00

(52) U.S. Cl. CPC C07D 471/04; A61K 31/416; A61K 31/4245; A61K 31/4355; A61K 31/437; A61K 31/4439; A61K 31/444; A61K 31/454; A61K 31/4545; A61K 31/501; C07D 405/12; C07D 405/14; C07D 413/14; C07D 491/056; C07D 519/00

(57) ABSTRACT

The present disclosure relates to heterocyclic compounds, pharmaceutically acceptable salts thereof, and pharmaceutical preparations thereof. Also described herein are compositions and the use of such compounds in methods of treating diseases and conditions mediated by deficient CFTR activity, in particular cystic fibrosis.

### CROSS-REFERENCE TO RELATED APPLICATIONS

This application claims the benefit of, and priority to, U.S. Provisional Application No. 63/350,970, filed on Jun. 10, 2022, the contents of which is incorporated herein by reference in its entirety.

### BACKGROUND

Cystic fibrosis (CF), an autosomal recessive disorder, is caused by functional deficiency of the CAMP-activated plasma membrane chloride channel, cystic fibrosis transmembrane conductance regulator (CFTR), which results in pulmonary and other complications. The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). CFTR, a member of the ATP binding cassette (ABC) superfamily is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate, and thiocyanate into and out of the cell. CFTR may have a regulatory role over other electrolyte channels, including the epithelial sodium channel ENaC.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure.

While more than 1,900 mutations have been identified in the CFTR gene, a detailed understanding of how each CFTR mutation may impact channel function is known for only a few. (Derichs, European Respiratory Review, 22:127, 58-65 (2013)). The most frequent CFTR mutation is the in-frame deletion of phenylalanine at residue 508 (ΔF508) in the first nucleotide binding domain (NBD1). Over 70% of cystic fibrosis patients have a deletion at residue 508 in at least one CFTR allele. The loss of this key phenylalanine renders NBD1 conformationally unstable at physiological temperature and compromises the integrity of the interdomain interface between NBD1 and CFTR's second transmembrane domain (ICL4). The ΔF508 mutation causes production of misfolded CFTR protein which, rather than traffic to the plasma membrane, is instead retained in the endoplasmic reticulum and targeted for degradation by the ubiquitin-proteasome system.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation. In the lung, the loss of CFTR-function leads to numerous physiological effects downstream of altered anion conductance that result in the dysfunction of additional organs such as the pancreas, intestine and gall bladder.

By studying the mechanistic aspects of CFTR misfolding and corrections, small molecules have been identified as CF modulators, that can act as stabilizers.

Despite the identification of compounds that modulate CFTR, there is no cure for this fatal disease and identification of new compounds and new methods of therapy are needed as well as new methods for treating or lessening the severity of cystic fibrosis and other CFTR mediated conditions and diseases in a patient.

### SUMMARY

The present disclosure includes a compound of Formula (I) or (II)

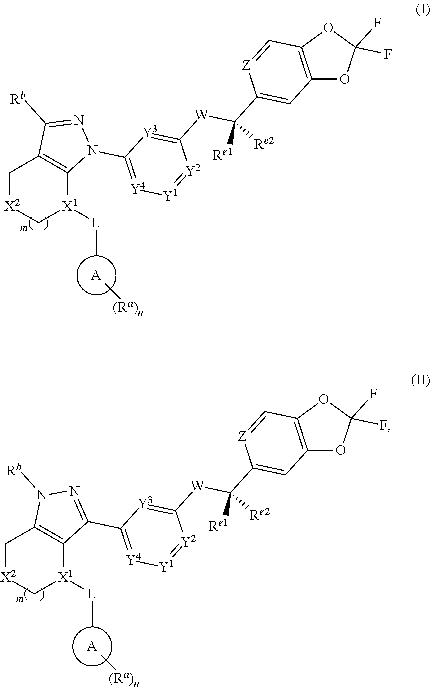

or a pharmaceutically acceptable salt thereof Additionally, the present disclosure includes, among other things, pharmaceutical compositions, methods of using and methods of making a compound of Formula (I) or (II).

### DETAILED DESCRIPTION

In some embodiments, the present disclosure includes a compound of Formula (I) or (II)

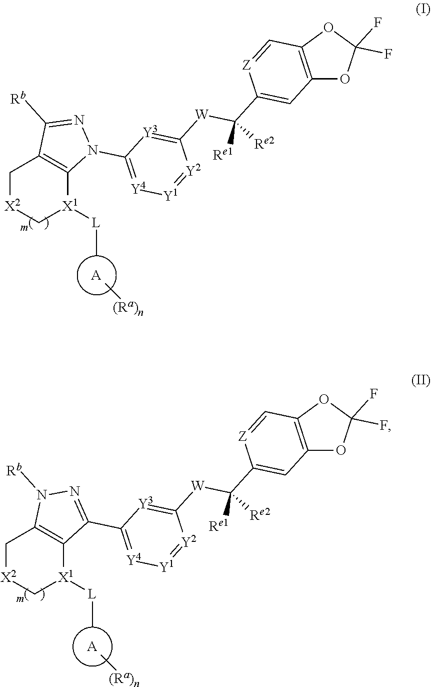

or a pharmaceutically acceptable salt thereof
A is selected from the group consisting of optionally substituted 3-10 membered carbocyclyl, optionally substituted 3-10 membered heterocyclyl, optionally substituted phenyl, and optionally substituted 5-10 membered heteroaryl;
L is selected from the group consisting of a bond, —O—, —S—, —S(O)₂—, —CH₂—, —C(O)—, —N(H)—, and —N(H)C(O)—;
W is selected from the group consisting of —O—, —S—, —S(O)—, —S(O)₂—, —N(H)—, and optionally substituted 3-6 membered heterocyclylene;
X¹ is N or C(H);
X² is selected from the group consisting of —CH₂—, —N(H)—, and —N(1-3 alkyl)-;
Y¹ is selected from the group consisting of —C(H)═, —C(F)═, and —N═;
Y² is selected from the group consisting of —C(H)═, —C(F)═, and —N═;
Y³ is selected from the group consisting of —C(H)═, —C(F)═, and —N═;
Y⁴ is selected from the group consisting of —C(H)═, —C(F)═, and —N═;
Z is selected from the group consisting of —C(H)═, —C(F)═, and —N═;
each R^{a} is independently selected from the group consisting of halogen, optionally substituted C₁-C₃ aliphatic, optionally substituted 5-7 membered heterocyclyl, optionally substituted 5-6 membered heteroaryl, and —COOH;
R^{b} is selected from the group consisting of —CN, optionally substituted C₁-C₆ aliphatic, optionally substituted C₁-C₆ haloaliphatic, and optionally substituted 3-7 membered heterocyclyl;
R^{e1} is selected from the group consisting of hydrogen, C₁-C₅-alkyl, and C₃-C₅-cycloalkyl;
R^{e2} is selected from the group consisting of hydrogen, C₁-C₅-alkyl, and C₃-C₅-cycloalkyl;
   wherein R^{e1} and R^{e2}, together with the atom on which they are attached, are optionally taken together to form an optionally substituted 3-6 membered carbocyclyl ring;
m is 0-2; and
n is 0-5.

In some embodiments, the present disclosure includes a compound of Formula (I):

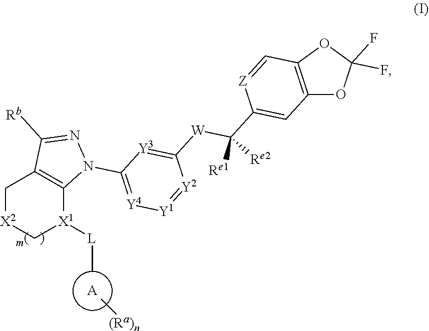

or a pharmaceutically acceptable salt thereof
A is selected from the group consisting of optionally substituted 3-10 membered carbocyclyl, optionally substituted 3-10 membered heterocyclyl, optionally substituted aryl, and optionally substituted 5-10 membered heteroaryl;
L is selected from the group consisting of a bond, —O—, —S—, —S(O)₂—, —CH₂—, —C(O)—, and —N(H)—;
X is N or C;
Y¹ is selected from the group consisting of —C(H)═, —C(F), and —N═;
Y² is selected from the group consisting of —C(H)═, —C(F), and —N═;
Y³ is selected from the group consisting of —C(H)═, —C(F), and —N═;
Y⁴ is selected from the group consisting of —C(H)═, —C(F), and —N═;
Z is selected from the group consisting of —C(H)═, —C(F)═, and —N═;
each R^{a} is independently selected from the group consisting of halogen, optionally substituted C₁-C₃ aliphatic, and —COOH;
R^{e1} is selected from the group consisting of hydrogen, C₁-C₅-alkyl, and C₃-C₅-cycloalkyl; R^{e2} is selected from the group consisting of hydrogen, C₁-C₅-alkyl, and C₃-C₅-cycloalkyl;
m is 0-2; and
n is 0-5.

In some embodiments the present disclosure includes a compound that is represented by Formula (I-a) or (II-a)

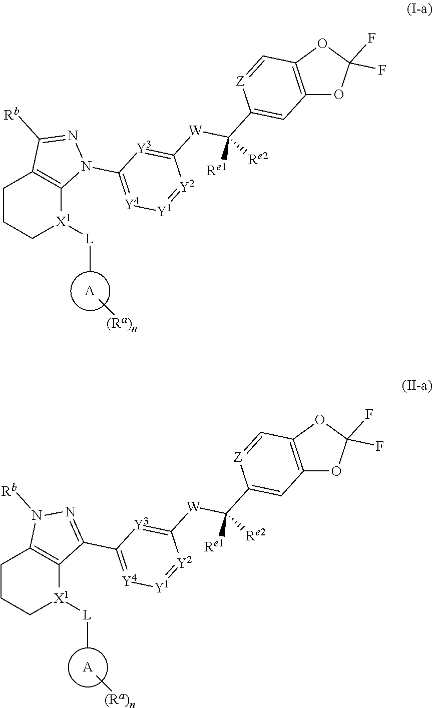

or a pharmaceutically acceptable salt thereof.

In some embodiments, the present disclosure includes a compound of Formula (I-a′):

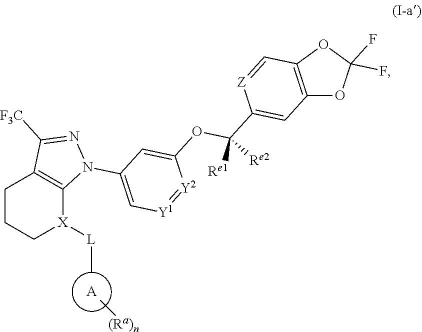

or a pharmaceutically acceptable salt thereof.

In some embodiments, the present disclosure includes a compound is represented by Formula (I-a1) or (I-a2)

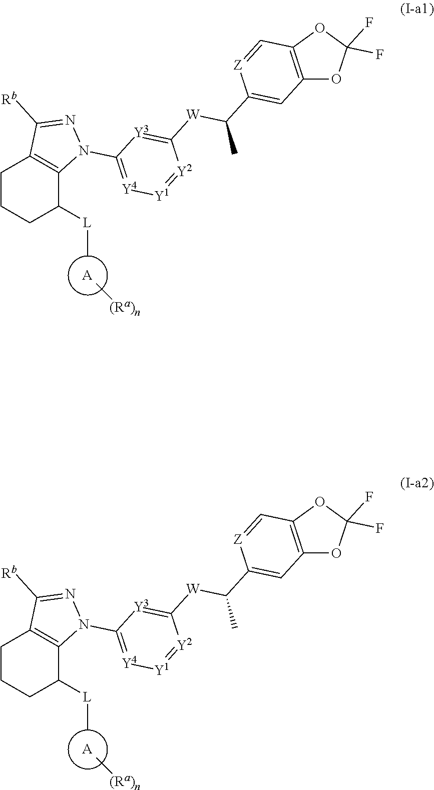

or a pharmaceutically acceptable salt thereof.

In some embodiments, the present disclosure includes a compound of Formula (I-b1) or (I-b2)

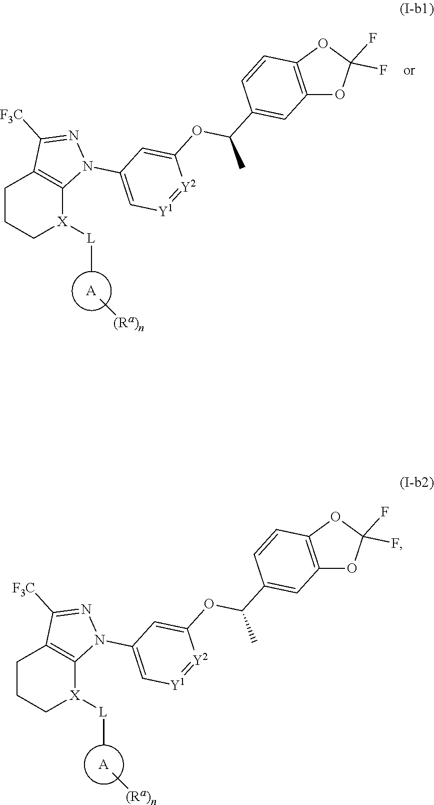

or a pharmaceutically acceptable salt thereof.

### A

In some embodiments, A is selected from the group consisting of optionally substituted 3-10 membered carbocyclyl, optionally substituted 3-10 membered heterocyclyl, optionally substituted phenyl, and optionally substituted 5-10 membered heteroaryl. In some embodiments, A is selected from the group consisting of optionally substituted 3-10 membered carbocyclyl, optionally substituted 3-10 membered heterocyclyl, optionally substituted aryl, and optionally substituted 5-10 membered heteroaryl. In some embodiments, A is selected from the group consisting of optionally substituted 5-8 membered carbocyclyl, optionally substituted 5-7 membered heterocyclyl, and optionally substituted phenyl. In some embodiments, A is optionally substituted 5-8 membered carbocyclyl. In some embodiments, A is optionally substituted 5-7 membered heterocyclyl. In some embodiments, A is optionally substituted phenyl. In some embodiments, A is selected from the group consisting of optionally substituted cyclohexyl, optionally substituted bicyclo[1.1.1]pentyl, optionally substituted bicyclo[2.2.2]octyl, optionally substituted piperidinyl, and optionally substituted phenyl. In some embodiments, A is selected from the group consisting of

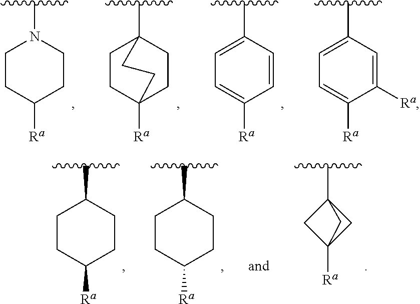

In some embodiments, A is selected from the group consisting of

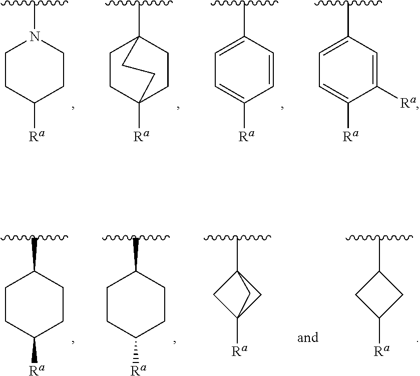

### L

In some embodiments, L is selected from the group consisting of a bond, —O—, —S—, —S(O)₂—, —CH₂—, —C(O)—, —N(H)—, and —N(H)C(O)—. In some embodiments, L is selected from the group consisting of a bond, —O—, —S—, —S(O)₂—, —CH₂—, —C(O)—, and —N(H)—. In some embodiments, L is selected from the group consisting of —S(O)₂—, —CH₂—, and —C(O)—. In some embodiments, L is —O—. In some embodiments, L is —S—. In some embodiments, L is —S(O)₂—. In some embodiments, L is —CH₂—. In some embodiments, L is —C(O)—. In some embodiments, L is —N(H)—.

X¹ and X²

In some embodiments, X¹ is N or C(H). In some embodiments, X¹ is N. In some embodiments, X¹ is C(H).

In some embodiments, X² is selected from the group consisting of —CH₂—, —N(H)—, and —N(C₁₋₃ alkyl)-. In some embodiments X² is —CH₂—. In some embodiments, X² is —N(H)—. In some embodiments X² is —N(C₁₋₃ alkyl)-. In some embodiments, —N(Me)-.

### Y¹

In some embodiments, Y¹ is selected from the group consisting of —C(H)═, —C(F), and —N═. In some embodiments, Y¹ is —C(H)═. In some embodiments, Y¹ is —C(F)═. In some embodiments, Y¹ is —N═.

### Y²

In some embodiments, Y² is selected from the group consisting of —C(H)═, —C(F), and —N═. In some embodiments, Y² is —C(H)═. In some embodiments, Y² is —C(F)═. In some embodiments, Y² is —N═.

### Y³

In some embodiments, Y³ is selected from the group consisting of —C(H)═, —C(F), and —N═. In some embodiments, Y³ is —C(H)—. In some embodiments, Y³ is —C(F)═. In some embodiments, Y³ is —N═.

### Y⁴

In some embodiments, Y⁴ is selected from the group consisting of —C(H)═, —C(F), and —N═. In some embodiments, Y⁴ is —C(H)═. In some embodiments, Y⁴ is —C(F)═. In some embodiments, Y⁴ is —N═.

### Z

In some embodiments, Z is selected from the group consisting of —C(H)═, —C(F), and —N═. In some embodiments, Z is —C(H)═. In some embodiments, Z is —C(F)═. In some embodiments, Z is —N═.

### R^{a}

In some embodiments, each R^{a} is independently selected from the group consisting of halogen, optionally substituted C₁-C₃ aliphatic, optionally substituted 5-7 membered heterocyclyl, optionally substituted 5-6 membered heteroaryl, and —COOH. In some embodiments, each R^{a} is independently selected from the group consisting of halogen, optionally substituted C₁-C₃ aliphatic, and —COOH. In some embodiments, each R^{a} is independently selected from the group consisting of optionally substituted C₁-C₃ aliphatic, or —COOH. In some embodiments, each R^{a} is independently selected from optionally substituted C₁-C₃ aliphatic. In some embodiments, each R^{a} is independently selected from optionally substituted halogen. In some embodiments, each R^{a} is independently selected from the group consisting of methyl and —COOH. In some embodiments, R^{a} is methyl. In some embodiments, R^{a} is —COOH. In some embodiments, R^{a} is optionally substituted 5-6 membered heterocyclyl.

In some embodiments, R^{a} is

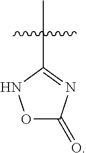

### R^{e1}

In some embodiments, R^{e1} is selected from the group consisting of hydrogen, C₁-C₅-alkyl, and C₃-C₅-cycloalkyl. In some embodiments, R^{e1} is hydrogen. In some embodiments, R^{e1} is C₁-C₅-alkyl. In some embodiments, R^{e1} is C₁-C₃-alkyl. In some embodiments, R^{e1} is methyl.

### R^{e2}

In some embodiments, R^{e2} is selected from the group consisting of hydrogen, C₁-C₅-alkyl, and C₃-C₅-cycloalkyl. In some embodiments, R^{e2} is hydrogen. In some embodiments, R^{e2} is C₁-C₅-alkyl. In some embodiments, R^{e2} is C₁-C₃-alkyl. In some embodiments, R^{e2} is methyl.

### M

In some embodiments, m is 0-2. In some embodiments, m is 0. In some embodiments, m is 1. In some embodiments, m is 2.

### N

In some embodiments, n is 0-5. In some embodiments, n is 0-2. In some embodiments, n is 0. In some embodiments, n is 1. In some embodiments, n is 2. In some embodiments, n is 3. In some embodiments, n is 4. In some embodiments, n is 5.

The present disclosure includes a compound of Table 1.

TABLE 1

| Ex. No. | Structure | IUPAC name |
|---|---|---|
| 1 | 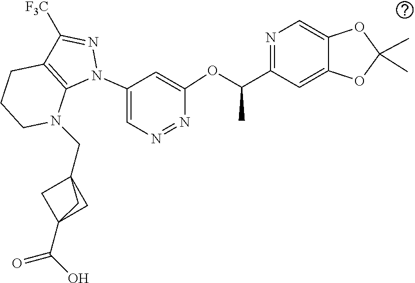 [SMILES: C[C@@H](Oc1cc(-n2nc(C(F)(F)F)c3c2N(CC24CC(C(=O)O)(C2)C4)CCC3)cnn1)c1cc2c(cn1)OC(C)(C)O2] | 3-[[1-[6-[(1R)-1-(2,2-difluoro- [1,3]dioxolo[4,5-c]pyridin-6- yl)ethoxy]pyridazin-4-yl]-3- (trifluoromethyl)-5,6-dihydro-4H- pyrazolo[3,4-b]pyridin-7- yl]methyl]bicyclo[1.1.1]pentane-1- carboxylic acid |
| 2 | 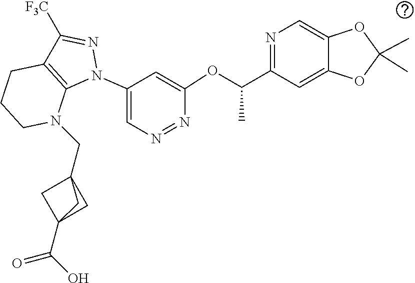 [SMILES: C[C@H](Oc1cc(-n2nc(C(F)(F)F)c3c2N(CC24CC(C(=O)O)(C2)C4)CCC3)cnn1)c1cc2c(cn1)OC(C)(C)O2] | 3-[[1-[6-[(1S)-1-(2,2-difluoro- [1,3]dioxolo[4,5-c]pyridin-6- yl)ethoxy]pyridazin-4-yl]-3- (trifluoromethyl)-5,6-dihydro-4H- pyrazolo[3,4-b]pyridin-7- yl]methyl]bicyclo[1.1.1]pentane-1- carboxylic acid |
| 3 | 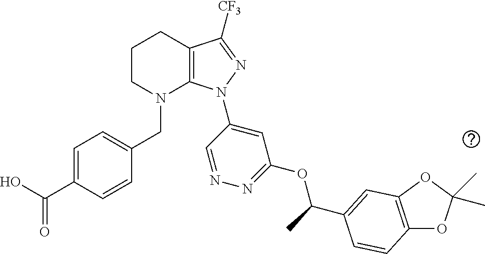 [SMILES: C[C@@H](Oc1cc(-n2nc(C(F)(F)F)c3c2N(Cc2ccc(C(=O)O)cc2)CCC3)cnn1)c1ccc2c(c1)OC(C)(C)O2] | -[[1-[6-[(1R)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]benzoic acid |
| 4 | 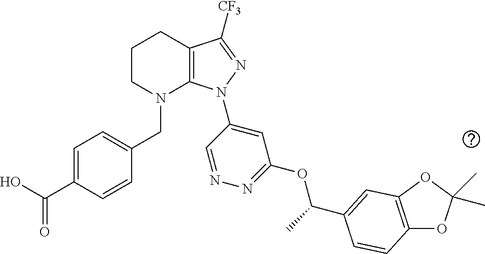 [SMILES: C[C@H](Oc1cc(-n2nc(C(F)(F)F)c3c2N(Cc2ccc(C(=O)O)cc2)CCC3)cnn1)c1ccc2c(c1)OC(C)(C)O2] | 4-[[1-[6-[(1S)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]benzoic acid |
| 5 | 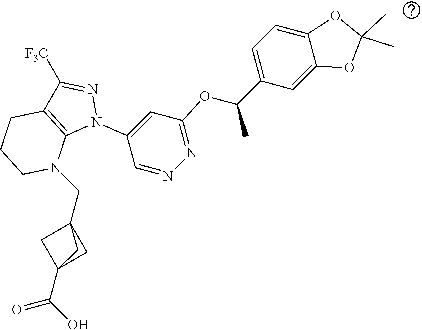 [SMILES: C[C@@H](Oc1cc(-n2nc(C(F)(F)F)c3c2N(CC24CC(C(=O)O)(C2)C4)CCC3)cnn1)c1ccc2c(c1)OC(C)(C)O2] | 3-[[1-[6-[(1R)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]bicyclo[1.1.1]pentane-1- carboxylic acid |
| 6 | 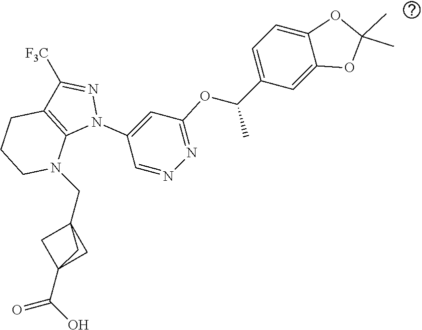 [SMILES: C[C@H](Oc1cc(-n2nc(C(F)(F)F)c3c2N(CC24CC(C(=O)O)(C2)C4)CCC3)cnn1)c1ccc2c(c1)OC(C)(C)O2] | 3-[[1-[6-[(1S)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]bicyclo[1.1.1]pentane-1- carboxylic acid |
| 7 | 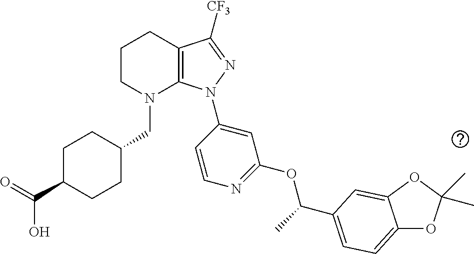 [SMILES: C[C@H](Oc1cc(-n2nc(C(F)(F)F)c3c2N(C[C@H]2CC[C@H](C(=O)O)CC2)CCC3)ccn1)c1ccc2c(c1)OC(C)(C)O2] | Trans-4-[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]cyclohexanecarboxylic acid |
| 8 | 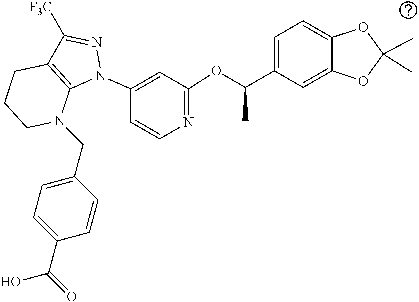 [SMILES: C[C@@H](Oc1cc(-n2nc(C(F)(F)F)c3c2N(Cc2ccc(C(=O)O)cc2)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]benzoic acid |
| 9 | 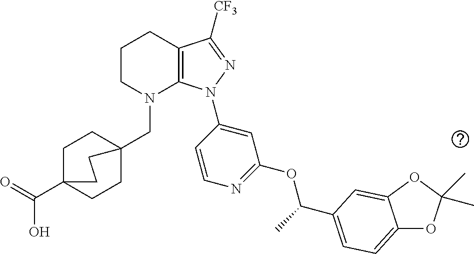 [SMILES: C[C@H](Oc1cc(-n2nc(C(F)(F)F)c3c2N(CC24CCC(C(=O)O)(CC2)CC4)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]bicyclo[2.2.2]octane-1- carboxylic acid |
| 10 | 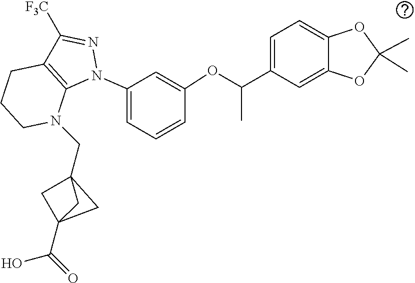 [SMILES: CC(Oc1cccc(-n2nc(C(F)(F)F)c3c2N(CC24CC(C(=O)O)(C2)C4)CCC3)c1)c1ccc2c(c1)OC(C)(C)O2] | 3-[[1-[3-[1-(2,2-difluoro-1,3- benzodioxol-5-yl)ethoxy]phenyl]-3- (trifluoromethyl)-5,6-dihydro-4H- pyrazolo[3,4-b]pyridine-7- yl]methyl]bicyclo[1.1.1]pentane-1- carboxylic acid |
| 11 | 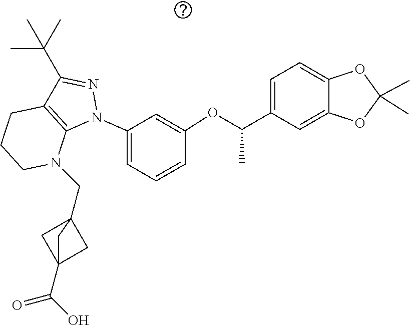 [SMILES: C[C@H](Oc1cccc(-n2nc(C(C)(C)C)c3c2N(CC24CC(C(=O)O)(C2)C4)CCC3)c1)c1ccc2c(c1)OC(C)(C)O2] | 3-[[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- yl]methyl]bicyclo[1.1.1]pentane-1- carboxylic acid |
| 12 | 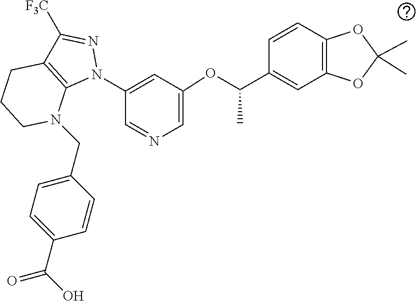 [SMILES: C[C@H](Oc1cncc(-n2nc(C(F)(F)F)c3c2N(Cc2ccc(C(=O)O)cc2)CCC3)c1)c1ccc2c(c1)OC(C)(C)O2] | 4-[1-[5-[(1S)-1-(2,2-difluoro-1,3- benzodioxol-5-yl)ethoxy]-3-pyridyl]-3- (trifluoromethyl)- 5,6-dihydro-4H-pyrazolo[3,4-b]pyridine- 7-yl]methyl]benzoic acid |
| 13 | 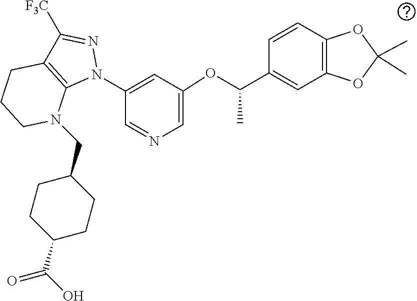 [SMILES: C[C@H](Oc1cncc(-n2nc(C(F)(F)F)c3c2N(C[C@H]2CC[C@H](C(=O)O)CC2)CCC3)c1)c1ccc2c(c1)OC(C)(C)O2] | Trans-4-[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- yl]methyl]cyclohexanecarboxylic acid |
| 14 | 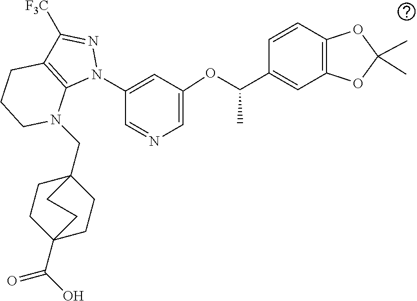 [SMILES: C[C@H](Oc1cncc(-n2nc(C(F)(F)F)c3c2N(CC24CCC(C(=O)O)(CC2)CC4)CCC3)c1)c1ccc2c(c1)OC(C)(C)O2] | 4-[[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- yl]methyl]bicyclo[2.2.2]octane-1- carboxylic acid |
| 15 | 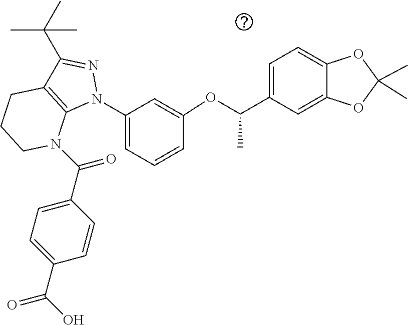 [SMILES: C[C@H](Oc1cccc(-n2nc(C(C)(C)C)c3c2N(C(=O)c2ccc(C(=O)O)cc2)CCC3)c1)c1ccc2c(c1)OC(C)(C)O2] | 4-[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- carbonyl]benzoic acid |
| 16 | 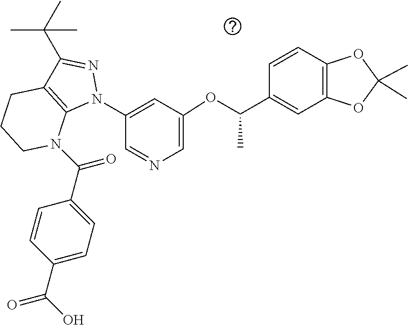 [SMILES: C[C@H](Oc1cncc(-n2nc(C(C)(C)C)c3c2N(C(=O)c2ccc(C(=O)O)cc2)CCC3)c1)c1ccc2c(c1)OC(C)(C)O2] | 4-[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- carbonyl]benzoic acid |
| 17 | 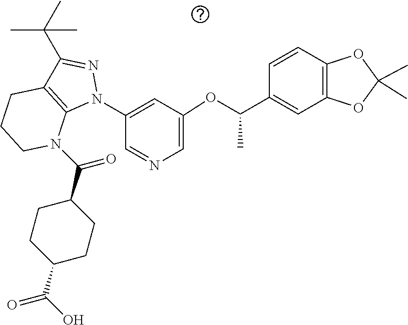 [SMILES: C[C@H](Oc1cncc(-n2nc(C(C)(C)C)c3c2N(C(=O)[C@H]2CC[C@H](C(=O)O)CC2)CCC3)c1)c1ccc2c(c1)OC(C)(C)O2] | 4-[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- carbonyl]cyclohexanecarboxylic acid |
| 18 | 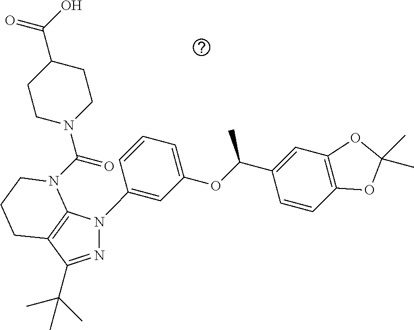 [SMILES: C[C@H](Oc1cccc(-n2nc(C(C)(C)C)c3c2N(C(=O)N2CCC(C(=O)O)CC2)CCC3)c1)c1ccc2c(c1)OC(C)(C)O2] | 1-[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- carbonyl]piperidine-4-carboxylic acid |
| 19 | 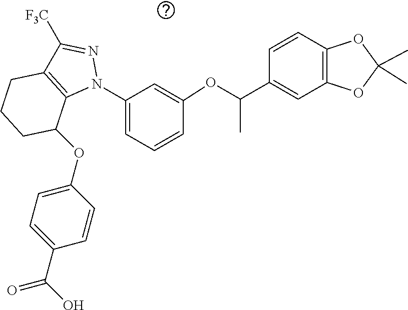 [SMILES: CC(Oc1cccc(-n2nc(C(F)(F)F)c3c2C(Oc2ccc(C(=O)O)cc2)CCC3)c1)c1ccc2c(c1)OC(C)(C)O2] | 4-[[1-[3-[1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 20 | 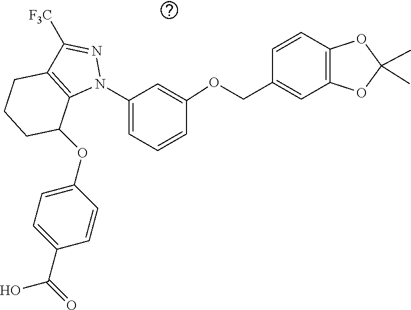 [SMILES: CC1(C)Oc2ccc(COc3cccc(-n4nc(C(F)(F)F)c5c4C(Oc4ccc(C(=O)O)cc4)CCC5)c3)cc2O1] | 4-[[1-[3-[(2,2-difluoro-1,3-benzodioxol- 5-yl)methoxy]phenyl]-3- (trifluoromethyl)-4,5,6,7- tetrahydroindazol-7-yl]oxy]benzoic acid |
| 21 | 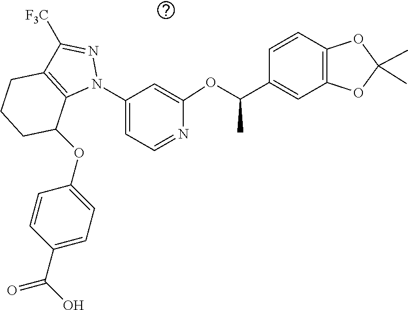 [SMILES: C[C@@H](Oc1cc(-n2nc(C(F)(F)F)c3c2C(Oc2ccc(C(=O)O)cc2)CCC3)ccn1)c1ccc2c(c1)OC(C)(C)O2] | 4-[[1-[2-[(1R)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 22 | 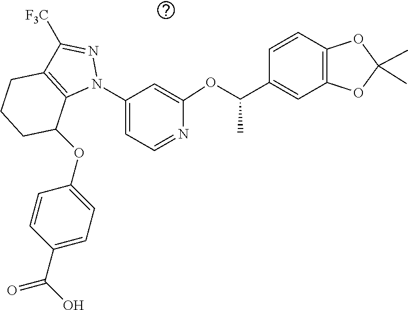 [SMILES: C[C@H](Oc1cc(-n2nc(C(F)(F)F)c3c2C(Oc2ccc(C(=O)O)cc2)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 23 | 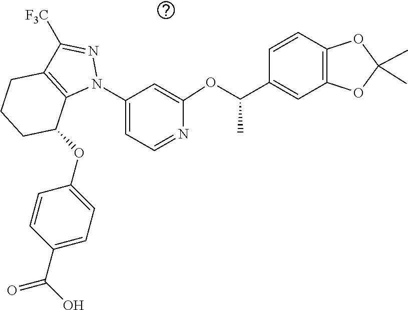 [SMILES: C[C@H](Oc1cc(-n2nc(C(F)(F)F)c3c2[C@H](Oc2ccc(C(=O)O)cc2)CCC3)ccn1)c1ccc2c(c1)OC(C)(C)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 24 | 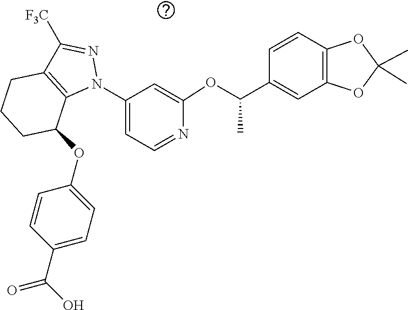 [SMILES: C[C@H](Oc1cc(-n2nc(C(F)(F)F)c3c2[C@@H](Oc2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 25 | 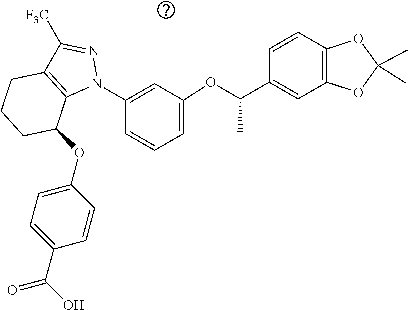 [SMILES: C[C@H](Oc1cccc(-n2nc(C(F)(F)F)c3c2[C@@H](Oc2ccc(C(=O)O)cc2)CCC3)c1)c1ccc2c(c1)OC(C)(C)O2] | 4-[[(7S)-1-[3-[(1S)-1-(2,2-difluoro-1,3- benzodioxol-5-yl)ethoxyphenyl]-3- (trifluoromethyl)-4,5,6,7- tetrahydroindazol-7-yl]oxy]benzoic acid |
| 26 | 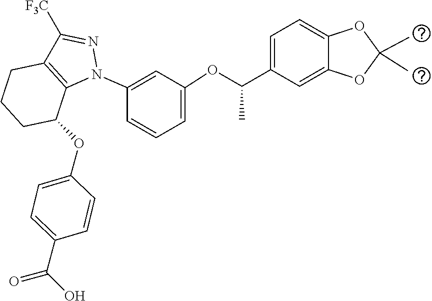 [SMILES: C[C@H](Oc1cccc(-n2nc(C(F)(F)F)c3c2[C@H](Oc2ccc(C(=O)O)cc2)CCC3)c1)c1ccc2c(c1)OC(C)(C)O2] | 4-[[(7R)-1-[3-[(1S)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 27 | 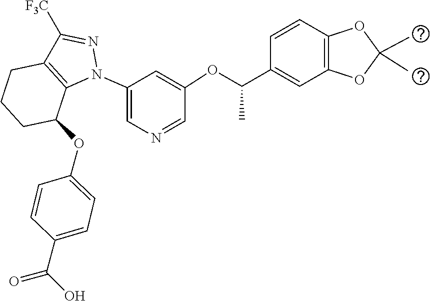 [SMILES: C[C@H](Oc1cncc(-n2nc(C(F)(F)F)c3c2[C@@H](Oc2ccc(C(=O)O)cc2)CCC3)c1)c1ccc2c(c1)OC(C)(C)O2] | 4-[[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 28 | 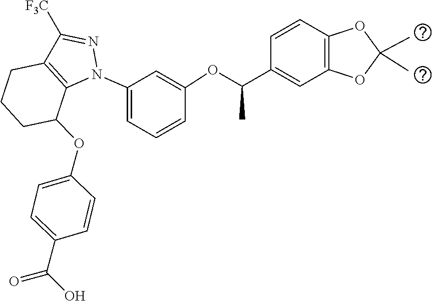 [SMILES: C[C@@H](Oc1cccc(-n2nc(C(F)(F)F)c3c2C(Oc2ccc(C(=O)O)cc2)CCC3)c1)c1ccc2c(c1)OC(C)(C)O2] | 4-[[1-[3-[(1R)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 29 | 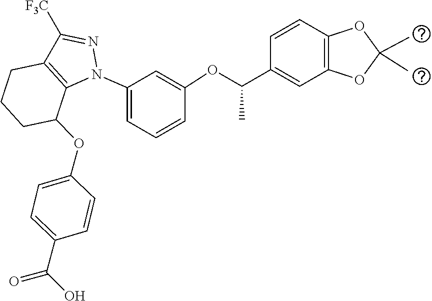 [SMILES: C[C@H](Oc1cccc(-n2nc(C(F)(F)F)c3c2C(Oc2ccc(C(=O)O)cc2)CCC3)c1)c1ccc2c(c1)OC(C)(C)O2] | 4-[[1-[3-[(1S)-1-(2,2-difluoro-1,3- benzodioxol-5-yl)ethoxyphenyl]-3- (trifluoromethyl)-4,5,6,7- tetrahydroindazol-7-yl]oxy]benzoic acid |
| 30 | 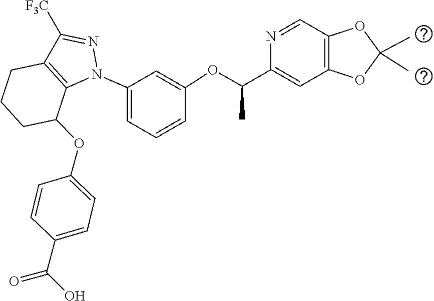 [SMILES: C[C@@H](Oc1cccc(-n2nc(C(F)(F)F)c3c2C(Oc2ccc(C(=O)O)cc2)CCC3)c1)c1cc2c(cn1)OC(C)(C)O2] | 4-[[1-[3-[(1R)-1-(2,2-difluoro- [1,3]dioxolo[4,5-c]pyridine-6- yl)ethoxy]phenyl]-3-(trifluoromethyl)- 4,5,6,7-tetrahydroindazol-7- yl]oxy]benzoic acid |
| 31 | 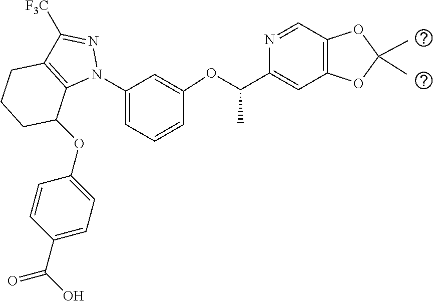 [SMILES: C[C@H](Oc1cccc(-n2nc(C(F)(F)F)c3c2C(Oc2ccc(C(=O)O)cc2)CCC3)c1)c1cc2c(cn1)OC(C)(C)O2] | 4-[[1-[3-[(1S)-1-(2,2-difluoro- [1,3]dioxolo[4,5-c]pyridine-6- yl)ethoxyphenyl]-3-(trifluoromethyl)- 4,5,6,7-tetrahydroindazol-7- yl]oxy]benzoic acid |
| 32 | 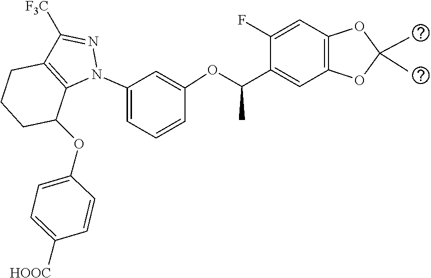 [SMILES: C[C@@H](Oc1cccc(-n2nc(C(F)(F)F)c3c2C(Oc2ccc(C(=O)O)cc2)CCC3)c1)c1cc2c(cc1F)OC(C)(C)O2] | 4-[1-[3-[(1R)-1-(2,2,6-trifluoro-1,3- benzodioxol-5-yl)ethoxy]phenyl]-3- (trifluoromethyl)-4,5,6,7- tetrahydroindazol-7-yl]oxy]benzoic acid |
| 33 | 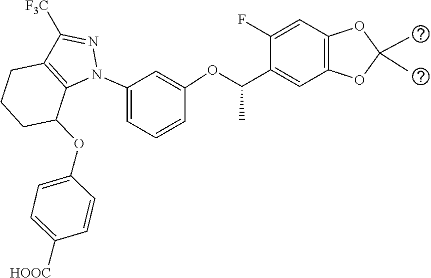 [SMILES: C[C@H](Oc1cccc(-n2nc(C(F)(F)F)c3c2C(Oc2ccc(C(=O)O)cc2)CCC3)c1)c1cc2c(cc1F)OC(C)(C)O2] | 4-[[1-[3-[(1S)-1-(2,2,6-trifluoro-1,3- benzodioxol-5-yl)ethoxyphenyl]-3- (trifluoromethyl)-4,5,6,7- tetrahydroindazol-7-yl]oxy]benzoic acid |
| 34 | 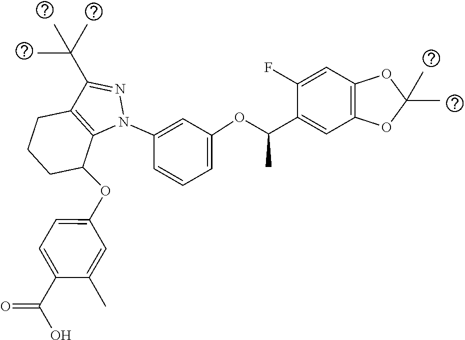 [SMILES: Cc1cc(OC2CCCc3c(C(C)(C)C)nn(-c4cccc(O[C@H](C)c5cc6c(cc5F)OC(C)(C)O6)c4)c32)ccc1C(=O)O] | 2-methyl-4-[[1-[3-[(1R)-1-(2,2,6- trifluoro-1,3-benzodioxol-5- yl)ethoxy]phenyl]-3-(trifluoromethyl)- 4,5,6,7-tetrahydroindazol-7- yl]oxy]benzoic acid |
| 35 | 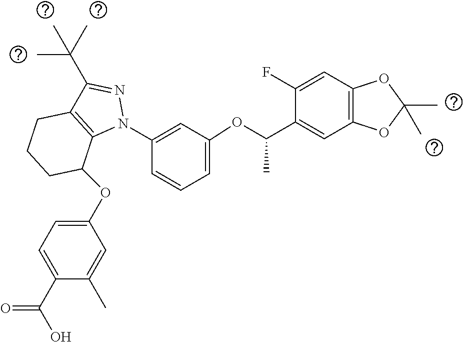 [SMILES: Cc1cc(OC2CCCc3c(C(C)(C)C)nn(-c4cccc(O[C@@H](C)c5cc6c(cc5F)OC(C)(C)O6)c4)c32)ccc1C(=O)O] | 2-methyl-4-[[1-[3-[(1S)-1-(2,2,6- trifluoro-1,3-benzodioxol-5- yl)ethoxy]phenyl]-3-(trifluoromethyl)- 4,5,6,7-tetrahydroindazol-7- yl]oxy]benzoic acid |
| 36 | 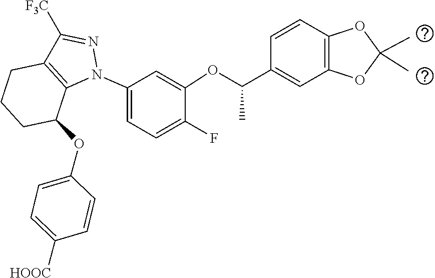 [SMILES: C[C@H](Oc1cc(-n2nc(C(F)(F)F)c3c2[C@@H](Oc2ccc(C(=O)O)cc2)CCC3)ccc1F)c1ccc2c(c1)OC(C)(C)O2] | 4-[[(7S)-1-[3-[(1S)-1-(2,2-difluoro-1,3- benzodioxol-5-yl)ethoxy]-4-fluoro- phenyl]-3-(trifluoromethyl)-4,5,6,7- tetrahydroindazol-7-yl]oxy]benzoic acid |
| 36a | 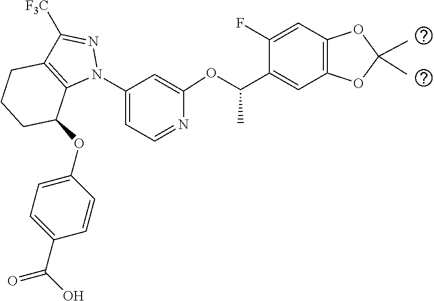 [SMILES: C[C@H](Oc1cc(-n2nc(C(F)(F)F)c3c2[C@@H](Oc2ccc(C(=O)O)cc2)CCC3)ccn1)c1cc2c(cc1F)OC(C)(C)O2] | 4-[[(7S)-1-[2-[(1S)-1-(2,2,6-trifluoro- 1,3-benzodioxol-5-yl)ethoxy]-4- pyridyl]-3-(trifluoromethyl)-4,5,6,7- tetrahydroindazol-7-yl]oxy]benzoic acid |
| 36b | 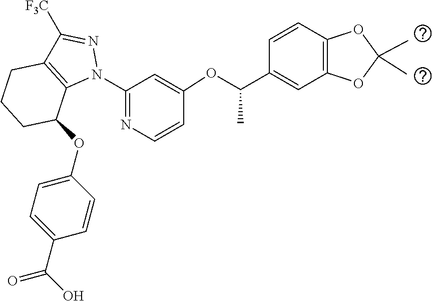 [SMILES: C[C@H](Oc1ccnc(-n2nc(C(F)(F)F)c3c2[C@@H](Oc2ccc(C(=O)O)cc2)CCC3)c1)c1ccc2c(c1)OC(C)(C)O2] | 4-[[(7S)-1-[4-[(1S)-1-(2,2-difluoro-1,3- benzodioxol-5-yl)ethoxy]-2-pyridyl]-3- (trifluoromethyl)-4,5,6,7- tetrahydroindazol-7-yl]oxy]benzoic acid |
| 36c | 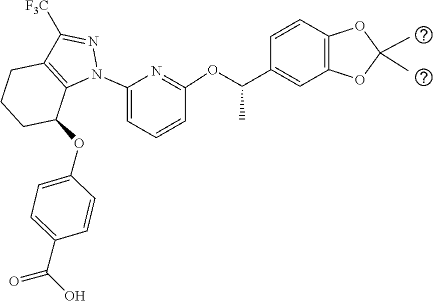 [SMILES: C[C@H](Oc1cccc(-n2nc(C(F)(F)F)c3c2[C@@H](Oc2ccc(C(=O)O)cc2)CCC3)n1)c1ccc2c(c1)OC(C)(C)O2] | 4-[[(7S)-1-[6-[(1S)-1-(2,2-difluoro-1,3- benzodioxol-5-yl)ethoxy]-2-pyridyl]-3- (trifluoromethyl)-4,5,6,7- tetrahydroindazol-7-yl]oxy]benzoic acid |
| 36d | 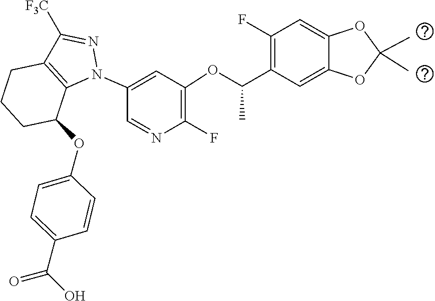 [SMILES: C[C@H](Oc1cc(-n2nc(C(F)(F)F)c3c2[C@@H](Oc2ccc(C(=O)O)cc2)CCC3)cnc1F)c1cc2c(cc1F)OC(C)(C)O2] | 4-[[(7S)-1-[6-fluoro-5-[(1S)-1-(2,2,6- trifluoro-1,3-benzodioxol-5-yl)ethoxy]- 3-pyridyl]-3-(trifluoromethyl)-4,5,6,7- tetrahydroindazol-7-yl]oxy]benzoic acid |
| 36e | 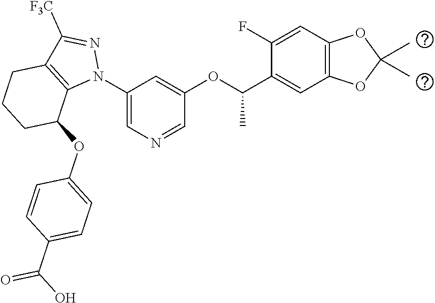 [SMILES: C[C@H](Oc1cncc(-n2nc(C(F)(F)F)c3c2[C@@H](Oc2ccc(C(=O)O)cc2)CCC3)c1)c1cc2c(cc1F)OC(C)(C)O2] | 4-[[(7S)-1-[5-[(1S)-1-(2,2,6-trifluoro- 1,3-benzodioxol-5-yl)ethoxy]-3- pyridyl]-3-(trifluoromethyl)-4,5,6,7- tetrahydroindazol-7-yl]oxy]benzoic acid |
| 36f | 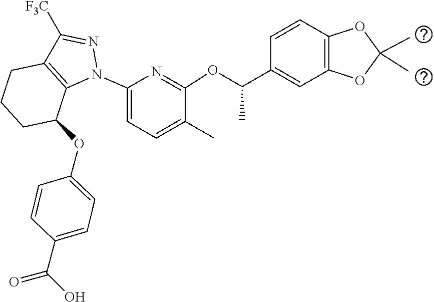 [SMILES: Cc1ccc(-n2nc(C(F)(F)F)c3c2[C@@H](Oc2ccc(C(=O)O)cc2)CCC3)nc1O[C@@H](C)c1ccc2c(c1)OC(C)(C)O2] | 4-[[(7S)-1-[6-[(1S)-1-(2,2-difluoro-1,3- benzodioxol-5-yl)ethoxy]-5-fluoro-2- pyridyl]-3-(trifluoromethyl)-4,5,6,7- tetrahydroindazol-7-yl]oxy]benzoic acid |
| 36g | 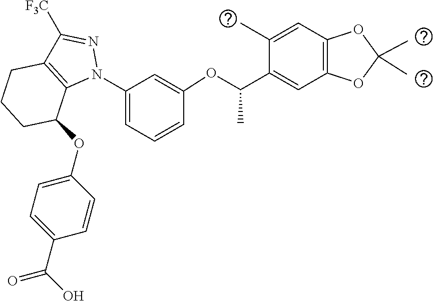 [SMILES: Cc1cc2c(cc1[C@H](C)Oc1cccc(-n3nc(C(F)(F)F)c4c3[C@@H](Oc3ccc(C(=O)O)cc3)CCC4)c1)OC(C)(C)O2] | 4-[[(7S)-1-[3-[(1S)-1-(2,2,6-trifluoro- 1,3-benzodioxol-5-yl)ethoxy]phenyl]-3- (trifluoromethyl)-4,5,6,7- tetrahydroindazol-7-yl]oxy]benzoic acid |
| 36h | 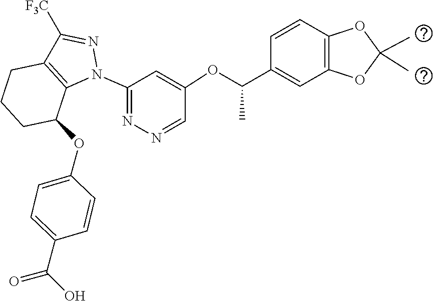 [SMILES: C[C@H](Oc1cnnc(-n2nc(C(F)(F)F)c3c2[C@@H](Oc2ccc(C(=O)O)cc2)CCC3)c1)c1ccc2c(c1)OC(C)(C)O2] | 4-[[(7S)-1-[5-[(1S)-1-(2,2-difluoro-1,3- benzodioxol-5-yl)ethoxy]pyridazin-3- yl]-3-(trifluoromethyl)-4,5,6,7- tetrahydroindazol-7-yl]oxy]benzoic acid |
| 36i | 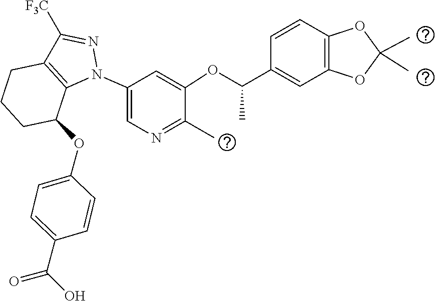 [SMILES: Cc1ncc(-n2nc(C(F)(F)F)c3c2[C@@H](Oc2ccc(C(=O)O)cc2)CCC3)cc1O[C@@H](C)c1ccc2c(c1)OC(C)(C)O2] | 4-[[(7S)-1-[4-[(1S)-1-(2,2-difluoro-1,3- benzodioxol-5-yl)ethoxy]-5-fluoro-2- pyridyl]-3-(trifluoromethyl)-4,5,6,7- tetrahydroindazol-7-yl]oxy]benzoic acid |
| 36j | 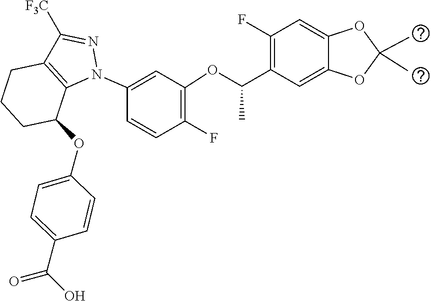 [SMILES: C[C@H](Oc1cc(-n2nc(C(F)(F)F)c3c2[C@@H](Oc2ccc(C(=O)O)cc2)CCC3)ccc1F)c1cc2c(cc1F)OC(C)(C)O2] | 4-[[(7S)-1-[4-fluoro-3-[(1S)-1-(2,2,6- trifluoro-1,3-benzodioxol-5- yl)ethoxy]phenyl]-3-(trifluoromethyl)- 4,5,6,7-tetrahydroindazol-7- yl]oxy]benzoic acid |
| 36k | 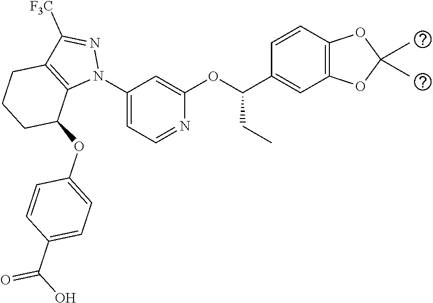 [SMILES: CC[C@H](Oc1cc(-n2nc(C(F)(F)F)c3c2[C@@H](Oc2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)propoxy]-4-pyridyl]-3- (trifluoromethyl)-4,5,6,7- tetrahydroindazol-7-yl]oxy]benzoic acid |
| 361 | 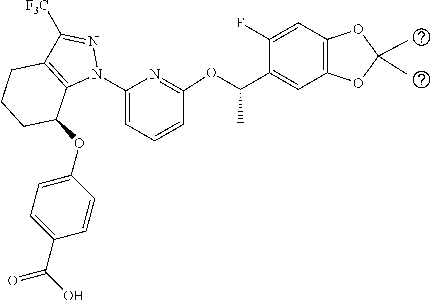 [SMILES: C[C@H](Oc1cccc(-n2nc(C(F)(F)F)c3c2[C@@H](Oc2ccc(C(=O)O)cc2)CCC3)n1)c1cc2c(cc1F)OC(C)(C)O2] | 4-[[(7S)-1-[6-[(1S)-1-(2,2,6-trifluoro- 1,3-benzodioxol-5-yl)ethoxy]-2- pyridyl]-3-(trifluoromethyl)-4,5,6,7- tetrahydroindazol-7-yl]oxy]benzoic acid |
| 36m | 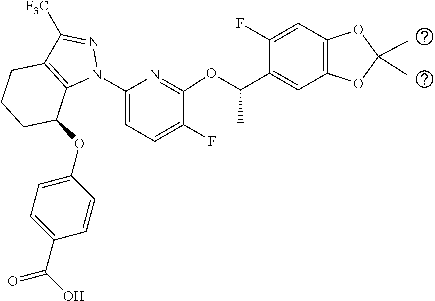 [SMILES: C[C@H](Oc1nc(-n2nc(C(F)(F)F)c3c2[C@@H](Oc2ccc(C(=O)O)cc2)CCC3)ccc1F)c1cc2c(cc1F)OC(C)(C)O2] | 4-[[(7S)-1-[5-fluoro-6-[(1S)-1-(2,2,6- trifluoro-1,3-benzodioxol-5-yl)ethoxy]- 2-pyridyl]-3-(trifluoromethyl)-4,5,6,7- tetrahydroindazol-7-yl]oxy]benzoic acid |
| 37 | 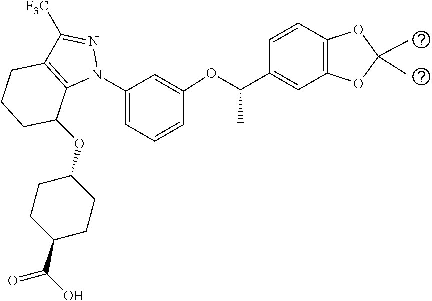 [SMILES: C[C@H](Oc1cccc(-n2nc(C(F)(F)F)c3c2C(O[C@H]2CC[C@H](C(=O)O)CC2)CCC3)c1)c1ccc2c(c1)OC(C)(C)O2] | Trans-4-[[1-[3-[(1S)-1-(2,2-difluoro-1,3- benzodioxol-5-yl)ethoxy]phenyl]-3- (trifluoromethyl)-4,5,6,7- tetrahydroindazol-7- yl]oxy]cyclohexanecarboxylic acid |
| 38 | 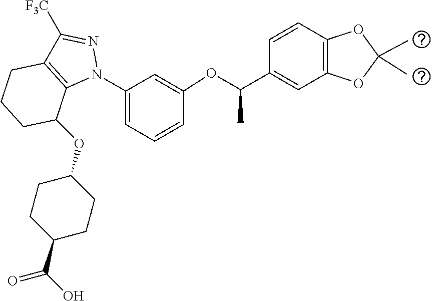 [SMILES: C[C@@H](Oc1cccc(-n2nc(C(F)(F)F)c3c2C(O[C@H]2CC[C@H](C(=O)O)CC2)CCC3)c1)c1ccc2c(c1)OC(C)(C)O2] | Trans-4-[1-[3-[(1R)-1-(2,2-difluoro-1,3- benzodioxol-5-yl)ethoxy]phenyl]-3- (trifluoromethyl)-4,5,6,7- tetrahydroindazol-7- yl]oxy]cyclohexanecarboxylic acid |
| 39 | 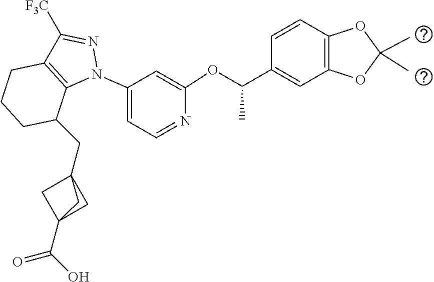 [SMILES: C[C@H](Oc1cc(-n2nc(C(F)(F)F)c3c2C(CC24CC(C(=O)O)(C2)C4)CCC3)ccn1)c1ccc2c(c1)OC(C)(C)O2] | 3-[[1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 40 | 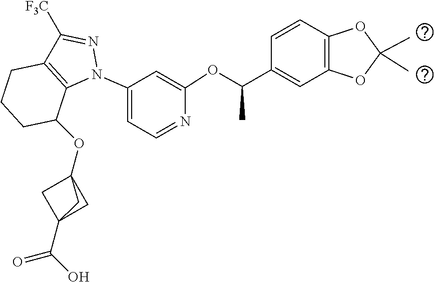 [SMILES: C[C@@H](Oc1cc(-n2nc(C(F)(F)F)c3c2C(OC24CC(C(=O)O)(C2)C4)CCC3)ccn1)c1ccc2c(c1)OC(C)(C)O2] | 3-[[1-[2-[(1R)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 41 | 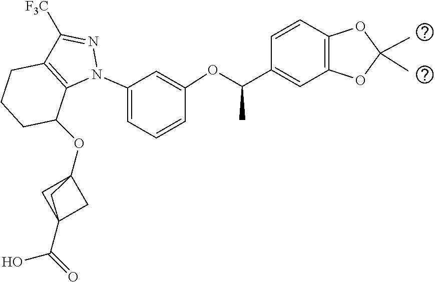 [SMILES: C[C@@H](Oc1cccc(-n2nc(C(F)(F)F)c3c2C(OC24CC(C(=O)O)(C2)C4)CCC3)c1)c1ccc2c(c1)OC(C)(C)O2] | 3-[[1-[3-[(1R)-1-(2,2-difluoro-1,3- benzodioxol-5-yl)ethoxy]phenyl]-3- (trifluoromethyl)-4,5,6,7- tetrahydroindazol-7- yl]oxy]bicyclo[1.1.1]pentane-1- carboxylic acid |
| 42 | 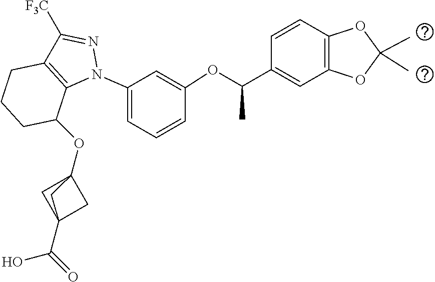 [SMILES: C[C@@H](Oc1cccc(-n2nc(C(F)(F)F)c3c2C(OC24CC(C(=O)O)(C2)C4)CCC3)c1)c1ccc2c(c1)OC(C)(C)O2] | 3-[[1-[3-[(1S)-1-(2,2-difluoro-1,3- benzodioxol-5-yl)ethoxy]phenyl]-3- (trifluoromethyl)-4,5,6,7- tetrahydroindazol-7- yl]oxy]bicyclo[1.1.1]pentane-1- carboxylic acid |
| 43 | 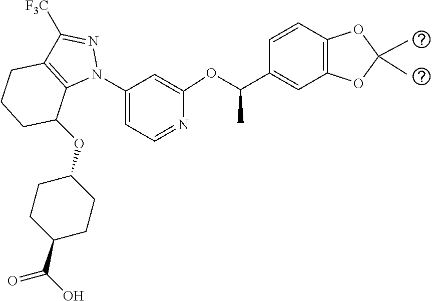 [SMILES: C[C@@H](Oc1cc(-n2nc(C(F)(F)F)c3c2C(O[C@H]2CC[C@H](C(=O)O)CC2)CCC3)ccn1)c1ccc2c(c1)OC(C)(C)O2] | Trans-4-[1-[2-[(1R)-1-(2,2-difluoro-1,3- benzodioxol-5-yl)ethoxy]-4-pyridyl]-3- (trifluoromethyl)-4,5,6,7- tetrahydroindazol-7- yl]oxy]cyclohexanecarboxylic acid |
| 44 | 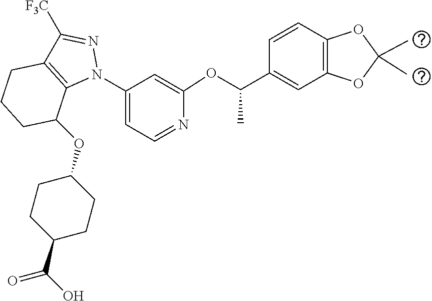 [SMILES: C[C@H](Oc1cc(-n2nc(C(F)(F)F)c3c2C(O[C@H]2CC[C@H](C(=O)O)CC2)CCC3)ccn1)c1ccc2c(c1)OC(C)(C)O2] | Trans-4-[1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]cyclohexanecarboxylic acid |
| 45 | 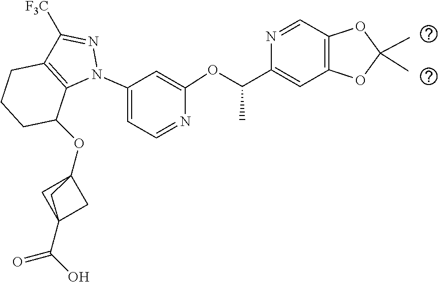 [SMILES: C[C@H](Oc1cc(-n2nc(C(F)(F)F)c3c2C(OC24CC(C(=O)O)(C2)C4)CCC3)ccn1)c1cc2c(cn1)OC(C)(C)O2] | 3-[[1-[2-[(1S)-1-(2,2-difluoro- [1,3]dioxolo[4,5-c]pyridin-6-yl)ethoxy]- 4-pyridyl]-3-(trifluoromethyl)-4,5,6,7- tetrahydroindazol-7- yl]oxy]bicyclo[1.1.1]pentane-1- carboxylic acid |
| 46 | 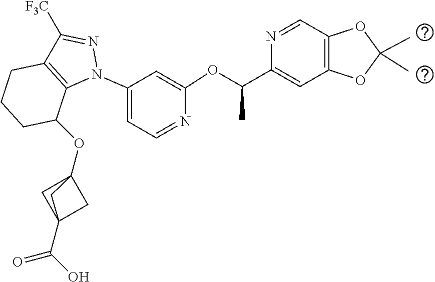 [SMILES: C[C@@H](Oc1cc(-n2nc(C(F)(F)F)c3c2C(OC24CC(C(=O)O)(C2)C4)CCC3)ccn1)c1cc2c(cn1)OC(C)(C)O2] | 3-[[1-[2-[(1R)-1-(2,2-difluoro- [1,3]dioxolo[4,5-c]pyridin-6-yl)ethoxy]- 4-pyridyl]-3-(trifluoromethyl)-4,5,6,7- tetrahydroindazol-7- yl]oxy]bicyclo[1.1.1]pentane-1- carboxylic acid |
| 47 | 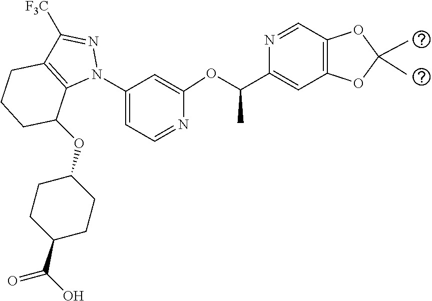 [SMILES: C[C@@H](Oc1cc(-n2nc(C(F)(F)F)c3c2C(O[C@H]2CC[C@H](C(=O)O)CC2)CCC3)ccn1)c1cc2c(cn1)OC(C)(C)O2] | 4-[[1-[2-[(1R)-1-(2,2-difluoro- [1,3]dioxolo[4,5-c]pyridin-6-yl)ethoxy]- 4-pyridyl]-3-(trifluoromethyl)-4,5,6,7- tetrahydroindazol-7- yl]oxy]cyclohexanecarboxylic acid |
| 48 | 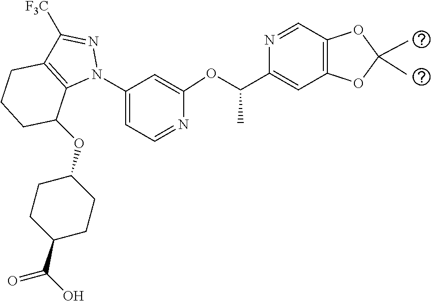 [SMILES: C[C@H](Oc1cc(-n2nc(C(F)(F)F)c3c2C(O[C@H]2CC[C@H](C(=O)O)CC2)CCC3)ccn1)c1cc2c(cn1)OC(C)(C)O2] | Trans-4-[[1-[2-[(1S)-1-(2,2-difluoro- [1,3]dioxolo[4,5-c]pyridin-6-yl)ethoxy]- 4-pyridyl]-3-(trifluoromethyl)-4,5,6,7- tetrahydroindazol-7- yl]oxy]cyclohexanecarboxylic acid |
| 49 | 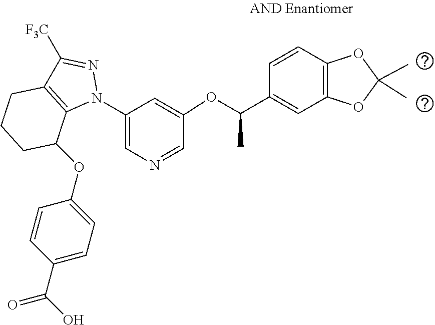 [SMILES: C[C@@H](Oc1cncc(-n2nc(C(F)(F)F)c3c2C(Oc2ccc(C(=O)O)cc2)CCC3)c1)c1ccc2c(c1)OC(C)(C)O2] | 4-[[1-[5-[rac-(1R)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 50 | 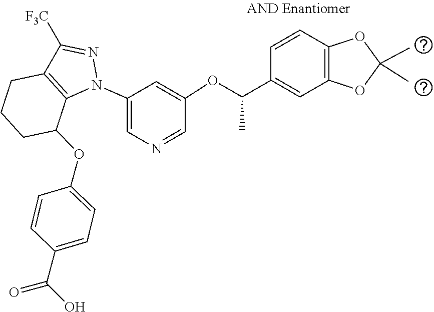 [SMILES: C[C@H](Oc1cncc(-n2nc(C(F)(F)F)c3c2C(Oc2ccc(C(=O)O)cc2)CCC3)c1)c1ccc2c(c1)OC(C)(C)O2] | 4-[[1-[5-[rac-(1S)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 51 | 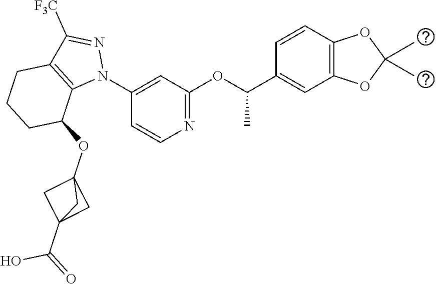 [SMILES: C[C@H](Oc1cc(-n2nc(C(F)(F)F)c3c2[C@@H](OC24CC(C(=O)O)(C2)C4)CCC3)ccn1)c1ccc2c(c1)OC(C)(C)O2] | 3-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 52 | 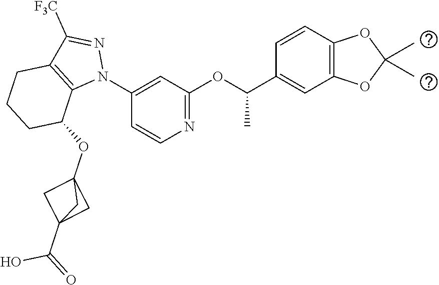 [SMILES: C[C@H](Oc1cc(-n2nc(C(F)(F)F)c3c2[C@H](OC24CC(C(=O)O)(C2)C4)CCC3)ccn1)c1ccc2c(c1)OC(C)(C)O2] | 3-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 53 | 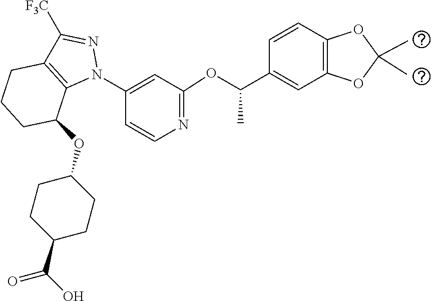 [SMILES: C[C@H](Oc1cc(-n2nc(C(F)(F)F)c3c2[C@@H](O[C@H]2CC[C@H](C(=O)O)CC2)CCC3)ccn1)c1ccc2c(c1)OC(C)(C)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]cyclohexanecarboxylic acid |
| 54 | 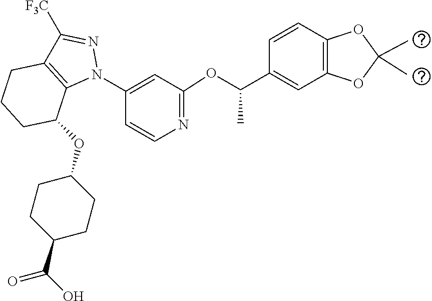 [SMILES: C[C@H](Oc1cc(-n2nc(C(F)(F)F)c3c2[C@H](O[C@H]2CC[C@H](C(=O)O)CC2)CCC3)ccn1)c1ccc2c(c1)OC(C)(C)O2] | Trans-4-[[(7R)-1-[2-[(1S)-1-(2,2- difluoro-1,3-benzodioxol-5-yl)ethoxy]- 4-pyridyl]-3-(trifluoromethyl)-4,5,6,7- tetrahydroindazol-7- yl]oxy]cyclohexanecarboxylic acid |
| 55 | 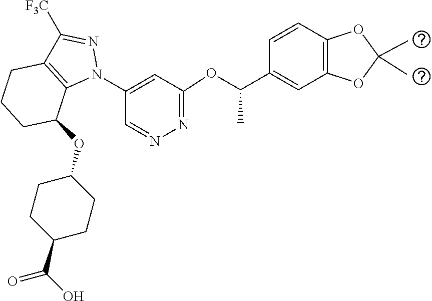 [SMILES: C[C@H](Oc1cc(-n2nc(C(F)(F)F)c3c2[C@@H](O[C@H]2CC[C@H](C(=O)O)CC2)CCC3)cnn1)c1ccc2c(c1)OC(C)(C)O2] | Trans-4-[[(7S)-1-[6-[(1S)-1-(2,2- difluoro-1,3-benzodioxol-5- yl)ethoxy]pyridazin-4-yl]-3- (trifluoromethyl)-4,5,6,7- tetrahydroindazol-7- y]oxy]cyclohexanecarboxylic acid |
| 56 | 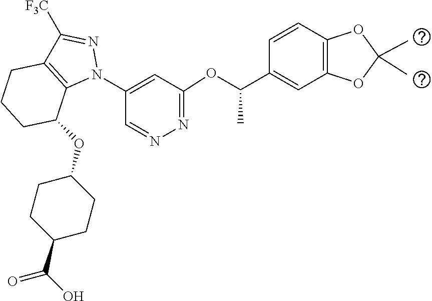 [SMILES: C[C@H](Oc1cc(-n2nc(C(F)(F)F)c3c2[C@H](O[C@H]2CC[C@H](C(=O)O)CC2)CCC3)cnn1)c1ccc2c(c1)OC(C)(C)O2] | Trans-4-[[(7R)-1-[6-[(1S)-1-(2,2- difluoro-1,3-benzodioxol-5- yl)ethoxy]pyridazin-4-yl]-3- (trifluoromethyl)-4,5,6,7- tetrahydroindazol-7- yl]oxy]cyclohexanecarboxylic acid |
| 57 | 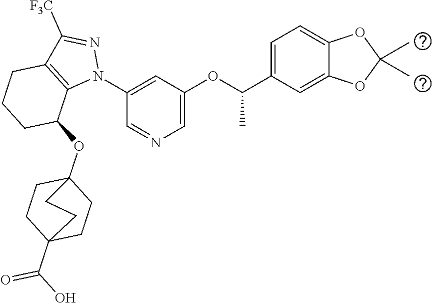 [SMILES: C[C@H](Oc1cncc(-n2nc(C(F)(F)F)c3c2[C@@H](OC24CCC(C(=O)O)(CC2)CC4)CCC3)c1)c1ccc2c(c1)OC(C)(C)O2] | 4-[[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl]oxy]bicyclo[2.2.2]octane-1- carboxylic acid |
| 58 | 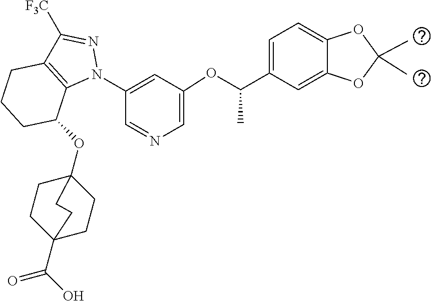 [SMILES: C[C@H](Oc1cncc(-n2nc(C(F)(F)F)c3c2[C@H](OC24CCC(C(=O)O)(CC2)CC4)CCC3)c1)c1ccc2c(c1)OC(C)(C)O2] | 4-[[(7R)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl]oxy]bicyclo[2.2.2]octane-1- carboxylic acid |
| 59 | 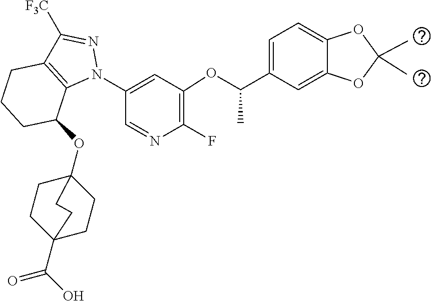 [SMILES: C[C@H](Oc1cc(-n2nc(C(F)(F)F)c3c2[C@@H](OC24CCC(C(=O)O)(CC2)CC4)CCC3)cnc1F)c1ccc2c(c1)OC(C)(C)O2] | 4-[[(7S)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7- yl]oxy]bicyclo[2.2.2]octane-1- carboxylic acid |
| 60 | 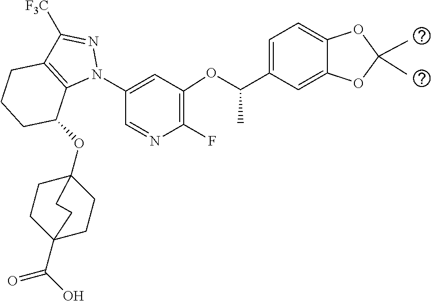 [SMILES: C[C@H](Oc1cc(-n2nc(C(F)(F)F)c3c2[C@H](OC24CCC(C(=O)O)(CC2)CC4)CCC3)cnc1F)c1ccc2c(c1)OC(C)(C)O2] | 4-[[(7R)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7- yl]oxy]bicyclo[2.2.2]octane-1- carboxylic acid |
| 61 | 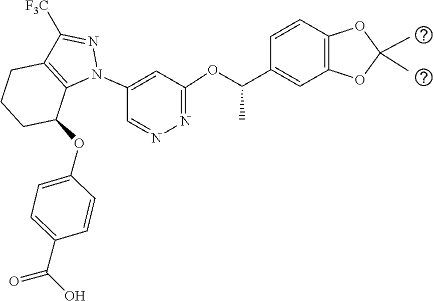 [SMILES: C[C@H](Oc1cc(-n2nc(C(F)(F)F)c3c2[C@@H](Oc2ccc(C(=O)O)cc2)CCC3)cnn1)c1ccc2c(c1)OC(C)(C)O2] | 4-[[(7S)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7-yl]oxy]benzoic acid |
| 62 | 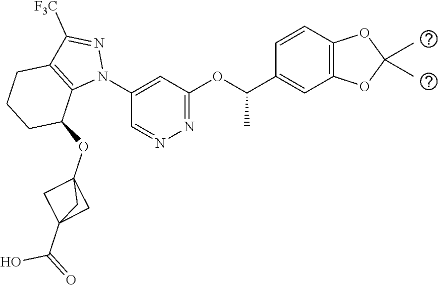 [SMILES: C[C@H](Oc1cc(-n2nc(C(F)(F)F)c3c2[C@@H](OC24CC(C(=O)O)(C2)C4)CCC3)cnn1)c1ccc2c(c1)OC(C)(C)O2] | 3-[[(7S)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7- yl]oxy]bicyclo[1.1.1]pentane-1- carboxylic acid |
| 63 | 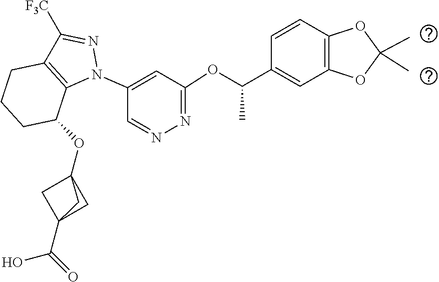 [SMILES: C[C@H](Oc1cc(-n2nc(C(F)(F)F)c3c2[C@H](OC24CC(C(=O)O)(C2)C4)CCC3)cnn1)c1ccc2c(c1)OC(C)(C)O2] | 3-[[(7R)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7- yl]oxy]bicyclo[1.1.1]pentane-1- carboxylic acid |
| 64 | 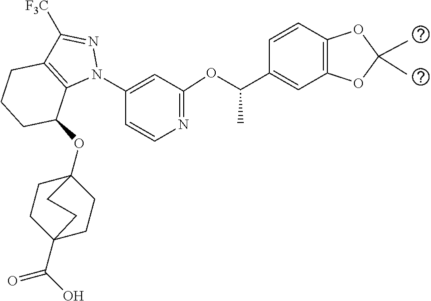 [SMILES: C[C@H](Oc1cc(-n2nc(C(F)(F)F)c3c2[C@@H](OC24CCC(C(=O)O)(CC2)CC4)CCC3)ccn1)c1ccc2c(c1)OC(C)(C)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[2.2.2]octane-1- carboxylic acid |
| 65 | 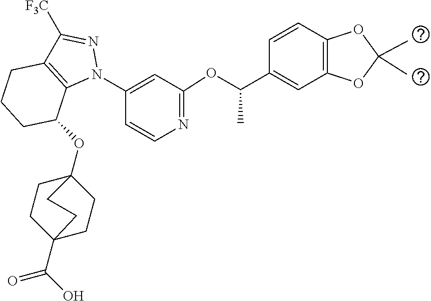 [SMILES: C[C@H](Oc1cc(-n2nc(C(F)(F)F)c3c2[C@H](OC24CCC(C(=O)O)(CC2)CC4)CCC3)ccn1)c1ccc2c(c1)OC(C)(C)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[2.2.2]octane-1- carboxylic acid |
| 66 | 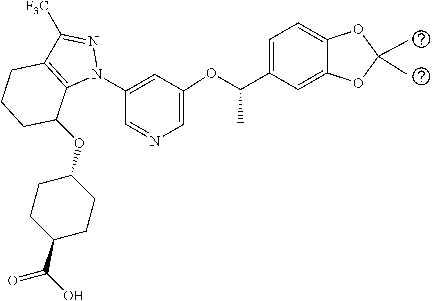 [SMILES: C[C@H](Oc1cncc(-n2nc(C(F)(F)F)c3c2C(O[C@H]2CC[C@H](C(=O)O)CC2)CCC3)c1)c1ccc2c(c1)OC(C)(C)O2] | Trans-4-[[(7S)-1-[5-[(1S)-1-(2,2- difluoro-1,3-benzodioxol-5-yl)ethoxy]- 3-pyridyl]-3-(trifluoromethyl)-4,5,6,7- tetrahydroindazol-7- yl]oxy]cyclohexanecarboxylic acid |
| 67 | 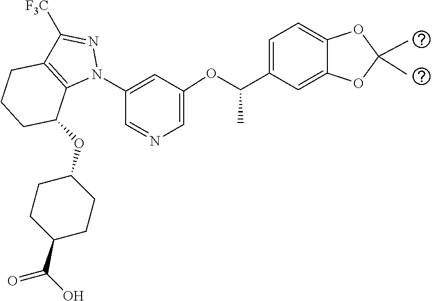 [SMILES: C[C@H](Oc1cncc(-n2nc(C(F)(F)F)c3c2[C@H](O[C@H]2CC[C@H](C(=O)O)CC2)CCC3)c1)c1ccc2c(c1)OC(C)(C)O2] | Trans-4-[[(7R)-1-[5-[(1S)-1-(2,2- difluoro-1,3-benzodioxol-5-yl)ethoxy]- 3-pyridyl]-3-(trifluoromethyl)-4,5,6,7- tetrahydroindazol-7- yl]oxy]cyclohexanecarboxylic acid |
| 68 | 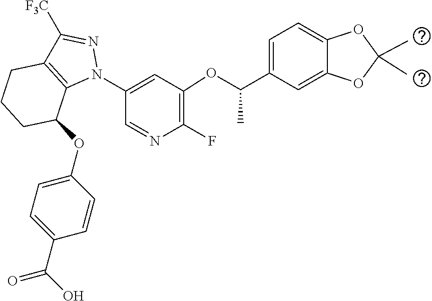 [SMILES: C[C@H](Oc1cc(-n2nc(C(F)(F)F)c3c2[C@@H](Oc2ccc(C(=O)O)cc2)CCC3)cnc1F)c1ccc2c(c1)OC(C)(C)O2] | 4-[[(7S)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7-yl]oxy]benzoic acid |
| 69 | 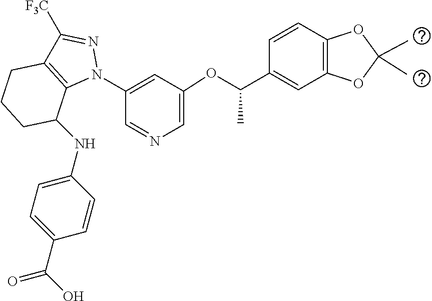 [SMILES: C[C@H](Oc1cncc(-n2nc(C(F)(F)F)c3c2C(Nc2ccc(C(=O)O)cc2)CCC3)c1)c1ccc2c(c1)OC(C)(C)O2] | 4-[[1-[5-[(1S)-1-(2,2-difluoro-1,3- benzodioxol-5-yl)ethoxy]-3-pyridyl]-3- (trifluoromethyl)-4,5,6,7- tetrahydroindazol-7-yl]amino]benzoic acid |
| 70 | 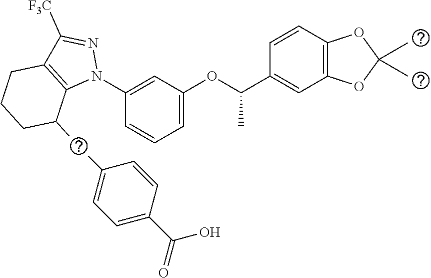 [SMILES: C[C@H](Oc1cccc(-n2nc(C(F)(F)F)c3c2C(Cc2ccc(C(=O)O)cc2)CCC3)c1)c1ccc2c(c1)OC(C)(C)O2] | 4-[[1-[3-[(1S)-1-(2,2-difluoro-1,3- benzodioxol-5-yl)ethoxy]phenyl]-3- (trifluoromethyl)-4,5,6,7- tetrahydroindazol-7-yl]sulfanyl]benzoic acid |
| 71 | 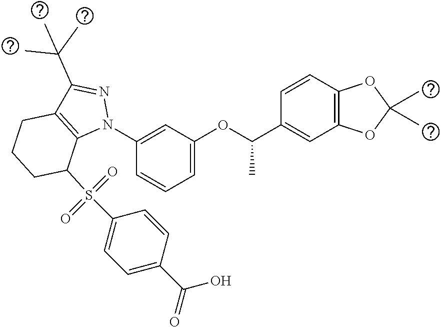 [SMILES: C[C@H](Oc1cccc(-n2nc(C(C)(C)C)c3c2C(S(=O)(=O)c2ccc(C(=O)O)cc2)CCC3)c1)c1ccc2c(c1)OC(C)(C)O2] | 4-[[1-[3-[(1S)-1-(2,2-difluoro-1,3- benzodioxol-5-yl)ethoxy]phenyl]-3- (trifluoromethyl)-4,5,6,7- tetrahydroindazol-7-yl]sulfonyl]benzoic acid |
| 72 | 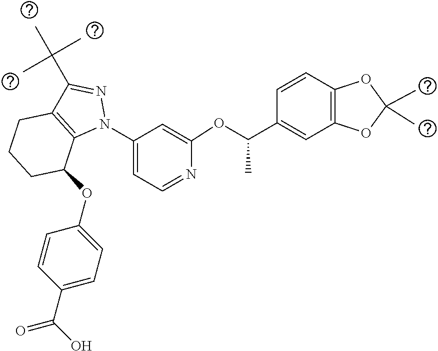 [SMILES: C[C@H](Oc1cc(-n2nc(C(C)(C)C)c3c2[C@@H](Oc2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]sulfanyl]benzoic acid |
| 73 | 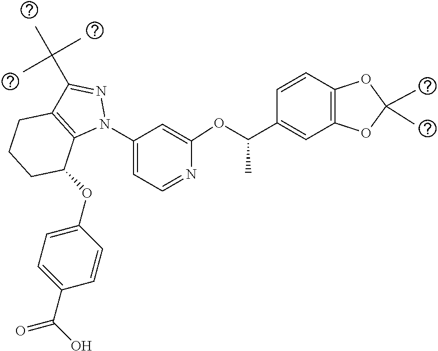 [SMILES: C[C@H](Oc1cc(-n2nc(C(C)(C)C)c3c2[C@H](Oc2ccc(C(=O)O)cc2)CCC3)ccn1)c1ccc2c(c1)OC(C)(C)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]sulfanyl]benzoic acid |
| 74 (P1) | 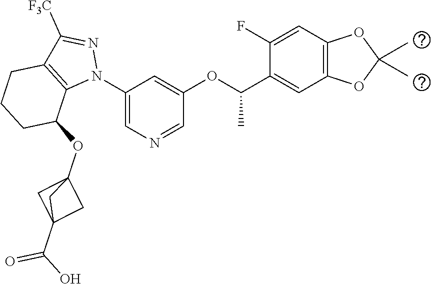 [SMILES: C[C@H](Oc1cncc(-n2nc(C(F)(F)F)c3c2[C@@H](OC24CC(C(=O)O)(C2)C4)CCC3)c1)c1cc2c(cc1F)OC(C)(C)O2] | 3-[[(7S)-1-[5-[(1S)-1-(2,2,6-trifluoro- 1,3-benzodioxol-5-yl)ethoxy]-3- pyridyl]-3-(trifluoromethyl)-4,5,6,7- tetrahydroindazol-7- yl]oxy]bicyclo[1.1.1]pentane-1- carboxylic acid |
| 74 (P2) | 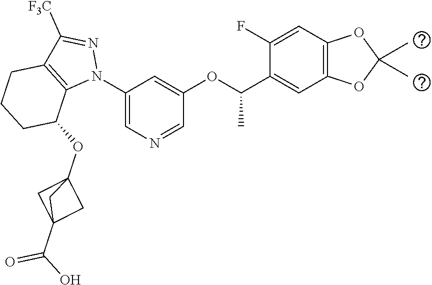 [SMILES: C[C@H](Oc1cncc(-n2nc(C(F)(F)F)c3c2[C@H](OC24CC(C(=O)O)(C2)C4)CCC3)c1)c1cc2c(cc1F)OC(C)(C)O2] | 3-[[(7R)-1-[5-[(1S)-1-(2,2,6-trifluoro- 1,3-benzodioxol-5-yl)ethoxy]-3- pyridyl]-3-(trifluoromethyl)-4,5,6,7- tetrahydroindazol-7- yl]oxy]bicyclo[1.1.1]pentane-1- carboxylic acid |
| 75 (P1) | 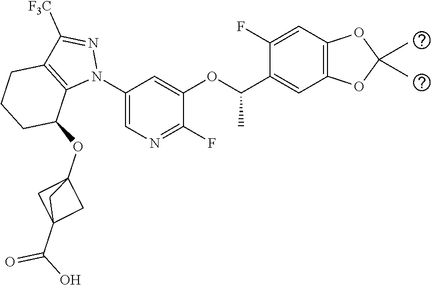 [SMILES: C[C@H](Oc1cc(-n2nc(C(F)(F)F)c3c2[C@@H](OC24CC(C(=O)O)(C2)C4)CCC3)cnc1F)c1cc2c(cc1F)OC(C)(C)O2] | 3-[[(7S)-1-[6-fluoro-5-[(1S)-1-(2,2,6- trifluoro-1,3-benzodioxol-5-yl)ethoxy]- 3-pyridyl]-3-(trifluoromethyl)-4,5,6,7- tetrahydroindazol-7- yl]oxy]bicyclo[1.1.1]pentane-1- carboxylic acid |
| 75 (P2) | 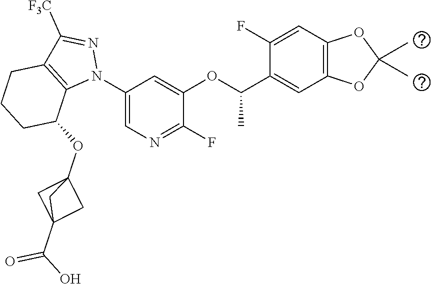 [SMILES: C[C@H](Oc1cc(-n2nc(C(F)(F)F)c3c2[C@H](OC24CC(C(=O)O)(C2)C4)CCC3)cnc1F)c1cc2c(cc1F)OC(C)(C)O2] | 3-[[(7R)-1-[6-fluoro-5-[(1S)-1-(2,2,6- trifluoro-1,3-benzodioxol-5-yl)ethoxy]- 3-pyridyl]-3-(trifluoromethyl)-4,5,6,7- tetrahydroindazol-7- yl]oxy]bicyclo[1.1.1]pentane-1- carboxylic acid |
| 76 (P1) | 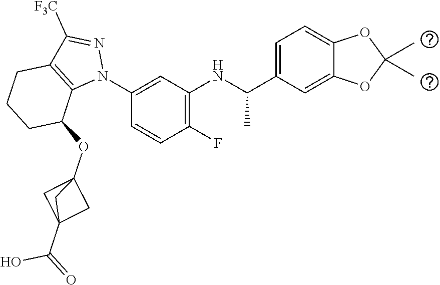 [SMILES: C[C@H](Nc1cc(-n2nc(C(F)(F)F)c3c2[C@@H](OC24CC(C(=O)O)(C2)C4)CCC3)ccc1F)c1ccc2c(c1)OC(C)(C)O2] | 3-[[(7S)-1-[3-[(1S)-1-(2,2-difluoro-1,3- benzodioxol-5-yl)ethoxy]-4-fluoro- phenyl]-3-(trifluoromethyl)-4,5,6,7- tetrahydroindazol-7- yl]oxy]bicyclo[1.1.1]pentane-1- carboxylic acid |
| 76 (P2) | 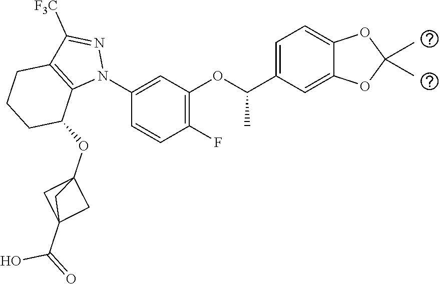 [SMILES: C[C@H](Oc1cc(-n2nc(C(F)(F)F)c3c2[C@H](OC24CC(C(=O)O)(C2)C4)CCC3)ccc1F)c1ccc2c(c1)OC(C)(C)O2] | 3-[[(7R)-1-[3-[(1S)-1-(2,2-difluoro-1,3- benzodioxol-5-yl)ethoxy]-4-fluoro- phenyl]-3-(trifluoromethyl)-4,5,6,7- tetrahydroindazol-7- yloxy]bicyclo[1.1.1]pentane-1- carboxylic acid |
| 77 (P1) | 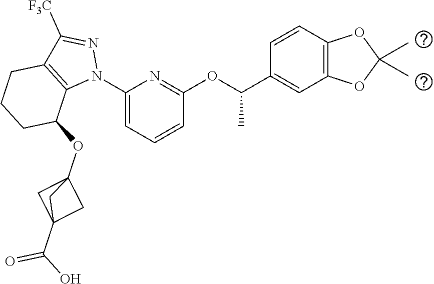 [SMILES: C[C@H](Oc1cccc(-n2nc(C(F)(F)F)c3c2[C@@H](OC24CC(C(=O)O)(C2)C4)CCC3)n1)c1ccc2c(c1)OC(C)(C)O2] | 3-[[(7S)-1-[6-[(1S)-1-(2,2-difluoro-1,3- benzodioxol-5-yl)ethoxy]-2-pyridyl]-3- (trifluoromethyl)-4,5,6,7- tetrahydroindazol-7- yl]oxy]bicyclo[1.1.1]pentane-1- carboxylic acid |
| 77 (P2) | 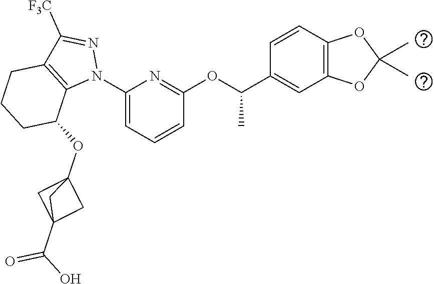 [SMILES: C[C@H](Oc1cccc(-n2nc(C(F)(F)F)c3c2[C@H](OC24CC(C(=O)O)(C2)C4)CCC3)n1)c1ccc2c(c1)OC(C)(C)O2] | 3-[[(7R)-1-[6-[(1S)-1-(2,2-difluoro-1,3- benzodioxol-5-yl)ethoxy]-2-pyridyl]-3- (trifluoromethyl)-4,5,6,7- tetrahydroindazol-7- yl]oxy]bicyclo[1.1.1]pentane-1- carboxylic acid |
| 78 (P1) | 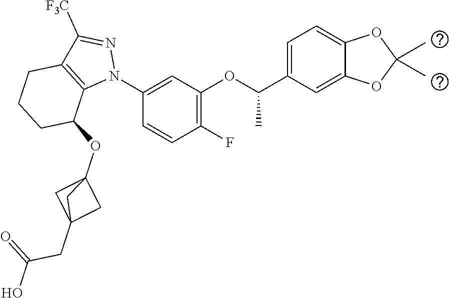 [SMILES: C[C@H](Oc1cc(-n2nc(C(F)(F)F)c3c2[C@@H](OC24CC(CC(=O)O)(C2)C4)CCC3)ccc1F)c1ccc2c(c1)OC(C)(C)O2] | 2-[3-[[(7S)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]-1- bicyclo[1.1.1]pentanyl]acetic acid |
| 78 (P2) | 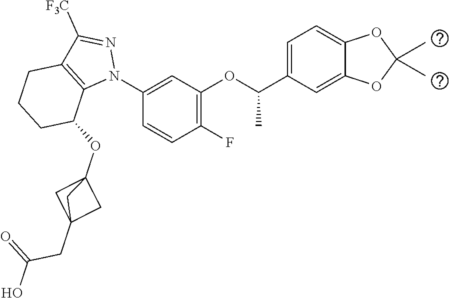 [SMILES: C[C@H](Oc1cc(-n2nc(C(F)(F)F)c3c2[C@H](OC24CC(CC(=O)O)(C2)C4)CCC3)ccc1F)c1ccc2c(c1)OC(C)(C)O2] | 2-[3-[[(7R)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]-1- bicyclo[1.1.1]pentanyl]acetic acid |
| 79 (P1) | 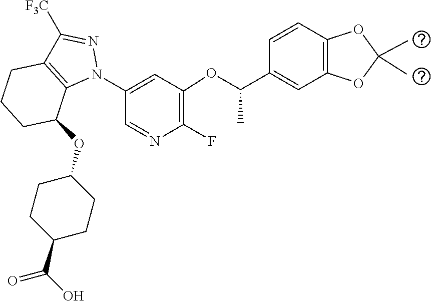 [SMILES: C[C@H](Oc1cc(-n2nc(C(F)(F)F)c3c2[C@@H](O[C@H]2CC[C@H](C(=O)O)CC2)CCC3)cnc1F)c1ccc2c(c1)OC(C)(C)O2] | Trans-4-[[(7S)-1-[5-[(1S)-1-(2,2- difluoro-1,3-benzodioxol-5-yl)ethoxy]- 6-fluoro-3-pyridyl]-3-(trifluoromethyl)- 4,5,6,7-tetrahydroindazol-7- yl]oxy]cyclohexanecarboxylic acid |
| 79 (P2) | 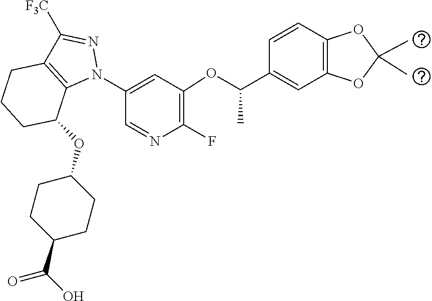 [SMILES: C[C@H](Oc1cc(-n2nc(C(F)(F)F)c3c2[C@H](O[C@H]2CC[C@H](C(=O)O)CC2)CCC3)cnc1F)c1ccc2c(c1)OC(C)(C)O2] | Trans-4-[(7R)-1-[5-[(1S)-1-(2,2- difluoro-1,3-benzodioxol-5-yl)ethoxy]- 6-fluoro-3-pyridyl]-3-(trifluoromethyl)- 4,5,6,7-tetrahydroindazol-7- yl]oxy]cyclohexanecarboxylic acid |
| 80 (P1) | 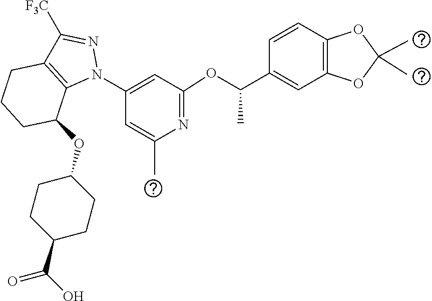 [SMILES: Cc1cc(-n2nc(C(F)(F)F)c3c2[C@@H](O[C@H]2CC[C@H](C(=O)O)CC2)CCC3)cc(O[C@@H](C)c2ccc3c(c2)OC(C)(C)O3)n1] | Trans-4-[[(7S)-1-[2-[(1S)-1-(2,2- difluoro-1,3-benzodioxol-5-yl)ethoxy]- 6-fluoro-4-pyridyl]-3-(trifluoromethyl)- 4,5,6,7-tetrahydroindazol-7- yl]oxy]cyclohexanecarboxylic acid |
| 80 (P2) | 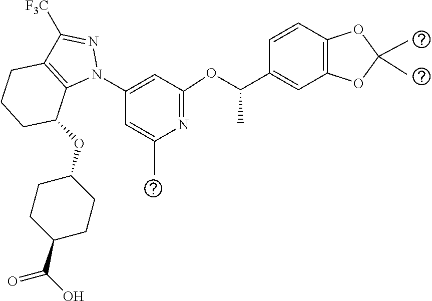 [SMILES: Cc1cc(-n2nc(C(F)(F)F)c3c2[C@H](O[C@H]2CC[C@H](C(=O)O)CC2)CCC3)cc(O[C@@H](C)c2ccc3c(c2)OC(C)(C)O3)n1] | Trans-4-[[(7R)-1-[2-[(1S)-1-(2,2- difluoro-1,3-benzodioxol-5-yl)ethoxy]- 6-fluoro-4-pyridyl]-3-(trifluoromethyl)- 4,5,6,7-tetrahydroindazol-7- yl]oxy]cyclohexanecarboxylic acid |
| 81 (P1) | 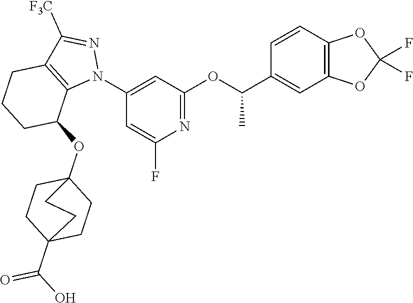 [SMILES: C[C@H](Oc1cc(-n2nc(C(F)(F)F)c3c2[C@@H](OC24CCC(C(=O)O)(CC2)CC4)CCC3)cc(F)n1)c1ccc2c(c1)OC(F)(F)O2] | 4-[[(7S)-1-[2-[(1S)-1-(2,2-difluoro-1,3- benzodioxol-5-yl)ethoxy]-6-fluoro-4- pyridyl]-3-(trifluoromethyl)-4,5,6,7- tetrahydroindazol-7- yl]oxy]bicyclo[2.2.2]octane-1- carboxylic acid |
| 81 (P2) | 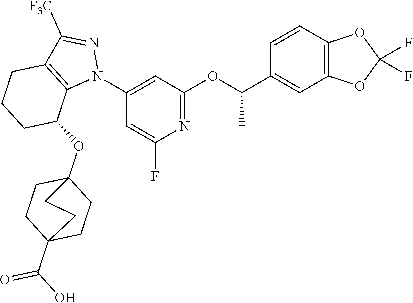 [SMILES: C[C@H](Oc1cc(-n2nc(C(F)(F)F)c3c2[C@H](OC24CCC(C(=O)O)(CC2)CC4)CCC3)cc(F)n1)c1ccc2c(c1)OC(F)(F)O2] | 4-[[(7R)-1-[2-[(1S)-1-(2,2-difluoro-1,3- benzodioxol-5-yl)ethoxy]-6-fluoro-4- pyridyl]-3-(trifluoromethyl)-4,5,6,7- tetrahydroindazol-7- yl]oxy]bicyclo[2.2.2]octane-1- carboxylic acid |
| 82 (P1) | 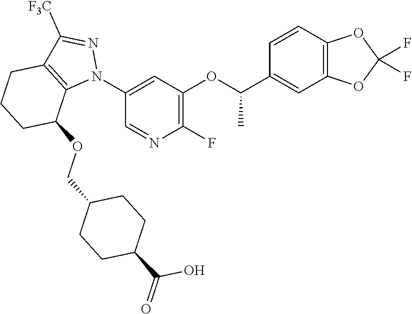 [SMILES: C[C@H](Oc1cc(-n2nc(C(F)(F)F)c3c2[C@@H](OC[C@H]2CC[C@H](C(=O)O)CC2)CCC3)cnc1F)c1ccc2c(c1)OC(F)(F)O2] | Trans-4-[[(7S)-1-[5-[(1S)-1-(2,2- difluoro-1,3-benzodioxol-5-yl)ethoxy]- 6-fluoro-3-pyridyl]-3-(trifluoromethyl)- 4,5,6,7-tetrahydroindazol-7- yl]oxymethyl]cyclohexanecarboxylic acid |
| 82 (P2) | 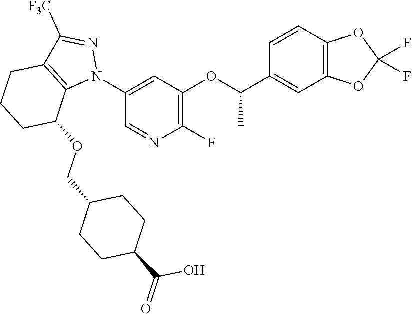 [SMILES: C[C@H](Oc1cc(-n2nc(C(F)(F)F)c3c2[C@H](OC[C@H]2CC[C@H](C(=O)O)CC2)CCC3)cnc1F)c1ccc2c(c1)OC(F)(F)O2] | Trans-4-[[(7R)-1-[5-[(1S)-1-(2,2- difluoro-1,3-benzodioxol-5-yl)ethoxy]- 6-fluoro-3-pyridyl]-3-(trifluoromethyl)- 4,5,6,7-tetrahydroindazol-7- yl]oxymethyl]cyclohexanecarboxylic acid |
| 83 (P1) | 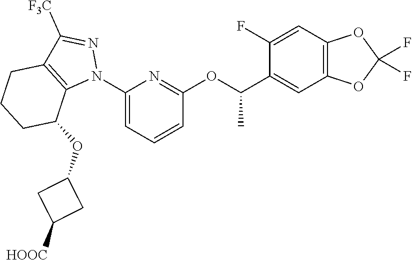 [SMILES: C[C@H](Oc1cccc(-n2nc(C(F)(F)F)c3c2[C@H](O[C@H]2C[C@H](C(=O)O)C2)CCC3)n1)c1cc2c(cc1F)OC(F)(F)O2] | Trans-3-[[(7S)-1-[6-[(1S)-1-(2,2,6- trifluoro-1,3-benzodioxol-5-yl)ethoxy]- 2-pyridyl]-3-(trifluoromethyl)-4,5,6,7- tetrahydroindazol-7- yl]oxy]cyclobutanecarboxylic acid |
| 83 (P2) | 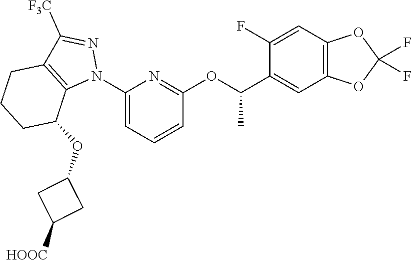 [SMILES: C[C@H](Oc1cccc(-n2nc(C(F)(F)F)c3c2[C@H](O[C@H]2C[C@H](C(=O)O)C2)CCC3)n1)c1cc2c(cc1F)OC(F)(F)O2] | Trans-3-[[(7R)-1-[6-[(1S)-1-(2,2,6- trifluoro-1,3-benzodioxol-5-yl)ethoxy]- 2-pyridyl]-3-(trifluoromethyl)-4,5,6,7- tetrahydroindazol-7- yl]oxy]cyclobutanecarboxylic acid |
| 84 (P1) | 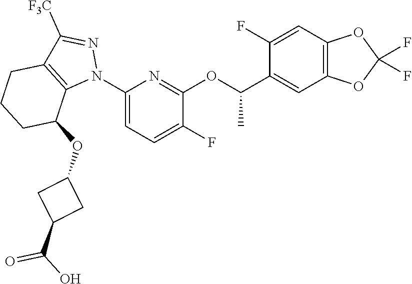 [SMILES: C[C@H](Oc1nc(-n2nc(C(F)(F)F)c3c2[C@@H](O[C@H]2C[C@H](C(=O)O)C2)CCC3)ccc1F)c1cc2c(cc1F)OC(F)(F)O2] | Trans-3-[[(7S)-1-[5-fluoro-6-[(1S)-1- (2,2,6-trifluoro-1,3-benzodioxol-5- yl)ethoxy]-2-pyridyl]-3- (trifluoromethyl)-4,5,6,7- tetrahydroindazol-7- yl]oxy]cyclobutanecarboxylic acid |
| 84 (P2) | 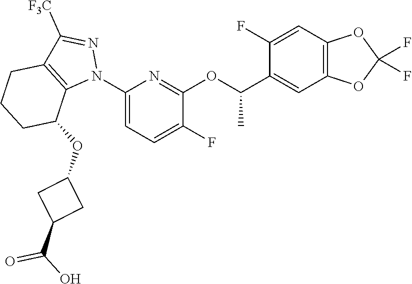 [SMILES: C[C@H](Oc1nc(-n2nc(C(F)(F)F)c3c2[C@H](O[C@H]2C[C@H](C(=O)O)C2)CCC3)ccc1F)c1cc2c(cc1F)OC(F)(F)O2] | Trans-3-[(7R)-1-[5-fluoro-6-[(1S)-1- (2,2,6-trifluoro-1,3-benzodioxol-5- yl)ethoxy]-2-pyridyl]-3- (trifluoromethyl)-4,5,6,7- tetrahydroindazol-7- yl]oxy]cyclobutanecarboxylic acid |
| 85 (P1) | 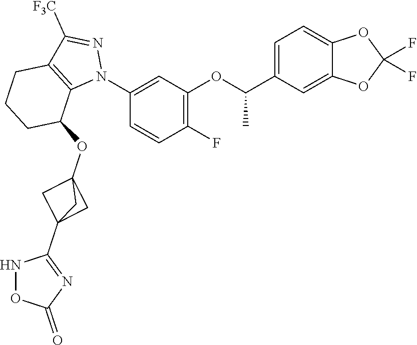 [SMILES: C[C@H](Oc1cc(-n2nc(C(F)(F)F)c3c2[C@@H](OC24CC(c5nc(=O)o[nH]5)(C2)C4)CCC3)ccc1F)c1ccc2c(c1)OC(F)(F)O2] | 3-[3-[[(7S)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]-1- bicyclo[1.1.1]pentanyl]-2H-1,2,4- oxadiazol-5-one |
| 85 (P2) | 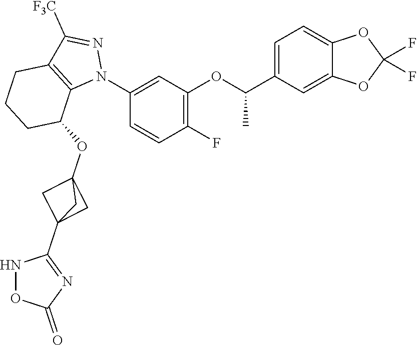 [SMILES: C[C@H](Oc1cc(-n2nc(C(F)(F)F)c3c2[C@H](OC24CC(c5nc(=O)o[nH]5)(C2)C4)CCC3)ccc1F)c1ccc2c(c1)OC(F)(F)O2] | 3-[3-[[(7R)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]-1- bicyclo[1.1.1]pentanyl]-2H-1,2,4- oxadiazol-5-one |
| 86 (P1) | 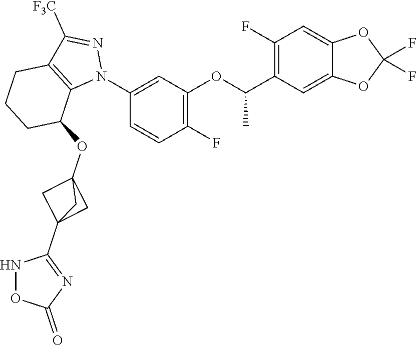 [SMILES: C[C@H](Oc1cc(-n2nc(C(F)(F)F)c3c2[C@@H](OC24CC(c5nc(=O)o[nH]5)(C2)C4)CCC3)ccc1F)c1cc2c(cc1F)OC(F)(F)O2] | 3-[3-[[(7S)-1-[4-fluoro-3-[(1S)-1-(2,2,6- trifluoro-1,3-benzodioxol-5- yl)ethoxy]phenyl]-3-(trifluoromethyl)- 4,5,6,7-tetrahydroindazol-7-yl]oxy]-1- bicyclo[1.1.1]pentanyl]-2H-1,2,4- oxadiazol-5-one |
| 86 (P2) | 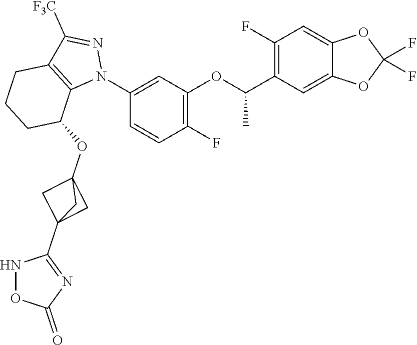 [SMILES: C[C@H](Oc1cc(-n2nc(C(F)(F)F)c3c2[C@H](OC24CC(c5nc(=O)o[nH]5)(C2)C4)CCC3)ccc1F)c1cc2c(cc1F)OC(F)(F)O2] | 3-[3-[[(7R)-1-[4-fluoro-3-[(1S)-1-(2,2,6- trifluoro-1,3-benzodioxol-5- yl)ethoxyphenyl]-3-(trifluoromethyl)- 4,5,6,7-tetrahydroindazol-7-yl]oxy]-1- bicyclo[1.1.1]pentanyl]-2H-1,2,4- oxadiazol-5-one |
| 87 | 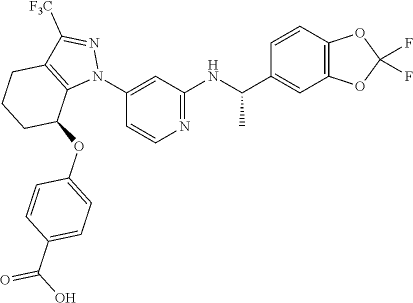 [SMILES: C[C@H](Nc1cc(-n2nc(C(F)(F)F)c3c2[C@@H](Oc2ccc(C(=O)O)cc2)CCC3)ccn1)c1ccc2c(c1)OC(F)(F)O2] | 4-[[(7S)-1-[2-[[(1S)-1-(2,2-difluoro-1,3- benzodioxol-5-yl)ethyl]amino]-4- pyridyl]-3-(trifluoromethyl)-4,5,6,7- tetrahydroindazol-7-yl]oxy]benzoic acid |
| 88 | 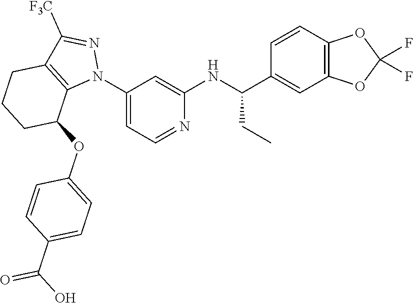 [SMILES: CC[C@H](Nc1cc(-n2nc(C(F)(F)F)c3c2[C@@H](Oc2ccc(C(=O)O)cc2)CCC3)ccn1)c1ccc2c(c1)OC(F)(F)O2] | 4-[[(7S)-1-[2-[[(1S)-1-(2,2-difluoro-1,3- benzodioxol-5-yl)propyl]amino]-4- pyridyl]-3-(trifluoromethyl)-4,5,6,7- tetrahydroindazol-7-yl]oxy]benzoic acid |
| 89 | 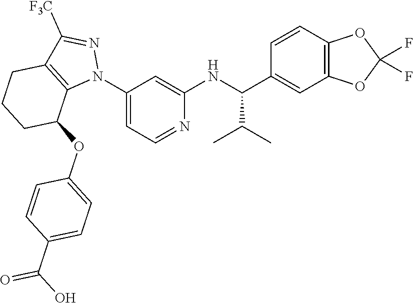 [SMILES: CC(C)[C@H](Nc1cc(-n2nc(C(F)(F)F)c3c2[C@@H](Oc2ccc(C(=O)O)cc2)CCC3)ccn1)c1ccc2c(c1)OC(F)(F)O2] | 4-[[(7S)-1-[2-[(1S)-1-(2,2-difluoro-1,3- benzodioxol-5-yl)-2-methyl- propyl]amino]-4-pyridyl]-3- (trifluoromethyl)-4,5,6,7- tetrahydroindazol-7-yl]oxy]benzoic acid |
| 90 | 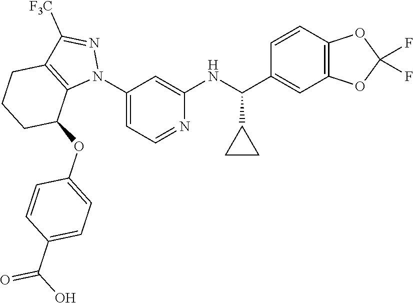 [SMILES: O=C(O)c1ccc(O[C@H]2CCCc3c(C(F)(F)F)nn(-c4ccnc(N[C@H](c5ccc6c(c5)OC(F)(F)O6)C5CC5)c4)c32)cc1] | 4-[[(7S)-1-[2-[[(S)-cyclopropyl-(2,2- difluoro-1,3-benzodioxol-5- yl)methyl]amino]-4-pyridyl]-3- (trifluoromethyl)-4,5,6,7- tetrahydroindazol-7-yl]oxy]benzoic acid |
| 91 | 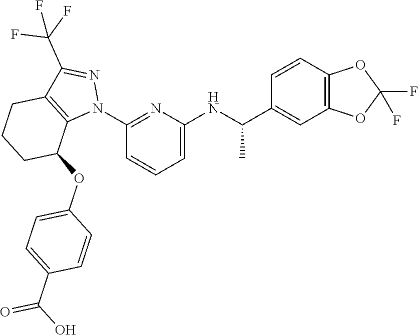 [SMILES: C[C@H](Nc1cccc(-n2nc(C(F)(F)F)c3c2[C@@H](Oc2ccc(C(=O)O)cc2)CCC3)n1)c1ccc2c(c1)OC(F)(F)O2] | 4-[[(7S)-1-[6-[[(1S)-1-(2,2-difluoro-1,3- benzodioxol-5-yl)ethyl]amino]-2- pyridyl]-3-(trifluoromethyl)-4,5,6,7- tetrahydroindazol-7-yl]oxy]benzoic acid |
| 92 | 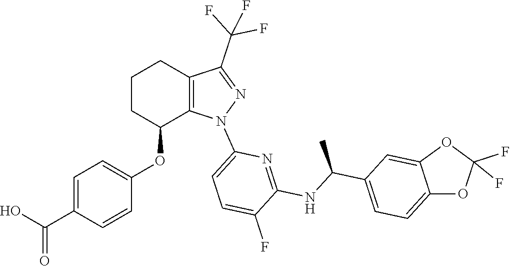 [SMILES: C[C@H](Nc1nc(-n2nc(C(F)(F)F)c3c2[C@@H](Oc2ccc(C(=O)O)cc2)CCC3)ccc1F)c1ccc2c(c1)OC(F)(F)O2] | 4-[[(7S)-1-[6-[[(1S)-1-(2,2-difluoro-1,3- benzodioxol-5-yl)ethyl]amino]-5-fluoro- 2-pyridyl]-3-(trifluoromethyl)-4,5,6,7- tetrahydroindazol-7-yl]oxy]benzoic acid |
| 93 | 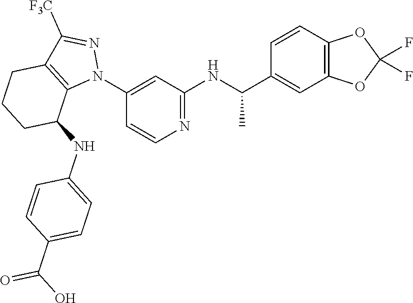 [SMILES: C[C@H](Nc1cc(-n2nc(C(F)(F)F)c3c2[C@@H](Nc2ccc(C(=O)O)cc2)CCC3)ccn1)c1ccc2c(c1)OC(F)(F)O2] | 4-[[(7S)-1-[2-[[(1S)-1-(2,2-difluoro-1,3- benzodioxol-5-yl)ethyl]amino]-4- pyridyl]-3-(trifluoromethyl)-4,5,6,7- tetrahydroindazol-7-yl]amino]benzoic acid |
| 94 (P1) | 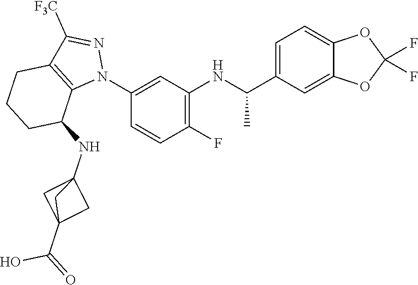 [SMILES: C[C@H](Nc1cc(-n2nc(C(F)(F)F)c3c2[C@@H](NC24CC(C(=O)O)(C2)C4)CCC3)ccc1F)c1ccc2c(c1)OC(F)(F)O2] | 3-[(7S)-1-[3-[[(1S)-1-(2,2-difluoro-1,3- benzodioxol-5-yl)ethyl]amino]-4-fluoro- phenyl]-3-(trifluoromethyl)-4,5,6,7- tetrahydroindazol-7- yl]amino]bicyclo[1.1.1]pentane-1- carboxylic acid |
| 94 (P2) | 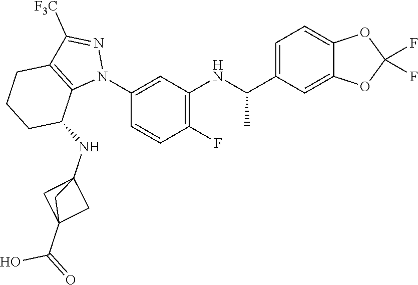 [SMILES: C[C@H](Nc1cc(-n2nc(C(F)(F)F)c3c2[C@H](NC24CC(C(=O)O)(C2)C4)CCC3)ccc1F)c1ccc2c(c1)OC(F)(F)O2] | 3-[[(7R)-1-[3-[[(1S)-1-(2,2-difluoro-1,3- benzodioxol-5-yl)ethyl]amino]-4-fluoro- phenyl]-3-(trifluoromethyl)-4,5,6,7- tetrahydroindazol-7- yl]amino]bicyclo[1.1.1]pentane-1- carboxylic acid |
| 95 (P1) | 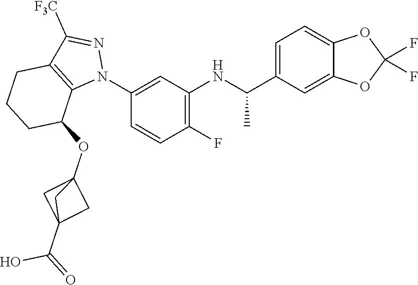 [SMILES: C[C@H](Nc1cc(-n2nc(C(F)(F)F)c3c2[C@@H](OC24CC(C(=O)O)(C2)C4)CCC3)ccc1F)c1ccc2c(c1)OC(F)(F)O2] | 3-[[(7S)-1-[3-[(1S)-1-(2,2-difluoro-1,3- benzodioxol-5-yl)ethyl]amino]-4-fluoro- phenyl]-3-(trifluoromethyl)-4,5,6,7- tetrahydroindazol-7- yl]oxy]bicyclo[1.1.1]pentane-1- carboxylic acid |
| 95 (P2) | 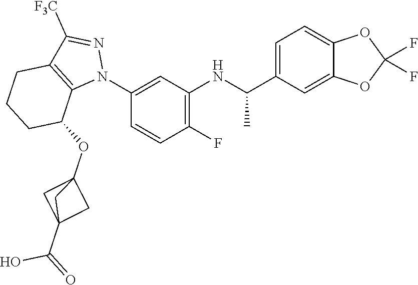 [SMILES: C[C@H](Nc1cc(-n2nc(C(F)(F)F)c3c2[C@H](OC24CC(C(=O)O)(C2)C4)CCC3)ccc1F)c1ccc2c(c1)OC(F)(F)O2] | 3-[[(7R)-1-[3-[(1S)-1-(2,2-difluoro-1,3- benzodioxol-5-yl)ethyl]amino]-4-fluoro- phenyl]-3-(trifluoromethyl)-4,5,6,7- tetrahydroindazol-7- yl]oxy]bicyclo[1.1.1]pentane-1- carboxylic acid |
| 96 (P1) | 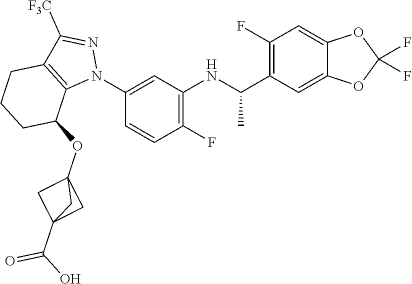 [SMILES: C[C@H](Nc1cc(-n2nc(C(F)(F)F)c3c2[C@@H](OC24CC(C(=O)O)(C2)C4)CCC3)ccc1F)c1cc2c(cc1F)OC(F)(F)O2] | 3-[[(7S)-1-[4-fluoro-3-[[(1S)-1-(2,2,6- trifluoro-1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 96 (P2) | 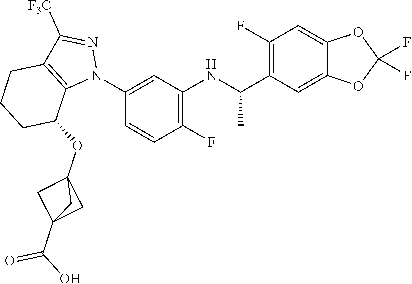 [SMILES: C[C@H](Nc1cc(-n2nc(C(F)(F)F)c3c2[C@H](OC24CC(C(=O)O)(C2)C4)CCC3)ccc1F)c1cc2c(cc1F)OC(F)(F)O2] | 3-[[(7R)-1-[4-fluoro-3-[[(1S)-1-(2,2,6- trifluoro-1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 97 (P1) | 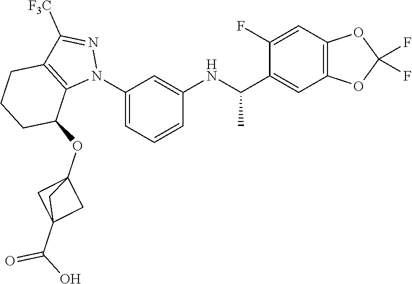 [SMILES: C[C@H](Nc1cccc(-n2nc(C(F)(F)F)c3c2[C@@H](OC24CC(C(=O)O)(C2)C4)CCC3)c1)c1cc2c(cc1F)OC(F)(F)O2] | 3-[[(7S)-1-[3-[(1S)-1-(2,2,6-trifluoro- 1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 97 (P2) | 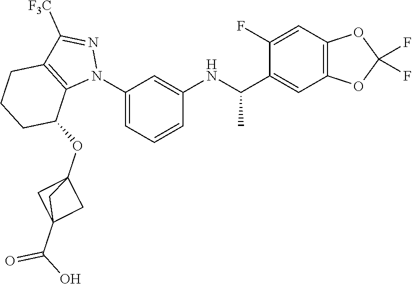 [SMILES: C[C@H](Nc1cccc(-n2nc(C(F)(F)F)c3c2[C@H](OC24CC(C(=O)O)(C2)C4)CCC3)c1)c1cc2c(cc1F)OC(F)(F)O2] | 3-[[(7R)-1-[3-[(1S)-1-(2,2,6-trifluoro- 1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 98 (P1) | 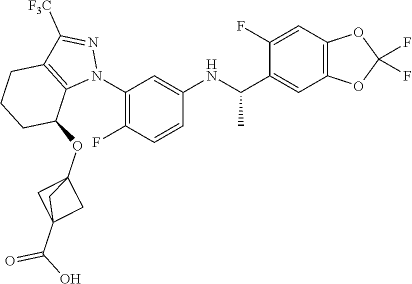 [SMILES: C[C@H](Nc1ccc(F)c(-n2nc(C(F)(F)F)c3c2[C@@H](OC24CC(C(=O)O)(C2)C4)CCC3)c1)c1cc2c(cc1F)OC(F)(F)O2] | 3-[[(7S)-1-[2-fluoro-5-[[(1S)-1-(2,2,6- trifluoro-1,3-benzodioxol-5- yl)ethyl]amino]phenyl]-3- (trifluoromethyl)-4,5,6,7- tetrahydroindazol-7- yloxy]bicyclo[1.1.1]pentane-1- carboxylic acid |
| 98 (P2) | 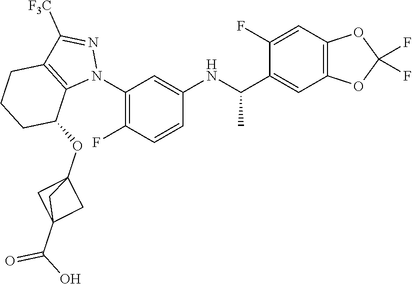 [SMILES: C[C@H](Nc1ccc(F)c(-n2nc(C(F)(F)F)c3c2[C@H](OC24CC(C(=O)O)(C2)C4)CCC3)c1)c1cc2c(cc1F)OC(F)(F)O2] | 3-[[(7R)-1-[2-fluoro-5-[[(1S)-1-(2,2,6- trifluoro-1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 99 (P1) | 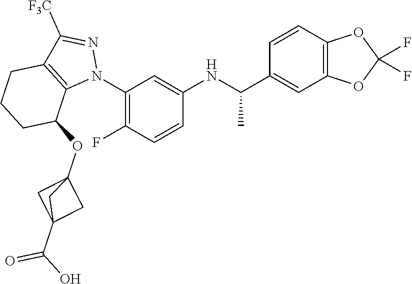 [SMILES: C[C@H](Nc1ccc(F)c(-n2nc(C(F)(F)F)c3c2[C@@H](OC24CC(C(=O)O)(C2)C4)CCC3)c1)c1ccc2c(c1)OC(F)(F)O2] | 3-[[(7S)-1-[5-[[(1S)-1-(2,2-difluoro-1,3- benzodioxol-5-yl)ethyl]amino]-2-fluoro- phenyl]-3-(trifluoromethyl)-4,5,6,7- tetrahydroindazol-7- yl]oxy]bicyclo[1.1.1]pentane-1- carboxylic acid |
| 99 (P2) | 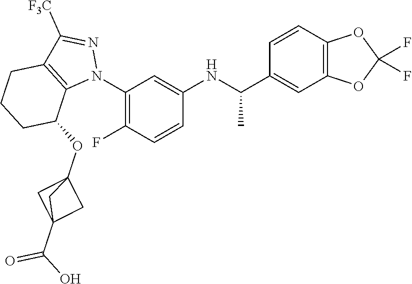 [SMILES: C[C@H](Nc1ccc(F)c(-n2nc(C(F)(F)F)c3c2[C@H](OC24CC(C(=O)O)(C2)C4)CCC3)c1)c1ccc2c(c1)OC(F)(F)O2] | 3-[(7R)-1-[5-[(1S)-1-(2,2-difluoro-1,3- benzodioxol-5-yl)ethyl]amino]-2-fluoro- phenyl]-3-(trifluoromethyl)-4,5,6,7- tetrahydroindazol-7- yl]oxy]bicyclo[1.1.1]pentane-1- carboxylic acid |
| 100 (P1) | 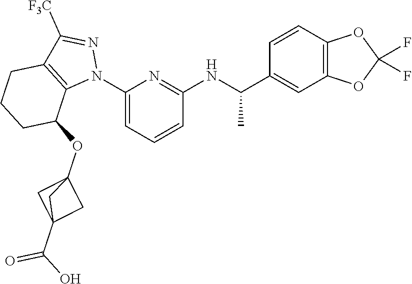 [SMILES: C[C@H](Nc1cccc(-n2nc(C(F)(F)F)c3c2[C@@H](OC24CC(C(=O)O)(C2)C4)CCC3)n1)c1ccc2c(c1)OC(F)(F)O2] | 3-[[(7S)-1-[6-[[(1S)-1-(2,2-difluoro-1,3- benzodioxol-5-yl)ethyl]amino]-2- pyridyl]-3-(trifluoromethyl)-4,5,6,7- tetrahydroindazol-7- yl]oxy]bicyclo[1.1.1]pentane-1- carboxylic acid |
| 100 (P2) | 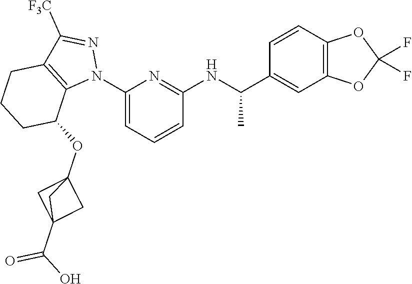 [SMILES: C[C@H](Nc1cccc(-n2nc(C(F)(F)F)c3c2[C@H](OC24CC(C(=O)O)(C2)C4)CCC3)n1)c1ccc2c(c1)OC(F)(F)O2] | 3-[[(7R)-1-[6-[[(1S)-1-(2,2-difluoro-1,3- benzodioxol-5-yl)ethyl]amino]-2- pyridyl]-3-(trifluoromethyl)-4,5,6,7- tetrahydroindazol-7- yl]oxy]bicyclo[1.1.1]pentane-1- carboxylic acid |
| 101 (P1) | 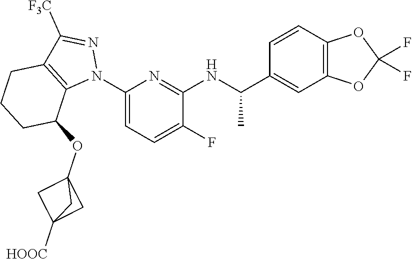 [SMILES: C[C@H](Nc1nc(-n2nc(C(F)(F)F)c3c2[C@@H](OC24CC(C(=O)O)(C2)C4)CCC3)ccc1F)c1ccc2c(c1)OC(F)(F)O2] | 3-[[(7S)-1-[6-[(1S)-1-(2,2-difluoro-1,3- benzodioxol-5-yl)ethyl]amino]-5-fluoro- 2-pyridyl]-3-(trifluoromethyl)-4,5,6,7- tetrahydroindazol-7- yl]oxy]bicyclo[1.1.1]pentane-1- carboxylic acid |
| 101 (P2) | 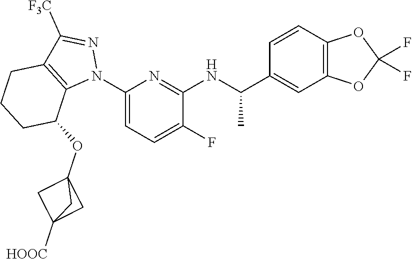 [SMILES: C[C@H](Nc1nc(-n2nc(C(F)(F)F)c3c2[C@H](OC24CC(C(=O)O)(C2)C4)CCC3)ccc1F)c1ccc2c(c1)OC(F)(F)O2] | 3-[[(7R)-1-[6-[[(1S)-1-(2,2-difluoro-1,3- benzodioxol-5-yl)ethyl]amino]-5-fluoro- 2-pyridyl]-3-(trifluoromethyl)-4,5,6,7- tetrahydroindazol-7- yl]oxy]bicyclo[1.1.1]pentane-1- carboxylic acid |
| 102 (P1) | 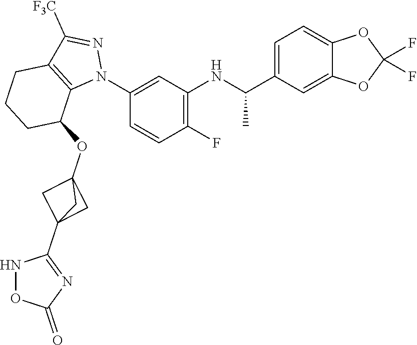 [SMILES: C[C@H](Nc1cc(-n2nc(C(F)(F)F)c3c2[C@@H](OC24CC(c5nc(=O)o[nH]5)(C2)C4)CCC3)ccc1F)c1ccc2c(c1)OC(F)(F)O2] | 3-[3-[[(7S)-1-[3-[[(1S)-1-(2,2-difluoro- 1,3-benzodioxol-5-yl)ethyl]amino]-4- fluoro-phenyl]-3-(trifluoromethyl)- 4,5,6,7-tetrahydroindazol-7-yl]oxy]-1- bicyclo[1.1.1]pentanyl]-2H-1,2,4- oxadiazol-5-one |
| 102 (P2) | 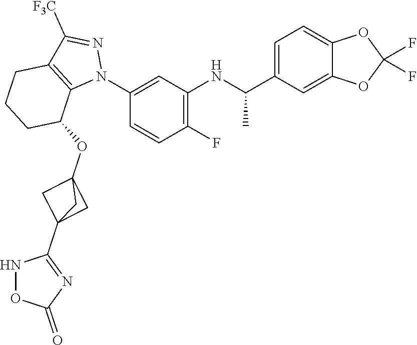 [SMILES: C[C@H](Nc1cc(-n2nc(C(F)(F)F)c3c2[C@H](OC24CC(c5nc(=O)o[nH]5)(C2)C4)CCC3)ccc1F)c1ccc2c(c1)OC(F)(F)O2] | 3-[3-[[(7R)-1-[3-[[(1S)-1-(2,2-difluoro- 1,3-benzodioxol-5-yl)ethyl]amino]-4- fluoro-phenyl]-3-(trifluoromethyl)- 4,5,6,7-tetrahydroindazol-7-yl]oxy]-1- bicyclo[1.1.1]pentanyl]-2H-1,2,4- oxadiazol-5-one |
| 103 (P1) | 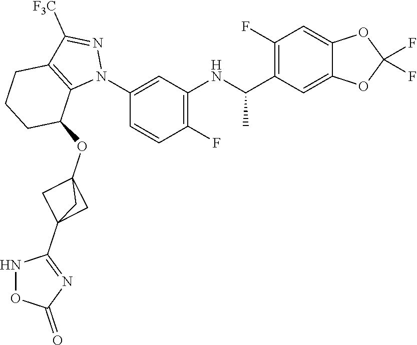 [SMILES: C[C@H](Nc1cc(-n2nc(C(F)(F)F)c3c2[C@@H](OC24CC(c5nc(=O)o[nH]5)(C2)C4)CCC3)ccc1F)c1cc2c(cc1F)OC(F)(F)O2] | 3-[3-[[(7S)-1-[4-fluoro-3-[(1S)-1- (2,2,6-trifluoro-1,3-benzodioxol-5- yl)ethyl]amino]phenyl]-3- (trifluoromethyl)-4,5,6,7- tetrahydroindazol-7-yl]oxy]-1- bicyclo[1.1.1]pentanyl]-2H-1,2,4- oxadiazol-5-one |
| 103 (P2) | 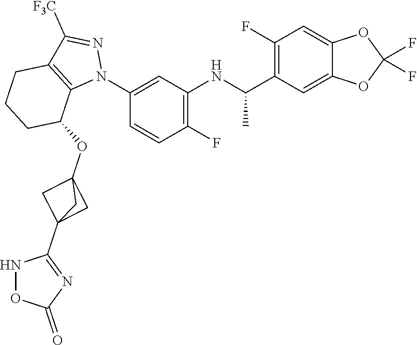 [SMILES: C[C@H](Nc1cc(-n2nc(C(F)(F)F)c3c2[C@H](OC24CC(c5nc(=O)o[nH]5)(C2)C4)CCC3)ccc1F)c1cc2c(cc1F)OC(F)(F)O2] | 3-[3-[[(7R)-1-[4-fluoro-3-[[(1S)-1- (2,2,6-trifluoro-1,3-benzodioxol-5- yl)ethyl]amino]phenyl]-3- (trifluoromethyl)-4,5,6,7- tetrahydroindazol-7-yl]oxy]-1- bicyclo[1.1.1]pentanyl]-2H-1,2,4- oxadiazol-5-one |
| 104 | 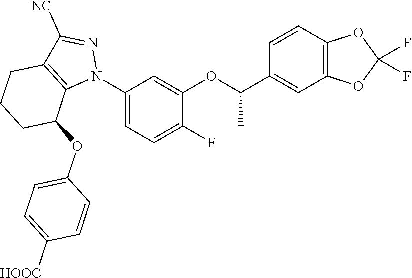 [SMILES: C[C@H](Oc1cc(-n2nc(C#N)c3c2[C@@H](Oc2ccc(C(=O)O)cc2)CCC3)ccc1F)c1ccc2c(c1)OC(F)(F)O2] | 4-[[(7S)-3-cyano-1-[3-[(1S)-1-(2,2- difluoro-1,3-benzodioxol-5-yl)ethoxy]- 4-fluoro-phenyl]-4,5,6,7- tetrahydroindazol-7-yl]oxy]benzoic acid |
| 105 (P1) | 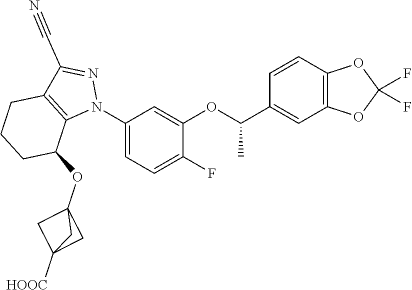 [SMILES: C[C@H](Oc1cc(-n2nc(C#N)c3c2[C@@H](OC24CC(C(=O)O)(C2)C4)CCC3)ccc1F)c1ccc2c(c1)OC(F)(F)O2] | 3-[[(7S)-3-cyano-1-[3-[(1S)-1-(2,2- difluoro-1,3-benzodioxol-5-yl)ethoxy]- 4-fluoro-phenyl]-4,5,6,7- tetrahydroindazol-7- yl]oxy]bicyclo[1.1.1]pentane-1- carboxylic acid |
| 105 (P2) | 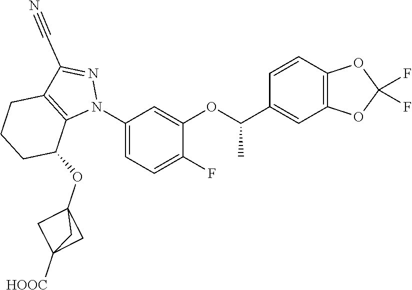 [SMILES: C[C@H](Oc1cc(-n2nc(C#N)c3c2[C@H](OC24CC(C(=O)O)(C2)C4)CCC3)ccc1F)c1ccc2c(c1)OC(F)(F)O2] | 3-[[(7R)-3-cyano-1-[3-[(1S)-1-(2,2- difluoro-1,3-benzodioxol-5-yl)ethoxy]- 4-fluoro-phenyl]-4,5,6,7- tetrahydroindazol-7- yl]oxy]bicyclo[1.1.1]pentane-1- carboxylic acid |
| 106 (P1) | 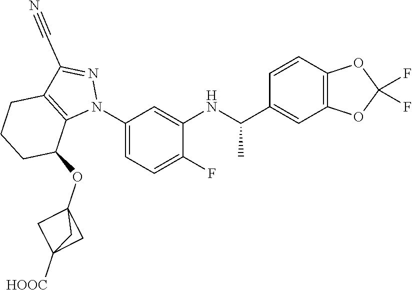 [SMILES: C[C@H](Nc1cc(-n2nc(C#N)c3c2[C@@H](OC24CC(C(=O)O)(C2)C4)CCC3)ccc1F)c1ccc2c(c1)OC(F)(F)O2] | 3-[[(7S)-3-cyano-1-[3-[[(1S)-1-(2,2- difluoro-1,3-benzodioxol-5- yl)ethyl]amino]-4-fluoro-phenyl]- 4,5,6,7-tetrahydroindazol-7- yl]oxy]bicyclo[1.1.1]pentane-1- carboxylic acid |
| 106 (P2) | 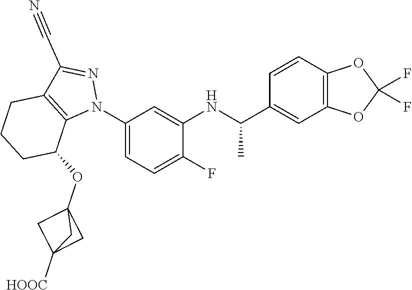 [SMILES: C[C@H](Nc1cc(-n2nc(C#N)c3c2[C@H](OC24CC(C(=O)O)(C2)C4)CCC3)ccc1F)c1ccc2c(c1)OC(F)(F)O2] | 3-[[(7R)-3-cyano-1-[3-[(1S)-1-(2,2- difluoro-1,3-benzodioxol-5- yl)ethyl]amino]-4-fluoro-phenyl]- 4,5,6,7-tetrahydroindazol-7- yl]oxy]bicyclo[1.1.1]pentane-1- carboxylic acid |
| 107 (P1) | 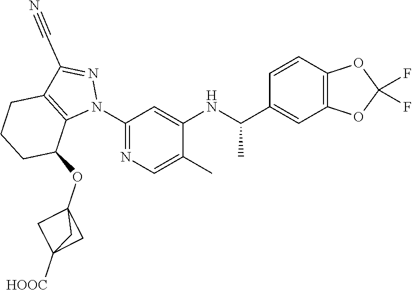 [SMILES: Cc1cnc(-n2nc(C#N)c3c2[C@@H](OC24CC(C(=O)O)(C2)C4)CCC3)cc1N[C@@H](C)c1ccc2c(c1)OC(F)(F)O2] | 3-[[(7S)-3-cyano-1-[4-[[(1S)-1-(2,2- difluoro-1,3-benzodioxol-5- yl)ethyl]amino]-5-fluoro-2-pyridyl]- 4,5,6,7-tetrahydroindazol-7- yl]oxy]bicyclo[1.1.1]pentane-1- carboxylic acid |
| 107 (P2) | 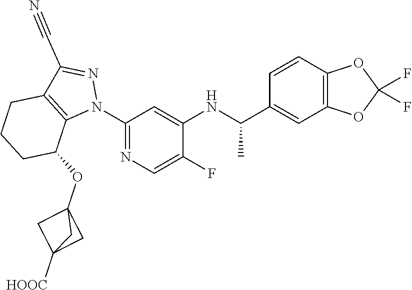 [SMILES: C[C@H](Nc1cc(-n2nc(C#N)c3c2[C@H](OC24CC(C(=O)O)(C2)C4)CCC3)ncc1F)c1ccc2c(c1)OC(F)(F)O2] | 3-[[(7R)-3-cyano-1-[4-[(1S)-1-(2,2- difluoro-1,3-benzodioxol-5- yl)ethyl]amino]-5-fluoro-2-pyridyl]- 4,5,6,7-tetrahydroindazol-7- yl]oxy]bicyclo[1.1.1]pentane-1- carboxylic acid |
| 108 (P1) | 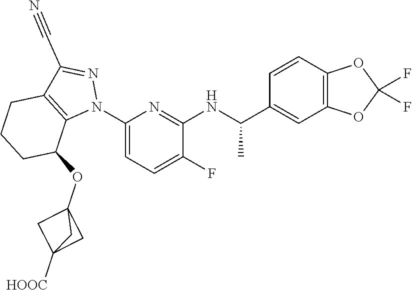 [SMILES: C[C@H](Nc1nc(-n2nc(C#N)c3c2[C@@H](OC24CC(C(=O)O)(C2)C4)CCC3)ccc1F)c1ccc2c(c1)OC(F)(F)O2] | 3-[[(7S)-3-cyano-1-[6-[[(1S)-1-(2,2- difluoro-1,3-benzodioxol-5- yl)ethyl]amino]-5-fluoro-2-pyridyl]- 4,5,6,7-tetrahydroindazol-7- yl]oxy]bicyclo[1.1.1]pentane-1- carboxylic acid |
| 108 (P2) | 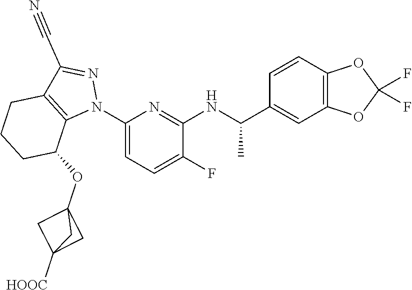 [SMILES: C[C@H](Nc1nc(-n2nc(C#N)c3c2[C@H](OC24CC(C(=O)O)(C2)C4)CCC3)ccc1F)c1ccc2c(c1)OC(F)(F)O2] | 3-[[(7R)-3-cyano-1-[6-[[(1S)-1-(2,2- difluoro-1,3-benzodioxol-5- yl)ethyl]amino]-5-fluoro-2-pyridyl]- 4,5,6,7-tetrahydroindazol-7- yl]oxy]bicyclo[1.1.1]pentane-1- carboxylic acid |
| 109 | 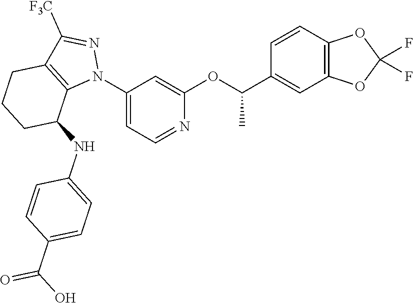 [SMILES: C[C@H](Oc1cc(-n2nc(C(F)(F)F)c3c2[C@@H](Nc2ccc(C(=O)O)cc2)CCC3)ccn1)c1ccc2c(c1)OC(F)(F)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]amino]benzoic acid |
| 110 | 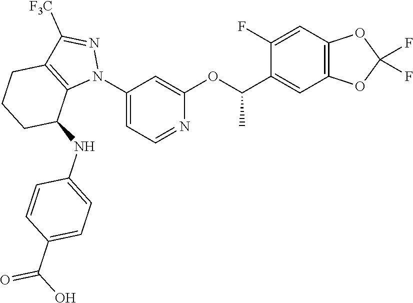 [SMILES: C[C@H](Oc1cc(-n2nc(C(F)(F)F)c3c2[C@@H](Nc2ccc(C(=O)O)cc2)CCC3)ccn1)c1cc2c(cc1F)OC(F)(F)O2] | 4-[[(7S)-1-[2-[(1S)-1-(2,2,6-trifluoro- 1,3-benzodioxol-5-yl)ethoxy]-4- pyridyl]-3-(trifluoromethyl)-4,5,6,7- tetrahydroindazol-7-yl]amino]benzoic acid |
| 111 | 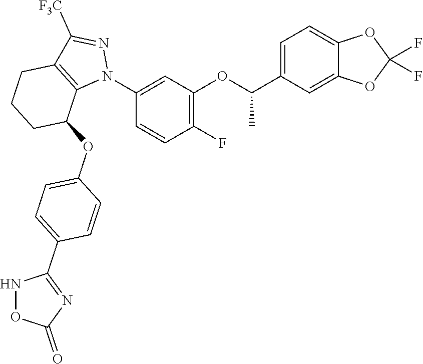 [SMILES: C[C@H](Oc1cc(-n2nc(C(F)(F)F)c3c2[C@@H](Oc2ccc(-c4nc(=O)o[nH]4)cc2)CCC3)ccc1F)c1ccc2c(c1)OC(F)(F)O2] | 3-[4-[[(7S)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]phenyl]-2H- 1,2,4-oxadiazol-5-one |
| 112 | 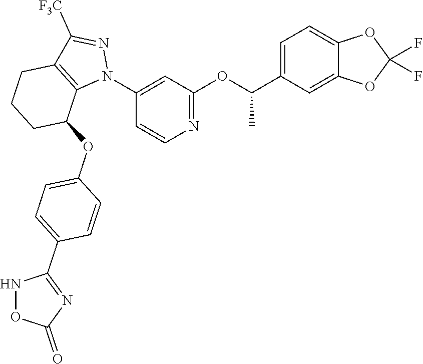 [SMILES: C[C@H](Oc1cc(-n2nc(C(F)(F)F)c3c2[C@@H](Oc2ccc(-c4nc(=O)o[nH]4)cc2)CCC3)ccn1)c1ccc2c(c1)OC(F)(F)O2] | 3-[4-[[(7S)-1-[2-[(1S)-1-(2,2-difluoro- 1,3-benzodioxol-5-yl)ethoxy]-4- pyridyl]-3-(trifluoromethyl)-4,5,6,7- tetrahydroindazol-7-yl]oxy]phenyl]-2H- 1,2,4-oxadiazol-5-one |
| 113 | 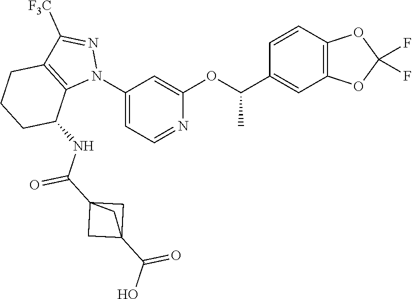 [SMILES: C[C@H](Oc1cc(-n2nc(C(F)(F)F)c3c2[C@H](NC(=O)C24CC(C(=O)O)(C2)C4)CCC3)ccn1)c1ccc2c(c1)OC(F)(F)O2] | 3-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]carbamoyl]bicyclo[1.1.1]pentane-1- carboxylic acid |
| 114 | 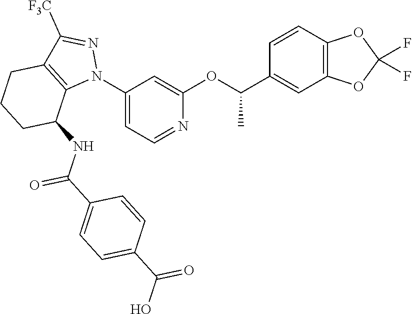 [SMILES: C[C@H](Oc1cc(-n2nc(C(F)(F)F)c3c2[C@@H](NC(=O)c2ccc(C(=O)O)cc2)CCC3)ccn1)c1ccc2c(c1)OC(F)(F)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]carbamoyl]benzoic acid |
| 115 (P1) | 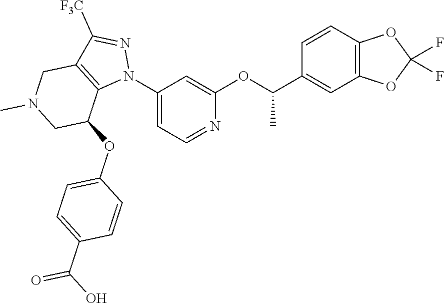 [SMILES: C[C@H](Oc1cc(-n2nc(C(F)(F)F)c3c2[C@@H](Oc2ccc(C(=O)O)cc2)CN(C)C3)ccn1)c1ccc2c(c1)OC(F)(F)O2] | 4-[[(7S)-1-[2-[(1S)-1-(2,2-difluoro-1,3- benzodioxol-5-yl)ethoxy]-4-pyridyl]-5- methyl-3-(trifluoromethyl)-6,7-dihydro- 4H-pyrazolo[4,3-c]pyridin-7- yl]oxy]benzoic acid |
| 115 (P2) | 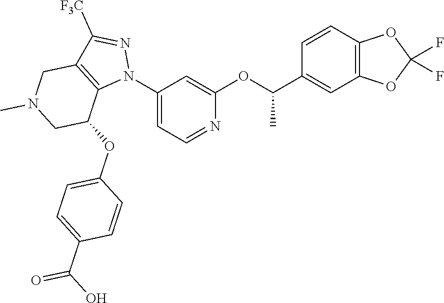 [SMILES: C[C@H](Oc1cc(-n2nc(C(F)(F)F)c3c2[C@H](Oc2ccc(C(=O)O)cc2)CN(C)C3)ccn1)c1ccc2c(c1)OC(F)(F)O2] | 4-[[(7R)-1-[2-[(1S)-1-(2,2-difluoro-1,3- benzodioxol-5-yl)ethoxy]-4-pyridyl]-5- methyl-3-(trifluoromethyl)-6,7-dihydro- 4H-pyrazolo[4,3-c]pyridin-7- yl]oxy]benzoic acid |
| 116 | 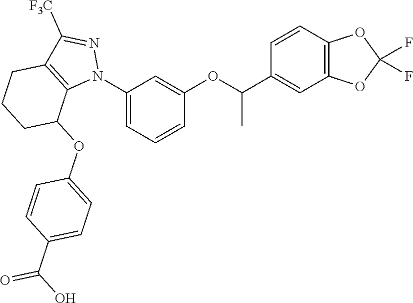 [SMILES: CC(Oc1cccc(-n2nc(C(F)(F)F)c3c2C(Oc2ccc(C(=O)O)cc2)CCC3)c1)c1ccc2c(c1)OC(F)(F)O2] | 4-[[1-[3-[1-(2,2-difluoro-1,3- benzodioxol-5-yl)ethylsulfanyl]phenyl]- 3-(trifluoromethyl)-4,5,6,7- tetrahydroindazol-7-yl]oxy]benzoic acid |
| 117 | 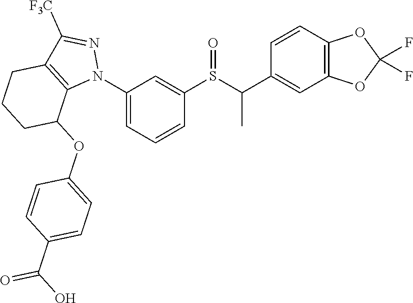 [SMILES: CC(c1ccc2c(c1)OC(F)(F)O2)S(=O)c1cccc(-n2nc(C(F)(F)F)c3c2C(Oc2ccc(C(=O)O)cc2)CCC3)c1] | 4-[[1-[3-[1-(2,2-difluoro-1,3- benzodioxol-5-yl)ethylsulfinyl]phenyl]- 3-(trifluoromethyl)-4,5,6,7- tetrahydroindazol-7-yl]oxy]benzoic acid |
| 118 | 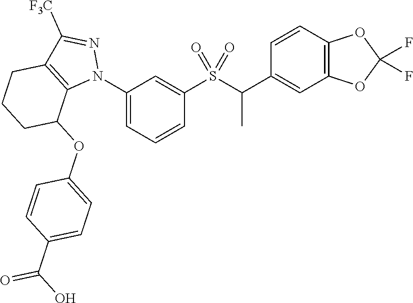 [SMILES: CC(c1ccc2c(c1)OC(F)(F)O2)S(=O)(=O)c1cccc(-n2nc(C(F)(F)F)c3c2C(Oc2ccc(C(=O)O)cc2)CCC3)c1] | 4-[[1-[3-[1-(2,2-difluoro-1,3- benzodioxol-5-yl)ethylsulfonyl]phenyl]- 3-(trifluoromethyl)-4,5,6,7- tetrahydroindazol-7-yl]oxy]benzoic acid |
| 119 | 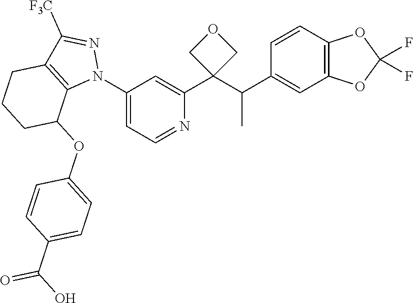 [SMILES: CC(c1ccc2c(c1)OC(F)(F)O2)C1(c2cc(-n3nc(C(F)(F)F)c4c3C(Oc3ccc(C(=O)O)cc3)CCC4)ccn2)COC1] | 4-[[1-[2-[3-[1-(2,2-difluoro-1,3- benzodioxol-5-yl)ethyl]oxetan-3-yl]-4- pyridyl]-3-(trifluoromethyl)-4,5,6,7- tetrahydroindazol-7-yl]oxy]benzoic acid |
| 120 (P1) | 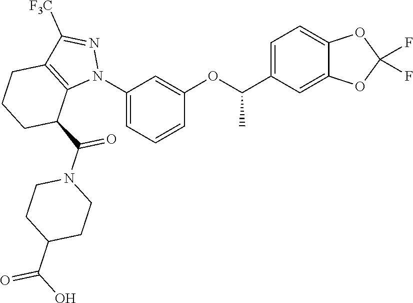 [SMILES: C[C@H](Oc1cccc(-n2nc(C(F)(F)F)c3c2[C@@H](C(=O)N2CCC(C(=O)O)CC2)CCC3)c1)c1ccc2c(c1)OC(F)(F)O2] | 1-[(7S)-1-[3-[(1S)-1-(2,2-difluoro-1,3- benzodioxol-5-yl)ethoxy]phenyl]-3- (trifluoromethyl)-4,5,6,7- tetrahydroindazole-7- carbonyl]piperidine-4-carboxylic acid |
| 120 (P2) | 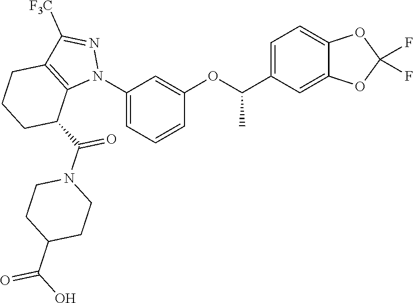 [SMILES: C[C@H](Oc1cccc(-n2nc(C(F)(F)F)c3c2[C@H](C(=O)N2CCC(C(=O)O)CC2)CCC3)c1)c1ccc2c(c1)OC(F)(F)O2] | 1-[(7R)-1-[3-[(1S)-1-(2,2-difluoro-1,3- benzodioxol-5-yl)ethoxy]phenyl]-3- (trifluoromethyl)-4,5,6,7- tetrahydroindazole-7- carbonyl]piperidine-4-carboxylic acid |
| 121 (P1) | 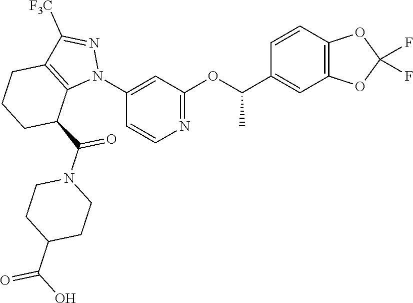 [SMILES: C[C@H](Oc1cc(-n2nc(C(F)(F)F)c3c2[C@@H](C(=O)N2CCC(C(=O)O)CC2)CCC3)ccn1)c1ccc2c(c1)OC(F)(F)O2] | 1-[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazole-7- carbonyl]piperidine-4-carboxylic acid |
| 121 (P2) | 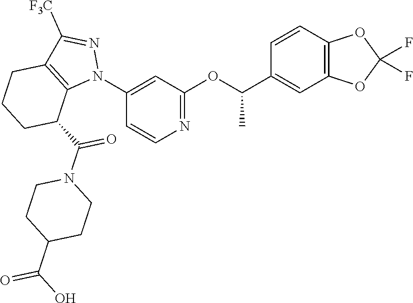 [SMILES: C[C@H](Oc1cc(-n2nc(C(F)(F)F)c3c2[C@H](C(=O)N2CCC(C(=O)O)CC2)CCC3)ccn1)c1ccc2c(c1)OC(F)(F)O2] | 1-[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazole-7- carbonyl]piperidine-4-carboxylic acid |
| 122 (P1) | 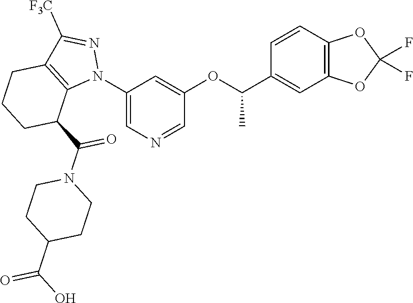 [SMILES: C[C@H](Oc1cncc(-n2nc(C(F)(F)F)c3c2[C@@H](C(=O)N2CCC(C(=O)O)CC2)CCC3)c1)c1ccc2c(c1)OC(F)(F)O2] | 1-[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazole-7- carbonyl]piperidine-4-carboxylic acid |
| 122 (P2) | 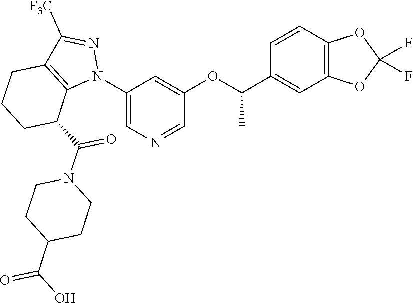 [SMILES: C[C@H](Oc1cncc(-n2nc(C(F)(F)F)c3c2[C@H](C(=O)N2CCC(C(=O)O)CC2)CCC3)c1)c1ccc2c(c1)OC(F)(F)O2] | 1-[(7R)-1-[5-[(1S)-1-(2,2-difluoro-1,3- benzodioxol-5-yl)ethoxy]-3-pyridyl]-3- (trifluoromethyl)-4,5,6,7- tetrahydroindazole-7- carbonyl]piperidine-4-carboxylic acid |
| 123 | 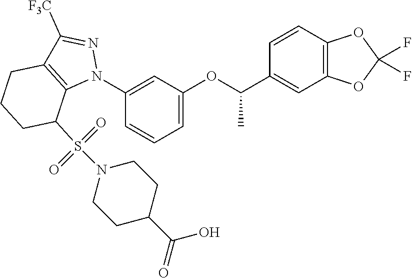 [SMILES: C[C@H](Oc1cccc(-n2nc(C(F)(F)F)c3c2C(S(=O)(=O)N2CCC(C(=O)O)CC2)CCC3)c1)c1ccc2c(c1)OC(F)(F)O2] | 1-[[1-[3-[(1S)-1-(2,2-difluoro-1,3- benzodioxol-5-yl)ethoxy]phenyl]-3- (trifluoromethyl)-4,5,6,7- tetrahydroindazol-7- yl]sulfonyl]piperidine-4-carboxylic acid |
| 124 (P1) | 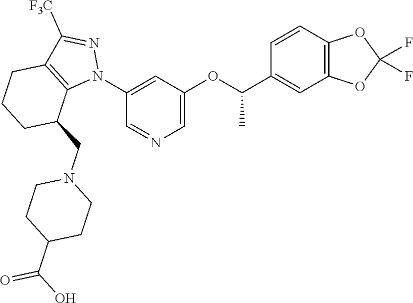 [SMILES: C[C@H](Oc1cncc(-n2nc(C(F)(F)F)c3c2[C@@H](CN2CCC(C(=O)O)CC2)CCC3)c1)c1ccc2c(c1)OC(F)(F)O2] | 1-[[(7R)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl]methyl]piperidine-4-carboxylic acid |
| 124 (P2) | 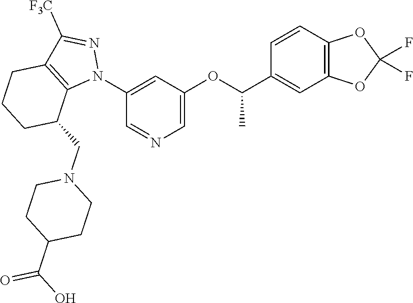 [SMILES: C[C@H](Oc1cncc(-n2nc(C(F)(F)F)c3c2[C@H](CN2CCC(C(=O)O)CC2)CCC3)c1)c1ccc2c(c1)OC(F)(F)O2] | 1-[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl]methyl]piperidine-4-carboxylic acid |
| 125 (P1) | 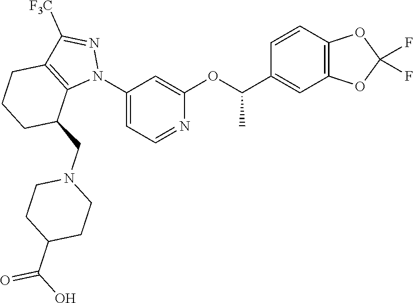 [SMILES: C[C@H](Oc1cc(-n2nc(C(F)(F)F)c3c2[C@@H](CN2CCC(C(=O)O)CC2)CCC3)ccn1)c1ccc2c(c1)OC(F)(F)O2] | 1-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]methyl]piperidine-4-carboxylic acid |
| 125 (P2) | 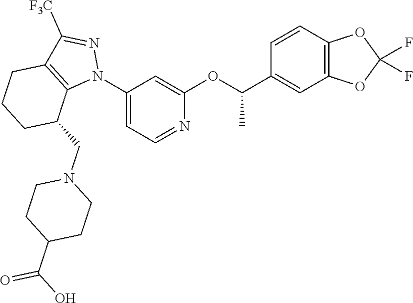 [SMILES: C[C@H](Oc1cc(-n2nc(C(F)(F)F)c3c2[C@H](CN2CCC(C(=O)O)CC2)CCC3)ccn1)c1ccc2c(c1)OC(F)(F)O2] | 1-[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]methyl]piperidine-4-carboxylic acid |
| 126 | 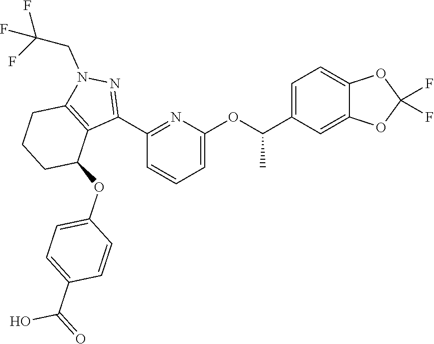 [SMILES: C[C@H](Oc1cccc(-c2nn(CC(F)(F)F)c3c2[C@@H](Oc2ccc(C(=O)O)cc2)CCC3)n1)c1ccc2c(c1)OC(F)(F)O2] | 4-[[(4S)-3-[6-[(1S)-1-(2,2-difluoro-1,3- benzodioxol-5-yl)ethoxy]-2-pyridyl]-1- (2,2,2-trifluoroethyl)-4,5,6,7- tetrahydroindazol-4-yl]oxy]benzoic acid |
| 127 | 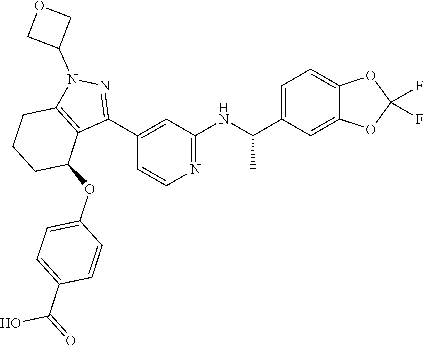 [SMILES: C[C@H](Nc1cc(-c2nn(C3COC3)c3c2[C@@H](Oc2ccc(C(=O)O)cc2)CCC3)ccn1)c1ccc2c(c1)OC(F)(F)O2] | 4-[[(4S)-3-[2-[[(1S)-1-(2,2-difluoro-1,3- benzodioxol-5-yl)ethyl]amino]-4- pyridyl]-1-(oxetan-3-yl)-4,5,6,7- tetrahydroindazol-4-yl]oxy]benzoic acid |
| 128 | 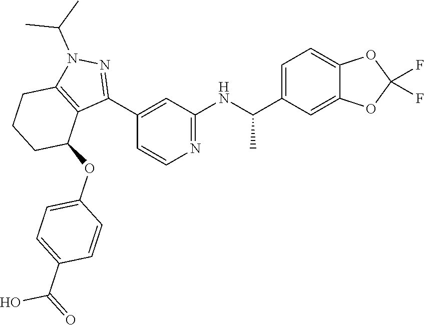 [SMILES: CC(C)n1nc(-c2ccnc(N[C@@H](C)c3ccc4c(c3)OC(F)(F)O4)c2)c2c1CCC[C@@H]2Oc1ccc(C(=O)O)cc1] | 4-[[(4S)-3-[2-[[(1S)-1-(2,2-difluoro-1,3- benzodioxol-5-yl)ethyl]amino]-4- pyridyl]-1-isopropyl-4,5,6,7- tetrahydroindazol-4-yl]oxy]benzoic acid |
| 129 | 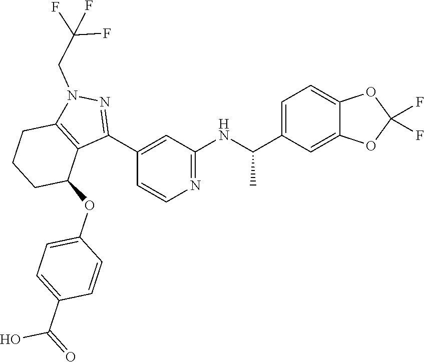 [SMILES: C[C@H](Nc1cc(-c2nn(CC(F)(F)F)c3c2[C@@H](Oc2ccc(C(=O)O)cc2)CCC3)ccn1)c1ccc2c(c1)OC(F)(F)O2] | 4-[[(4S)-3-[2-[[(1S)-1-(2,2-difluoro-1,3- benzodioxol-5-yl)ethyl]amino]-4- pyridyl]-1-(2,2,2-trifluoroethyl)-4,5,6,7- tetrahydroindazol-4-yl]oxy]benzoic acid |

or a pharmaceutically acceptable salt thereof.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “carbocyclyl”) refers to a monocyclic or bicyclic C₃-C₁₀ hydrocarbon that is completely saturated or that contains one or more units of unsaturation, but which is not fully aromatic, that has a single point of attachment to the rest of the molecule. The terms “cycloaliphatic”, “carbocycle”, and “carbocyclyl” are used interchangeably herein, and also include groups in which a carbocyclyl ring is fused to one or more cycloaliphatic rings. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “haloalkyl” refers to a straight or branched alkyl group that is substituted with one or more halogen atoms.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C₆). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group. The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used, herein, is “group in which an aromatic ring is fused to one or “ore” on-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted, including groups in which a heteroaryl ring is fused to one or more aryl, heteroaryl, or cycloaliphatic rings. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O) SR^{∘}; —(CH₂)₀₋₄C(O)OsiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘)}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O (haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OsiR^{•}₃, —C(O) SR^{•}, —(C₁₋₄ straight or branched alkylene) C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{•} include halogen, —R^{•}, -(haloR^{•}), —OH, —OR^{•}, —O (haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†}) S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O (haloR^{•}), —CN, —C(O)OH, —C(O)OR^{•}, —NH₂, —NHR′, —NR^{∘}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl) 4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat and/or diagnose the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an CFTR-associated disease or disorder.

The terms “treat”, “treatment” or “treating” mean to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, “CFTR” means cystic fibrosis transmembrane conductance regulator. Defects in the function of the CFTR ion channel result from loss of function mutations of CFTR. Such mutations lead to exocrine gland dysfunction, abnormal mucociliary clearance, and cause cystic fibrosis. The most common CFTR mutation in Cystic Fibrosis (CF) patients leads to the specific deletion of three nucleotides of the codon for phenylalanine at position 508. This mutation, which is found in ˜70% of CF patients worldwide, is referred to as “ΔF508”. The ΔF508 mutation decreases the stability of the CFTR NBD1 domain and limits CFTR interdomain assembly. Since CF is an autosomal recessive disease, a CF patient harboring the ΔF508 CFTR mutation must also carry a second defective copy of CFTR. Approximately 2000 different CF-causing CFTR mutations have been identified in CF patients. CF patients harboring the ΔF508 CFTR mutation can be homozygous for that mutation (ΔF508/ΔF508). CF patients can also be ΔF508 heterozygous, if the second CFTR allele such patients carry instead contains a different CFTR loss of function mutation. Such CFTR mutations include, but are not limited to, G542X, G551D, N1303K, W1282X, R553X, R117H, R1162X, R347P, G85E, R560T, A455E, ΔI507, G178R, S549N, S549R, G551S, G970R, G1244E, S1251N, S1255P, and G1349D.

As used herein, the term “CFTR modulator” refers to a compound that increases the activity of CFTR. In certain aspects, a CFTR modulator is a CFTR corrector or a CFTR potentiator or a dual-acting compound having activities of a corrector and a potentiator.

As used herein, the term “CFTR corrector” refers to a compound that increases the amount of functional CFTR protein to the cell surface and thus enhances CFTR channel function. The CFTR correctors partially “rescue” misfolding of CFTR, thereby enabling the maturation and functional expression of CFTR protein harboring a CF causing mutation on the cell surface. Examples of correctors include, but are not limited to, VX-809, VX-661, VX-152, VX-440, VX-983, and GLPG2222. Such compounds may interact directly with CFTR protein, modifying its folding and conformational maturation during synthesis.

As used herein, the term “CFTR potentiator” refers to a compound that increases the ion channel activity of CFTR protein located at the cell surface, resulting in enhanced ion transport. CFTR potentiators repair the defective channel functions caused by mutations. Examples of potentiators include, but are not limited to, ivacaftor (VX770), deuterated ivacaftor (CPT 656), genistein and GLPG1837.

As used herein, the term “CFTR pharmacological chaperone” (PC) refers to compounds that stabilize the CFTR protein in its native state by binding directly to the protein.

As used herein, the term “CFTR proteostasis regulator” (PR) refers to compounds that enhance the protein folding efficiency within the cell. PRs can alter the activity of transcriptional, folding and/or membrane trafficking machinery, as well as impeding the degradation of partially folded, but functional, conformers at the endoplasmic reticulum (ER) or plasma membrane.

As used herein, “CFTR disease or condition” refers to a disease or condition associated with deficient CFTR activity, for example, cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, A-beta.-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably modulate CFTR, or a mutant thereof, in a biological sample or in a patient. In certain embodiments, the amount of compound in compositions of this disclosure is such that is effective to measurably modulate CFTR, or a mutant thereof, in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, the amount of compound in compositions contemplated herein is such that is effective to measurably modulate a protein, particularly at CFTR, or a mutant thereof, in a biological sample or in a patient. In certain embodiments, the amount of compound in compositions of this disclosure is such that is effective to measurably modulate CFTR, or a mutant thereof, in a biological sample or in a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (A) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (A) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (A) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (A) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (A) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

As discussed above, CFTR is composed of two six membrane-spanning domains (MSD1 and MSD2), two nucleotide bind domains (NBD1 and NBD2), a regulatory region I and four cytosolic loops (CL1-4). CFTR protein is located primarily in the apical membrane of epithelial cells where it functions to conduct anions, including chloride, bicarbonate and thiocyanate into and out of the cell. The most frequent CFTR mutation is the in-frame deletion of phenylalanine at residue 508 (ΔF508) in the first nucleotide binding domain (NBD1). The mutation has several deleterious effects on the production of CFTR in the ER, its correct folding, its movement to the plasma membrane and its normal function as an ion channel for the cell.

One such negative effect is that the NBD1 domain is partially or mis-folded which is recognized within the cell as an aberrant protein and tagged for disposal by ER-associated degradation (ERAD) via the ubiquitin-proteasome system (UPS). Should a partially or mis-folded CFTR protein emerge from the ER, the protein must travel to the plasma membrane through complex glycosylation in the Golgi compartment and be functionally inserted. In wild-type CFTR, only 20-40% of CFTR reaches the plasma membrane, indicating that CFTR has energetic instability of individual NBDs, a slow domain assembly, and relatively fast ERAD kinetics which all contribute to inefficient folding and sensitize CFTR to structural perturbations by mutations.

In wild-type CFTR, the NBD1 domain folds co-translationally while other domains fold post-translationally. Mutated ΔF508 CFTR has impaired NBD1 folding but its backbone structure and thermodynamic stability are similar to wild-type CFTR. With delayed folding kinetics, mutated ΔF508 CFTR NBD1 has an increased folding activation energy. Lack of proper folding results in hydrophobic residues being exposed to the surface of NBD1 which causes aggregation with other CFTR proteins. Thus, the aggregation temperature of mutated CFTR drops from 41° C. to 33° C. This level of instability creates a greater percentage of mis-folded mutant CFTR at physiological temperature (37° C. in humans). Mutant CFTR suffers from both kinetic and thermodynamic folding defects. CFTR stabilizers can address these folding defects, but complete energetic correction of mutant NBD1 folding has been shown to not result in the CFTR biosynthetic processing, underscoring the need for interface stability as well.

The disclosed CFTR correctors can interact with the NBD domain to stabilize the correct folded position R, such that CFTR is not labeled for elimination from the cell. The preservation of correct folding enables CFTR to function as a chloride ion channel at wild-type levels. In some embodiments, disclosed CFTR correctors can enhance the performance of wild-type CFTR.

CFTR stabilizers can function in combination with other therapeutic agents such as CFTR correctors that promote A508 CFTR exit from the ER and accumulation in the plasma membrane. Increasing the amount of CFTR cell surface expression can result in improved chloride conductance following channel activation by both potentiators and a cAMP agonist. Thus, disclosed herein are combinations of CFTR stabilizers with CFTR correctors and potentiators, optionally with cAMP agonists or another therapeutic agent as described below.

Disclosed herein are methods of treating deficient CFTR activity in a cell, comprising contacting the cell with a compound of Formula (A), or a pharmaceutically acceptable salt thereof. In certain embodiments, contacting the cell occurs in a subject in need thereof, thereby treating a disease or disorder mediated by deficient CFTR activity.

Also, disclosed herein are methods of treating a disease or a disorder mediated by deficient CFTR activity comprising administering a compound of Formula (A) or a pharmaceutically acceptable salt thereof. In some embodiments, the subject is a mammal, preferably a human. In some embodiments, the disease is associated with the regulation of fluid volumes across epithelial membranes, particularly an obstructive airway disease such as CF or COPD.

Such diseases and conditions include, but are not limited to, cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type 1, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, the disease is cystic fibrosis.

Provided herein are methods of treating cystic fibrosis, comprising administering to a subject in need thereof, a compound as disclosed herein or a pharmaceutically acceptable salt thereof. Also provided herein are methods of lessening the severity of cystic fibrosis, comprising administering to a subject in need thereof, a compound as disclosed herein or a pharmaceutically acceptable salt thereof. In some embodiments, the subject is a human. In some embodiments, the subject is at risk of developing cystic fibrosis, and administration is carried out prior to the onset of symptoms of cystic fibrosis in the subject.

Provided herein are compounds as disclosed herein for use in treating a disease or condition mediated by deficient CFTR activity. Also provided herein are uses of a compound as disclosed herein for the manufacture of a medicament for treating a disease or condition mediated by deficient CFTR activity.

Provided herein are kits for use in measuring the activity of CFTR or a fragment thereof in a biological sample in vitro or in vivo. The kit can contain: (i) a compound as disclosed herein, or a pharmaceutical composition comprising the disclosed compound, and (ii) instructions for: a) contacting the compound or composition with the biological sample; and b) measuring activity of said CFTR or a fragment thereof. In some embodiments, the biological sample is biopsied material obtained from a mammal or extracts thereof; blood, saliva, urine, feces, semen, tears, other body fluids, or extracts thereof. In some embodiments, the mammal is a human.

### Combination Treatments

As used herein, the term “combination therapy” means administering to a subject (e.g., human) two or more CFTR modulators, or a CFTR modulator and an agent such as antibiotics, EnaC inhibitors, GSNO (S-nitrosothiol, s-nitroglutathione) reductase inhibitors, and a CRISPR Cas correction therapy or system (as described in US 2007/0022507 and the like). In some embodiments, combination therapy includes administration of a compound described herein with a compound that modulates CFTR protein or ABC protein activities (e.g., as described in WO2018167690A1 and the like).

In certain embodiments, the method of treating a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

In certain embodiments, the method of preventing a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

Additional therapeutic agents include, for example, EnaC inhibitors, mucolytic agents, modulators of mucus rheology, bronchodilators, antibiotics, anti-infective agents, anti-inflammatory agents, ion channel modulating agents, therapeutic agents used in gene or mRNA therapy, agents that reduce airway surface liquid and/or reduce airway surface PH, CFTR correctors, and CFTR potentiators, or other agents that modulate CFTR activity. Other therapeutics include liposomal composition components such as those described in WO2012/170889, hybrid oligonucleotides that facilitate RNA cleavage such as those described in WO2016/130943, and single stranded oligonucleotides that modulate gene expression as described in WO2016/130929.

In some embodiments, at least one additional therapeutic agent is selected from one or more CFTR modulators, one or more CFTR correctors and one or more CFTR potentiators.

Non-limiting examples of additional therapeutics include VX-770 (Ivacaftor), VX-809 (Lumacaftor, 3-(6-(1-(2,2-difluorobenzo[d][1,3]dioxol-5-yl) cyclopropane-1-carboxamido)-3-methylpyridin-2-yl)benzoic acid, VX-661 (Tezacaftor, (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl) cyclopropane-1-carboxamide, VX-983, VX-152, VX-440, VX-445, VX-659, VX-371, Orkambi, Ataluren (PTC 124) (3-[5-(2-fluorophenyl)-1, 2,4-oxadiazol-3-yl]benzoic acid), PTI-130 (Proteostasis), PTI-801, PTI-808, PTI-428, N91115.74 (cavosonstat), QBW251 (Novartis) compounds described in WO2011113894, compounds N30 Pharmaceuticals (e.g., WO 2014/186704), deuterated ivacaftor (e.g., CTP-656 or VX-561), GLPG 2222, GLPG2451, GLPG3067, GLPG2851, GLPG2737, GLPG 1837 (N-(3-carbamoyl-5,5,7,7-tetramethyl-5,7-dihydro-4H-thieno[2,3-c]pyran-2-yl)-1H-pyrazole-5-carboxamide), GLPG 2665 (Galapagos), FDL 169 (Flatley Discovery lab), FDL 176, FDL438, FDL304, FD2052160, FD1881042, FD2027304, FD2035659, FD2033129, FD1860293, CFFT-Pot01, CFFT-Pot-02, P-1037, glycerol, phenylbutyrate, and the like.

Non-limiting examples of anti-inflammatory agents are N6022 3-(5-(4-(1H-imidazol-1-yl)phenyl)-1-(4-carbamoyl-2-methylphenyl)-1H-pyrrol-2-yl) propanoic acid), Ibuprofen, Lenabasum (anabasum), Acebilustat (CTX-4430), LAU-7b, POL6014, docosahexaenoic acid, alpha-1 anti-trypsin, sildenafil. Additional therapeutic agents also include, but are not limited to a mucolytic agent, a modifier of mucus rheology (such as hypertonic saline, mannitol, and oligosaccharide based therapy), a bronchodilator, an anti-infective (such as tazobactam, piperacillin, rifampin, meropenem, ceftazidime, aztreonam, tobramycin, fosfomycin, azithromycin, amitriptyline, vancomycin, gallium and colistin), an anti-infective agent, an anti-inflammatory agent, a CFTR modulator other than a compound of the present disclosure, and a nutritional agent. Additional therapeutic agents can include treatments for comorbid conditions of cystic fibrosis, such as exocrine pancreatic insufficiency which can be treated with Pancrelipase or Liprotamase.

Examples of CFTR potentiators include, but are not limited to, Ivacaftor (VX-770), CTP-656, NVS-QBW251, FD1860293, GLPG2451, GLPG1837, and N-(3-carbamoyl-5,5,7,7-tetramethyl-5,7-dihydro-4H-thieno[2,3-c]pyran-2-yl)-1H-pyrazole-5-carboxamide. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014180562, WO2015018823, and U.S. patent application Ser. Nos. 14/271,080, 14/451,619 and 15/164,317.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661), VX-983, GLPG2222, GLPG2665, GLPG2737, VX-152, VX-440, FDL169, FDL304, FD2052160, and FD2035659. Examples of correctors are also disclosed in US20160095858A1, and U.S. application Ser. Nos. 14/925,649 and 14/926,727.

In certain embodiments, the additional therapeutic agent is a CFTR amplifier. CFTR amplifiers enhance the effect of known CFTR modulators, such as potentiators and correctors. Examples of CFTR amplifier include PTI130 and PTI-428. Examples of amplifiers are also disclosed in publications: WO2015138909 and WO2015138934.

In certain embodiments, the additional therapeutic agent is an agent that reduces the activity of the epithelial sodium channel blocker (EnaC) either directly by blocking the channel or indirectly by modulation of proteases that lead to an increase in EnaC activity (e.g., serine proteases, channel-activating proteases). Exemplary of such agents include camostat (a trypsin-like protease inhibitor), QAU145, 552-02, GS-9411, INO-4995, Aerolytic, amiloride, AZD5634, and VX-371. Additional agents that reduce the activity of the epithelial sodium channel blocker (EnaC) can be found, for example, in PCT Publication No. WO2009074575 and WO2013043720; and U.S. Pat. No. 8,999,976.

In one embodiment, the EnaC inhibitor is VX-371.

In one embodiment, the EnaC inhibitor is SPX-101 (S18).

### Enumerated Embodiments

The present disclosure includes enumerated embodiments 1-30:
1. A compound represented by Formula (I):

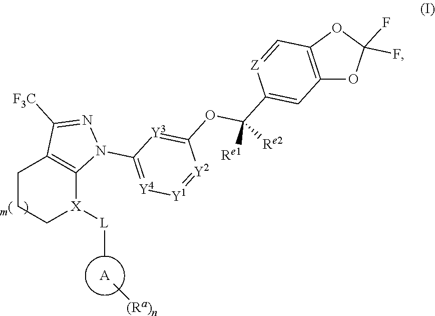

or a pharmaceutically acceptable salt thereof
A is selected from the group consisting of optionally substituted 3-10 membered carbocyclyl, optionally substituted 3-10 membered heterocyclyl, optionally substituted aryl, and optionally substituted 5-10 membered heteroaryl;
L is selected from the group consisting of a bond, —O—, —S—, —S(O)₂—, —CH₂—, —C(O)—, and —N(H)—;
X is N or C;
Y¹ is selected from the group consisting of —C(H)═, —C(F)═, and —N═;
Y² is selected from the group consisting of —C(H)═, —C(F)═, and —N═;
Y³ is selected from the group consisting of —C(H)═, —C(F)═, and —N═;
Y⁴ is selected from the group consisting of —C(H)═, —C(F)═, and —N═;
Z is selected from the group consisting of —C(H)═, —C(F)═, and —N═;
each R^{a} is independently selected from the group consisting of halogen, optionally substituted C₁-C₃ aliphatic, and —COOH;
R^{e1} is selected from the group consisting of hydrogen, C₁-C₅-alkyl, and C₃-C₅-cycloalkyl;
R^{e2} is selected from the group consisting of hydrogen, C₁-C₅-alkyl, and C₃-C₅-cycloalkyl;
m is 0-2; and
n is 0-5.
2. The compound of embodiment 1, wherein the compound is represented by (I-a) or (I-b)

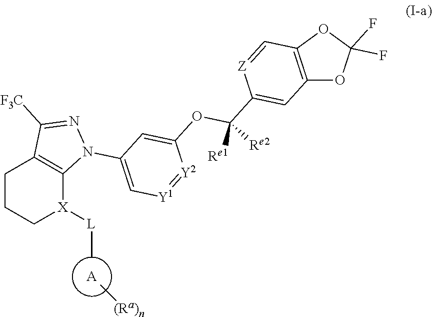

or a pharmaceutically acceptable salt thereof.
3. The compound of embodiment 1, wherein the compound is represented by Formula (I-b1) or (I-b2)

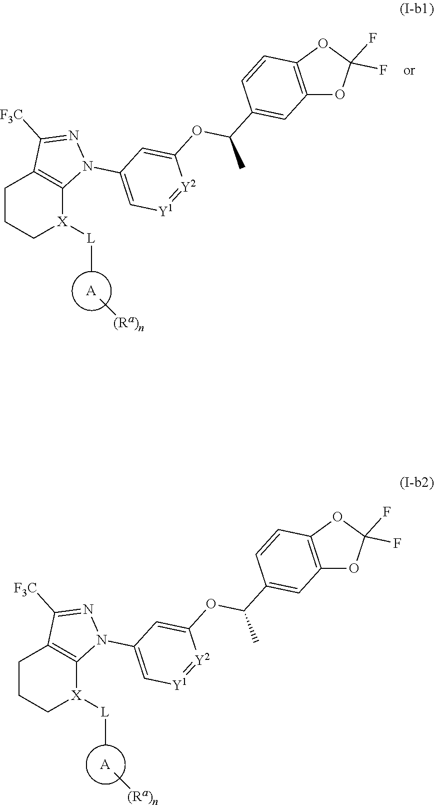

or a pharmaceutically acceptable salt thereof.
4. The compound of any of embodiments 1-3, wherein X is C.
5. The compound of any of embodiments 1-4, wherein L is —O—.
6. The compound of any of embodiments 1-3, wherein X is N.
7. The compound of any of embodiments 1-6, wherein L is selected from the group consisting of —S(O)₂—, —CH₂—, and —C(O)—.
8. The compound of any of embodiments 1-6, wherein L is —CH₂—.
9. The compound of any of embodiments 1-8, wherein A is selected from the group consisting of optionally substituted 5-8 membered carbocyclyl, optionally substituted 5-7 membered heterocyclyl, and optionally substituted phenyl.
10. The compound of any of embodiments 1-8, wherein A is selected from the group consisting of optionally substituted cyclohexyl, optionally substituted bicyclo[1.1.1]pentyl, optionally substituted bicyclo[2.2.2]octyl, optionally substituted piperidinyl, and optionally substituted phenyl.
11. The compound of any of embodiments 1-8, wherein A is selected from the group consisting of

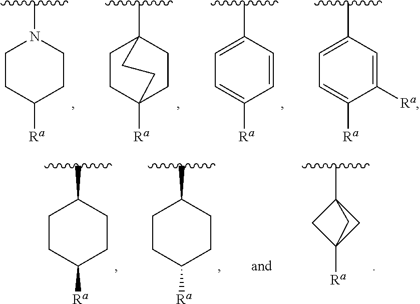

12. The compound of any of embodiments 1-11, wherein each R^{a} is independently selected from the group consisting of methyl and —COOH.
13. The compound of any of embodiments 1-12, wherein n is 1-5 and at least one instance of R^{a} is —COOH.
14. The compound of embodiment 11, wherein R^{a} is —COOH.
15. The compound of any of embodiments 1-14, wherein Y¹ is —C(H)═ or —N═, and Y² is —C(H)═ or —N═.
16. The compound of any of embodiments 1-14, wherein Y¹ is —C(H)═ and Y² is —C(H)═.
17. The compound of any of embodiments 1-14, wherein Y¹ is —N═ and Y² is —C(H)═.
18. The compound of any of embodiments 1-14, wherein Y¹ is —C(H)═ and Y² is —N═.
19. The compound of any of embodiments 1-14, wherein Y¹ is —N═ and Y² is —N═.
20. The compound of any of embodiments 1-19, wherein Z is —C(H)═.
21. The compound of any of embodiments 1-19, wherein Z is —N═.
22. The compound of any of embodiments 1-19, wherein Z is —C(F)—.
23. A compound selected from the group consisting of

| Ex. No. | Structure | IUPAC name |
|---|---|---|
| 1 | 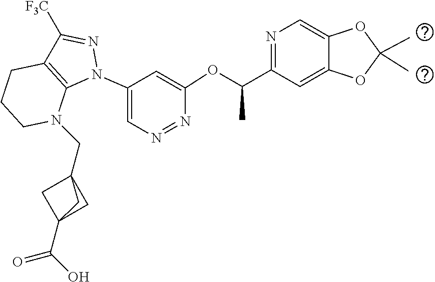 [SMILES: C[C@@H](Oc1cc(-n2nc(C(F)(F)F)c3c2N(CC24CC(C(=O)O)(C2)C4)CCC3)cnn1)c1cc2c(cn1)OC(C)(C)O2] | 3-[[1-[6-[(1R)-1-(2,2-difluoro- [1,3]dioxolo[4,5-c]pyridin-6- yl)ethoxy]pyridazin-4-yl]-3- (trifluoromethyl)-5,6-dihydro-4H- pyrazolo[3,4-b]pyridin-7- yl]methyl]bicyclo[1.1.1]pentane-1- carboxylic acid |
| 2 | 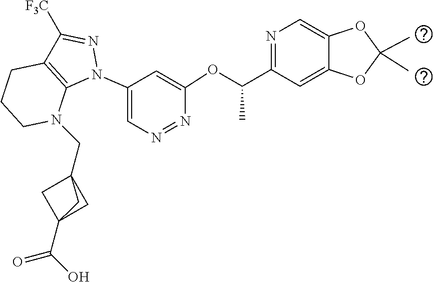 [SMILES: C[C@H](Oc1cc(-n2nc(C(F)(F)F)c3c2N(CC24CC(C(=O)O)(C2)C4)CCC3)cnn1)c1cc2c(cn1)OC(C)(C)O2] | 3-[[1-[6-[(1S)-1-(2,2-difluoro- [1,3]dioxolo[4,5-c]pyridin-6- yl)ethoxy]pyridazin-4-yl]-3- (trifluoromethyl)-5,6-dihydro-4H- pyrazolo[3,4-b]pyridin-7- yl]methyl]bicyclo[1.1.1]pentane-1- carboxylic acid |
| 3 | 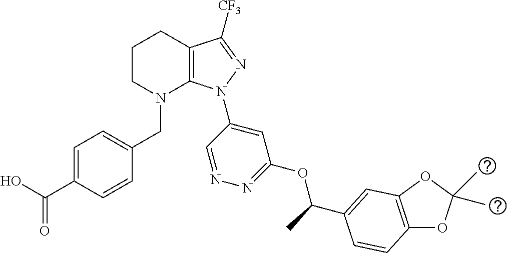 [SMILES: C[C@@H](Oc1cc(-n2nc(C(F)(F)F)c3c2N(Cc2ccc(C(=O)O)cc2)CCC3)cnn1)c1ccc2c(c1)OC(C)(C)O2] | 4-[[1-[6-[(1R)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]benzoic acid |
| 4 | 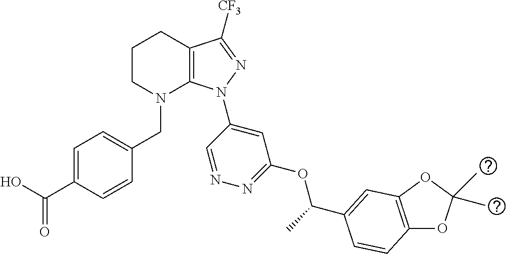 [SMILES: C[C@H](Oc1cc(-n2nc(C(F)(F)F)c3c2N(Cc2ccc(C(=O)O)cc2)CCC3)cnn1)c1ccc2c(c1)OC(C)(C)O2] | 4-[[1-[6-[(1S)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]benzoic acid |
| 5 | 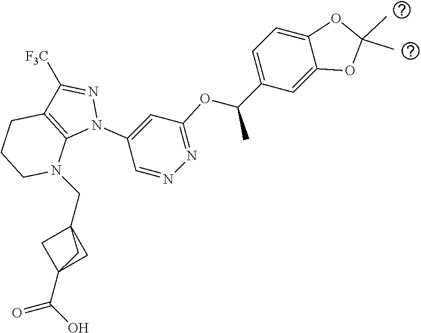 [SMILES: C[C@@H](Oc1cc(-n2nc(C(F)(F)F)c3c2N(CC24CC(C(=O)O)(C2)C4)CCC3)cnn1)c1ccc2c(c1)OC(C)(C)O2] | 3-[[1-[6-[(1R)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]bicyclo[1.1.1]pentane-1- carboxylic acid |
| 6 | 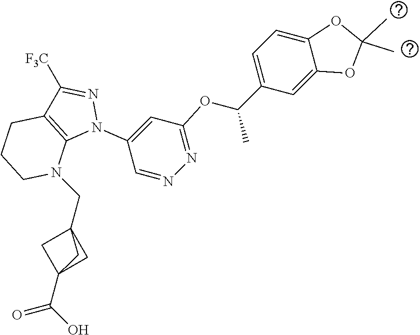 [SMILES: C[C@H](Oc1cc(-n2nc(C(F)(F)F)c3c2N(CC24CC(C(=O)O)(C2)C4)CCC3)cnn1)c1ccc2c(c1)OC(C)(C)O2] | 3-[[1-[6-[(1S)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]bicyclo[1.1.1]pentane-1- carboxylic acid |
| 7 | 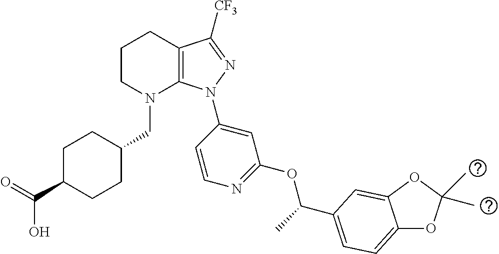 [SMILES: C[C@H](Oc1cc(-n2nc(C(F)(F)F)c3c2N(C[C@H]2CC[C@H](C(=O)O)CC2)CCC3)ccn1)c1ccc2c(c1)OC(C)(C)O2] | Trans-4-[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]cyclohexanecarboxylic acid |
| 8 | 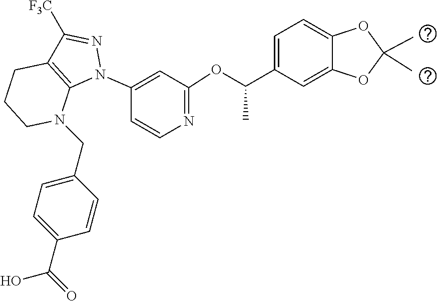 [SMILES: C[C@H](Oc1cc(-n2nc(C(F)(F)F)c3c2N(Cc2ccc(C(=O)O)cc2)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]benzoic acid |
| 9 | 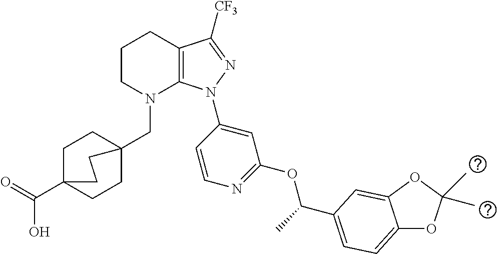 [SMILES: C[C@H](Oc1cc(-n2nc(C(F)(F)F)c3c2N(CC24CCC(C(=O)O)(CC2)CC4)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]bicyclo[2.2.2]octane-1- carboxylic acid |
| 10 | 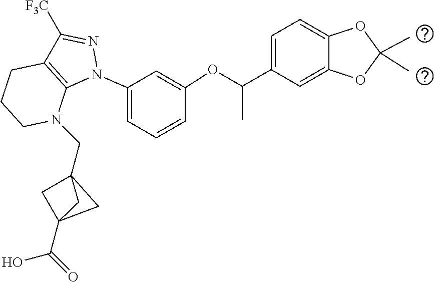 [SMILES: CC(Oc1cccc(-n2nc(C(F)(F)F)c3c2N(CC24CC(C(=O)O)(C2)C4)CCC3)c1)c1ccc2c(c1)OC(C)(C)O2] | 3-[[1-[3-[1-(2,2-difluoro-1,3- benzodioxol-5-yl)ethoxy]phenyl]-3- (trifluoromethyl)-5,6-dihydro-4H- pyrazolo[3,4-b]pyridine-7- yl]methyl]bicyclo[1.1.1]pentane-1- carboxylic acid |
| 11 | 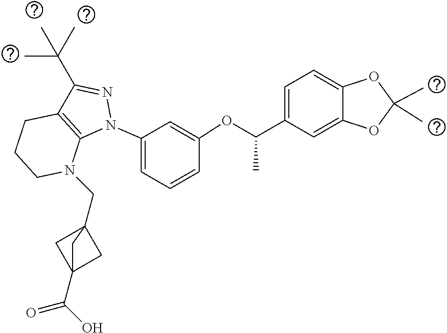 [SMILES: C[C@H](Oc1cccc(-n2nc(C(C)(C)C)c3c2N(CC24CC(C(=O)O)(C2)C4)CCC3)c1)c1ccc2c(c1)OC(C)(C)O2] | 3-[[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- yl]methyl]bicyclo[1.1.1]pentane-1- carboxylic acid |
| 12 | 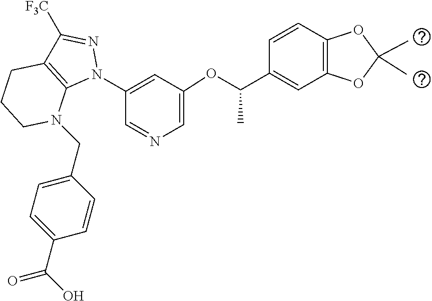 [SMILES: C[C@H](Oc1cncc(-n2nc(C(F)(F)F)c3c2N(Cc2ccc(C(=O)O)cc2)CCC3)c1)c1ccc2c(c1)OC(C)(C)O2] | 4-[[1-[5-[(1S)-1-(2,2-difluoro-1,3- benzodioxol-5-yl)ethoxy]-3-pyridyl]-3- (trifluoromethyl)- 5,6-dihydro-4H-pyrazolo[3,4-b]pyridine- 7-yl]methyl]benzoic acid |
| 13 | 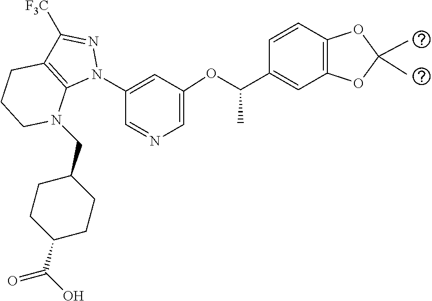 [SMILES: C[C@H](Oc1cncc(-n2nc(C(F)(F)F)c3c2N(C[C@H]2CC[C@H](C(=O)O)CC2)CCC3)c1)c1ccc2c(c1)OC(C)(C)O2] | Trans-4-[[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- yl]methyl]cyclohexanecarboxylic acid |
| 14 | 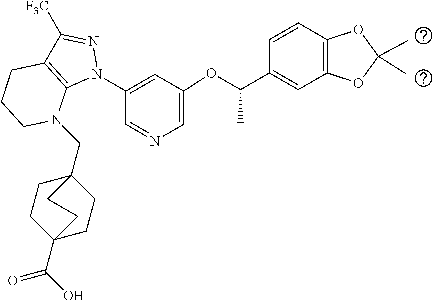 [SMILES: C[C@H](Oc1cncc(-n2nc(C(F)(F)F)c3c2N(CC24CCC(C(=O)O)(CC2)CC4)CCC3)c1)c1ccc2c(c1)OC(C)(C)O2] | 4-[[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- yl]methyl]bicyclo[2.2.2]octane-1- carboxylic acid |
| 15 | 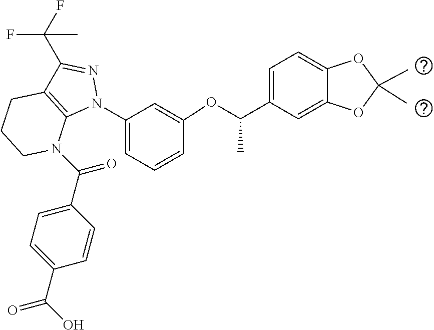 [SMILES: C[C@H](Oc1cccc(-n2nc(C(C)(F)F)c3c2N(C(=O)c2ccc(C(=O)O)cc2)CCC3)c1)c1ccc2c(c1)OC(C)(C)O2] | 4-[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- carbonyl]benzoic acid |
| 16 | 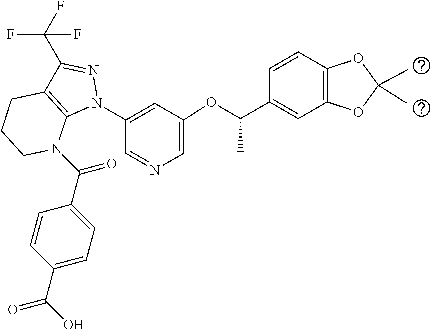 [SMILES: C[C@H](Oc1cncc(-n2nc(C(F)(F)F)c3c2N(C(=O)c2ccc(C(=O)O)cc2)CCC3)c1)c1ccc2c(c1)OC(C)(C)O2] | 4-[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- carbonyl]benzoic acid |
| 17 | 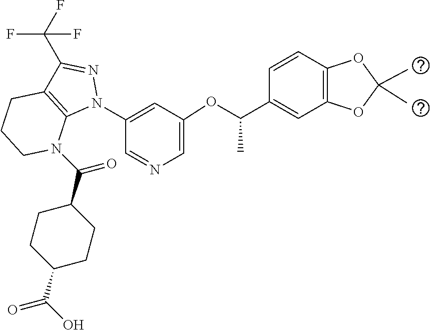 [SMILES: C[C@H](Oc1cncc(-n2nc(C(F)(F)F)c3c2N(C(=O)[C@H]2CC[C@H](C(=O)O)CC2)CCC3)c1)c1ccc2c(c1)OC(C)(C)O2] | Trans-4-[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- carbonyl]cyclohexanecarboxylic acid |
| 18 | 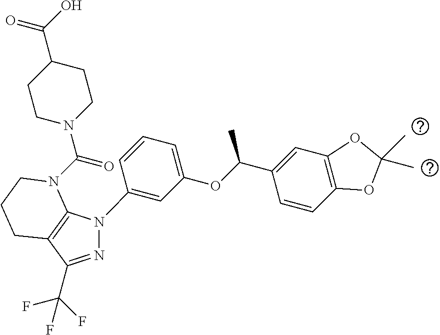 [SMILES: C[C@H](Oc1cccc(-n2nc(C(F)(F)F)c3c2N(C(=O)N2CCC(C(=O)O)CC2)CCC3)c1)c1ccc2c(c1)OC(C)(C)O2] | 1-[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- carbonyl]piperidine-4-carboxylic acid |
| 19 | 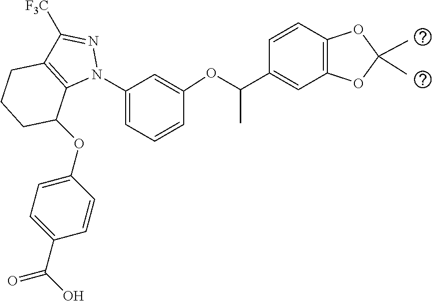 [SMILES: CC(Oc1cccc(-n2nc(C(F)(F)F)c3c2C(Oc2ccc(C(=O)O)cc2)CCC3)c1)c1ccc2c(c1)OC(C)(C)O2] | 4-[[1-[3-[1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 20 | 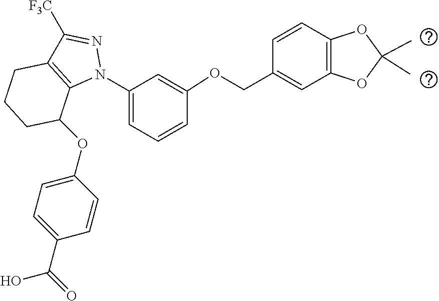 [SMILES: CC1(C)Oc2ccc(COc3cccc(-n4nc(C(F)(F)F)c5c4C(Oc4ccc(C(=O)O)cc4)CCC5)c3)cc2O1] | 4-[[1-[3-[(2,2-difluoro-1,3-benzodioxol- 5-yl)methoxy]phenyl]-3- (trifluoromethyl)-4,5,6,7- tetrahydroindazol-7-yl]oxy]benzoic acid |
| 21 | 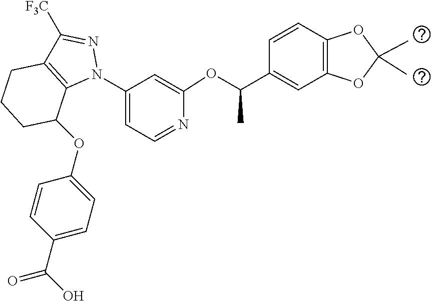 [SMILES: C[C@@H](Oc1cc(-n2nc(C(F)(F)F)c3c2C(Oc2ccc(C(=O)O)cc2)CCC3)ccn1)c1ccc2c(c1)OC(C)(C)O2] | 4-[[1-[2-[(1R)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 22 | 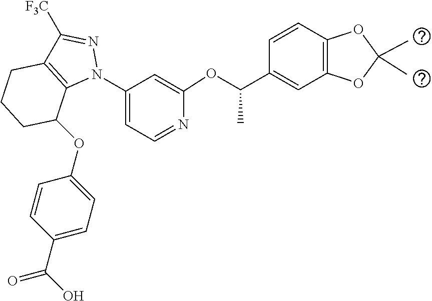 [SMILES: C[C@H](Oc1cc(-n2nc(C(F)(F)F)c3c2C(Oc2ccc(C(=O)O)cc2)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 23 | 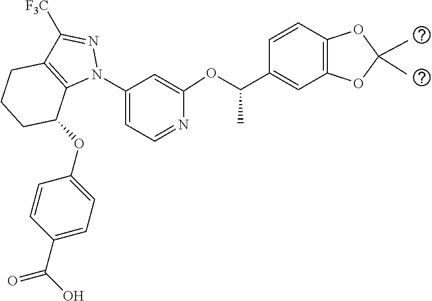 [SMILES: C[C@H](Oc1cc(-n2nc(C(F)(F)F)c3c2[C@H](Oc2ccc(C(=O)O)cc2)CCC3)ccn1)c1ccc2c(c1)OC(C)(C)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 24 | 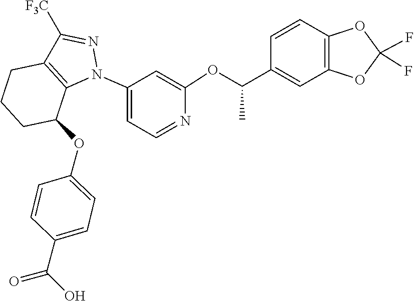 [SMILES: C[C@H](Oc1cc(-n2nc(C(F)(F)F)c3c2[C@@H](Oc2ccc(C(=O)O)cc2)CCC3)ccn1)c1ccc2c(c1)OC(F)(F)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 25 | 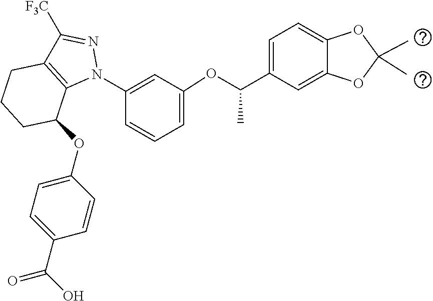 [SMILES: C[C@H](Oc1cccc(-n2nc(C(F)(F)F)c3c2[C@@H](Oc2ccc(C(=O)O)cc2)CCC3)c1)c1ccc2c(c1)OC(C)(C)O2] | 4-[[(7S)-1-[3-[(1S)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 26 | 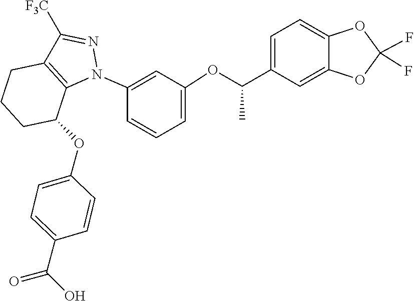 [SMILES: C[C@H](Oc1cccc(-n2nc(C(F)(F)F)c3c2[C@H](Oc2ccc(C(=O)O)cc2)CCC3)c1)c1ccc2c(c1)OC(F)(F)O2] | 4-[[(7R)-1-[3-[(1S)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 27 | 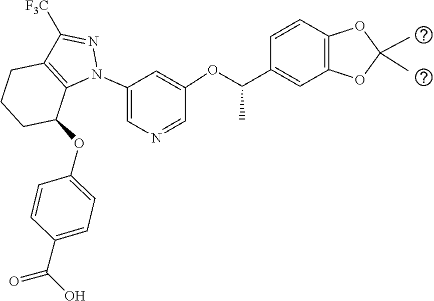 [SMILES: C[C@H](Oc1cncc(-n2nc(C(F)(F)F)c3c2[C@@H](Oc2ccc(C(=O)O)cc2)CCC3)c1)c1ccc2c(c1)OC(C)(C)O2] | 4-[[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 28 | 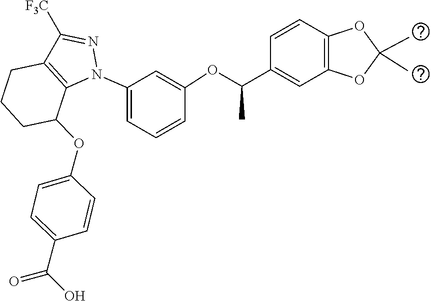 [SMILES: C[C@@H](Oc1cccc(-n2nc(C(F)(F)F)c3c2C(Oc2ccc(C(=O)O)cc2)CCC3)c1)c1ccc2c(c1)OC(C)(C)O2] | 4-[[1-[3-[(1R)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 29 | 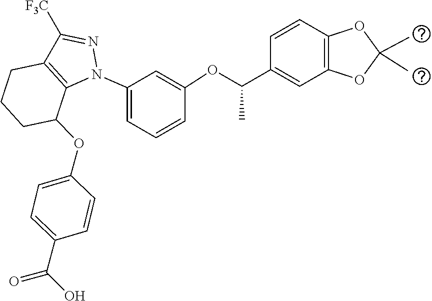 [SMILES: C[C@H](Oc1cccc(-n2nc(C(F)(F)F)c3c2C(Oc2ccc(C(=O)O)cc2)CCC3)c1)c1ccc2c(c1)OC(C)(C)O2] | 4-[1-[3-[(1S)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 30 | 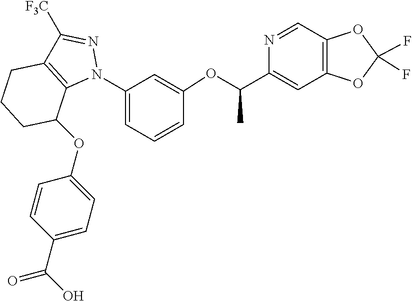 [SMILES: C[C@@H](Oc1cccc(-n2nc(C(F)(F)F)c3c2C(Oc2ccc(C(=O)O)cc2)CCC3)c1)c1cc2c(cn1)OC(F)(F)O2] | 4-[[1-[3-[(1R)-1-(2,2-difluoro- [1,3]dioxolo[4,5-c]pyridine-6- yl)ethoxy]phenyl]-3-(trifluoromethyl)- 4,5,6,7-tetrahydroindazol-7- yl[oxy]benzoic acid |
| 31 | 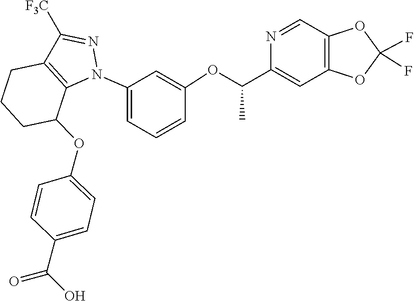 [SMILES: C[C@H](Oc1cccc(-n2nc(C(F)(F)F)c3c2C(Oc2ccc(C(=O)O)cc2)CCC3)c1)c1cc2c(cn1)OC(F)(F)O2] | 4-[[1-[3-[(1S)-1-(2,2-difluoro- [1,3]dioxolo[4,5-c]pyridine-6- yl)ethoxy]phenyl]-3-(trifluoromethyl)- 4,5,6,7-tetrahydroindazol-7- yl[oxy]benzoic acid |
| 32 | 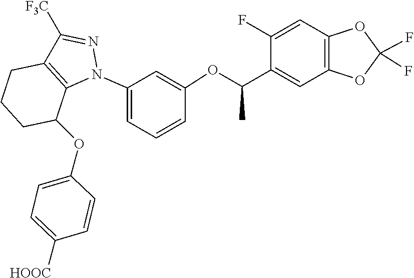 [SMILES: C[C@@H](Oc1cccc(-n2nc(C(F)(F)F)c3c2C(Oc2ccc(C(=O)O)cc2)CCC3)c1)c1cc2c(cc1F)OC(F)(F)O2] | 4-[[1-[3-[(1R)-1-(2,2,6-trifluoro-1,3- benzodioxol-5-yl)ethoxy]phenyl]-3- (trifluoromethyl)-4,5,6,7- tetrahydroindazol-7-yl]oxy]benzoic acid |
| 33 | 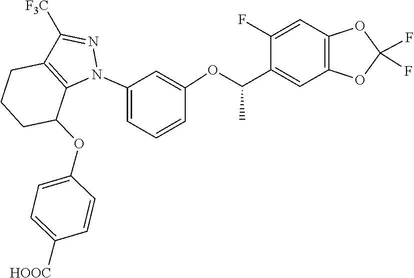 [SMILES: C[C@H](Oc1cccc(-n2nc(C(F)(F)F)c3c2C(Oc2ccc(C(=O)O)cc2)CCC3)c1)c1cc2c(cc1F)OC(F)(F)O2] | 4-[[1-[3-[(1S)-1-(2,2,6-trifluoro-1,3- benzodioxol-5-yl)ethoxy]phenyl]-3- (trifluoromethyl)-4,5,6,7- tetrahydroindazol-7-yl]oxy]benzoic acid |
| 34 | 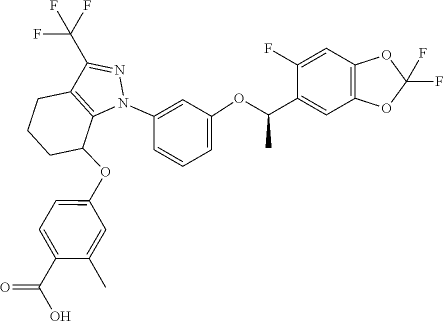 [SMILES: Cc1cc(OC2CCCc3c(C(F)(F)F)nn(-c4cccc(O[C@H](C)c5cc6c(cc5F)OC(F)(F)O6)c4)c32)ccc1C(=O)O] | 2-methyl-4-[[1-[3-[(1R)-1-(2,2,6- trifluoro-1,3-benzodioxol-5- yl)ethoxy]phenyl]-3-(trifluoromethyl)- 4,5,6,7-tetrahydroindazol-7- yl[oxy]benzoic acid |
| 35 | 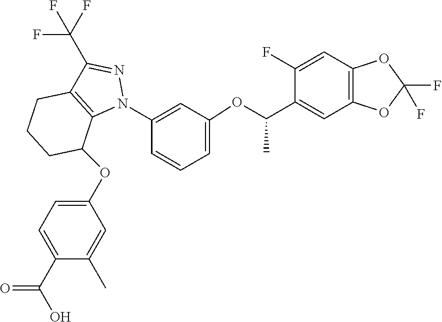 [SMILES: Cc1cc(OC2CCCc3c(C(F)(F)F)nn(-c4cccc(O[C@@H](C)c5cc6c(cc5F)OC(F)(F)O6)c4)c32)ccc1C(=O)O] | 2-methyl-4-[1-[3-[(1S)-1-(2,2,6- trifluoro-1,3-benzodioxol-5- yl)ethoxy]phenyl]-3-(trifluoromethyl)- 4,5,6,7-tetrahydroindazol-7- yl]oxy]benzoic acid |
| 36 | 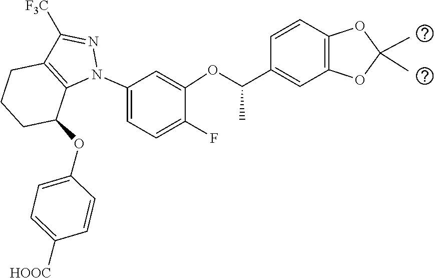 [SMILES: C[C@H](Oc1cc(-n2nc(C(F)(F)F)c3c2[C@@H](Oc2ccc(C(=O)O)cc2)CCC3)ccc1F)c1ccc2c(c1)OC(C)(C)O2] | 4-[[(7S)-1-[3-[(1S)-1-(2,2-difluoro-1,3- benzodioxol-5-yl)ethoxy]-4-fluoro- phenyl]-3-(trifluoromethyl)-4,5,6,7- tetrahydroindazol-7-yl]oxy]benzoic acid |
| 37 | 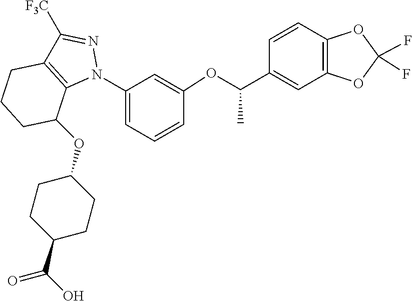 [SMILES: C[C@H](Oc1cccc(-n2nc(C(F)(F)F)c3c2C(O[C@H]2CC[C@H](C(=O)O)CC2)CCC3)c1)c1ccc2c(c1)OC(F)(F)O2] | Trans-4-[1-[3-[(1S)-1-(2,2-difluoro-1,3- benzodioxol-5-yl)ethoxy]phenyl]-3- (trifluoromethyl)-4,5,6,7- tetrahydroindazol-7- yl[oxy]cyclohexanecarboxylic acid |
| 38 | 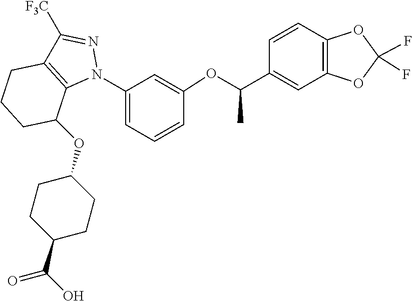 [SMILES: C[C@@H](Oc1cccc(-n2nc(C(F)(F)F)c3c2C(O[C@H]2CC[C@H](C(=O)O)CC2)CCC3)c1)c1ccc2c(c1)OC(F)(F)O2] | Trans-4-[[1-[3-[(1R)-1-(2,2-difluoro-1,3- benzodioxol-5-yl)ethoxyphenyl]-3- (trifluoromethyl)-4,5,6,7- tetrahydroindazol-7- yl[oxy]cyclohexanecarboxylic acid |
| 39 | 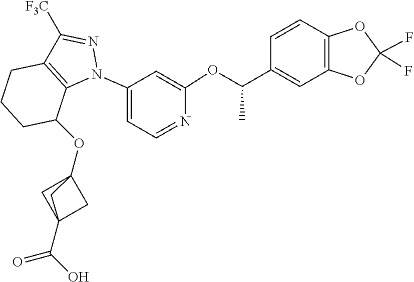 [SMILES: C[C@H](Oc1cc(-n2nc(C(F)(F)F)c3c2C(OC24CC(C(=O)O)(C2)C4)CCC3)ccn1)c1ccc2c(c1)OC(F)(F)O2] | 3-[[1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 40 | 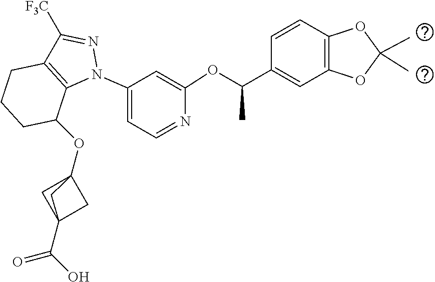 [SMILES: C[C@@H](Oc1cc(-n2nc(C(F)(F)F)c3c2C(OC24CC(C(=O)O)(C2)C4)CCC3)ccn1)c1ccc2c(c1)OC(C)(C)O2] | 3-[1-[2-[(1R)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 41 | 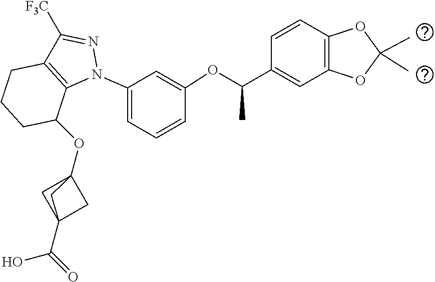 [SMILES: C[C@@H](Oc1cccc(-n2nc(C(F)(F)F)c3c2C(OC24CC(C(=O)O)(C2)C4)CCC3)c1)c1ccc2c(c1)OC(C)(C)O2] | 3-[[1-[3-[(1R)-1-(2,2-difluoro-1,3- benzodioxol-5-yl)ethoxy]phenyl]-3- (trifluoromethyl)-4,5,6,7- tetrahydroindazol-7- yl]oxy]bicyclo[1.1.1]pentane-1- carboxylic acid |
| 42 | 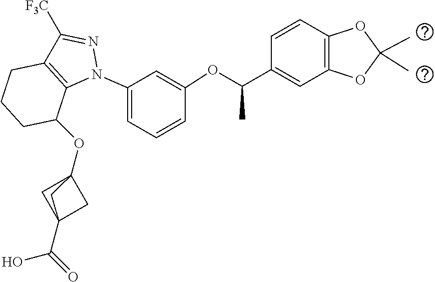 [SMILES: C[C@@H](Oc1cccc(-n2nc(C(F)(F)F)c3c2C(OC24CC(C(=O)O)(C2)C4)CCC3)c1)c1ccc2c(c1)OC(C)(C)O2] | 3-[[1-[3-[(1S)-1-(2,2-difluoro-1,3- benzodioxol-5-yl)ethoxy]phenyl]-3- (trifluoromethyl)-4,5,6,7- tetrahydroindazol-7- yl[oxy]bicyclo[1.1.1]pentane-1- carboxylic acid |
| 43 | 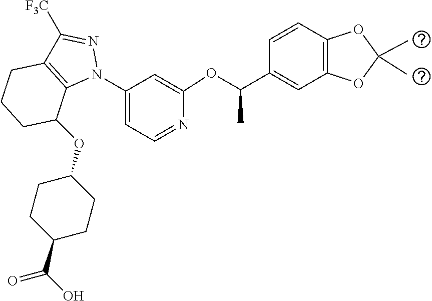 [SMILES: C[C@@H](Oc1cc(-n2nc(C(F)(F)F)c3c2C(O[C@H]2CC[C@H](C(=O)O)CC2)CCC3)ccn1)c1ccc2c(c1)OC(C)(C)O2] | Trans-4-[1-[2-[(1R)-1-(2,2-difluoro-1,3- benzodioxol-5-yl)ethoxy]-4-pyridyl]-3- (trifluoromethyl)-4,5,6,7- tetrahydroindazol-7- yl[oxy]cyclohexanecarboxylic acid |
| 44 | 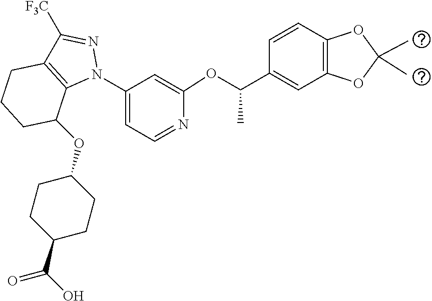 [SMILES: C[C@H](Oc1cc(-n2nc(C(F)(F)F)c3c2C(O[C@H]2CC[C@H](C(=O)O)CC2)CCC3)ccn1)c1ccc2c(c1)OC(C)(C)O2] | Trans-4-[1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl[oxy]cyclohexanecarboxylic acid |
| 45 | 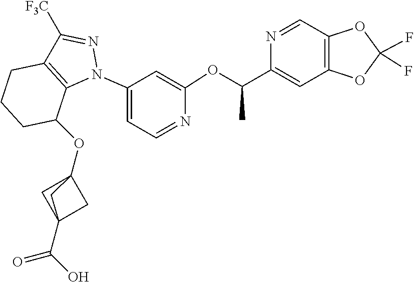 [SMILES: C[C@@H](Oc1cc(-n2nc(C(F)(F)F)c3c2C(OC24CC(C(=O)O)(C2)C4)CCC3)ccn1)c1cc2c(cn1)OC(F)(F)O2] | 3-[[1-[2-[(1S)-1-(2,2-difluoro- [1,3]dioxolo[4,5-c]pyridine-6- yl)ethoxy]-4-pyridyl]-3- (trifluoromethyl)-4,5,6,7- tetrahydroindazol-7- yl]oxy]bicyclo[1.1.1]pentane-1- carboxylic acid |
| 46 | 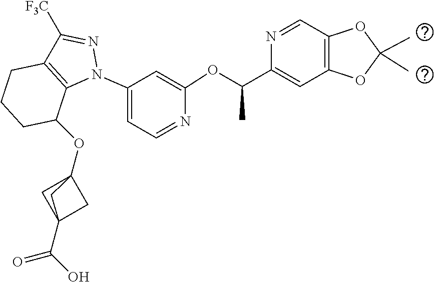 [SMILES: C[C@@H](Oc1cc(-n2nc(C(F)(F)F)c3c2C(OC24CC(C(=O)O)(C2)C4)CCC3)ccn1)c1cc2c(cn1)OC(C)(C)O2] | 3-[[1-[2-[(1R)-1-(2,2-difluoro- [1,3]dioxolo[4,5-c]pyridine-6- yl)ethoxy]-4-pyridyl]-3- (trifluoromethyl)-4,5,6,7- tetrahydroindazol-7- yl]oxy]bicyclo[1.1.1]pentane-1- carboxylic acid |
| 47 | 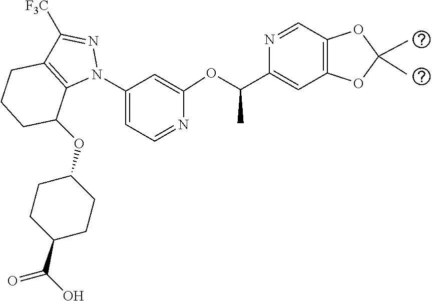 [SMILES: C[C@@H](Oc1cc(-n2nc(C(F)(F)F)c3c2C(O[C@H]2CC[C@H](C(=O)O)CC2)CCC3)ccn1)c1cc2c(cn1)OC(C)(C)O2] | 4-[[1-[2-[(1R)-1-(2,2-difluoro- [1,3]dioxolo[4,5-c]pyridine-6- yl)ethoxy]-4-pyridyl]-3- (trifluoromethyl)-4,5,6,7- tetrahydroindazol-7- yl[oxy]cyclohexanecarboxylic acid |
| 48 | 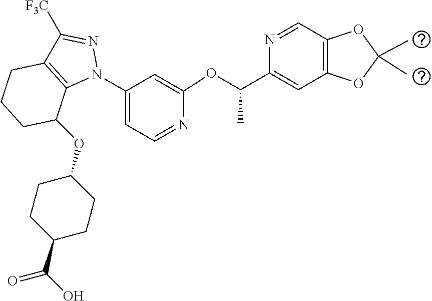 [SMILES: C[C@H](Oc1cc(-n2nc(C(F)(F)F)c3c2C(O[C@H]2CC[C@H](C(=O)O)CC2)CCC3)ccn1)c1cc2c(cn1)OC(C)(C)O2] | Trans-4-[[1-[2-[(1S)-1-(2,2-difluoro- [1,3]dioxolo[4,5-c]pyridine-6- yl)ethoxy]-4-pyridyl]-3- (trifluoromethyl)-4,5,6,7- tetrahydroindazol-7- yl[oxy]cyclohexanecarboxylic acid |
| 49 | 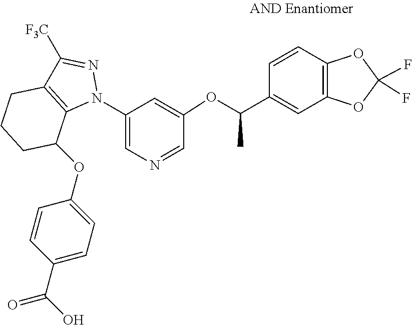 [SMILES: C[C@@H](Oc1cncc(-n2nc(C(F)(F)F)c3c2C(Oc2ccc(C(=O)O)cc2)CCC3)c1)c1ccc2c(c1)OC(F)(F)O2] | 4-[[1-[5-[rac-(1R)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 50 | 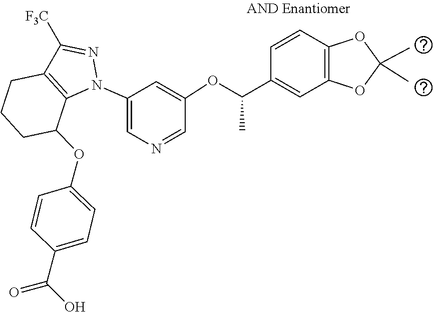 [SMILES: C[C@H](Oc1cncc(-n2nc(C(F)(F)F)c3c2C(Oc2ccc(C(=O)O)cc2)CCC3)c1)c1ccc2c(c1)OC(C)(C)O2] | 4-[[1-[5-[rac-(1S)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 51 | 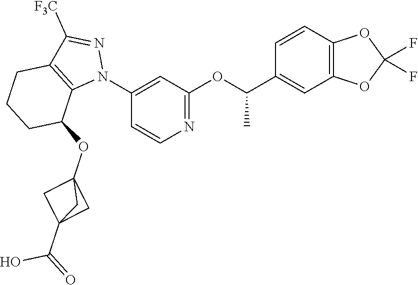 [SMILES: C[C@H](Oc1cc(-n2nc(C(F)(F)F)c3c2[C@@H](OC24CC(C(=O)O)(C2)C4)CCC3)ccn1)c1ccc2c(c1)OC(F)(F)O2] | 3-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl[oxy]bicyclo[1.1.1]pentane-1- carboxylic acid |
| 52 | 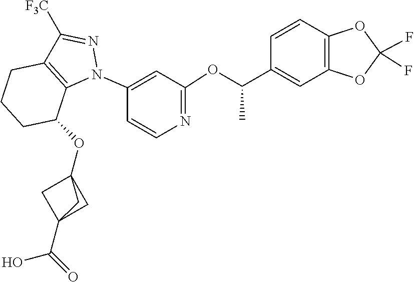 [SMILES: C[C@H](Oc1cc(-n2nc(C(F)(F)F)c3c2[C@H](OC24CC(C(=O)O)(C2)C4)CCC3)ccn1)c1ccc2c(c1)OC(F)(F)O2] | 3-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 53 | 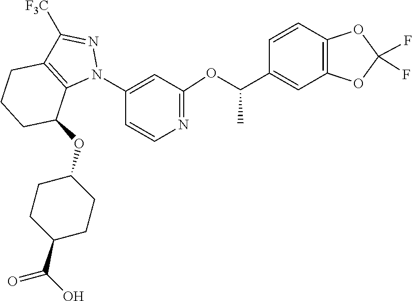 [SMILES: C[C@H](Oc1cc(-n2nc(C(F)(F)F)c3c2[C@@H](O[C@H]2CC[C@H](C(=O)O)CC2)CCC3)ccn1)c1ccc2c(c1)OC(F)(F)O2] | Trans-4-[[(7S)-1-[2-[(1S)-1-(2,2- difluoro-1,3-benzodioxol-5-yl)ethoxy]- 4-pyridyl]-3-(trifluoromethyl)-4,5,6,7- tetrahydroindazol-7- yl[oxy]cyclohexanecarboxylic acid |
| 54 | 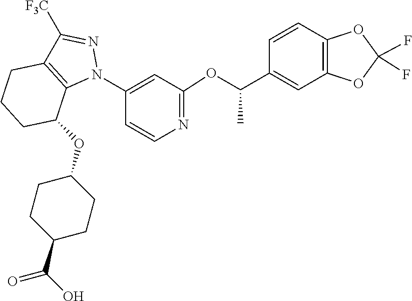 [SMILES: C[C@H](Oc1cc(-n2nc(C(F)(F)F)c3c2[C@H](O[C@H]2CC[C@H](C(=O)O)CC2)CCC3)ccn1)c1ccc2c(c1)OC(F)(F)O2] | Trans-4-[(7R)-1-[2-[(1S)-1-(2,2- difluoro-1,3-benzodioxol-5-yl)ethoxy]- 4-pyridyl]-3-(trifluoromethyl)-4,5,6,7- tetrahydroindazol-7- yl[oxy]cyclohexanecarboxylic acid |
| 55 | 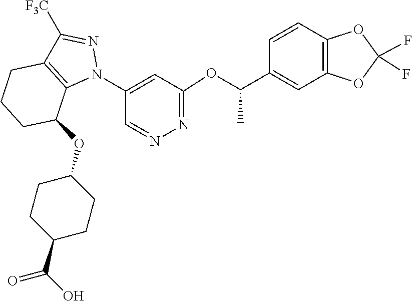 [SMILES: C[C@H](Oc1cc(-n2nc(C(F)(F)F)c3c2[C@@H](O[C@H]2CC[C@H](C(=O)O)CC2)CCC3)cnn1)c1ccc2c(c1)OC(F)(F)O2] | Trans-4-[[(7S)-1-[6-[(1S)-1-(2,2- difluoro-1,3-benzodioxol-5- yl)ethoxy]pyridazin-4-yl]-3- (trifluoromethyl)-4,5,6,7- tetrahydroindazol-7- yl[oxy]cyclohexanecarboxylic acid |
| 56 | 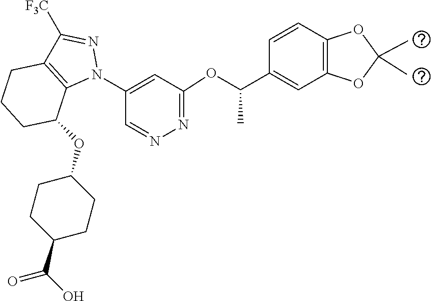 [SMILES: C[C@H](Oc1cc(-n2nc(C(F)(F)F)c3c2[C@H](O[C@H]2CC[C@H](C(=O)O)CC2)CCC3)cnn1)c1ccc2c(c1)OC(C)(C)O2] | Trans-4-[[(7R)-1-[6-[(1S)-1-(2,2- difluoro-1,3-benzodioxol-5- yl)ethoxy]pyridazin-4-yl]-3- (trifluoromethyl)-4,5,6,7- tetrahydroindazol-7- yl[oxy]cyclohexanecarboxylic acid |
| 57 | 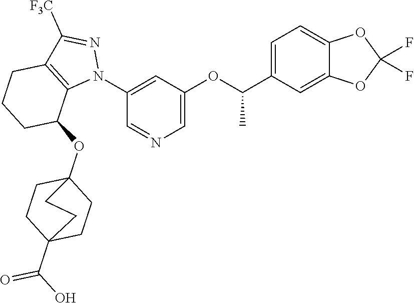 [SMILES: C[C@H](Oc1cncc(-n2nc(C(F)(F)F)c3c2[C@@H](OC24CCC(C(=O)O)(CC2)CC4)CCC3)c1)c1ccc2c(c1)OC(F)(F)O2] | 4-[[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl]oxy]bicyclo[2.2.2]octane-1- carboxylic acid |
| 58 | 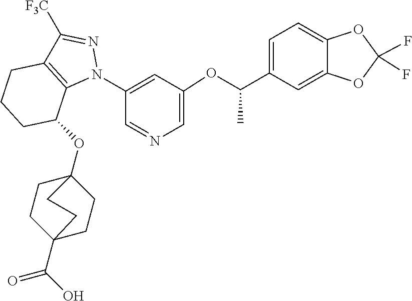 [SMILES: C[C@H](Oc1cncc(-n2nc(C(F)(F)F)c3c2[C@H](OC24CCC(C(=O)O)(CC2)CC4)CCC3)c1)c1ccc2c(c1)OC(F)(F)O2] | 4-[[(7R)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl[oxy]bicyclo[2.2.2]octane-1- carboxylic acid |
| 59 | 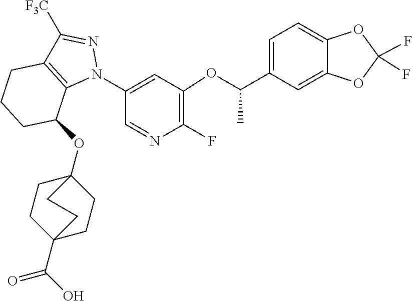 [SMILES: C[C@H](Oc1cc(-n2nc(C(F)(F)F)c3c2[C@@H](OC24CCC(C(=O)O)(CC2)CC4)CCC3)cnc1F)c1ccc2c(c1)OC(F)(F)O2] | 4-[[(7S)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7- yl[oxy]bicyclo[2.2.2]octane-1- carboxylic acid |
| 60 | 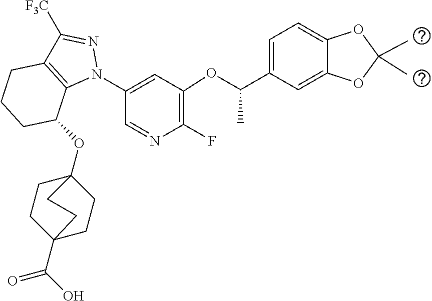 [SMILES: C[C@H](Oc1cc(-n2nc(C(F)(F)F)c3c2[C@H](OC24CCC(C(=O)O)(CC2)CC4)CCC3)cnc1F)c1ccc2c(c1)OC(C)(C)O2] | 4-[[(7R)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7- yl[oxy]bicyclo[2.2.2]octane-1- carboxylic acid |
| 61 | 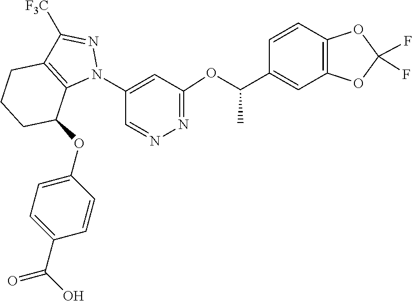 [SMILES: C[C@H](Oc1cc(-n2nc(C(F)(F)F)c3c2[C@@H](Oc2ccc(C(=O)O)cc2)CCC3)cnn1)c1ccc2c(c1)OC(F)(F)O2] | 4-[[(7S)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7-yl]oxy]benzoic acid |
| 62 | 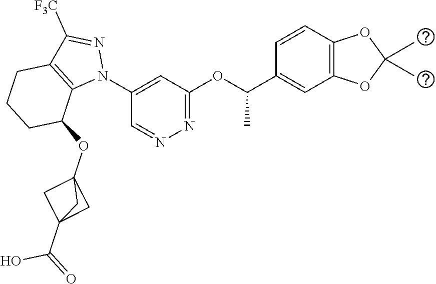 [SMILES: C[C@H](Oc1cc(-n2nc(C(F)(F)F)c3c2[C@@H](OC24CC(C(=O)O)(C2)C4)CCC3)cnn1)c1ccc2c(c1)OC(C)(C)O2] | 3-[[(7S)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7- yl]oxy]bicyclo[1.1.1]pentane-1- carboxylic acid |
| 63 | 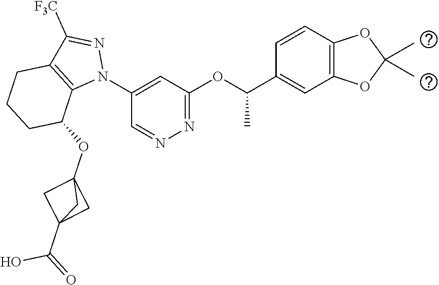 [SMILES: C[C@H](Oc1cc(-n2nc(C(F)(F)F)c3c2[C@H](OC24CC(C(=O)O)(C2)C4)CCC3)cnn1)c1ccc2c(c1)OC(C)(C)O2] | 3-[[(7R)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7- yl]oxy]bicyclo[1.1.1]pentane-1- carboxylic acid |
| 64 | 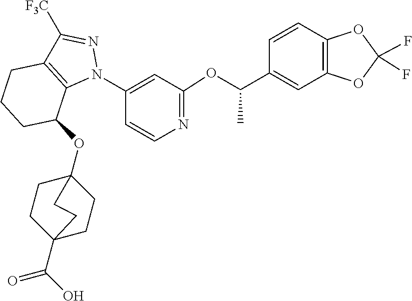 [SMILES: C[C@H](Oc1cc(-n2nc(C(F)(F)F)c3c2[C@@H](OC24CCC(C(=O)O)(CC2)CC4)CCC3)ccn1)c1ccc2c(c1)OC(F)(F)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[2.2.2]octane-1- carboxylic acid |
| 65 | 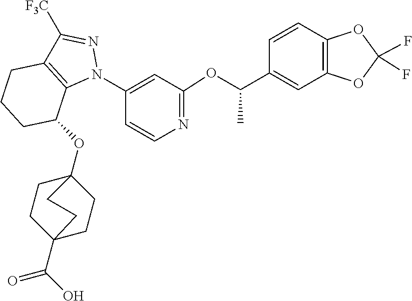 [SMILES: C[C@H](Oc1cc(-n2nc(C(F)(F)F)c3c2[C@H](OC24CCC(C(=O)O)(CC2)CC4)CCC3)ccn1)c1ccc2c(c1)OC(F)(F)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl[oxy]bicyclo[2.2.2]octane-1- carboxylic acid |
| 66 | 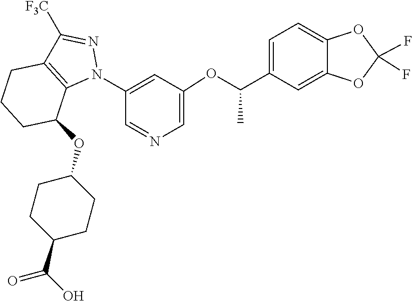 [SMILES: C[C@H](Oc1cncc(-n2nc(C(F)(F)F)c3c2[C@@H](O[C@H]2CC[C@H](C(=O)O)CC2)CCC3)c1)c1ccc2c(c1)OC(F)(F)O2] | Trans-4-[[(7S)-1-[5-[(1S)-1-(2,2- difluoro-1,3-benzodioxol-5-yl)ethoxy]- 3-pyridyl]-3-(trifluoromethyl)-4,5,6,7- tetrahydroindazol-7- yl[oxy]cyclohexanecarboxylic acid |
| 67 | 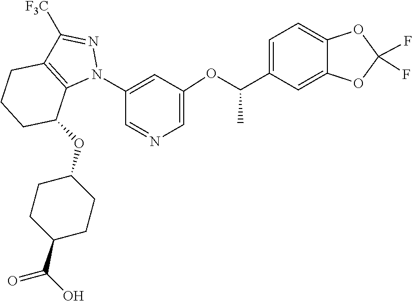 [SMILES: C[C@H](Oc1cncc(-n2nc(C(F)(F)F)c3c2[C@H](O[C@H]2CC[C@H](C(=O)O)CC2)CCC3)c1)c1ccc2c(c1)OC(F)(F)O2] | Trans-4-[[(7R)-1-[5-[(1S)-1-(2,2- difluoro-1,3-benzodioxol-5-yl)ethoxy]- 3-pyridyl]-3-(trifluoromethyl)-4,5,6,7- tetrahydroindazol-7- yl[oxy]cyclohexanecarboxylic acid |
| 68 | 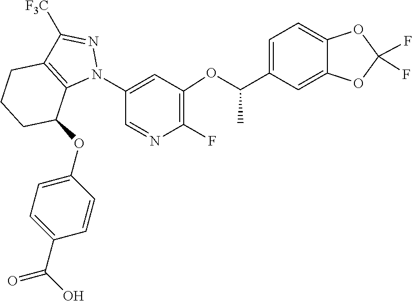 [SMILES: C[C@H](Oc1cc(-n2nc(C(F)(F)F)c3c2[C@@H](Oc2ccc(C(=O)O)cc2)CCC3)cnc1F)c1ccc2c(c1)OC(F)(F)O2] | 4-[[(7S)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7-yl]oxy]benzoic acid |
| 69 | 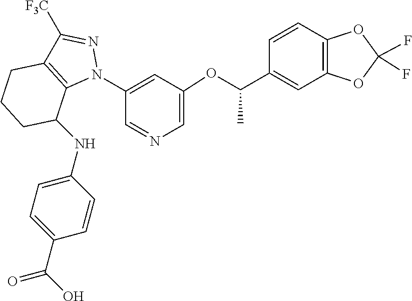 [SMILES: C[C@H](Oc1cncc(-n2nc(C(F)(F)F)c3c2C(Nc2ccc(C(=O)O)cc2)CCC3)c1)c1ccc2c(c1)OC(F)(F)O2] | 4-[[1-[5-[(1S)-1-(2,2-difluoro-1,3- benzodioxol-5-yl)ethoxy]-3-pyridyl]-3- (trifluoromethyl)-4,5,6,7- tetrahydroindazol-7-yl]amino]benzoic acid |
| 70 | 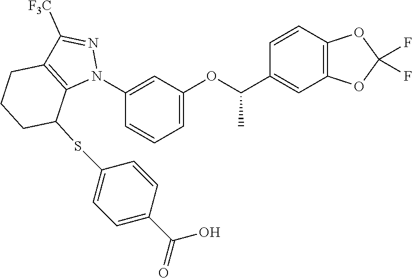 [SMILES: C[C@H](Oc1cccc(-n2nc(C(F)(F)F)c3c2C(Sc2ccc(C(=O)O)cc2)CCC3)c1)c1ccc2c(c1)OC(F)(F)O2] | 4-[1-[3-[(1S)-1-(2,2-difluoro-1,3- benzodioxol-5-yl)ethoxy]phenyl]-3- (trifluoromethyl)-4,5,6,7- tetrahydroindazol-7-yl]sulfanyl]benzoic acid |
| 71 | 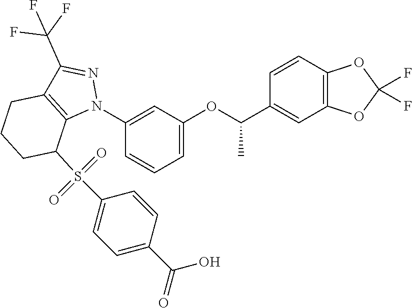 [SMILES: C[C@H](Oc1cccc(-n2nc(C(F)(F)F)c3c2C(S(=O)(=O)c2ccc(C(=O)O)cc2)CCC3)c1)c1ccc2c(c1)OC(F)(F)O2] | 4-[[1-[3-[(1S)-1-(2,2-difluoro-1,3- benzodioxol-5-yl)ethoxy]phenyl]-3- (trifluoromethyl)-4,5,6,7- tetrahydroindazol-7-yl]sulfonyl]benzoic acid |
| 72 | 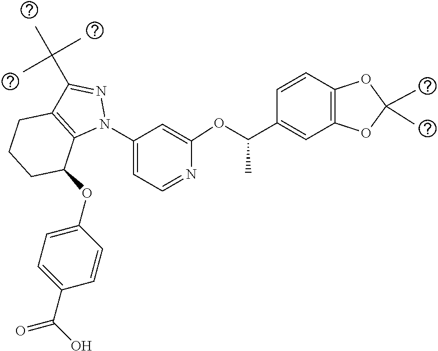 [SMILES: C[C@H](Oc1cc(-n2nc(C(C)(C)C)c3c2[C@@H](Oc2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]sulfanyl]benzoic acid |
| 73 | 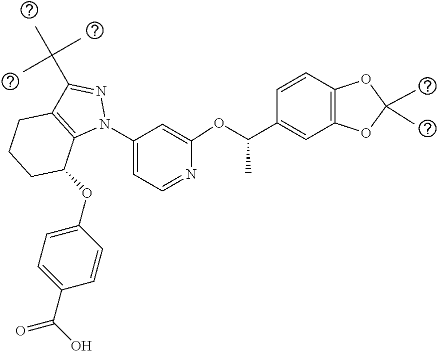 [SMILES: C[C@H](Oc1cc(-n2nc(C(C)(C)C)c3c2[C@H](Oc2ccc(C(=O)O)cc2)CCC3)ccn1)c1ccc2c(c1)OC(C)(C)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]sulfanyl]benzoic acid |

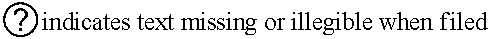

or a pharmaceutically acceptable salt thereof.
24. A pharmaceutical composition comprising a compound of any of embodiments 1-23 and a pharmaceutically acceptable adjuvant.
25. A method of treating a CFTR-mediated disease or disorder comprising administering a patient in need there of a compound any of embodiments 1-23 or a pharmaceutical composition of embodiment 24.
26. The method of embodiment 25, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.
27. The method of embodiment 25 or 26, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.
28. The method of any one of embodiments 25-27, wherein the disease or condition is cystic fibrosis.
29. A method of treating cystic fibrosis in a subject, comprising administering to the subject a therapeutically effective amount of a compound of any one of embodiments 1-23 or a pharmaceutical composition of embodiment 24.
30. The method of embodiment 29, wherein the subject is human.

### Exemplification

### General Procedures

### Preparation of Intermediates

Scheme A-D illustrate the methods for preparation of Intermediates A-D generally used for the synthesis of the compound of formula (I) and/or (II) from commercially available starting materials or readily prepared by the known reactions familiar to one skilled in the art.

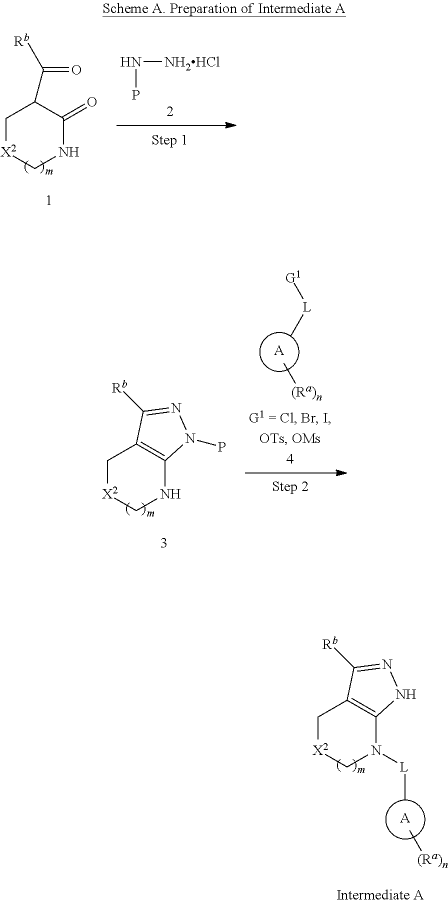

Scheme A illustrates the synthetic method for preparation of Intermediate A. As shown in Scheme A, commercially available or easily prepared starting material 1 is condensed with hydrazine 2, bearing an appropriate protecting group P, such as 4-methoxybenzyl, using a dehydrating reagent, such as 2,4-bis(4-methoxyphenyl)-1,3-dithia-2,4-diphosphetane-2,4-disulfide, with heating (Step 1) to provide Compound 3. Compound 3 can be alkylated at its piperidine nitrogen with alkylating agent 4 (Step 2) with removal of the protecting group 4-methoxybenzyl under the reaction conditions to obtain Intermediate A.

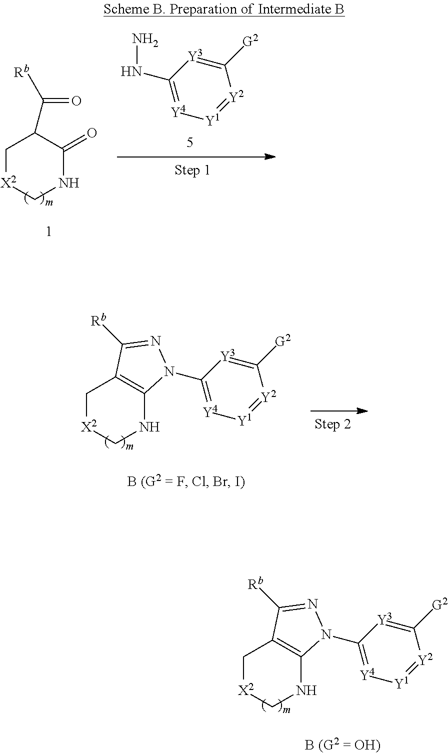

Similar to Scheme A, commercially available or easily prepared starting material 1 is condensed with aryl hydrazine 5 bearing propriate groups as required in Formula (I) under heating conditions to obtain Intermediate B, G²=F, Cl, Br, and I. This intermediate can be further converted (Step 2) into hydroxyl group (Intermediate B, G²=OH) catalyzed by palladium mediated reagents. For example, bromide can be converted to hydroxyl by using cesium hydroxide monohydrate catalyzed by Pd catalyst, such as tris(dibenzylideneacetone) dipalladium.

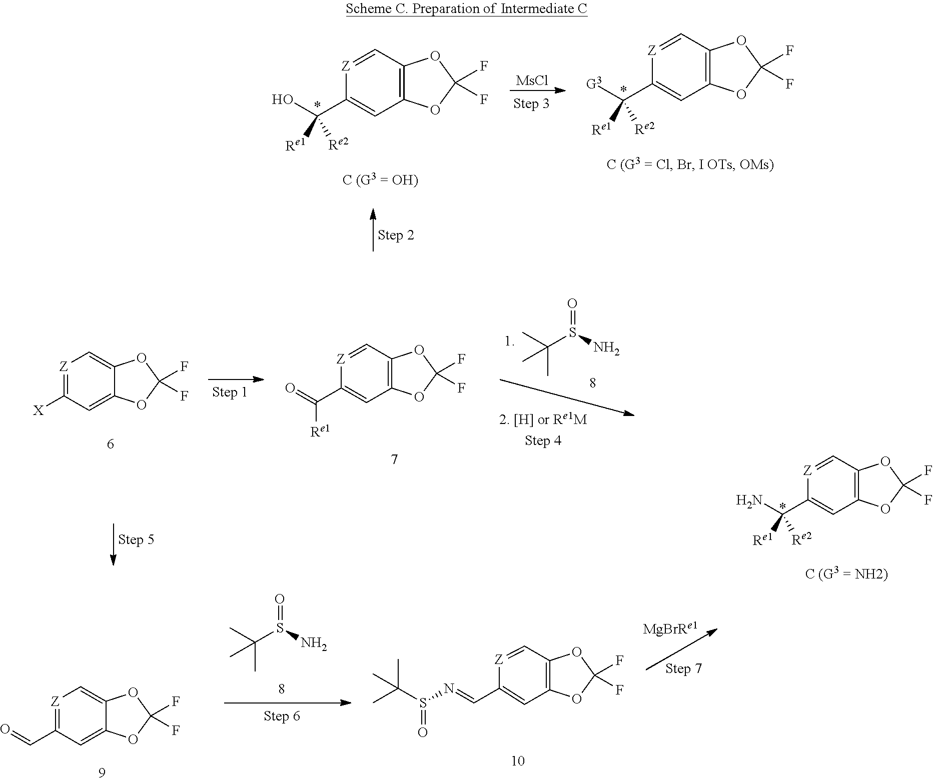

Scheme C illustrates the preparation of Intermediate C (G³=OH, NH₂, Cl, Br, I, OTs, OMs). The aryl halide 6 (X=Cl, Br, I) is converted into the alkyl aryl ketone (Step 1) via available methods known to one skilled in the art, such as coupling with 1-ethoxyvinyltri-n-butyltin or treating halide 6 with nBuLi followed by addition of the acyl halides to obtain the ketone 7. Compound 7 can be reduced (Step 2) with reducing agent, such as sodium borohydride to obtain achiral Intermediate C. Ketone 7 can also be reduced via an enantioselective protocol, such as reducing with BH₃·Me₂S, catalyzed by (S)-2-methyl-CBSoxazaborolidine in good yield and high ee, to obtain enantiomerically pure alcohol Intermediate C (G³=OH). This intermediate can be easily converted (Step 3) into chloride or bromide.

Ketone 7 can be condensed with chiral auxiliary 8 (Step 4), and then subjected to reduction or carbon anion addition conditions to yield Intermediate C (G³=NH₂). Alternatively, halide 6 is converted to aryl aldehyde 9 (Step 5). Compound 9 is condensed with chiral auxiliary 8 (Step 6) catalyzed by titanium (IV) isopropoxide to obtain Compound 10. Grignard or similar metallic agent addition to compound 10 (Step 7) yields Intermediate C (G³=NH₂).

Intermediate D can be synthesized via the following methods (Scheme Da to Dc). The selection of the methods is dictated by the nature and the position of Y (Y¹-Y⁴).

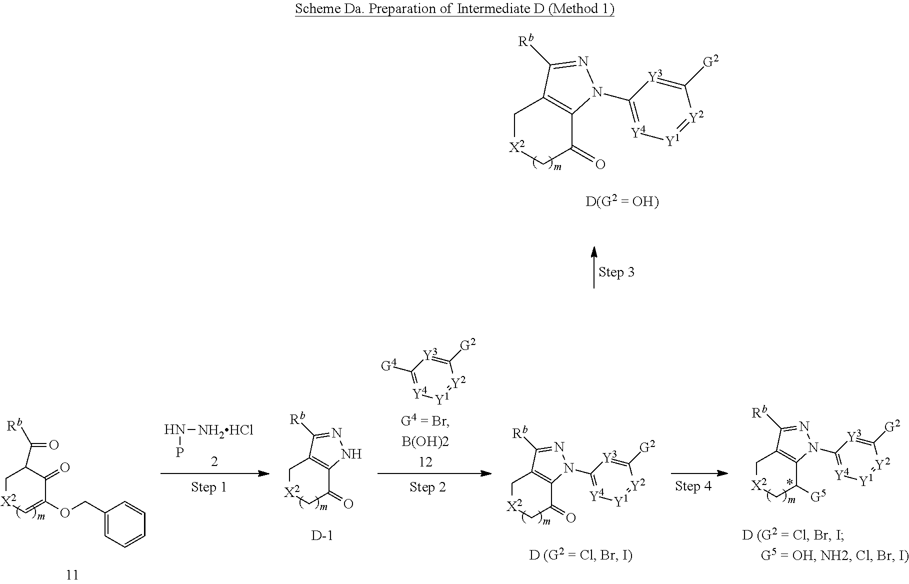

As illustrated in Scheme Da, the ene-1,3-dione 11 is condensed (step 1) with properly protected hydrazine 2 to yield Intermediate D-1. This intermediate (D-1) is coupled with properly substituted aryl analog 12 (Step 2) to give rise to Intermediate D (G²=Cl, Br, I, OH, NH₂). The selection of the reaction conditions is dictated by the nature of functional groups G⁴ and Y²/Y³. For example, if 12 is 3,5-dichloropyridazine, the direct halogen replacement between Intermediate D-1 and 12 (G⁴-Br) can be carried out by using catalyst Cs₂CO₃ with heating. Intermediate D (G²=Cl, Br, I) can be further transformed into other Intermediate D (G²=OH) (Step 3) and D (G²=Cl, Br, I; G⁴=OH, NH₂, Cl, Br, I) (Step 4) to meet the coupling needs for the next step of the reactions (see Scheme 1-6).

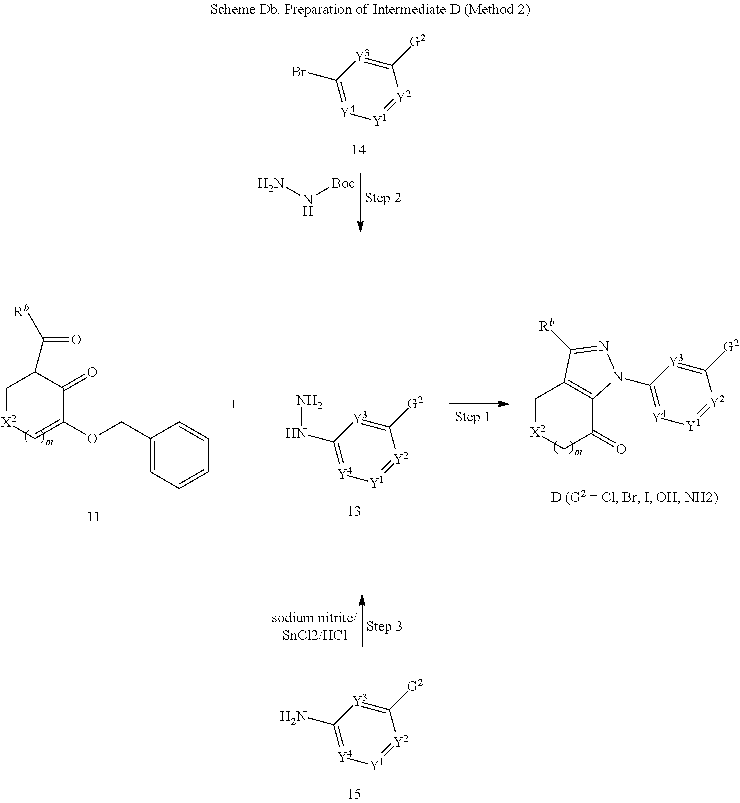

Scheme Db illustrates an alternative method to prepare Intermediate D. The ene-1,3-dione 11 is condensed (Step 1) with aryl hydrazine 13 bearing desired functional group (G²) to form Intermediate D (G2=Cl, Br, I, OH, NH₂) directly. If aryl hydrazine 13 is not commercially available, it can be prepared readily via halogen replacement reactions of 14 with protected hydrazine (Step 2) or amination reaction by treating the aryl amine 15 with sodium nitrite/SnCl₂/HCl (Step 3).

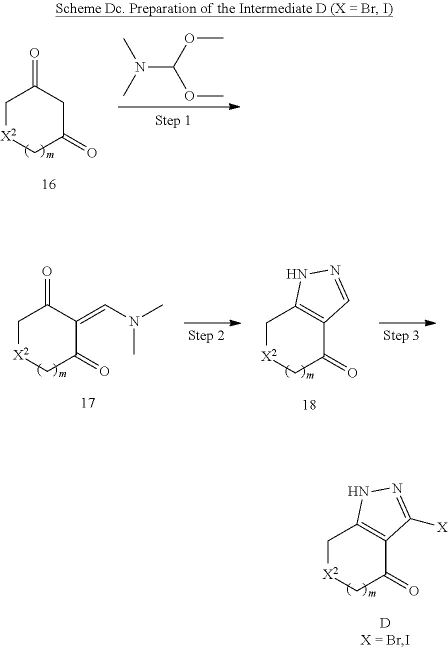

Scheme Dc illustrates another method to prepare Intermediate D (X=Br, I). 1,3-Dione 16 is condensed with DMF-DMA (Step 1) to form Compound 17. This compound is condensed with hydrazine hydrate and cyclized thereafter (Step 2) to yield Compound 18. Halogenation of 18 (Step 3) with NBS or NIS generates desired Intermediate D (X=Br, I).

### Representative Schemes for the Synthesis of Exemplary Compounds of the Present Disclosure

The compounds of Formula (I) and/or (II) of the better disclosure can be better understood in connection with the following synthetic schemes (Scheme 1-6) and methods which illustrate means by which the compounds may be synthesized. The starting materials for the reaction schemes described herein are prepared according to methods described in Preparation Intermediates (Scheme A-D) or commercially available material in some instances. These schemes are Representative schemes with no intention of including all the methods employed for the synthesis of exemplary compounds of the present disclosure. Synthesis of some compounds may lie outside of the scopes in Scheme 1-6. In this case, please refer to the Examples.

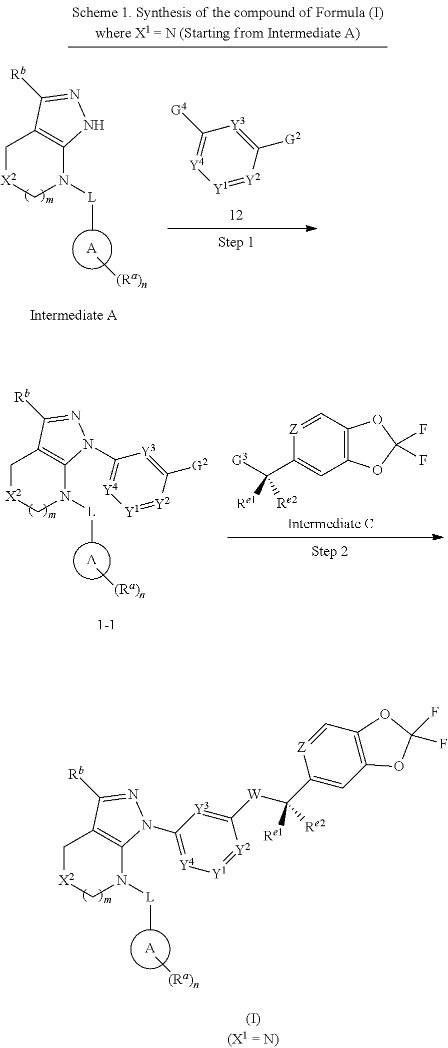

As shown in Scheme 1, Intermediate A can be condensed with substituted aryl 12 under the proper reaction conditions (Step 1) to obtain Compound 1-1. This compound can be further coupled (Step 2) with Intermediate C to obtain, after removal of the carboxylic acid protecting group, such as methyl or ethyl, the compound of Formula (I) where X¹=N. The choice of reagents and reaction conditions in Step 2 is dictated by the nature of the functional groups G², G³ and Y²/Y³). For example, if G² and G³ are both hydroxyl groups, then Mitsunobu reaction conditions are applicable. In this case, the chirality of the carbon atom bearing the R^{e1} and R^{e2} substituents will be inverted. If G² is halogen, G³ should be hydroxyl or amino group, and the appropriate substitution reaction conditions should be chosen. In this case, the chirality of the carbon atom bearing the R^{e1} and R^{e2} substituents is maintained. Similarly, If G² is hydroxyl, then G³ should be a proper leaving group, such as halogen, OTs, or OMs to ensure that the substitution reaction occurs.

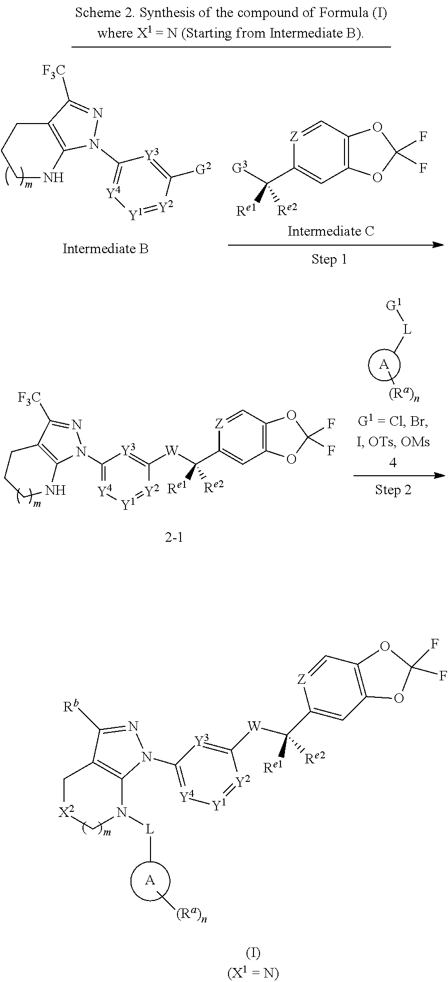

Scheme 2 illustrates an alternative synthetic sequence to that described in Scheme 1 to synthesize the compound of Formula (I) where X¹=N. In Scheme 2, Intermediate B is coupled with Intermediate C (Step 1) to obtain Compound 2-1. The choice of reaction conditions is again dictated by the functional groups involved in the reaction known to one skilled in the art in a manner similar to that of Step 2 in Scheme 1 to obtain Compound 2-1. Compound 2-1 is then coupled with reagent 4 (Step 2, Scheme 2) to obtain the compound of Formula (I) where X¹=N.

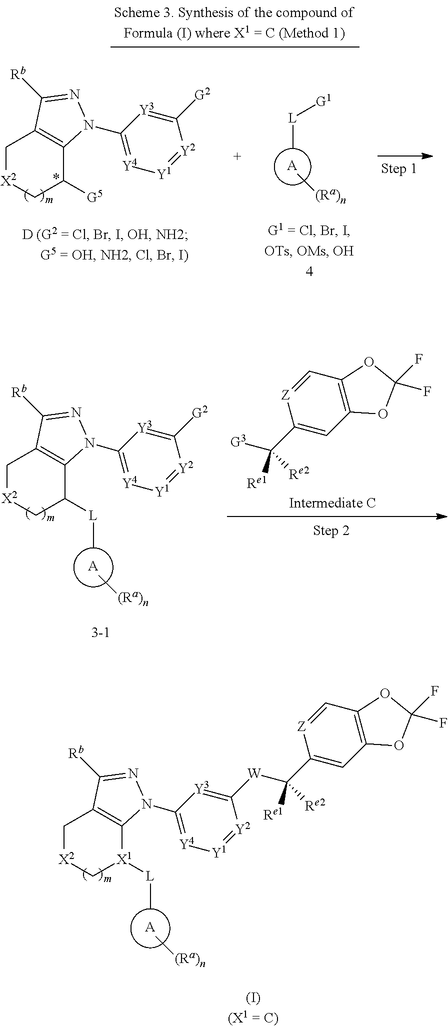

As illustrated in Scheme 3, Intermediate D (G²=Cl, Br, I, OH, NH₂; G⁵=OH, NH₂, Cl, Br, I) is coupled with compound 4 (Step 1) to yield Compound 3-1. The selection of the reaction conditions/reagents is dictated by the functional groups G¹-G³. For example, if G³ is OH or NH₂, then G¹ should be a leaving group to ensure the substitution reaction is carried out. If G⁵ is Cl, Br, I, then G¹ should be S, OH, or NH₂ to allow the substitution reaction to occur. Compound 3-1 is condensed with Intermediate C (Step 2) in similar conditions to Scheme 1, Step 2 to obtain the compound of formula (I) where X¹=carbon.

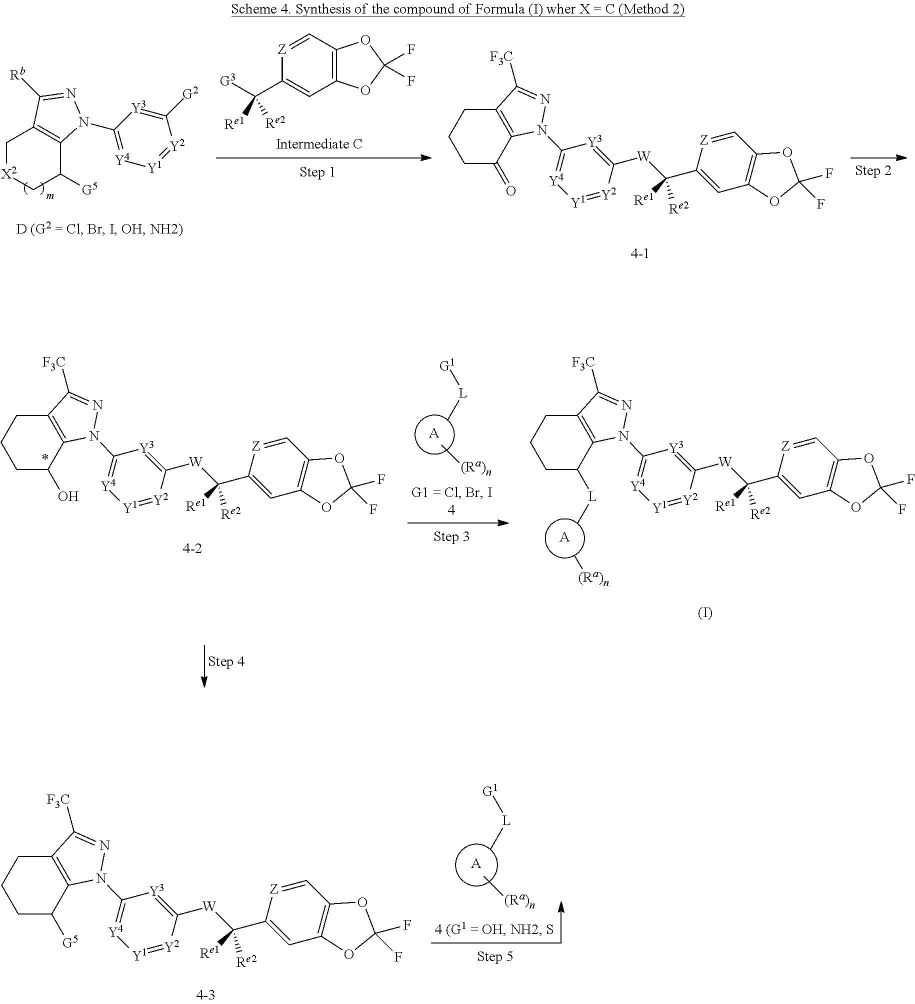

Scheme 4 illustrates an alternative synthetic sequence to that described in Scheme 3 for synthesizing the compound of Formula (I) where X¹=carbon. In Scheme 4, Intermediate D (G²=Cl, Br, I, OH, NH₂) is coupled with Intermediate C (Step 1) under substitution reaction conditions (Step 1) to give Compound 4-1. Reduction of Compound 4-1 (Step 2) can be carried out under achiral conditions using the reducing agent, such as sodium borohydride, or under enantio-selective conditions by using chiral catalysis, such as (R, R)-TsDPEN-RuCl (p-cymene), to afford alcohol 4-2 (* denotes enantiomerically pure analog). Compound 4-2 is then coupled with reagent 4 (Step 3) to obtain the compound of Formula (I) where X¹=carbon. Alternatively, the alcohol 4-2 can be converted (Step 4) into halide or adequate leaving group (such as OTs or OMs) 4-3 (G⁵=Cl, Br, I, OTs, OMs) which then reacts with reagent 4 (G¹=OH, NH₂) (Step 5) to give the compound of Formula (I) where X¹=carbon.

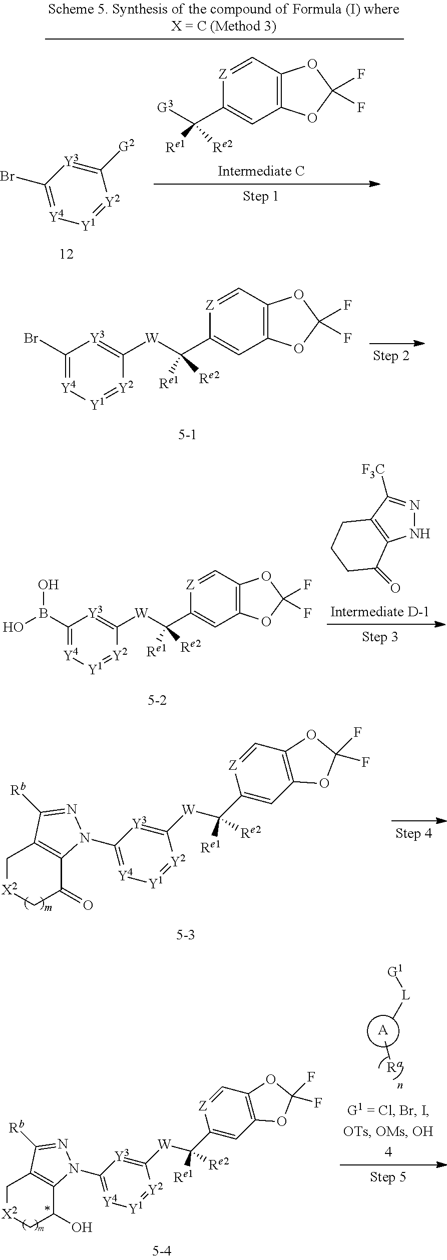

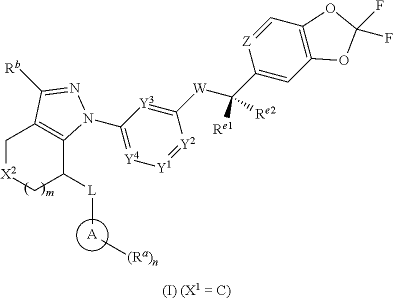

In some other cases, an alternative synthetic sequence is employed as shown in Scheme 5 in order to successfully synthesize the compound of Formula (I). In Scheme 5, Compound 12 is condensed with enantiomerically pure alcohol or amine Intermediate C (Step 1) under Mitsunobu reaction conditions (G²=OH and G³=OH) with the inversion of the stereochemistry to provide Compound 5-1 (W=O). If G³ is an amino group, G² should be a halide. In this case, Pd mediated Buchwald coupling reaction is employed with the retention of the stereochemistry to yield Compound 5-1 (W=O or NH). The bromide 5-1 is converted (Step 2) into a boronic acid or boronic ester (5-2) which is then coupled with Intermediate D-1 (Step 3) to yield Compound 5-3 under Buchwald coupling reaction conditions. Compound 5-3 can be reduced (Step 4) via the conditions described in Step 2 of Scheme 3 to produce alcohol 5-4. The side chain is then installed onto Compound 5-4 (Step 5) using reagent 4 and applying the reaction conditions illustrated in Step 3 of Scheme 3 to provide the compound of Formula (I) following the methods described in Scheme 4, Step 3 or Step 4.

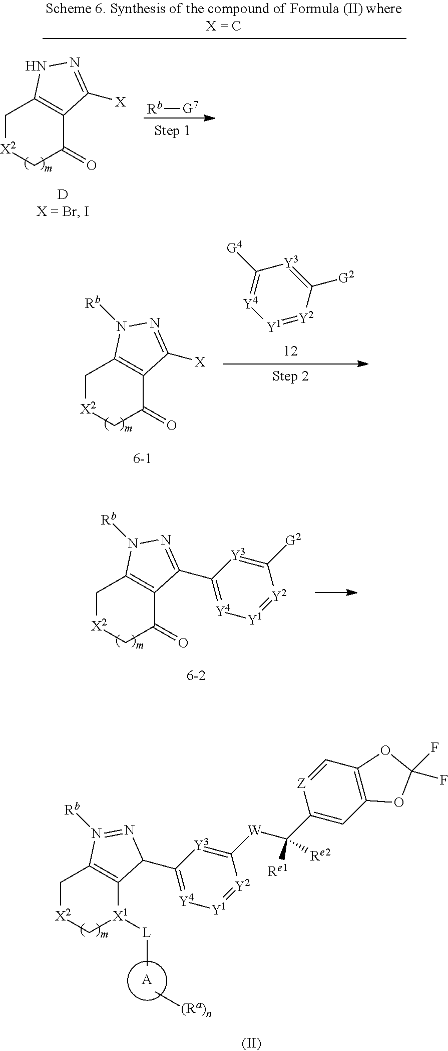

Scheme 6 illustrates the synthesis of compound of Formula (II) where X=C. In Step 1, pyrazole nitrogen (Intermediate D, X=Br, I) is alkylated catalyzed by a strong base, such as sodium hydride, to yield Compound 6-1. This compound can be coupled with 12 (G⁴=B(OH)₂) (Step 2) under standard Suzuki coupling conditions known to one skilled in the art to yield Compound 6-2. The rest of the steps and the strategies (reaction sequences) are similar to those in Schemes 1 to 5.

The enantiomerically pure examples and synthetic intermediates in this present disclosure can be achieved either via chiral separation, such as chiral HPLC or SFC, or through enantioselective reactions.

### Analytical Procedures

High Pressure Liquid Chromatography-Mass Spectrometry (LC-MS) to determine compound retention times (RT) and associated mass ions were performed using one of the following methods.

LC-MS Method 1: Mobile Phase: A: water (0.01% TFA). B: ACN (0.01% TFA).

Gradient: 5%-95% B in 1.5 min. Flow Rate: 2.0 mL/min. Column: Sunfire C18, 4.6×50 mm, 3.5 μm. Oven Temperature: 50° C. Mass Range: 110-1000. UV (214 nm, 254 nm). LC-MS Method 2: Column: Xbridge C18 (2) (4.6×50 mm, 3.5 μm). Mobile phase: H₂O (10 mmol NH₄HCO₃) (A)/ACN (B). Elution program: Gradient from 10 to 95% of B in 1.5 min at 1.8 mL/min. Temperature: 50° C. Detection: UV (214 nm, 254 nm) and MS (ESI, Positive mode, 103 to 800 amu).

LC-MS Method 3: Mobile Phase: A: water (0.01% TFA) B: ACN (0.01% TFA).

Gradient: 5%-95% B in 1.5 min. Flow Rate: 2.0 mL/min. Column: Sunfire C18, 3.5 μm 4.6×50 mm. Oven Temperature: 50° C. Mass Range: 110-1000 UV: 214 nm; 254 nm. LC-MS Method 4: Column: Xbridge C18 (2) (4.6×50 mm, 3.5 μm). Mobile phase: H₂O (10 mmol NH₄HCO₃) (A)/ACN (B). Elution program: Gradient from 5 to 90% of B in 1.4 min at 1.8 mL/min. Temperature: 50° C. Detection: UV (214 nm, 254 nm) and MS (ESI, Positive mode, 105 to 900 amu).

The 1H NMR spectra were collected at 400 MHz on a Gemini 400 or Varian Mercury 400 spectrometer (unless noted otherwise) with an ASW 5 mm probe, and usually recorded at ambient temperature in a deuterated solvent, such as D₂O, DMSO-D₆, CH₃OH-d₄ or CDCl₃ unless otherwise noted. Chemical shift values (8) are indicated in parts per million (ppm) with reference to tetramethylsilane (TMS) as the internal standard.

### Abbreviations

ACN: acetonitrile
Boc: tert-butyloxycarbonyl
DEA: diethyl amine
DBU: 1,8-Diazabicyclo[5.4.0]undec-7-ene
DCE: 1,2-dichloroethane
DCM: dichloromethane
DIAD: diisopropyl azodicarboxylate
DMAP: 4-dimethylaminopyridine
DMSO: dimethyl sulfoxide
dppf: 1,1′-Bis(diphenylphosphino) ferrocene
EA: ethyl acetate
ee: enantiomeric excess
ESI: electron spray ionization
HPLC: high performance liquid chromatography
LC-MS: liquid chromatography-mass spectrometry
MsCl: methanesulfonyl chloride
Pd/C: Palladium on carbon
rt: room temperature
PE: petroleum ether
SFC: supercritical fluid chromatography
TBS: tert-butyldimethylsilyl
TIPS: triisopropylsilyl
THF: tetrahydrofuran
THP: tetrahydropyran
Ts: tosyl
   General Disclaimer about Stereochemistry

It is understood that absolute stereochemistries for all intermediates and examples described herein have not been determined. The assignments of the chiral center(s) to R or S are completely arbitrary and are solely for the purpose of differentiating the different fractions (P1 and P2) eluted out from either flash column chromatography or prep-HPLC, or chiral HPLC, or SFC. There is no association of P1 or P2 with the S or R designations. Methods of determining the absolutely stereochemistry of an enantioenriched sample are known to persons of ordinary skill in the art.

Both ChemDraw and Mol2Nam from OpenEye Scientific software have been used to generate compounds' names. If not consistent, the structure should govern.

### PREPARATION OF INTERMEDIATES

### Preparation of Intermediate A-1

### Methyl 3-((3-(trifluoromethyl)-1,4,5,6-tetrahydro-7H-pyrazolo[3,4-b]pyridine-7-yl)methyl)bicyclo[1.1.1]pentane-1-carboxylate

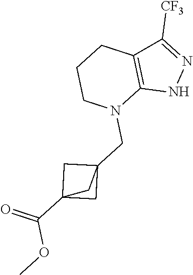 [SMILES: COC(=O)C12CC(CN3CCCc4c(C(F)(F)F)n[nH]c43)(C1)C2]

### Step 1. Synthesis of 1-(4-methoxybenzyl)-3-(trifluoromethyl)-4,5,6,7-tetrahydro-1H-pyrazolo[3,4-b]pyridine

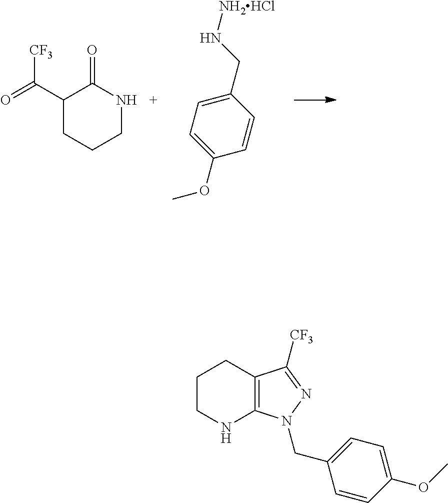

The mixture of 3-(2,2,2-trifluoroacetyl) piperidin-2-one (2500 mg, 12.8 mmol) and (4-methoxybenzyl) hydrazine hydrochloride (2685 mg, 15.4 mmol) in toluene (50 mL) was stirred at 60° C. for 1 h and concentrated. The solution of (2,4-bis(4-methoxyphenyl)-1,3-dithia-2,4-diphosphetane-2,4-disulfide) (10364 mg, 25.6 mmol) in THF (50 mL) was added and the mixture was stirred at 70° C. for 12 h. The mixture was concentrated, and the crude product was purified by silica-gel column chromatography (PE:EA=5:1) to get the title compound (1.90 g, 48% yield) as a yellow solid.

LC-MS (Method 1): Retention time=2.21 min. MS (ESI) m/z 312.0 [M+H]⁺.

### Step 2. Synthesis of 3-((1-(4-methoxybenzyl)-3-(trifluoromethyl)-1,4,5,6-tetrahydro-7H-pyrazolo[3,4-b]pyridine-7-yl)methyl)bicyclo[1.1.1]pentane-1-carboxylic acid

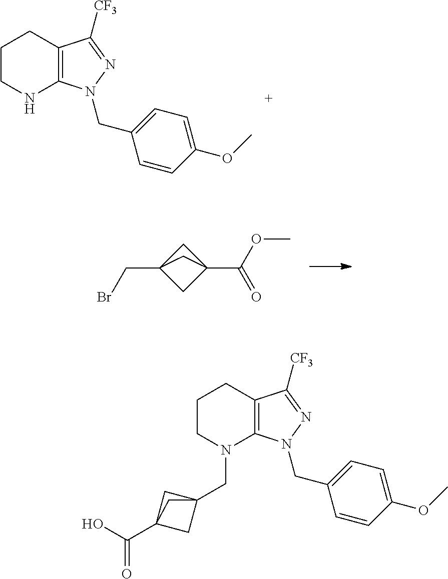 [SMILES: COc1ccc(Cn2nc(C(F)(F)F)c3c2NCCC3)cc1 ; COC(=O)C12CC(CBr)(C1)C2 ; COc1ccc(Cn2nc(C(F)(F)F)c3c2N(CC24CC(C(=O)O)(C2)C4)CCC3)cc1]

To the solution of 1-[(4-methoxyphenyl)methyl]-3-(trifluoromethyl)-4,5,6,7-tetrahydropyrazolo[3,4-b]pyridine (810 mg, 2.60 mmol) in THF (5 mL) were added methyl 3-(bromomethyl)bicyclo[1.1.1]pentane-1-carboxylate (570 mg, 2.60 mmol) and t-BuOK (7.8 mL, 7.81 mmol, 1 M in THF). The reaction was stirred at rt for 2 h. The mixture was concentrated, and the crude was purified by flash chromatography (10-50% EA in heptane) to get 3-[[1-[(4-methoxyphenyl)methyl]-3-(trifluoromethyl)-5,6-dihydro-4H-pyrazolo[3,4-b]pyridine-7-yl]methyl]bicyclo[1.1.1]pentane-1-carboxylic acid (820 mg, 72% yield) as a white solid.

LC-MS (Method 1): Retention time=2.08 min. MS (ESI) m/z 436.3 [M+H]⁺.

### Step 3. Synthesis of methyl 3-((1-(4-methoxybenzyl)-3-(trifluoromethyl)-1,4,5,6-tetrahydro-7H-pyrazolo[3,4-b]pyridine-7-yl)methyl)bicyclo[1.1.1]pentane-1-carboxylate

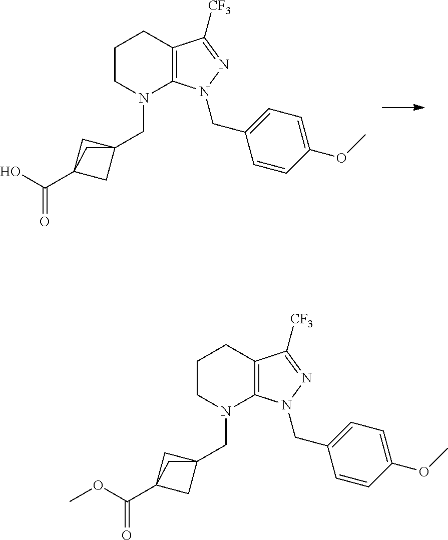

To the solution of 3-((1-(4-methoxybenzyl)-3-(trifluoromethyl)-1,4,5,6-tetrahydro-7H-pyrazolo[3,4-b]pyridine-7-yl)methyl)bicyclo[1.1.1]pentane-1-carboxylic acid (820 mg, 1.88 mmol) in methanol (5 mL) and THF (5 mL) was added trimethylsilyldiazomethane (1.9 mL, 3.77 mmol, 2 M) dropwise and the mixture was stirred at rt for 2 h. The mixture was concentrated. The crude product was purified by flash chromatography (10-20% EA in heptane) to get the title compound (790 mg, 93% yield) as a white solid.

LC-MS (Method 1): Retention time=2.25 min. MS (ESI) m/z 450.3 [M+H]⁺.

### Step 4. Synthesis of methyl 3-((3-(trifluoromethyl)-5,6-dihydro-1H-pyrazolo[3,4-b]pyridine-7 (4H)-yl)methyl)bicyclo[1.1.1]pentane-1-carboxylate

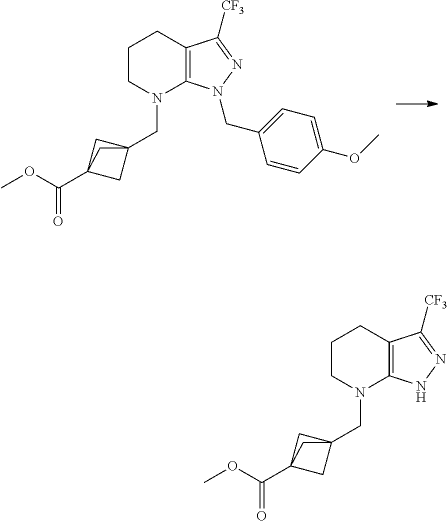

To the solution of methyl 3-((1-(4-methoxybenzyl)-3-(trifluoromethyl)-1,4,5,6-tetrahydro-7H-pyrazolo[3,4-b]pyridine-7-yl)methyl)bicyclo[1.1.1]pentane-1-carboxylate (470 mg, 1.05 mmol) in chloroform (4 mL) was added trifluoroacetic acid (2.0 mL, 26.0 mmol) and the mixture was stirred at 80° C. for 5 h. The mixture was concentrated. The crude product was purified by flash chromatography eluted with 10-20% ethyl acetate in heptane to yield the title compound (170 mg, 49% yield) as a white solid.

LC-MS (Method 4): Retention time=1.97 min. MS (ESI) m/z 330.2 [M+H]⁺. ¹H NMR (500 MHZ, DMSO-d₆) δ 12.42 (s, 1H), 3.57 (s, 3H), 3.29 (s, 2H), 3.17-3.10 (m, 2H), 2.46-2.50 (m, 2H), 1.92 (s, 6H), 1.85-1.77 (m, 2H).

The following intermediates were prepared following the similar protocol as Intermediate A-1.

| Code | Structure | IUPAC name | LCMS |
|---|---|---|---|
| Intermediate A-2 | 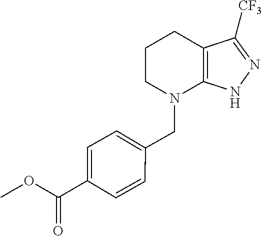 [SMILES: COC(=O)c1ccc(CN2CCCc3c(C(F)(F)F)n[nH]c32)cc1] | Methyl 4-((3-(trifluoromethyl)- 1,4,5,6-tetrahydro-7H- pyrazolo[3,4-b]pyridine-7- yl)methyl)benzoate | LC-MS (Method 1): Retention time = 1.75 min. MS (ESI) m/z 339.9 [M + H]⁺. |
| Intermediate A-3 | 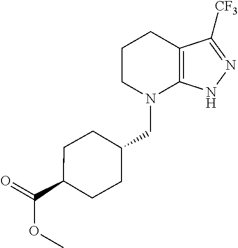 [SMILES: COC(=O)[C@H]1CC[C@H](CN2CCCc3c(C(F)(F)F)n[nH]c32)CC1] | Trans-Methyl 4-((3- (trifluoromethyl)-1,4,5,6- tetrahydro-7H-pyrazolo[3,4- b]pyridine-7- yl)methyl)cyclohexane-1- carboxylate | LC-MS (Method 2): Retention time = 2.08 min. MS (ESI) m/z 346.3 [M + H]⁺. |
| Intermediate A-4 | 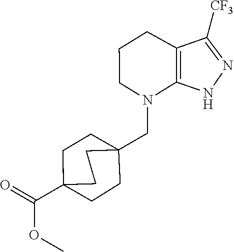 [SMILES: COC(=O)C12CCC(CN3CCCc4c(C(F)(F)F)n[nH]c43)(CC1)CC2] | Methyl 4-((3-(trifluoromethyl)- 1,4,5,6-tetrahydro-7H- pyrazolo[3,4-b]pyridine-7- yl)methyl)bicyclo[2.2.2]octane- 1-carboxylate | LC-MS (Method 2): Retention time = 1.95 min. MS (ESI) m/z 372.1 [M + H]⁺. |

### Preparation of Intermediate B-1

### 3-(3-(Trifluoromethyl)-4,5,6,7-tetrahydro-1H-pyrazolo[3,4-b]pyridine-1-yl) phenol

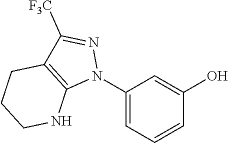 [SMILES: Oc1cccc(-n2nc(C(F)(F)F)c3c2NCCC3)c1]

### Step 1. Synthesis of 1-(3-bromophenyl)-3-(trifluoromethyl)-4,5,6,7-tetrahydropyrazolo[3,4-b]pyridine (Intermediate B-1a)

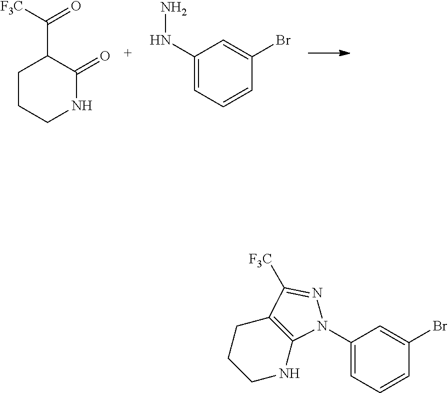 [SMILES: O=C1NCCCC1C(=O)C(F)(F)F ; NNc1cccc(Br)c1 ; FC(F)(F)c1nn(-c2cccc(Br)c2)c2c1CCCN2]

A solution of 3-(2,2,2-trifluoroacetyl) piperidin-2-one (1500 mg, 7.69 mmol) and (3-bromophenyl) hydrazine (2157 mg, 11.5 mmol) in toluene (10 mL) was stirred at 60° C. for 2 h, then the solvent was evaporated. The residue was dissolved in THF (20 mL). To this solution was added (2,4-bis(4-methoxyphenyl)-1,3-dithia-2,4-diphosphetane-2,4-disulfide) (4664 mg, 11.5 mmol). The reaction was stirred at 55° C. for 12 h. The reaction mixture was then concentrated in vacuum. The residue was purified by silica gel column chromatography (0-35% ethyl acetate in heptane) to yield the title compound B-1a (1200 mg, 43% yield) as a colorless oil.

LC-MS (Method 3): Retention time=2.26 min. MS (ESI) m/z 345/347 [M+H]⁺.

### Step 2. Synthesis of 3-[3-(trifluoromethyl)-4,5,6,7-tetrahydropyrazolo[3,4-b]pyridine-1-yl]phenol

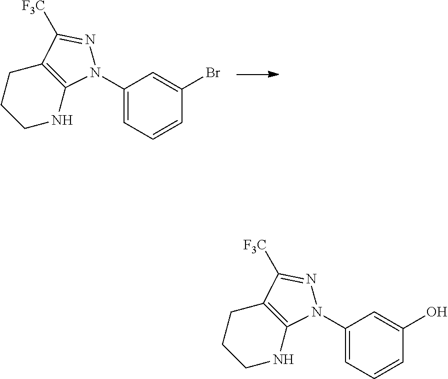 [SMILES: FC(F)(F)c1nn(-c2cccc(Br)c2)c2c1CCCN2 ; Oc1cccc(-n2nc(C(F)(F)F)c3c2NCCC3)c1]

To a solution of 1-(3-bromophenyl)-3-(trifluoromethyl)-4,5,6,7-tetrahydropyrazolo[3,4-b]pyridine (500 mg, 1.44 mmol) in 1,4-dioxane (5 mL) and water (0.5 mL) were added cesium hydroxide monohydrate (728 mg, 4.33 mmol) and 2-di-tert-butylphosphino-2′,4′,6′-triisopropylbiphenyl (31 mg, 0.0722 mmol) followed by tris(dibenzylideneacetone)dipalladium (66 mg, 0.0722 mmol) under Ar. The reaction mixture was stirred at 100° C. for 12 h. Then water (30 mL) was added and extracted with EA (2×40 mL). The combined organic extracts were washed with brine (30 mL), dried over Na₂SO₄, and then concentrated in vacuum. The residue was purified by Combi-flash (A: water (10 mmol/L NH₄HCO₃); B: ACN) to yield the title compound (300 mg, 73% yield) as a yellow solid.

LC-MS (Method 1): Retention time=1.98 min. MS (ESI) m/z 284.0 [M+H]⁺.

### Preparation of Intermediate B-2

### 5-(3-(Trifluoromethyl)-4,5,6,7-tetrahydro-1H-pyrazolo[3,4-b]pyridine-1-yl)pyridine-3-ol

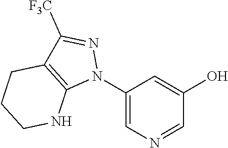 [SMILES: Oc1cncc(-n2nc(C(F)(F)F)c3c2NCCC3)c1]

The title compound was synthesized essentially in the same manner as Preparation of Intermediate B-1.

LC-MS (Method 1): Retention time=1.76 min. MS (ESI) m/z 285.0 [M+H]⁺.

### Preparation of Intermediate C-1

### 1-(2,2-difluorobenzo[d][1,3]dioxol-5-yl)ethan-1-ol

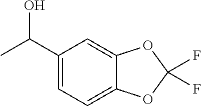

### Step 1. Synthesis of 1-(2,2-difluorobenzo[d][1,3]dioxol-5-yl)ethan-1-one

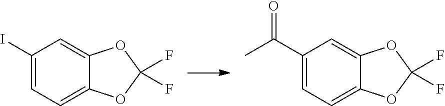

To the solution of 1-ethoxyvinyltri-n-butyltin (1.7 mL, 4.93 mmol) in toluene (20 mL) was added 2,2-difluoro-5-iodo-1,3-benzodioxole (1.40 g, 4.93 mmol) and Pd(PPh₃)₂Cl₂ (138 mg, 0.20 mmol). The reaction was stirred at 90° C. for 12 h under N₂, then cooled to rt. HCl (30 mL, 60.0 mmol) was added and stirred for 0.5 h. The mixture was extracted with EA (3×30 mL). The organic layers were combined, washed with brine (30 mL), dried over Na₂SO₄, filtered and concentrated. The crude product was purified by flash column chromatography eluted with 5% ethyl acetate in isohexane to get the title compound (950 mg, 95% yield) as a colorless oil.

LC-MS (Method 1): Retention time=2.02 min. MS (ESI) m/z 201.0 (M+H)⁺.

¹H NMR (500 MHZ, CDCl₃) δ 7.77 (dd, J=8.3, 1.6 Hz, 1H), 7.69 (d, J=1.6 Hz, 1H), 7.14 (d, J=8.3 Hz, 1H), 2.60 (s, 3H).

### Step 2. Synthesis of 1-(2,2-difluorobenzo[d][1,3]dioxol-5-yl)ethan-1-ol

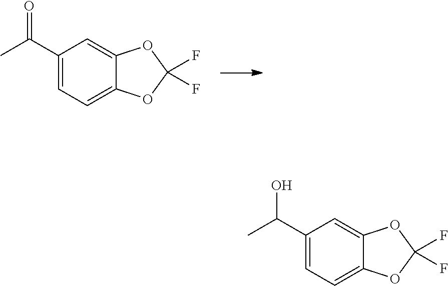

To a solution of 1-(2,2-difluoro-1,3-benzodioxol-5-yl)pyridine (900 mg, 4.50 mmol) in methanol (10 mL) was added NaBH₄ (340 mg, 8.99 mmol) at 0° C. and stirred for 2 h. Then the mixture was poured into water (40 mL) and extracted with EA (3×30 mL). The organic layers were combined, washed with brine (30 mL), dried over Na₂SO₄, filtered, and concentrated. The crude was purified by flash column chromatography eluted with 10% ethyl acetate in isohexane to get the title compound (860 mg, 94.6% yield) as a colorless oil.

LC-MS (Method 1): Retention time=1.94 min. MS (ESI) m/z 185.0 (M−H₂O+H)⁺.

### Preparation of Intermediate C-1R

### I-1-(2,2-difluorobenzo[d][1,3]dioxol-5-yl)ethan-1-ol

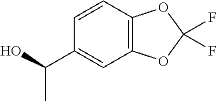

### Step 1. Synthesis of (R)-1-(2,2-difluorobenzo[d][1,3]dioxol-5-yl)ethan-1-ol

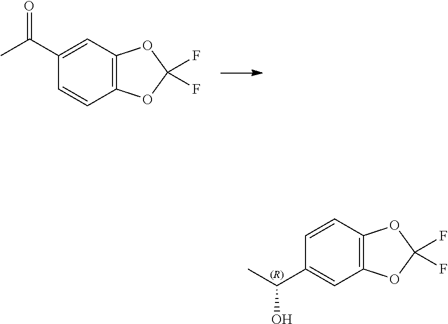

To a solution of(S)-2-methyl-CBSoxazaborolidine (Ref. 1) [(8.25 g, 0.03 mol) in DCM (200 mL) under N₂ was added BH₃: Me₂S (150 mL, 2 M in DCM) at 0° C. The reaction was stirred at room temperature for 0.5 h, and then the solution of 1-(2,2-difluorobenzo[d][1,3]dioxol-5-yl)ethan-1-one (product of Step 1, Preparation of Intermediate C-1, 60 g, 0.30 mol) in DCM (300 mL) was added dropwise at 0° C. for 1 h. The reaction mixture was stirred at room temperature for 4 h. The reaction was quenched by addition of MeOH at 0° C., and then concentrated in vacuo to give the residue which was purified by flash silica gel column chromatography (ethyl acetate in petroleum ether from 0% to 10%) to afford the title compound (56 g, 92% yield) as a light-yellow liquid. LC-MS: Retention time=1.75 min. MS (ESI) m/z 185.0 [M−H₂O+H]⁺.

### Preparation of Intermediate C-1S

### (S)-1-(2,2-difluorobenzo[d][1,3]dioxol-5-yl)ethan-1-ol

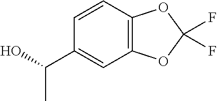

Intermediate C-1S was synthesized in essentially the same manner as Intermediate C-1R except using (R)-2-methyl-CBSoxazaborolidine (Ref. 1) in Step 1.

| | | | | Protocols |
|---|---|---|---|---|
| Code | Structure | IUPAC name | LCMS | followed |
| Intermediate C-2 | 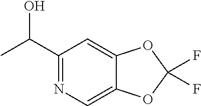 | 1-(2,2-difluoro- [1,3]dioxolo[4,5-c]pyridine-6- yl)ethan-1-ol | LC-MS: Retention time = 1.71 min. MS (ESI) m/z 204.1 [M + H]⁺. | Intermediate C-1 |
| Intermediate C-3 | 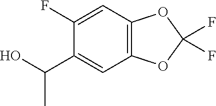 | 1-(2,2,6- trifluorobenzo[d][1,3]dioxol- 5-yl)ethan-1-ol | LC-MS: Retention time = 1.94 min. MS (ESI) m/z 202.9 [M − H₂O + H]⁺. | Intermediate C-1 |
| Intermediate C-3R | 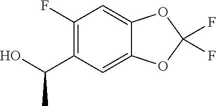 | (R)-1-(2,2,6- trifluorobenzo[d][1,3]dioxol- 5-yl)ethan-1-ol | LC-MS: Retention time = 1.94 min. MS (ESI) m/z 202.9 [M − H₂O + H]⁺. | Intermediate C-1R |
| Intermediate C-3S | 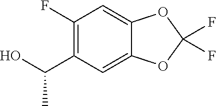 | (S)-1-(2,2,6- trifluorobenzo[d][1,3]dioxol- 5-yl)ethan-1-ol | Retention time = 1.94 min. MS (ESI) m/z 202.9 [M − H2O + H]⁺. | Intermediate C-1S |

### Preparation of Intermediate C-4

### 5-(1-chloroethyl)-2,2-difluorobenzo[d][1,3]dioxole

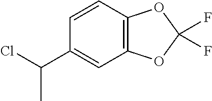

To a solution of 1-(2,2-difluoro-1,3-benzodioxol-5-yl)ethanol (Intermediate C-1) (1.00 eq, 6.00 g, 29.7 mmol) and triethylamine (3.00 eq, 12 mL, 89.0 mmol) in DCM (120 mL) was added methanesulfonyl chloride (1.50 eq, 3.5 mL, 44.5 mmol) at 0° C. The reaction was stirred at rt for 4 h. Then the mixture was diluted with water (50 mL) and extracted with DCM (3×80 mL). The combined organic phase was washed with brine and dried over Na₂SO₄. The organics were concentrated under vacuum. The residue was purified by flash column chromatography (PE:EA=5:1) to give 5-(1-chloroethyl)-2,2-difluoro-1,3-benzodioxole (5.00 g, 22.7 mmol, 76.3% yield) as a colorless oil.

LC-Mass Method 1: Retention time=2.15 min. MS (ESI) m/z: not observed.

### Preparation of Intermediate C-4R

### (R)-1-(2,2-difluorobenzo[d][1,3]dioxol-5-yl) propan-1-ol

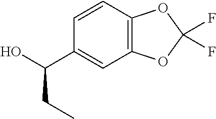

### Step 1. Synthesis of 1-(2,2-difluoro-1,3-benzodioxol-5-yl) propan-1-ol

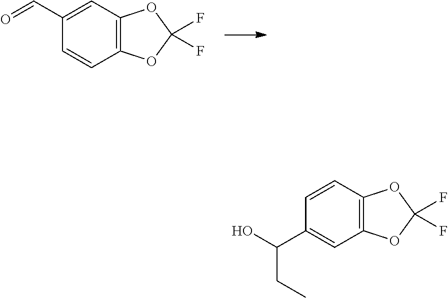

To a solution of 2,2-difluoro-1,3-benzodioxole-5-carbaldehyde (3000 mg, 16.1 mmol) in THF (30 mL) was added ethylmagnesium bromide (48 mL, 48.4 mmol). The reaction was stirred at rt for 1 h. Then quenched with NH₄Claq, extracted with EA (10 mL×3). The combined organic solution was washed with brine (10 mL), dried over Na₂SO₄, filtered, and concentrated in vacuum. The residue was purified by silica gel column chromatography (0-10% ethyl acetate in heptane) to yield the title compound (2500 mg, 71.7% yield) as a colorless oil.

LCMS (Method 4): Retention time=1.80 min. MS (ESI) m/z 199.1 [M+H−H₂O]⁺.

### Step 2. Synthesis of 1-(2,2-difluoro-1,3-benzodioxol-5-yl) propan-1-one

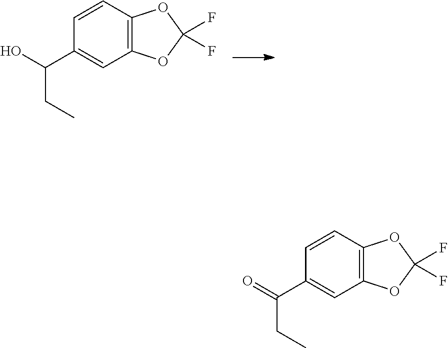

To a solution of 1-(2,2-difluoro-1,3-benzodioxol-5-yl) propan-1-ol (2500 mg, 11.6 mmol) in DCM (30 mL) was added manganese dioxide (5027 mg, 57.8 mmol). The suspension was stirred at rt for 12 h. Then the solid was filtered off. The filtrate was concentrated in vacuum. The residue was purified by silica gel column chromatography (0-5% ethyl acetate in heptane) to yield the title compound (2000 mg, 80.7% yield) as a colorless oil. LCMS (Method 4): Retention time=1.94 min. MS (ESI) m/z: Not observed.

### Step 3. Synthesis of (R)-1-(2,2-difluorobenzo[d][1,3]dioxol-5-yl) propan-1-ol

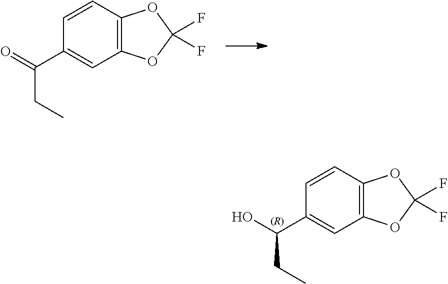

To a solution of 1-(2,2-difluoro-1,3-benzodioxol-5-yl) propan-1-one (2200 mg, 10.30 mmol) and(s)-methyl oxazaborolidine (0.31 mL, 1.03 mmol) in DCM (25 mL) under N₂ was added borane-methyl sulfide complex (0.98 mL, 10.30 mmol). The reaction was stirred at room temperature for 4 h. The reaction mixture was quenched by addition of MeOH at 0° C., and then concentrated in vacuum to give a residue which was purified by flash silica gel column chromatography (ethyl acetate in petroleum ether from 0% to 10%) to afford the title compound (2000 mg, 88.3% yield) as a colorless liquid.

LCMS (Method 2): Retention time=1.83 min. MS (ESI) m/z 199.1 [M−H₂O+H]⁺.

### Preparation of Intermediate C-6a

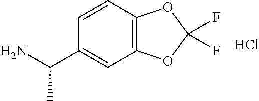

### Step 1. Synthesis of (R,E)-N-((2,2-difluorobenzo[d][1,3]dioxol-5-yl)methylene)-2-methylpropane-2-sulfinamide

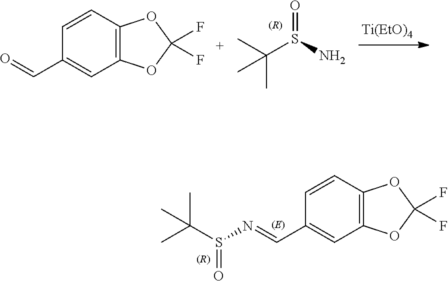

To a solution of R-2-methylpropane-2-sulfinamide (SM-1) (3256 mg, 26.9 mmol) in THF (50 mL) was added 2,2-difluoro-1,3-benzodioxole-5-carbaldehyde (5000 mg, 26.9 mmol) and titanium (IV) isopropoxide (16 mL, 53.7 mmol). The reaction was stirred at 60° C. for 2 h. Then the solid was filtered off. The filtrate was concentrated in vacuum. The residue was purified by silica gel column chromatography (0-20% ethyl acetate in heptane) to yield the title compound (5800 mg, 74.6% yield) as a yellow oil.

LCMS (Method 4): Retention time=2.09 min. MS (ESI) m/z 290.0 [M+H]⁺.

### Step 2. Synthesis of N—((S)-1-(2,2-difluorobenzo[d][1,3]dioxol-5-yl)ethyl)-2-methylpropane-2-sulfinamide

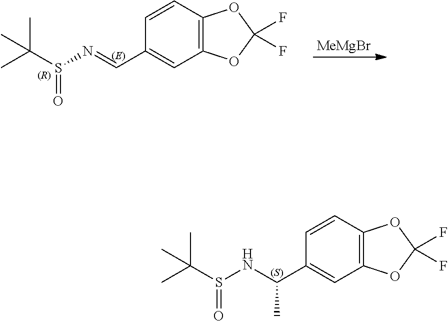

To a solution of (R,E)-N-((2,2-difluorobenzo[d][1,3]dioxol-5-yl)methylene)-2-methylpropane-2-sulfinamide (5800 mg, 20.0 mmol) in THF (60 mL) was added methyl magnesium bromide (20 mL, 60.1 mmol) under N₂ at −20° C. The reaction was stirred at rt for 1 h. Then the reaction was quenched with NH₄Claq and extracted with EA (10 mL×3). The EA solution was washed with brine (10 mL), dried over Na₂SO₄, filtered, and concentrated in vacuum. The residue was purified by silica gel column chromatography (0-10% ethyl acetate in heptane) to yield the title compound (4500 mg, 73.5% yield) as a yellow oil.

LCMS (Method 4): Retention time=1.91 min. MS (ESI) m/z 306.1 [M+H]⁺.

### Step 3. Synthesis of(S)-1-(2,2-difluorobenzo[d][1,3]dioxol-5-yl)ethan-1-amine

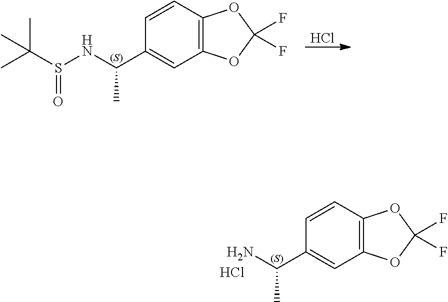

To a solution of N-[(1S)-1-(2,2-difluoro-1,3-benzodioxol-5-yl)ethyl]-2-methyl-propane-2-sulfinamide (4500 mg, 14.7 mmol) in 1,4-dioxane (18 mL) was added hydrochloric acid (18 mL, 73.7 mmol). The solution was stirred at rt for 2 h. The solvent was removed. To this residue was added EA (20 mL) and concentrated in vacuum. Another portion of EA (20 mL) was added and concentrated again to yield the title compound (3000 mg, 85.7% yield) as a white solid.

LCMS (Method 4): Retention time=1.60 min. MS (ESI) m/z 185.1 [M+H−NH₂]⁺.

### Preparation of Intermediate C-6b

### (S)-1-(2,2-difluorobenzo[d][1,3]dioxol-5-yl)-2-methylpropan-1-amine hydrogen chloride

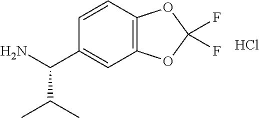 [SMILES: CC(C)[C@H](N)c1ccc2c(c1)OC(F)(F)O2 ; Cl]

The Intermediate C-6b was synthesized essentially in the same manner as Intermediate C-6a in 86.9% yield as a white solid.

LCMS (Method 4): Retention time=1.86 min. MS (ESI) m/z 213.1 [M−H₂O+H]⁺.

### Preparation of Intermediate C-6c

### (S)-cyclopropyl(2,2-difluorobenzo[d][1,3]dioxol-5-yl)methanamine hydrogen chloride

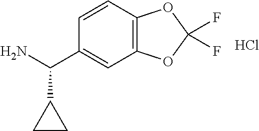 [SMILES: N[C@H](c1ccc2c(c1)OC(F)(F)O2)C1CC1 ; Cl]

The Intermediate C-6c was synthesized essentially in the same manner as Intermediate C-6a in 84% yield as a white solid.

LCMS (Method 4): Retention time=1.75 min. MS (ESI) m/z 211.1 [M−NH₃+H]⁺.

### Preparation of Intermediate C-6d

### (S)-1-(2,2,6-trifluorobenzo[d][1,3]dioxol-5-yl)ethan-1-amine

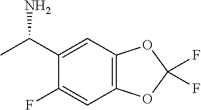

### Step 1. Synthesis of 4-bromo-5-fluorobenzene-1,2-diol

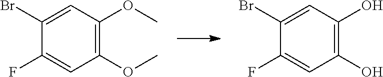

To a solution of 1-bromo-2-fluoro-4,5-dimethoxy-benzene (1.00 eq, 15.00 g, 63.8 mmol) in DCM (200 mL) was added boron tribromide (1.50 eq, 9.0 mL, 95.7 mmol, 1M in dichloromethane) at 0° C. The reaction was stirred at room temperature for 4 h. To the mixture was added methanol (50 mL) at 0° C. and the solvent evaporated under reduced pressure. The residue was purified by silica gel column chromatography eluted with DCM. The desired fractions were combined and concentrated to dryness in vacuo to obtain 4-bromo-5-fluoro-benzene-1,2-diol (13.00 g, 62.8 mmol, 98.4% yield) as a brown oil.

LC-MS Method 1: Retention time=1.50 min. MS (ESI) m/z 207.0, 209.0 [M+H]⁺.

### Step 2. Synthesis of 5-bromo-6-fluorobenzo[d][1,3]dioxole-2-thione

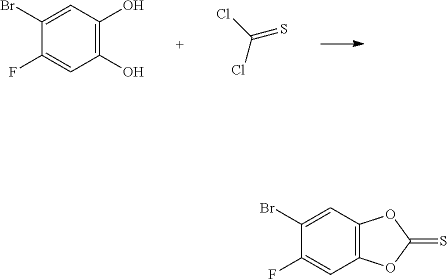

To a solution of 4-bromo-5-fluoro-benzene-1,2-diol (1.00 eq, 15.00 g, 72.5 mmol) and 4-dimethylaminopyridine (4.00 eq, 35412 mg, 290 mmol) in DCM (150 mL) was added thiophosgene (2.50 eq, 14 mL, 181 mmol) at 0° C. The reaction was stirred at room temperature for 12 h. The mixture was filtered and the solvent was evaporated under reduced pressure. The residue was purified by silica gel column chromatography eluted with PE/EA (10:1). The desired fractions were concentrated to dryness in vacuo to obtain 5-bromo-6-fluoro-1,3-benzodioxole-2-thione (15.00 g, 60.2 mmol, 83.1% yield) as a white solid.

LC-MS Method 2: Retention time=1.86 min. MS (ESI) m/z 249.0, 251 [M+H]⁺.

### Step 3. Synthesis of 5-bromo-2,2,6-trifluorobenzo[d][1,3]dioxole

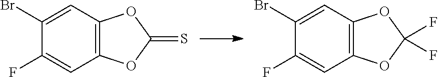 [SMILES: Fc1cc2oc(=S)oc2cc1Br ; Fc1cc2c(cc1Br)OC(F)(F)O2]

To a suspension of 1,3-dibromo-5,5-dimethylhydantoin (3.00 eq, 51660 mg, 181 mmol) in DCM (150 mL) was added hydrogen fluoride-pyridine (3.00 eq, 17907 mg, 181 mmol) (70%) at −78° C. under nitrogen. The reaction was stirred for 30 minutes. To this solution was added a solution of 5-bromo-6-fluoro-1,3-benzodioxole-2-thione (1.00 eq, 15.00 g, 60.2 mmol) in DCM (50 mL) at −78° C. The reaction was stirred at room temperature for 12 h. The organics were washed with water (200 mL), dried over Na₂SO₄, filtered, and concentrated under reduced pressure. The residue was purified by silica gel column chromatography eluted with PE. The desired fractions were concentrated to dryness in vacuo to obtain 5-bromo-2,2,6-trifluoro-1,3-benzodioxole (12.00 g, 47.1 mmol, 78.1% yield) as a colorless oil.

LC-MS Method 1: Retention time=2.13 min. MS (ESI) m/z 255.0, 257 [M+H]⁺.

### Step 4. Synthesis of 1-(2,2,6-trifluorobenzo[d][1,3]dioxol-5-yl)ethan-1-one

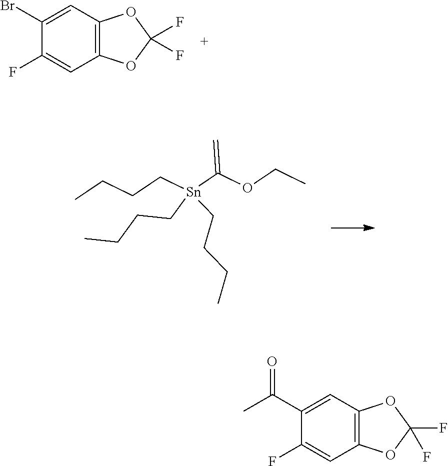 [SMILES: Fc1cc2c(cc1Br)OC(F)(F)O2 ; C=[C](OCC)[Sn]([CH2]CCC)([CH2]CCC)[CH2]CCC ; CC(=O)c1cc2c(cc1F)OC(F)(F)O2]

To a solution of 5-bromo-2,2,6-trifluoro-1,3-benzodioxole (1.00 eq, 12.00 g, 47.1 mmol) and 1-ethoxyvinyltri-n-butyltin (1.00 eq, 16 mL, 47.1 mmol) in toluene (100 mL) was added bis(triphenylphosphine) palladium (II) chloride (0.0363 eq, 1200 mg, 1.71 mmol) under nitrogen. The reaction was stirred at 90° C. for 12 h. To the mixture was added 2M HCl (100 mL) at 0° C., stirred for 30 minutes, and then extracted with EA (100 mL). The organics were washed with brine (100 mL), dried over Na₂SO₄, filtered, and concentrated under reduced pressure. The residue was purified by flash column chromatography eluted with PE/EA (10:1). The desired fractions were concentrated to dryness in vacuo to obtain 1-(2,2,6-trifluoro-1,3-benzodioxol-5-yl)pyridine (8.00 g, 36.7 mmol, 77.9% yield) as a colorless oil.

LC-MS Method 1: Retention time=1.85 min. MS (ESI) m/z 219.0 [M+H]⁺.

### Step 5. Synthesis of(S)-2-methyl-N—((S)-1-(2,2,6-trifluorobenzo[d][1,3]dioxol-5-yl)ethyl) propane-2-sulfinamide

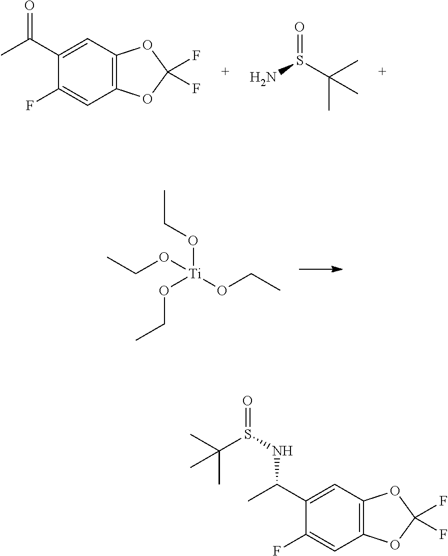

To a solution of 1-(2,2,6-trifluoro-1,3-benzodioxol-5-yl)pyridine (1.00 eq, 5.00 g, 22.9 mmol) and(S)-2-methylpropane-2-sulfinamide (1.50 eq, 4167 mg, 34.4 mmol) in THF (50 mL) was added tetraethoxytitanium (2.00 eq, 10458 mg, 45.8 mmol). The reaction was stirred at 60° C. for 12 h. Sodium borohydride (1.10 eq, 954 mg, 25.2 mmol) was added portion wise to the mixture at 0° C. The reaction was stirred at 0° C. for 30 min. To the mixture was added water (20 mL) at 0° C., stirred for 30 min, and filtered. The filtrate was diluted with EA (50 ml). The organic solution was washed with brine (50 mL), dried over Na₂SO₄, filtered, and concentrated under reduced pressure. The residue was purified by flash column chromatography eluted with PE/EA (2:1). The desired fractions were concentrated to dryness in vacuo to obtain the title compound (5.00 g, 15.5 mmol, 67.5% yield) as a white solid.

LC-MS Method 2: Retention time=1.85 min. MS (ESI) m/z 324.0 [M+H]⁺.

### Step 6. Synthesis of(S)-1-(2,2,6-trifluorobenzo[d][1,3]dioxol-5-yl)ethan-1-amine

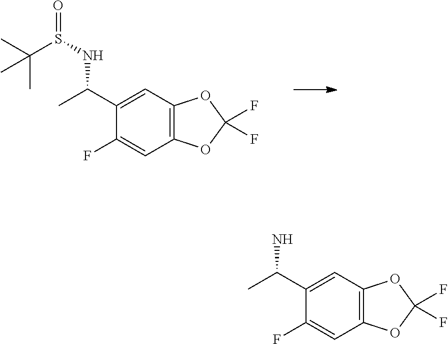 [SMILES: C[C@H](N[S@@](=O)C(C)(C)C)c1cc2c(cc1F)OC(F)(F)O2 ; C[C@H](N)c1cc2c(cc1F)OC(F)(F)O2]

To a solution of(S)-2-methyl-N—((S)-1-(2,2,6-trifluorobenzo[d][1,3]dioxol-5-yl)ethyl) propane-2-sulfinamide (1.00 eq, 5.00 g, 15.5 mmol) in methanol (20 mL) was added hydrogen chloride (4.0 M in 1,4-dioxane, 20 mL). The reaction was stirred at room temperature for 30 min. The mixture was concentrated under reduced pressure. The residue was dissolved in Na₂CO₃aq (20 mL) and extracted with EA (50 mL). The EA solution was washed with brine (20 mL), dried over Na₂SO₄, filtered, and concentrated to dryness in vacuo to obtain (1S)-1-(2,2,6-trifluoro-1,3-benzodioxol-5-yl)ethanamine (3.20 g, 14.6 mmol, 94.4% yield) as a yellow oil.

LC-MS Method 1: Retention time=1.22 min. MS (ESI) m/z 203.3 [M+H—NH_{2]}⁺.

### Preparation of Intermediate D-1

### 3-(Trifluoromethyl)-1,4,5,6-tetrahydro-7H-indazol-7-one

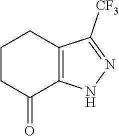 [SMILES: O=C1CCCc2c(C(F)(F)F)n[nH]c21]

### Step 1. Synthesis of 3-(trifluoromethyl)-1,4,5,6-tetrahydro-7H-indazol-7-one

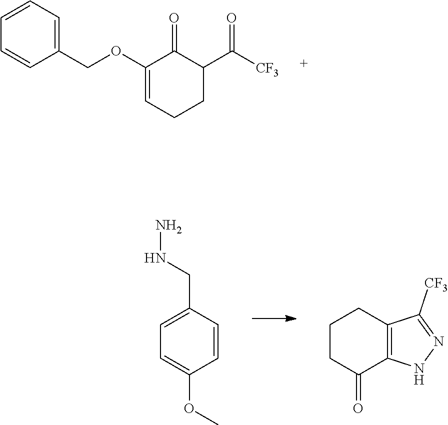 [SMILES: O=C1C(OCc2ccccc2)=CCCC1C(=O)C(F)(F)F ; COc1ccc(CNN)cc1 ; O=C1CCCc2c(C(F)(F)F)n[nH]c21]

To a solution of 2-(benzyloxy)-6-(2,2,2-trifluoroacetyl)cyclohex-2-en-1-one (6000 mg, 20.0 mmol) and (4-methoxybenzyl) hydrazine (3041 mg, 20.0 mmol) in ethanol (60 mL) was added triethylamine (2.8 mL, 20.0 mmol). The reaction was stirred at 80° C. for 8 h. To this reaction mixture was added TFA (20 mL) at 80° C. and stirred for 2 h. The reaction mixture was concentrated in vacuo. The residue was purified by flash chromatography on silica gel (ethyl acetate in petroleum ether from 0% to 20%) to afford the crude product which was further purified by prep-HPLC (NH₄HCO₃ as modifier) to afford the title compound (1400 mg, 34% yield) as a white solid.

LC-MS (Method 2): Retention time=1.46 min. MS (ESI) m/z 204.9 [M+H]⁺.

### Preparation of Intermediate D-1b

### 1-(3-bromo-4-fluorophenyl)-7-oxo-4,5,6,7-tetrahydro-1H-indazole-3-carboxylic acid

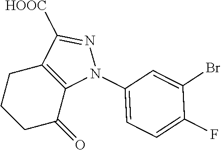 [SMILES: O=C(O)c1nn(-c2ccc(F)c(Br)c2)c2c1CCCC2=O]

### Step 1. Synthesis of ethyl 2-(3-(benzyloxy)-2-oxocyclohex-3-en-1-yl)-2-oxoacetate

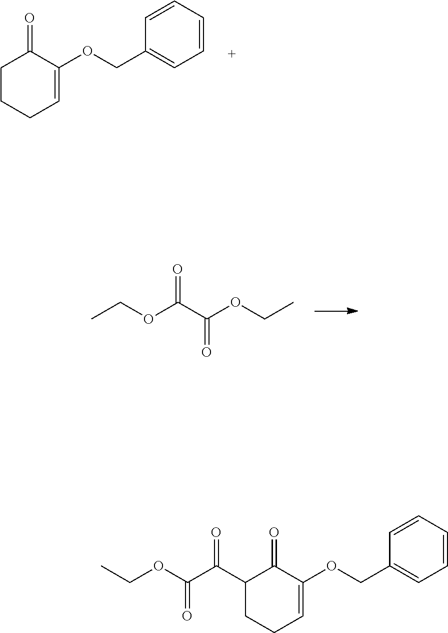

To a solution of 2-benzyloxycyclohex-2-en-1-one (1.00 eq, 5000 mg, 24.7 mmol) in THF (50 mL) was added lithium bis(trimethylsilyl)amide (1.50 eq, 6205 mg, 37.1 mmol) slowly at −50° C. The solution was stirred at rt for 0.5 h. Then diethyl oxalate (1.20 eq, 4.1 mL, 29.7 mmol) was added slowly and the mixture was stirred at rt overnight. The reaction was quenched with NH₄Claq and the aqueous was extracted with EA (10 mL×3). The combined organic solution was washed with brine (10 mL), dried over Na₂SO₄, and concentrated in vacuum. The residue was purified by silica gel column chromatography (0-15% ethyl acetate in heptane) to yield the title compound ethyl 2-(3-benzyloxy-2-oxo-cyclohex-3-en-1-yl)-2-oxo-acetate (6000 mg, 19.8 mmol, 80.3% yield) as a yellow oil.

LC-MS (Method 2): Retention time=1.26 min. MS (ESI) m/z 303.1 [M+H]⁺.

### Step 2. Synthesis of ethyl 1-(3-bromo-4-fluorophenyl)-7-oxo-4,5,6,7-tetrahydro-1H-indazole-3-carboxylate

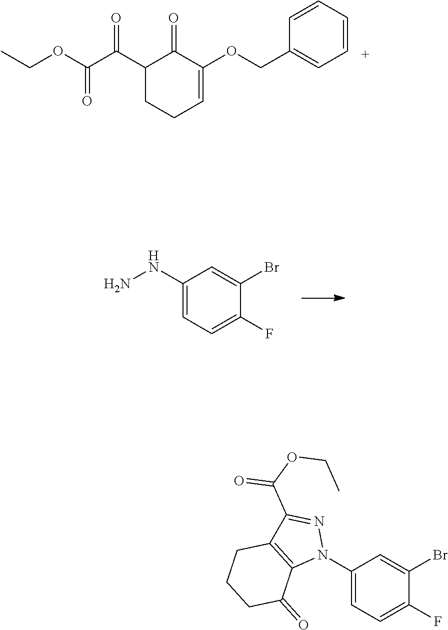 [SMILES: CCOC(=O)C(=O)C1CCC=C(OCc2ccccc2)C1=O ; NNc1ccc(F)c(Br)c1 ; CCOC(=O)c1nn(-c2ccc(F)c(Br)c2)c2c1CCCC2=O]

To a solution of ethyl 2-(3-benzyloxy-2-oxo-cyclohex-3-en-1-yl)-2-oxo-acetate (1.00 eq, 3000 mg, 9.92 mmol) in ethanol (30 mL) was added (3-bromo-4-fluoro-phenyl) hydrazine (1.10 eq, 2238 mg, 10.9 mmol). The reaction was stirred at 80° C. overnight. The solution was cooled to rt while yellow solid was precipitated. The solid was filtered and dried in vacuum to yield the title compound ethyl 1-(3-bromo-4-fluoro-phenyl)-7-oxo-5,6-dihydro-4H-indazole-3-carboxylate (1800 mg, 4.72 mmol, 47.6% yield) as a yellow solid.

LC-MS (Method 2): Retention time=2.01 min. MS (ESI) m/z 381.0, 383 [M+H]⁺.

### Step 3. Synthesis of 1-(3-bromo-4-fluorophenyl)-7-oxo-4,5,6,7-tetrahydro-1H-indazole-3-carboxylic acid

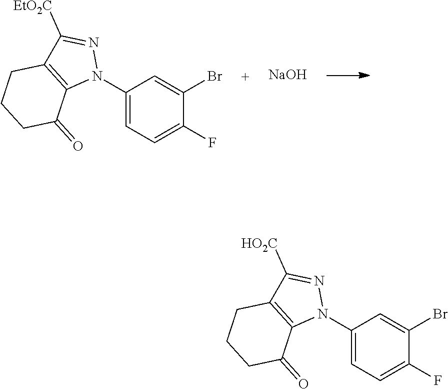 [SMILES: CCOC(=O)c1nn(-c2ccc(F)c(Br)c2)c2c1CCCC2=O ; "NaOH" ; O=C(O)c1nn(-c2ccc(F)c(Br)c2)c2c1CCCC2=O]

To a solution of ethyl 1-(3-bromo-4-fluoro-phenyl)-7-oxo-5,6-dihydro-4H-indazole-3-carboxylate (1.00 eq, 800 mg, 2.10 mmol) in methanol (10 mL) was added sodium hydroxide (3.00 eq, 252 mg, 6.30 mmol). The solution was stirred at rt for 2 h. Then solvent was removed and the pH was adjusted to <2 with 2N HCl. The aqueous solution was extracted with EA (25 mL×3). The organic solution was washed with brine (20 mL), dried over Na₂SO₄, concentrated in vacuum to yield the title compound 1-(3-bromo-4-fluoro-phenyl)-7-oxo-5,6-dihydro-4H-indazole-3-carboxylic acid (610 mg, 1.73 mmol, 82.3% yield) as a yellow solid.

LC-MS (Method 2): Retention time=1.24 min. MS (ESI) m/z 352.9, 354.9 [M+H]⁺.

### Preparation of Intermediate D-1c

### 1-(3-bromo-4-fluorophenyl)-7-oxo-4,5,6,7-tetrahydro-1H-indazole-3-carbonitrile

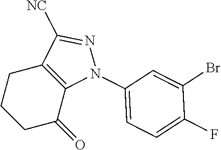 [SMILES: N#Cc1nn(-c2ccc(F)c(Br)c2)c2c1CCCC2=O]

### Step 1. Synthesis of 1-(3-bromo-4-fluorophenyl)-7-oxo-4,5,6,7-tetrahydro-1H-indazole-3-carboxamide

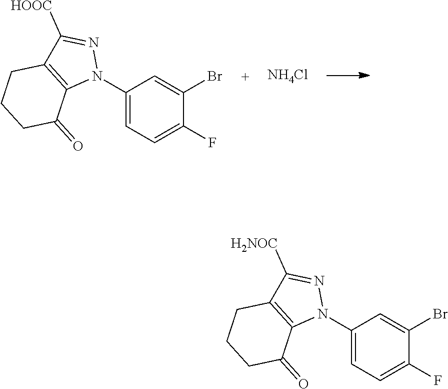 [SMILES: O=C(O)c1nn(-c2ccc(F)c(Br)c2)c2c1CCCC2=O ; "NH4Cl" ; NC(=O)c1nn(-c2ccc(F)c(Br)c2)c2c1CCCC2=O]

To a solution of 1-(3-bromo-4-fluoro-phenyl)-7-oxo-5,6-dihydro-4H-indazole-3-carboxylic acid (1.00 eq, 600 mg, 1.70 mmol) in DMF (5 mL) were added O-(7-azabenzotriazol-1-yl)-N,N,N′,N′-tetramethyluronium hexafluorophosphate (1.50 eq, 969 mg, 2.55 mmol), N,N-diisopropylethylamine (3.00 eq, 0.89 mL, 5.10 mmol), and ammonium chloride (2.00 eq, 182 mg, 3.40 mmol). The reaction was stirred at rt overnight. Then quenched with NH₄Claq (20 mL). The aqueous solution was extracted with EA (25 mL×3). The combined organic solution was washed with brine (20 mL), dried over Na₂SO₄, and concentrated in vacuum. The residue was purified by silica gel column chromatography (0-50% ethyl acetate in heptane) to yield the title compound 1-(3-bromo-4-fluoro-phenyl)-7-oxo-5,6-dihydro-4H-indazole-3-carboxamide (420 mg, 1.19 mmol, 70.2% yield) as a yellow solid.

LC-MS (Method 2): Retention time=1.67 min. MS (ESI) m/z 351.9, 353.9 [M+H]⁺.

### Step 2. Synthesis of 1-(3-bromo-4-fluorophenyl)-7-oxo-4,5,6,7-tetrahydro-1H-indazole-3-carbonitrile

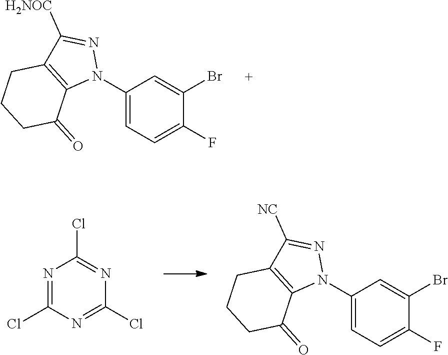 [SMILES: NC(=O)c1nn(-c2ccc(F)c(Br)c2)c2c1CCCC2=O ; Clc1nc(Cl)nc(Cl)n1 ; N#Cc1nn(-c2ccc(F)c(Br)c2)c2c1CCCC2=O]

To a solution of 1-(3-bromo-4-fluoro-phenyl)-7-oxo-5,6-dihydro-4H-indazole-3-carboxamide (1.00 eq, 420 mg, 1.19 mmol) in DMF (5 mL) was added 2,4,6-trichloro-1,3,5-triazine (2.00 eq, 440 mg, 2.39 mmol) at 0° C. and stirred for 1 h. Then water (20 mL) was added and extracted with EA (25 mL×3). The combined organic solution was washed with brine (20 mL), dried over Na₂SO₄, and concentrated in vacuum. The residue was purified by silica gel column chromatography (0-40% ethyl acetate in heptane) to yield 1-(3-bromo-4-fluoro-phenyl)-7-oxo-5,6-dihydro-4H-indazole-3-carbonitrile (360 mg, 1.08 mmol, 90.3% yield) as a white solid.

LC-MS (Method 2): Retention time=1.97 min. MS (ESI) m/z 333.7, 335.7 [M+H]⁺.

### Preparation of Intermediate D-1d

### 1-(4-fluoro-3-hydroxyphenyl)-7-oxo-4,5,6,7-tetrahydro-1H-indazole-3-carbonitrile

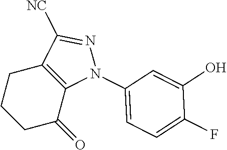 [SMILES: N#Cc1nn(-c2ccc(F)c(O)c2)c2c1CCCC2=O]

### Step 1. Synthesis of 1-(4-fluoro-3-hydroxy-phenyl)-7-oxo-5,6-dihydro-4H-indazole-3-carbonitrile

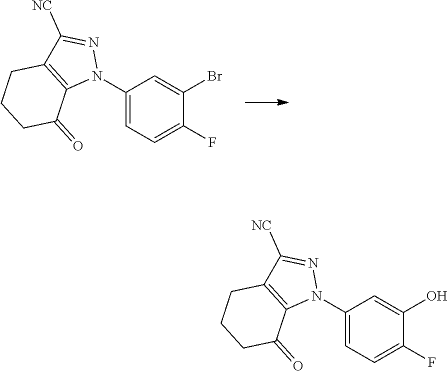 [SMILES: N#Cc1nn(-c2ccc(F)c(Br)c2)c2c1CCCC2=O ; N#Cc1nn(-c2ccc(F)c(O)c2)c2c1CCCC2=O]

To a solution of 1-(3-bromo-4-fluoro-phenyl)-7-oxo-5,6-dihydro-4H-indazole-3-carbonitrile (Intermediate D-1d) (360 mg, 1.08 mmol) in 1,4-dioxane (3 mL) were added cesium carbonate (878 mg, 2.69 mmol), water (58 mg, 3.23 mmol), and [9-[ditert-butyl-[6-methoxy-3-methyl-2-(2,4,6-triisopropylphenyl)phenyl]-25-phosphanyl]-8-aza-924-palladatricyclo[8.4.0.02,7]tetradeca-1 (10),2,4,6,11,13-hexaen-9-yl]methanesulfonate (90 mg, 0.11 mmol) at rt under N₂. The reaction was stirred at 100° C. for 1 h. The mixture was concentrated and purified by reverse phase combi-flash (0.01N NH₄HCO₃ in ACN) to yield 1-(4-fluoro-3-hydroxy-phenyl)-7-oxo-5,6-dihydro-4H-indazole-3-carbonitrile (150 mg, 51.3%) as a yellow solid.

LCMS (Method 4): R^{†}1.55 min. MS (ESI) m/z 272.1 [M+H]⁺.

### Preparation of Intermediate D-2

### 1-(3-hydroxyphenyl)-3-(trifluoromethyl)-1,4,5,6-tetrahydro-7H-indazol-7-one

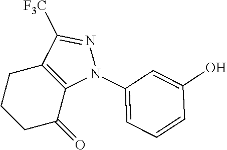 [SMILES: O=C1CCCc2c(C(F)(F)F)nn(-c3cccc(O)c3)c21]

### Step 1. Synthesis of 1-(3-bromophenyl)-3-(trifluoromethyl)-1,4,5,6-tetrahydro-7H-indazol-7-one (Intermediate D-2a)

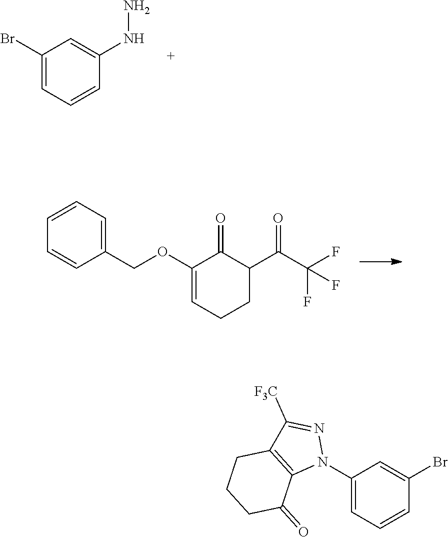 [SMILES: NNc1cccc(Br)c1 ; O=C1C(OCc2ccccc2)=CCCC1C(=O)C(F)(F)F ; O=C1CCCc2c(C(F)(F)F)nn(-c3cccc(Br)c3)c21]

To the solution of 2-benzyloxy-6-(2,2,2-trifluoroacetyl)cyclohex-2-en-1-one (7.90 g, 26.5 mmol) in DMF (80 mL) was added (3-bromophenyl) hydrazine (4.95 g, 26.5 mmol). The reaction was stirred at rt overnight under Ar. Then the mixture was diluted with water (800 mL) and extracted with EA (150 mL×3). The combined organic layers were washed with brine (300 mL) and dried over Na₂SO₄. The residue was concentrated under vacuum and purified by flash chromatography (Biotage, 120 g silica gel column @ 80 mL/min, eluted with 0-30% ethyl acetate in petroleum for 20 min) to yield the title compound Intermediate D-2a (4.81 g, 51% yield) as a light-yellow oil.

LC-MS (Method 1): Retention time=2.23 min. MS (ESI) m/z 359/361 [M+H]⁺.

### Step 2. Synthesis of 1-(3-hydroxyphenyl)-3-(trifluoromethyl)-1,4,5,6-tetrahydro-7H-indazol-7-one

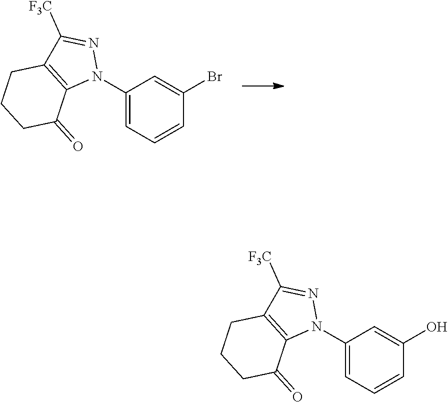 [SMILES: O=C1CCCc2c(C(F)(F)F)nn(-c3cccc(Br)c3)c21 ; O=C1CCCc2c(C(F)(F)F)nn(-c3cccc(O)c3)c21]

The title compound was prepared by following the protocol as described for Preparation of Intermediate D-1d.

LC-MS (Method 2): Retention time=1.91 min. MS (ESI) m/z 297.2 [M+H]⁺.

### Preparation of Intermediate D-2b

### 1-(3-bromophenyl)-3-(trifluoromethyl)-4,5,6,7-tetrahydroindazol-7-ol

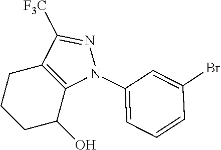 [SMILES: OC1CCCc2c(C(F)(F)F)nn(-c3cccc(Br)c3)c21]

### Step 1. Synthesis of 1-(3-bromophenyl)-3-(trifluoromethyl)-4,5,6,7-tetrahydroindazol-7-ol

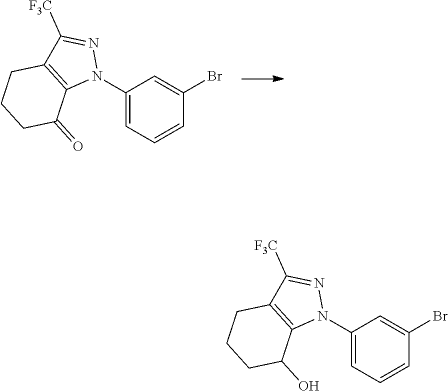 [SMILES: O=C1CCCc2c(C(F)(F)F)nn(-c3cccc(Br)c3)c21 ; OC1CCCc2c(C(F)(F)F)nn(-c3cccc(Br)c3)c21]

To a solution of 1-(3-bromophenyl)-3-(trifluoromethyl)-5,6-dihydro-4H-indazol-7-one (1.00 eq, 14.00 g, 39.0 mmol) in ethanol (100 mL) was added borane·sodium hydride (3.00 eq, 4.42 g, 117 mmol) at 0° C. under N₂. The reaction was stirred at rt under N₂ for 1 h. To the mixture was added NH₄Claq (30 mL) and water (100 mL). The mixture was extracted with EA (70 mL×3). The EA layers were washed with brine (80 mL), concentrated in vacuo. The crude product was then purified by flash column chromatography eluting 20% ethyl acetate in isohexane to get 1-(3-bromophenyl)-3-(trifluoromethyl)-4,5,6,7-tetrahydroindazol-7-ol (13.00 g, 36.0 mmol, 92.3% yield) as white solid.

### Preparation of Intermediate D-3

### 1-(4-fluoro-3-hydroxyphenyl)-3-(trifluoromethyl)-1,4,5,6-tetrahydro-7H-indazol-7-one

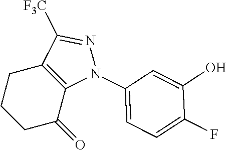 [SMILES: O=C1CCCc2c(C(F)(F)F)nn(-c3ccc(F)c(O)c3)c21]

### Step 1. Synthesis of (3-bromo-4-fluorophenyl) hydrazine

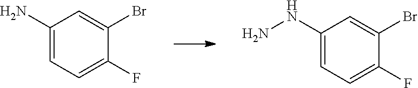 [SMILES: Nc1ccc(F)c(Br)c1 ; NNc1ccc(F)c(Br)c1]

To a solution of 3-bromo-4-fluoro-aniline (10000 mg, 52.6 mmol) in concentrated aqueous HCl (100 mL), was added sodium nitrite (4358 mg, 63.2 mmol) in portions at −10° C. The reaction was stirred at −10° C. for 1 h. Then tin (II) chloride (44908 mg, 237 mmol) in concentrated aqueous HCl (90 mL) was added at −10° C. The reaction mixture was stirred at room temperature for 8 h. The aqueous phase was treated with NaOH until a pH of 14 was achieved and then extracted with ethyl acetate (3×80 mL). The organic layers were combined and concentrated. The residue was purified by flash chromatography on silica gel (ethyl acetate in petroleum ether from 0% to 21%) to afford the title compound (10500 mg, 92% yield) as a grey solid.

LC-MS (Method 1): Retention time=1.39 min. MS (ESI) m/z 205/207 [M+H]⁺.

### Step 2. Synthesis of 1-(3-bromo-4-fluorophenyl)-3-(trifluoromethyl)-1,4,5,6-tetrahydro-7H-indazol-7-one (Intermediate D-3a)

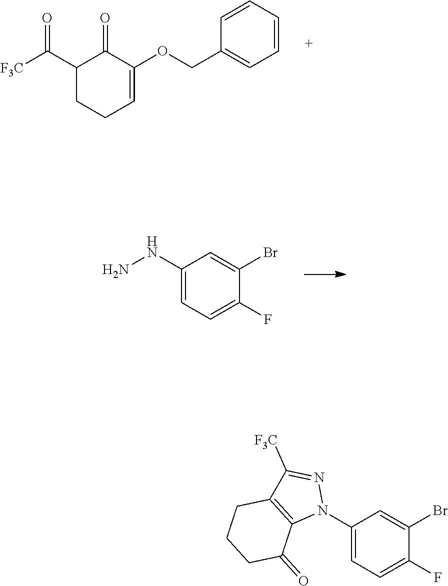 [SMILES: O=C1C(OCc2ccccc2)=CCCC1C(=O)C(F)(F)F ; NNc1ccc(F)c(Br)c1 ; O=C1CCCc2c(C(F)(F)F)nn(-c3ccc(F)c(Br)c3)c21]

To a solution of (3-bromo-4-fluoro-phenyl) hydrazine (6000 mg, 29.3 mmol) in ethanol (60 mL) was added 2-benzyloxy-6-(2,2,2-trifluoroacetyl)cyclohex-2-en-1-one (10474 mg, 35.1 mmol). The reaction was stirred at room temperature for 8 h. The reaction mixture was concentrated in vacuo to give a residue which was purified by flash chromatography on silica gel (ethyl acetate in petroleum ether from 0% to 20%) to afford the title compound (3500 mg, 31% yield) as a white solid.

LC-MS (Method 3): Retention time=2.23 min. MS (ESI) m/z 377/379 [M+H]⁺.

### Step 3. Synthesis of 1-(4-fluoro-3-hydroxyphenyl)-3-(trifluoromethyl)-1,4,5,6-tetrahydro-7H-indazol-7-one

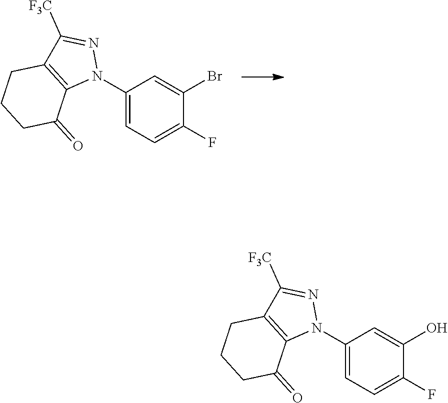 [SMILES: O=C1CCCc2c(C(F)(F)F)nn(-c3ccc(F)c(Br)c3)c21 ; O=C1CCCc2c(C(F)(F)F)nn(-c3ccc(F)c(O)c3)c21]

To a solution of 1-(3-bromo-4-fluoro-phenyl)-3-(trifluoromethyl)-5,6-dihydro-4H-indazol-7-one (Intermediate D-3a) (5000 mg, 13.3 mmol) in 1,4-dioxane (50 mL) and water (10 mL) were added 2-di-tert-butylphosphino-2′,4′,6′-triisopropylbiphenyl (563 mg, 1.33 mmol), cesium hydroxide monohydrate (6679 mg, 39.8 mmol), and Pd2 (dba) 3 (500 mg, 0.5 mmol). The reaction was stirred at 80° C. for 8 h. The reaction mixture was concentrated in vacuo to give a residue which was purified by prep-HPLC (NH₄HCO₃ as modifier) to afford the title compound (2000 mg, 47% yield) as a white solid.

LC-MS (Method 3): Retention time=1.98 min. MS (ESI) m/z 315.2 [M+H]⁺.

### Preparation of Intermediate D-4

### 1-(2-hydroxypyridin-4-yl)-3-(trifluoromethyl)-1,4,5,6-tetrahydro-7H-indazol-7-one

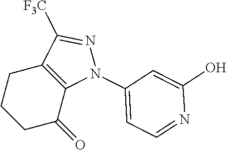 [SMILES: O=C1CCCc2c(C(F)(F)F)nn(-c3ccnc(O)c3)c21]

### Step 1. Synthesis of 4-bromo-2-(1-phenylethoxy)pyridine

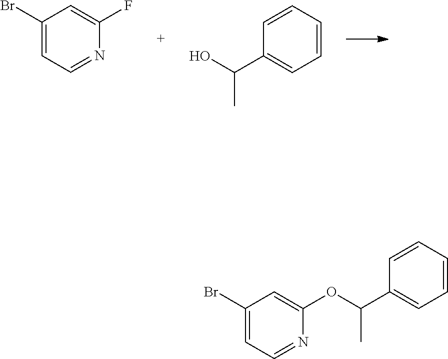

To the solution of 4-bromo-2-fluoro-pyridine (20000 mg, 114 mmol) and 1-phenylethanol (13908 mg, 114 mmol) in DMF (200 mL) was added cesium carbonate (111081 mg, 341 mmol). Then the mixture was stirred at 100° C. overnight. The crude product was then purified by flash chromatography eluted with 10% ethyl acetate in isohexane to get the title compound (29000 mg, 92% yield) as a yellow solid. LC-MS (Method 1): Retention time=2.45 min. MS (ESI) m/z 174.1/176.1 (M−PhCHMe+H)⁺.

### Step 2. Synthesis of tert-butyl 1-(2-(1-phenylethoxy)pyridine-4-yl) hydrazinecarboxylate

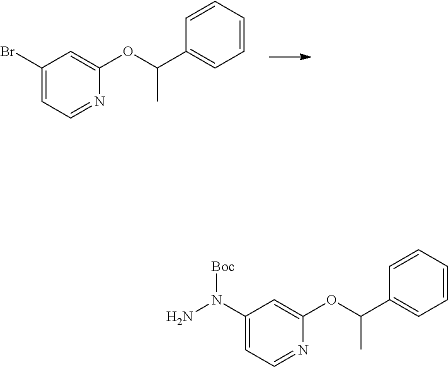 [SMILES: CC(Oc1cc(Br)ccn1)c1ccccc1 ; CC(Oc1cc(N(N)C(=O)OC(C)(C)C)ccn1)c1ccccc1]

To the solution of 4-bromo-2-(1-phenylethoxy)pyridine (15000 mg, 53.9 mmol) in 1,4-dioxane (150 mL) was added tert-butyl N-aminocarbamate (8553 mg, 64.7 mmol), (9,9-dimethyl-9H-xanthene-4,5-diyl)bis(diphenylphosphine) (3120 mg, 5.39 mmol), tris(dibenzylideneacetone)dipalladium (4938 mg, 5.39 mmol), and cesium carbonate (52714 mg, 162 mmol). Then the mixture was stirred at 100° C. overnight under Ar. The crude product was then purified by flash column chromatography eluted with 10% ethyl acetate in isohexane to get the title compound (16000 mg, 90% yield) as a yellow solid.

LC-MS (Method 1): Retention time=2.11 min. MS (ESI) m/z 330 [M+H]⁺.

### Step 3. Synthesis of 1-(2-hydroxypyridin-4-yl)-3-(trifluoromethyl)-1,4,5,6-tetrahydro-7H-indazol-7-one

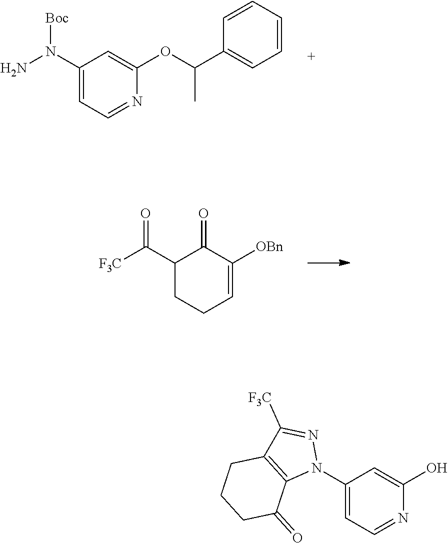 [SMILES: CC(Oc1cc(N(N)C(=O)OC(C)(C)C)ccn1)c1ccccc1 ; O=C1C(OCc2ccccc2)=CCCC1C(=O)C(F)(F)F ; O=C1CCCc2c(C(F)(F)F)nn(-c3ccnc(O)c3)c21]

To the solution of tert-butyl 1-(2-(1-phenylethoxy)pyridine-4-yl) hydrazinecarboxylate (10 g, 30.4 mmol) and 2-(benzyloxy)-6-(2,2,2-trifluoroacetyl)cyclohex-2-en-1-one in 2,2,2-trifluoroethanol (100 mL) was added sulfuric acid (25 mL). Then the mixture was stirred at 80° C. overnight. The mixture was poured into water (500 mL) and the pH was adjusted to 8-9 with NaHCO₃, then extracted with EA (3×50 mL). The organic layers were combined, washed with brine, dried over Na₂SO₄, filtered and concentrated. The crude product was then purified by flash column chromatography eluted with 10% ethyl acetate in isohexane to get the title compound (5 g, 55% yield) as a yellow solid.

LC-MS (Method 1): Retention time=1.72 min. MS (ESI) m/z 298.3 [M+H]⁺

### Preparation of Intermediate D-5

### 1-(5-hydroxy-3-pyridyl)-3-(trifluoromethyl)-5,6-dihydro-4H-indazol-7-one

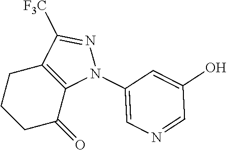 [SMILES: O=C1CCCc2c(C(F)(F)F)nn(-c3cncc(O)c3)c21]

### Step 1. Synthesis of tert-butyl 1-(5-bromopyridin-3-yl) hydrazine-1-carboxylate

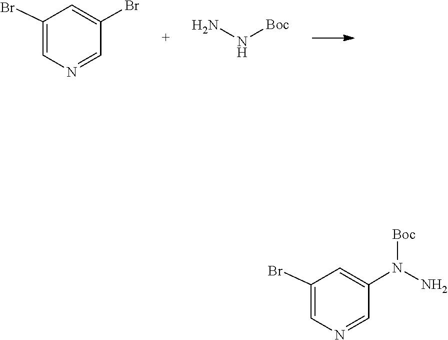 [SMILES: Brc1cncc(Br)c1 ; CC(C)(C)OC(=O)NN ; CC(C)(C)OC(=O)N(N)c1cncc(Br)c1]

To a solution of 3,5-dibromopyridine (10.0 g, 42.2 mmol) and tert-butyl N-aminocarbamate (5.58 g, 42.2 mmol) in 1,4-dioxane (100 mL) was added cesium carbonate (41.26 g, 127 mmol), (9,9-dimethyl-9h-xanthene-4,5-diyl)bis(diphenylphosphine) (2.44 g, 4.22 mmol) and tris(dibenzylideneacetone)dipalladium (3.87 g, 4.22 mmol). The reaction was stirred at 100° C. for 16 h. The reaction mixture was filtered through a Celite pad and the filtrate was concentrated. The residue was purified by flash chromatography (PE:EA=5:1) to give the title compound (4.10 g, 33% yield) as a yellow solid.

LC-MS (Method 3): Retention time=1.92 min. MS (ESI) m/z 288.1/290.1 [M+H]⁺.

¹H NMR (400 MHZ, DMSO-d6) δ 8.79 (s, 1H), 8.36 (d, J=1.9 Hz, 1H), 8.15 (t, J=1.9 Hz, 1H), 5.20 (s, 2H), 1.48 (s, 9H).

### Step 2. Synthesis of 1-(5-bromopyridin-3-yl)-3-(trifluoromethyl)-1,4,5,6-tetrahydro-7H-indazol-7-one

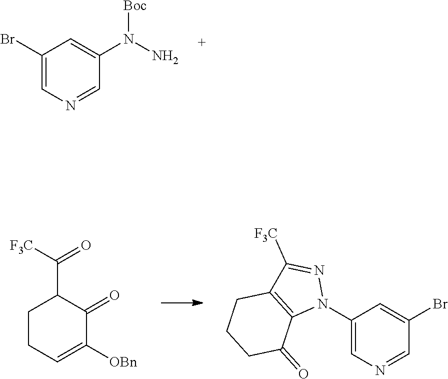 [SMILES: CC(C)(C)OC(=O)N(N)c1cncc(Br)c1 ; O=C1C(OCc2ccccc2)=CCCC1C(=O)C(F)(F)F ; O=C1CCCc2c(C(F)(F)F)nn(-c3cncc(Br)c3)c21]

To a solution of tert-butyl 1-(5-bromopyridin-3-yl) hydrazine-1-carboxylate (4.10 g, 14.2 mmol) and 2-benzyloxy-6-(2,2,2-trifluoroacetyl)cyclohex-2-en-1-one (4.67 g, 15.7 mmol) in 2,2,2-trifluoroethanol (20 mL, 265 mmol) was added sulfuric acid (5.0 mL, 93.8 mmol) dropwise at 0° C. The reaction was stirred at 80° C. for 6 h and cooled. To this solution was added 6N sodium hydroxide aqueous solution dropwise at 0° C. After addition, the mixture was extracted with ethyl acetate (3×50 mL). The combined organic layers were dried over Na₂SO₄, filtered and concentrated. The residue was purified by flash chromatography (PE:EA=5:1) to give the title compound (3.50 g, 65% yield) as a yellow solid.

LC-MS (Method 3): Retention time=2.11 min. MS (ESI) m/z 360.0/362.0 [M+H]⁺.

¹H NMR (400 MHZ, DMSO-d6) δ 8.87 (d, J=2.0 Hz, 1H), 8.81 (d, J=2.0 Hz, 1H), 8.44 (t, J=2.0 Hz, 1H), 2.91 (t, J=5.9 Hz, 2H), 2.65-2.58 (m, 2H), 2.20-2.12 (m, 2H).

### Step 3. Synthesis of 1-(5-hydroxy-3-pyridyl)-3-(trifluoromethyl)-5,6-dihydro-4H-indazol-7-one

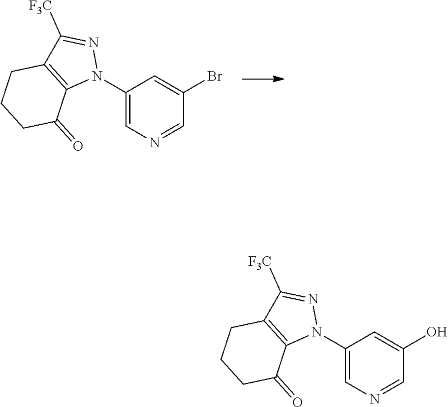 [SMILES: O=C1CCCc2c(C(F)(F)F)nn(-c3cncc(Br)c3)c21 ; O=C1CCCc2c(C(F)(F)F)nn(-c3cncc(O)c3)c21]

To a solution of 1-(5-bromopyridin-3-yl)-3-(trifluoromethyl)-1,4,5,6-tetrahydro-7H-indazol-7-one (3.50 g, 9.72 mmol) in 1,4-dioxane (15 mL) and water (5 mL) was added 2-di-tert-butylphosphino-2′,4′,6′-triisopropylbiphenyl (413 mg, 0.972 mmol), tris(dibenzylideneacetone) dipalladium (890 mg, 0.972 mmol) and cesium hydroxide monohydrate (4.90 g, 29.2 mmol). The reaction was stirred at 100° C. for 1 h. The reaction mixture was concentrated, and the residue was purified by reversed-phase flash chromatography (C18) (0.1% NH₄HCO₃ in water, 10-100% acetonitrile) to give the title compound (1.15 g, 40% yield) as an off-white solid.

LC-MS (Method 3): Retention time=1.81 min. MS (ESI) m/z 298.2 [M+H]⁺.

¹H NMR (500 MHZ, DMSO-d6) δ 10.45 (s, 1H), 8.26 (d, J=2.0 Hz, 1H), 8.22 (d, J=0.7 Hz, 1H), 7.36 (t, 1H), 2.90 (t, J=5.7 Hz, 2H), 2.63-2.57 (m, 2H), 2.19-2.11 (m, 2H).

### Preparation of Intermediate D-6

### 1-(6-chloropyridazin-4-yl)-3-(trifluoromethyl)-1,4,5,6-tetrahydro-7H-indazol-7-one

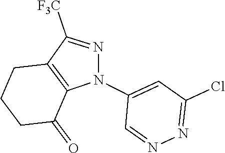 [SMILES: O=C1CCCc2c(C(F)(F)F)nn(-c3cnnc(Cl)c3)c21]

### Step 1. Synthesis of 1-(6-chloropyridazin-4-yl)-3-(trifluoromethyl)-1,4,5,6-tetrahydro-7H-indazol-7-one

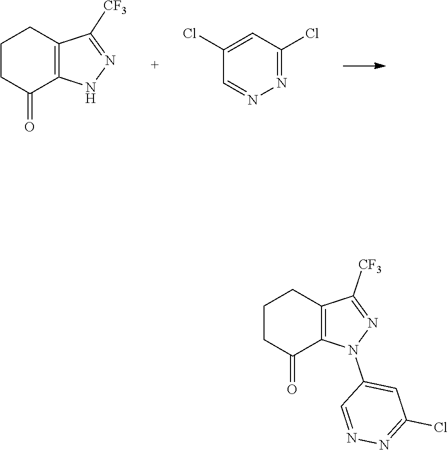 [SMILES: O=C1CCCc2c(C(F)(F)F)n[nH]c21 ; Clc1cnnc(Cl)c1 ; O=C1CCCc2c(C(F)(F)F)nn(-c3cnnc(Cl)c3)c21]

To the solution of 3,5-dichloropyridazine (1.82 g, 12.2 mmol) in DMF (30 mL) was added 3-(trifluoromethyl)-1,4,5,6-tetrahydroindazol-7-one (Intermediate D-1) (2.50 g, 12.2 mmol) and cesium carbonate (7980 mg, 24.5 mmol). The reaction was stirred at 80° C. for 2 h and then poured into water (200 mL). The solution was extracted with EA (3×50 mL). The combined organic layers were washed with brine (50 mL), dried over Na₂SO₄, filtered and concentrated. The crude was purified by flash chromatography (PE:EA=5:1) to get the title compound (1300 mg, 32% yield) as a yellow solid.

LC-MS (Method 2): Retention time=1.99 min. MS (ESI) m/z 317.1 [M+H]⁺.

### Preparation of Intermediate D-7

### 1-(4-hydroxypyridin-2-yl)-3-(trifluoromethyl)-1,4,5,6-tetrahydro-7H-indazol-7-one

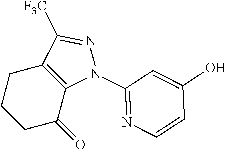 [SMILES: O=C1CCCc2c(C(F)(F)F)nn(-c3cc(O)ccn3)c21]

### Step 1. Synthesis of 1-(4-bromopyridin-2-yl)-3-(trifluoromethyl)-1,4,5,6-tetrahydro-7H-indazol-7-one (Intermediate D-7a)

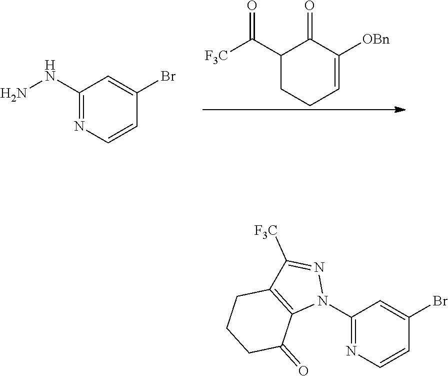 [SMILES: NNc1cc(Br)ccn1 ; O=C1C(OCc2ccccc2)=CCCC1C(=O)C(F)(F)F ; O=C1CCCc2c(C(F)(F)F)nn(-c3cc(Br)ccn3)c21]

To a solution of (4-bromo-2-pyridyl) hydrazine (630 mg, 3.35 mmol) and 2-benzyloxy-6-(2,2,2-trifluoroacetyl)cyclohex-2-en-1-one (1049 mg, 3.52 mmol) in TFE/H2SO4 (v/v=3:1) (10 mL). The reaction was stirred at 80° C. for 8 h. To the reaction mixture was added water (20 mL), the mixture was adjusted to pH=8 by addition of saturated aqueous sodium hydrogen carbonate. And then extracted with ethyl acetate (20 mL×3). The combined organic phase was washed with brine (40 mL), dried over anhydrous sodium sulfate, filtered, and concentrated in vacuo to give the residue, which was purified by flash silica gel column chromatography (ethyl acetate in petroleum ether from 0% to 25%) to afford the title compound (830 mg, 67.4% yield) as a yellow gum.

LCMS (Method 2): Retention time=1.95 min. MS (ESI) m/z 359.9 [M+H]⁺.

### Step 2. Synthesis of 1-(4-hydroxypyridin-2-yl)-3-(trifluoromethyl)-1,4,5,6-tetrahydro-7H-indazol-7-one

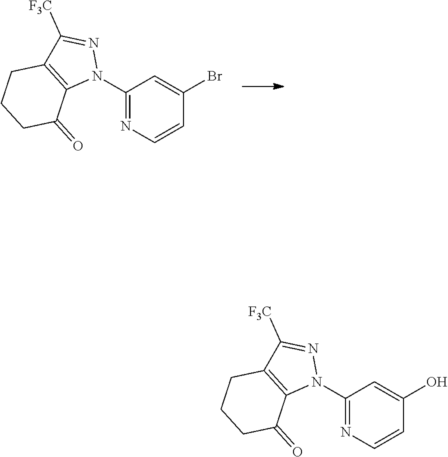 [SMILES: O=C1CCCc2c(C(F)(F)F)nn(-c3cc(Br)ccn3)c21 ; O=C1CCCc2c(C(F)(F)F)nn(-c3cc(O)ccn3)c21]

To a solution of 1-(4-bromo-2-pyridyl)-3-(trifluoromethyl)-5,6-dihydro-4H-indazol-7-one (830 mg, 2.30 mmol), cesium hydroxide monohydrate (1161 mg, 6.91 mmol), 2-di-tert-butylphosphino-2′,4′,6′-triisopropylbiphenyl (98 mg, 0.23 mmol) and tris(dibenzylideneacetone)dipalladium (106 mg, 0.12 mmol) in 1,4-dioxane (10 mL)/Water (2 mL) under N₂. The reaction was stirred at 80° C. for 8 h. The reaction mixture was added water (20 mL), and then extracted with ethyl acetate (20 mL×3). The combined organic phase was washed with brine (20 mL), dried over anhydrous sodium sulfate, filtered and concentrated in vacuum to give the residue, which was purified by prep-HPLC (NH₄HCO₃ condition) to afford the title compound (230 mg, 32.9% yield) as a grey solid.

LCMS (Method 2): Retention time=1.27 min. MS (ESI) m/z 298.0 [M+H]⁺.

### Preparation of Intermediate D-8

### 1-(6-hydroxypyridin-2-yl)-3-(trifluoromethyl)-1,4,5,6-tetrahydro-7H-indazol-7-one

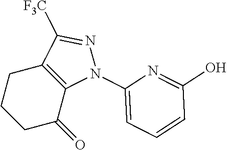 [SMILES: O=C1CCCc2c(C(F)(F)F)nn(-c3cccc(O)n3)c21]

### Preparation of Intermediate D-8a

### 1-(6-bromopyridin-2-yl)-3-(trifluoromethyl)-1,4,5,6-tetrahydro-7H-indazol-7-one

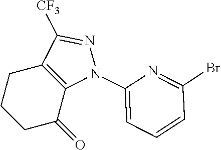 [SMILES: O=C1CCCc2c(C(F)(F)F)nn(-c3cccc(Br)n3)c21]

The Intermediate D-8 and Intermediate D-8a were prepared by following the procedures similar to Preparation of Intermediate D-7 and Intermediate D-7a.

Intermediate D-8: LCMS (Method 2): Retention time=1.50 min. MS (ESI) m/z 298.0 [M+H]⁺.

Intermediate D-8a: LCMS (Method 2): Retention time=1.96 min. MS (ESI) m/z 360.0 [M+H]⁺.

### Preparation of Intermediate D-9

### 1-(5-hydroxypyridazin-3-yl)-3-(trifluoromethyl)-1,4,5,6-tetrahydro-7H-indazol-7-one

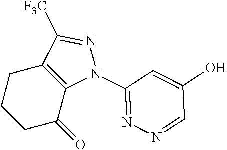 [SMILES: O=C1CCCc2c(C(F)(F)F)nn(-c3cc(O)cnn3)c21]

Intermediate D-9 was prepared essentially in the same manner as Intermediate D-4. (410 mg, 1.37 mmol, 26.4% yield) as a white solid.

LC-Mass (Method 1): Retention time=1.68 min. MS (ESI) m/z 299.1 [M+H]⁺.

¹H NMR (400 MHZ, MeOH-d4) δ 8.54 (d, J=2.6 Hz, 1H), 6.99 (d, J=2.6 Hz, 1H), 2.97 (t, J=6.0 Hz, 2H), 2.69-2.63 (m, 2H), 2.29-2.20 (m, 2H).

### Preparation of Intermediate D-10

### 1-(5-fluoro-4-hydroxypyridin-2-yl)-3-(trifluoromethyl)-1,4,5,6-tetrahydro-7H-indazol-7-one

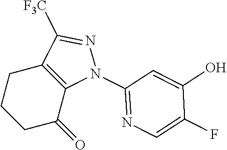 [SMILES: O=C1CCCc2c(C(F)(F)F)nn(-c3cc(O)c(F)cn3)c21]

### Step 1. Synthesis of 4-chloro-5-fluoro-2-hydrazineylpyridine

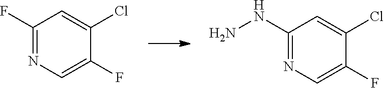 [SMILES: Fc1cc(Cl)c(F)cn1 ; NNc1cc(Cl)c(F)cn1]

A mixture of 4-chloro-2,5-difluoro-pyridine (1.00 eq, 4.00 g, 26.8 mmol) in IPA (15 mL) and hydrazine hydrate (2.00 eq, 2.6 mL, 53.5 mmol) was stirred at rt for 72 h. The mixture was concentrated under a vacuum. The residue was purified by reversed-phase flash chromatography (C18) (0.1% NH₄HCO₃ in water, 10-100% acetonitrile) to give 4-chloro-5-fluoro-2-pyridyl) hydrazine (1.00 g, 6.19 mmol, 23.1% yield) as a white solid.

LC-Mass (Method 2): Retention time=1.29 min. MS (ESI) m/z 162.1 [M+H]⁺.

### Step 2. Synthesis of 1-(4-chloro-5-fluoropyridin-2-yl)-3-(trifluoromethyl)-1,4,5,6-tetrahydro-7H-indazol-7-one

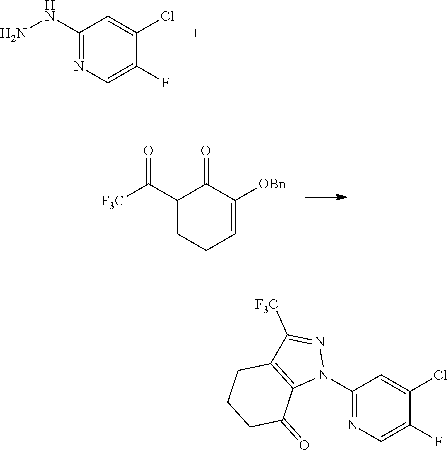 [SMILES: NNc1cc(Cl)c(F)cn1 ; O=C1C(OCc2ccccc2)=CCCC1C(=O)C(F)(F)F ; O=C1CCCc2c(C(F)(F)F)nn(-c3cc(Cl)c(F)cn3)c21]

To a solution of (4-chloro-5-fluoro-2-pyridyl) hydrazine (1.00 eq, 1.30 g, 8.05 mmol) and 2-benzyloxy-6-(2,2,2-trifluoroacetyl)cyclohex-2-en-1-one (1.10 eq, 2.64 g, 8.85 mmol) in 2,2,2-trifluoroethanol (32.9 eq, 20 mL, 265 mmol) was added sulfuric acid (11.7 eq, 5.0 mL, 93.8 mmol) dropwise at 0° C. The reaction was stirred at 80° C. for 6 h. To the reaction mixture was added sodium hydroxide aqueous solution (6N) dropwise at 0° C. The mixture was extracted with ethyl acetate (50 mL×3). The combined organic layers were dried over Na₂SO₄, filtered, and concentrated. The residue was purified by flash column chromatography (PE:EA=5:1) to give 1-(4-chloro-5-fluoro-2-pyridyl)-3-(trifluoromethyl)-5,6-dihydro-4H-indazol-7-one (1.30 g, 3.90 mmol, 48.4% yield) as a yellow solid.

LC-Mass (Method 1): Retention time=2.07 min. MS (ESI) m/z 334.0 [M+H]⁺.

### Step 3. Synthesis of 1-(5-fluoro-4-hydroxypyridin-2-yl)-3-(trifluoromethyl)-1,4,5,6-tetrahydro-7H-indazol-7-one

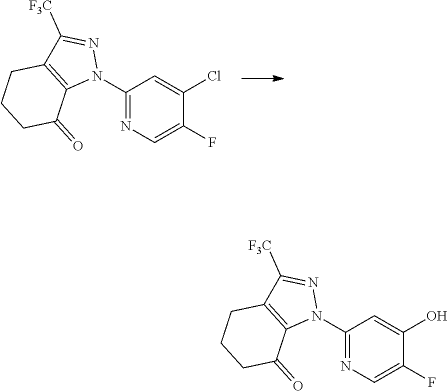 [SMILES: O=C1CCCc2c(C(F)(F)F)nn(-c3cc(Cl)c(F)cn3)c21 ; O=C1CCCc2c(C(F)(F)F)nn(-c3cc(O)c(F)cn3)c21]

To a solution of 1-(4-chloro-5-fluoro-2-pyridyl)-3-(trifluoromethyl)-5,6-dihydro-4H-indazol-7-one (1.00 eq, 90 mg, 0.270 mmol) in 1,4-dioxane (15 mL) and water (5.00 eq, 24 mg, 1.35 mmol) was added cesium carbonate (3.00 eq, 264 mg, 0.809 mmol) and RockPhos Pd G3 (CAS #: 2009020-38-4) (0.10 eq, 23 mg, 0.0270 mmol) ( ). The reaction mixture was stirred at 100° C. for 30 min under nitrogen. The mixture was concentrated and purified by flash column chromatography (DCM:MeOH=10:1) to give the crude product. The crude product was further purified by reversed-phase flash chromatography (C18) (0.1% NH₄HCO₃ in water, 10-100% acetonitrile) to give 1-(5-fluoro-4-hydroxy-2-pyridyl)-3-(trifluoromethyl)-5,6-dihydro-4H-indazol-7-one (20 mg, 0.0634 mmol, 23.5% yield) as a white solid.

LC-Mass (Method 1): Retention time=1.82 min. MS (ESI) m/z 316.1 [M+H]⁺.

### Preparation of Intermediate D-11

### 3-iodo-1,5,6,7-tetrahydroindazol-4-one

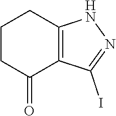 [SMILES: O=C1CCCc2[nH]nc(I)c21]

### Step 1. Synthesis of 2-(dimethylaminomethylene)cyclohexane-1,3-dione

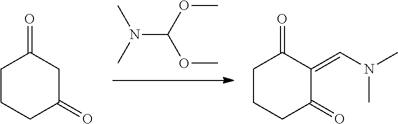

To a solution of cyclohexane-1,3-dione (18.0 g, 161.00 mmol) in DMF-DMA (45 mL, 336.00 mmol) was stirred at 80° C. for 2 h. The mixture was concentrated to give a residue which was washed with petroleum ether (100 mL×2) and filtered to obtain 2-(dimethylaminomethylene)cyclohexane-1,3-dione (24.0 g, 84.9% yield) as a yellow solid.

LCMS (Method 1): Retention time=1.11 min. MS (ESI) m/z 168.2 [M+H]⁺.

### Step 2. Synthesis of 1,5,6,7-tetrahydroindazol-4-one

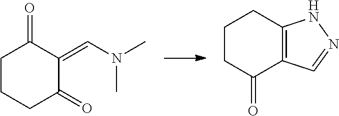

To a solution of 2-(dimethylaminomethylene)cyclohexane-1,3-dione (24.0 g, 144.00 mmol) in ethanol (300 mL) was added acetic acid (21 mL) and hydrazine hydrate (3588 mg, 71.80 mmol). The reaction was stirred at 80° C. for 16 h. The mixture was concentrated to give a residue which was purified by flash column chromatography (petroleum ether/ethyl acetate=5/1) to obtain 1,5,6,7-tetrahydroindazol-4-one (8.0 g, 40.9%) as a yellow solid.

LCMS (Method 1): Retention time=1.12 min. MS (ESI) m/z 137.1 [M+H]⁺.

### Step 3. Synthesis of 3-iodo-1,5,6,7-tetrahydroindazol-4-one

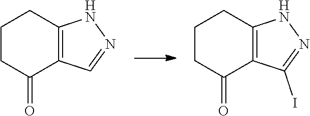 [SMILES: O=C1CCCc2[nH]ncc21 ; O=C1CCCc2[nH]nc(I)c21]

To a solution of 1,5,6,7-tetrahydroindazol-4-one (8.0 g, 58.80 mmol) in DMF (200 mL) was added n-iodosuccinimide (16.0 g, 71.10 mmol). The reaction was stirred at 80° C. for 8 h. The mixture was washed with water (1000 mL) and extracted with ethyl acetate (300 mL×3). The combined organic phase was concentrated to give a residue which was purified by flash column chromatography (petroleum ether/ethyl acetate=2/1) to obtain 3-iodo-1,5,6,7-tetrahydroindazol-4-one (3000 mg, 18.5%) as a light-yellow solid.

LCMS (Method 1): Retention time=1.44 min. MS (ESI) m/z 263.0 [M+H]⁺.

### Preparation of the Intermediate E1

### methyl 4-((1-(2-hydroxypyridin-4-yl)-3-(trifluoromethyl)-4,5,6,7-tetrahydro-1H-pyrazolo[4,3-c]pyridine-7-yl)oxy)benzoate

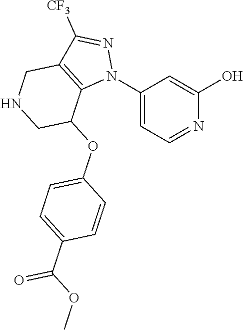 [SMILES: COC(=O)c1ccc(OC2CNCc3c(C(F)(F)F)nn(-c4ccnc(O)c4)c32)cc1]

### Step 1. Synthesis of tert-butyl 3-bromo-4-oxo-piperidine-1-carboxylate

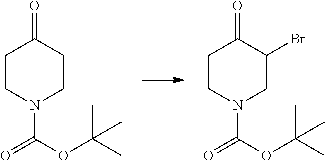

To a stirred solution of 1-boc-4-piperidone (1.00 eq, 200 mg, 1.00 mmol) in chloroform (2 mL) was slowly added bromine (1.10 eq, 0.057 mL, 1.10 mmol) under N₂ at 0° C. Stirring was continued at 0° C. for 15 min then slowly warmed up to rt and stirred for 16 h. TLC showed one nonpolar spot. The sample was used in the next step of the reaction without purification and characterization.

### Step 2. Synthesis of tert-butyl 3-(4-(methoxycarbonyl)phenoxy)-4-oxopiperidine-1-carboxylate

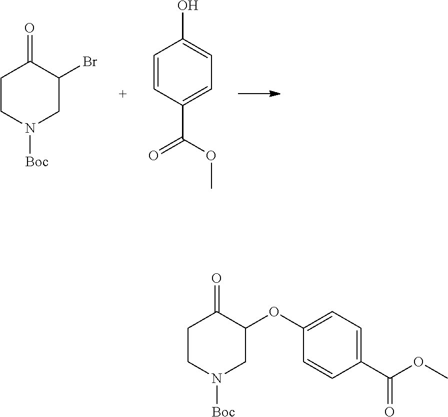 [SMILES: CC(C)(C)OC(=O)N1CCC(=O)C(Br)C1 ; COC(=O)c1ccc(O)cc1 ; COC(=O)c1ccc(OC2CN(C(=O)OC(C)(C)C)CCC2=O)cc1]

To a solution of tert-butyl 3-bromo-4-oxo-piperidine-1-carboxylate (5000 mg, 18.00 mmol), methyl 4-hydroxybenzoate (3009 mg, 19.8 mmol) in acetonitrile (50 mL) was added potassium carbonate (4969 mg, 36.00 mmol). The reaction was stirred at 85° C. for 8 h. The reaction mixture was concentrated in vacuo to give the title compound (5800 mg, 78.5% yield) as a yellow solid.

LCMS (Method 2): Retention time=1.88 min. MS (ESI) m/z 294.1 [M+H−tBu]⁺.

### Step 3. Synthesis of tert-butyl 3-(4-(methoxycarbonyl)phenoxy)-4-oxo-5-(2,2,2-trifluoroacetyl)piperidine-1-carboxylate

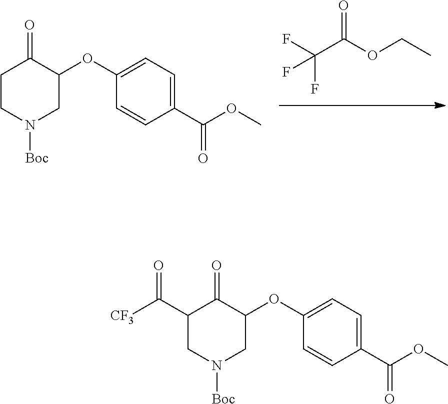

To a solution of tert-butyl 3-(4-methoxycarbonylphenoxy)-4-oxo-piperidine-1-carboxylate (4100 mg, 11.70 mmol) and ethyl trifluoroacetate (2.8 mL, 23.50 mmol) in THF (45 mL) was added lithium diisopropylamide (2514 mg, 23.50 mmol) at −78° C. under N₂. The reaction was stirred at room temperature for 4 h. The reaction mixture was concentrated in vacuum to give the title compound (5000 mg, 76.5% yield) as a yellow solid.

LCMS (Method 2): Retention time=1.51 min. MS (ESI) m/z 446.0 [M+H]⁺.

### Step 4. Synthesis of tert-butyl 1-(2-hydroxypyridin-4-yl)-7-(4-(methoxycarbonyl)phenoxy)-3-(trifluoromethyl)-1,4,6,7-tetrahydro-5H-pyrazolo[4,3-c]pyridine-5-carboxylate

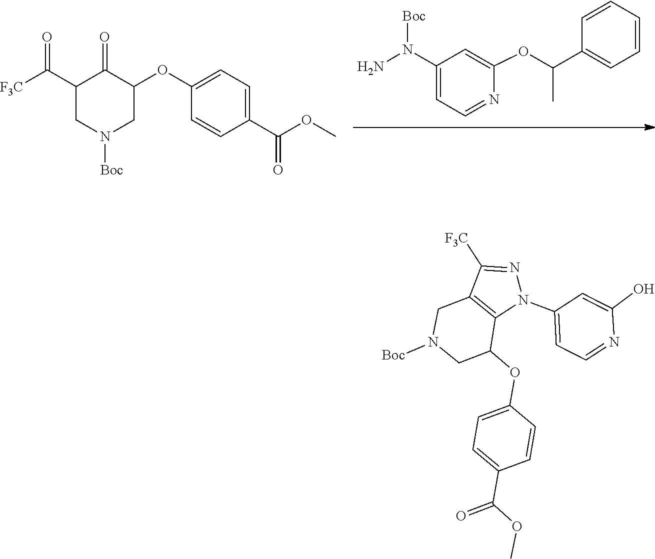 [SMILES: COC(=O)c1ccc(OC2CN(C(=O)OC(C)(C)C)CC(C(=O)C(F)(F)F)C2=O)cc1 ; CC(Oc1cc(N(N)C(=O)OC(C)(C)C)ccn1)c1ccccc1 ; COC(=O)c1ccc(OC2CN(C(=O)OC(C)(C)C)Cc3c(C(F)(F)F)nn(-c4ccnc(O)c4)c32)cc1]

To a solution of tert-butyl 3-(4-methoxycarbonylphenoxy)-4-oxo-5-(2,2,2-trifluoroacetyl)piperidine-1-carboxylate (5000 mg, 11.20 mmol) in acetic acid (50 mL) were added tert-butyl N-amino-N-[2-(1-phenylethoxy)-4-pyridyl]carbamate (4068 mg, 12.30 mmol) and (2,4-bis(4-methoxyphenyl)-1,3-dithia-2,4-diphosphetane-2,4-disulfide) (9081 mg, 22.50 mmol). The reaction was stirred 80° C. for 8 h. The reaction mixture was concentrated in vacuo to give a residue, which was purified by flash silica gel column chromatography (ethyl acetate in petroleum ether from 0% to 100%) to afford the title compound (2700 mg, 36.0% yield) as a yellow solid.

LCMS (Method 1): Retention time=1.97 min. MS (ESI) m/z 535.0 [M+H]⁺.

### Step 5. Synthesis of methyl 4-((1-(2-hydroxypyridin-4-yl)-3-(trifluoromethyl)-4,5,6,7-tetrahydro-1H-pyrazolo[4,3-c]pyridine-7-yl)oxy)benzoate

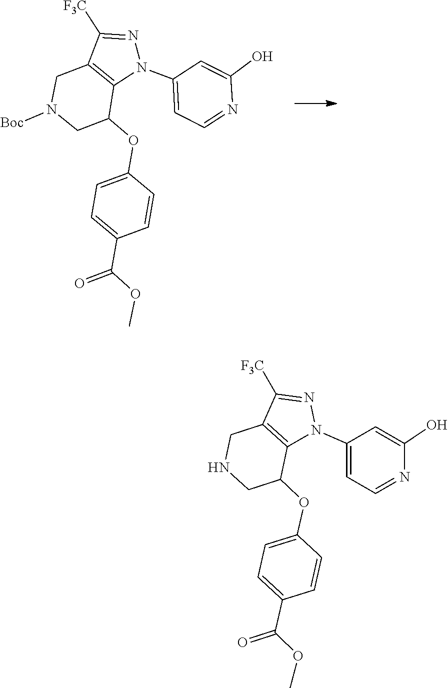 [SMILES: COC(=O)c1ccc(OC2CN(C(=O)OC(C)(C)C)Cc3c(C(F)(F)F)nn(-c4ccnc(O)c4)c32)cc1 ; COC(=O)c1ccc(OC2CNCc3c(C(F)(F)F)nn(-c4ccnc(O)c4)c32)cc1]

Tert-butyl 1-(2-hydroxy-4-pyridyl)-7-(4-methoxycarbonylphenoxy)-3-(trifluoromethyl)-6,7-dihydro-4H-pyrazolo[4,3-c]pyridine-5-carboxylate (2700 mg, 5.05 mmol) was dissolved in HCl in 1,4-dioxane (4M, 30 mL). The reaction was stirred at room temperature for 1 h. The reaction mixture was concentrated in vacuo to give a residue which was purified by flash silica gel column chromatography (methanol in dichloromethane from 0% to 7%) to afford the title compound (650 mg, 23.7% yield) as a yellow solid.

LCMS (Method 2): Retention time=1.46 min. MS (ESI) m/z 435.1 [M+H]⁺.

### Examples 1 and 2

### (R)-3-((1-(6-(1-(2,2-difluoro-[1,3]dioxolo[4,5-c]pyridin-6-yl)ethoxy)pyridazin-4-yl)-3-(trifluoromethyl)-1,4,5,6-tetrahydro-7H-pyrazolo[3,4-b]pyridin-7-yl)methyl)bicyclo[1.1.1]pentane-1-carboxylic acid

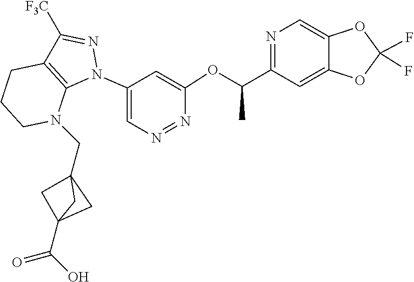 [SMILES: C[C@@H](Oc1cc(-n2nc(C(F)(F)F)c3c2N(CC24CC(C(=O)O)(C2)C4)CCC3)cnn1)c1cc2c(cn1)OC(F)(F)O2]

### (S)-3-((1-(6-(1-(2,2-difluoro-[1,3]dioxolo[4,5-c]pyridin-6-yl)ethoxy)pyridazin-4-yl)-3-(trifluoromethyl)-1,4,5,6-tetrahydro-7H-pyrazolo[3,4-b]pyridin-7-yl)methyl)bicyclo[1.1.1]pentane-1-carboxylic acid

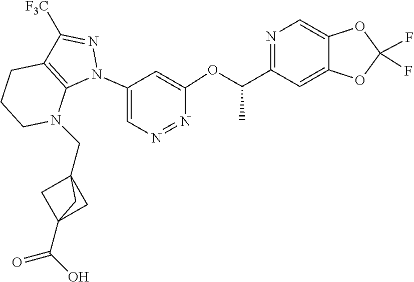 [SMILES: C[C@H](Oc1cc(-n2nc(C(F)(F)F)c3c2N(CC24CC(C(=O)O)(C2)C4)CCC3)cnn1)c1cc2c(cn1)OC(F)(F)O2]

### Step 1. Synthesis of methyl 3-((1-(6-chloropyridazin-4-yl)-3-(trifluoromethyl)-5,6-dihydro-1H-pyrazolo[3,4-b]pyridine-7 (4H)-yl)methyl)bicyclo[1.1.1]pentane-1-carboxylate

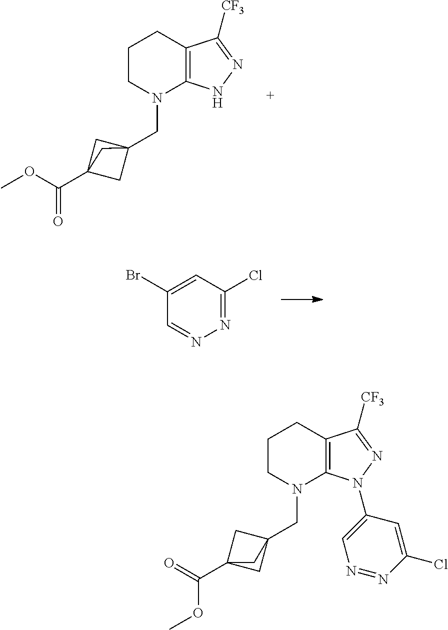 [SMILES: COC(=O)C12CC(CN3CCCc4c(C(F)(F)F)n[nH]c43)(C1)C2 ; Clc1cc(Br)cnn1 ; COC(=O)C12CC(CN3CCCc4c(C(F)(F)F)nn(-c5cnnc(Cl)c5)c43)(C1)C2]

To the solution of methyl 3-[[3-(trifluoromethyl)-1,4,5,6-tetrahydropyrazolo[3,4-b]pyridine-7-yl]methyl]bicyclo[1.1.1]pentane-1-carboxylate (Intermediate A-1) (370 mg, 1.12 mmol) in DMF (5 mL) was added 5-bromo-3-chloro-pyridazine (261 mg, 1.35 mmol) and Cs₂CO₃ (549 mg, 1.69 mmol). The reaction was stirred at 70° C. for 2 h. The reaction mixture was poured into water (50 mL) and extracted with EA (3×30 mL). The organic layers were combined and washed with brine, dried over Na₂SO₄, filtered, and concentrated. The crude product was purified by flash chromatography (10-20% EA in heptane) to get the title compound (50 mg, 10% yield) as a white solid.

LC-MS (Method 3): Retention time=2.30 min. MS (ESI) m/z 442.2 [M+H]⁺.

### Step 2. Synthesis of (R)-3-((1-(6-(1-(2,2-difluoro-[1,3]dioxolo[4,5-c]pyridin-6-yl)ethoxy)pyridazin-4-yl)-3-(trifluoromethyl)-1,4,5,6-tetrahydro-7H-pyrazolo[3,4-b]pyridin-7-yl)methyl)bicyclo[1.1.1]pentane-1-carboxylic acid and(S)-3-((1-(6-(1-(2,2-difluoro-[1,3]dioxolo[4,5-c]pyridin-6-yl)ethoxy)pyridazin-4-yl)-3-(trifluoromethyl)-1,4,5,6-tetrahydro-7H-pyrazolo[3,4-b]pyridin-7-yl)methyl)bicyclo[1.1.1]pentane-1-carboxylic acid

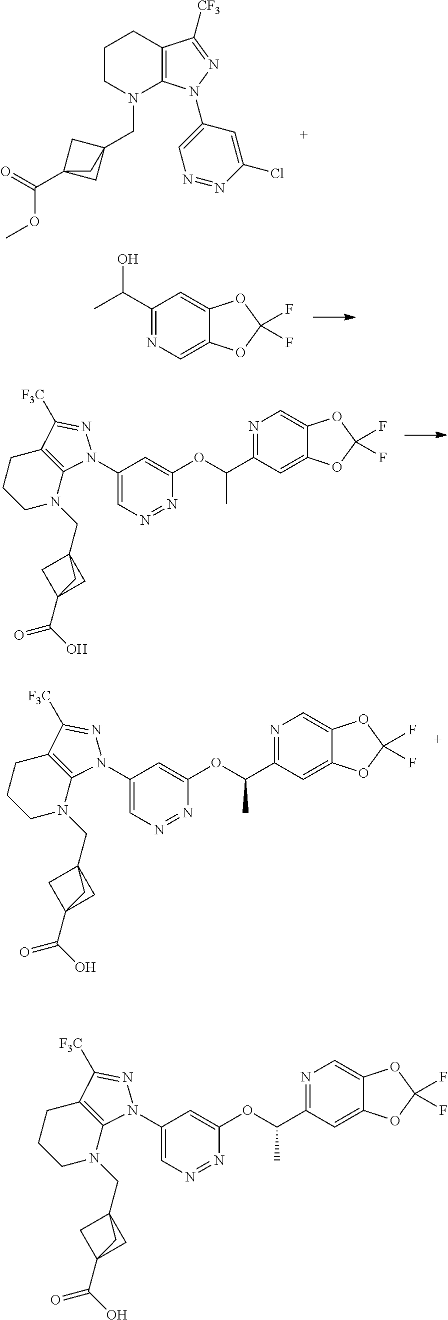 [SMILES: COC(=O)C12CC(CN3CCCc4c(C(F)(F)F)nn(-c5cnnc(Cl)c5)c43)(C1)C2 ; CC(O)c1cc2c(cn1)OC(F)(F)O2 ; CC(Oc1cc(-n2nc(C(F)(F)F)c3c2N(CC24CC(C(=O)O)(C2)C4)CCC3)cnn1)c1cc2c(cn1)OC(F)(F)O2 ; C[C@@H](Oc1cc(-n2nc(C(F)(F)F)c3c2N(CC24CC(C(=O)O)(C2)C4)CCC3)cnn1)c1cc2c(cn1)OC(F)(F)O2 ; C[C@H](Oc1cc(-n2nc(C(F)(F)F)c3c2N(CC24CC(C(=O)O)(C2)C4)CCC3)cnn1)c1cc2c(cn1)OC(F)(F)O2]

To the solution of 1-(2,2-difluoro-[1,3]dioxolo[4,5-c]pyridine-6-yl)ethanol (Intermediate C-2) (46 mg, 0.226 mmol) in THF (3 mL) was added NaH (9.1 mg, 0.226 mmol) at 0° C. and stirred at rt for 30 min. Then methyl 3-[[1-(6-chloropyridazin-4-yl)-3-(trifluoromethyl)-5,6-dihydro-4H-pyrazolo[3,4-b]pyridine-7-yl]methyl]bicyclo[1.1.1]pentane-1-carboxylate (50 mg, 0.113 mmol) was added. The reaction solution was stirred at rt for 1 h. The solution was concentrated and purified by Prep-HPLC to get 18 mg of the product as a white solid. This enantiomeric mixture was separated by SFC to get the title compound with the first fraction designated as P1 (7.2 mg, 11% yield, a white solid) and the second fraction as P2 (7.8 mg, 12% yield, a white solid).

Chiral HPLC: Column: OJ-3 4.6×100 mm 3 μm; Cosolvent: MeOH [0.2% NH₃ (7M in MeOH)].

P1: retention time=0.940 min

LC-MS (Method 2): Retention time=1.77 min. MS (ESI) m/z 595.2 [M+H]⁺.

¹H NMR (500 MHZ, CH₃OH-d₄) δ 9.32 (d, J=2.0 Hz, 1H), 8.40 (s, 1H), 7.70 (d, J=2.0 Hz, 1H), 7.56 (s, 1H), 6.39 (q, J=6.6 Hz, 1H), 3.30-3.25 (m, 2H), 2.92 (s, 2H), 2.64 (t, J=6.3 Hz, 2H), 2.03-1.95 (m, 6H), 1.86-1.79 (m, 2H), 1.76 (d, J=6.6 Hz, 3H).

P2: retention time=2.196 min.

LC-MS (Method 2): Retention time=1.77 min. MS (ESI) m/z 595.2 [M+H]⁺.

¹H NMR (500 MHZ, CH₃OH-d₄ δ 9.32 (d, J=2.0 Hz, 1H), 8.40 (s, 1H), 7.70 (d, J=2.0 Hz, 1H), 7.56 (s, 1H), 6.39 (q, J=6.6 Hz, 1H), 3.30-3.25 (m, 2H), 2.92 (s, 2H), 2.64 (t, J=6.3 Hz, 2H), 2.03-1.95 (m, 6H), 1.86-1.79 (m, 2H), 1.76 (d, J=6.6 Hz, 3H).

### Examples 3 and 4

### (R)-4-((1-(6-(1-(2,2-difluorobenzo[d][1,3]dioxol-5-yl)ethoxy)pyridazin-4-yl)-3-(trifluoromethyl)-1,4,5,6-tetrahydro-7H-pyrazolo[3,4-b]pyridin-7-yl)methyl)benzoic acid

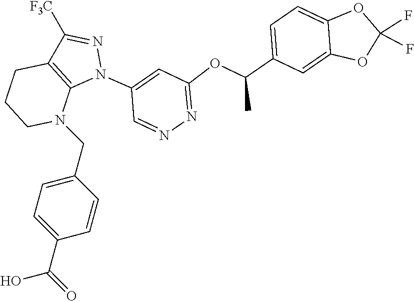 [SMILES: C[C@@H](Oc1cc(-n2nc(C(F)(F)F)c3c2N(Cc2ccc(C(=O)O)cc2)CCC3)cnn1)c1ccc2c(c1)OC(F)(F)O2]

### (S)-4-((1-(6-(1-(2,2-difluorobenzo[d][1,3]dioxol-5-yl)ethoxy)pyridazin-4-yl)-3-(trifluoromethyl)-1,4,5,6-tetrahydro-7H-pyrazolo[3,4-b]pyridin-7-yl)methyl)benzoic acid

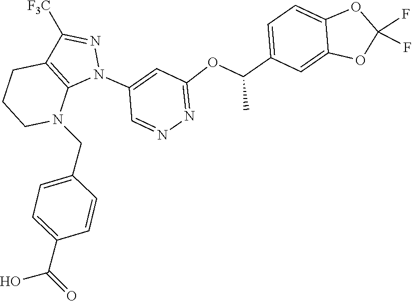 [SMILES: C[C@H](Oc1cc(-n2nc(C(F)(F)F)c3c2N(Cc2ccc(C(=O)O)cc2)CCC3)cnn1)c1ccc2c(c1)OC(F)(F)O2]

Examples 3 and 4 were synthesized following the procedures for the synthesis of Example 1 and 2.

Chiral Prep conditions: Column: IG 20×250 mm, 10 μm; Mobile phase: CO₂/MeOH (0.2% Ammonia in Methanol)=65/35.

P1: Retention time=1.51 min

LC-MS (Method 2): Retention time=1.86 min. MS (ESI) m/z 604 [M+H]⁺.

¹H NMR (500 MHZ, CH₃OH-d₄) δ 9.28 (d, J=2.0 Hz, 1H), 8.05 (d, J=8.0 Hz, 2H), 7.66 (d, J=2.0 Hz, 1H), 7.45 (d, J=7.9 Hz, 2H), 7.12-6.99 (m, 3H), 6.24 (q, J=6.4 Hz, 1H), 4.11 (s, 2H), 3.29-3.25 (m, 2H), 2.74-2.62 (m, 2H), 1.87-1.72 (m, 2H), 1.56 (d, J=6.0 Hz, 3H).

P2: Retention time=2.21 min

LC-MS (Method 2): Retention time=1.86 min. MS (ESI) m/z 604 [M+H]⁺.

¹H NMR (500 MHZ, CH₃OH-d₄) δ 9.28 (d, J=2.0 Hz, 1H), 8.05 (d, J=8.0 Hz, 2H), 7.66 (d, J=2.0 Hz, 1H), 7.45 (d, J=7.9 Hz, 2H), 7.12-6.99 (m, 3H), 6.24 (q, J=6.4 Hz, 1H), 4.10 (s, 2H), 3.29-3.25 (m, 2H), 2.74-2.62 (m, 2H), 1.87-1.72 (m, 2H), 1.56 (d, J=6.0 Hz, 3H).

### Examples 5 and 6

### (R)-3-((1-(6-(1-(2,2-difluorobenzo[d][1,3]dioxol-5-yl)ethoxy)pyridazin-4-yl)-3-(trifluoromethyl)-1,4,5,6-tetrahydro-7H-pyrazolo[3,4-b]pyridin-7-yl)methyl)bicyclo[1.1.1]pentane-1-carboxylic acid

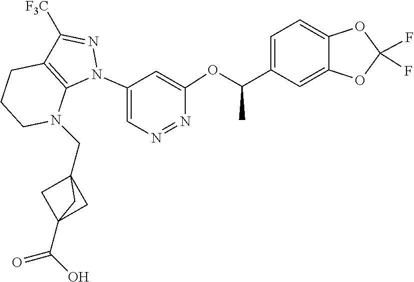 [SMILES: C[C@@H](Oc1cc(-n2nc(C(F)(F)F)c3c2N(CC24CC(C(=O)O)(C2)C4)CCC3)cnn1)c1ccc2c(c1)OC(F)(F)O2]

### (S)-3-((1-(6-(1-(2,2-difluorobenzo[d][1,3]dioxol-5-yl)ethoxy)pyridazin-4-yl)-3-(trifluoromethyl)-1,4,5,6-tetrahydro-7H-pyrazolo[3,4-b]pyridin-7-yl)methyl)bicyclo[1.1.1]pentane-1-carboxylic acid

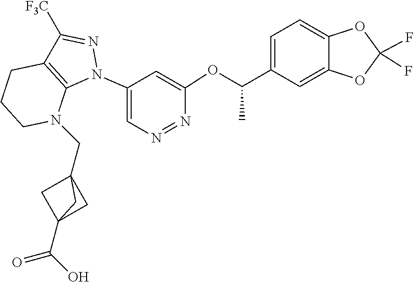 [SMILES: C[C@H](Oc1cc(-n2nc(C(F)(F)F)c3c2N(CC24CC(C(=O)O)(C2)C4)CCC3)cnn1)c1ccc2c(c1)OC(F)(F)O2]

Examples 5 and 6 were synthesized by following the procedures for the synthesis of Example 1 and 2.

P1. Retention time=0.991 min. (SFC Method: (R,R) Whelk-O1 4.6×100 mm 3.5 μm).

LC-MS (Method 2): Retention time=1.84 min. MS (ESI) m/z 594.1 [M+H]⁺.

¹H NMR (500 MHZ, CH₃OH-d₄) δ 9.29 (d, J=1.8 Hz, 1H), 7.64 (d, J=1.9 Hz, 1H), 7.40 (d, J=1.3 Hz, 1H), 7.35 (d, J=8.3 Hz, 1H), 7.17 (d, J=8.3 Hz, 1H), 6.42 (q, J=6.5 Hz, 1H), 3.29-3.23 (m, 2H), 2.89 (s, 2H), 2.63 (t, J=6.3 Hz, 2H), 1.97 (s, 6H), 1.84-1.78 (m, 2H), 1.73 (d, J=6.5 Hz, 3H).

P2: Retention time=1.556 min. (SFC Method: (R,R) Whelk-O1 4.6×100 mm 3.5 μm).

LC-MS (Method 2): Retention time=1.84 min. MS (ESI) m/z 594.1 [M+H]⁺.

¹H NMR (500 MHz, CH₃OH-d₄) δ 9.29 (d, J=1.8 Hz, 1H), 7.64 (d, J=1.8 Hz, 1H), 7.40 (d, J=1.8 Hz, 1H), 7.35 (dd, J=8.3 Hz, 1.8 Hz, 1H), 7.17 (d, J=8.3 Hz, 1H), 6.42 (q, J=6.5 Hz, 1H), 3.29-3.23 (m, 2H), 2.89 (s, 2H), 2.63 (t, J=6.3 Hz, 2H), 1.97 (s, 6H), 1.84-1.78 (m, 2H), 1.73 (d, J=6.5 Hz, 3H).

### Example 7

### 4-((1-(2-((S)-1-(2,2-difluorobenzo[d][1,3]dioxol-5-yl)ethoxy)pyridine-4-yl)-3-(trifluoromethyl)-1,4,5,6-tetrahydro-7H-pyrazolo[3,4-b]pyridine-7-yl)methyl)cyclohexane-1-carboxylic acid

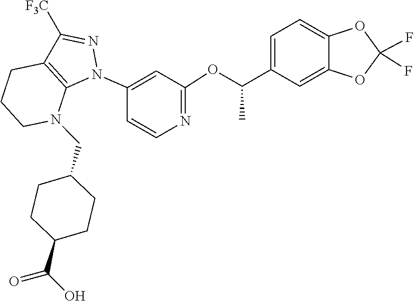 [SMILES: C[C@H](Oc1cc(-n2nc(C(F)(F)F)c3c2N(C[C@H]2CC[C@H](C(=O)O)CC2)CCC3)ccn1)c1ccc2c(c1)OC(F)(F)O2]

Example 7 was synthesized by following the procedures for the synthesis of Example 1 and 2.

Chiral Prep conditions: Column: AD-3 4.6×100 mm, 3 μm. Retention time=1.86 min.

LC-MS (Method 2): Retention time=1.87 min. MS (ESI) m/z 609.3 [M+H]⁺.

¹H NMR (400 MHZ, CH₃OH-d₄) δ 8.18 (d, J=5.2 Hz, 1H), 7.32 (s, 1H), 7.24-7.27 (m, 2H), 7.20 (s, 1H), 7.14 (d, J=8.0 Hz, 1H), 6.24 (q, J=6.0 Hz, 1H), 3.24 (d, J=4.4 Hz, 2H), 2.62 (t, J=6.4 Hz, 2H), 2.55 (d, J=7.2 Hz, 2H), 1.58-2.04 (m, 11H), 1.28-1.37 (m, 2H), 0.66-0.71 (m, 2H).

### Example 8

### 4-[[1-[2-[(1S)-1-(2,2-difluoro-1,3-benzodioxol-5-yl)ethoxy]-4-pyridyl]-3-(trifluoromethyl)-5,6-dihydro-4H-pyrazolo[3,4-b]pyridine-7-yl]methyl]benzoic acid

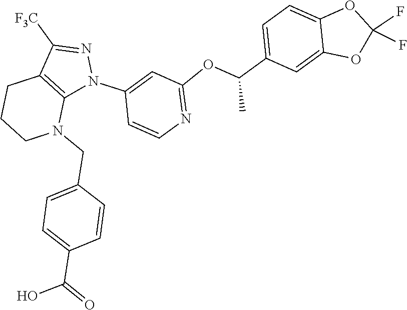 [SMILES: C[C@H](Oc1cc(-n2nc(C(F)(F)F)c3c2N(Cc2ccc(C(=O)O)cc2)CCC3)ccn1)c1ccc2c(c1)OC(F)(F)O2]

### Step 1. Synthesis of methyl 4-((1-(2-fluoropyridin-4-yl)-3-(trifluoromethyl)-5,6-dihydro-1H-pyrazolo[3,4-b]pyridine-7 (4H)-yl)methyl)benzoate

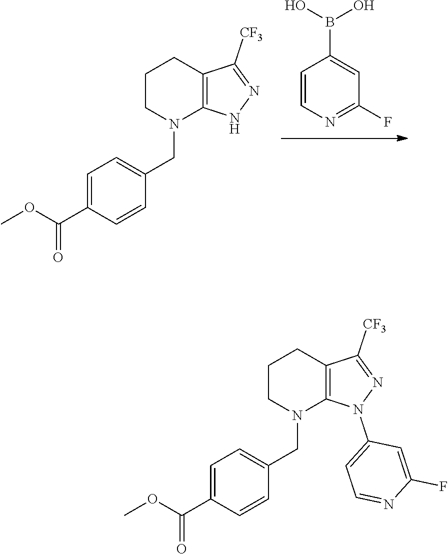 [SMILES: COC(=O)c1ccc(CN2CCCc3c(C(F)(F)F)n[nH]c32)cc1 ; OB(O)c1ccnc(F)c1 ; COC(=O)c1ccc(CN2CCCc3c(C(F)(F)F)nn(-c4ccnc(F)c4)c32)cc1]

To the solution of methyl 4-[[3-(trifluoromethyl)-1,4,5,6-tetrahydropyrazolo[3,4-b]pyridine-7-yl]methyl]benzoate (Intermediate A-2) (1.00 g, 2.95 mmol) in DCM (20 mL) was added (2-fluoro-4-pyridyl) boronic acid (831 mg, 5.89 mmol), copper (II) acetate (268 mg, 1.47 mmol) and TEA (0.41 mL, 2.95 mmol). The solution was stirred at rt for 3 days. The solution was concentrated, and the crude product was purified by flash chromatography eluted with 15% EA in PE to get the title compound (100 mg, 8% yield) as a white solid.

LC-MS (Method 2): Retention time=1.88 min. MS (ESI) m/z 435 [M+H]⁺.

### Step 2. Synthesis of(S)-4-((1-(2-(1-(2,2-difluorobenzo[d][1,3]dioxol-5-yl)ethoxy)pyridine-4-yl)-3-(trifluoromethyl)-5,6-dihydro-1H-pyrazolo[3,4-b]pyridine-7 (4H)-yl)methyl)benzoic acid

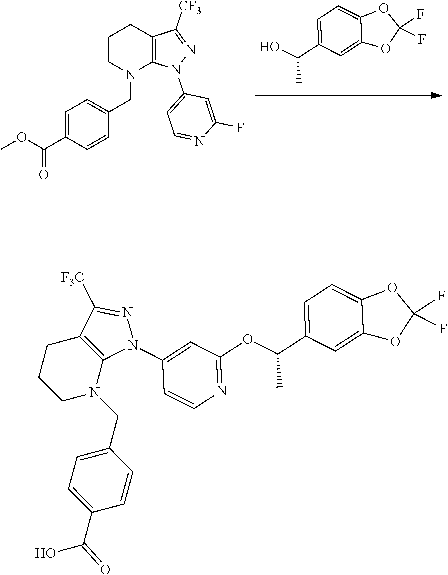 [SMILES: COC(=O)c1ccc(CN2CCCc3c(C(F)(F)F)nn(-c4ccnc(F)c4)c32)cc1 ; C[C@H](O)c1ccc2c(c1)OC(F)(F)O2 ; C[C@H](Oc1cc(-n2nc(C(F)(F)F)c3c2N(Cc2ccc(C(=O)O)cc2)CCC3)ccn1)c1ccc2c(c1)OC(F)(F)O2]

To the solution of (1S)-1-(2,2-difluoro-1,3-benzodioxol-5-yl)ethanol (Intermediate C-1S) (67 mg, 0.331 mmol) in THF (2 mL) was added NaH (14 mg, 0.55 mmol) and the solution was stirred at rt for 1 h. Then methyl 4-[[1-(2-fluoro-4-pyridyl)-3-(trifluoromethyl)-5,6-dihydro-4H-pyrazolo[3,4-b]pyridine-7-yl]methyl]benzoate (product from Step 1) (120 mg, 0.276 mmol) was added to the solution and stirred at rt for 12 h. Water (20 mL) was added and extracted with EA (2×20 mL). The organics were then separated, dried (Na₂SO₄), and then concentrated to dryness. The crude product was then purified by prep-TLC (EA/MeOH=15/1) to get the title compound (21 mg, 12% yield) as a white solid.

LC-MS (Method 1): Retention time=1.95 min. MS (ESI) m/z 603.1 [M+H]⁺.

¹H NMR (500 MHZ, CH₃OH-d₄) δ 8.07 (d, J=5.6 Hz, 1H), 8.01 (d, J=7.9 Hz, 2H), 7.42 (d, J=7.8 Hz, 2H), 7.35 (s, 1H), 7.32 (d, J=5.6 Hz, 1H), 7.08 (s, 1H), 7.06-6.99 (m, 2H), 6.07 (q, J=6.5 Hz, 1H), 4.10-3.97 (m, 2H), 3.24-3.19 (m, 2H), 2.71-2.62 (m, 2H), 1.86-1.71 (m, 2H), 1.51 (d, J=6.5 Hz, 3H).

### Example 9

### 4-[[1-[2-[(1S)-1-(2,2-difluoro-1,3-benzodioxol-5-yl)ethoxy]-4-pyridyl]-3-(trifluoromethyl)-5,6-dihydro-4H-pyrazolo[3,4-b]pyridine-7-yl]methyl]bicyclo[2.2.2]octane-1-carboxylic acid

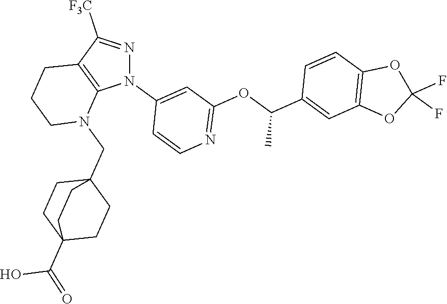 [SMILES: C[C@H](Oc1cc(-n2nc(C(F)(F)F)c3c2N(CC24CCC(C(=O)O)(CC2)CC4)CCC3)ccn1)c1ccc2c(c1)OC(F)(F)O2]

Example 9 was synthesized by using the protocol similar to Example 8

LC-MS (Method 2): Retention time=1.89 min. MS (ESI) m/z 635.3 [M+H]⁺.

¹H NMR (400 MHZ, CH₃OH-d₄) δ 8.20 (d, J=5.6 Hz, 1H), 7.32 (s, 1H), 7.25 (d, J=8.0 Hz, 1H), 7.11-7.16 (m, 2H), 7.04 (s, 1H), 6.24 (q, J=6.4 Hz, 1H), 3.30-3.33 (m 2H), 2.62 (t, J=6.4 Hz, 2H), 2.56 (d, J=4.4 Hz, 2H), 1.82-1.85 (m, 2H), 1.61-1.65 (m, 9H), 1.22-1.26 (m, 6H).

### Example 10

### 3-[[1-[3-[1-(2,2-difluoro-1,3-benzodioxol-5-yl)ethoxy]phenyl]-3-(trifluoromethyl)-5,6-dihydro-4H-pyrazolo[3.4-b]pyridine-7-yl]methyl]bicyclo[1.1.1]pentane-1-carboxylic acid

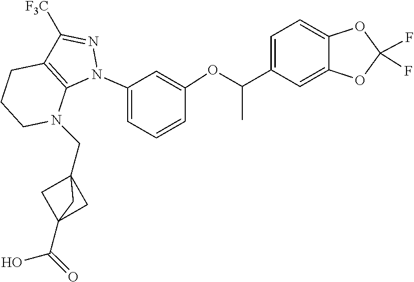 [SMILES: CC(Oc1cccc(-n2nc(C(F)(F)F)c3c2N(CC24CC(C(=O)O)(C2)C4)CCC3)c1)c1ccc2c(c1)OC(F)(F)O2]

### Step 1. Synthesis of 1-[3-[1-(2,2-difluoro-1,3-benzodioxol-5-yl)ethoxy]phenyl]-3-(trifluoromethyl)-4,5,6,7-tetrahydropyrazolo[3,4-b]pyridine

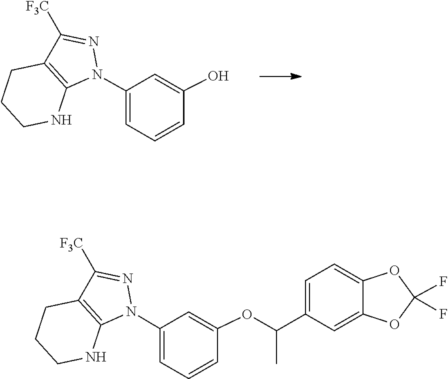 [SMILES: Oc1cccc(-n2nc(C(F)(F)F)c3c2NCCC3)c1 ; CC(Oc1cccc(-n2nc(C(F)(F)F)c3c2NCCC3)c1)c1ccc2c(c1)OC(F)(F)O2]

To a solution of 3-[3-(trifluoromethyl)-4,5,6,7-tetrahydropyrazolo[3,4-b]pyridine-1-yl]phenol (Intermediate B-1) (300 mg, 1.06 mmol), 1-(2,2-difluoro-1,3-benzodioxol-5-yl)ethanol (Intermediate C-1) (321 mg, 1.59 mmol) and triphenylphosphine (556 mg, 2.12 mmol) in THF (5 mL) was added diisopropyl azodicarboxylate (0.41 mL, 2.12 mmol) at 0° C. under Ar. The reaction mixture was stirred at 0° C. for 2 h. Then water (30 mL) was added. The solution was extracted with EA (2×30 mL), washed with brine (30 mL), dried over Na₂SO₄, and concentrated in vacuum. The residue was purified by Combi-flash (A: water (10 mmol/L NH₄HCO₃) B: ACN) to yield the title compound (330 mg, 65% yield) as a yellow solid.

LC-MS (Method 1): Retention time=2.39 min. MS (ESI) m/z 468.0 [M+H]⁺.

### Step 2. Synthesis of methyl 3-[[1-[3-[1-(2,2-difluoro-1,3-benzodioxol-5-yl)ethoxy]phenyl]-3-(trifluoromethyl)-5,6-dihydro-4H-pyrazolo[3,4-b]pyridine-7-yl]methyl]bicyclo[1.1.1]pentane-1-carboxylate

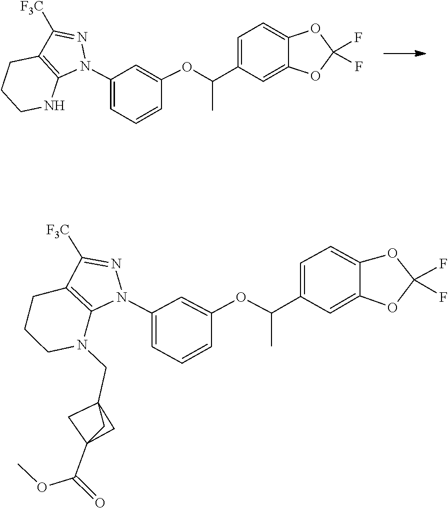 [SMILES: CC(Oc1cccc(-n2nc(C(F)(F)F)c3c2NCCC3)c1)c1ccc2c(c1)OC(F)(F)O2 ; COC(=O)C12CC(CN3CCCc4c(C(F)(F)F)nn(-c5cccc(OC(C)c6ccc7c(c6)OC(F)(F)O7)c5)c43)(C1)C2]

To a solution of methyl 3-[[1-(3-hydroxyphenyl)-3-(trifluoromethyl)-5,6-dihydro-4H-pyrazolo[3,4-b]pyridine-7-yl]methyl]bicyclo[1.1.1]pentane-1-carboxylate (110 mg, 0.261 mmol) and methyl 3-(bromomethyl)bicyclo[1.1.1]pentane-1-carboxylate (158 mg, 0.720 mmol) in THF (10 mL) was added potassium tert-butoxide (1.8 mL, 1.80 mmol) at 0° C. The reaction was stirred at rt for 2 h. Then water (30 mL) was added. The solution was extracted with EA (2×30 mL), washed with brine (30 mL), dried over Na₂SO₄, and concentrated in vacuum. The residue was purified by Combi-flash (A: water (10 mmol/L NH₄HCO₃); B: ACN) to yield the title compound (60 mg, 36% yield) as a yellow solid.

LC-MS (Method 1): Retention time=2.54 min. MS (ESI) m/z 606.0 [M+H]⁺.

### Step 3. Synthesis of 3-[[1-[3-[1-(2,2-difluoro-1,3-benzodioxol-5-yl)ethoxy]phenyl]-3-(trifluoromethyl)-5,6-dihydro-4H-pyrazolo[3,4-b]pyridine-7-yl]methyl]bicyclo[1.1.1]pentane-1-carboxylic acid

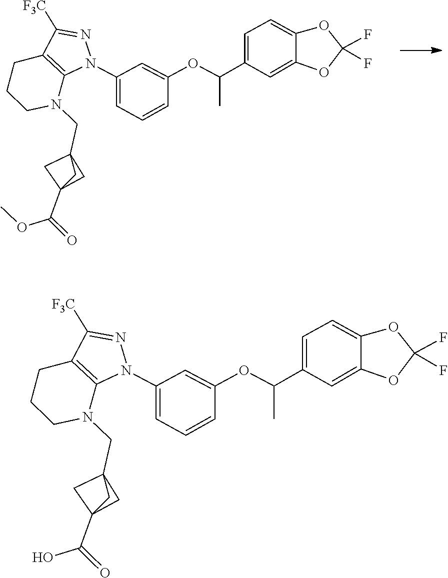 [SMILES: COC(=O)C12CC(CN3CCCc4c(C(F)(F)F)nn(-c5cccc(OC(C)c6ccc7c(c6)OC(F)(F)O7)c5)c43)(C1)C2 ; CC(Oc1cccc(-n2nc(C(F)(F)F)c3c2N(CC24CC(C(=O)O)(C2)C4)CCC3)c1)c1ccc2c(c1)OC(F)(F)O2]

To a solution of methyl 3-[[1-[3-[1-(2,2-difluoro-1,3-benzodioxol-5-yl)ethoxy]phenyl]-3-(trifluoromethyl)-5,6-dihydro-4H-pyrazolo[3,4-b]pyridine-7-yl]methyl]bicyclo[1.1.1]pentane-1-carboxylate (11 mg, 0.0186 mmol) in methanol (1 mL), THF (1 mL) and water (0.5 mL) was added lithium hydroxide monohydrate (2.3 mg, 0.0559 mmol), and the resulting mixture was stirred at rt for 4 h. The reaction mixture was then concentrated in vacuum. The residue was purified by Prep-HPLC (A: water (10 mmol/L NH₄HCO₃); B: ACN) to yield the title compound (6.0 mg, 52% yield) as a white solid.

LC-MS (Method 1): Retention time=1.87 min. MS (ESI) m/z 592.2 [M+H]⁺.

### Example 11

### 3-[[1-[3-[(1S)-1-(2,2-difluoro-1,3-benzodioxol-5-yl)ethoxy]phenyl]-3-(trifluoromethyl)-5,6-dihydro-4H-pyrazolo[3,4-b]pyridine-7-yl]methyl]bicyclo[1.1.1]pentane-1-carboxylic acid

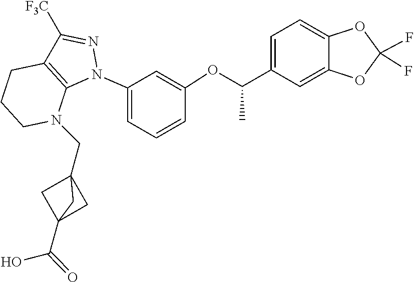 [SMILES: C[C@H](Oc1cccc(-n2nc(C(F)(F)F)c3c2N(CC24CC(C(=O)O)(C2)C4)CCC3)c1)c1ccc2c(c1)OC(F)(F)O2]

### Step 1. Synthesis of 1-[3-[(1S)-1-(2,2-difluoro-1,3-benzodioxol-5-yl)ethoxy]phenyl]-3-(trifluoromethyl)-4,5,6,7-tetrahydropyrazolo[3,4-b]pyridine

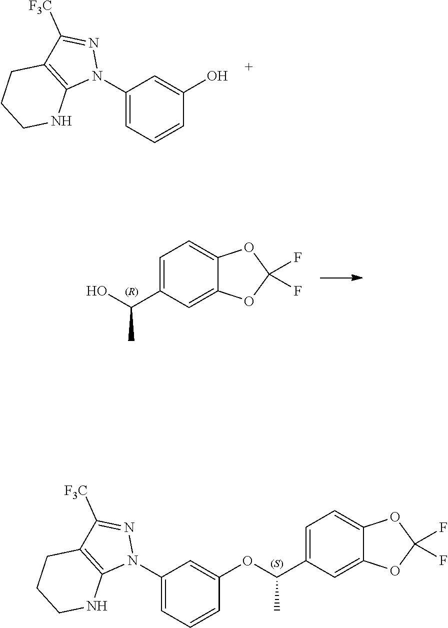

To a solution of 3-[3-(trifluoromethyl)-4,5,6,7-tetrahydropyrazolo[3,4-b]pyridine-1-yl]phenol (Intermediate B-1) (100 mg, 0.353 mmol), (1R)-1-(2,2-difluoro-1,3-benzodioxol-5-yl)ethanol (Intermediate C-1R) (86 mg, 0.424 mmol) and triphenylphosphine (139 mg, 0.530 mmol) in THF (5 mL), was added diisopropyl azodicarboxylate (0.10 mL, 0.530 mmol) at 0° C. under N₂. The reaction was stirred at rt for 4 h. Then water (20 mL) was added. The resulting mixture was extracted with EA (3×20 mL). The combined organic extracts were combined and washed with brine (20 mL), dried over Na₂SO₄, filtered, and concentrated in vacuum. The residue was purified by silica gel column chromatography (PE:EA=2:1) to yield the title compound (100 mg, 61% yield) as a yellow solid.

LC-MS (Method 1): Retention time=1.94 min. MS (ESI) m/z 468.0 [M+H]⁺.

### Step 2. Synthesis of 3-[[1-[3-[(1S)-1-(2,2-difluoro-1,3-benzodioxol-5-yl)ethoxy]phenyl]-3-(trifluoromethyl)-5,6-dihydro-4H-pyrazolo[3,4-b]pyridine-7-yl]methyl]bicyclo[1.1.1]pentane-1-carboxylic acid

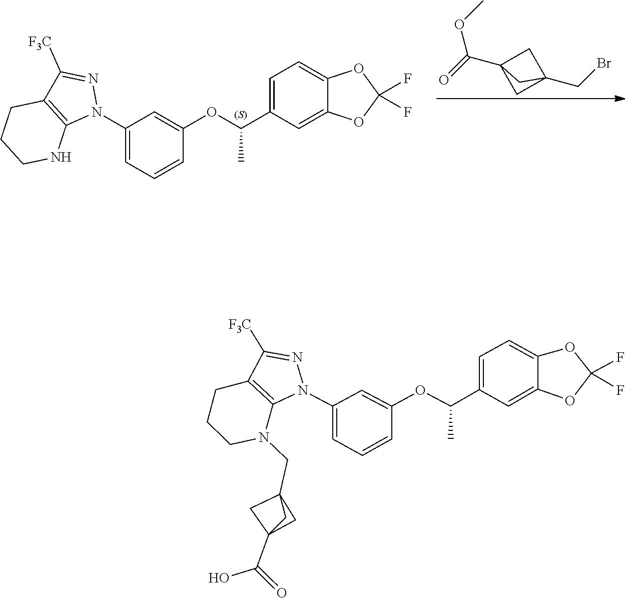

To a solution of 1-[3-[(1S)-1-(2,2-difluoro-1,3-benzodioxol-5-yl)ethoxy]phenyl]-3-(trifluoromethyl)-4,5,6,7-tetrahydropyrazolo[3,4-b]pyridine (100 mg, 0.214 mmol) and methyl 3-(bromomethyl)bicyclo[1.1.1]pentane-1-carboxylate (52 mg, 0.235 mmol) in THF (2 mL) was added potassium tert-butoxide (0.53 mL, 0.535 mmol), and the resulting mixture stirred at rt for 14 h. Then water (20 mL) was added and extracted with EA (3×20 mL). The EA solution was washed with brine (20 mL), dried over Na₂SO₄, filtered, and concentrated in vacuum. The residue was purified by prep-HPLC (A: water (10 mmol/L NH₄HCO₃); B: ACN) to yield the title compound (30 mg, 24% yield) as a white solid.

LC-MS (Method 2): Retention time=1.865 min. MS (ESI) m/z 591.8 [M+H]⁺.

¹H NMR (400 MHZ, CH₃OH-d₄) 8:7.36 (t, J=8.1 Hz, 1H), 7.29 (s, 1H), 7.27-6.90 (m, 5H), 5.50 (q, J=6.3 Hz, 1H), 3.23-3.15 (m, 2H), 2.62 (dd, J=13.9, 6.6 Hz, 4H), 1.88-1.74 (m, 2H), 1.65 (s, 6H), 1.62 (d, J=6.3 Hz, 3H).

### Example 12

### (S)-4-((1-(5-(1-(2,2-difluorobenzo[d][1,3]dioxol-5-yl)ethoxy)pyridin-3-yl)-3-(trifluoromethyl)-1,4,5,6-tetrahydro-7H-pyrazolo[3,4-b]pyridin-7-yl)methyl)benzoic acid

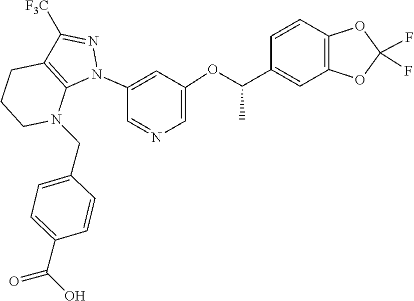 [SMILES: C[C@H](Oc1cncc(-n2nc(C(F)(F)F)c3c2N(Cc2ccc(C(=O)O)cc2)CCC3)c1)c1ccc2c(c1)OC(F)(F)O2]

The title compound was synthesized essentially in the same manner as Example 11.

Chiral Method: Column Name: (R, R) WHELK-O1 4.6×100 mm 3.5 μm Solvent: MeOH [0.2% NH₃ (7M in MeOH)]; Retention time=2.963 min; ee=100%.

LC-MS (Method 3): Retention time=1.86 min. MS (ESI) m/z 603.1 [M+H]⁺.

¹H NMR (400 MHZ, CH₃OH-d₄) δ:8.44 (S, 1H), 8.17 (S, 1H), 7.89 (d, J=8 Hz, 2H), 7.67 (s, 1H), 7.24 (s, 1H), 7.13-7.08 (m, 4H), 5.43 (dd, J=12.4 Hz, 6 Hz, 1H), 3.81 (dd, J=23.2 Hz, 7.6 Hz, 2H), 3.18 (t, J=6 Hz, 2H), 2.62 (t, J=Hz, 2H), 1.72-1.68 (m, 2H), 1.59 (d, J=6.4 Hz, 3H).

### Example 13

### Trans-4-[[1-[5-[(1S)-1-(2,2-difluoro-1,3-benzodioxol-5-yl)ethoxy]-3-pyridyl]-3-(trifluoromethyl)-5,6-dihydro-4H-pyrazolo[3,4-b]pyridine-7-yl]methyl]cyclohexanecarboxylic acid

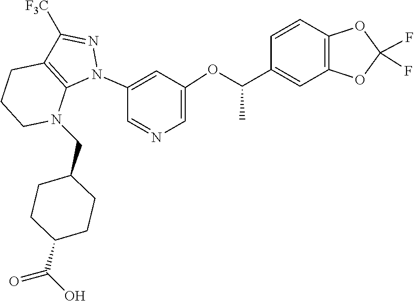 [SMILES: C[C@H](Oc1cncc(-n2nc(C(F)(F)F)c3c2N(C[C@H]2CC[C@H](C(=O)O)CC2)CCC3)c1)c1ccc2c(c1)OC(F)(F)O2]

The title compound was synthesized essentially in the same manner as Example 11.

Chiral Method: Column Name: AS-3 4.6×100 mm 3 μm. Solvent: MeOH [0.2% NH₃ (7M in MeOH)]; Retention time=1.467 min; ee=100%.

LC-MS (Method 2): Retention time=1.67 min. MS (ESI) m/z 608.8 [M+H]⁺.

¹H NMR (400 MHZ, CH₃OH-d₄) δ:8.32 (dd, J=6.8 Hz, 4.4 Hz, 2H), 7.58-7.67 (m, 1H), 7.35 (s, 1H), 7.29-7.27 (m, 1H), 7.22-7.20 (m, 1H), 5.62 (dd, J=6.8 Hz, 1H), 3.19-3.17 (m, 2H), 2.63 (t, J=6.0 Hz, 1H), 2.33 (d, J=7.2 Hz, 2H), 1.93-1.79 (m, 5H), 1.67 (d, J=6.0 Hz, 3H), 1.49-1.46 (m, 2H), 1.33-1.24 (m, 3H), 0.41-0.38 (m, 2H).

### Example 14

### (S)-4-((1-(5-(1-(2,2-difluorobenzo[d][1,3]dioxol-5-yl)ethoxy)pyridin-3-yl)-3-(trifluoromethyl)-1,4,5,6-tetrahydro-7H-pyrazolo[3,4-b]pyridin-7-yl)methyl)bicyclo[2.2.2]octane-1-carboxylic acid

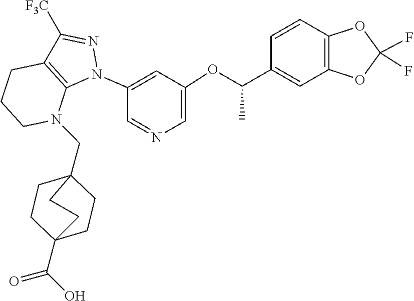 [SMILES: C[C@H](Oc1cncc(-n2nc(C(F)(F)F)c3c2N(CC24CCC(C(=O)O)(CC2)CC4)CCC3)c1)c1ccc2c(c1)OC(F)(F)O2]

The title compound was synthesized essentially in the same manner as Example 11.

LC-MS (Method 4): Retention time=1.83 min. MS (ESI) m/z 635.2 [M+H]⁺.

¹H NMR (400 MHZ, CH₃OH-d₄) δ:8.38 (d, J=2.4 Hz, 1H), 8.22 (d, J=2.0 Hz, 1H), 7.46 (t, J=2.4 Hz, 1H), 7.37 (d, J=2.4 Hz, 1H), 7.31-7.28 (m, 1H), 7.22-7.20 (m, 1H), 5.62 (dd, J=12.8 Hz, 6.4 Hz, 4H), 3.20-3.17 (m, 2H), 2.62 (t, J=6.4 Hz, 2H), 2.32 (dd, J=28.4 Hz, 13.6 Hz, 2H), 1.82-1.80 (m, 2H), 1.67 (d, J=6.0 Hz, 3H), 1.56 (t, J=8.0 Hz, 6H), 1.00-0.96 (m, 6H).

### Example 15

### (S)-4-(1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine-7-carbonyl)benzoic acid

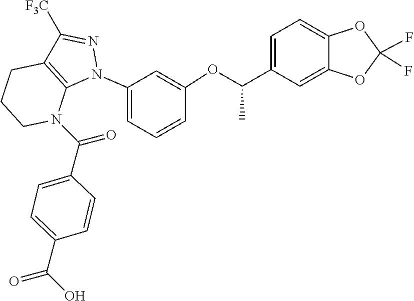 [SMILES: C[C@H](Oc1cccc(-n2nc(C(F)(F)F)c3c2N(C(=O)c2ccc(C(=O)O)cc2)CCC3)c1)c1ccc2c(c1)OC(F)(F)O2]

### Step 1. Synthesis of methyl(S)-4-(1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine-7-carbonyl)benzoate

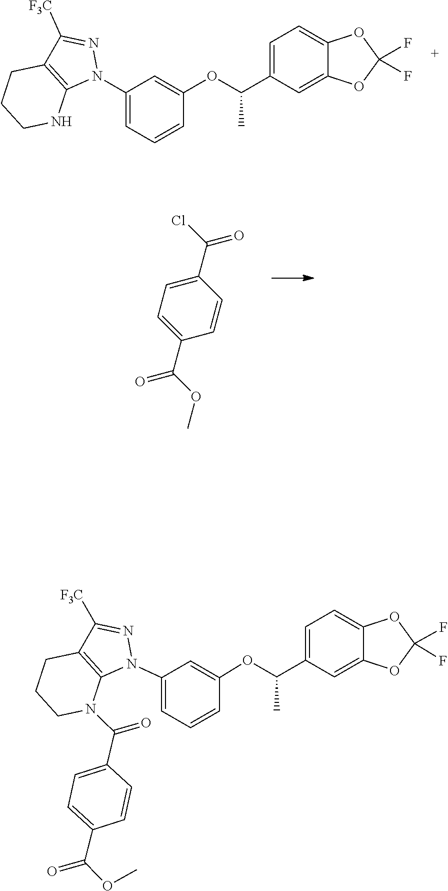 [SMILES: C[C@H](Oc1cccc(-n2nc(C(F)(F)F)c3c2NCCC3)c1)c1ccc2c(c1)OC(F)(F)O2 ; COC(=O)c1ccc(C(=O)Cl)cc1 ; COC(=O)c1ccc(C(=O)N2CCCc3c(C(F)(F)F)nn(-c4cccc(O[C@@H](C)c5ccc6c(c5)OC(F)(F)O6)c4)c32)cc1]

To a solution of(S)-1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine (product of Step 1, Example 11) (250 mg, 0.40 mmol) in pyridine (2 mL) was added methyl 4-(chlorocarbonyl)benzoate (118 mg, 0.60 mmol) and DMAP (49 mg, 0.40 mmol). The solution was stirred at 100° C. for 48 h. Then water (20 mL) was added and extracted with EA (2×20 mL). The organic phase was washed with brine and dried over Na₂SO₄. The organics were concentrated under vacuum. The residue was purified by flash column chromatography (PE:EA=2:1) to give the title compound (50 mg, 15% yield) as a white solid.

LC-MS (Method 4): Retention time=2.37 min. MS (ESI) m/z 630.1 [M+H]⁺.

### Step 2. Synthesis of(S)-4-(1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine-7-carbonyl)benzoic acid

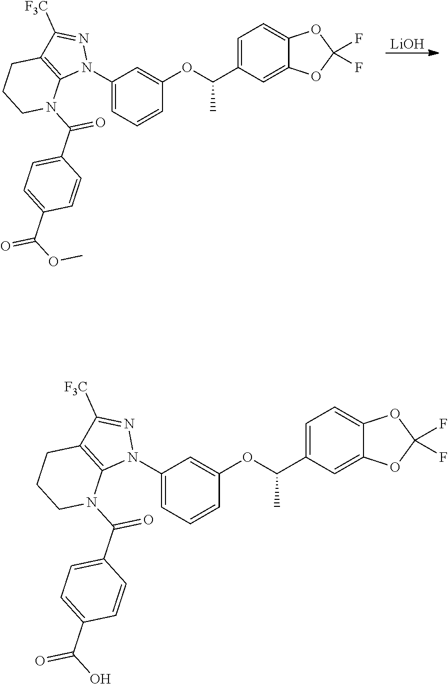

To a solution of methyl(S)-4-(1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine-7-carbonyl)benzoate (50 mg, 0.08 mmol) in MeOH (2 mL), THF (2 mL) and H₂O (1 mL) was added LiOH·H₂O (6 mg, 0.16 mmol). The solution was stirred at rt for 1 h. Then the solution was quenched with 1N aqueous HCl to adjust the pH to 3 and extracted with EA (3×30 mL). The organic phase was washed with brine, dried over Na₂SO₄, and concentrated under vacuum. The residue was purified by prep-HPLC to the title compound (18.5 mg, 39% yield) as a white solid.

LC-MS (Method 2): Retention time=1.62 min. MS (ESI) m/z 615.7 [M+H]⁺.

¹H NMR (500 MHZ, DMSO-d₆, T60) δ:7.85 (d, J=7.6 Hz, 2H), 7.35-7.27 (m, 4H), 7.20-7.13 (m, 2H), 6.84-6.82 (m, 3H), 5.44 (dd, J=12.4, 6.0 Hz, 1H), 3.91 (br, 2H), 2.77 (t, J=6.4 Hz, 2H), 1.95-1.93 (m, 2H), 1.49 (d, J=6.4 Hz, 3H).

### Example 16

### (S)-4-(1-(5-(1-(2,2-difluorobenzo[d][1,3]dioxol-5-yl)ethoxy)pyridin-3-yl)-3-(trifluoromethyl)-4,5,6,7-tetrahydro-1H-pyrazolo[3,4-b]pyridine-7-carbonyl)benzoic acid

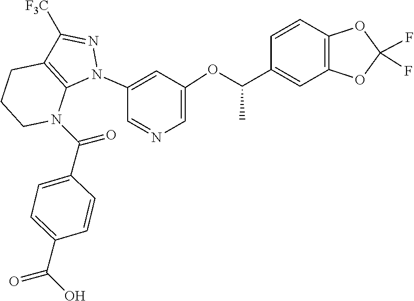 [SMILES: C[C@H](Oc1cncc(-n2nc(C(F)(F)F)c3c2N(C(=O)c2ccc(C(=O)O)cc2)CCC3)c1)c1ccc2c(c1)OC(F)(F)O2]

The title compound was synthesized essentially in the same manner as Example 15.

LC-MS (Method 4): Retention time=1.60 min. MS (ESI) m/z 617.2 [M+H]⁺.

¹H NMR (400 MHZ, DMSO-d₆, T60) δ:8.23-8.19 (m, 2H), 7.92 (d, J=8.0 Hz, 2H), 7.44-7.32 (m, 5H), 7.25 (d, J=6.8 Hz, 1H), 5.61 (dd, J=12.4 Hz, 6.4 Hz, 1H), 4.00-3.89 (m, 2H), 2.80 (t, J=6.4 Hz, 2H), 1.96 (t, J=5.6 Hz, 2H), 1.55 (d, J=6.4 Hz, 3H).

### Example 17

Trans-4-[1-[5-[(1S)-1-(2,2-difluoro-1,3-benzodioxol-5-yl)ethoxy]-3-pyridyl]-3-(trifluoromethyl)-5,6-dihydro-4H-pyrazolo[3,4-b]pyridine-7-carbonyl]cyclohexanecarboxylic acid

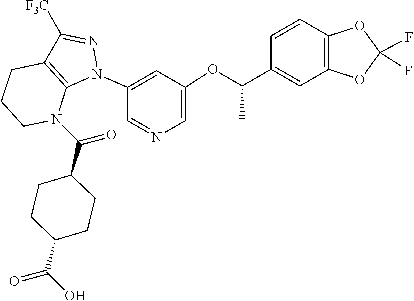 [SMILES: C[C@H](Oc1cncc(-n2nc(C(F)(F)F)c3c2N(C(=O)[C@H]2CC[C@H](C(=O)O)CC2)CCC3)c1)c1ccc2c(c1)OC(F)(F)O2]

The title compound was synthesized essentially in the same manner as Example 15.

LC-MS (Method 4): Retention time=1.60 min. MS (ESI) m/z 623.2 [M+H]⁺.

¹H NMR (400 MHZ, DMSO-d₆) δ:8.30 (dd, J=13.6 Hz, 10.8 Hz, 2H), 7.52-7.50 (m, 2H), 7.40-7.32 (m, 2H), 5.71 (dd, J=12.4 Hz, 6.0 Hz, 1H), 3.96 (br, 2H), 2.75 (t, J=6.4 Hz, 1H), 2.54 (br, 1H), 2.10-2.05 (m, 1H), 1.94-1.83 (m, 4H), 1.63-1.13 (m, 7H), 1.15-1.09 (m, 2H).

### Example 18

### (S)-1-(1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine-7-carbonyl) piperidine-4-carboxylic acid

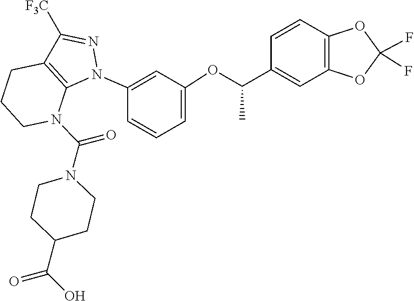 [SMILES: C[C@H](Oc1cccc(-n2nc(C(F)(F)F)c3c2N(C(=O)N2CCC(C(=O)O)CC2)CCC3)c1)c1ccc2c(c1)OC(F)(F)O2]

### Step 1. Synthesis of trichloromethyl(S)-1-(3-(1-(2,2-difluorobenzo[d][1,3]dioxol-5-yl)ethoxy)phenyl)-3-(trifluoromethyl)-1,4,5,6-tetrahydro-7H-pyrazolo[3,4-b]pyridine-7-carboxylate

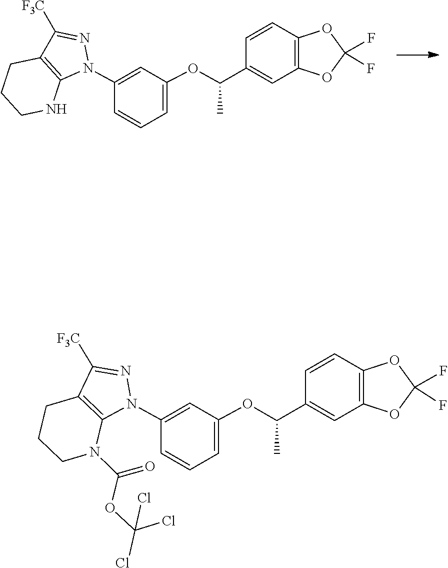 [SMILES: C[C@H](Oc1cccc(-n2nc(C(F)(F)F)c3c2NCCC3)c1)c1ccc2c(c1)OC(F)(F)O2 ; C[C@H](Oc1cccc(-n2nc(C(F)(F)F)c3c2N(C(=O)OC(Cl)(Cl)Cl)CCC3)c1)c1ccc2c(c1)OC(F)(F)O2]

To a solution of(S)-1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine (product of step 1, Example 11) (100 mg, 0.21 mmol) in anhydrous THF (5 mL) was added triphosgene (424 mg, 1.07 mmol) and TEA (108 mg, 1.07 mmol). The solution was stirred at rt for 18 h. Then the solution was concentrated under vacuum to give crude trichloromethyl(S)-1-(3-(1-(2,2-difluorobenzo[d][1,3]dioxol-5-yl)ethoxy)phenyl)-3-(trifluoromethyl)-1,4,5,6-tetrahydro-7H-pyrazolo[3,4-b]pyridine-7-carboxylate as a yellow oil.

### Step 2. Synthesis of methyl(S)-1-(1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine-7-carbonyl) piperidine-4-carboxylate

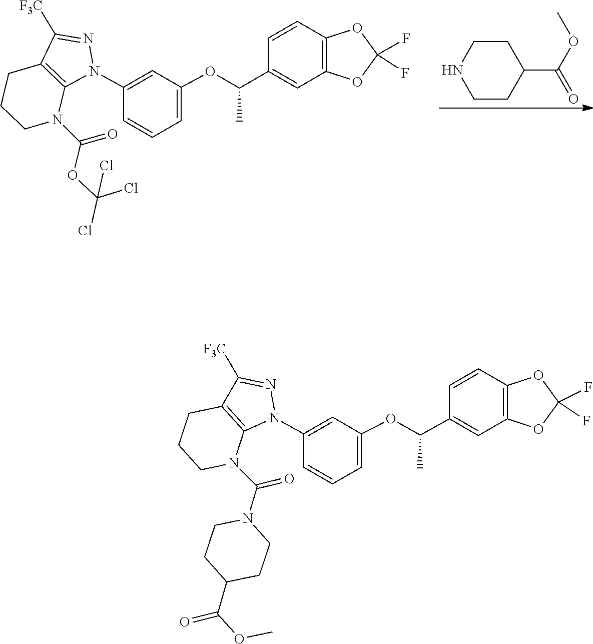

To a solution of trichloromethyl(S)-1-(3-(1-(2,2-difluorobenzo[d][1,3]dioxol-5-yl)ethoxy)phenyl)-3-(trifluoromethyl)-1,4,5,6-tetrahydro-7H-pyrazolo[3,4-b]pyridine-7-carboxylate (crude obtained in Step 1) in DCM (5 mL) was added methyl piperidine-4-carboxylate (153 mg, 1.07 mmol) and TEA (108 mg, 1.07 mmol). The solution was stirred at rt for 1 h. Water (20 mL) was then added, and the mixture extracted with EA (2×20 mL). The organic phase was washed with brine and dried over Na₂SO₄. The organics were concentrated under vacuum. The residue was purified by flash column chromatography (PE:EA=1:1) to give the title compound (70 mg, 52% yield) as a white solid.

LC-MS (Method 2): Retention time=2.30 min. MS (ESI) m/z 637.2 [M+H]⁺.

### Step 3. Synthesis of(S)-1-(1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine-7-carbonyl) piperidine-4-carboxylic acid

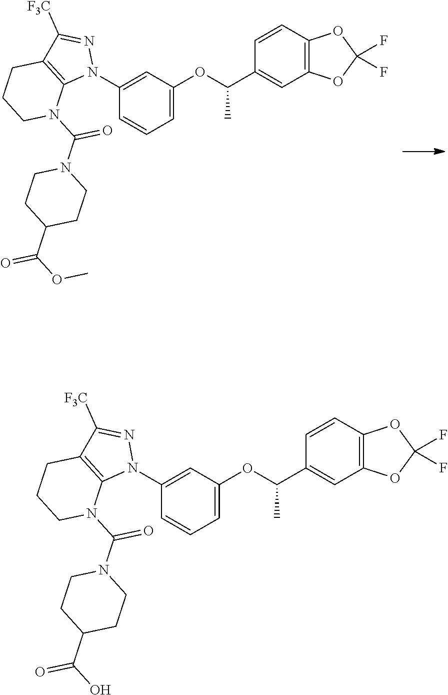 [SMILES: COC(=O)C1CCN(C(=O)N2CCCc3c(C(F)(F)F)nn(-c4cccc(O[C@@H](C)c5ccc6c(c5)OC(F)(F)O6)c4)c32)CC1 ; C[C@H](Oc1cccc(-n2nc(C(F)(F)F)c3c2N(C(=O)N2CCC(C(=O)O)CC2)CCC3)c1)c1ccc2c(c1)OC(F)(F)O2]

To a solution of methyl(S)-1-(1-(3-(1-(2,2-difluorobenzo[d][1,3]dioxol-5-yl)ethoxy)phenyl)-3-(trifluoromethyl)-4,5,6,7-tetrahydro-1H-pyrazolo[3,4-b]pyridine-7-carbonyl) piperidine-4-carboxylate (70 mg, 0.11 mmol) in MeOH (2 mL), THF (2 mL) and H₂O (1 mL) was added LiOH H₂O (9 mg, 0.22 mmol). The solution was stirred at rt for 1 h. Then the solution was quenched with 1N aqueous HCl to adjust the pH to 3 and extracted with EA (3×30 mL). The organic phase was washed with brine and dried over Na₂SO₄.

The organics were concentrated under vacuum. The residue was purified by prep-HPLC to give the title compound (33.4 mg, 49% yield) as a white solid.

LC-MS (Method 2): Retention time=1.72 min. MS (ESI) m/z 623.1 [M+H]⁺.

¹H NMR (400 MHZ, CH₃OH-d₄) δ:7.31-7.27 (m, 2H), 7.23-7.21 (m, 1H), 7.15-7.13 (m, 1H), 7.04-6.98 (m, 2H), 6.91-6.88 (m, 1H), 5.49 (dd, J=12.8 Hz, 6.4 Hz, 1H), 3.80 (br, 2H), 3.61 (br, 2H), 2.89 (br, 2H), 2.72 (t, J=6.4 Hz, 2H), 2.45-2.39 (m, 1H), 1.87-1.76 (m, 4H), 1.60 (d, J=6.8 Hz, 3H), 1.46 (br, 2H).

### Example 19

### 4-((1-(3-(1-(2,2-difluorobenzo[d][1,3]dioxol-5-yl)ethoxy)phenyl)-3-(trifluoromethyl)-4,5,6,7-tetrahydro-1H-indazol-7-yl)oxy)benzoic acid

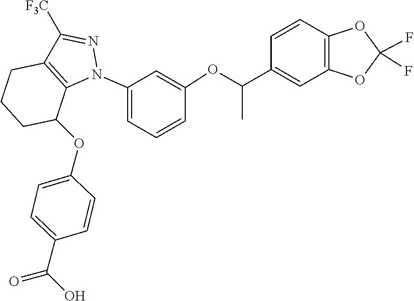 [SMILES: CC(Oc1cccc(-n2nc(C(F)(F)F)c3c2C(Oc2ccc(C(=O)O)cc2)CCC3)c1)c1ccc2c(c1)OC(F)(F)O2]

### Step 1. Synthesis of tert-butyl 4-((1-(3-bromophenyl)-3-(trifluoromethyl)-4,5,6,7-tetrahydro-1H-indazol-7-yl)oxy)benzoate

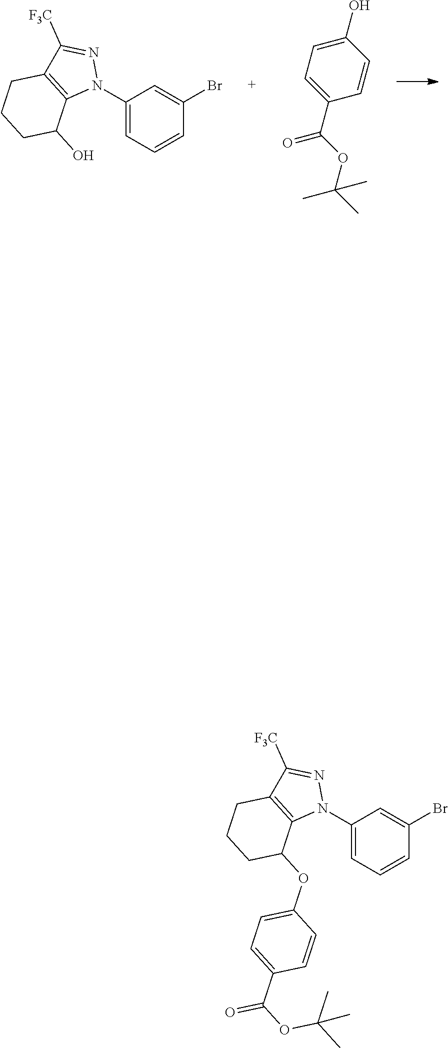 [SMILES: OC1CCCc2c(C(F)(F)F)nn(-c3cccc(Br)c3)c21 ; CC(C)(C)OC(=O)c1ccc(O)cc1 ; CC(C)(C)OC(=O)c1ccc(OC2CCCc3c(C(F)(F)F)nn(-c4cccc(Br)c4)c32)cc1]

To the solution of 1-(3-bromophenyl)-3-(trifluoromethyl)-4,5,6,7-tetrahydroindazol-7-ol (Intermediate D-2b) (720 mg, 1.99 mmol), tert-butyl 4-hydroxybenzoate (581 mg, 2.99 mmol) and triphenylphosphine (1046 mg, 3.99 mmol) in THF (20 mL) was added DIAD (0.78 mL, 3.99 mmol) dropwise at 0° C. The solution was then stirred at rt overnight. The solution was poured into water (50 mL) and extracted with EA (3×30 mL). The organics were washed with 30 mL of brine. The organics were combined and dried (Na₂SO₄) before concentrating to dryness. The crude product was then purified by flash column chromatography eluting with 10% ethyl acetate in isohexane to get the title compound (490 mg, 46% yield) as a yellow solid.

LC-MS (Method 1): Retention time=2.63 min. MS (ESI) m/z 559/561 [M+Na]⁺.

### Step 2. Synthesis of tert-butyl 4-((1-(3-hydroxyphenyl)-3-(trifluoromethyl)-4,5,6,7-tetrahydro-1H-indazol-7-yl)oxy)benzoate

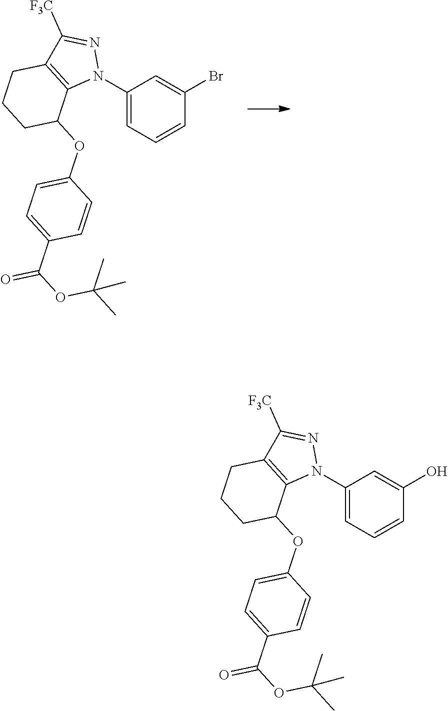 [SMILES: CC(C)(C)OC(=O)c1ccc(OC2CCCc3c(C(F)(F)F)nn(-c4cccc(Br)c4)c32)cc1 ; CC(C)(C)OC(=O)c1ccc(OC2CCCc3c(C(F)(F)F)nn(-c4cccc(O)c4)c32)cc1]

To the solution of tert-butyl 4-[[1-(3-bromophenyl)-3-(trifluoromethyl)-4,5,6,7-tetrahydroindazol-7-yl]oxy]benzoate (440 mg, 0.819 mmol) in 1,4-dioxane (3.5 mL) and water (1.5 mL) were added KOH (92 mg, 1.64 mmol), tris(dibenzylideneacetone) dipalladium (75 mg, 0.0819 mmol), and 2-di-tert-butylphosphino-2′,4′,6′-triisopropylbiphenyl (35 mg, 0.0819 mmol). The solution was stirred at 100° C. under N₂ for 1 h. The solution was poured into water (30 mL) and extracted with EA (3×20 mL). The organic layers were combined, washed with brine (30 mL), dried over Na₂SO₄, filtered and concentrated. The crude product was purified by flash column chromatography eluted with 10% ethyl acetate in isohexane to get the title compound (270 mg, 69% yield) as a white solid.

LC-MS (Method 2): Retention time=2.06 min. MS (ESI) m/z 419.1 (M-tert-butyl)⁺.

### Step 3. Synthesis of tert-butyl 4-((1-(3-(1-(2,2-difluorobenzo[d][1,3]dioxol-5-yl)ethoxy)phenyl)-3-(trifluoromethyl)-4,5,6,7-tetrahydro-1H-indazol-7-yl)oxy)benzoate

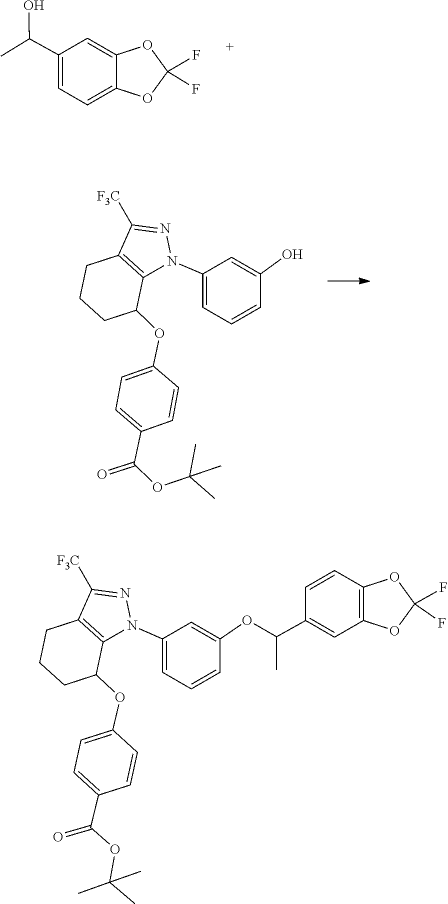 [SMILES: CC(O)c1ccc2c(c1)OC(F)(F)O2 ; CC(C)(C)OC(=O)c1ccc(OC2CCCc3c(C(F)(F)F)nn(-c4cccc(O)c4)c32)cc1 ; CC(Oc1cccc(-n2nc(C(F)(F)F)c3c2C(Oc2ccc(C(=O)OC(C)(C)C)cc2)CCC3)c1)c1ccc2c(c1)OC(F)(F)O2]

To the solution of 1-(2,2-difluoro-1,3-benzodioxol-5-yl)ethanol (Intermediate C-1) (202 mg, 0.999 mmol), tert-butyl 4-[[1-(3-hydroxyphenyl)-3-(trifluoromethyl)-4,5,6,7-tetrahydroindazol-7-yl]oxy]benzoate (237 mg, 0.500 mmol) and triphenylphosphine (524 mg, 2.00 mmol) in THF (10 mL) was added DIAD (0.39 mL, 2.00 mmol) dropwise at 0° C. The solution was then stirred at rt overnight. The solution was poured into water (50 mL) and extracted with EA (3×30 mL). The organics were combined, washed with 30 mL of brine, dried (Na₂SO₄), and concentrated to dryness. The crude product was then purified by flash column chromatography eluted with 10% ethyl acetate in isohexane to get the title compound (250 mg, 41% yield) as a white solid.

LC-MS (Method 1): Retention time=2.73 min. MS (ESI) m/z 681.2 (M+Na)⁺.

¹H NMR (500 MHZ, DMSO-d₆) δ 7.81 (dd, J=13.8, 8.8 Hz, 2H), 7.33-7.19 (m, 3H), 7.15-7.02 (m, 3H), 6.98 (d, J=7.6 Hz, 2H), 6.89 (d, J=8.3 Hz, 1H), 5.83 (s, 0.5H), 5.65 (s, 0.5H), 5.40-5.30 (m, 1H), 2.79 (d, J=15.8 Hz, 1H), 2.68-2.55 (m, 1H), 2.18-2.04 (m, 1H), 1.96-1.77 (m, 3H), 1.50 (d, J=6.9 Hz, 9H), 1.37 (dd, J=11.9, 6.3 Hz, 3H). (mixture of four diastereomers).

### Step 4. Synthesis of 4-((1-(3-(1-(2,2-difluorobenzo[d][1,3]dioxol-5-yl)ethoxy)phenyl)-3-(trifluoromethyl)-4,5,6,7-tetrahydro-1H-indazol-7-yl)oxy)benzoic acid

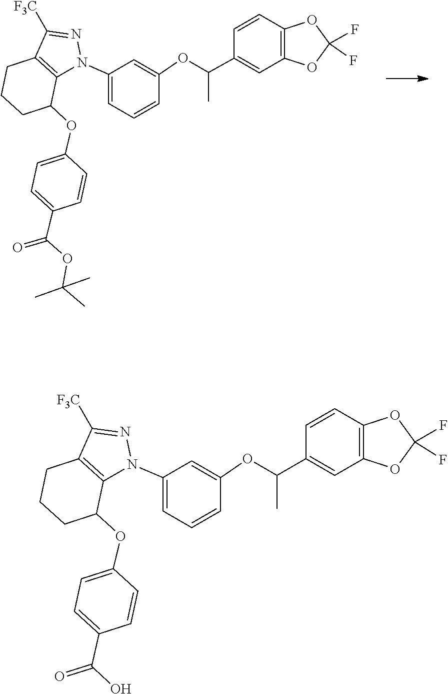 [SMILES: CC(Oc1cccc(-n2nc(C(F)(F)F)c3c2C(Oc2ccc(C(=O)OC(C)(C)C)cc2)CCC3)c1)c1ccc2c(c1)OC(F)(F)O2 ; CC(Oc1cccc(-n2nc(C(F)(F)F)c3c2C(Oc2ccc(C(=O)O)cc2)CCC3)c1)c1ccc2c(c1)OC(F)(F)O2]

To the solution of tert-butyl 4-[[1-[3-[1-(2,2-difluoro-1,3-benzodioxol-5-yl)ethoxy]phenyl]-3-(trifluoromethyl)-4,5,6,7-tetrahydroindazol-7-yl]oxy]benzoate (200 mg, 0.304 mmol) in 1,4-dioxane (10 mL) and water (3 mL) was added KOH (256 mg, 4.56 mmol) and the solution was stirred at 100° C. for 19 h. The solution was purified by prep-HPLC to get the title compound (60 mg, 33% yield) as a white solid.

LC-MS (Method 2): Retention time=1.66 min. MS (ESI) m/z 603.2 (M+H)⁺.

Δ 7.97-7/88 (m, 2H), 7.30-7.08 (m, 5H), 6.99-6.81 (m, 4H), 5.58 (s, 0.5H), 5.45 (s, 0.5H), 5.20-5.08 (m, 1H), 2.88-2.84 (m, 1H), 2.69-2.53 (m, 1H), 2.30-2.25 (m, 1H), 2.09-1.70 (m, 3H), 1.44 (d, J=6.4 Hz, 1.5H), 1.39 (d, J=6.4 Hz, 1.5H) (mixture of four diastereomers)

### Example 20

### 4-((1-(3-((2,2-difluorobenzo[d][1,3]dioxol-5-yl) methoxy)phenyl)-3-(trifluoromethyl)-4,5,6,7-tetrahydro-1H-indazol-7-yl)oxy)benzoic acid

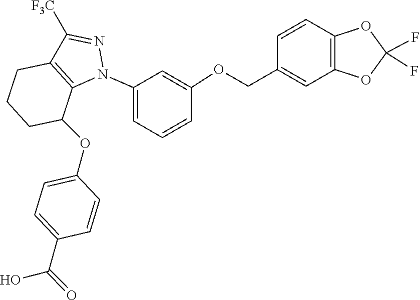 [SMILES: O=C(O)c1ccc(OC2CCCc3c(C(F)(F)F)nn(-c4cccc(OCc5ccc6c(c5)OC(F)(F)O6)c4)c32)cc1]

The title compound was synthesized essentially in the same manner as Example 19

LC-MS (Method 2): Retention time=1.61 min. MS (ESI) m/z 589.1 [M+H]⁺.

¹H NMR (400 MHZ, CH₃OH-d₄ δ 7.92 (d, J=8.8 Hz, 2H), 7.32 (t, J=8.1 Hz, 1H), 7.23-7.07 (m, 4H), 7.03-6.90 (m, 4H), 5.65 (d, J=3.2 Hz, 1H), 4.77 (d, J=11.6 Hz, 1H), 4.60 (d, J=11.6 Hz, 1H), 2.90-2.86 (m, 1H), 2.67-2.59 (m, 1H), 2.31-2.26 (m, 1H), 2.12-1.96 (m, 1H), 1.89-1.84 (m, 2H).

### Examples 21 and 22

### 4-[[1-[2-[(1R)-1-(2,2-difluoro-1,3-benzodioxol-5-yl)ethoxy]-4-pyridyl]-3-(trifluoromethyl)-4,5,6,7-tetrahydroindazol-7-yl]oxy]benzoic acid

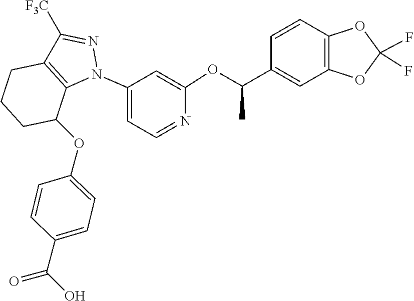 [SMILES: C[C@@H](Oc1cc(-n2nc(C(F)(F)F)c3c2C(Oc2ccc(C(=O)O)cc2)CCC3)ccn1)c1ccc2c(c1)OC(F)(F)O2]

### 4-[[1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]benzoic acid

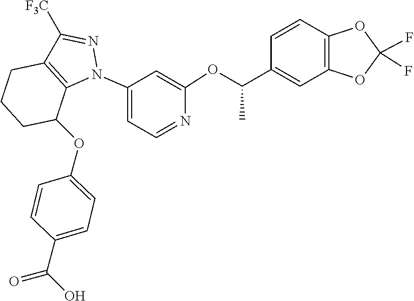 [SMILES: C[C@H](Oc1cc(-n2nc(C(F)(F)F)c3c2C(Oc2ccc(C(=O)O)cc2)CCC3)ccn1)c1ccc2c(c1)OC(F)(F)O2]

### Step 1. Synthesis of 1-(2-(1-(2,2-difluorobenzo[d][1,3]dioxol-5-yl)ethoxy)pyridine-4-yl)-3-(trifluoromethyl)-1,4,5,6-tetrahydro-7H-indazol-7-one

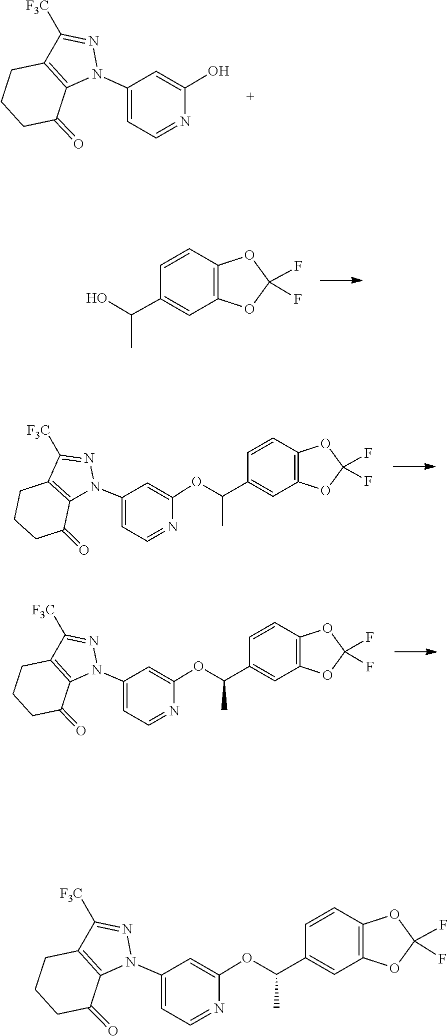 [SMILES: O=C1CCCc2c(C(F)(F)F)nn(-c3ccnc(O)c3)c21 ; CC(O)c1ccc2c(c1)OC(F)(F)O2 ; CC(Oc1cc(-n2nc(C(F)(F)F)c3c2C(=O)CCC3)ccn1)c1ccc2c(c1)OC(F)(F)O2 ; C[C@@H](Oc1cc(-n2nc(C(F)(F)F)c3c2C(=O)CCC3)ccn1)c1ccc2c(c1)OC(F)(F)O2 ; C[C@H](Oc1cc(-n2nc(C(F)(F)F)c3c2C(=O)CCC3)ccn1)c1ccc2c(c1)OC(F)(F)O2]

To the solution of 1-(2-hydroxy-4-pyridyl)-3-(trifluoromethyl)-5,6-dihydro-4H-indazol-7-one (Intermediate D-4) (4000 mg, 13.5 mmol) in THF (50 mL) were added 1-(2,2-difluoro-1,3-benzodioxol-5-yl)ethanol (Intermediate C-1) (3265 mg, 16.1 mmol), and triphenylphosphine (4236 mg, 16.1 mmol). After the solution was cooled to 0° C., diethyl azodicarboxylate (2.6 mL, 16.1 mmol) was added dropwise at 0° C. and then stirred at 25° C. for 1 h. The solution was concentrated, and the crude product was then purified by flash column chromatography eluted with 10% ethyl acetate in isohexane to get the racemic compound (4500 mg, 69% yield) which was purified by SFC to obtain two enantiomers of the title compound with the first fractions designated as P1 (2100 mg, 47% yield) and the second designated as P2 (2000 mg, 44% yield), respectively.

LC-MS (Method 2): Retention time=2.36 min. MS (ESI) m/z 482.0 (M+H)⁺.

### Step 2a. Synthesis of 1-(2-((R)-1-(2,2-difluorobenzo[d][1,3]dioxol-5-yl)ethoxy)pyridine-4-yl)-3-(trifluoromethyl)-4,5,6,7-tetrahydro-1H-indazol-7-ol

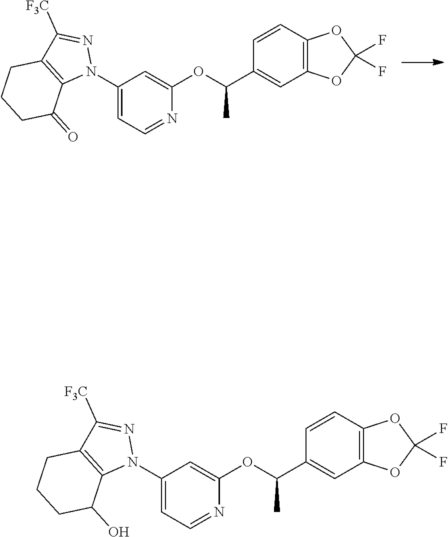 [SMILES: C[C@@H](Oc1cc(-n2nc(C(F)(F)F)c3c2C(=O)CCC3)ccn1)c1ccc2c(c1)OC(F)(F)O2 ; C[C@@H](Oc1cc(-n2nc(C(F)(F)F)c3c2C(O)CCC3)ccn1)c1ccc2c(c1)OC(F)(F)O2]

To a solution of 1-[2-[(1R)-1-(2,2-difluoro-1,3-benzodioxol-5-yl)ethoxy]-4-pyridyl]-3-(trifluoromethyl)-5,6-dihydro-4H-indazol-7-one (the intermediate P1 in Step 1) (400 mg, 0.831 mmol) in methanol (10 mL) was added sodium borohydride (126 mg, 3.32 mmol). The solution was then stirred at rt for 2 h under Ar. The reaction was quenched with water. The mixture was then diluted with water (50 mL) and extracted with EA (3×50 mL). The organic layer was washed with brine (20 mL) and dried over Na₂SO₄, filtered, and concentrated. The crude product was purified by flash chromatography (Biotage, 40 g silica gel column @ 60 mL/min, eluted with 0-30% ethyl acetate in petroleum ether for 20 min) to get the title compound (360 mg, 83% yield) as a light-yellow oil.

LC-MS (Method 1): Retention time=2.38 min. MS (ESI) m/z 484.3 (M+H)⁺.

### Step 2b. Synthesis of 1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-ol

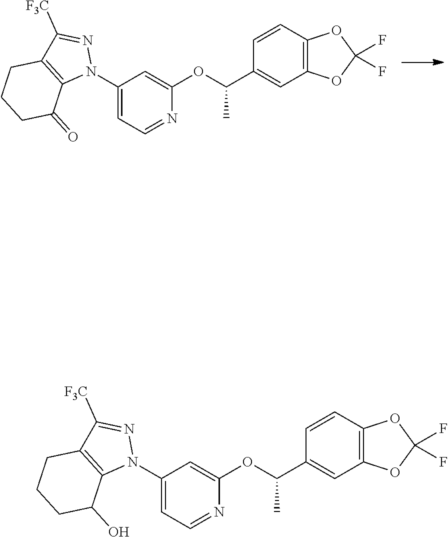 [SMILES: C[C@H](Oc1cc(-n2nc(C(F)(F)F)c3c2C(=O)CCC3)ccn1)c1ccc2c(c1)OC(F)(F)O2 ; C[C@H](Oc1cc(-n2nc(C(F)(F)F)c3c2C(O)CCC3)ccn1)c1ccc2c(c1)OC(F)(F)O2]

The title mixture of diastereomers were obtained (350 mg, 83% yield) as a light-yellow oil by the same protocol as described in Step 2a using the starting material of 1-[2-[(1S)-1-(2,2-difluoro-1,3-benzodioxol-5-yl)ethoxy]-4-pyridyl]-3-(trifluoromethyl)-5,6-dihydro-4H-indazol-7-one (the intermediate P2 in Step 1).

LC-MS (Method 1): Retention time=2.38 min. MS (ESI) m/z 484.3 (M+H)⁺.

### Step 3a. Synthesis of methyl 4-((1-(2-((R)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoate

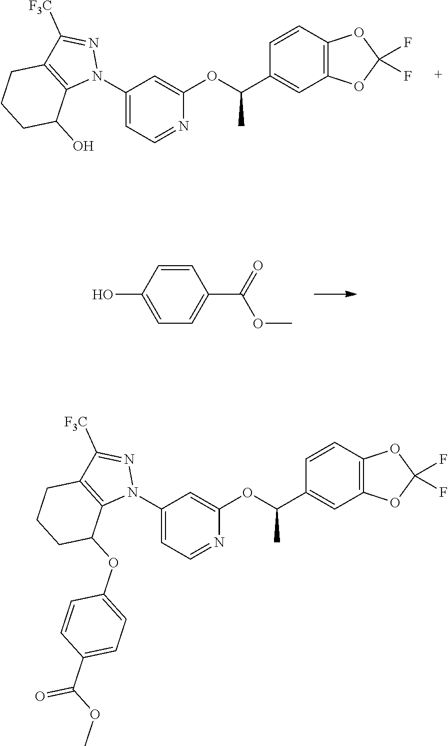 [SMILES: C[C@@H](Oc1cc(-n2nc(C(F)(F)F)c3c2C(O)CCC3)ccn1)c1ccc2c(c1)OC(F)(F)O2 ; COC(=O)c1ccc(O)cc1 ; COC(=O)c1ccc(OC2CCCc3c(C(F)(F)F)nn(-c4ccnc(O[C@H](C)c5ccc6c(c5)OC(F)(F)O6)c4)c32)cc1]

To a solution of 1-[2-[(1R)-1-(2,2-difluoro-1,3-benzodioxol-5-yl)ethoxy]-4-pyridyl]-3-(trifluoromethyl)-4,5,6,7-tetrahydroindazol-7-ol (the intermediate from Step 2a) (100 mg, 0.207 mmol) in THF (3 mL) was added methyl 4-hydroxybenzoate (38 mg, 0.248 mmol) and triphenylphosphine (71 mg, 0.269 mmol). The solution was then cooled to 0° C. and DEAD (0.049 mL, 0.310 mmol) was added dropwise under N₂. The solution was stirred at 25° C. for 1-2 h. The reaction solution was then diluted with an aqueous NH₄Cl solution. The solution was extracted with EA (3×15 mL). The organic layers were combined and dried over MgSO₄ and concentrated in vacuo. The resulting residue was purified by silica gel chromatography (EA:PE=1:8) to give the title mixture of diastereomers (50 mg, 16% yield) as a white solid.

LC-MS (Method 2): Retention time=2.65 min. MS (ESI) m/z 618.2 (M+H)⁺.

### Step 3b. Synthesis of methyl 4-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoate

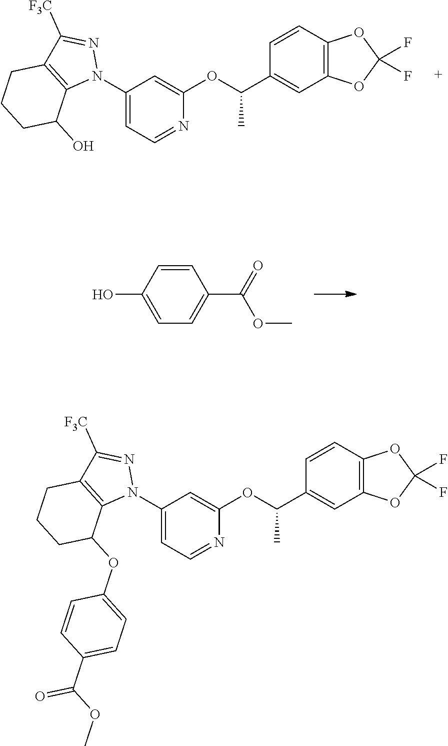 [SMILES: C[C@H](Oc1cc(-n2nc(C(F)(F)F)c3c2C(O)CCC3)ccn1)c1ccc2c(c1)OC(F)(F)O2 ; COC(=O)c1ccc(O)cc1 ; COC(=O)c1ccc(OC2CCCc3c(C(F)(F)F)nn(-c4ccnc(O[C@@H](C)c5ccc6c(c5)OC(F)(F)O6)c4)c32)cc1]

The title mixture of diastereomers were obtained (60 mg, 23% yield) as a white solid by the same protocol as described in Step 3a using the starting material of 1-[2-[(1S)-1-(2,2-difluoro-1,3-benzodioxol-5-yl)ethoxy]-4-pyridyl]-3-(trifluoromethyl)-4,5,6,7-tetrahydroindazol-7-ol (the intermediate from Step 2b).

LC-MS (Method 2): Retention time=2.65 min. MS (ESI) m/z 618.2 (M+H)⁺.

### Step 4a. Synthesis of 4-((1-(2-((R)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoic acid

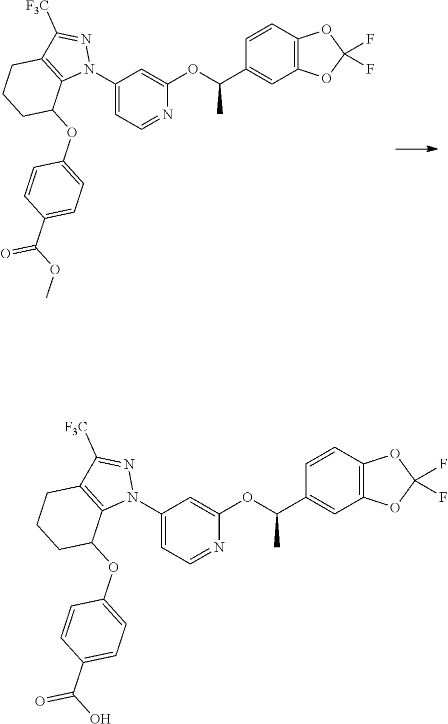 [SMILES: COC(=O)c1ccc(OC2CCCc3c(C(F)(F)F)nn(-c4ccnc(O[C@H](C)c5ccc6c(c5)OC(F)(F)O6)c4)c32)cc1 ; C[C@@H](Oc1cc(-n2nc(C(F)(F)F)c3c2C(Oc2ccc(C(=O)O)cc2)CCC3)ccn1)c1ccc2c(c1)OC(F)(F)O2]

To a solution of methyl 4-[[1-[3-[(1R)-1-(2,2,6-trifluoro-1,3-benzodioxol-5-yl)ethoxy]phenyl]-3-(trifluoromethyl)-4,5,6,7-tetrahydroindazol-7-yl]oxy]benzoate (the intermediate from Step 3a) (50 mg, 0.0946 mmol) in methanol (2 mL), THF (2 mL) and water (1 mL) was added LiOH (9.1 mg, 0.378 mmol). After the solution was stirred at 25° C. for 12 h, the solution was concentrated under vacuum and purified by prep-HPLC to get the title compound (28 mg, 48% yield) as a white solid.

LC-MS (Method 1): Retention time=2.38 min. MS (ESI) m/z 604.2 (M+H)⁺.

### Step 4b. Synthesis of 4-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoic acid

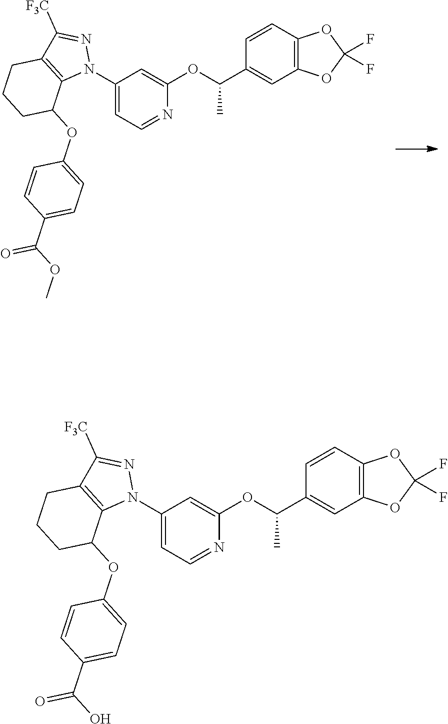 [SMILES: COC(=O)c1ccc(OC2CCCc3c(C(F)(F)F)nn(-c4ccnc(O[C@@H](C)c5ccc6c(c5)OC(F)(F)O6)c4)c32)cc1 ; C[C@H](Oc1cc(-n2nc(C(F)(F)F)c3c2C(Oc2ccc(C(=O)O)cc2)CCC3)ccn1)c1ccc2c(c1)OC(F)(F)O2]

The title mixture of diastereomers were obtained (34 mg, 69% yield) as a white solid by the same protocol as described in Step 2a using the starting material of methyl 4-[[1-[3-[(1S)-1-(2,2,6-trifluoro-1,3-benzodioxol-5-yl)ethoxy]phenyl]-3-(trifluoromethyl)-4,5,6,7-tetrahydroindazol-7-yl]oxy]benzoate (the intermediate from Step 3b).

LC-MS (Method 1): Retention time=1.62 min. MS (ESI) m/z 604.1 (M+H)⁺.

### Examples 23 and 24

### 4-[[(7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]benzoic acid

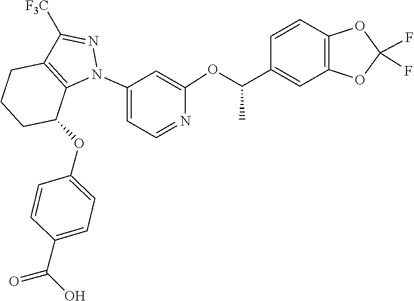 [SMILES: C[C@H](Oc1cc(-n2nc(C(F)(F)F)c3c2[C@H](Oc2ccc(C(=O)O)cc2)CCC3)ccn1)c1ccc2c(c1)OC(F)(F)O2]

### 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]benzoic acid

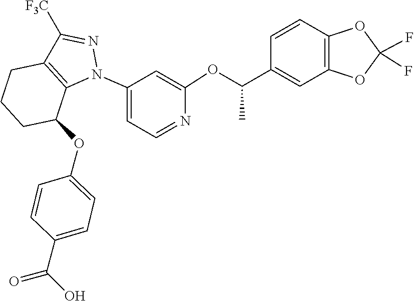 [SMILES: C[C@H](Oc1cc(-n2nc(C(F)(F)F)c3c2[C@@H](Oc2ccc(C(=O)O)cc2)CCC3)ccn1)c1ccc2c(c1)OC(F)(F)O2]

Synthetic method 1.

### Step 1. Synthesis of methyl 4-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoate

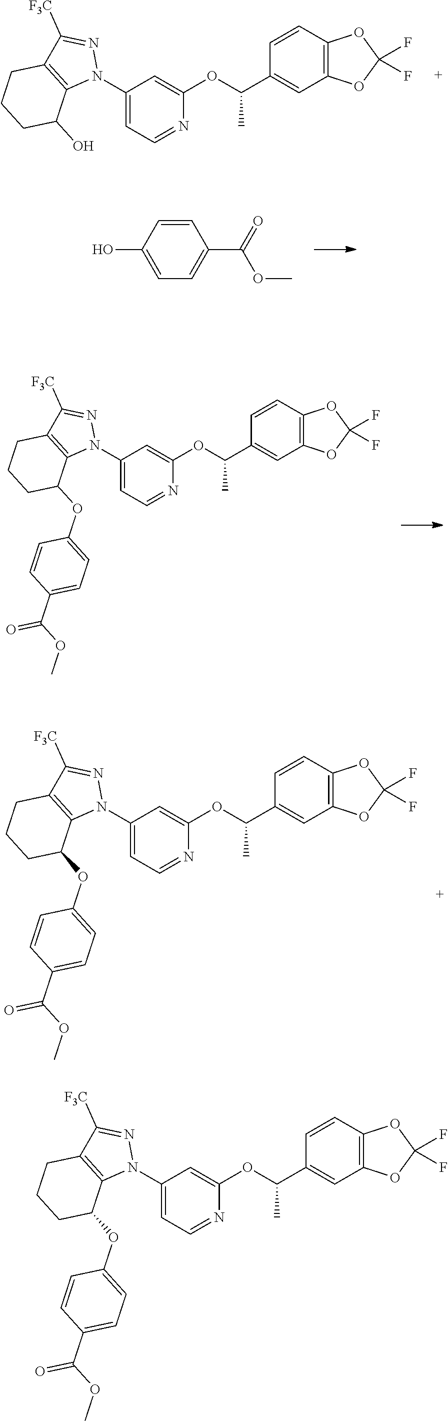 [SMILES: C[C@H](Oc1cc(-n2nc(C(F)(F)F)c3c2C(O)CCC3)ccn1)c1ccc2c(c1)OC(F)(F)O2 ; COC(=O)c1ccc(O)cc1 ; COC(=O)c1ccc(OC2CCCc3c(C(F)(F)F)nn(-c4ccnc(O[C@@H](C)c5ccc6c(c5)OC(F)(F)O6)c4)c32)cc1 ; COC(=O)c1ccc(O[C@H]2CCCc3c(C(F)(F)F)nn(-c4ccnc(O[C@@H](C)c5ccc6c(c5)OC(F)(F)O6)c4)c32)cc1 ; COC(=O)c1ccc(O[C@@H]2CCCc3c(C(F)(F)F)nn(-c4ccnc(O[C@@H](C)c5ccc6c(c5)OC(F)(F)O6)c4)c32)cc1]

To the solution of 1-[2-[(1S)-1-(2,2-difluoro-1,3-benzodioxol-5-yl)ethoxy]-4-pyridyl]-3-(trifluoromethyl)-4,5,6,7-tetrahydroindazol-7-ol (Intermediate from Examples 21 and 22, Step 2b) (800 mg, 1.65 mmol) in THF (10 mL) were added methyl 4-hydroxybenzoate (302 mg, 1.99 mmol) and PPh₃ (521 mg, 1.99 mmol). After the solution was stirred at 0° C. for 5 min, DEAD (0.31 mL, 1.99 mmol) was added dropwise at 0° C. The solution was then stirred at 25° C. for 1 h. The solution was concentrated, and the crude product was then purified by flash column chromatography eluted with 10% ethyl acetate in isohexane to get the mixture of diastereomers (650 mg, 69% yield) which was further separated by SFC to get the title compounds with the first fraction designated as P1 (300 mg, 47%; a yellow oil), and the second fractions as P2 (300 mg, 44%; a yellow oil).

LC-MS (Method 1): Retention time=2.65 min. MS (ESI) m/z 618.2 [M+H]⁺.

### Step 2a. Synthesis of 4-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoic acid

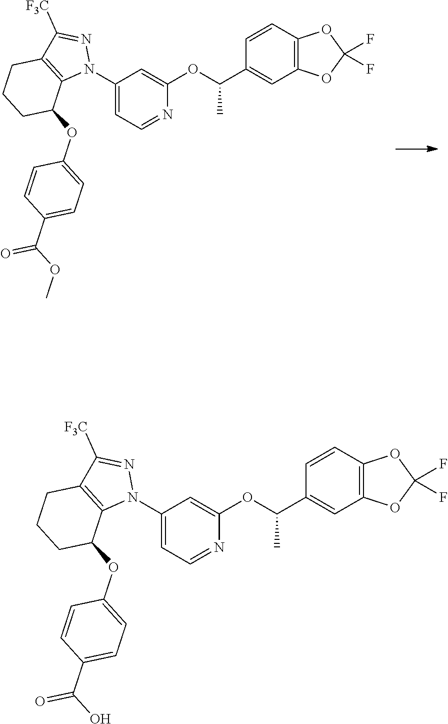 [SMILES: COC(=O)c1ccc(O[C@H]2CCCc3c(C(F)(F)F)nn(-c4ccnc(O[C@@H](C)c5ccc6c(c5)OC(F)(F)O6)c4)c32)cc1 ; C[C@H](Oc1cc(-n2nc(C(F)(F)F)c3c2[C@@H](Oc2ccc(C(=O)O)cc2)CCC3)ccn1)c1ccc2c(c1)OC(F)(F)O2]

To the solution of methyl 4-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoate (P1 of the intermediate from Step 1 above) (300 mg, 0.486 mmol) in methanol (10 mL), THF (10 mL) and water (5 mL) was added lithium hydroxide (47 mg, 1.94 mmol). The solution was then stirred at 25° C. overnight. The solution was concentrated under vacuum and purified by prep-HPLC to get the title compound (185 mg, 48% yield) as white solid.

LC-MS (Method 1): Retention time=1.86 min. MS (ESI) m/z 602.0 [M−H]⁺.

¹H NMR (500 MHZ, CH₃OH-d₄) δ 8.06 (d, J=7.5 Hz, 1H), 8.03 (d, J=11 Hz, 2H), 7.16 (dd, J=7.0, 2.5 Hz, 1H), 7.12 (dd, J=7.0, 5.0 Hz, 2H), 7.05 (d, J=1.7 Hz, 1H), 7.03-6.87 (m, 3H), 6.03 (q, J=8.0 Hz, 1H), 5.74 (t, J=4.5 Hz, 1H), 2.87 (d, J=21 Hz, 1H), 2.63 (ddd, J=16.7, 11.0, 5.7 Hz, 1H), 2.29 (d, J=17 Hz, 1H), 2.00 (dd, J=12.2, 6.9 Hz, 1H), 1.95-1.75 (m, 2H), 1.43 (d, J=8.0 Hz, 3H).

### Step 2b. Synthesis of 4-(((R)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoic acid

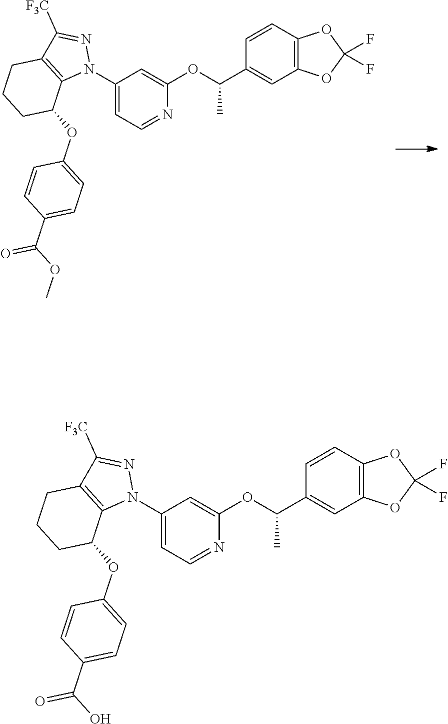 [SMILES: COC(=O)c1ccc(O[C@@H]2CCCc3c(C(F)(F)F)nn(-c4ccnc(O[C@@H](C)c5ccc6c(c5)OC(F)(F)O6)c4)c32)cc1 ; C[C@H](Oc1cc(-n2nc(C(F)(F)F)c3c2[C@H](Oc2ccc(C(=O)O)cc2)CCC3)ccn1)c1ccc2c(c1)OC(F)(F)O2]

The title compound was obtained (158 mg, 54% yield) as a white solid by the same protocol as described in Step 2a using the starting material of methyl 4-(((R)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoate (P2 of the intermediate from Step 1 above). LCMS (Method 1): Retention time=1.71 min. MS (ESI−) m/z 602 [M−H]⁻.

¹H NMR (400 MHZ, CD₃OD) δ 7.93 (dd, J=20.9, 8.8 Hz, 2H), 7.30-6.81 (m, 9H), 5.51 (m, 1H), 5.15 (ddd, J=26.7, 12.5, 6.2 Hz, 1H), 2.86 (d, J=16.3 Hz, 1H), 2.69-2.53 (m, 1H), 2.27 (d, J=14.3 Hz, 1H), 2.09-1.70 (m, 3H), 1.42 (dd, J=21.4, 6.3 Hz, 3H).

### Example 24, Synthetic Method 2

### Step 1. Synthesis of 4-bromo-2-(1-phenylethoxy)pyridine

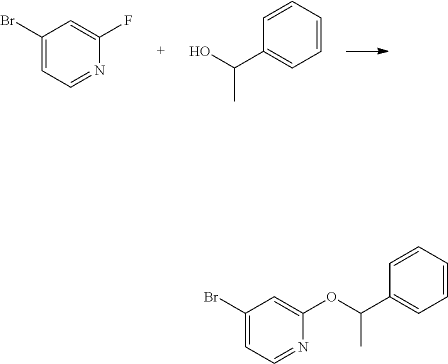

To a solution of 4-bromo-2-fluoro-pyridine (20.0 g, 114 mmol) and 1-phenylethanol (13.9 g, 114 mmol) in DMF (200 mL) was added Cs₂CO₃ (111 g, 342 mmol). The reaction was stirred at 100° C. overnight. The mixture was poured into water (2 L) and extracted with EA (200 mL×3). The organic layers were combined, washed with brine, dried over Na₂SO₄, filtered, and concentrated. The crude was then purified by flash column chromatography eluting with 10% ethyl acetate in PE to get the title compound (29.0 g, 91.8%) as a yellow solid.

LCMS (Method 1): Retention time=2.45 min. MS (ESI) m/z 174.1, 176.1 [M-103]⁺.

### Step 2. Synthesis of tert-butyl 1-(2-(1-phenylethoxy)pyridine-4-yl) hydrazinecarboxylate

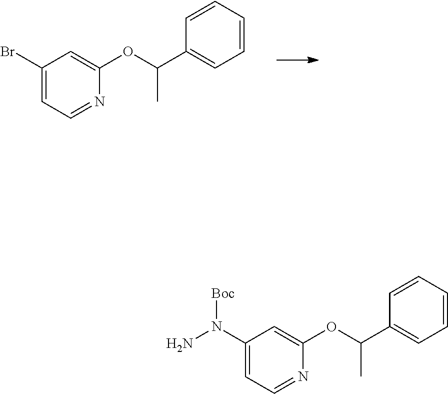 [SMILES: CC(Oc1cc(Br)ccn1)c1ccccc1 ; CC(Oc1cc(N(N)C(=O)OC(C)(C)C)ccn1)c1ccccc1]

To the solution of 4-bromo-2-(1-phenylethoxy)pyridine (15.0 g, 53.9 mmol) in 1,4-dioxane (150 mL) were added tert-butyl N-amino carbamate (8.6 g, 64.7 mmol), XantPhos (3.1 g, 5.39 mmol), tris(dibenzylideneacetone) dipalladium (4.9 g, 5.39 mmol), and Cs₂CO₃ (52.8 g, 162 mmol). Then the mixture was stirred at 100° C. overnight under Ar. The mixture was poured into water (2 L) and extracted with EA (200 mL×3). The organic layers were combined, washed with brine, dried over Na₂SO₄, filtered, and concentrated. The crude was then purified by flash column chromatography eluting with 10% ethyl acetate in PE to get the title compound (16.0 mg, 90.1%) as a yellow solid.

LCMS (Method 1): Retention time=2.11 min. MS (ESI) m/z 226 [M-103]⁺.

### Step 3. Synthesis of 1-(2-hydroxypyridin-4-yl)-3-(trifluoromethyl)-5,6-dihydro-1H-indazol-7(4H)-one

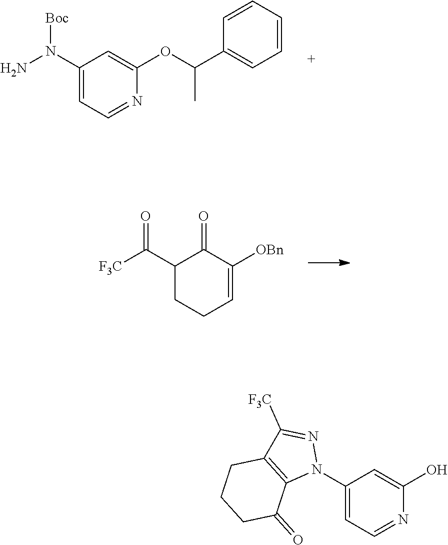 [SMILES: CC(Oc1cc(N(N)C(=O)OC(C)(C)C)ccn1)c1ccccc1 ; O=C1C(OCc2ccccc2)=CCCC1C(=O)C(F)(F)F ; O=C1CCCc2c(C(F)(F)F)nn(-c3ccnc(O)c3)c21]

To a solution of tert-butyl N-amino-N-[2-(1-phenylethoxy)-4-pyridyl]carbamate (10.0 g, 30.4 mmol) in 2,2,2-trifluoroethanol (100 mL) was added sulfuric acid (25 mL). Then the mixture was stirred at 80° C. overnight. The mixture was poured into water (500 mL) and adjusted pH to 8-9 with NaHCO₃, then extracted with EA (50 mL×3). The organic layers were combined, washed with brine, dried over Na₂SO₄, filtered, and concentrated. The crude was then purified by flash column chromatography eluting with 10% ethyl acetate in PE to get the title compound (5.0 g, 55.4%) as a yellow solid.

LCMS (Method 1): Retention time=1.72 min. MS (ESI) m/z 298.3 [M+H]⁺.

### Step 4. Synthesis of(S)-1-(2-(1-(2,2-difluorobenzo[d][1,3]dioxol-5-yl)ethoxy)pyridine-4-yl)-3-(trifluoromethyl)-5,6-dihydro-1H-indazol-7 (4H)-one

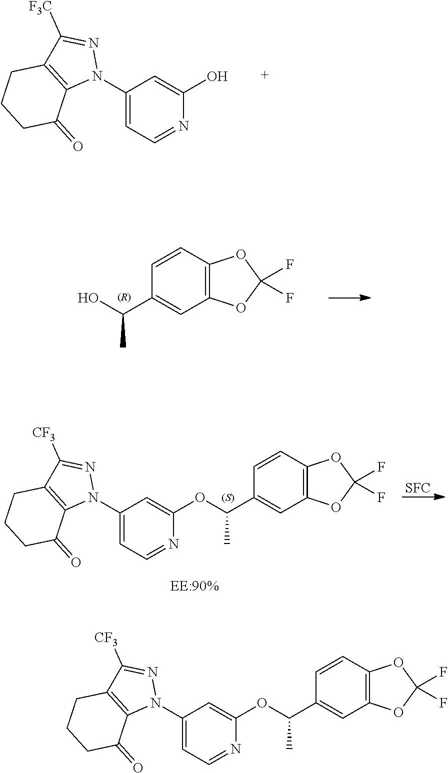

To a solution of 1-(2-hydroxy-4-pyridyl)-3-(trifluoromethyl)-5,6-dihydro-4H-indazol-7-one (4000 mg, 13.5 mmol) in THF (50 mL) were added R-1-(2,2-difluorobenzo[d][1,3]dioxol-5-yl)ethan-1-ol (Intermediate C-1R) (3265 mg, 16.1 mmol), and PPh₃ (4236 mg, 16.1 mmol). After the mixture was cooled to 0° C., DEAD (2.6 mL, 16.1 mmol) was added dropwise at 0° C. and then stirred at rt for 1 h under N₂. The mixture was concentrated, and the crude was then purified by flash column chromatography eluting with 10% ethyl acetate in PE to get the crude compound (4.5 g, ee 90%). This crude compound was purified further by SFC to afford the title compound (3.2 g, 49%, ee >99%) as a white solid.

LCMS (Method 2): Retention time=2.36 min. MS (ESI) m/z 298.0 (M-183)⁺.

### Step 5. Synthesis of (7R)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-ol

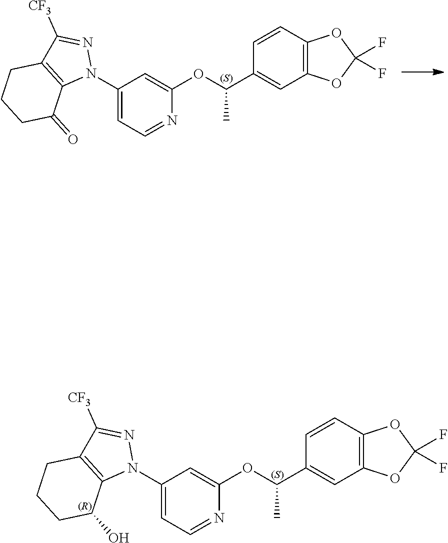

In a 200 mL reactor, the product from Step 4 (5.0 g, 10.4 mmol) was suspended in IPA (yellow suspension) (10 vol) at room temperature. TEA (0.6 vol) and catalyst (R,R)-TsDPEN-RuCl (p-cymene) (0.006 wt) were added to the mixture, and 3 cycles of vacuum/nitrogen exchange were applied. Formic acid (0.4 vol) was added slowly to the mixture in 10 min. (exothermic reaction from 20 to 25° C.). Temperature was increased to 45° C. and the solution was stirred for 4 h. Reaction mixture was concentrated under vacuum. Water (10 vol) was added and adjusted pH to 7-8 with 5% NaHCO₃ aqueous solution, then extracted with EA (2 vol×3). The organic layers were combined, washed with brine, dried over Na₂SO₄, filtered and concentrated. The crude was purified by flash column chromatography eluting with 20% ethyl acetate in Isohexane to get the title compound (3.0 g, 60%) as a yellow solid.

LCMS (Method 1): Retention time=1.73 min. MS (ESI) m/z 300 [M−183]⁺.

### Step 6. Synthesis of methyl 4-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoate

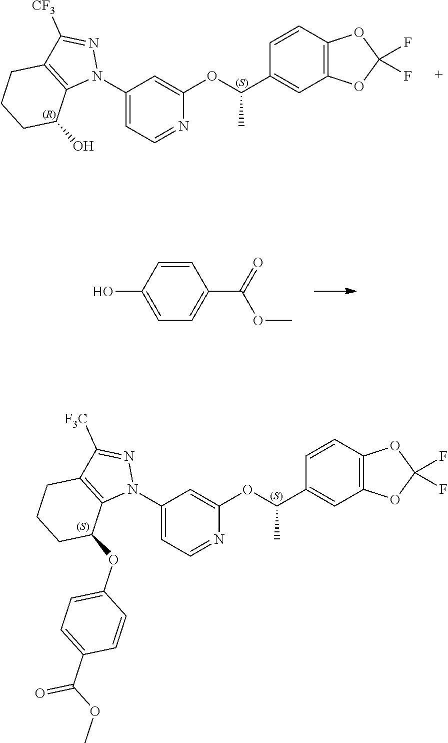

To a solution of R-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-ol (3000 mg, 6.2 mmol) in THF (10 mL) were added methyl 4-hydroxybenzoate (1.1 g, 7.4 mmol) and PPh₃ (2.4 g, 9.3 mmol). The mixture was cooled to 0° C. and DEAD (1.6 g, 9.3 mmol) was added dropwise at 0° C. under N₂. Then the mixture was stirred at rt for 1 h. The mixture was concentrated, and the crude was then purified by flash column chromatography eluting with 10% ethyl acetate in PE to get the title compound (3.0 g, 79%) as a white solid.

LCMS (Method 1): Retention time=2.65 min. MS (ESI) m/z 434.2 [M-183]⁺.

### Step 7. Synthesis of 4-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)benzoic acid

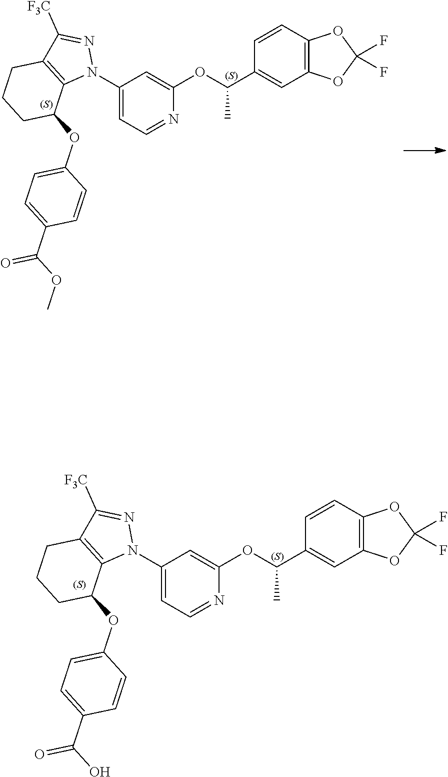

To a solution of methyl 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]benzoate (3.0 g, 4.86 mmol) in methanol (20 mL), THF (20 mL) and water (10 mL) was added lithium hydroxide (465 mg, 19.4 mmol). The reaction was stirred at 25° C. overnight. Then the mixture was concentrated, and the crude was purified by pre-HPLC (NH₄HCO₃) to get the title compound (2204 mg, 75.2%) as a white solid.

The analytics (LCMS and 1HNMR) are identical as the sample obtained through the synthetic Method 1.

### Examples 25 and 26

### 4-[[(7S)-1-[3-[(1S)-1-(2,2-difluoro-1,3-benzodioxol-5-yl)ethoxy]phenyl]-3-(trifluoromethyl)-4,5,6,7-tetrahydroindazol-7-yl]oxy]benzoic acid

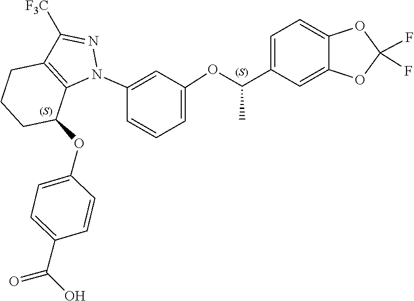

### 4-[[(7R)-1-[3-[(1S)-1-(2,2-difluoro-1,3-benzodioxol-5-yl)ethoxy]phenyl]-3-(trifluoromethyl)-4,5,6,7-tetrahydroindazol-7-yl]oxy]benzoic acid

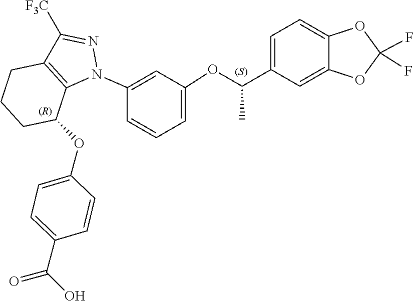

The title compound was synthesized essentially in the same manner as Examples 23 and 24.

P1:115.5 mg, 70%, a white solid.

LC-MS (Method 2): Retention time=1.63 min. MS (ESI) m/z 603.2 [M+H]⁺.

¹H NMR (500 MHZ, CH₃OH-d₄) δ 7.98 (d, J=8.7 Hz, 2H), 7.22-7.18 (m, 1H), 7.12-6.90 (m, 7H), 6.86 (d, J=8.5 Hz, 1H), 5.49 (s, 1H), 5.17 (q, J=6.3 Hz, 1H), 2.89-2.83 (m, 1H), 2.65-2.58 (m, 1H), 2.27-2.22 (m, 1H), 2.01-1.89 (m, 3H), 1.41 (d, J=6.3 Hz, 3H).

P2:105 mg, 67% yield, a white solid.

LC-MS (Method 2): Retention time=1.63 min. MS (ESI) m/z 603.2 [M+H]⁺.

¹H NMR (500 MHZ, CH₃OH-d₄) δ 7.97 (d, J=8.7 Hz, 2H), 7.22-7.18 (m, 1H), 7.12-6.90 (m, 7H), 6.86 (d, J=8.4 Hz, 1H), 5.49 (s, 1H), 5.17 (q, J=6.3 Hz, 1H), 2.89-2.83 (m, 1H), 2.67-2.50 (m, 1H), 2.27-2.22 (m, 1H), 2.01-1.89 (m, 3H), 1.41 (d, J=6.3 Hz, 3H).

### Example 27

### 4-[[(7S)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]oxy]benzoic acid

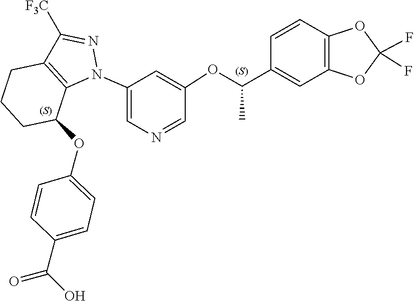

The title compound was synthesized essentially in the same manner as Examples 23 and 24.

Chiral Method: Column Name: OX—H 4.6×100 mm 5 μm. Solvent: EtOH [0.2% NH₃ (7M in MeOH)]; Retention time=2.39 min; ee=100%.

LC-MS (Method 4): Retention time=1.85 min. MS (ESI) m/z 604.1 [M+H]⁺.

¹H NMR (500 MHz, CH₃OH-d₄) δ 8.31 (d, J=2.0 Hz, 1H), 8.19 (d, J=2.5 Hz, 1H), 7.99 (d, J=8.7 Hz, 2H), 7.52 (t, J=2.3 Hz, 1H), 7.11 (dd, J=14.0, 4.9 Hz, 2H), 6.97 (dd, J=9.4, 2.2 Hz, 3H), 5.60 (t, J=3.1 Hz, 1H), 5.19 (q, J=6.3 Hz, 1H), 2.87 (d, J=16.0 Hz, 1H), 2.62 (ddd, J=16.7, 10.9, 5.8 Hz, 1H), 2.27 (d, J=14.2 Hz, 1H), 2.05-1.92 (m 3H), 1.44 (d, J=6.3 Hz, 3H)

### Examples 28 and 29

### 4-((1-(3-((R)-1-(2,2-difluorobenzo[d][1,3]dioxol-5-yl)ethoxy)phenyl)-3-(trifluoromethyl)-4,5,6,7-tetrahydro-1H-indazol-7-yl)oxy)benzoic acid

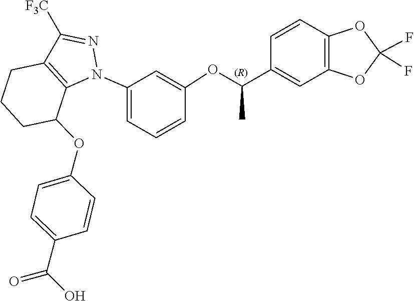

### 4-((1-(3-((S)-1-(2,2-difluorobenzo[d][1,3]dioxol-5-yl)ethoxy)phenyl)-3-(trifluoromethyl)-4,5,6,7-tetrahydro-1H-indazol-7-yl)oxy)benzoic acid

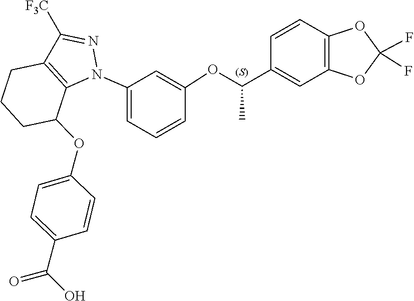

The title mixture of diastereomers were synthesized essentially in the same manner as Examples 21 and 22.

P1: LC-MS (Method 1): Retention time=2.33 min. MS (ESI) m/z 603.3 [M+H]⁺.

¹H NMR (400 MHZ, CH₃OH-d₄) δ 7.98 (d, J=8.8 Hz, 1H), 7.92 (s, J=8.8 HZ, 1H), 7.23-7.18 (m, 1H), 7.15-6.84 (m, 8H), 5.66 (s, 0.5H), 5.50 (d, J=2.8 Hz, 0.5H), 5.22-5.17 (m, 1H), 2.89-2.84 (m, 1H), 2.63-2.62 (m, 1H), 2.26-2.22 (m, 1H), 2.04-1.77 (m, 3H), 1.44-1.39 (m, 3H) ppm.

P2: LC-MS (Method 1): Retention time=2.33 min. MS (ESI) m/z 603.3 [M+H]⁺.

¹H NMR (400 MHZ, CH₃OH-d₄) δ 8.09-7.87 (m, 2H), 7.23-7.18 (m, 1H), 7.14-6.84 (m, 8H), 5.66 (s, 0.5H), 5.51 (d, J=2.8 Hz, 0.5H), 5.23-5.18 (m, 1H), 2.89-2.83 (m, 1H), 2.70-2.53 (m, 1H), 2.36-2.17 (m, 1H), 2.11-1.78 (m, 3H), 1.46-1.40 (m, 3H).

### Examples 30 and 31

### 4-((1-(3-((R)-1-(2,2-difluoro-[1,3]dioxolo[4,5-c]pyridine-6-yl)ethoxy)phenyl)-3-(trifluoromethyl)-4,5,6,7-tetrahydro-1H-indazol-7-yl)oxy)benzoic acid

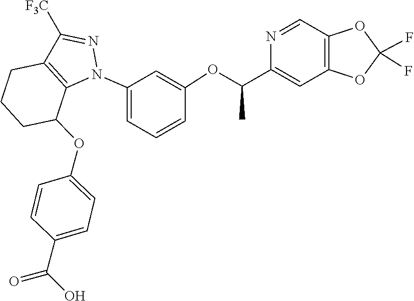 [SMILES: C[C@@H](Oc1cccc(-n2nc(C(F)(F)F)c3c2C(Oc2ccc(C(=O)O)cc2)CCC3)c1)c1cc2c(cn1)OC(F)(F)O2]

### 4-((1-(3-((S)-1-(2,2-difluoro-[1,3]dioxolo[4,5-c]pyridine-6-yl)ethoxy)phenyl)-3-(trifluoromethyl)-4,5,6,7-tetrahydro-1H-indazol-7-yl)oxy)benzoic acid

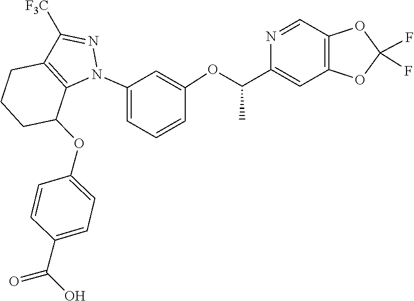 [SMILES: C[C@H](Oc1cccc(-n2nc(C(F)(F)F)c3c2C(Oc2ccc(C(=O)O)cc2)CCC3)c1)c1cc2c(cn1)OC(F)(F)O2]

The title mixture of diastereomers was synthesized essentially in the same manner as Examples 21 and 22.

P1: LC-MS (Method 2): Retention time=1.53 min. MS (ESI) m/z 604.2 [M+H]⁺.

¹H NMR (500 MHZ, CH₃OH-d₄) δ 8.36 (d, J=3.7 Hz, 1H), 7.95 (d, J=8.8 Hz, 1H), 7.89 (d, J=8.7 Hz, 1H), 7.34-7.03 (m, 4H), 6.97 (d, J=8.4 Hz, 1H), 6.93-6.81 (m, 2H), 5.67 (s, 0.5H), 5.54 (s, 0.5H), 5.26-5.20 (m, 1H), 2.90-2.80 (m, 1H), 2.69-2.57 (m, 1H), 2.26-2.20 (m, 1H), 2.08-1.82 (m, 3H), 1.50-1.45 (m, 3H) (mixture of two diastereomers).

P2: LC-MS (Method 2): Retention time=1.53 min. MS (ESI) m/z 604.2 [M+H]⁺.

¹H NMR (500 MHZ, CH₃OH-d₄) δ 8.36 (d, J=3.6 Hz, 1H), 7.95 (d, J=8.8 Hz, 1H), 7.89 (d, J=8.8 Hz, 1H), 7.31-7.02 (m, 4H), 6.97 (d, J=8.8 Hz, 1H), 6.93-6.80 (m, 2H), 5.67 (s, 0.5H), 5.54 (d, J=2.4 Hz, 0.5H), 5.26-5.21 (m, 1H), 2.89-2.82 (m, 1H), 2.69-2.58 (m, 1H), 2.27-2.20 (m, 1H), 2.02-1.86 (m, 3H), 1.50-1.45 (m, 3H) ppm. (Mixture of two diastereomers)

### Examples 32 and 33

### 4-((1-(3-((R)-1-(2,2,6-trifluorobenzo[d][1,3]dioxol-5-yl)ethoxy)phenyl)-3-(trifluoromethyl)-4,5,6,7-tetrahydro-1H-indazol-7-yl)oxy)benzoic acid

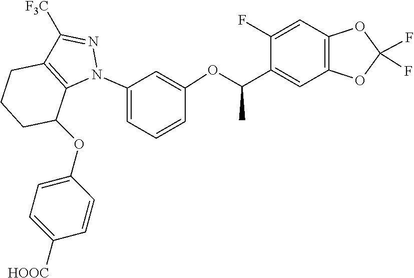 [SMILES: C[C@@H](Oc1cccc(-n2nc(C(F)(F)F)c3c2C(Oc2ccc(C(=O)O)cc2)CCC3)c1)c1cc2c(cc1F)OC(F)(F)O2]

### 4-((1-(3-((S)-1-(2,2,6-trifluorobenzo[d][1,3]dioxol-5-yl)ethoxy)phenyl)-3-(trifluoromethyl)-4,5,6,7-tetrahydro-1H-indazol-7-yl)oxy)benzoic acid

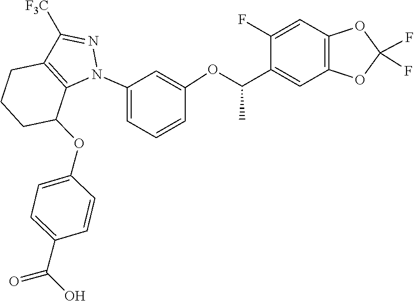 [SMILES: C[C@H](Oc1cccc(-n2nc(C(F)(F)F)c3c2C(Oc2ccc(C(=O)O)cc2)CCC3)c1)c1cc2c(cc1F)OC(F)(F)O2]

The title compound was synthesized essentially in the same manner as Examples 21 and 22.

P1: LC-MS (Method 1): Retention time=2.38 min. MS (ESI) m/z 621.2 [M+H]⁺.

¹H NMR (500 MHZ, CH₃OH-d₄) δ 7.92 (m, 8.7 Hz, 2H), 7.26-6.90 (m, 6H), 6.86 (dd, J=11.8, 4.5 Hz, 2H), 5.71-5.24 (m, 2H), 2.87 (d, J=16.8 Hz, 1H), 2.63 (d, J=6.1 Hz, 1H), 2.25 (t, J=13.9 Hz, 1H), 2.12-1.71 (m, 3H), 1.46 (dd, J=23.2, 6.3 Hz, 3H).

P2: LC-MS (Method 1): Retention time=2.38 min. MS (ESI) m/z 621.2 [M+H]⁺.

¹H NMR (500 MHZ, CH₃OH-d₄) δ 7.91 (m, 2H), 7.31-6.98 (m, 5H), 6.98-6.75 (m, 3H), 5.50 (m, 2H), 2.86 (d, J=17.0 Hz, 1H), 2.61 (td, J=10.9, 5.0 Hz, 1H), 2.27 (d, J= 11.5 Hz, 1H), 2.16-1.68 (m, 3H), 1.46 (dd, J=27.5, 6.3 Hz, 3H).

### Examples 34 and 35

### 2-Methyl-4-[[1-[3-[(1R)-1-(2,2,6-trifluoro-1,3-benzodioxol-5-yl)ethoxy]phenyl]-3-(trifluoromethyl)-4,5,6,7-tetrahydroindazol-7-yl]oxy]benzoic acid

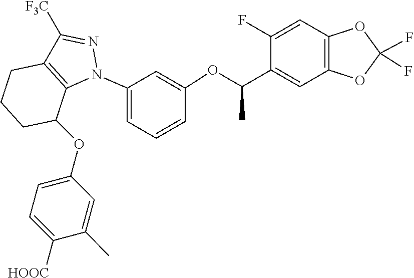 [SMILES: Cc1cc(OC2CCCc3c(C(F)(F)F)nn(-c4cccc(O[C@H](C)c5cc6c(cc5F)OC(F)(F)O6)c4)c32)ccc1C(=O)O]

### 2-methyl-4-[[1-[3-[(1S)-1-(2,2,6-trifluoro-1,3-benzodioxol-5-yl)ethoxy]phenyl]-3-(trifluoromethyl)-4,5,6,7-tetrahydroindazol-7-yl]oxy]benzoic acid

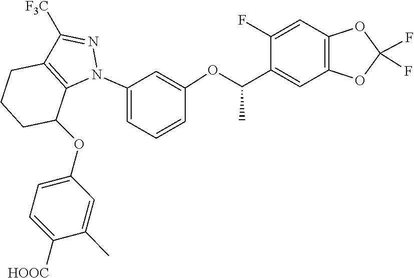 [SMILES: Cc1cc(OC2CCCc3c(C(F)(F)F)nn(-c4cccc(O[C@@H](C)c5cc6c(cc5F)OC(F)(F)O6)c4)c32)ccc1C(=O)O]

The title mixture of diastereomers were synthesized essentially in the same manner as Examples 21 and 22.

P1: LC-MS (Method 4): Retention time=1.99 min. MS (ESI) m/z 635.1 [M+H]⁺.

¹H NMR (400 MHZ, CH₃OH-d₄) δ:7.61-7.45 (m, 1H), 7.30-7.04 (m, 5H), 6.92-6.83 (m, 1H), 6.73-6.57 (m, 2H), 5.56 (m, 1H), 5.51-5.35 (m, 1H), 2.85 (m, 1H), 2.60 (m, 1H), 2.45 (m, 3H), 2.34-2.17 (m, 1H), 2.10-1.66 (m, 3H), 1.51 (m, 3H).

P2: LC-MS (Method 4): Retention time=2.02 min. MS (ESI) m/z 635.1 [M+H]⁺.

¹H NMR (400 MHZ, CH₃OH-d₄) δ:7.60-7.46 (m, 1H), 7.28-7.06 (m, 5H), 6.90 (m, 1H), 6.67 (m, 2H), 5.63-5.52 (m, 1H), 5.45 (m, 1H), 2.85 (m, 1H), 2.59 (s, 1H), 2.45 (dm, 2H), 2.23 (s, 1H), 1.84 (m, 3H), 1.51 (m, 3H).

### Example 36

### 4-(((S)-1-(3-((S)-1-(2,2-difluorobenzo[d][1,3]dioxol-5-yl)ethoxy)-4-fluorophenyl)-3-(trifluoromethyl)-4,5,6,7-tetrahydro-1H-indazol-7-yl)oxy)benzoic acid

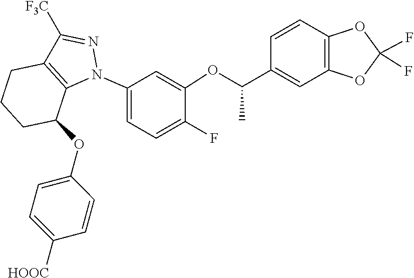 [SMILES: C[C@H](Oc1cc(-n2nc(C(F)(F)F)c3c2[C@@H](Oc2ccc(C(=O)O)cc2)CCC3)ccc1F)c1ccc2c(c1)OC(F)(F)O2]

### Step 1. Synthesis of(S)-1-(3-(1-(2,2-difluorobenzo[d][1,3]dioxol-5-yl)ethoxy)-4-fluorophenyl)-3-(trifluoromethyl)-1,4,5,6-tetrahydro-7H-indazol-7-one

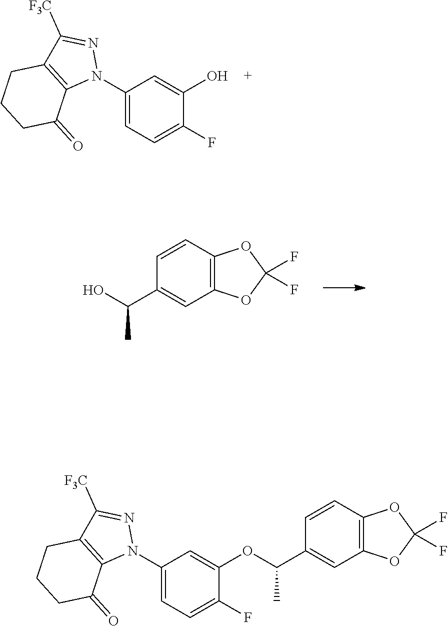

To a solution of 1-(4-fluoro-3-hydroxy-phenyl)-3-(trifluoromethyl)-5,6-dihydro-4H-indazol-7-one (Intermediate D-3) (3000 mg, 9.55 mmol) and (1R)-1-(2,2-difluoro-1,3-benzodioxol-5-yl)ethanol (Intermediate C-1R) (2123 mg, 10.50 mmol) in THF (40 mL) were added triphenylphosphine (7512 mg, 28.60 mmol) and diethyl azodicarboxylate (2.3 mL, 14.30 mmol) at 0° C. The reaction was stirred at room temperature for 2 h. The reaction mixture was concentrated in vacuum. The residue was purified by flash silica gel column chromatography (ethyl acetate in petroleum ether from 0% to 15%) to afford the title compound (4500 mg, 92.7% yield) as a brown oil.

LCMS (Method 2): Retention time=2.43 min. MS (ESI) m/z 499.0 [M+H]⁺.

### Step 2. Synthesis of (R)-1-(3-((S)-1-(2,2-difluorobenzo[d][1,3]dioxol-5-yl)ethoxy)-4-fluorophenyl)-3-(trifluoromethyl)-4,5,6,7-tetrahydro-1H-indazol-7-ol

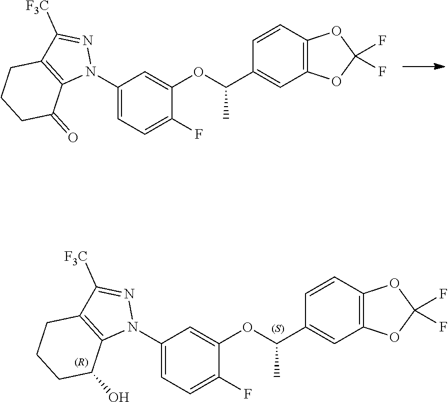

To a solution of 1-[3-[[(1S)-1-(2,2-difluoro-1,3-benzodioxol-5-yl)ethoxy]-4-fluoro-phenyl]-3-(trifluoromethyl)-5,6-dihydro-4H-indazol-7-one (4000 mg, 8.03 mmol) and ((R,R)-2-amino-1,2-diphenylethyl)[(4-tolyl)sulfonyl]amido](p-cymene)ruthenium(II)chloride (153 mg, 0.24 mmol) in IPA (50 mL) was added triethylamine (3.0 mL, 21.50 mmol) and formic acid (2.0 mL, 52.10 mmol) and stirred for 2 h. The reaction mixture was concentrated in vacuum. The residue was purified by flash silica gel column chromatography (ethyl acetate in petroleum ether from 0% to 10%) to afford the title compound (4000 mg, 97.6% yield) as a yellow oil. LCMS (Method 2): Retention time=2.37 min. MS (ESI) m/z 501.0 [M+H]⁺.

### Step 3. Synthesis of methyl 4-(((S)-1-(3-((S)-1-(2,2-difluorobenzo[d][1,3]dioxol-5-yl)ethoxy)-4-fluorophenyl)-3-(trifluoromethyl)-4,5,6,7-tetrahydro-1H-indazol-7-yl)oxy)benzoate

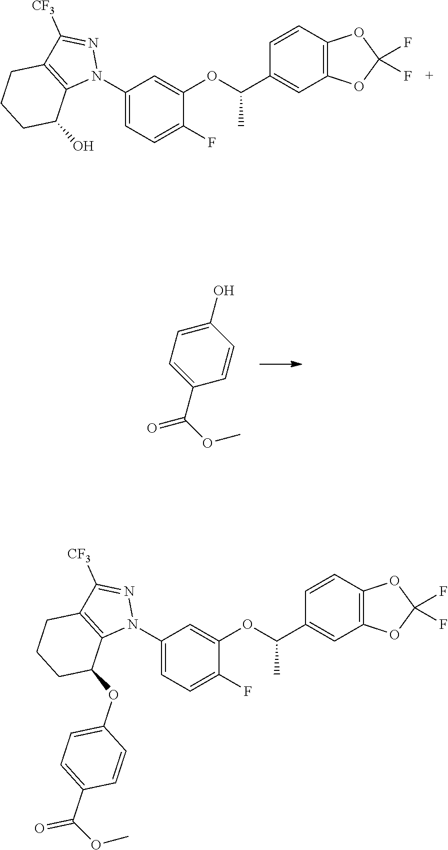

To a solution of (7R)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-ol (3000 mg, 6.00 mmol) and methyl 4-hydroxybenzoate (1003 mg, 6.59 mmol) in THF (35 mL) was added triphenylphosphine (4718 mg, 18.00 mmol) and diethyl azodicarboxylate (1.4 mL, 8.99 mmol) at 0° C. The reaction was stirred at room temperature for 2 h. The reaction mixture was concentrated in vacuum. The residue was purified by flash silica gel column chromatography (ethyl acetate in petroleum ether from 0% to 8%) to afford the title compound (3000 mg, 77.3% yield) as a light-yellow oil.

LCMS (Method 2): Retention time=2.69 min. MS (ESI) m/z 635.0 [M+H]⁺.

### Step 4. Synthesis 4-(((S)-1-(3-((S)-1-(2,2-difluorobenzo[d][1,3]dioxol-5-yl)ethoxy)-4-fluorophenyl)-3-(trifluoromethyl)-4,5,6,7-tetrahydro-1H-indazol-7-yl)oxy)benzoic acid

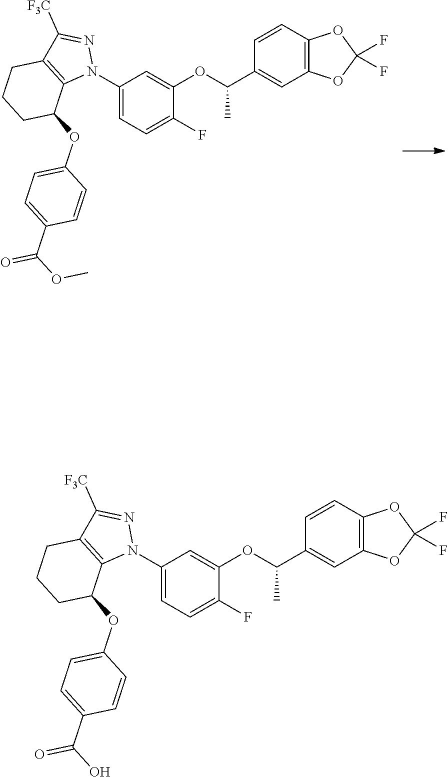 [SMILES: COC(=O)c1ccc(O[C@H]2CCCc3c(C(F)(F)F)nn(-c4ccc(F)c(O[C@@H](C)c5ccc6c(c5)OC(F)(F)O6)c4)c32)cc1 ; C[C@H](Oc1cc(-n2nc(C(F)(F)F)c3c2[C@@H](Oc2ccc(C(=O)O)cc2)CCC3)ccc1F)c1ccc2c(c1)OC(F)(F)O2]

To a solution of methyl 4-[(7S)-1-[3-[[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]benzoate (3000 mg, 4.73 mmol) in methanol (20 mL)/THF (20 mL)/water (10 mL) was added lithium hydroxide monohydrate (992 mg, 23.60 mmol). The reaction was stirred at room temperature for 2 h. The reaction mixture was concentrated in vacuum. The residue was purified by prep-HPLC (NH₄HCO₃ condition) to afford a crude product. The crude product was purified by SFC to give the title compound (2010 mg, 68.5% yield) as a white solid.

LCMS (Method 2): Retention time=1.81 min. MS (ESI) m/z 621.1 [M+H]⁺.

¹H NMR (400 MHZ, MeOH-d4) δ 8.00-7.89 (m, 2H), 7.18-7.07 (m, 4H), 7.06-6.98 (m, 2H), 6.91 (d, J=8.9 Hz, 2H), 5.46 (d, J=2.9 Hz, 1H), 5.04 (q, J=6.4 Hz, 1H), 2.84 (d, J=12.7 Hz, 1H), 2.58 (ddd, J=16.8, 10.9, 5.8 Hz, 1H), 2.22 (d, J=14.2 Hz, 1H), 2.06-1.73 (m, 3H), 1.39 (d, J=6.3 Hz, 3H).

The examples 36a to 36m were synthesized using the protocols essentially the same as Example 36.

### Example 36a

### 4-[[(7S)-1-[2-[(1S)-1-(2,2,6-trifluoro-1,3-benzodioxol-5-yl)ethoxy]-4-pyridyl]-3-(trifluoromethyl)-4,5,6,7-tetrahydroindazol-7-yl]oxy]benzoic acid

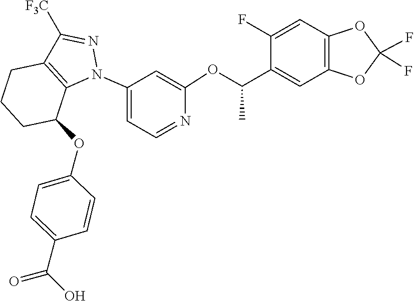 [SMILES: C[C@H](Oc1cc(-n2nc(C(F)(F)F)c3c2[C@@H](Oc2ccc(C(=O)O)cc2)CCC3)ccn1)c1cc2c(cc1F)OC(F)(F)O2]

LCMS (Method 1): Retention time=1.87 min. MS (ESI) m/z 620.0 [M−H]⁻.

Chiral Method: Column Name: OZ 4.6*100 mm 5 μm Solvent: MeOH [0.2% NH₃ (7M in MeOH)]; Find at 1.086 min; ee: 98.84%

¹H NMR (400 MHz, MeOH-d4) δ 8.04 (dd, J=15.7, 7.2 Hz, 3H), 7.17 (dd, J=5.6, 1.8 Hz, 1H), 7.12-6.99 (m, 4H), 6.89 (d, J=5.7 Hz, 1H), 6.27 (q, J=6.5 Hz, 1H), 5.78 (d, J=3.2 Hz, 1H), 2.87 (d, J=16.8 Hz, 1H), 2.72-2.54 (m, 1H), 2.35-2.24 (m, 1H), 2.07-1.87 (m, 3H), 1.44 (d, J=6.5 Hz, 3H).

### Example 36b

### 4-[[(7S)-1-[4-[(1S)-1-(2,2-difluoro-1,3-benzodioxol-5-yl)ethoxy]-2-pyridyl]-3-(trifluoromethyl)-4,5,6,7-tetrahydroindazol-7-yl]oxy]benzoic acid

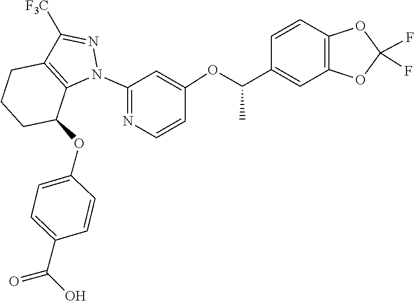 [SMILES: C[C@H](Oc1ccnc(-n2nc(C(F)(F)F)c3c2[C@@H](Oc2ccc(C(=O)O)cc2)CCC3)c1)c1ccc2c(c1)OC(F)(F)O2]

LCMS (Method 2): Retention time=1.87 min. MS (ESI) m/z 604.0 [M+H]⁺.

¹H NMR (400 MHZ, MeOH-d4) δ 7.88 (d, J=8.8 Hz, 2H), 7.65 (d, J=5.8 Hz, 1H), 7.33 (d, J=2.2 Hz, 1H), 7.25 (d, J=1.3 Hz, 1H), 7.17 (dt, J=18.2, 4.9 Hz, 2H), 6.88 (d, J=8.9 Hz, 2H), 6.71 (dd, J=5.8, 2.3 Hz, 1H), 6.42 (s, 1H), 5.57 (q, J=6.3 Hz, 1H), 2.82 (d, J=15.7 Hz, 1H), 2.58 (dd, J=14.9, 7.7 Hz, 1H), 2.28-2.13 (m, 1H), 2.03-1.81 (m, 3H), 1.60 (d, J=6.4 Hz, 3H).

### Example 36c

### 4-[[(7S)-1-[6-[(1S)-1-(2,2-difluoro-1,3-benzodioxol-5-yl)ethoxy]-2-pyridyl]-3-(trifluoromethyl)-4,5,6,7-tetrahydroindazol-7-yl]oxy]benzoic acid

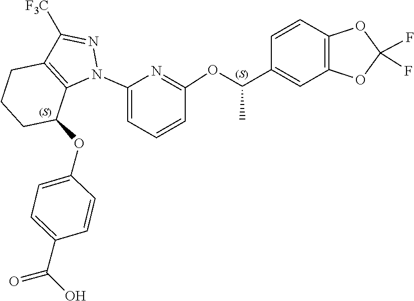

LCMS (Method 2): Retention time=1.83 min. MS (ESI) m/z 602.0 [M−H]⁺.

¹H NMR (400 MHZ, MeOH-d4) δ 7.76 (t, J=8.0 Hz, 1H), 7.70 (d, J=8.7 Hz, 2H), 7.25 (d, J=7.7 Hz, 1H), 7.16-7.00 (m, 3H), 6.73 (d, J=8.2 Hz, 1H), 6.67 (d, J=8.8 Hz, 2H), 6.07 (s, 1H), 5.77 (q, J=6.3 Hz, 1H), 2.83 (d, J=16.4 Hz, 1H), 2.71-2.55 (m, 1H), 2.24 (d, J=13.9 Hz, 1H), 2.01 (m, 3H), 1.41 (d, J=6.4 Hz, 3H).

### Example 36d

### 4-[[(7S)-1-[6-fluoro-5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]benzoic acid

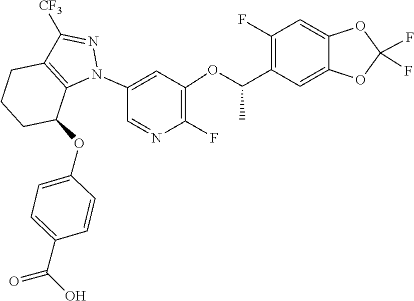 [SMILES: C[C@H](Oc1cc(-n2nc(C(F)(F)F)c3c2[C@@H](Oc2ccc(C(=O)O)cc2)CCC3)cnc1F)c1cc2c(cc1F)OC(F)(F)O2]

(31 mg, 63.16% yield) as a white solid.

LCMS (Method 2): Retention time=1.80 min. MS (ESI) m/z 640.0 [M+H]⁺.

¹H NMR (400 MHZ, MeOH-d4) δ 7.93 (d, J=8.8 Hz, 2H), 7.89 (s, 1H), 7.59 (dd, J=8.5, 2.1 Hz, 1H), 7.25 (d, J=5.7 Hz, 1H), 7.11 (d, J=9.0 Hz, 1H), 6.94 (d, J=8.8 Hz, 2H), 5.62 (s, 1H), 5.44 (q, J=6.2 Hz, 1H), 2.85 (d, J=16.5 Hz, 1H), 2.66-2.56 (m, 1H), 2.23 (d, J=11.7 Hz, 1H), 2.02-1.84 (m, 3H), 1.46 (t, J=6.9 Hz, 3H).

### Example 36e

### 4-[[(7S)-1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]benzoic acid

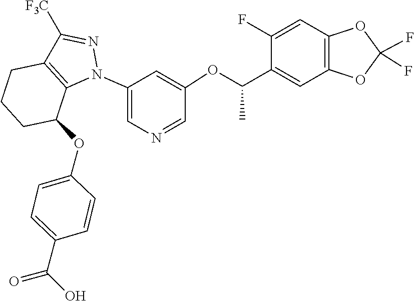 [SMILES: C[C@H](Oc1cncc(-n2nc(C(F)(F)F)c3c2[C@@H](Oc2ccc(C(=O)O)cc2)CCC3)c1)c1cc2c(cc1F)OC(F)(F)O2]

LCMS (Method2): LC retention time 1.76, MS (ESI): m/z 622 [M+H]+.

¹H NMR (400 MHZ, MeOH-d4) δ 8.33 (d, J=1.8 Hz, 1H), 8.17 (d, J=2.5 Hz, 1H), 7.96 (d, J=8.8 Hz, 2H), 7.55 (t, J=2.3 Hz, 1H), 7.12 (dd, J=10.0, 7.4 Hz, 2H), 6.98 (d, J=8.8 Hz, 2H), 5.66 (d, J=3.2 Hz, 1H), 5.55 (q, J=6.3 Hz, 1H), 2.87 (d, J=16.2 Hz, 1H), 2.71-2.49 (m, 1H), 2.24 (dd, J=14.8, 11.4 Hz, 1H), 2.11-1.82 (m, 3H), 1.47 (t, J=11.0 Hz, 3H).

### Example 36f

### 4-[[(7S)-1-[6-[(1S)-1-(2,2-difluoro-1,3-benzodioxol-5-yl)ethoxy]-5-fluoro-2-pyridyl]-3-(trifluoromethyl)-4,5,6,7-tetrahydroindazol-7-yl]oxy]benzoic acid

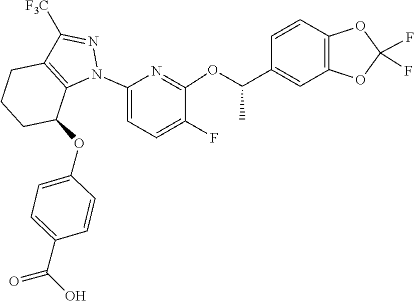 [SMILES: C[C@H](Oc1nc(-n2nc(C(F)(F)F)c3c2[C@@H](Oc2ccc(C(=O)O)cc2)CCC3)ccc1F)c1ccc2c(c1)OC(F)(F)O2]

LCMS (Method 4): Retention time=1.80 min. MS (ESI) m/z 620.1 [M−H]⁻.

¹H NMR (400 MHZ, MeOH-d4) δ 7.71 (d, J=8.8 Hz, 2H), 7.64 (dd, J=9.3, 8.5 Hz, 1H), 7.26 (dd, J=8.4, 2.6 Hz, 1H), 7.14 (d, J=8.5 Hz, 2H), 7.07 (dd, J=8.3, 1.5 Hz, 1H), 6.67 (d, J=8.8 Hz, 2H), 5.96 (t, J=3.7 Hz, 1H), 5.86 (q, J=6.4 Hz, 1H), 2.90-2.80 (m, 1H), 2.71-2.59 (m, 1H), 2.31-2.20 (m, 1H), 2.12-1.86 (m, 3H), 1.46 (d, J=6.5 Hz, 3H).

### Example 36g

### 4-[[(7S)-1-[3-[(1S)-1-(2,2,6-trifluoro-1,3-benzodioxol-5-yl)ethoxy]phenyl]-3-(trifluoromethyl)-4,5,6,7-tetrahydroindazol-7-yl]oxy]benzoic acid

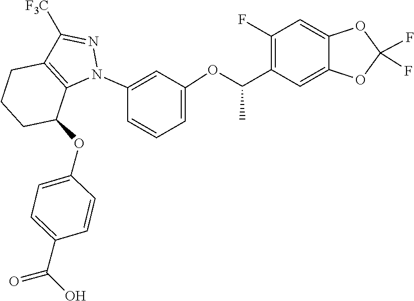 [SMILES: C[C@H](Oc1cccc(-n2nc(C(F)(F)F)c3c2[C@@H](Oc2ccc(C(=O)O)cc2)CCC3)c1)c1cc2c(cc1F)OC(F)(F)O2]

LCMS (Method 2): Retention time=1.78 min. MS (ESI) m/z 621.0 [M+H]⁺.

¹H NMR (400 MHZ, MeOH-d4) δ 7.96 (d, J=8.8 Hz, 2H), 7.23 (t, J=8.1 Hz, 1H), 7.08 (dd, J=15.3, 8.4 Hz, 3H), 6.98 (d, J=8.9 Hz, 2H), 6.93 (d, J=5.7 Hz, 1H), 6.83 (dd, J=8.3, 1.9 Hz, 1H), 5.53 (s, 1H), 5.47 (q, J=6.3 Hz, 1H), 2.88 (d, J=15.3 Hz, 1H), 2.68-2.56 (m, 1H), 2.27 (d, J=13.7 Hz, 1H), 2.09-1.81 (m, 3H), 1.43 (d, J=6.3 Hz, 3H).

### Example 36h

### 4-(((S)-1-(5-((S)-1-(2,2-difluorobenzo[d][1,3]dioxol-5-yl)ethoxy)pyridazin-3-yl)-3-(trifluoromethyl)-4,5,6,7-tetrahydro-1H-indazol-7-yl)oxy)benzoic acid

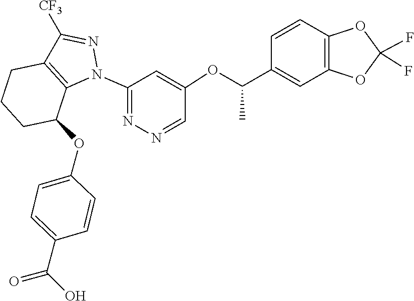 [SMILES: C[C@H](Oc1cnnc(-n2nc(C(F)(F)F)c3c2[C@@H](Oc2ccc(C(=O)O)cc2)CCC3)c1)c1ccc2c(c1)OC(F)(F)O2]

Example 36h was synthesized using the similar procedures as Example 36 by condensing Intermediate D-9 with Intermediate C-1R. After chiral reduction and coupling, followed by ester hydrolysis to obtain the title compound in 44.34% yield as a white solid.

LCMS (Method 2): Retention time=1.79 min. MS (ESI) m/z 603.0 [M−H]⁻.

¹H NMR (400 MHZ, MeOH-d4) δ 8.76 (d, J=2.6 Hz, 1H), 7.91 (d, J=8.8 Hz, 2H), 7.55 (d, J=2.6 Hz, 1H), 7.36 (d, J=1.4 Hz, 1H), 7.28 (dd, J=8.3, 1.6 Hz, 1H), 7.20 (d, J=8.3 Hz, 1H), 6.96 (d, J=8.8 Hz, 2H), 6.56 (s, 1H), 5.75 (q, J=6.3 Hz, 1H), 2.84 (d, J=15.9 Hz, 1H), 2.70-2.53 (m, 1H), 2.32-2.17 (m, 1H), 2.07-1.82 (m, 3H), 1.68 (d, J=6.4 Hz, 3H).

### Example 36i

### 4-[[(7S)-1-[4-[(1S)-1-(2,2-difluoro-1,3-benzodioxol-5-yl)ethoxy]-5-fluoro-2-pyridyl]-3-(trifluoromethyl)-4,5,6,7-tetrahydroindazol-7-yl]oxy]benzoic acid

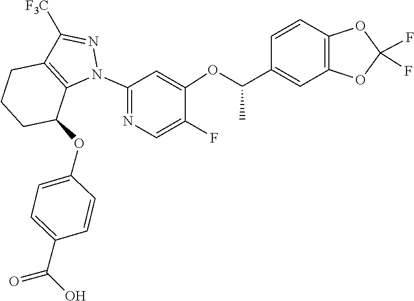 [SMILES: C[C@H](Oc1cc(-n2nc(C(F)(F)F)c3c2[C@@H](Oc2ccc(C(=O)O)cc2)CCC3)ncc1F)c1ccc2c(c1)OC(F)(F)O2]

Example 36i was synthesized using similar procedures as Example 36 by condensing Intermediate D-10 with Intermediate C-1R. After chiral reduction and coupling, followed by ester hydrolysis to obtain the title compound in 37.98% yield as a white solid.

LCMS (Method 2): Retention time=1.86 min. MS (ESI) m/z 620.0 [M−H]⁻.

¹H NMR (400 MHZ, MeOH-d4) δ 7.95 (d, J=8.8 Hz, 2H), 7.64 (d, J=2.5 Hz, 1H), 7.49 (d, J=6.1 Hz, 1H), 7.32 (d, J=1.5 Hz, 1H), 7.26 (dd, J=8.3, 1.6 Hz, 1H), 7.18 (d, J=8.3 Hz, 1H), 6.94 (d, J=8.8 Hz, 2H), 6.42 (s, 1H), 5.70 (q, J=6.3 Hz, 1H), 2.82 (d, J=16.7 Hz, 1H), 2.63-2.51 (m, 1H), 2.28-2.17 (m, 1H), 2.03-1.85 (m, 3H), 1.67 (d, J=6.4 Hz, 3H).

### Example 36j

### 4-[[(7S)-1-[4-fluoro-3-[(1S)-1-(2,2,6-trifluoro-1,3-benzodioxol-5-yl)ethoxy]phenyl]-3-(trifluoromethyl)-4,5,6,7-tetrahydroindazol-7-yl]oxy]benzoic acid

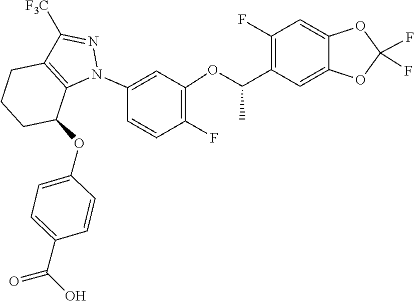 [SMILES: C[C@H](Oc1cc(-n2nc(C(F)(F)F)c3c2[C@@H](Oc2ccc(C(=O)O)cc2)CCC3)ccc1F)c1cc2c(cc1F)OC(F)(F)O2]

LCMS (Method 2): Retention time=1.83 min. MS (ESI) m/z 639.0 [M+H]⁺.

¹H NMR (400 MHZ, MeOH) δ 7.92 (d, J=8.8 Hz, 2H), 7.23-7.14 (m, 2H), 7.14-7.00 (m, 3H), 6.92 (d, J=8.8 Hz, 2H), 5.50 (s, 1H), 5.35 (q, J=6.2 Hz, 1H), 2.84 (d, J=14.1 Hz, 1H), 2.63-2.53 (m, 1H), 2.22 (d, J=13.9 Hz, 1H), 1.98-1.82 (m, 3H), 1.40 (d, J=6.3 Hz, 3H).

### Example 36k

### 4-[[(7S)-1-[2-[(1S)-1-(2,2-difluoro-1,3-benzodioxol-5-yl)propoxy]-4-pyridyl]-3-(trifluoromethyl)-4,5,6,7-tetrahydroindazol-7-yl]oxy]benzoic acid

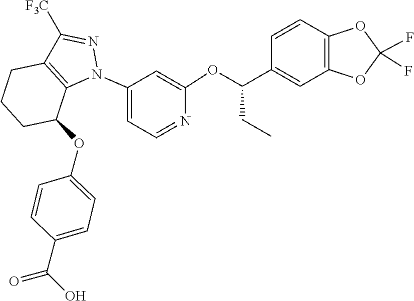 [SMILES: CC[C@H](Oc1cc(-n2nc(C(F)(F)F)c3c2[C@@H](Oc2ccc(C(=O)O)cc2)CCC3)ccn1)c1ccc2c(c1)OC(F)(F)O2]

LCMS (Method 2): Retention time=1.90 min. MS (ESI) m/z 616.0 [M−H]⁺.

¹H NMR (400 MHZ, MeOH-d4) δ 8.09-7.98 (m, 3H), 7.14 (dd, J=5.6, 1.8 Hz, 1H), 7.07 (dd, J=8.6, 3.7 Hz, 3H), 7.01-6.96 (m, 2H), 6.92 (dd, J=8.3, 1.5 Hz, 1H), 5.81 (t, J=6.5 Hz, 1H), 5.74 (s, 1H), 2.87 (d, J=15.9 Hz, 1H), 2.70-2.53 (m, 1H), 2.29 (d, J=11.8 Hz, 1H), 2.08-1.65 (m, 5H), 0.79 (t, J=7.4 Hz, 3H).

### Example 36l

### 4-[[(7S)-1-[6-[(1S)-1-(2,2,6-trifluoro-1,3-benzodioxol-5-yl)ethoxy]-2-pyridyl]-3-(trifluoromethyl)-4,5,6,7-tetrahydroindazol-7-yl]oxy]benzoic acid

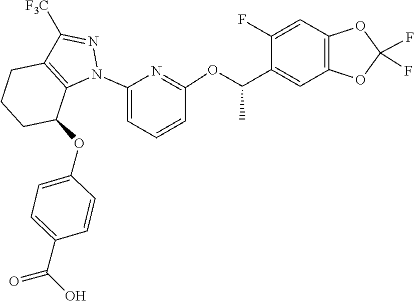 [SMILES: C[C@H](Oc1cccc(-n2nc(C(F)(F)F)c3c2[C@@H](Oc2ccc(C(=O)O)cc2)CCC3)n1)c1cc2c(cc1F)OC(F)(F)O2]

LCMS (Method 4): Retention time=1.81 min. MS (ESI) m/z 620.1 [M−H]⁺.

### Example 36m

### 4-[[(7S)-1-[5-fluoro-6-[(1S)-1-(2,2,6-trifluoro-1,3-benzodioxol-5-yl)ethoxy]-2-pyridyl]-3-(trifluoromethyl)-4,5,6,7-tetrahydroindazol-7-yl]oxy]benzoic acid

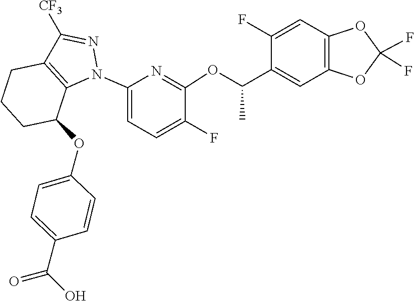 [SMILES: C[C@H](Oc1nc(-n2nc(C(F)(F)F)c3c2[C@@H](Oc2ccc(C(=O)O)cc2)CCC3)ccc1F)c1cc2c(cc1F)OC(F)(F)O2]

LCMS (Method 4): Retention time=1.81 min. MS (ESI) m/z 638.2 [M−H]⁺.

¹H NMR (400 MHZ, MeOH-d4) δ 7.72-7.61 (m, 3H), 7.32-7.23 (m, 2H), 7.16 (d, J=9.1 Hz, 1H), 6.61 (d, J=8.8 Hz, 2H), 6.10 (q, J=6.4 Hz, 1H), 5.95 (t, J=4.0 Hz, 1H), 2.83 (dt, J=16.0, 4.4 Hz, 1H), 2.73-2.60 (m, 1H), 2.30-2.20 (m, 1H), 2.19-2.08 (m, 1H), 2.03-1.87 (m, 2H), 1.49 (d, J=6.4 Hz, 3H).

### Examples 37 and 38

### Trans-4-((1-(3-((R)-1-(2,2-difluorobenzo[d][1,3]dioxol-5-yl)ethoxy)phenyl)-3-(trifluoromethyl)-4,5,6,7-tetrahydro-1H-indazol-7-yl)oxy)cyclohexane-1-carboxylic acid

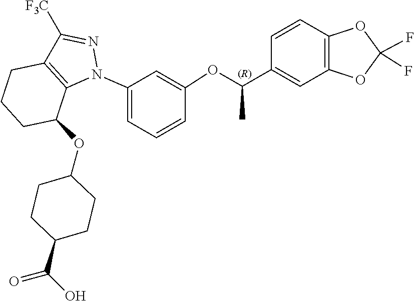

### Trans-4-((1-(3-((S)-1-(2,2-difluorobenzo[d][1,3]dioxol-5-yl)ethoxy)phenyl)-3-(trifluoromethyl)-4,5,6,7-tetrahydro-1H-indazol-7-yl)oxy)cyclohexane-1-carboxylic acid

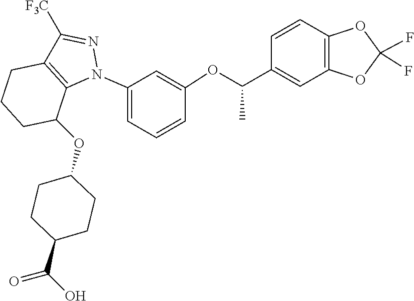 [SMILES: C[C@H](Oc1cccc(-n2nc(C(F)(F)F)c3c2C(O[C@H]2CC[C@H](C(=O)O)CC2)CCC3)c1)c1ccc2c(c1)OC(F)(F)O2]

### Step 1. Synthesis of (R)-1-(3-(1-(2,2-difluorobenzo[d][1,3]dioxol-5-yl)ethoxy)phenyl)-3-(trifluoromethyl)-1,4,5,6-tetrahydro-7H-indazol-7-one and(S)-1-(3-(1-(2,2-difluorobenzo[d][1,3]dioxol-5-yl)ethoxy)phenyl)-3-(trifluoromethyl)-1,4,5,6-tetrahydro-7H-indazol-7-one

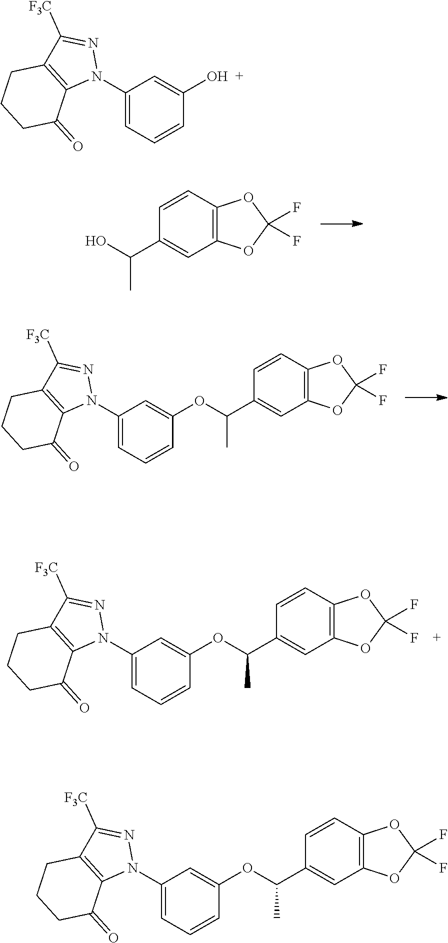

To a solution of 1-(2,2-difluoro-1,3-benzodioxol-5-yl)ethanol (Intermediate C-1) (1.84 g, 9.11 mmol), 1-(3-hydroxyphenyl)-3-(trifluoromethyl)-5,6-dihydro-4H-indazol-7-one (Intermediate D-2) (1.80 g, 6.08 mmol), and PPh₃ (2.39 g, 9.11 mmol) in THF (20.0 mL) was added DEAD (1.9 mL, 12.2 mmol) at 0° C. The reaction was stirred for 2 h. The solution was then diluted with water (30.0 mL) and extracted with EA (3×50 mL). The organic layer was washed with brine (30 mL), dried over Na₂SO₄, and concentrated. The residue was purified by chiral prep-HPLC (water with 0.01% TFA (A)/ACN (B), flow rate: 80 mL/min.) to yield the title compounds with the first fraction designated as P1 (480 mg, 37% yield; a light-yellow oil) and the second fraction designated as P2 (450 mg, 37% yield; a light-yellow oil).

LC-MS (Method 1): Retention time=2.38 min. MS (ESI) m/z 481.2 [M+H]⁺.

### Step 2a. Synthesis of 1-(3-((R)-1-(2,2-difluorobenzo[d][1,3]dioxol-5-yl)ethoxy)phenyl)-3-(trifluoromethyl)-4,5,6,7-tetrahydro-1H-indazol-7-ol

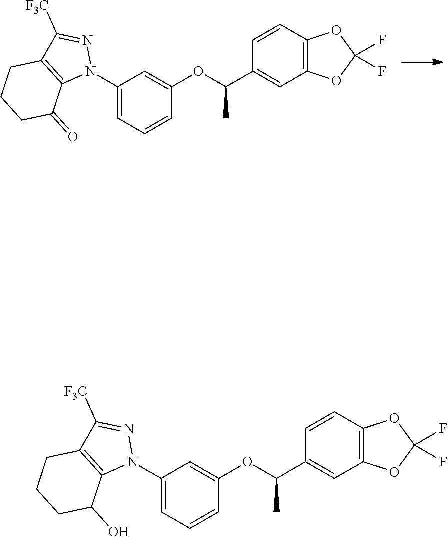 [SMILES: C[C@@H](Oc1cccc(-n2nc(C(F)(F)F)c3c2C(=O)CCC3)c1)c1ccc2c(c1)OC(F)(F)O2 ; C[C@@H](Oc1cccc(-n2nc(C(F)(F)F)c3c2C(O)CCC3)c1)c1ccc2c(c1)OC(F)(F)O2]

To a solution of (R)-1-(3-(1-(2,2-difluorobenzo[d][1,3]dioxol-5-yl)ethoxy)phenyl)-3-(trifluoromethyl)-1,4,5,6-tetrahydro-7H-indazol-7-one (the intermediate P1 from Step 1) (370 mg, 0.770 mmol) in methanol (4.0 mL) was added NaBH₄ (44 mg, 1.16 mmol) at 0° C. The solution was then stirred at rt for 1 h under Ar. The reaction was diluted with water (10 mL) and extracted with EA (3×10 mL). The organic layer was washed with brine (10 mL), dried over Na₂SO₄, and concentrated. The residue was purified by flash chromatography (Biotage, 40 g silica gel column @60 mL/min, eluted with 0-30% ethyl acetate in petroleum ether for 20 min) to yield the title mixture of diastereomers (350 mg, 94% yield) as light-yellow oil.

LC-MS (Method 1): Retention time=2.07 min. MS (ESI) m/z 483.1 [M+H]⁺.

### Step 2b. Synthesis of 1-(3-((S)-1-(2,2-difluorobenzo[d][1,3]dioxol-5-yl)ethoxy)phenyl)-3-(trifluoromethyl)-4,5,6,7-tetrahydro-1H-indazol-7-ol

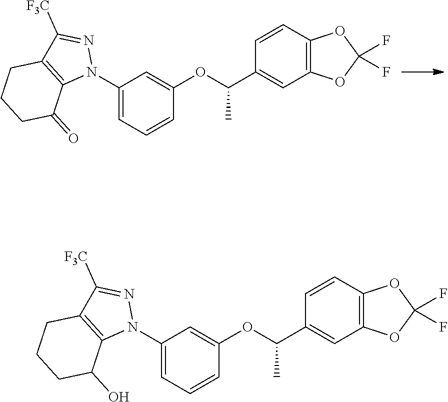 [SMILES: C[C@H](Oc1cccc(-n2nc(C(F)(F)F)c3c2C(=O)CCC3)c1)c1ccc2c(c1)OC(F)(F)O2 ; C[C@H](Oc1cccc(-n2nc(C(F)(F)F)c3c2C(O)CCC3)c1)c1ccc2c(c1)OC(F)(F)O2]

The title compound was obtained (380 mg, 94% yield) as a light-yellow oil by the same protocol as described in Step 2a using the starting material of(S)-1-(3-(1-(2,2-difluorobenzo[d][1,3]dioxol-5-yl)ethoxy)phenyl)-3-(trifluoromethyl)-1,4,5,6-tetrahydro-7H-indazol-7-one (the intermediate P2 from Step 1).

LC-MS (Method 1): Retention time=2.09 min. MS (ESI) m/z 483.3 [M+H]⁺.

### Step 3a. Synthesis of 7-chloro-1-(3-((R)-1-(2,2-difluorobenzo[d][1,3]dioxol-5-yl)ethoxy)phenyl)-3-(trifluoromethyl)-4,5,6,7-tetrahydro-1H-indazole

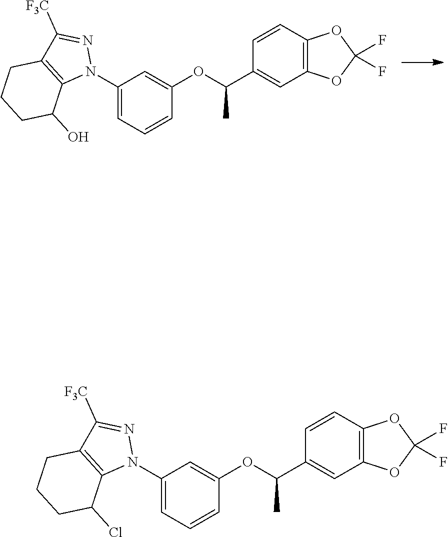 [SMILES: C[C@@H](Oc1cccc(-n2nc(C(F)(F)F)c3c2C(O)CCC3)c1)c1ccc2c(c1)OC(F)(F)O2 ; C[C@@H](Oc1cccc(-n2nc(C(F)(F)F)c3c2C(Cl)CCC3)c1)c1ccc2c(c1)OC(F)(F)O2]

To a solution of 1-(3-((R)-1-(2,2-difluorobenzo[d][1,3]dioxol-5-yl)ethoxy)phenyl)-3-(trifluoromethyl)-4,5,6,7-tetrahydro-1H-indazol-7-ol (product obtained in Step 2a) (150 mg, 0.249 mmol) and TEA (0.2 mL, 0.622 mmol) in DCM (2 mL) was added MsCl (0.1 mL, 0.373 mmol). Then the solution was stirred at rt for 2 h under Ar. The residue was concentrated under vacuum and purified by flash chromatography (Biotage, 40 g silica gel column @ 60 mL/min, eluted with 0-30% ethyl acetate in petroleum for 20 min) to give the title compound (120 mg, 77% yield) as a light-yellow oil.

LC-MS (Method 2): Retention time=2.16 min. MS (ESI) m/z 501.4 [M+H]⁺.

### Step 3b. Synthesis of 7-chloro-1-(3-((S)-1-(2,2-difluorobenzo[d][1,3]dioxol-5-yl)ethoxy)phenyl)-3-(trifluoromethyl)-4,5,6,7-tetrahydro-1H-indazole

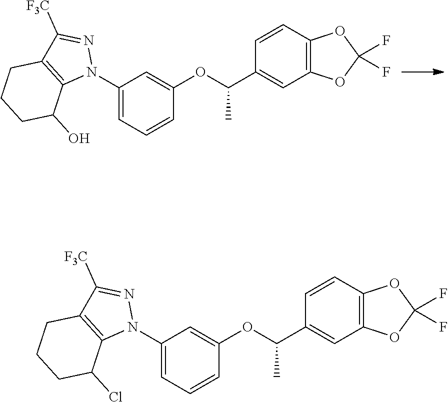 [SMILES: C[C@H](Oc1cccc(-n2nc(C(F)(F)F)c3c2C(O)CCC3)c1)c1ccc2c(c1)OC(F)(F)O2 ; C[C@H](Oc1cccc(-n2nc(C(F)(F)F)c3c2C(Cl)CCC3)c1)c1ccc2c(c1)OC(F)(F)O2]

The title compound was obtained (95 mg, 76% yield) as a light-yellow oil by the same protocol as described in Step 3a using the starting material of 1-(3-((S)-1-(2,2-difluorobenzo[d][1,3]dioxol-5-yl)ethoxy)phenyl)-3-(trifluoromethyl)-4,5,6,7-tetrahydro-1H-indazol-7-ol (product obtained in Step 2b) (120 mg, 0.249 mmol).

LC-MS (Method 2): Retention time=2.14 min. MS (ESI) m/z 501.1 [M+H]⁺.

### Step 4a. Synthesis of trans-ethyl 4-((1-(3-((R)-1-(2,2-difluorobenzo[d][1,3]dioxol-5-yl)ethoxy)phenyl)-3-(trifluoromethyl)-4,5,6,7-tetrahydro-1H-indazol-7-yl)oxy)cyclohexane-1-carboxylate

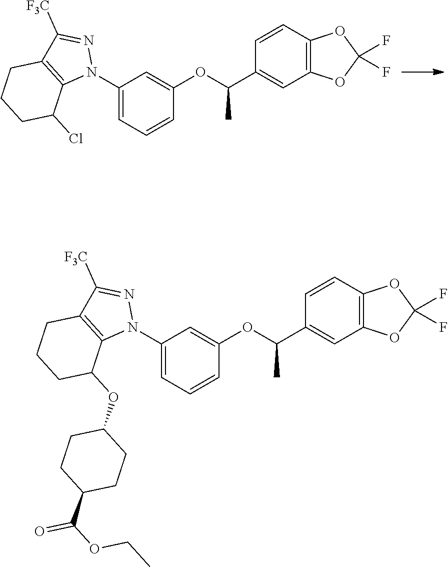 [SMILES: C[C@@H](Oc1cccc(-n2nc(C(F)(F)F)c3c2C(Cl)CCC3)c1)c1ccc2c(c1)OC(F)(F)O2 ; CCOC(=O)[C@H]1CC[C@H](OC2CCCc3c(C(F)(F)F)nn(-c4cccc(O[C@H](C)c5ccc6c(c5)OC(F)(F)O6)c4)c32)CC1]

To a solution of 7-chloro-1-(3-((R)-1-(2,2-difluorobenzo[d][1,3]dioxol-5-yl)ethoxy)phenyl)-3-(trifluoromethyl)-4,5,6,7-tetrahydro-1H-indazole (product obtained in Step 3a) (120 mg, 0.240 mmol), ethyl trans-4-hydroxycyclohexanecarboxylate (65 mg, 0.379 mmol) in DCM (1 mL) were added 2,6-di-tert-butylpyridine (91 mg, 0.474 mmol) and silver trifluoromethanesulfonate (73 mg, 0.285 mmol). Then the solution was stirred at 0° C. for 2 h under Ar. The solution was filtered. The filtrate was concentrated under vacuum. The residue was purified by HPLC (Biotage, 40 g silica gel column @ 60 mL/min, eluted with 0-100% ethyl acetate in petroleum for 20 min) to give the title compound (64 mg, 43% yield) as a light-yellow oil.

LC-MS (Method 2): Retention time=2.25 min. MS (ESI) m/z 637.3 [M+H]⁺.

### Step 4b. Synthesis of ethyl trans-4-((1-(3-((S)-1-(2,2-difluorobenzo[d][1,3]dioxol-5-yl)ethoxy)phenyl)-3-(trifluoromethyl)-4,5,6,7-tetrahydro-1H-indazol-7-yl)oxy)cyclohexane-1-carboxylate

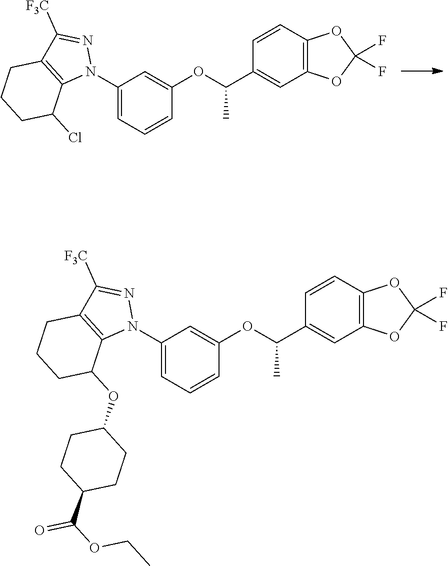 [SMILES: C[C@H](Oc1cccc(-n2nc(C(F)(F)F)c3c2C(Cl)CCC3)c1)c1ccc2c(c1)OC(F)(F)O2 ; CCOC(=O)[C@H]1CC[C@H](OC2CCCc3c(C(F)(F)F)nn(-c4cccc(O[C@@H](C)c5ccc6c(c5)OC(F)(F)O6)c4)c32)CC1]

The title compound was obtained (50 mg, 41% yield) as a light-yellow oil by the same protocol as described in Step 4a using the starting material of 7-chloro-1-(3-((S)-1-(2,2-difluorobenzo[d][1,3]dioxol-5-yl)ethoxy)phenyl)-3-(trifluoromethyl)-4,5,6,7-tetrahydro-1H-indazole (product obtained in Step 3b).

LC-MS (Method 2): Retention time=2.27 min. MS (ESI) m/z 637.5 [M+H]⁺.

### Step 5a. Synthesis of trans-4-((1-(3-((R)-1-(2,2-difluorobenzo[d][1,3]dioxol-5-yl)ethoxy)phenyl)-3-(trifluoromethyl)-4,5,6,7-tetrahydro-1H-indazol-7-yl)oxy)cyclohexane-1-carboxylic acid

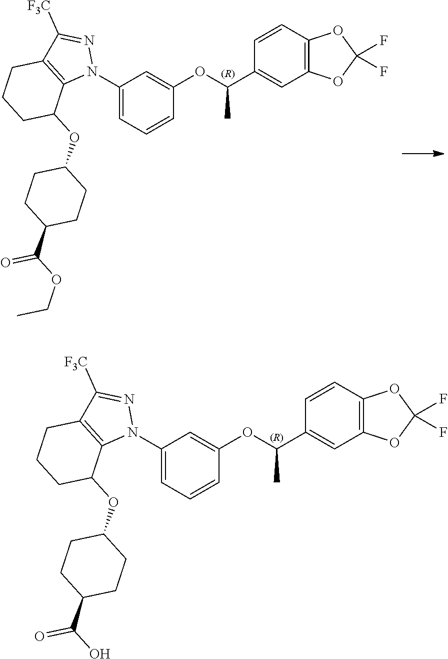

To a solution of ethyl trans-4-((1-(3-((R)-1-(2,2-difluorobenzo[d][1,3]dioxol-5-yl)ethoxy)phenyl)-3-(trifluoromethyl)-4,5,6,7-tetrahydro-1H-indazol-7-yl)oxy)cyclohexane-1-carboxylate (the product obtained in Step 4a) (64 mg, 0.0785 mmol) in methanol (1 mL), THF (1 mL), was added LiOH (2.0 mL, 0.785 mmol) at 0° C. Then the solution was stirred at rt for 1 h under N₂. To this was added aqueous HCl (2M, 2 ml) and the pH was adjusted to 7. Then the solution was extracted with EA (3×5 mL). The organic layer was washed with brine (3 mL), dried over Na₂SO₄, and concentrated under vacuum. The residue was purified by pre-HPLC to get the title compound (27.6 mg, 41% yield) as a white solid.

LC-MS (Method 2): Retention time=1.88 min. MS (ESI) m/z 609.0 [M+H]⁺.

¹H NMR (500 MHZ, CH₃OH-d₄) δ 7.42-7.36 (m, 1H), 7.34-6.99 (m, 6H), 5.55-5.49 (m, 1H), 4.73 (s, 0.5H), 4.34 (s, 0.5H), 3.15-3.08 (m, 1H), 2.75-2.68 (m, 1H), 2.54-2.47 (m, 1H), 2.16-1.96 (m, 2H), 1.94-1.53 (m, 9H), 1.46-0.96 (m, 4H), 0.74-0.49 (m, 1H).

### Step 5b. Synthesis of trans-4-((1-(3-((S)-1-(2,2-difluorobenzo[d][1,3]dioxol-5-yl)ethoxy)phenyl)-3-(trifluoromethyl)-4,5,6,7-tetrahydro-1H-indazol-7-yl)oxy)cyclohexane-1-carboxylic acid

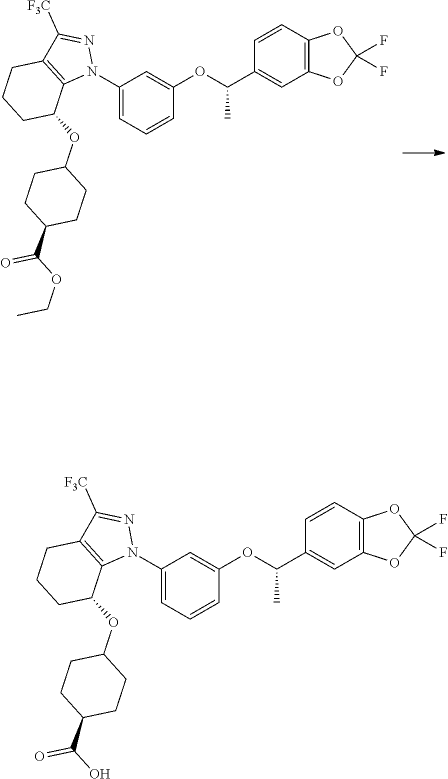

The title mixture of diastereomers was obtained (12.4 mg, 26% yield) as a white solid using the same protocol as described in Step 5a by hydrolyzing ethyl trans-4-((1-(3-((S)-1-(2,2-difluorobenzo[d][1,3]dioxol-5-yl)ethoxy)phenyl)-3-(trifluoromethyl)-4,5,6,7-tetrahydro-1H-indazol-7-yl)oxy)cyclohexane-1-carboxylate (the product obtained in Step 4b)

LC-MS (Method 2): Retention time=1.91 min. MS (ESI) m/z 609.0 [M+H]⁺.

¹H NMR (500 MHZ, CH₃OH-d₄) δ 7.42-7.36 (m, 1H), 7.35-7.02 (m, 6H), 5.56-5.46 (m, 1H), 4.74 (s, 0.5H), 4.34 (s, 0.5H), 3.15-3.10 (m, 0.5H), 2.91-2.86 (m, 0.5H), 2.78-2.70 (m, 1H), 2.56-2.48 (m, 1H), 2.10-1.97 (m, 2H), 1.95-1.55 (m, 9H), 1.34-1.24 (m, 1.5H), 1.18-0.98 (m, 2.5H), 0.77-0.61 (m, 1H).

### Examples 39 and 40

### 3-((1-(2-((R)-1-(2,2-difluorobenzo[d][1,3]dioxol-5-yl)ethoxy)pyridin-4-yl)-3-(trifluoromethyl)-4,5,6,7-tetrahydro-1H-indazol-7-yl)oxy)bicyclo[1.1.1]pentane-1-carboxylic acid

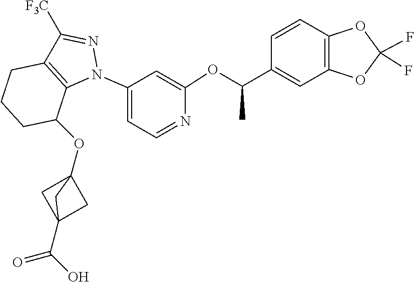 [SMILES: C[C@@H](Oc1cc(-n2nc(C(F)(F)F)c3c2C(OC24CC(C(=O)O)(C2)C4)CCC3)ccn1)c1ccc2c(c1)OC(F)(F)O2]

### 3-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylic acid

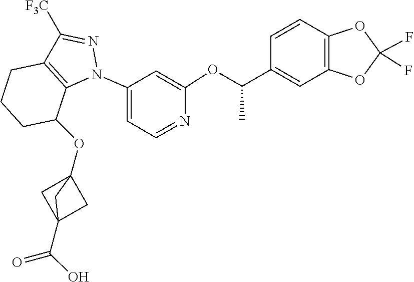 [SMILES: C[C@H](Oc1cc(-n2nc(C(F)(F)F)c3c2C(OC24CC(C(=O)O)(C2)C4)CCC3)ccn1)c1ccc2c(c1)OC(F)(F)O2]

### Step 1a. Synthesis of 7-chloro-1-(2-((R)-1-(2,2-difluorobenzo[d][1,3]dioxol-5-yl)ethoxy)pyridin-4-yl)-3-(trifluoromethyl)-4,5,6,7-tetrahydro-1H-indazole

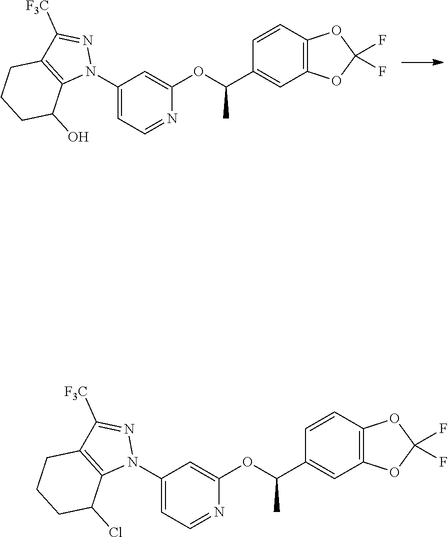 [SMILES: C[C@@H](Oc1cc(-n2nc(C(F)(F)F)c3c2C(O)CCC3)ccn1)c1ccc2c(c1)OC(F)(F)O2 ; C[C@@H](Oc1cc(-n2nc(C(F)(F)F)c3c2C(Cl)CCC3)ccn1)c1ccc2c(c1)OC(F)(F)O2]

To a solution of 1-[2-[(1R)-1-(2,2-difluoro-1,3-benzodioxol-5-yl)ethoxy]-4-pyridyl]-3-(trifluoromethyl)-4,5,6,7-tetrahydroindazol-7-ol (Intermediate from Examples 21 and 22, Step 2a) (200 mg, 0.414 mmol)) in DCM (5 mL) was added MsCl (0.039 mL, 0.496 mmol) and TEA (0.17 mL, 1.24 mmol). Then the solution was stirred at rt for 2 h under Ar. The solution was quenched with water. The solution was diluted with water (10 mL) and extracted with EA (3×50 mL). The organic layer was washed with brine (10 mL) and dried over Na₂SO₄, filtered, and concentrated under vacuum, and the crude product purified by flash chromatography (Biotage, 40 g silica gel column @ 60 mL/min, eluted with 0-30% ethyl acetate in petroleum ether for 20 min) to get the title compound (200 mg, 51% yield) as a light-yellow oil.

LC-MS (Method 1): Retention time=2.18 min. MS (ESI) m/z 318.1 (M+H)⁺.

### Step 1b. Synthesis of 7-chloro-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazole

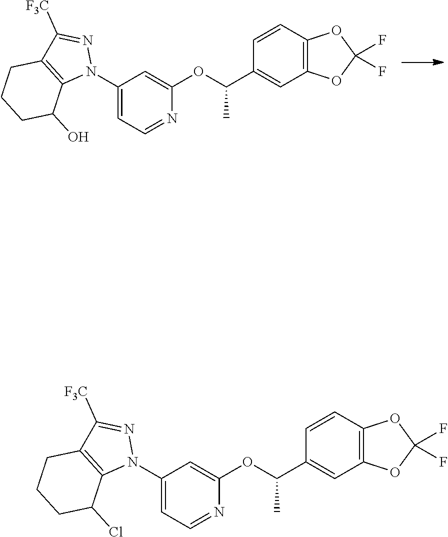 [SMILES: C[C@H](Oc1cc(-n2nc(C(F)(F)F)c3c2C(O)CCC3)ccn1)c1ccc2c(c1)OC(F)(F)O2 ; C[C@H](Oc1cc(-n2nc(C(F)(F)F)c3c2C(Cl)CCC3)ccn1)c1ccc2c(c1)OC(F)(F)O2]

The title compound was obtained (200 mg, 51% yield) as a light-yellow oil by the same protocol as described in Step 1a using 1-[2-[(1R)-1-(2,2-difluoro-1,3-benzodioxol-5-yl)ethoxy]-4-pyridyl]-3-(trifluoromethyl)-4,5,6,7-tetrahydroindazol-7-ol (intermediate from Examples 21 and 22, Step 2b) (200 mg, 0.414 mmol) as a starting material.

LC-MS (Method 1): Retention time=2.18 min. MS (ESI) m/z 318.1 (M+H)⁺.

### Step 2a. Synthesis of methyl 3-((1-(2-((R)-1-(2,2-difluorobenzo[d][1,3]dioxol-5-yl)ethoxy)pyridin-4-yl)-3-(trifluoromethyl)-4,5,6,7-tetrahydro-1H-indazol-7-yl)oxy)bicyclo[1.1.1]pentane-1-carboxylate

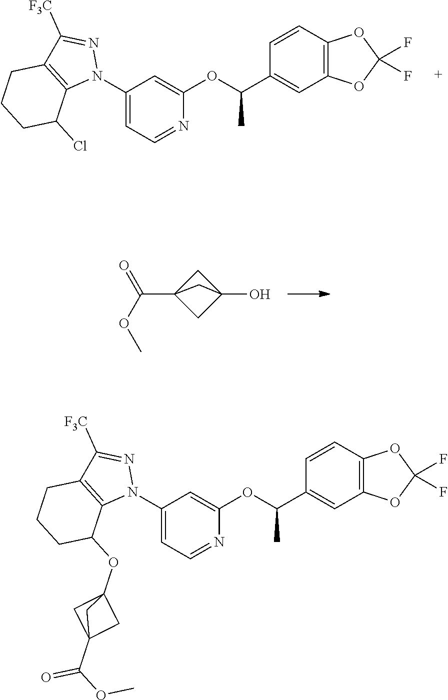

To a solution of 2,6-di-tert-butylpyridine (95 mg, 0.498 mmol)) in DCM (5 mL) was added 7-chloro-1-[2-[(1R)-1-(2,2-difluoro-1,3-benzodioxol-5-yl)ethoxy]-4-pyridyl]-3-(trifluoromethyl)-4,5,6,7-tetrahydroindazole (product of Step 1a) (100 mg, 0.199 mmol), methyl 3-hydroxybicyclo[1.1.1]pentane-1-carboxylate (31 mg, 0.219 mmol) and AgOTf (77 mg, 0.299 mmol). Then the solution was stirred at rt for 2 h under Ar. The reaction was quenched with water. The solution was diluted with water (10 mL) and extracted with EA (3×50 mL). The organic layer was washed with brine (10 mL) and dried over Na₂SO₄ and concentrated under vacuum. The residue was purified by flash chromatography (Biotage, 40 g silica gel column @ 60 mL/min, eluted with 0-30% ethyl acetate in petroleum for 20 min) to get the title compound (50 mg, 31% yield) as a white solid.

LC-MS (Method 1): Retention time=2.22 min. MS (ESI) m/z 424.1 [M-183]⁺.

### Step 2b. Synthesis of methyl 3-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylate

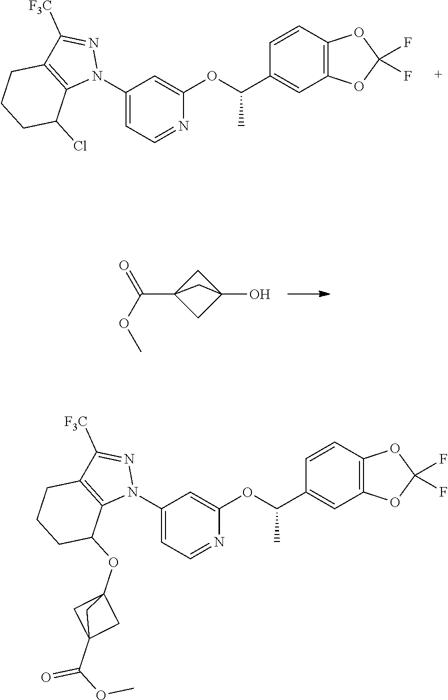

The title compound was obtained (60 mg, 33.21% yield) as a white solid by the same protocol as described in Step 2a using 7-chloro-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazole (product of Step 1b) as a starting material.

LC-MS (Method 1): Retention time=2.22 min. MS (ESI) m/z 424.1 (M-183)⁺.

### Step 3a. Synthesis of 3-((1-(2-(R-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylic acid

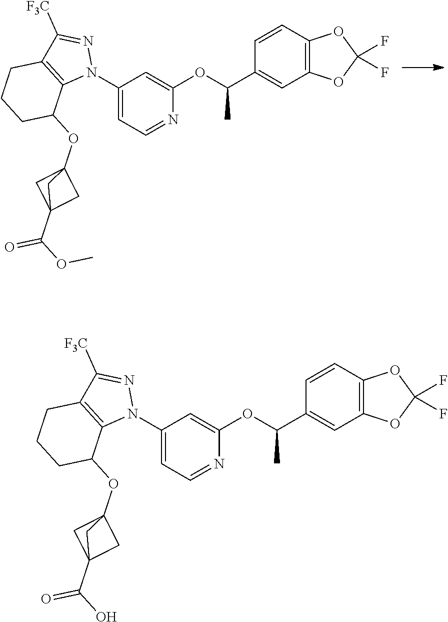 [SMILES: COC(=O)C12CC(OC3CCCc4c(C(F)(F)F)nn(-c5ccnc(O[C@H](C)c6ccc7c(c6)OC(F)(F)O7)c5)c43)(C1)C2 ; C[C@@H](Oc1cc(-n2nc(C(F)(F)F)c3c2C(OC24CC(C(=O)O)(C2)C4)CCC3)ccn1)c1ccc2c(c1)OC(F)(F)O2]

To a solution of methyl 3-[[1-[2-[(1R)-1-(2,2-difluoro-1,3-benzodioxol-5-yl)ethoxy]-4-pyridyl]-3-(trifluoromethyl)-4,5,6,7-tetrahydroindazol-7-yl]oxy]bicyclo[1.1.1]pentane-1-carboxylate (product from Step 2a) (40 mg, 0.0658 mmol) in a mixture of methanol (5 mL) and water (1 mL) was added lithium hydroxide monohydrate (11 mg, 0.263 mmol). The reaction mixture was stirred at rt for 2 h. The solution was concentrated under vacuum and purified by prep-HPLC to get the title compound (20 mg, 52% yield) as a white solid.

LC-MS (Method 1): Retention time=1.78 min. MS (ESI) m/z 592.0 (M−H)⁺.

¹H NMR (500 MHZ, CD₃OD) δ 8.22 (d, J=5.6 Hz, 1H), 7.35-7.25 (m, 2H), 7.24-7.20 (m, 1H), 7.18 (dd, J=7.0, 1.6 Hz, 1H), 7.14 (d, J=8.3 Hz, 1H), 6.26 (dq, J=9.3, 6.5 Hz, 1H), 4.88 (dt, J=10.2, 3.5 Hz, 1H), 2.76 (m, 1H), 2.55 (d, J=10.0 Hz, 1H), 2.20 (m, 7H), 2.01-1.79 (m, 2H), 1.80-1.68 (m, 1H), 1.66 (d, J=6.5 Hz, 3H).

### Step 3b. Synthesis of 3-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylic acid

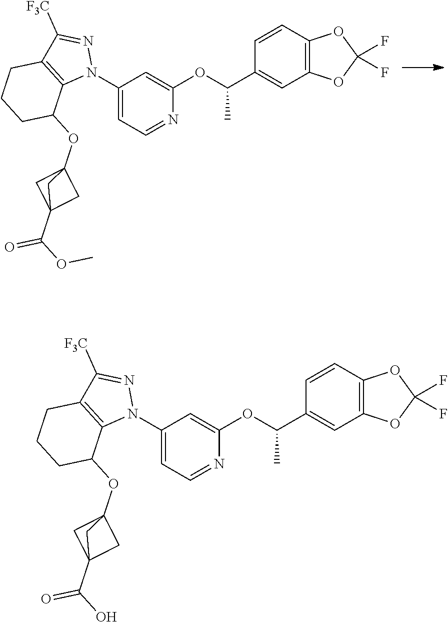 [SMILES: COC(=O)C12CC(OC3CCCc4c(C(F)(F)F)nn(-c5ccnc(O[C@@H](C)c6ccc7c(c6)OC(F)(F)O7)c5)c43)(C1)C2 ; C[C@H](Oc1cc(-n2nc(C(F)(F)F)c3c2C(OC24CC(C(=O)O)(C2)C4)CCC3)ccn1)c1ccc2c(c1)OC(F)(F)O2]

The title compound was obtained (10 mg, 18% yield) as a white solid by the same protocol as described in Step 3a above through hydrolysis of methyl 3-[[1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]bicyclo[1.1.1]pentane-1-carboxylate (product from Step 2b) (60 mg, 0.0988 mmol).

LC-MS (Method 1): Retention time=1.78 min. MS (ESI) m/z 592.0 (M−H)⁺.

¹H NMR (500 MHZ, CD₃OD) δ 8.22 (d, J=5.6 Hz, 1H), 7.35-7.26 (m, 2H), 7.24-7.20 (m, 1H), 7.18 (dd, J=6.9, 1.6 Hz, 1H), 7.14 (d, J=8.3 Hz, 1H), 6.26 (m, 1H), 4.91-4.86 (m, 1H), 2.76 (m, 1H), 2.54 (s, 1H), 2.37-2.06 (m, 7H), 1.90 (m, 2H), 1.75 (m, 1H), 1.66 (d, J=6.5 Hz, 3H).

### Examples 41 and 42

### 3-[[1-[3-[(1R)-1-(2,2-difluoro-1,3-benzodioxol-5-yl)ethoxy]phenyl]-3-(trifluoromethyl)-4,5,6,7-tetrahydroindazol-7-yl]oxy]bicyclo[1.1.1]pentane-1-carboxylic acid

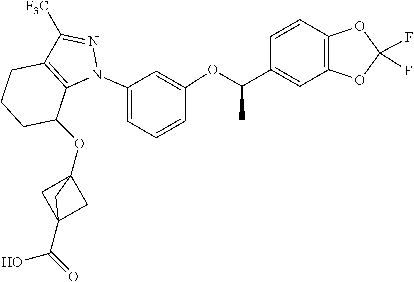 [SMILES: C[C@@H](Oc1cccc(-n2nc(C(F)(F)F)c3c2C(OC24CC(C(=O)O)(C2)C4)CCC3)c1)c1ccc2c(c1)OC(F)(F)O2]

### 3-[[1-[3-[(1S)-1-(2,2-difluoro-1,3-benzodioxol-5-yl)ethoxy]phenyl]-3-(trifluoromethyl)-4,5,6,7-tetrahydroindazol-7-yl]oxy]bicyclo[1.1.1]pentane-1-carboxylic acid

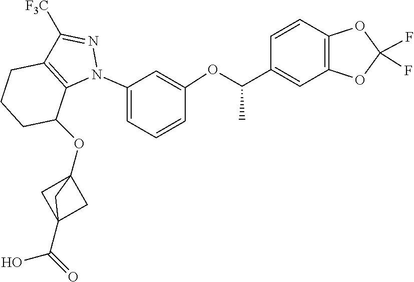 [SMILES: C[C@H](Oc1cccc(-n2nc(C(F)(F)F)c3c2C(OC24CC(C(=O)O)(C2)C4)CCC3)c1)c1ccc2c(c1)OC(F)(F)O2]

The title compound was synthesized essentially in the same manner as Examples 39 and 40.

### P1:

LC-MS (Method 2): Retention time=1.54 min. MS (ESI) m/z 593.2 [M+H]⁺.

¹H NMR (400 MHZ, CH₃OH-d₄) δ 7.42-7.21 (m, 3H), 7.19-6.99 (m, 4H), 5.53 (dq, J=12.8, 6.4 Hz, 1H), 4.67 (dt, J=51.7, 3.2 Hz, 1H), 2.80-2.71 (m, 1H), 2.51 (m, 1H), 2.12-1.68 (m, 10H), 1.63 (d, J=6.3 Hz, 3H).

### P2:

LC-MS (Method 2): Retention time=1.52 min. MS (ESI) m/z 593.2 [M+H]⁺.

¹H NMR (500 MHz, CH₃OH-d₄) δ 7.41-7.22 (m, 3H), 7.18-7.00 (m, 4H), 5.53 (dq, J=12.8, 6.3 Hz, 1H), 4.67 (dt, J=62.0, 4.0 Hz, 1H), 2.76 (d, J=15.5 Hz, 1H), 2.58-2.46 (m, 1H), 2.12-1.68 (m, 10H), 1.66-1.60 (m, 3H).

### Examples 43 and 44

### Trans-4-((1-(2-(R-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)cyclohexane-1-carboxylic acid

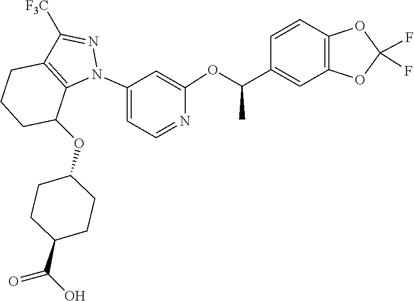 [SMILES: C[C@@H](Oc1cc(-n2nc(C(F)(F)F)c3c2C(O[C@H]2CC[C@H](C(=O)O)CC2)CCC3)ccn1)c1ccc2c(c1)OC(F)(F)O2]

### Trans-4-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)cyclohexane-1-carboxylic acid

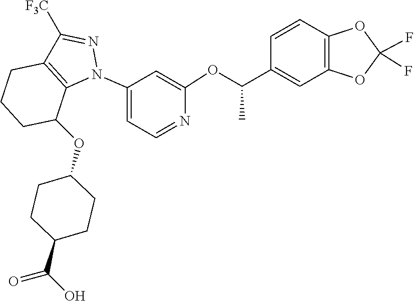 [SMILES: C[C@H](Oc1cc(-n2nc(C(F)(F)F)c3c2C(O[C@H]2CC[C@H](C(=O)O)CC2)CCC3)ccn1)c1ccc2c(c1)OC(F)(F)O2]

The title compound was synthesized essentially in the same manner as Examples 39 and 40.

P1: LC-MS (Method 1): Retention time=1.67 min. MS (ESI) m/z 610.2 (M+H)⁺.

¹H NMR (500 MHZ, CH₃OH-d₄) δ 8.21 (dd, J=5.9, 2.1 Hz, 1H), 7.32 (s, 1H), 7.29-7.21 (m, 3H), 7.15 (dd, J=8.3, 4.1 Hz, 1H), 6.25 (q, J=6.3 Hz, 1H), 4.89 (dd, J=8.8, 5.6 Hz, 1H), 3.52-3.35 (m, 1H), 2.75 (d, J=16.1 Hz, 1H), 2.62-2.43 (m, 1H), 2.21 (d, J=13.8 Hz, 1H), 2.11 (m, 1H), 2.02-1.68 (m, 7H), 1.69-1.57 (m, 3H), 1.54-1.14 (m, 3H), 1.14-0.92 (m, 1H).

P2: LC-MS (Method 1): Retention time=1.67 min. MS (ESI) m/z 610.2 (M+H)⁺.

¹H NMR (500 MHZ, CH₃OH-d₄) δ 8.21 (dd, J=5.8, 2.1 Hz, 1H), 7.33 (s, 1H), 7.29-7.20 (m, 3H), 7.15 (dd, J=8.2, 4.2 Hz, 1H), 6.26 (q, J=6.5 Hz, 1H), 4.90 (s, 1H), 3.55-3.38 (m, 1H), 2.75 (d, J=16.0 Hz, 1H), 2.55 (dd, J=19.5, 12.6 Hz, 1H), 2.20 (d, J=14.6 Hz, 1H), 2.11 (m, 1H), 1.82 (m, 7H), 1.65 (dd, J=6.5, 2.1 Hz, 3H), 1.55-1.14 (m, 3H), 1.17-0.87 (m, 1H).

### Examples 45 and 46

### 3-[[1-[2-[(1R)-1-(2,2-difluoro-[1,3]dioxolo[4,5-c]pyridine-6-yl)ethoxy]-4-pyridyl]-3-(trifluoromethyl)-4,5,6,7-tetrahydroindazol-7-yl]oxy]bicyclo[1.1.1]pentane-1-carboxylic acid

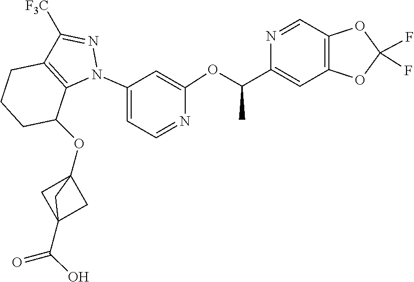 [SMILES: C[C@@H](Oc1cc(-n2nc(C(F)(F)F)c3c2C(OC24CC(C(=O)O)(C2)C4)CCC3)ccn1)c1cc2c(cn1)OC(F)(F)O2]

### 3-[[1-[2-[(1S)-1-(2,2-difluoro-[1,3]dioxolo[4,5-c]pyridine-6-yl)ethoxy]-4-pyridyl]-3-(trifluoromethyl)-4,5,6,7-tetrahydroindazol-7-yl]oxy]bicyclo[1.1.1]pentane-1-carboxylic acid

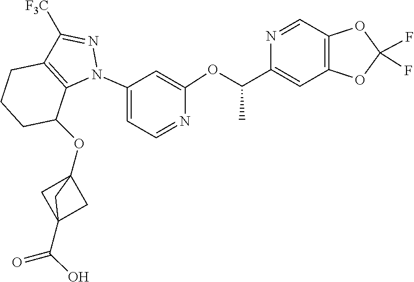 [SMILES: C[C@H](Oc1cc(-n2nc(C(F)(F)F)c3c2C(OC24CC(C(=O)O)(C2)C4)CCC3)ccn1)c1cc2c(cn1)OC(F)(F)O2]

The title compounds were synthesized essentially in the same manner as Examples 39 and 40.

P1: LCMS (Method 2): Retention time=1.47 min. MS (ESI) m/z 595.2 [M+H]⁺.

¹H NMR (400 MHZ, CH₃OH-d₄) δ:8.39 (s, 1H), 8.18 (d, J=5.3 Hz, 1H), 7.49 (d, J=3.6 Hz, 1H), 7.28-7.23 (m, 2H), 6.26-6.20 (m, 1H), 4.95-4.91 (m, 1H), 2.79-2.73 (m, 1H), 2.63-2.48 (m, 1H), 2.26-2.13 (m, 7H), 1.99-1.73 (m, 3H), 1.69 (d, J=6.6 Hz, 3H) ppm. (mixture of two diastereomers)

P2: LCMS (Method 2): Retention time=1.47 min. MS (ESI) m/z 595.2 [M+H]⁺.

¹H NMR (400 MHZ, CH₃OH-d₄) δ:8.38 (s, 1H), 8.23-8.10 (m, 1H), 7.49 (d, J=3.7 Hz, 1H), 7.29-2.22 (m, 2H), 6.31-6.20 (m, 1H), 4.95-4.91 (m, 1H), 2.79-2.72 (m, 1H), 2.60-2.51 (m, 1H), 2.26-2.09 (m, 7H), 2.05-1.71 (m, 3H), 1.69 (d, J=6.6 Hz, 3H) ppm.

### Examples 47 and 48

### Trans-4-((1-(2-(R-1-(2,2-difluoro-[1,3]dioxolo[4,5-c]pyridine-6-yl)ethoxy)pyridine-4-yl)-3-(trifluoromethyl)-4,5,6,7-tetrahydro-1H-indazol-7-yl)oxy)cyclohexane-1-carboxylic acid

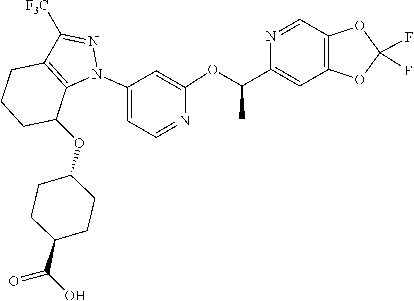 [SMILES: C[C@@H](Oc1cc(-n2nc(C(F)(F)F)c3c2C(O[C@H]2CC[C@H](C(=O)O)CC2)CCC3)ccn1)c1cc2c(cn1)OC(F)(F)O2]

### Trans-4-((1-(2-((S)-1-(2,2-difluoro-[1,3]dioxolo[4,5-c]pyridine-6-yl)ethoxy)pyridine-4-yl)-3-(trifluoromethyl)-4,5,6,7-tetrahydro-1H-indazol-7-yl)oxy)cyclohexane-1-carboxylic acid

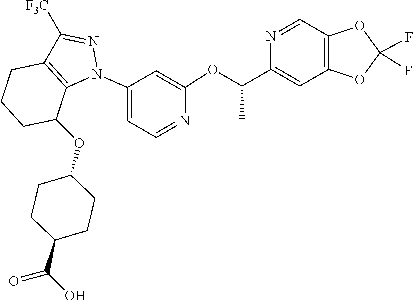 [SMILES: C[C@H](Oc1cc(-n2nc(C(F)(F)F)c3c2C(O[C@H]2CC[C@H](C(=O)O)CC2)CCC3)ccn1)c1cc2c(cn1)OC(F)(F)O2]

The title compounds were synthesized essentially in the same manner as Examples 39 and 40.

P1: LC-MS (Method 4): Retention time=1.98 min. MS (ESI) m/z 611.1 [M+H]⁺.

¹H NMR (400 MHZ, CH₃OH-d₄)) δ 8.43 (d, J=1.4 Hz, 1H), 8.22 (d, J=5.2 Hz, 1H), 7.52 (d, J=4.8 Hz, 1H), 7.36 (s, 1H), 7.30 (d, J=5.6 Hz, 1H), 6.32-6.23 (m, 1H), 4.94 (d, J=4.1 Hz, 1H), 3.50 (t, J=10.5 Hz, 1H), 2.83-2.76 (m, 1H), 2.64-2.57 (m, 1H), 2.30-2.09 (m, 2H), 2.05-1.77 (m, 7H), 1.71 (d, J=6.6 Hz, 3H), 1.54-1.27 (m, 3H), 1.12-1.00 (m, 1H).

P2: LC-MS (Method 4): Retention time=1.98 min. MS (ESI) m/z 611.1 [M+H]⁺.

¹H NMR (400 MHZ, CH₃OH-d₄) δ 8.43 (s, 1H), 8.22 (d, J=5.3 Hz, 1H), 7.52-3.46 (m, 1H), 7.36 (s, 1H), 7.30 (d, J=5.6 Hz, 1H), 6.35-6.22 (m, 1H), 5.00-4.91 (m, 1H), 3.50 (t, J=10.4 Hz, 1H), 2.79 (d, J=15.9 Hz, 1H), 2.69-2.52 (m, 1H), 2.32-2.09 (m, 2H), 2.06-1.76 (m, 7H), 1.71 (d, J=6.5 Hz, 3H), 1.58-1.42 (m, 1H), 1.39-1.28 (m, 2H), 1.11-1.00 (m, 1H).

### Examples 49 and 50

### 4-[[1-[5-[(1R)-1-(2,2-difluoro-1,3-benzodioxol-5-yl)ethoxy]-3-pyridyl]-3-(trifluoromethyl)-4,5,6,7-tetrahydroindazol-7-yl]oxy]benzoic acid

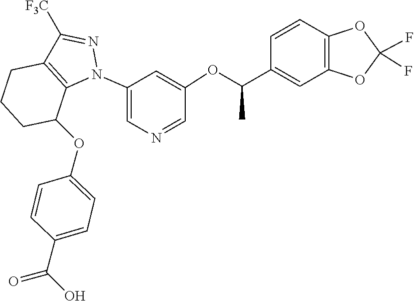 [SMILES: C[C@@H](Oc1cncc(-n2nc(C(F)(F)F)c3c2C(Oc2ccc(C(=O)O)cc2)CCC3)c1)c1ccc2c(c1)OC(F)(F)O2]

### 4-[[1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]oxy]benzoic acid

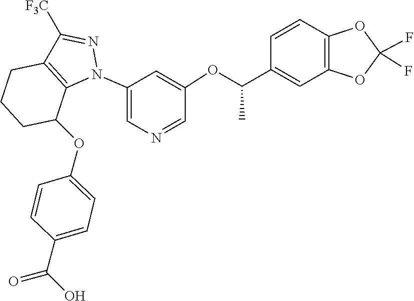 [SMILES: C[C@H](Oc1cncc(-n2nc(C(F)(F)F)c3c2C(Oc2ccc(C(=O)O)cc2)CCC3)c1)c1ccc2c(c1)OC(F)(F)O2]

The title compound was synthesized essentially in the same manner as Examples 39 and 40.

P1: LC-MS (Method 3): Retention time=2.28 min. MS (ESI) m/z 604.3 [M+H]⁺.

¹H NMR (500 MHZ, CH₃OH-d₄) δ 8.31 (dd, J=14.3, 2.0 Hz, 1H), 8.17 (dd, J=9.7, 2.6 Hz, 1H), 8.01-7.91 (m, 2H), 7.53 (dt, J=19.2, 2.3 Hz, 1H), 7.20-6.88 (m, 5H), 5.68 (dt, J=65.6, 3.0 Hz, 1H), 5.24 (p, J=6.3 Hz, 1H), 2.91-2.82 (m, 1H), 2.69-2.59 (m, 1H), 2.29-2.20 (m, 1H), 2.04-1.86 (m, 3H), 1.48 (dd, J=15.7, 6.3 Hz, 3H).

P2: LC-MS (Method 3): Retention time=2.28 min. MS (ESI) m/z 604.3 [M+H]⁺.

¹H NMR (500 MHZ, CH₃OH-d₄) δ 8.30 (dd, J=13.3, 1.7 Hz, 1H), 8.17 (dd, J=6.0, 2.4 Hz, 1H), 7.96 (dd, J=24.0, 8.7 Hz, 2H), 7.53 (dt, J=17.8, 2.1 Hz, 1H), 7.19-6.90 (m, 5H), 5.70 (d, J=66.6 Hz, 1H), 5.31-5.25 (m, 1H), 2.91-2.83 (m, 1H), 2.69-2.59 (m, 1H), 2.29-2.19 (m, 1H), 2.04-1.88 (m, 3H), 1.49 (dd, J=13.7, 6.3 Hz, 3H).

### Examples 51 and 52

### 3-((R-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylic acid

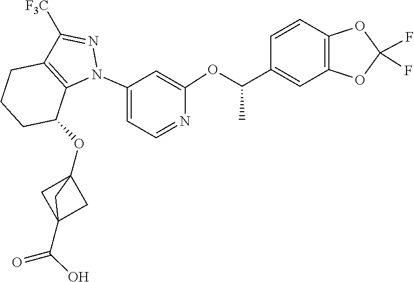 [SMILES: C[C@H](Oc1cc(-n2nc(C(F)(F)F)c3c2[C@H](OC24CC(C(=O)O)(C2)C4)CCC3)ccn1)c1ccc2c(c1)OC(F)(F)O2]

### 3-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylic acid

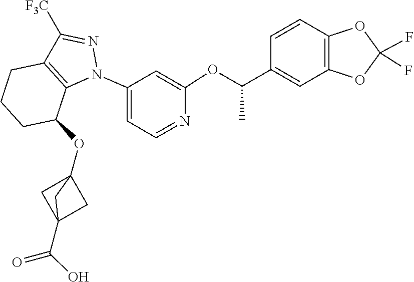 [SMILES: C[C@H](Oc1cc(-n2nc(C(F)(F)F)c3c2[C@@H](OC24CC(C(=O)O)(C2)C4)CCC3)ccn1)c1ccc2c(c1)OC(F)(F)O2]

### Step 1. Synthesis of methyl 3-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylate and methyl 3-(((R)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylate

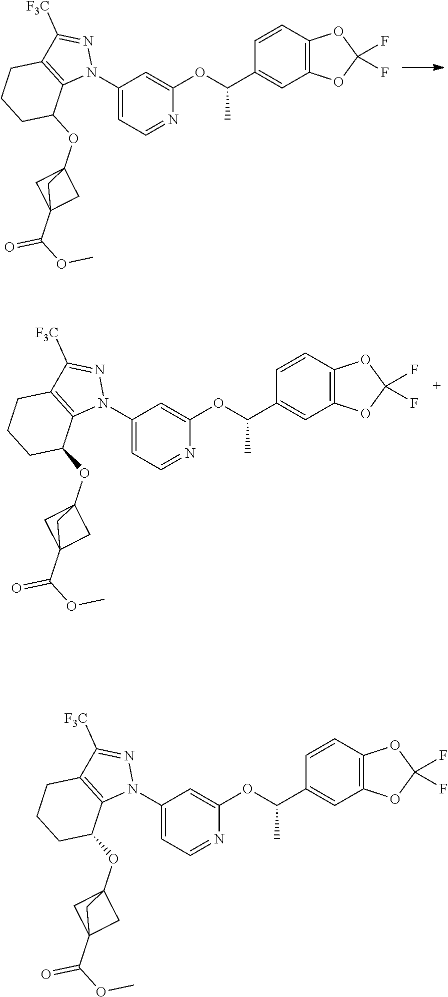 [SMILES: COC(=O)C12CC(OC3CCCc4c(C(F)(F)F)nn(-c5ccnc(O[C@@H](C)c6ccc7c(c6)OC(F)(F)O7)c5)c43)(C1)C2 ; COC(=O)C12CC(O[C@H]3CCCc4c(C(F)(F)F)nn(-c5ccnc(O[C@@H](C)c6ccc7c(c6)OC(F)(F)O7)c5)c43)(C1)C2 ; COC(=O)C12CC(O[C@@H]3CCCc4c(C(F)(F)F)nn(-c5ccnc(O[C@@H](C)c6ccc7c(c6)OC(F)(F)O7)c5)c43)(C1)C2]

The sample of methyl 3-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylate (from Examples 39 and 40, step 2b) (300 mg) was separated by chiral-HPLC (Column Name: OD-H 4.6×100 mm, 5 μm; Processing method: AS1 Acq. Method Set: 10% B3 Vial: 2: F, 3 Cosolvent: Isopropanol 0.5% NH₃ (7M in MeOH)] Injection volume: 5.00 μl. Channel name: PDA Ch2 254 nm @ 4.8 nm. Run time: 6.0 min. Processing channel description: PDA Ch2 254 nm @ 4.8 nm. Flow rate: 3.0 mL/min) to afford the title compounds with the first fraction designated as P1 (140 mg, 0.23 mmol) and the second fraction as P2 (120 mg, 0.20 mmol) as white solids for both compounds.

LC-MS (Method 2): Retention time=2.28 min. MS (ESI) m/z 423.8 [M-185+H]⁺.

### Step 2a. Synthesis of 3-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylic acid

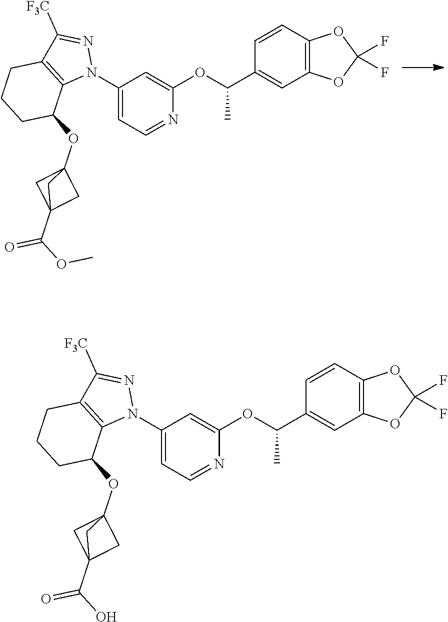 [SMILES: COC(=O)C12CC(O[C@H]3CCCc4c(C(F)(F)F)nn(-c5ccnc(O[C@@H](C)c6ccc7c(c6)OC(F)(F)O7)c5)c43)(C1)C2 ; C[C@H](Oc1cc(-n2nc(C(F)(F)F)c3c2[C@@H](OC24CC(C(=O)O)(C2)C4)CCC3)ccn1)c1ccc2c(c1)OC(F)(F)O2]

To a solution of methyl 3-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]bicyclo[1.1.1]pentane-1-carboxylate (P1 of the intermediate from Step 1) (140 mg, 0.23 mmol) in methanol (4 mL), THF (4 mL), and water (2 mL) was added lithium hydroxide monohydrate (48 mg, 1.15 mmol). The solution was stirred at rt for 8 h. Water (10 mL) was added to the reaction solution and the pH was adjusted to 5 with aqueous HCl (2 M), and then extracted with ethyl acetate (3×15 mL). The organic layer was concentrated. The crude product was purified by prep-HPLC (NH₄HCO₃ condition) to afford the title compound (115 mg, 84% yield) as a white solid.

LC-MS (Method 2): Retention time=1.78 min. MS (ESI) m/z 592.0 [M−H]⁺.

¹H NMR (400 MHZ, CH₃OH-d₄) δ 8.19 (d, J=5.6 Hz, 1H), 7.33-7.26 (m, 2H), 7.20 (dd, J=5.6, 1.8 Hz, 1H), 7.16 (d, J=1.6 Hz, 1H), 7.12 (d, J=8.3 Hz, 1H), 6.24 (q, J=6.6 Hz, 1H), 4.84 (d, J=3.3 Hz, 1H), 2.73 (d, J=16.5 Hz, 1H), 2.53 (m, 1H), 2.16 (m, 7H), 1.95-1.66 (m, 3H), 1.63 (d, J=6.5 Hz, 3H).

### Step 2b. Synthesis of 3-((R)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[1.1.1]pentane-1-carboxylic acid

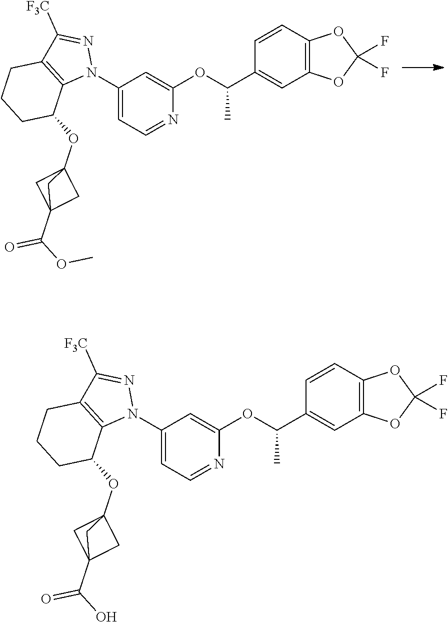 [SMILES: COC(=O)C12CC(O[C@@H]3CCCc4c(C(F)(F)F)nn(-c5ccnc(O[C@@H](C)c6ccc7c(c6)OC(F)(F)O7)c5)c43)(C1)C2 ; C[C@H](Oc1cc(-n2nc(C(F)(F)F)c3c2[C@H](OC24CC(C(=O)O)(C2)C4)CCC3)ccn1)c1ccc2c(c1)OC(F)(F)O2]

The title compound was obtained (101 mg, 84% yield) as a white solid by the same protocol as described in Step 2a through hydrolysis of methyl 3-[[(7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]bicyclo[1.1.1]pentane-1-carboxylate (P2 of the intermediate in Step 1) (120 mg, 0.20 mmol).

LC-MS (Method 1): Retention time=1.78 min. MS (ESI) m/z 592.0 [M−H]⁺.

¹H NMR (400 MHZ, CH₃OH-d₄) δ 8.19 (d, J=5.6 Hz, 1H), 7.31 (d, J=1.5 Hz, 1H), 7.26 (dd, J=8.4, 1.6 Hz, 1H), 7.23-7.16 (m, 2H), 7.12 (d, J=8.3 Hz, 1H), 6.22 (q, J=6.5 Hz, 1H), 4.86-4.82 (m, 1H), 2.74 (d, J=15.7 Hz, 1H), 2.53 (m, 1H), 2.18 (m, 7H), 1.97-1.66 (m, 3H), 1.63 (d, J=6.5 Hz, 3H).

### Examples 53 and 54

### Trans-4-(((R)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)cyclohexane-1-carboxylic acid

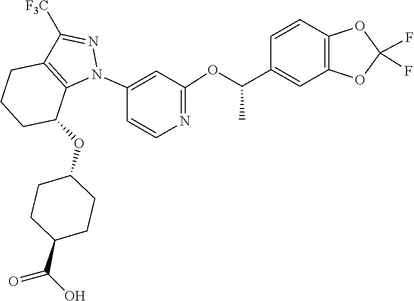 [SMILES: C[C@H](Oc1cc(-n2nc(C(F)(F)F)c3c2[C@H](O[C@H]2CC[C@H](C(=O)O)CC2)CCC3)ccn1)c1ccc2c(c1)OC(F)(F)O2]

### Trans-4-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)cyclohexane-1-carboxylic acid

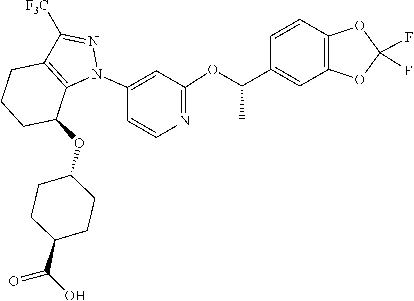 [SMILES: C[C@H](Oc1cc(-n2nc(C(F)(F)F)c3c2[C@@H](O[C@H]2CC[C@H](C(=O)O)CC2)CCC3)ccn1)c1ccc2c(c1)OC(F)(F)O2]

The title compounds were synthesized essentially in the same manner as Examples 51 and 52.

P1: LC-MS (Method 1): Retention time=1.88 min. MS (ESI) m/z 608.0 (M−H)⁺.

¹H NMR (500 MHZ, CH₃OH-d₄) δ 8.21 (d, J=5.5 Hz, 1H), 7.32 (s, 1H), 7.27 (d, J=7.9 Hz, 2H), 7.24 (dd, J=5.5, 1.5 Hz, 1H), 7.17 (d, J=8.3 Hz, 1H), 6.25 (q, J=6.5 Hz, 1H), 4.85 (d, J=3.3 Hz, 1H), 3.46 (t, J=10.6 Hz, 1H), 2.75 (d, J=16.6 Hz, 1H), 2.55 (ddd, J=16.3, 10.2, 5.8 Hz, 1H), 2.22 (d, J=14.1 Hz, 1H), 2.08 (t, J=11.8 Hz, 1H), 1.91 (t, J=25.9 Hz, 3H), 1.82 (d, J=9.5 Hz, 3H), 1.68 (dd, J=30.1, 10.1 Hz, 4H), 1.54-1.39 (m, 1H), 1.39-1.19 (m, 2H), 1.06 (dd, J=21.8, 11.0 Hz, 1H).

P2: LC-MS (Method 1): Retention time=1.89 min. MS (ESI) m/z 608.0 (M−H)⁺.

¹H NMR (500 MHZ, CH₃OH-d₄) δ 8.21 (d, J=5.3 Hz, 1H), 7.32 (s, 1H), 7.29-7.20 (m, 3H), 7.15 (d, J=8.3 Hz, 1H), 6.25 (q, J=6.5 Hz, 1H), 4.89 (d, J=1.5 Hz, 1H), 3.54-3.36 (m, 1H), 2.75 (d, J=16.1 Hz, 1H), 2.55 (ddd, J=16.4, 10.4, 5.9 Hz, 1H), 2.22 (d, J=14.2 Hz, 1H), 2.10 (t, J=11.8 Hz, 1H), 1.88 (dt, J=44.3, 16.0 Hz, 6H), 1.70 (dd, J=27.7, 14.7 Hz, 1H), 1.64 (d, J=6.5 Hz, 3H), 1.34 (tdd, J=22.1, 17.1, 10.7 Hz, 3H), 1.16-0.92 (m, 1H).

### Examples 55 and 56

### (1S,4r)-4-(((S)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)cyclohexane-1-carboxylic acid (P1)

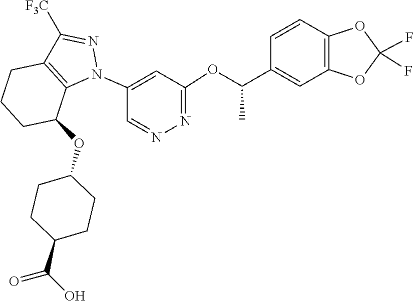 [SMILES: C[C@H](Oc1cc(-n2nc(C(F)(F)F)c3c2[C@@H](O[C@H]2CC[C@H](C(=O)O)CC2)CCC3)cnn1)c1ccc2c(c1)OC(F)(F)O2]

### (1R,4r)-4-(((R)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)cyclohexane-1-carboxylic acid (P2)

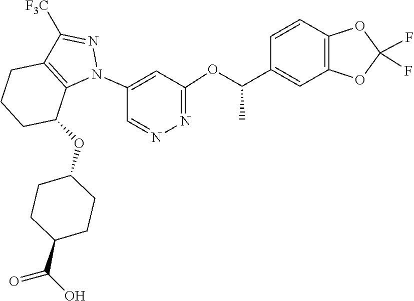 [SMILES: C[C@H](Oc1cc(-n2nc(C(F)(F)F)c3c2[C@H](O[C@H]2CC[C@H](C(=O)O)CC2)CCC3)cnn1)c1ccc2c(c1)OC(F)(F)O2]

### Step 1. Synthesis of(S)-1-(6-(1-(2,2-difluorobenzo[d][1,3]dioxol-5-yl)ethoxy)pyridazin-4-yl)-3-(trifluoromethyl)-1,4,5,6-tetrahydro-7H-indazol-7-one

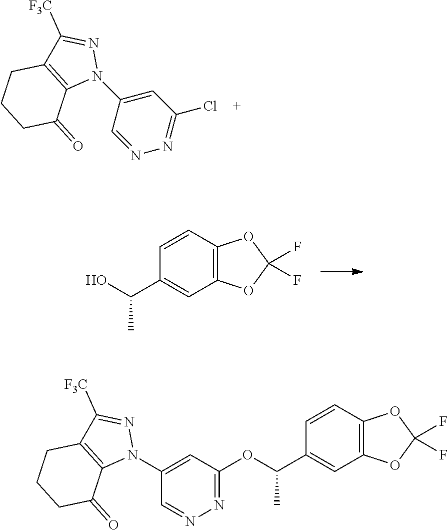 [SMILES: O=C1CCCc2c(C(F)(F)F)nn(-c3cnnc(Cl)c3)c21 ; C[C@H](O)c1ccc2c(c1)OC(F)(F)O2 ; C[C@H](Oc1cc(-n2nc(C(F)(F)F)c3c2C(=O)CCC3)cnn1)c1ccc2c(c1)OC(F)(F)O2]

To the solution of 1-(6-chloropyridazin-4-yl)-3-(trifluoromethyl)-5,6-dihydro-4H-indazol-7-one (Intermediate D-6) (1.35 g, 4.26 mmol) in dry toluene (20 mL) was added (1S)-1-(2,2-difluoro-1,3-benzodioxol-5-yl)ethanol (Intermediate C-1S) (948 mg, 4.69 mmol), 5-[bis(1,1-dimethylethyl)phosphino]-1′,3′,5′-triphenyl-1,4′-bi-1H-pyrazole (108 mg, 0.213 mmol), cesium carbonate (2778 mg, 8.53 mmol) and palladium (II) acetate (48 mg, 0.213 mmol). The solution was stirred at 90° C. for 4 h under N₂. The solution was concentrated, and the crude product was purified by flash column chromatography eluting with 15% ethyl acetate in PE, then further purified by SFC to get(S)-1-(6-(1-(2,2-difluorobenzo[d][1,3]dioxol-5-yl)ethoxy)pyridazin-4-yl)-3-(trifluoromethyl)-1,4,5,6-tetrahydro-7H-indazol-7-one (930 mg, 45%, ee >99%) as a white solid.

LC-MS (Method 2): Retention time=2.29 min. MS (ESI) m/z 483.1 [M+H]⁺.

### Step 2. Synthesis of 1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-ol

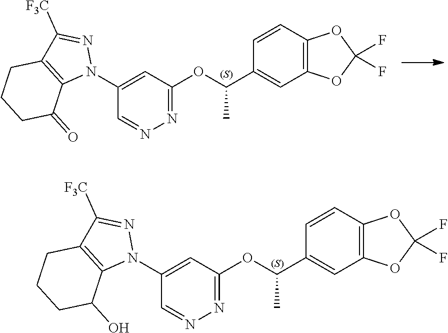

To the solution of(S)-1-(6-(1-(2,2-difluorobenzo[d][1,3]dioxol-5-yl)ethoxy)pyridazin-4-yl)-3-(trifluoromethyl)-1,4,5,6-tetrahydro-7H-indazol-7-one (640 mg, 1.33 mmol) in IPA (10 mL) was added triethylamine (4.30 mmol) and RuCl[(R,R)-Tsdpen](p-cymene) (Ref. 2) (3.0 mg, 0.00475 mmol), then formic acid (0.40 mL, 10.4 mmol) was added dropwise while the temperature was kept under 25° C. The reaction was stirred at 45° C. for 4 h. The reaction was concentrated, and the crude product was then purified by flash column chromatography eluted with 30% ethyl acetate in PE to get the title compound (585 mg, 91% yield) as a white solid.

LC-MS (Method 2): Retention time=2.26 min. MS (ESI) m/z 485.2 [M+H]⁺.

### Step 3. Synthesis of 7-chloro-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazole

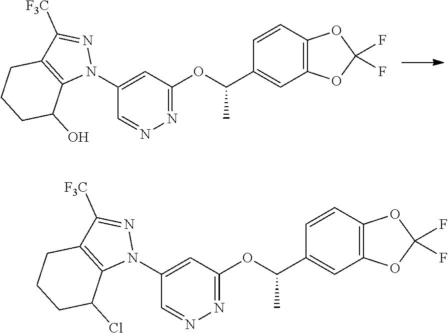 [SMILES: C[C@H](Oc1cc(-n2nc(C(F)(F)F)c3c2C(O)CCC3)cnn1)c1ccc2c(c1)OC(F)(F)O2 ; C[C@H](Oc1cc(-n2nc(C(F)(F)F)c3c2C(Cl)CCC3)cnn1)c1ccc2c(c1)OC(F)(F)O2]

To the solution of 1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-ol (600 mg, 1.24 mmol) in DCM (10 mL) was added methanesulfonyl chloride (0.24 mL, 3.10 mmol) and TEA (0.52 mL, 3.72 mmol) at 0° C. Then the solution was stirred at rt for 3 h. The reaction solution was concentrated. The crude product was purified by flash column chromatography eluted with 15% EA in PE to obtain the title compound (570 mg, 92% yield) as a white solid.

LC-MS (Method 2): Retention time=2.37 min. MS (ESI) m/z 503 [M+H]⁺.

### Step 4. Synthesis of ethyl (1S,4r)-4-(((S)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)cyclohexane-1-carboxylate and ethyl (1R,4r)-4-(((R)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)cyclohexane-1-carboxylate

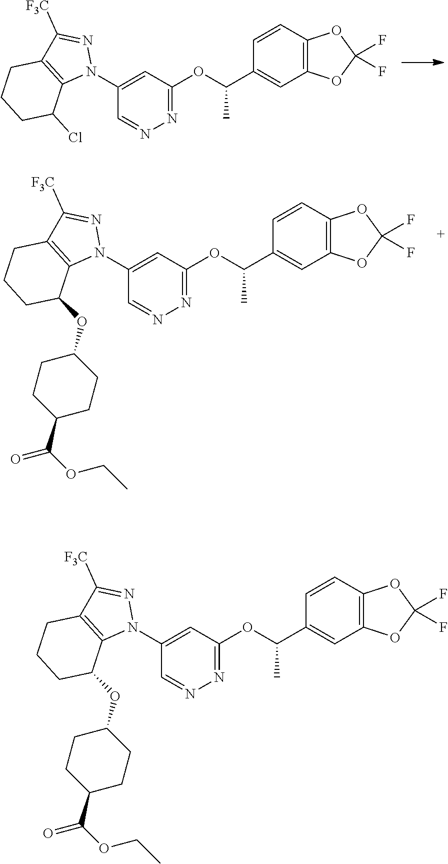 [SMILES: C[C@H](Oc1cc(-n2nc(C(F)(F)F)c3c2C(Cl)CCC3)cnn1)c1ccc2c(c1)OC(F)(F)O2 ; CCOC(=O)[C@H]1CC[C@H](O[C@H]2CCCc3c(C(F)(F)F)nn(-c4cnnc(O[C@@H](C)c5ccc6c(c5)OC(F)(F)O6)c4)c32)CC1 ; CCOC(=O)[C@H]1CC[C@H](O[C@@H]2CCCc3c(C(F)(F)F)nn(-c4cnnc(O[C@@H](C)c5ccc6c(c5)OC(F)(F)O6)c4)c32)CC1]

To the solution of 7-chloro-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazole (product of Step 3) (200 mg, 0.398 mmol) in DCM (10 mL) was added ethyl trans-4-hydroxycyclohexanecarboxylate (75 mg, 0.438 mmol), 2,6-di-tert-butylpyridine (228 mg, 1.19 mmol) and silver trifluoromethanesulfonate (123 mg, 0.477 mmol). The solution was stirred at rt for 3 h. The solution was concentrated, and the crude product was purified by flash column chromatography eluted with 15% EA in PE to get 100 mg of a white solid which was further purified by SFC to get the title compound with the first fraction designated as P1 (40 mg, 16% yield) and the second fraction designated as P2 (40 mg, 15% yield) as white solids for both compounds.

SFC method: IC 4.6×100 mm, 3 μm; MeOH [0.2% NH₃ (7M in MeOH)].

P1:

LC-MS (Method 2): Retention time=2.08 min. MS (ESI) m/z 661 [M+Na]⁺.

P2:

LC-MS (Method 2): Retention time=2.08 min. MS (ESI) m/z 661 [M+Na]⁺.

### Step 5a. Synthesis of (1S,4r)-4-(((S)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)cyclohexane-1-carboxylic acid

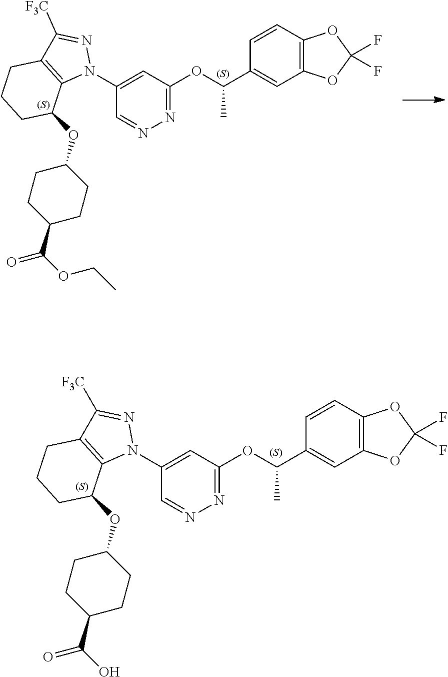

To the solution of ethyl (1S,4r)-4-(((S)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)cyclohexane-1-carboxylate (the intermediate P1 in Step 4) (40 mg, 0.0626 mmol) in methanol (1 mL) and THF (1 mL) was added aqueous LiOH (2 M, 1 mL) and the solution was stirred at rt for 4 h. The pH of the solution was adjusted to 6. The crude product was purified by prep-HPLC to get the title compound (22 mg, 58% yield) as a white solid.

LC-MS (Method 1): Retention time=1.86 min. MS (ESI) m/z 611 [M+H]⁺.

¹H NMR (500 MHZ, CH₃OH-d₄) δ 9.24 (d, J=2.0 Hz, 1H), 7.71 (d, J=2.0 Hz, 1H), 7.38 (d, J=1.4 Hz, 1H), 7.32 (dd, J=8.3, 1.4 Hz, 1H), 7.20 (d, J=8.3 Hz, 1H), 6.42 (q, J=6.5 Hz, 1H), 4.99 (t, J=3.4 Hz, 1H), 3.65-3.54 (m, 1H), 2.83-2.70 (m, 1H), 2.62-2.51 (m, 1H), 2.27 (dd, J=11.3, 5.7 Hz, 1H), 2.15-2.01 (m, 2H), 2.01-1.89 (m, 4H), 1.88-1.66 (m, 5H), 1.56-1.41 (m, 2H), 1.37-1.23 (m, 1H), 1.23-1.10 (m, 1H).

### Step 5b. Synthesis of (1R,4r)-4-(((R)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)cyclohexane-1-carboxylic acid

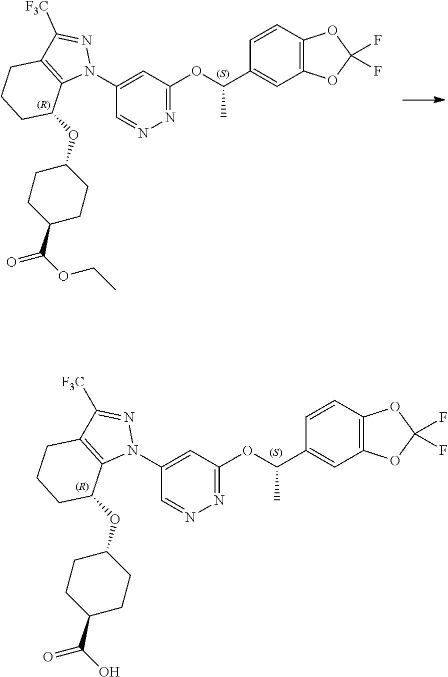

The title compound was obtained (21 mg, 55% yield) as a white solid by the same protocol as described in Step 5a through hydrolysis of ethyl (1R,4r)-4-(((R)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)cyclohexane-1-carboxylate (the intermediate P2 from Step 4) (40 mg, 0.0626 mmol).

LC-MS (Method 1): Retention time=1.81 min. MS (ESI) m/z 611 [M+H]⁺.

¹H NMR (500 MHZ, CH₃OH-d₄) δ 9.21 (d, J=1.9 Hz, 1H), 7.73 (d, J=1.9 Hz, 1H), 7.38 (s, 1H), 7.34 (dd, J=8.4, 1.2 Hz, 1H), 7.20 (d, J=8.3 Hz, 1H), 6.45 (q, J=6.4 Hz, 1H), 4.97 (t, J=3.3 Hz, 1H), 3.62-3.55 (m, 1H), 2.80-2.71 (m, 1H), 2.62-2.52 (m, 1H), 2.29-2.20 (m, 1H), 2.13-2.01 (m, 2H), 2.00-1.88 (m, 4H), 1.88-1.69 (m, 5H), 1.56-1.41 (m, 2H), 1.34-1.23 (m, 1H), 1.21-1.11 (m, 1H).

### Examples 57 and 58

### 4—[[(7S)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]oxy]bicyclo[2.2.2]octane-1-carboxylic acid (P1)

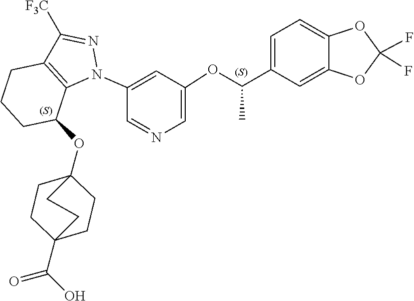

### 4-[[(7R)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]oxy]bicyclo[2.2.2]octane-1-carboxylic acid (P2)

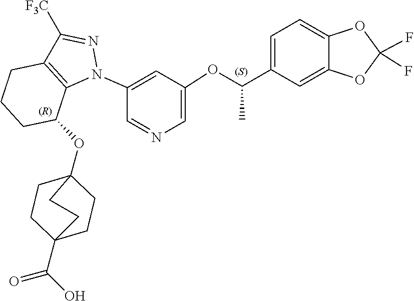

The title compounds were synthesized by following the procedures in Examples 55 and 56 to obtain the final acid as the diastereomeric mixture (90 mg, 0.142 mmol) which was separated by chiral-HPLC to give the title compound with the first fraction designated as P1 (27 mg, 30% yield; a white solid) and the second fraction designated as P2 (26 mg, 29% yield; a white solid).

P1: Chiral Method: Column Name: IH 4.6×100 mm 5 μm. Solvent: IPA [0.2% NH₃ (7M in MeOH)]; Retention time=2.334 min; ee=100%.

LC-MS (Method 4): Retention time=1.91 min. MS (ESI) m/z 636.2 [M+H]⁺.

¹H NMR (500 MHZ, CH₃OH-d₄) δ 8.40 (d, J=2.4 Hz, 1H), 8.30 (d, J=1.5 Hz, 1H), 7.52 (t, J=2.2 Hz, 1H), 7.36 (d, J=1.5 Hz, 1H), 7.28 (dd, J=8.3, 1.5 Hz, 1H), 7.19 (d, J=8.3 Hz, 1H), 5.67 (q, J=6.3 Hz, 1H), 4.98 (t, J=3.7 Hz, 1H), 2.70 (d, J=15.9 Hz, 1H), 2.53 (dt, J=14.9, 7.6 Hz, 1H), 2.00-1.85 (m, 3H), 1.81-1.74 (m, 1H), 1.69 (dd, J=10.0, 7.2 Hz, 9H), 1.26 (ddd, J=15.9, 8.6, 3.2 Hz, 3H), 1.15 (ddd, J=15.2, 10.1, 3.1 Hz, 3H).

P2: Chiral Method: Column Name: IH 4.6×100 mm 5 μm. Solvent: IPA [0.2% NH₃ (7M in MeOH)]; Retention time=3.310 min; ee=99.02%.

LC-MS (Method 4): Retention time=1.91 min. MS (ESI) m/z 636.2 [M+H]⁺.

¹H NMR (500 MHZ, CH₃OH-d₄) δ 8.40 (s, 1H), 8.29 (s, 1H), 7.56 (t, J=2.1 Hz, 1H), 7.42 (d, J=1.3 Hz, 1H), 7.34 (dd, J=8.3, 1.4 Hz, 1H), 7.25 (d, J=8.3 Hz, 1H), 5.63 (q, J=6.3 Hz, 1H), 4.62 (d, J=3.7 Hz, 1H), 2.71 (d, J=15.9 Hz, 1H), 2.59-2.47 (m, 1H), 1.98-1.73 (m, 4H), 1.68 (dd, J=11.0, 4.8 Hz, 9H), 1.22 (dt, J=10.6, 5.9 Hz, 3H), 1.12 (dt, J=12.2, 5.5 Hz, 3H).

### Examples 59 and 60

### 4-(((S)-1-(5-((S)-1-(2,2-difluorobenzo[d][1,3]dioxol-5-yl)ethoxy)-6-fluoropyridin-3-yl)-3-(trifluoromethyl)-4,5,6,7-tetrahydro-1H-indazol-7-yl)oxy)bicyclo[2.2.2]octane-1-carboxylic acid (P1)

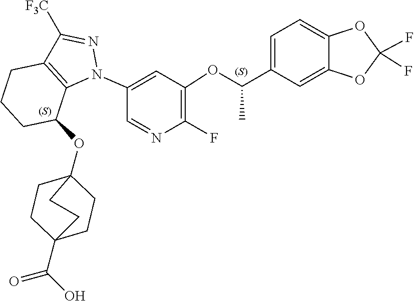

### 4-(((R)-1-(5-((S)-1-(2,2-difluorobenzo[d][1,3]dioxol-5-yl)ethoxy)-6-fluoropyridin-3-yl)-3-(trifluoromethyl)-4,5,6,7-tetrahydro-1H-indazol-7-yl)oxy)bicyclo[2.2.2]octane-1-carboxylic acid (P2)

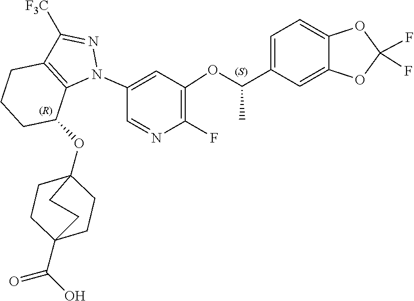

The title compounds were synthesized essentially in the same manner as Examples 55 and 56.

P1: LC-MS (Method 2): Retention time=1.87 min. MS (ESI) m/z 654.1 [M+H]⁺.

¹H NMR (400 MHZ, CH₃OH-d₄) δ 7.89 (t, J=1.9 Hz, 1H), 7.56 (dd, J=8.7, 2.1 Hz, 1H), 7.34 (d, J=1.4 Hz, 1H), 7.27 (dd, J=8.3, 1.5 Hz, 1H), 7.19 (d, J=8.3 Hz, 1H), 5.67 (q, J=6.3 Hz, 1H), 4.93 (d, J=3.6 Hz, 1H), 2.68 (d, J=15.5 Hz, 1H), 2.50 (dd, J=15.2, 7.8 Hz, 1H), 1.96-1.82 (m, 3H), 1.79-1.61 (m, 10H), 1.25-1.13 (m, 3H), 1.12-0.99 (m, 3H).

P2: LC-MS (Method 2): Retention time=1.86 min. MS (ESI) m/z 654.2 [M+H]⁺.

¹H NMR (400 MHZ, CH₃OH-d₄) δ 7.86 (t, J=1.9 Hz, 1H), 7.63 (dd, J=8.7, 2.1 Hz, 1H), 7.44 (d, J=1.4 Hz, 1H), 7.35 (dd, J=8.3, 1.5 Hz, 1H), 7.29 (d, J=8.3 Hz, 1H), 5.60 (q, J=6.3 Hz, 1H), 4.43 (d, J=3.2 Hz, 1H), 2.70 (d, J=15.4 Hz, 1H), 2.59-2.40 (m, 1H), 1.97-1.85 (m, 2H), 1.81-1.64 (m, 11H), 1.16 (m, 3H), 1.04 (m, 3H).

### Example 61

### 4-(((S)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)benzoic acid

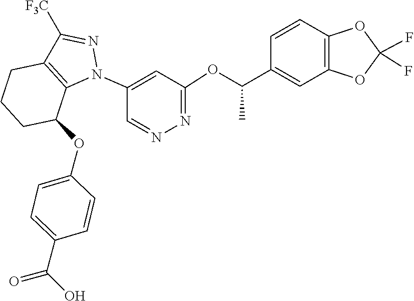 [SMILES: C[C@H](Oc1cc(-n2nc(C(F)(F)F)c3c2[C@@H](Oc2ccc(C(=O)O)cc2)CCC3)cnn1)c1ccc2c(c1)OC(F)(F)O2]

The title compound was synthesized essentially in the same manner as Examples 55 and 56.

LC-MS (Method 1): Retention time=1.86 min. MS (ESI) m/z 605.0 [M+H]⁺.

¹H NMR (400 MHZ, CH₃OH-d₄) δ 9.15 (d, J=2.1 Hz, 1H), 8.01 (d, J=8.8 Hz, 2H), 7.32 (d, J=2.1 Hz, 1H), 7.17 (m, 2H), 7.02 (m, 3H), 6.17 (q, J=6.4 Hz, 1H), 5.79 (s, 1H), 2.87 (d, J=15.9 Hz, 1H), 2.73-2.56 (m, 1H), 2.37-2.23 (m, 1H), 2.07-1.93 (m, 3H), 1.52 (d, J=6.5 Hz, 3H).

### Examples 62 and 63

### 3-(((S)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)bicyclo[1.1.1]pentane-1-carboxylic acid (P1)

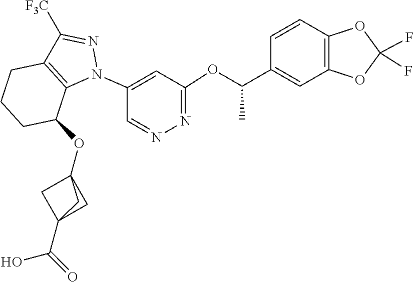 [SMILES: C[C@H](Oc1cc(-n2nc(C(F)(F)F)c3c2[C@@H](OC24CC(C(=O)O)(C2)C4)CCC3)cnn1)c1ccc2c(c1)OC(F)(F)O2]

### 3-(((R)-1-(6-((S)-1-(2,2-difluorobenzo[d][1,3]dioxol-5-yl)ethoxy)pyridazin-4-yl)-3-(trifluoromethyl)-4,5,6,7-tetrahydro-1H-indazol-7-yl)oxy)bicyclo[1.1.1]pentane-1-carboxylic acid (P2)

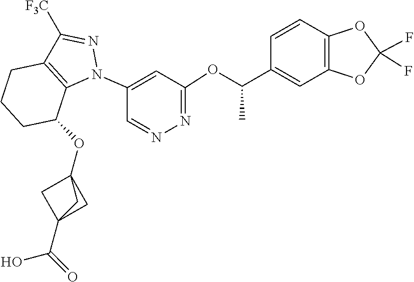 [SMILES: C[C@H](Oc1cc(-n2nc(C(F)(F)F)c3c2[C@H](OC24CC(C(=O)O)(C2)C4)CCC3)cnn1)c1ccc2c(c1)OC(F)(F)O2]

The title compound was synthesized essentially in the same manner as Examples 55 and 56.

P1: LC-MS (Method 1): Retention time=1.85 min. MS (ESI) m/z 595.1 [M+H]⁺.

SFC condition: IH 4.6*100 mm, 5 μm; rt=1.96 min

¹H NMR (500 MHZ, CH₃OH-d₄) δ 9.19 (d, J=2.0 Hz, 1H), 7.55 (d, J=2.0 Hz, 1H), 7.41 (s, 1H), 7.35 (dd, J=8.3, 1.3 Hz, 1H), 7.17 (d, J=8.3 Hz, 1H), 6.43 (q, J=6.5 Hz, 1H), 5.00 (t, J=3.6 Hz, 1H), 2.84-2.68 (m, 1H), 2.64-2.50 (m, 1H), 2.31-2.12 (m, 7H), 2.02-1.78 (m, 3H), 1.75 (d, J=6.5 Hz, 3H).

P2: LC-MS (Method 1): Retention time=1.85 min. MS (ESI) m/z 595.1 [M+H]⁺.

SFC condition: IH 4.6×100 mm, 5 μm; Retention time=1.53 min

¹H NMR (500 MHZ, CH₃OH-d₄) δ 9.19 (d, J=1.8 Hz, 1H), 7.55 (d, J=1.9 Hz, 1H), 7.40 (s, 1H), 7.37 (d, J=8.4 Hz, 1H), 7.18 (d, J=8.3 Hz, 1H), 6.45 (q, J=6.5 Hz, 1H), 4.97 (t, J=3.5 Hz, 1H), 2.80-2.71 (m, 1H), 2.60-2.50 (m, 1H), 2.31-2.12 (m, 7H), 2.00-1.76 (m, 3H), 1.75 (d, J=6.5 Hz, 3H).

### Examples 64 and 65

### 4-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[2.2.2]octane-1-carboxylic acid

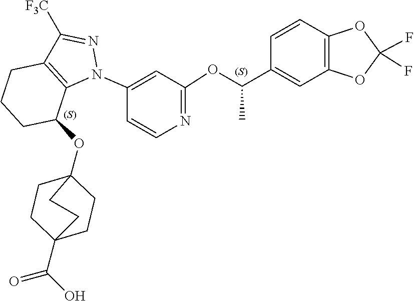

### 4-(((R)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)oxy)bicyclo[2.2.2]octane-1-carboxylic acid

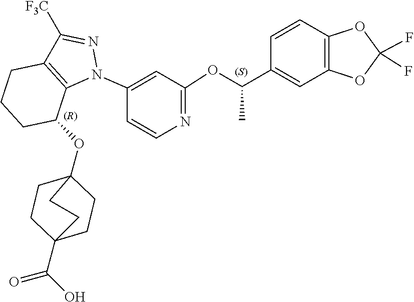

The title compound was synthesized essentially in the same manner as Examples 55 and 56.

P1: LC-MS (Method 1): Retention time=1.93 min. MS (ESI) m/z 634.0 (M−H)⁺.

¹H NMR (500 MHZ, CH₃OH-d₄) δ 8.22 (d, J=5.5 Hz, 1H), 7.33 (s, 1H), 7.28 (dd, J=8.3, 1.2 Hz, 1H), 7.21 (s, 1H), 7.20-7.14 (m, 2H), 6.26 (q, J=6.4 Hz, 1H), 5.10-4.96 (m, 1H), 2.72 (d, J=16.0 Hz, 1H), 2.53 (ddd, J=15.8, 9.7, 5.8 Hz, 1H), 2.18-2.04 (m, 1H), 1.95 (d, J=13.0 Hz, 1H), 1.81 (t, J=8.0 Hz, 8H), 1.65 (t, J=11.5 Hz, 3H), 1.62-1.41 (m, 6H).

P2: LC-MS (Method 1): Retention time=1.96 min. MS (ESI) m/z 634.0 (M−H)⁺.

¹H NMR (500 MHZ, CH₃OH-d₄) δ 8.22 (d, J=5.5 Hz, 1H), 7.33 (s, 1H), 7.27 (d, J=8.4 Hz, 1H), 7.23-7.14 (m, 3H), 6.25 (q, J=6.5 Hz, 1H), 5.03 (d, J=27.7 Hz, 1H), 2.73 (d, J=15.5 Hz, 1H), 2.54 (dt, J=22.3, 8.0 Hz, 1H), 2.14 (d, J=11.7 Hz, 1H), 2.07-1.87 (m, 1H), 1.89-1.71 (m, 8H), 1.71-1.38 (m, 9H).

### Examples 66 and 67

### Trans-4-[[(7S)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]oxy]cyclohexanecarboxylic acid (P1)

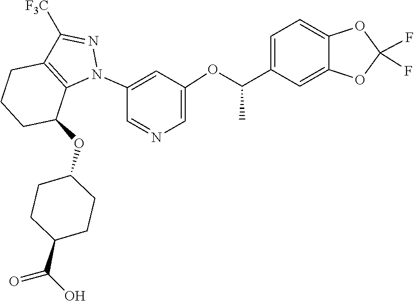 [SMILES: C[C@H](Oc1cncc(-n2nc(C(F)(F)F)c3c2[C@@H](O[C@H]2CC[C@H](C(=O)O)CC2)CCC3)c1)c1ccc2c(c1)OC(F)(F)O2]

### Trans-4-[[(7R)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]oxy]cyclohexanecarboxylic acid (P2)

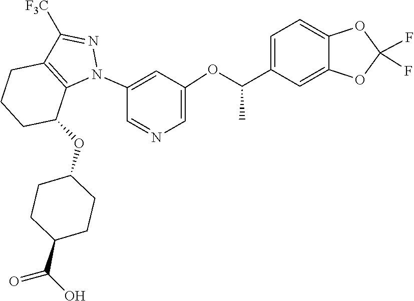 [SMILES: C[C@H](Oc1cncc(-n2nc(C(F)(F)F)c3c2[C@H](O[C@H]2CC[C@H](C(=O)O)CC2)CCC3)c1)c1ccc2c(c1)OC(F)(F)O2]

The title compound was synthesized essentially in the same manner as Examples 55 and 56.

P1: Chiral Method: Column Name: IH 4.6×100 mm 5 μm. Solvent: MeOH [0.1% NH₃ 7M in MeOH)]; Retention time=1.934 min; ee=99.96%.

LC-MS (Method 4): Retention time=1.88 min. MS (ESI) m/z 610.1 [M+H]⁺.

¹H NMR (500 MHZ, CH₃OH-d₄) δ 8.38 (s, 2H), 7.61 (t, J=2.1 Hz, 1H), 7.35 (d, J=1.3 Hz, 1H), 7.28 (dd, J=8.3, 1.3 Hz, 1H), 7.18 (d, J=8.3 Hz, 1H), 5.66 (q, J=6.3 Hz, 1H), 4.78 (t, J=3.5 Hz, 1H), 3.26-3.18 (m, 1H), 2.73 (d, J=16.2 Hz, 1H), 2.60-2.49 (m, 1H), 2.12-1.98 (m, 2H), 1.92-1.76 (m, 5H), 1.75-1.63 (m, 4H), 1.43-1.29 (m, 2H), 1.21 (qd, J=13.1, 3.4 Hz, 1H), 1.11-0.99 (m, 1H), 0.57 (qd, J=12.8, 3.6 Hz, 1H).

P2: Chiral Method: Column Name: IH 4.6×100 mm 5 μm. Solvent: MeOH [0.1% NH₃ (7M in MeOH)]; Retention time=2.344 min; ee=99.12%.

LC-MS (Method 4): Retention time=1.87 min. MS (ESI) m/z 610.1 [M+H]⁺.

¹H NMR (500 MHZ, CH₃OH-d₄) δ 8.37 (d, J=18.3 Hz, 2H), 7.60 (s, 1H), 7.39 (d, J=1.3 Hz, 1H), 7.31 (dd, J=8.3, 1.2 Hz, 1H), 7.22 (d, J=8.3 Hz, 1H), 5.63 (q, J=6.3 Hz, 1H), 4.45 (t, J=3.4 Hz, 1H), 3.08-2.97 (m, 1H), 2.74 (d, J=16.2 Hz, 1H), 2.54 (ddd, J=16.2, 10.2, 5.9 Hz, 1H), 2.10-2.02 (m, 2H), 1.92-1.64 (m, 9H), 1.32 (ddd, J=15.4, 13.2, 3.3 Hz, 1H), 1.24-1.06 (m, 3H), 0.67-0.55 (m, 1H).

### Example 68

### 4-(((S)-1-(5-((S)-1-(2,2-difluorobenzo[d][1,3]dioxol-5-yl)ethoxy)-6-fluoropyridin-3-yl)-3-(trifluoromethyl)-4,5,6,7-tetrahydro-1H-indazol-7-yl)oxy)benzoic acid

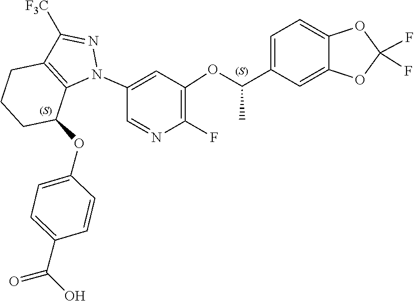

### Step 1. Synthesis of(S)-5-bromo-3-(1-(2,2-difluorobenzo[d][1,3]dioxol-5-yl)ethoxy)-2-fluoropyridine

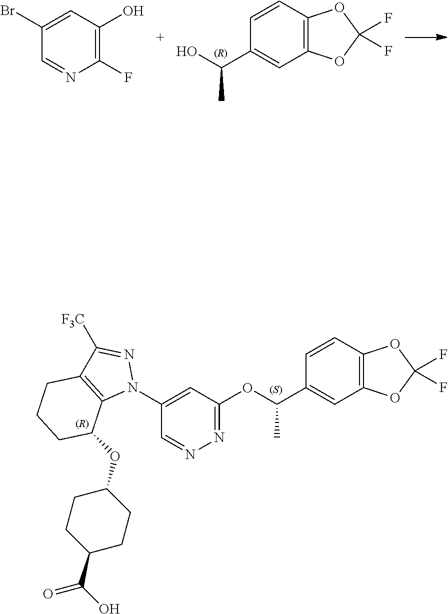

To a solution of 5-bromo-2-fluoro-pyridin-3-ol (700 mg, 3.65 mmol) and (1R)-1-(2,2-difluoro-1,3-benzodioxol-5-yl)ethanol (Intermediate C-1R) (884 mg, 4.38 mmol) in THF (15 mL), was added PPh₃ (2869 mg, 10.9 mmol) and DIAD (0.87 mL, 5.47 mmol) at 0° C. The solution was stirred at room temperature for 1 h. The reaction solution was concentrated in vacuo to give a residue, which was purified by flash silica gel column chromatography (ethyl acetate in petroleum ether from 0% to 15% yield) to afford the title compound (1400 mg, 97% yield) as a yellow oil.

LC-MS (Method 3): Retention time=1.88 min. MS (ESI) m/z 376/378.0 [M+H]⁺.

### Step 2. Synthesis of(S)-(5-(1-(2,2-difluorobenzo[d][1,3]dioxol-5-yl)ethoxy)-6-fluoropyridin-3-yl) boronic acid

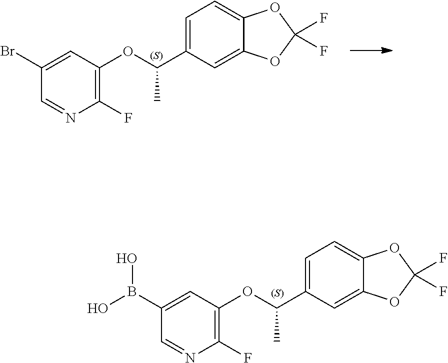

To a solution of 5-bromo-3-[(1S)-1-(2,2-difluoro-1,3-benzodioxol-5-yl)ethoxy]-2-fluoro-pyridine (1300 mg, 3.46 mmol) in 1,4-dioxane (20 mL) was added bis(pinacolato)diboron (1053 mg, 4.15 mmol), potassium acetate (678 mg, 6.91 mmol), and Pd (dppf)₂Cl₂ (130 mg). The solution was stirred at 80° C. for 8 h. The reaction solution was concentrated in vacuo to give a residue which was purified by flash silica gel column chromatography (ethyl acetate in petroleum ether from 0% to 12%) to afford a crude product which was then purified by prep-HPLC to afford the title compound (1000 mg, 85% yield) as a yellow oil.

LC-MS (Method 1): Retention time=1.95 min. MS (ESI) m/z 342.0 [M+H]⁺.

### Step 3. Synthesis of(S)-1-(5-(1-(2,2-difluorobenzo[d][1,3]dioxol-5-yl)ethoxy)-6-fluoropyridin-3-yl)-3-(trifluoromethyl)-1,4,5,6-tetrahydro-7H-indazol-7-one

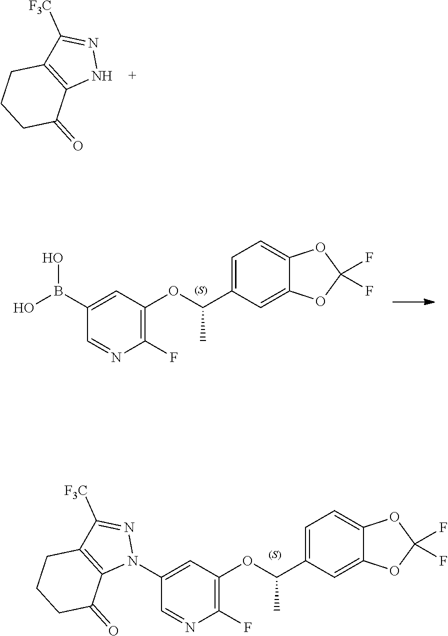

To a solution of [5-[(1S)-1-(2,2-difluoro-1,3-benzodioxol-5-yl)ethoxy]-6-fluoro-3-pyridyl]boronic acid (550 mg, 1.61 mmol) in DCE (15 mL) was added 3-(trifluoromethyl)-1,4,5,6-tetrahydroindazol-7-one (Intermediate D-1) (329 mg, 1.61 mmol), copper (II) acetate (146 mg, 0.806 mmol), and triethylamine (0.25 mL, 1.77 mmol). The solution was stirred at rt for 8 h. The reaction solution was concentrated in vacuo to give a residue which was purified by prep-HPLC (NH₄HCO₃ modified) to afford the crude product which was then purified by SFC to afford the title compound (450 mg, 56% yield) as a brown oil.

LC-MS (Method 4): Retention time=2.34 min. MS (ESI) m/z 500.1 [M+H]⁺.

### Step 4. Synthesis of R-1-(5-((S)-1-(2,2-difluorobenzo[d][1,3]dioxol-5-yl)ethoxy)-6-fluoropyridin-3-yl)-3-(trifluoromethyl)-4,5,6,7-tetrahydro-1H-indazol-7-ol

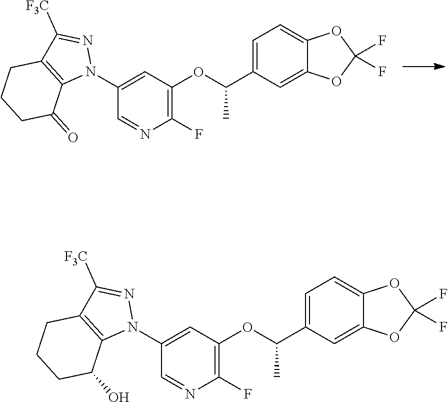 [SMILES: C[C@H](Oc1cc(-n2nc(C(F)(F)F)c3c2C(=O)CCC3)cnc1F)c1ccc2c(c1)OC(F)(F)O2 ; C[C@H](Oc1cc(-n2nc(C(F)(F)F)c3c2[C@H](O)CCC3)cnc1F)c1ccc2c(c1)OC(F)(F)O2]

To a solution of 1-[5-[(1S)-1-(2,2-difluoro-1,3-benzodioxol-5-yl)ethoxy]-6-fluoro-3-pyridyl]-3-(trifluoromethyl)-5,6-dihydro-4H-indazol-7-one (70 mg, 0.140 mmol) and bis(dichloro(n6-p-cymene)ruthenium) (1R,2R)—N-p-toluenesulfonyl-1,2-diphenylethylenediamine (5 mg) in IPA (5 mL) under N₂, and then was added triethylamine (0.30 mL, 2.15 mmol) and formic acid (0.20 mL, 5.21 mmol) while the temperature was kept below 45° C. under N₂. The solution was stirred at 45° C. for 4 h. The reaction solution was concentrated in vacuo to give a residue which was purified by flash silica gel column chromatography (ethyl acetate in petroleum ether from 0% to 20%) to afford the title compound (60 mg, 86% yield) as a light-yellow oil.

LC-MS (Method 2): Retention time=2.05 min. MS (ESI) m/z 501.8 [M+H]⁺.

### Step 5. Synthesis of methyl 4-(((S)-1-(5-((S)-1-(2,2-difluorobenzo[d][1,3]dioxol-5-yl)ethoxy)-6-fluoropyridin-3-yl)-3-(trifluoromethyl)-4,5,6,7-tetrahydro-1H-indazol-7-yl)oxy)benzoate

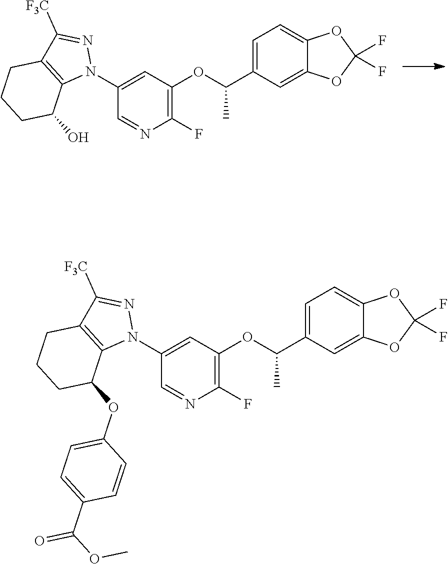 [SMILES: C[C@H](Oc1cc(-n2nc(C(F)(F)F)c3c2[C@H](O)CCC3)cnc1F)c1ccc2c(c1)OC(F)(F)O2 ; COC(=O)c1ccc(O[C@H]2CCCc3c(C(F)(F)F)nn(-c4cnc(F)c(O[C@@H](C)c5ccc6c(c5)OC(F)(F)O6)c4)c32)cc1]

To a solution of (7R)-1-[5-[(1S)-1-(2,2-difluoro-1,3-benzodioxol-5-yl)ethoxy]-6-fluoro-3-pyridyl]-3-(trifluoromethyl)-4,5,6,7-tetrahydroindazol-7-ol (60 mg, 0.120 mmol) and methyl 4-hydroxybenzoate (22 mg, 0.144 mmol) in THF (5 mL) at 0° C. were added triphenylphosphine (63 mg, 0.239 mmol) and diethyl azodicarboxylate (0.028 mL, 0.180 mmol). The solution was stirred at rt for 1 h. The reaction solution was concentrated in vacuo to give a residue which was purified by prep-HPLC (NH₄HCO₃ modified) to afford the title compound (40 mg, 53% yield) as a white solid.

LC-MS (Method 2): Retention time=2.60 min. MS (ESI) m/z 452.1 [M-185+H]⁺.

### Step 6. Synthesis of 4-(((S)-1-(5-((S)-1-(2,2-difluorobenzo[d][1,3]dioxol-5-yl)ethoxy)-6-fluoropyridin-3-yl)-3-(trifluoromethyl)-4,5,6,7-tetrahydro-1H-indazol-7-yl)oxy)benzoic acid

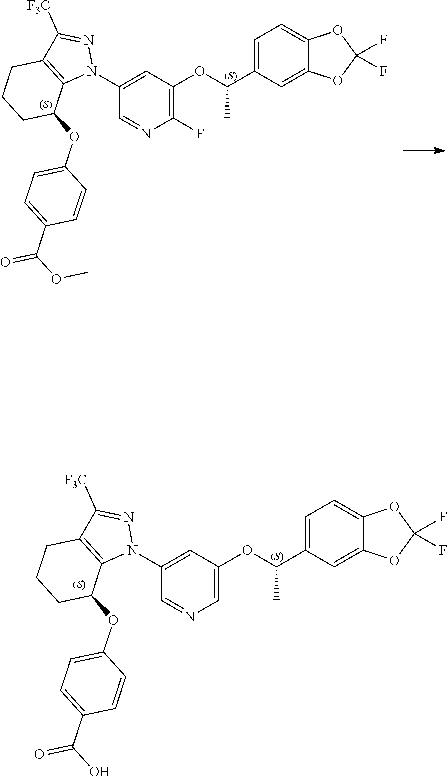

To a solution of methyl 4-[[(7S)-1-[5-[(1S)-1-(2,2-difluoro-1,3-benzodioxol-5-yl)ethoxy]-6-fluoro-3-pyridyl]-3-(trifluoromethyl)-4,5,6,7-tetrahydroindazol-7-yl]oxy]benzoate (40 mg, 0.0629 mmol) in THF (5 mL) and water (1 mL) was added lithium hydroxide monohydrate (13 mg, 0.315 mmol). The solution was stirred at rt for 8 h. To the reaction solution was added water (10 mL). The pH was adjusted to 5 with aqueous HCl (2 M) and then extracted with ethyl acetate (3×10 mL). The organic layers were combined, concentrated, and purified by prep-HPLC (NH₄HCO₃ modifier) to afford the title compound (25 mg, 63% yield) as a white solid.

LC-MS (Method 2): Retention time=1.79 min. MS (ESI) m/z 620.0 [M−H]⁺.

¹H NMR (400 MHZ, CH₃OH-d₄) δ 7.96 (d, J=8.8 Hz, 2H), 7.86 (t, J=2.0 Hz, 1H), 7.55 (dd, J=8.6, 2.2 Hz, 1H), 7.14 (t, J=4.9 Hz, 2H), 7.06 (dd, J=8.3, 1.6 Hz, 1H), 6.95 (d, J=8.9 Hz, 2H), 5.59 (d, J=3.4 Hz, 1H), 5.15 (q, J=6.4 Hz, 1H), 2.85 (d, J=16.2 Hz, 1H), 2.61 (ddd, J=16.2, 10.4, 5.9 Hz, 1H), 2.23 (d, J=11.4 Hz, 1H), 2.13-1.80 (m, 3H), 1.47 (d, J=6.3 Hz, 3H).

### Example 69

### 4-[[1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]amino]benzoic acid

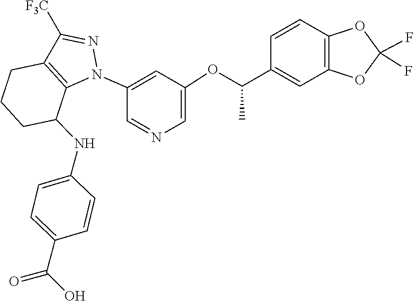 [SMILES: C[C@H](Oc1cncc(-n2nc(C(F)(F)F)c3c2C(Nc2ccc(C(=O)O)cc2)CCC3)c1)c1ccc2c(c1)OC(F)(F)O2]

### Step 1. Synthesis of 1-[5-[(1S)-1-(2,2-difluoro-1,3-benzodioxol-5-yl)ethoxy]-3-pyridyl]-3-(trifluoromethyl)-5,6-dihydro-4H-indazol-7-one

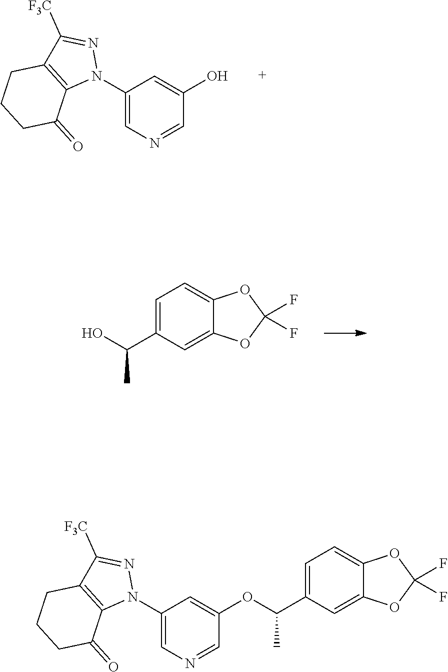 [SMILES: O=C1CCCc2c(C(F)(F)F)nn(-c3cncc(O)c3)c21 ; C[C@@H](O)c1ccc2c(c1)OC(F)(F)O2 ; C[C@H](Oc1cncc(-n2nc(C(F)(F)F)c3c2C(=O)CCC3)c1)c1ccc2c(c1)OC(F)(F)O2]

To a solution of 1-(5-hydroxy-3-pyridyl)-3-(trifluoromethyl)-5,6-dihydro-4H-indazol-7-one (Intermediate D-5) (2.90 g, 9.76 mmol) in anhydrous THF (20 mL) was added (1R)-1-(2,2-difluoro-1,3-benzodioxol-5-yl)ethanol (Intermediate C-1R) (2.37 g, 11.7 mmol) and triphenylphosphine (3.84 g, 14.6 mmol) at 0° C. under a nitrogen atmosphere with stirring. To this solution was added dropwise diethyl azodicarboxylate (2.3 mL, 14.6 mmol) over a period of 10 min and the reaction was monitored by LC-MS. After completion, the reaction solution was concentrated. The residue was purified by reversed-phase flash chromatography (C18) (0.1% NH₄HCO₃, 10-100% ACN) to give the crude product (3.2 g ee=75.88%). The crude product was separated by chiral-HPLC (IG 20×250 mm, 10 μm) to give the title compound (2.70 g, 57% yield) as a white solid. Chiral Method: Column Name: IG 4.6×100 mm 5 μm. Solvent: MeOH [0.2% NH₃ (7M in MeOH)]; Retention time=1.632 min; ee=99.88%.

LC-MS (Method 4): Retention time=2.29 min. MS (ESI) m/z 482.1 [M+H]⁺.

### Step 2. Synthesis of (7R)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-ol

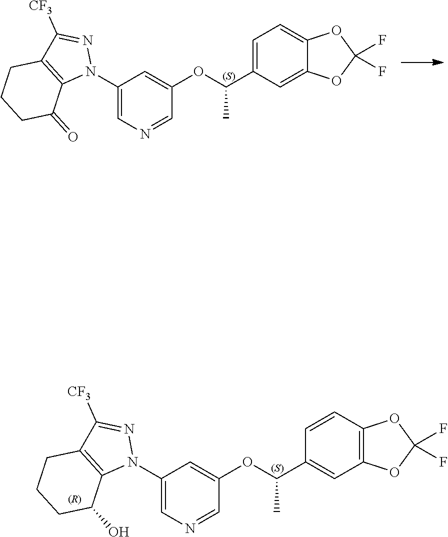

To a solution of 1-[5-[(1S)-1-(2,2-difluoro-1,3-benzodioxol-5-yl)ethoxy]-3-pyridyl]-3-(trifluoromethyl)-5,6-dihydro-4H-indazol-7-one (2.50 g, 5.19 mmol) in IPA (25 mL) was added triethylamine (1.5 mL, 10.8 mmol) and RuCl(p-cymene)[(R,R)-Ts-DPEN] (CAS No.: 192139-92-7) (33 mg, 0.0519 mmol) at 0° C., the solution was stirred at this temperature for 10 min under a nitrogen atmosphere. To this solution was added dropwise formic acid (1.0 mL, 26.1 mmol) slowly. Then the solution was stirred at 45° C. for 8 h. The reaction was monitored by LC-MS. After completion, the solution was diluted with water (30 mL) and extracted with EA (3×30 mL). The organic phases were combined and washed with brine and dried over Na₂SO₄. The organic solution was concentrated under a vacuum. The residue was purified by flash column chromatography (PE:EA=1:1) to give the title compound (2.50 g, 99.58% yield) as a white solid.

Chiral Method: Column Name: AD-H 4.6×100 mm 5 μm. Solvent: MeOH [0.2% NH₃ (7M in MeOH)]; Retention time=1.6 min; ee=100%.

LC-MS (Method 3): Retention time=1.87 min. MS (ESI) m/z 484.1 [M+H]⁺.

### Step 3. Synthesis of (7S)-7-chloro-1-[5-[(1S)-1-(2,2-difluoro-1,3-benzodioxol-5-yl)ethoxy]-3-pyridyl]-3-(trifluoromethyl)-4,5,6,7-tetrahydroindazole

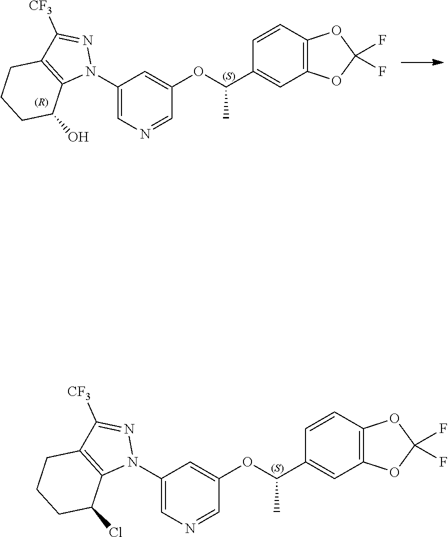

To a solution of (7R)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-ol (1.50 g, 3.10 mmol) and triethylamine (1.3 mL, 9.31 mmol) in DCM (50 mL) was added methanesulfonyl chloride (0.48 mL, 6.21 mmol) at 0° C. The solution was stirred at rt for 4 h. Then the solution was diluted with water (20 mL) and extracted with DCM (3×30 mL). The organic extracts were combined, washed with brine, and dried over Na₂SO₄. The organic solution was concentrated under vacuum. The residue was purified by flash column chromatography (PE:EA=5:1) to give the title compound (1.50 g, 96% yield) as a colorless oil.

LC-MS (Method 3): Retention time=1.98 min. MS (ESI) m/z 502.1 [M+H]⁺.

### Step 4. Synthesis of methyl 4-[[1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]amino]benzoate

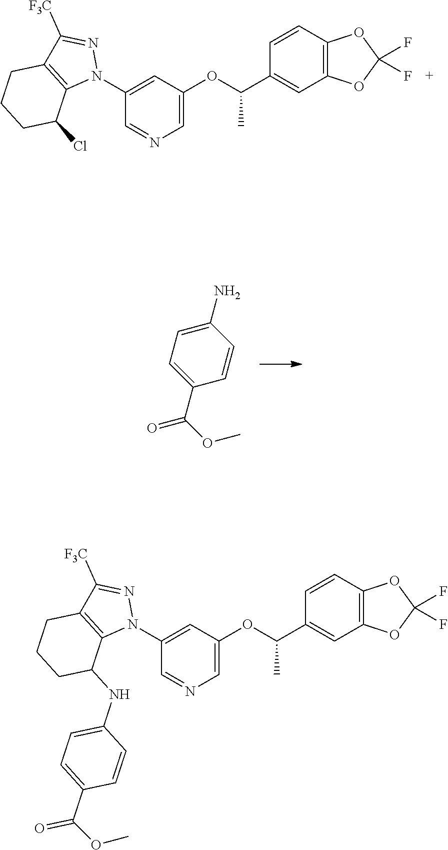

To a solution of (7S)-7-chloro-1-[5-[(1S)-1-(2,2-difluoro-1,3-benzodioxol-5-yl)ethoxy]-3-pyridyl]-3-(trifluoromethyl)-4,5,6,7-tetrahydroindazole (150 mg, 0.299 mmol) and methyl 4-aminobenzoate (54 mg, 0.359 mmol) in DMF (2 mL) was added potassium carbonate (207 mg, 1.49 mmol) and potassium iodide (25 mg, 0.149 mmol). The solution was stirred at 60° C. for 30 min. The reaction solution was concentrated and purified by reversed-phase flash chromatography (C18) (0.1% NH₄HCO₃ in water, 10-100% ACN) to give the title compound (80 mg, 43% yield) as a white solid.

LC-MS (Method 3): Retention time=1.95 min. MS (ESI) m/z 617.2 [M+H]⁺.

### Step 5. Synthesis of 4-[[1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]amino]benzoic acid

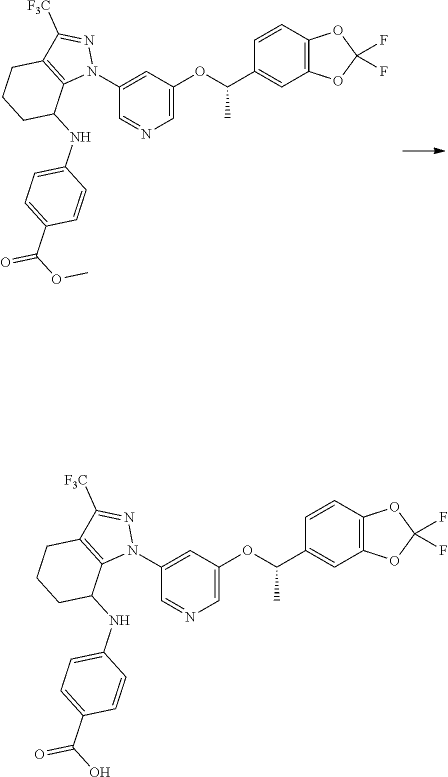 [SMILES: COC(=O)c1ccc(NC2CCCc3c(C(F)(F)F)nn(-c4cncc(O[C@@H](C)c5ccc6c(c5)OC(F)(F)O6)c4)c32)cc1 ; C[C@H](Oc1cncc(-n2nc(C(F)(F)F)c3c2C(Nc2ccc(C(=O)O)cc2)CCC3)c1)c1ccc2c(c1)OC(F)(F)O2]

To a solution of methyl 4-[[1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]amino]benzoate (50 mg, 0.0811 mmol) in methanol (1.5 mL) and THF (1.5 mL) was added aqueous sodium hydroxide (1.2 mL, 2.4 mmol, 2 M) at 0° C. Then the reaction solution was stirred at rt for 5 h. The reaction was monitored by LC-MS. After completion of the reaction, the solution was adjusted to pH 5-6 by adding citric acid. Then the solution was diluted with water (10 mL) and extracted with ethyl acetate (3×20 mL). The organic extracts were combined and washed with brine and dried over Na₂SO₄. The organic solution was concentrated under vacuum. The residue was purified by reversed-phase flash chromatography (C18) (0.1% NH₄HCO₃ in water, 10-100% ACN) to give the title compound (29 mg, 60% yield) as a white solid.

LC-MS (Method 3): Retention time=1.82 min. MS (ESI) m/z 603.1 [M+H]⁺.

¹H NMR (500 MHZ, CH₃OH-d₄) δ 8.36 (dd, J=28.5, 1.9 Hz, 1H), 8.13 (dd, J=25.1, 2.5 Hz, 1H), 7.81 (dd, J=23.9, 8.7 Hz, 2H), 7.67 (dt, J=22.0, 2.3 Hz, 1H), 7.17-7.03 (m, 2H), 6.98 (d, J=8.3 Hz, 1H), 6.63 (dd, J=17.9, 8.8 Hz, 2H), 5.15 (dq, J=43, 6 Hz, 1H), 4.92 (dd, J=45, 3.5 Hz, 1H), 2.82 (d, J=17.1 Hz, 1H), 2.62 (m, 1H), 2.07 (m, 1H), 1.89 (m, 3H), 1.44 (dd, J=22.1, 6.3 Hz, 3H).

### Example 70

### 4-((1-(3-((S)-1-(2,2-difluorobenzo[d][1,3]dioxol-5-yl)ethoxy)phenyl)-3-(trifluoromethyl)-4,5,6,7-tetrahydro-1H-indazol-7-yl)thio)benzoic acid

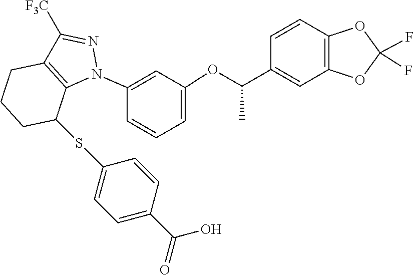 [SMILES: C[C@H](Oc1cccc(-n2nc(C(F)(F)F)c3c2C(Sc2ccc(C(=O)O)cc2)CCC3)c1)c1ccc2c(c1)OC(F)(F)O2]

### Step 1. Synthesis of 4-[[1-[3-[(1S)-1-(2,2-difluoro-1,3-benzodioxol-5-yl)ethoxy]phenyl]-3-(trifluoromethyl)-4,5,6,7-tetrahydroindazol-7-yl]sulfanyl]benzoic acid

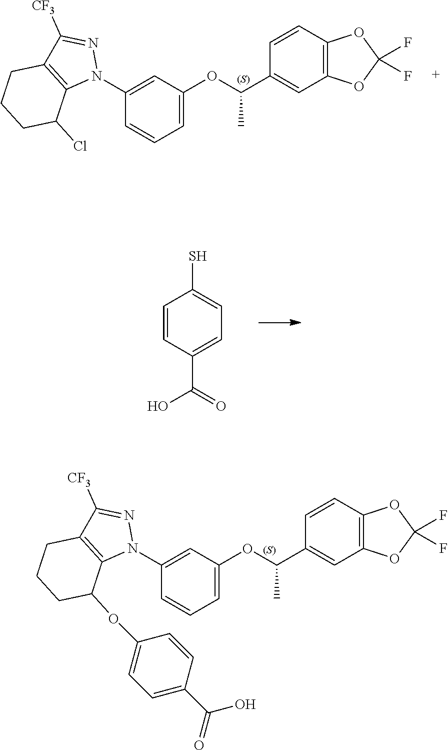

To a solution of 7-chloro-1-[3-[(1S)-1-(2,2-difluoro-1,3-benzodioxol-5-yl)ethoxy]phenyl]-3-(trifluoromethyl)-4,5,6,7-tetrahydroindazole (from Intermediate of Step 3b, Examples 37 and 38) (150 mg, 0.299 mmol) and 4-mercaptobenzoic acid (55 mg, 0.359 mmol) in DMF (2 mL) was added potassium carbonate (207 mg, 1.50 mmol) and potassium iodide (25 mg, 0.150 mmol). The solution was stirred at 60° C. for 30 min. The reaction solution was concentrated, and the residue was purified by reversed-phase flash chromatography (C18) (0.1% NH₄HCO₃ in water, 10-100% ACN) to give the title compound (150 mg, 81% yield) as a white solid.

LC-MS (Method 4): Retention time=1.96 min. MS (ESI) m/z 619.1 [M+H]⁺.

¹H NMR (500 MHZ, CH₃OH-d₄) δ 7.83-7.75 (m, 2H), 7.33 (td, J=8.2, 1.7 Hz, 1H), 7.22 (dd, J=8.1, 1.5 Hz, 1H), 7.18-6.97 (m, 7H), 5.43 (dq, J=22.0, 6.4 Hz, 1H), 4.51 (m 1H), 2.77 (d, J=15.8 Hz, 1H), 2.60-2.50 (m, 1H), 2.13-2.00 (m, 3H), 1.96-1.87 (m, 1H), 1.57 (dd, J=8.4, 6.4 Hz, 3H).

### Example 71

### 4-[[1-[3-[(1S)-1-(2,2-difluoro-1,3-benzodioxol-5-yl)ethoxy]phenyl]-3-(trifluoromethyl)-4,5,6,7-tetrahydroindazol-7-yl]sulfonyl]benzoic acid

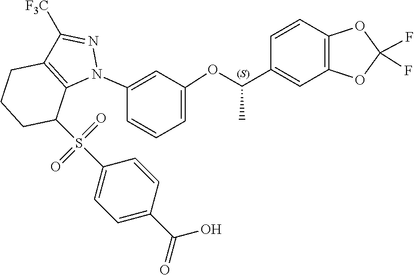

### Step 1. Synthesis of 4-[1-[3-[[(1S)-1-(2,2-difluoro-1,3-benzodioxol-5-yl)ethoxy]phenyl]-3-(trifluoromethyl)-4,5,6,7-tetrahydroindazol-7-yl]sulfonyl]benzoic acid

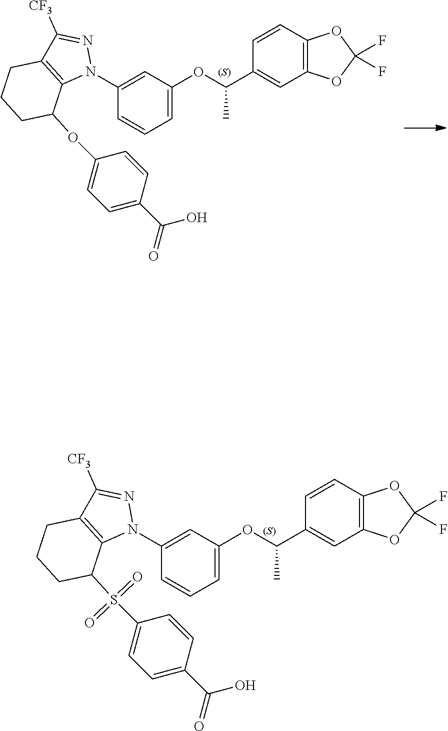

To a solution of 4-[[1-[3-[(1S)-1-(2,2-difluoro-1,3-benzodioxol-5-yl)ethoxy]phenyl]-3-(trifluoromethyl)-4,5,6,7-tetrahydroindazol-7-yl]sulfanyl]benzoic acid (Example 70) (60 mg, 0.0970 mmol) in DCM (3 mL) was added 3-chloroperoxybenzoic acid (33 mg, 0.194 mmol). The solution was stirred at rt for 4 h. The reaction was monitored by LC-MS. After completion of the reaction, the reaction solution was concentrated and the residue was purified by reverse-phase flash chromatography (C18) (0.1% NH₄HCO₃ in water, 10-100% ACN) to give the title compound (26 mg, 41.0% yield) as a white solid.

LC-MS (Method 4): Retention time=1.87 min. MS (ESI) m/z 651.1 [M+H]⁺.

¹H NMR (500 MHZ, CH₃OH-d₄) δ 7.88 (d, J=8.4 Hz, 2H), 7.38 (d, J=8.4 Hz, 1H), 7.32-7.10 (m, 5H), 6.95-6.70 (m, 3H), 5.45 (dq, J=37.2, 6.3 Hz, 1H), 5.09 (d, J=3.6 Hz, 0.46H), 4.58 (d, J=3.8 Hz, 0.55H), 2.82 (dd, J=16.7, 6.2 Hz, 1H), 2.71-2.55 (m, 2H), 2.38-2.24 (m, 1H), 2.16-2.01 (m, 1H), 1.98-1.87 (m, 1H), 1.62 (dd, J=12.1, 6.4 Hz, 3H).

### Examples 72 and 73

### 4-[[(7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]sulfanyl]benzoic acid

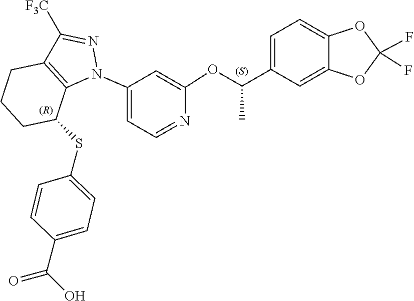

### 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]sulfanyl]benzoic acid

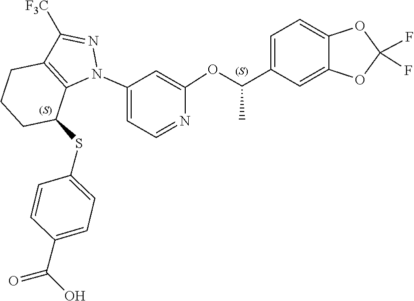

### Step 1. Synthesis of 4-(((S)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)thio)benzoic acid and 4-(((R)-1-(2-((S)-1-(2,2-difluorobenzo[d][1,3]dioxol-5-yl)ethoxy)pyridine-4-yl)-3-(trifluoromethyl)-4,5,6,7-tetrahydro-1H-indazol-7-yl)thio)benzoic acid

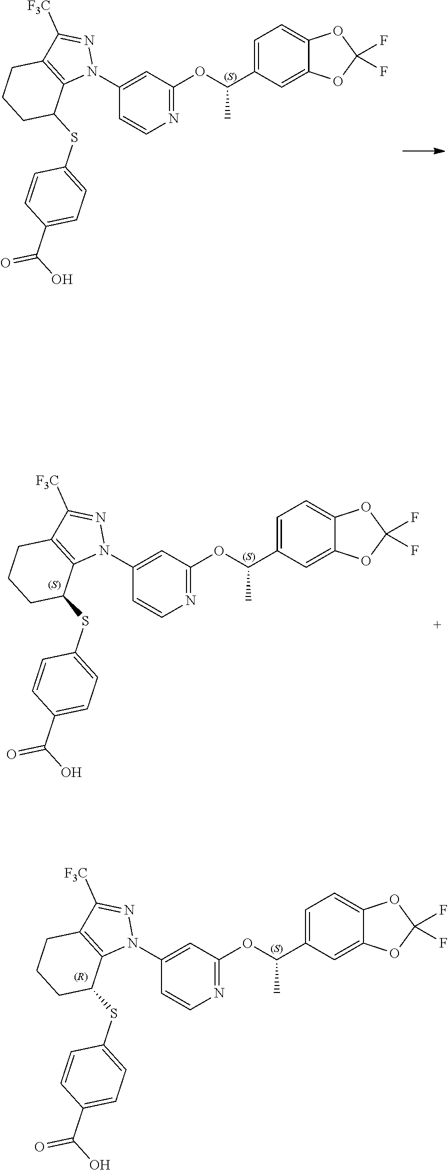

Example 71 was separated by SFC to the title compounds with the first fraction designated as P1 (27.1 mg, 12% yield; a white solid) and the second fraction as P2 (26.9 mg, 13%; a white solid).

P1: LC-MS (Method 2): Retention time=1.88 min. MS (ESI) m/z=618.0 (M−H)⁺.

¹H NMR (400 MHZ, CH₃OH-d₄) δ 8.19-8.07 (m, 1H), 7.89 (d, J=8.1 Hz, 2H), 7.34 (d, J=8.0 Hz, 2H), 7.30-7.17 (m, 3H), 7.14 (dd, J=8.3, 1.7 Hz, 1H), 7.06 (d, J=8.3 Hz, 1H), 6.15 (q, J=6.5 Hz, 1H), 5.03 (s, 1H), 2.80 (d, J=16.3 Hz, 1H), 2.60 (dd, J=16.4, 10.2 Hz, 1H), 2.15 (s, 3H), 1.94 (s, 1H), 1.59 (d, J=6.5 Hz, 3H).

P2: LC-MS (Method 2): Retention time=1.87 min. MS (ESI) m/z=618.0 (M−H)⁺.

¹H NMR (400 MHZ, CH₃OH-d₄) δ 8.12 (d, J=5.6 Hz, 1H), 7.89 (d, J=8.1 Hz, 2H), 7.31 (d, J=8.0 Hz, 2H), 7.29-7.19 (m, 3H), 7.20-7.04 (m, 2H), 6.13 (q, J=6.5 Hz, 1H), 4.92 (d, J=3.2 Hz, 1H), 2.79 (d, J=16.1 Hz, 1H), 2.68-2.50 (m, 1H), 2.26-2.02 (m, 3H), 1.95 (d, J=12.4 Hz, 1H), 1.56 (d, J=6.5 Hz, 3H).

### Example 74 (P1 and P2)

### 3-[[(7S)-1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid

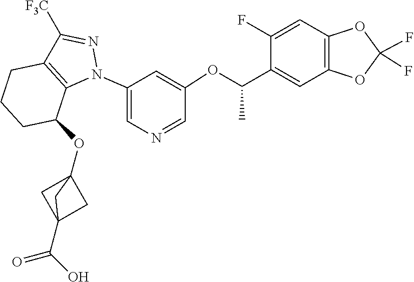 [SMILES: C[C@H](Oc1cncc(-n2nc(C(F)(F)F)c3c2[C@@H](OC24CC(C(=O)O)(C2)C4)CCC3)c1)c1cc2c(cc1F)OC(F)(F)O2]

### 3-[[(7R)-1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid

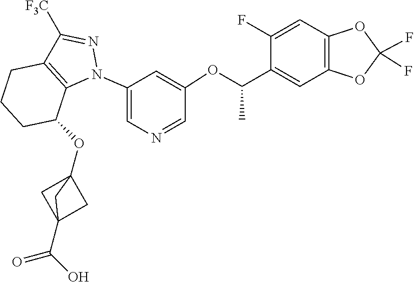 [SMILES: C[C@H](Oc1cncc(-n2nc(C(F)(F)F)c3c2[C@H](OC24CC(C(=O)O)(C2)C4)CCC3)c1)c1cc2c(cc1F)OC(F)(F)O2]

### Step 1. Synthesis of 7-chloro-1-[5-[(1S)-1-(2,2,6-trifluoro-1,3-benzodioxol-5-yl)ethoxy]-3-pyridyl]-3-(trifluoromethyl)-4,5,6,7-tetrahydroindazole

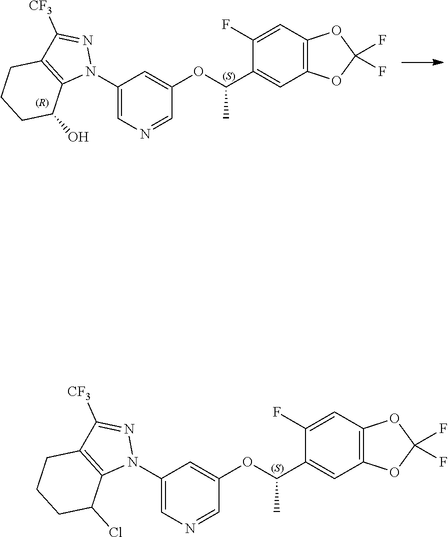

To a solution of (7R)-1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-ol (synthesized similarly to Example 36, step 2) (300 mg, 0.598 mmol) and triethylamine (0.25 mL, 1.80 mmol) in DCM (15 mL) was added methanesulfonyl chloride (0.061 mL, 0.778 mmol) at 0° C. The reaction was stirred at rt for 4 h. Then the mixture was diluted with water (30 mL) and extracted with DCM (30 mL×3). The combined organic phases were washed with brine, dried over Na₂SO₄, and concentrated under vacuum. The residue was purified by flash column chromatography (PE:EA=5:1) to give 7-chloro-1-[5-[(1S)-1-(2,2,6-trifluoro-1,3-benzodioxol-5-yl)ethoxy]-3-pyridyl]-3-(trifluoromethyl)-4,5,6,7-tetrahydroindazole (300 mg, 0.577 mmol, 96.4% yield) as a colorless oil.

LCMS (Method 1): Retention time=2.36 min. MS (ESI) m/z 520.0 [M+H]⁺.

### Step 2. Synthesis of methyl 3-[[1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylate

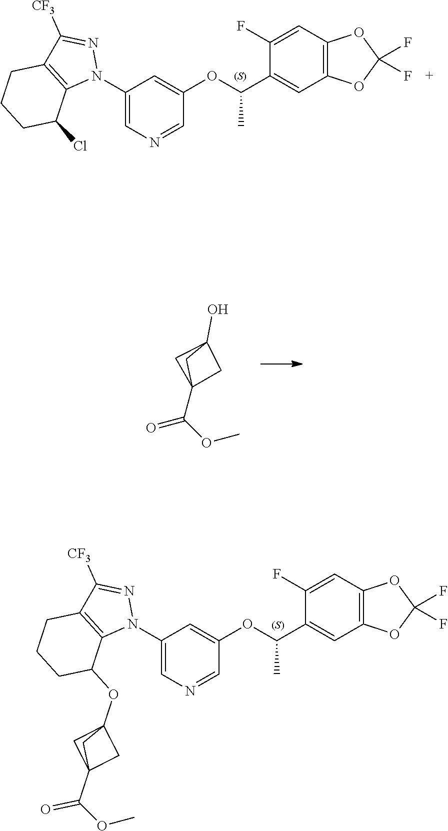

To a solution of methyl 3-hydroxybicyclo[1.1.1]pentane-1-carboxylate (66 mg, 0.462 mmol) in dry 2,6-di-tert-butylpyridine (177 mg, 0.923 mmol) was added silver trifluoromethanesulfonate (198 mg, 0.769 mmol) at 0° C., followed by (7S)-7-chloro-1-[5-[(1S)-1-(2,2,6-trifluoro-1,3-benzodioxol-5-yl)ethoxy]-3-pyridyl]-3-(trifluoromethyl)-4,5,6,7-tetrahydroindazole (160 mg, 0.308 mmol) dropwise. The reaction was stirred at 0° C. for 1.5 h. Et3N were added to maintain basic conditions. The mixture was filtered through a Celite pad. The filtrate was concentrated and purified by reversed-phase flash chromatography (C18) (0.1% NH₄HCO₃ in water, 10-100% acetonitrile) to give methyl 3-[[1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylate (120 mg, 0.192 mmol, 62.3% yield) as a white solid.

LCMS (Method 1): Retention time=2.38 min. MS (ESI) m/z 626.0 [M+H]⁺.

### Step 3. Synthesis of 3-[[1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid

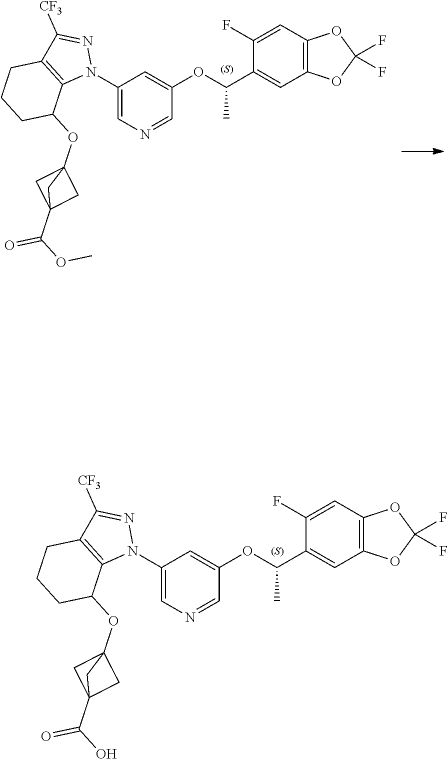

To a solution of methyl 3-[[1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylate (120 mg, 0.192 mmol) in methanol (1.5 mL) and THF (1.5 mL) was added sodium hydroxide (2.9 mL, 5.76 mmol) at 0° C. The reaction mixture was stirred at rt for 5 h. The reaction was monitored by LCMS. The reaction mixture was adjusted to pH 5-6 by addition of citric acid. Then the mixture was diluted with water (10 mL) and extracted with ethyl acetate (20 mL×3). The combined organic phase was washed with brine, dried over Na₂SO₄, and concentrated under vacuum. The residue was purified by reversed-phase flash chromatography (C18) (0.1% NH₄HCO₃ in water, 10-100% acetonitrile) to give 3-[[1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid (100 mg, 0.164 mmol, 85.2% yield) as a white solid.

LCMS (Method 1): Retention time=2.22 min. MS (ESI) m/z 612.0 [M+H]⁺.

### Step 4. Synthesis of 3-[[(7S)-1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid (P1) and 3-[[(7R)-1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid (P2)

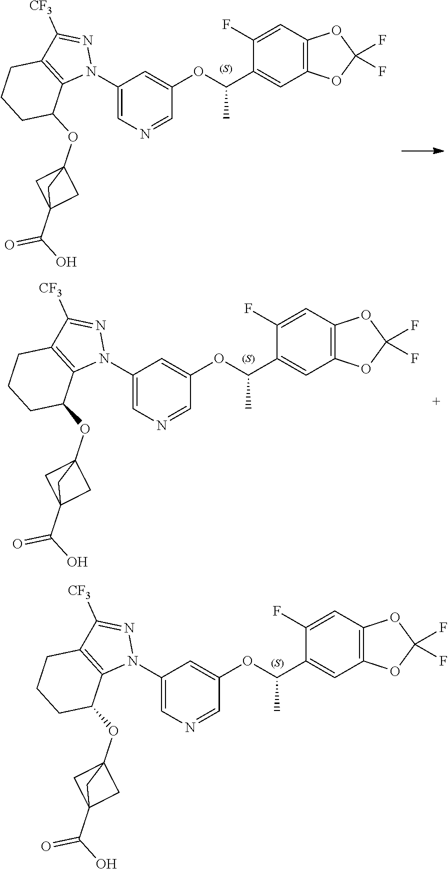

The crude of 3-[[1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid (100 mg, 0.164 mmol) was separated by chiral SFC to give 3-[[(7S)-1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid (27 mg, 0.0429 mmol, 26.2% yield) (P1) (ee: 100%) as a white solid and 3-[[(7R)-1-[5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid (26 mg, 0.0413 mmol, 25.2% yield) (P2) (ee: 95.9%) as a white solid.

### Chiral Prep SFC Conditions:

Instrument: SFC-150 (Waters)
Column: IG 20*250 mm, 10 μm
Column temperature: 35° C.
Mobile phase: CO₂/EtOH: n-Hexane=1:1 (0.2% Methanol Ammonia (7M))=80/20
Flow rate: 100 g/min
Back pressure: 100 bar
Detection wavelength: 214 nm
Cycle time: 6 min
Sample solution: 100 mg dissolved in 30 ml Methanol
Injection volume: 0.6 ml

P1: LCMS (Method 2): Retention time=1.72 min. MS (ESI) m/z 612.0 [M+H]⁺.

Chiral Method: Column Name: IG 4.6*100 mm 3.5 μm Solvent: EtOH: n-Hexane=1:1 [0.2% NH₃ (7M in MeOH)]; Find at 2.236 min; ee: 100%

¹H NMR (400 MHZ, MeOH-d4) δ 8.40 (dd, J=11.0, 1.9 Hz, 2H), 7.62 (s, 1H), 7.42 (d, J=5.6 Hz, 1H), 7.18 (d, J=9.0 Hz, 1H), 5.92 (q, J=6.2 Hz, 1H), 4.82 (s, 1H), 2.77 (d, J=16.1 Hz, 1H), 2.63-2.50 (m, 1H), 2.12-2.02 (m, 4H), 1.99-1.79 (m, 6H), 1.73 (d, J=6.3 Hz, 3H).

P2: LCMS (Method 2): Retention time=1.72 min. MS (ESI) m/z 612.0 [M+H]⁺.

Chiral Method: Column Name: IG 4.6*100 mm 3.5 μm Solvent: EtOH: n-Hexane=1:1 [0.2% NH₃ (7M in MeOH)]; Find at 2.931 min; ee: 95.88%

¹H NMR (400 MHZ, MeOH-d4) δ 8.40 (dd, J=15.4, 2.0 Hz, 2H), 7.63 (s, 1H), 7.44 (d, J=5.6 Hz, 1H), 7.19 (d, J=9.1 Hz, 1H), 5.92 (q, J=6.3 Hz, 1H), 4.78 (s, 1H), 2.77 (d, J=16.6 Hz, 1H), 2.63-2.49 (m, 1H), 2.12-2.01 (m, 4H), 1.99-1.79 (m, 6H), 1.73 (d, J=6.3 Hz, 3H).

Example 75 to Example 84 were synthesized according to the protocols described in the synthesis of Example 74:

### Example 75 (P1 and P2)

### 3-[[(7S)-1-[6-fluoro-5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid

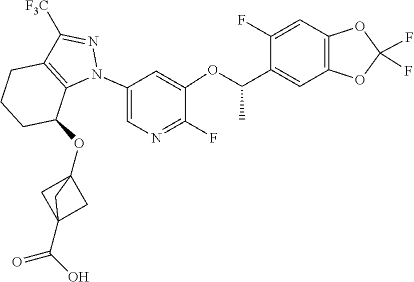 [SMILES: C[C@H](Oc1cc(-n2nc(C(F)(F)F)c3c2[C@@H](OC24CC(C(=O)O)(C2)C4)CCC3)cnc1F)c1cc2c(cc1F)OC(F)(F)O2]

### 3-[[(7R)-1-[6-fluoro-5-[(1S)-1-(2,2,6-trifluoro-1,3-benzodioxol-5-yl)ethoxy]-3-pyridyl]-3-(trifluoromethyl)-4,5,6,7-tetrahydroindazol-7-yl]oxy]bicyclo[1.1.1]pentane-1-carboxylic acid

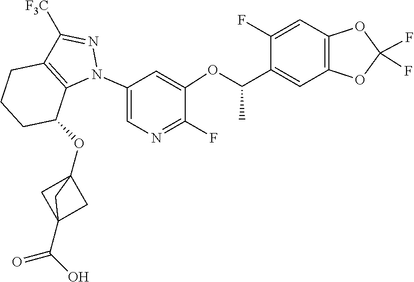 [SMILES: C[C@H](Oc1cc(-n2nc(C(F)(F)F)c3c2[C@H](OC24CC(C(=O)O)(C2)C4)CCC3)cnc1F)c1cc2c(cc1F)OC(F)(F)O2]

P1: LCMS (Method 2): Retention time=1.75 min. MS (ESI) m/z 630.0 [M+H]⁺.

¹H NMR (400 MHZ, MeOH-d4) δ 7.96 (s, 1H), 7.58 (dd, J=8.6, 2.1 Hz, 1H), 7.37 (d, J=5.7 Hz, 1H), 7.12 (d, J=9.0 Hz, 1H), 5.85 (q, J=6.3 Hz, 1H), 4.74 (d, J=3.7 Hz, 1H), 2.70 (d, J=16.5 Hz, 1H), 2.48 (dd, J=15.9, 8.9 Hz, 1H), 2.06-1.89 (m, 4H), 1.83 (d, J=9.3 Hz, 6H), 1.68 (d, J=6.3 Hz, 3H).

P2: LCMS (Method 2): Retention time=1.75 min. MS (ESI) m/z 630.0 [M+H]⁺.

¹H NMR (400 MHZ, MeOH-d4) δ 7.97 (t, J=1.9 Hz, 1H), 7.62 (dd, J=8.6, 2.1 Hz, 1H), 7.39 (d, J=5.7 Hz, 1H), 7.13 (d, J=9.1 Hz, 1H), 5.84 (q, J=6.3 Hz, 1H), 4.65 (t, J=3.5 Hz, 1H), 2.70 (d, J=16.2 Hz, 1H), 2.58-2.42 (m, 1H), 2.05-1.93 (m, 4H), 1.91-1.75 (m, 6H), 1.68 (d, J=6.3 Hz, 3H).

### Example 76 (P1 and P2)

### 3-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]bicyclo[1.1.1]pentane-1-carboxylic acid

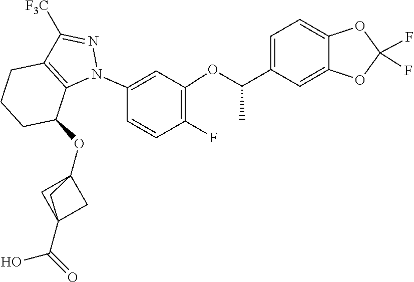 [SMILES: C[C@H](Oc1cc(-n2nc(C(F)(F)F)c3c2[C@@H](OC24CC(C(=O)O)(C2)C4)CCC3)ccc1F)c1ccc2c(c1)OC(F)(F)O2]

### 3-[[(7R)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]bicyclo[1.1.1]pentane-1-carboxylic acid

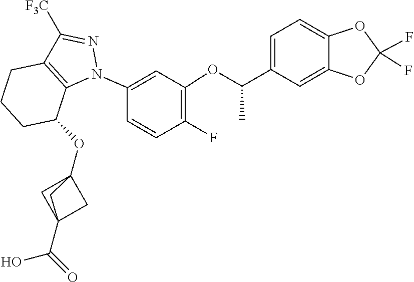 [SMILES: C[C@H](Oc1cc(-n2nc(C(F)(F)F)c3c2[C@H](OC24CC(C(=O)O)(C2)C4)CCC3)ccc1F)c1ccc2c(c1)OC(F)(F)O2]

P1: LCMS (Method 2): Retention time=1.74 min. MS (ESI) m/z 611.1 [M+H]⁺.

¹H NMR (400 MHZ, MeOH-d4) δ 7.33 (d, J=1.6 Hz, 1H), 7.26 (ddd, J=8.3, 7.2, 5.1 Hz, 2H), 7.19-7.13 (m, 2H), 7.07 (ddd, J=8.6, 3.8, 2.5 Hz, 1H), 5.53 (q, J=6.3 Hz, 1H), 4.44 (t, J=3.3 Hz, 1H), 2.72 (d, J=16.1 Hz, 1H), 2.48 (ddd, J=16.4, 9.9, 6.5 Hz, 1H), 2.05-1.94 (m, 1H), 1.90-1.69 (m, 9H), 1.64 (d, J=6.4 Hz, 3H).

P2: LCMS (Method 2): Retention time=1.74 min. MS (ESI) m/z 611.1 [M+H]⁺.

¹H NMR (400 MHZ, MeOH-d4) δ 7.34-7.15 (m, 4H), 7.15-7.01 (m, 2H), 5.56 (q, J=6.3 Hz, 1H), 4.70 (t, J=3.3 Hz, 1H), 2.72 (d, J=16.3 Hz, 1H), 2.56-2.42 (m, 1H), 2.06-1.96 (m, 1H), 1.81 (m, 9H), 1.64 (d, J=6.4 Hz, 3H).

### Example 77 (P1 and P2)

### 3-[[(7S)-1-[6-[(1S)-1-(2,2-difluoro-1,3-benzodioxol-5-yl)ethoxy]-2-pyridyl]-3-(trifluoromethyl)-4,5,6,7-tetrahydroindazol-7-yl]oxy]bicyclo[1.1.1]pentane-1-carboxylic acid

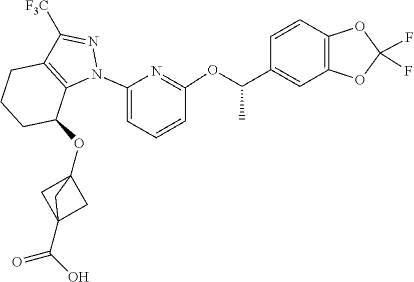 [SMILES: C[C@H](Oc1cccc(-n2nc(C(F)(F)F)c3c2[C@@H](OC24CC(C(=O)O)(C2)C4)CCC3)n1)c1ccc2c(c1)OC(F)(F)O2]

and

### 3-[[(7R)-1-[6-[(1S)-1-(2,2-difluoro-1,3-benzodioxol-5-yl)ethoxy]-2-pyridyl]-3-(trifluoromethyl)-4,5,6,7-tetrahydroindazol-7-yl]oxy]bicyclo[1.1.1]pentane-1-carboxylic acid

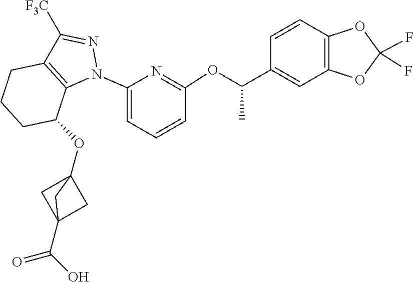 [SMILES: C[C@H](Oc1cccc(-n2nc(C(F)(F)F)c3c2[C@H](OC24CC(C(=O)O)(C2)C4)CCC3)n1)c1ccc2c(c1)OC(F)(F)O2]

P1: LC-Mass (Method 2): Retention time=1.76 min. MS (ESI, neg.) m/z 592.0 [M−H]⁺. Chiral Method: Column Name: OD 4.6×100 mm 3 μm; Solvent: MeOH [0.2% NH₃ (7M in MeOH)]; Find at 2.697 min; ee: 100%

¹H NMR (400 MHZ, MeOH-d4-d4) δ 7.95-7.85 (m, 1H), 7.40 (d, J=1.5 Hz, 1H), 7.33 (dd, J=8.3, 1.5 Hz, 1H), 7.21 (d, J=8.3 Hz, 1H), 7.16 (d, J=7.5 Hz, 1H), 6.99 (d, J=8.2 Hz, 1H), 6.04 (q, J=6.5 Hz, 1H), 4.81 (s, 1H), 2.74 (d, J=16.1 Hz, 1H), 2.62-2.46 (m, 1H), 1.96-1.77 (m, 4H), 1.67-1.57 (m, 6H), 1.52 (m, 3H).

P2: LC-Mass (Method 2): Retention time=1.76 min. MS (ESI, neg.) m/z 592.0 [M−H]⁺. Chiral Method: Column Name: OD 4.6×100 mm; 3 μm. Solvent: MeOH [0.2% NH₃ (7M in MeOH)]; Find at 3.481 min; ee: 98.78%

¹H NMR (400 MHZ, MeOH-d4) δ 7.86 (t, J=7.9 Hz, 1H), 7.28 (d, J=1.5 Hz, 1H), 7.23-7.13 (m, 2H), 7.07 (d, J=8.3 Hz, 1H), 6.93 (d, J=8.1 Hz, 1H), 6.13 (q, J=6.6 Hz, 1H), 5.47 (t, J=3.6 Hz, 1H), 2.74 (d, J=16.4 Hz, 1H), 2.63-2.49 (m, 1H), 2.15-2.07 (m, 1H), 2.04-1.80 (m, 10H), 1.69 (d, J=6.6 Hz, 3H).

### Example 78 (P1 and P2)

### 2-[3-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-1-bicyclo[1.1.1]pentanyl]acetic acid

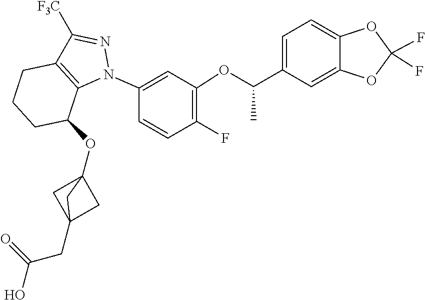 [SMILES: C[C@H](Oc1cc(-n2nc(C(F)(F)F)c3c2[C@@H](OC24CC(CC(=O)O)(C2)C4)CCC3)ccc1F)c1ccc2c(c1)OC(F)(F)O2]

### 2-[3-[[(7R)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-1-bicyclo[1.1.1]pentanyl]acetic acid

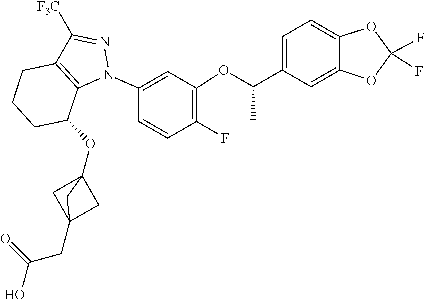 [SMILES: C[C@H](Oc1cc(-n2nc(C(F)(F)F)c3c2[C@H](OC24CC(CC(=O)O)(C2)C4)CCC3)ccc1F)c1ccc2c(c1)OC(F)(F)O2]

P1: LCMS (Method 4): Retention time=1.82 min. MS (ESI) m/z 625.0 [M+H]⁺.

¹H NMR (400 MHZ, MeOH-d4) δ:7.34 (d, J=1.6 Hz, 1H), 7.30-7.22 (m, 2H), 7.21-7.14 (m, 2H), 7.08 (ddd, J=8.6, 3.8, 2.5 Hz, 1H), 5.54 (q, J=6.3 Hz, 1H), 4.39 (t, J=3.3 Hz, 1H), 2.74 (d, J=15.9 Hz, 1H), 2.55-2.47 (m, 1H), 2.45 (s, 2H), 2.03 (dd, J=9.3, 5.9 Hz, 1H), 1.83 (dd, J=14.2, 10.8 Hz, 2H), 1.71-1.63 (m, 4H), 1.61 (dd, J=9.3, 1.0 Hz, 3H), 1.54-1.45 (m, 3H).

P2: LCMS (Method 4): Retention time=1.82 min. MS (ESI) m/z 625.0 [M+H]⁺.

¹H NMR (400 MHZ, MeOH-d4) δ:7.33-7.21 (m, 3H), 7.20-7.13 (m, 2H), 7.10 (ddd, J=8.6, 3.9, 2.5 Hz, 1H), 5.57 (q, J=6.4 Hz, 1H), 4.68 (t, J=3.3 Hz, 1H), 2.73 (d, J=16.6 Hz, 1H), 2.56-2.48 (m, 1H), 2.47 (s, 2H), 2.04 (d, J=11.3 Hz, 1H), 1.93-1.68 (m, 3H), 1.68-1.59 (m, 6H), 1.58-1.51 (m, 3H).

### Example 79 (P1 and P2)

### 4-[[(7S)-1-[5-[(1S)-1-(2,2-difluoro-1,3-benzodioxol-5-yl)ethoxy]-6-fluoro-3-pyridyl]-3-(trifluoromethyl)-4,5,6,7-tetrahydroindazol-7-yl]oxy]cyclohexanecarboxylic acid

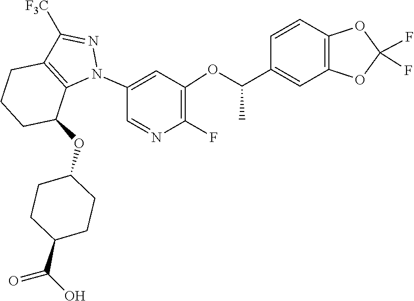 [SMILES: C[C@H](Oc1cc(-n2nc(C(F)(F)F)c3c2[C@@H](O[C@H]2CC[C@H](C(=O)O)CC2)CCC3)cnc1F)c1ccc2c(c1)OC(F)(F)O2]

### 4-[[(7R)-1-[5-[(1S)-1-(2,2-difluoro-1,3-benzodioxol-5-yl)ethoxy]-6-fluoro-3-pyridyl]-3-(trifluoromethyl)-4,5,6,7-tetrahydroindazol-7-yl]oxy]cyclohexanecarboxylic acid

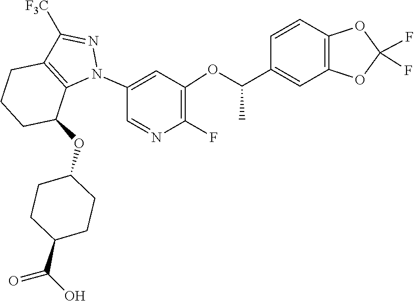 [SMILES: C[C@H](Oc1cc(-n2nc(C(F)(F)F)c3c2[C@@H](O[C@H]2CC[C@H](C(=O)O)CC2)CCC3)cnc1F)c1ccc2c(c1)OC(F)(F)O2]

P1: LCMS (Method 1): LC retention time=1.80 min, MS (ESI): m/z 628.0 [M+H]⁺.

¹H NMR (400 MHZ, MeOH-d4) δ 7.97 (s, 1H), 7.62 (dd, J=8.6, 2.0 Hz, 1H), 7.33 (s, 1H), 7.30-7.24 (m, 1H), 7.19 (d, J=8.3 Hz, 1H), 5.66 (q, J=6.3 Hz, 1H), 4.73 (d, J=3.8 Hz, 1H), 3.22-3.10 (m, 1H), 2.72 (d, J=16.6 Hz, 1H), 2.60-2.44 (m, 1H), 2.00 (m, 2H), 1.91-1.74 (m, 5H), 1.70 (t, J=8.8 Hz, 4H), 1.41-1.10 (m, 3H), 0.93 (ddd, J=16.5, 13.0, 3.7 Hz, 1H), 0.41 (m, 1H).

P2: LCMS (Method 1): LC retention time=1.80 min, MS (ESI): m/z 628.0 [M+H]⁺.

¹H NMR (400 MHZ, MeOH-d4) δ 7.92 (t, J=1.8 Hz, 1H), 7.65 (dd, J=8.7, 2.1 Hz, 1H), 7.39 (d, J=1.4 Hz, 1H), 7.31 (dd, J=8.3, 1.5 Hz, 1H), 7.24 (d, J=8.3 Hz, 1H), 5.61 (q, J=6.3 Hz, 1H), 4.27 (t, J=3.3 Hz, 1H), 2.92 (ddd, J=14.5, 10.3, 4.0 Hz, 1H), 2.73 (d, J=16.3 Hz, 1H), 2.52 (ddd, J=16.2, 9.9, 6.0 Hz, 1H), 2.08-1.95 (m, 2H), 1.93-1.77 (m, 3H), 1.72 (dd, J=13.2, 4.9 Hz, 6H), 1.39-1.00 (m, 4H), 0.53 (ddd, J=23.2, 12.8, 3.7 Hz, 1H).

### Example 80 (P1 and P2)

### 4-[[(7S)-1-[2-[(1S)-1-(2,2-difluoro-1,3-benzodioxol-5-yl)ethoxy]-6-fluoro-4-pyridyl]-3-(trifluoromethyl)-4,5,6,7-tetrahydroindazol-7-yl]oxy]cyclohexanecarboxylic acid (P1)

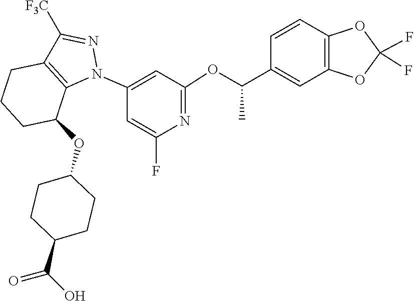 [SMILES: C[C@H](Oc1cc(-n2nc(C(F)(F)F)c3c2[C@@H](O[C@H]2CC[C@H](C(=O)O)CC2)CCC3)cc(F)n1)c1ccc2c(c1)OC(F)(F)O2]

### 4-[[(7R)-1-[2-[(1S)-1-(2,2-difluoro-1,3-benzodioxol-5-yl)ethoxy]-6-fluoro-4-pyridyl]-3-(trifluoromethyl)-4,5,6,7-tetrahydroindazol-7-yl]oxy]cyclohexanecarboxylic acid (P2)

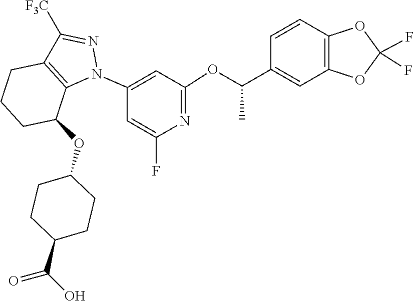 [SMILES: C[C@H](Oc1cc(-n2nc(C(F)(F)F)c3c2[C@@H](O[C@H]2CC[C@H](C(=O)O)CC2)CCC3)cc(F)n1)c1ccc2c(c1)OC(F)(F)O2]

P1: LCMS (Method 2): Retention time=1.96 min. MS (ESI) m/z 628.1 [M+H]⁺.

¹H NMR (400 MHZ, MeOH-d4) δ 7.32 (s, 1H), 7.27 (d, J=8.3 Hz, 1H), 7.21 (s, 1H), 7.17 (d, J=8.3 Hz, 1H), 6.92 (s, 1H), 6.14 (q, J=6.5 Hz, 1H), 4.86-4.85 (m, 1H), 3.51 (m, 1H), 2.74 (d, J=16.6 Hz, 1H), 2.60-2.47 (m, 1H), 2.28-2.07 (m, 2H), 2.01-1.77 (m, 6H), 1.66 (m, 4H), 1.39 (m, 3H), 1.13 (m, 1H).

P2: LCMS (Method 2): Retention time=1.96 min. MS (ESI) m/z 628.1 [M+H]⁺.

¹H NMR (400 MHZ, MeOH-d4) δ 7.32 (s, 1H), 7.26 (dd, J=8.3, 1.4 Hz, 1H), 7.17 (dd, J=12.1, 4.6 Hz, 2H), 6.94 (s, 1H), 6.12 (q, J=6.5 Hz, 1H), 4.90 (s, 1H), 3.50 (td, J=10.5, 5.2 Hz, 1H), 2.73 (d, J=16.3 Hz, 1H), 2.53 (m, 1H), 2.29-2.06 (m, 2H), 2.02-1.78 (m, 6H), 1.66 (m, 4H), 1.49-1.22 (m, 3H), 1.13 (m, 1H).

### Example 81 (P1 and P2)

### 4-[[(7R)-1-[2-[(1S)-1-(2,2-difluoro-1,3-benzodioxol-5-yl)ethoxy]-6-fluoro-4-pyridyl]-3-(trifluoromethyl)-4,5,6,7-tetrahydroindazol-7-yl]oxy]bicyclo[2.2.2]octane-1-carboxylic acid

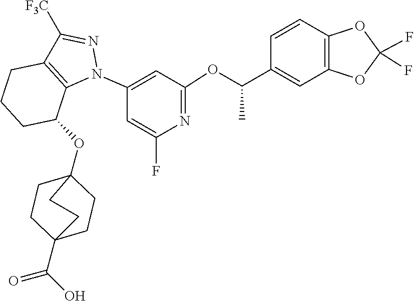 [SMILES: C[C@H](Oc1cc(-n2nc(C(F)(F)F)c3c2[C@H](OC24CCC(C(=O)O)(CC2)CC4)CCC3)cc(F)n1)c1ccc2c(c1)OC(F)(F)O2]

### 4-[[(7S)-1-[2-[(1S)-1-(2,2-difluoro-1,3-benzodioxol-5-yl)ethoxy]-6-fluoro-4-pyridyl]-3-(trifluoromethyl)-4,5,6,7-tetrahydroindazol-7-yl]oxy]bicyclo[2.2.2]octane-1-carboxylic acid

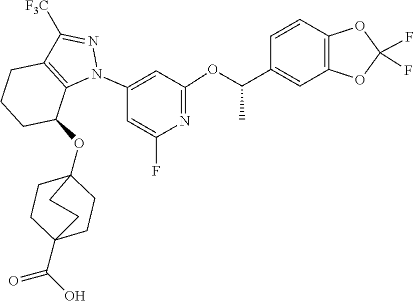 [SMILES: C[C@H](Oc1cc(-n2nc(C(F)(F)F)c3c2[C@@H](OC24CCC(C(=O)O)(CC2)CC4)CCC3)cc(F)n1)c1ccc2c(c1)OC(F)(F)O2]

P1: LCMS (Method 2): Retention time=2.01 min. MS (ESI) m/z 652.0 [M−H]⁺.

¹H NMR (400 MHZ, MeOH-d4) δ:7.34 (d, J=1.4 Hz, 1H), 7.29 (dd, J=8.3, 1.5 Hz, 1H), 7.16 (dd, J=14.4, 4.7 Hz, 2H), 6.87 (s, 1H), 6.17 (q, J=6.5 Hz, 1H), 5.05 (t, J=3.7 Hz, 1H), 2.71 (d, J=16.3 Hz, 1H), 2.60-2.46 (m, 1H), 2.10 (dd, J=12.4, 5.6 Hz, 1H), 2.02-1.90 (m, 1H), 1.81 (m, 8H), 1.70-1.56 (m, 9H).

P2: LCMS (Method 2): Retention time=2.02 min. MS (ESI) m/z 652.0 [M−H]⁺.

¹H NMR (400 MHZ, MeOH-d4) δ:7.36 (d, J=1.4 Hz, 1H), 7.29 (dd, J=8.3, 1.5 Hz, 1H), 7.18 (d, J=8.3 Hz, 1H), 7.12 (d, J=1.0 Hz, 1H), 6.87 (s, 1H), 6.17 (q, J=6.5 Hz, 1H), 5.10 (t, J=3.6 Hz, 1H), 2.71 (d, J=16.2 Hz, 1H), 2.62-2.44 (m, 1H), 2.08 (dd, J=12.3, 5.6 Hz, 1H), 1.99-1.75 (m, 9H), 1.62 (m, 9H).

### Example 82 (P1 and P2)

### 4-[[(7S)-1-[5-[(1S)-1-(2,2-difluoro-1,3-benzodioxol-5-yl)ethoxy]-6-fluoro-3-pyridyl]-3-(trifluoromethyl)-4,5,6,7-tetrahydroindazol-7-yl]oxymethyl]cyclohexanecarboxylic acid

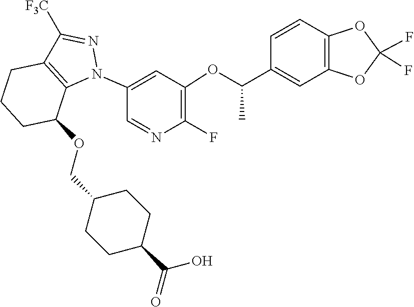 [SMILES: C[C@H](Oc1cc(-n2nc(C(F)(F)F)c3c2[C@@H](OC[C@H]2CC[C@H](C(=O)O)CC2)CCC3)cnc1F)c1ccc2c(c1)OC(F)(F)O2]

### 4-[[(7R)-1-[5-[(1S)-1-(2,2-difluoro-1,3-benzodioxol-5-yl)ethoxy]-6-fluoro-3-pyridyl]-3-(trifluoromethyl)-4,5,6,7-tetrahydroindazol-7-yl]oxymethyl]cyclohexanecarboxylic acid

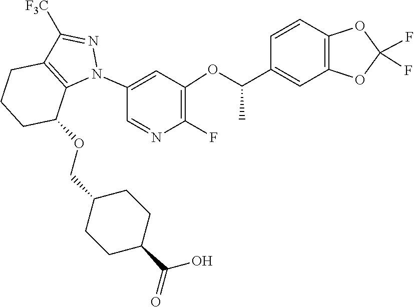 [SMILES: C[C@H](Oc1cc(-n2nc(C(F)(F)F)c3c2[C@H](OC[C@H]2CC[C@H](C(=O)O)CC2)CCC3)cnc1F)c1ccc2c(c1)OC(F)(F)O2]

P1: LCMS (Method2): LC retention time 1.87, MS (ESI): m/z 642 [M+H]⁺.

¹H NMR (400 MHZ, MeOH-d4) δ 7.99 (d, J=1.8 Hz, 1H), 7.69 (dd, J=8.6, 2.1 Hz, 1H), 7.34 (d, J=1.3 Hz, 1H), 7.25 (dt, J=18.0, 9.0 Hz, 1H), 7.18 (d, J=8.3 Hz, 1H), 5.64 (q, J=6.3 Hz, 1H), 4.54 (t, J=3.7 Hz, 1H), 3.31 (dd, J=3.3, 1.6 Hz, 1H), 3.04-2.89 (m, 1H), 2.72 (d, J=16.3 Hz, 1H), 2.60-2.47 (m, 1H), 2.07 (ddd, J=12.2, 7.9, 3.6 Hz, 2H), 1.79 (ddd, J=22.4, 14.7, 7.2 Hz, 5H), 1.67 (t, J=12.2 Hz, 3H), 1.49 (dd, J=34.1, 12.8 Hz, 2H), 1.36-1.16 (m, 3H), 0.87-0.67 (m, 2H).

P2: LCMS (Method2): LC retention time=1.87, MS (ESI): m/z 642 [M+H]⁺.

¹H NMR (400 MHZ, MeOH-d4) δ 7.89 (t, J=1.9 Hz, 1H), 7.68 (dd, J=8.6, 2.1 Hz, 1H), 7.38 (t, J=8.6 Hz, 1H), 7.33 (dd, J=8.3, 1.5 Hz, 1H), 7.24 (d, J=8.3 Hz, 1H), 5.61 (q, J=6.3 Hz, 1H), 4.16 (t, J=3.7 Hz, 1H), 3.12 (dd, J=8.6, 5.6 Hz, 1H), 2.77-2.61 (m, 1H), 2.59-2.44 (m, 1H), 2.38 (dd, J=8.5, 6.5 Hz, 1H), 2.13-1.97 (m, 2H), 1.92-1.62 (m, 8H), 1.48-1.37 (m, 1H), 1.37-1.17 (m, 3H), 1.16-1.02 (m, 1H), 0.76-0.50 (m, 2H).

### Example 83 (P1 and P2)

### 3-[[(7S)-1-[6-[(1S)-1-(2,2,6-trifluoro-1,3-benzodioxol-5-yl)ethoxy]-2-pyridyl]-3-(trifluoromethyl)-4,5,6,7-tetrahydroindazol-7-yl]oxy]cyclobutanecarboxylic acid (P1)

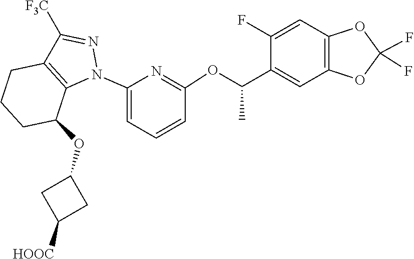 [SMILES: C[C@H](Oc1cccc(-n2nc(C(F)(F)F)c3c2[C@@H](O[C@H]2C[C@H](C(=O)O)C2)CCC3)n1)c1cc2c(cc1F)OC(F)(F)O2]

and

### 3-[[(7R)-1-[6-[(1S)-1-(2,2,6-trifluoro-1,3-benzodioxol-5-yl)ethoxy]-2-pyridyl]-3-(trifluoromethyl)-4,5,6,7-tetrahydroindazol-7-yl]oxy]cyclobutanecarboxylic acid (P2)

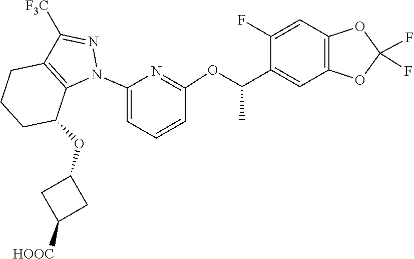 [SMILES: C[C@H](Oc1cccc(-n2nc(C(F)(F)F)c3c2[C@H](O[C@H]2C[C@H](C(=O)O)C2)CCC3)n1)c1cc2c(cc1F)OC(F)(F)O2]

P1: LCMS (Method 2): Retention time=1.92 min.: find peak 600.0 [M+H]⁺.

¹H NMR (400 MHZ, MeOH-d₄) δ:7.96-7.82 (m, 1H), 7.41 (d, J=5.7 Hz, 1H), 7.24 (dt, J=16.1, 7.9 Hz, 2H), 6.96 (t, J=11.8 Hz, 1H), 6.22 (q, J=6.5 Hz, 1H), 4.56 (t, J=3.6 Hz, 1H), 3.79-3.59 (m, 1H), 2.72 (d, J=16.4 Hz, 1H), 2.53 (dt, J=9.7, 6.2 Hz, 2H), 2.04 (dq, J=7.6, 4.1 Hz, 1H), 1.91 (ddd, J=11.8, 9.7, 4.0 Hz, 3H), 1.82-1.53 (m, 6H), 1.38-1.12 (m, 1H).

P2: LCMS (Method 2): Retention time=1.92 min.: find peak 600.0 [M+H]⁺.

¹H NMR (400 MHZ, MeOH-d₄) δ:7.86 (t, J=8.0 Hz, 1H), 7.35-7.19 (m, 2H), 7.05 (d, J=9.1 Hz, 1H), 6.92 (d, J=8.2 Hz, 1H), 6.34 (q, J=6.6 Hz, 1H), 5.50 (d, J=3.5 Hz, 1H), 4.41-4.26 (m, 1H), 2.81-2.60 (m, 2H), 2.60-2.33 (m, 2H), 2.24-1.99 (m, 3H), 1.99-1.60 (m, 7H).

### Example 84 (P1 and P2)

### 3-[[(7S)-1-[5-fluoro-6-[(1S)-1-(2,2,6-trifluoro-1,3-benzodioxol-5-yl)ethoxy]-2-pyridyl]-3-(trifluoromethyl)-4,5,6,7-tetrahydroindazol-7-yl]oxy]cyclobutanecarboxylic acid (P1)

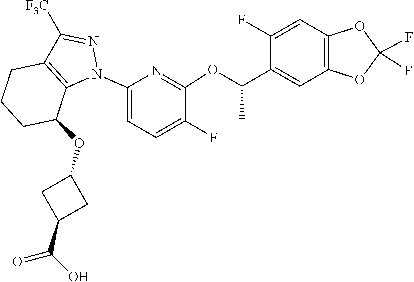 [SMILES: C[C@H](Oc1nc(-n2nc(C(F)(F)F)c3c2[C@@H](O[C@H]2C[C@H](C(=O)O)C2)CCC3)ccc1F)c1cc2c(cc1F)OC(F)(F)O2]

### 3-[[(7R)-1-[5-fluoro-6-[(1S)-1-(2,2,6-trifluoro-1,3-benzodioxol-5-yl)ethoxy]-2-pyridyl]-3-(trifluoromethyl)-4,5,6,7-tetrahydroindazol-7-yl]oxy]cyclobutanecarboxylic acid (P2)

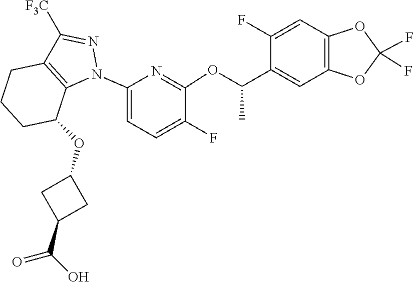 [SMILES: C[C@H](Oc1nc(-n2nc(C(F)(F)F)c3c2[C@H](O[C@H]2C[C@H](C(=O)O)C2)CCC3)ccc1F)c1cc2c(cc1F)OC(F)(F)O2]

P1: LCMS (Method 2): Retention time=1.96 min. MS (ESI) m/z 616.2 [M−H]⁻.

Chiral Method: Column Name: (R,R) Whelk-O1 4.6*100 mm 3.5 μm.

Solvent: MeOH [1% NH₃ (7M in MeOH)]; Find at 1.527 min; ee: 100%

¹H NMR (400 MHZ, MeOH-d₄) δ 7.77 (dd, J=9.3, 8.5 Hz, 1H), 7.42 (d, J=5.7 Hz, 1H), 7.25 (dd, J=12.6, 5.8 Hz, 2H), 6.29 (q, J=6.5 Hz, 1H), 4.46 (t, J=3.7 Hz, 1H), 3.63 (p, J=6.7 Hz, 1H), 2.78-2.65 (m, 1H), 2.61-2.47 (m, 2H), 2.03 (dq, J=7.6, 4.1 Hz, 1H), 1.93-1.82 (m, 3H), 1.81-1.63 (m, 6H), 1.37-1.27 (m, 1H).

P2: LCMS (Method 2): Retention time=1.96 min. MS (ESI) m/z 616.2 [M−H]⁻.

Chiral Method: Column Name: (R,R) Whelk-O1 4.6*100 mm 3.5 μm Solvent: EtOH [1% NH₃ (7M in MeOH)]; Find at 1.772 min; ee: 96.45%

¹H NMR (400 MHZ, MeOH-d₄) δ 7.72 (dd, J=9.3, 8.6 Hz, 1H), 7.33-7.25 (m, 2H), 7.06 (d, J=9.1 Hz, 1H), 6.42 (q, J=6.6 Hz, 1H), 5.38 (t, J=3.4 Hz, 1H), 4.32 (p, J=7.2 Hz, 1H), 2.77-2.66 (m, 2H), 2.59-2.47 (m, 1H), 2.41 (ddd, J=11.1, 7.4, 4.3 Hz, 1H), 2.20 (ddd, J=11.4, 7.4, 4.5 Hz, 1H), 2.14-2.03 (m, 2H), 1.95-1.64 (m, 7H).

### Example 85 (P1 and P2)

### 3-[3-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one (P1)

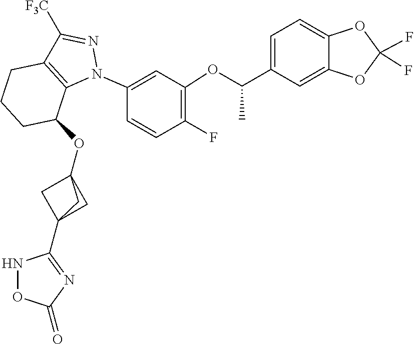 [SMILES: C[C@H](Oc1cc(-n2nc(C(F)(F)F)c3c2[C@@H](OC24CC(c5nc(=O)o[nH]5)(C2)C4)CCC3)ccc1F)c1ccc2c(c1)OC(F)(F)O2]

and

### 3-[3-[[(7R)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one (P2)

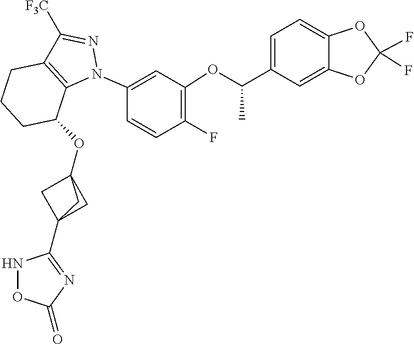 [SMILES: C[C@H](Oc1cc(-n2nc(C(F)(F)F)c3c2[C@H](OC24CC(c5nc(=O)o[nH]5)(C2)C4)CCC3)ccc1F)c1ccc2c(c1)OC(F)(F)O2]

### Step 1. Synthesis of 3-[[1-(3-bromo-4-fluoro-phenyl)-3-(trifluoromethyl)-4,5,6,7-tetrahydroindazol-7-yl]oxy]bicyclo[1.1.1]pentane-1-carboxamide

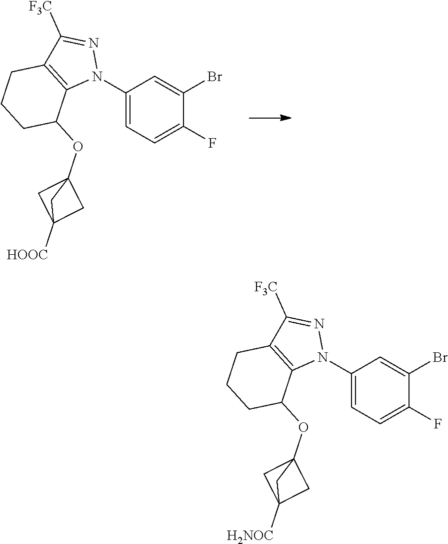 [SMILES: O=C(O)C12CC(OC3CCCc4c(C(F)(F)F)nn(-c5ccc(F)c(Br)c5)c43)(C1)C2 ; NC(=O)C12CC(OC3CCCc4c(C(F)(F)F)nn(-c5ccc(F)c(Br)c5)c43)(C1)C2]

To a solution of 3-[[1-(3-bromo-4-fluoro-phenyl)-3-(trifluoromethyl)-4,5,6,7-tetrahydroindazol-7-yl]oxy]bicyclo[1.1.1]pentane-1-carboxylic acid (500 mg, 1.02 mmol) in DMF (10 mL) was added ammonium chloride (66 mg, 1.23 mmol), O-(7-azabenzotriazol-1-yl)-N,N,N′,N′-tetramethyluronium hexafluorophosphate (369 mg, 1.53 mmol), and N,N-diisopropylethylamine (0.89 mL, 5.11 mmol). The reaction was stirred at 25° C. for 12 h. Then the mixture was diluted with water (10 mL) and extracted with EA (20 mL×3). The organic layers were combined, washed with brine, dried over Na₂SO₄, filtered, and concentrated to obtain 3-[[1-(3-bromo-4-fluoro-phenyl)-3-(trifluoromethyl)-4,5,6,7-tetrahydroindazol-7-yl]oxy]bicyclo[1.1.1]pentane-1-carboxamide (450 mg, 90.1%) as a white solid.

LCMS: Retention time=1.95 min. MS (ESI) m/z 488 and 490 [M+H]⁺.

### Step 2. Synthesis of 3-[[1-(3-bromo-4-fluoro-phenyl)-3-(trifluoromethyl)-4,5,6,7-tetrahydroindazol-7-yl]oxy]bicyclo[1.1.1]pentane-1-carbonitrile

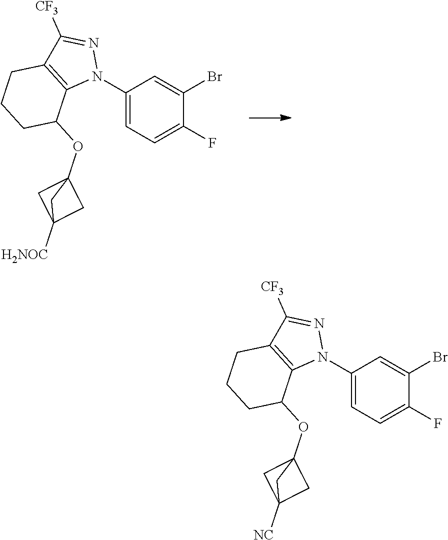 [SMILES: NC(=O)C12CC(OC3CCCc4c(C(F)(F)F)nn(-c5ccc(F)c(Br)c5)c43)(C1)C2 ; N#CC12CC(OC3CCCc4c(C(F)(F)F)nn(-c5ccc(F)c(Br)c5)c43)(C1)C2]

To a solution of 3-[[1-(3-bromo-4-fluoro-phenyl)-3-(trifluoromethyl)-4,5,6,7-tetrahydroindazol-7-yl]oxy]bicyclo[1.1.1]pentane-1-carboxamide (400 mg, 0.81 mmol) in DMF (8 mL) was added cyanuric chloride (66 mg, 3.28 mmol). The reaction was stirred at 25° C. for 2 h. Then the mixture was diluted with water (10 mL) and extracted with ethyl acetate (20 mL×3). The organic phases were combined, washed with brine, dried over Na₂SO₄ and concentrated in vacuum. The residue was purified by column chromatography on silica gel (0-100% ethyl acetate in heptane) to yield 3-[[1-(3-bromo-4-fluoro-phenyl)-3-(trifluoromethyl)-4,5,6,7-tetrahydroindazol-7-yl]oxy]bicyclo[1.1.1]pentane-1-carbonitrile (380 mg, 98.6%) as a yellow oil.

LCMS (Method 1): Retention time=2.26 min. MS (ESI) m/z 470.1, 472.1 [M+H]⁺.

### Step 3. Synthesis of 3-[[1-(4-fluoro-3-hydroxy-phenyl)-3-(trifluoromethyl)-4,5,6,7-tetrahydroindazol-7-yl]oxy]bicyclo[1.1.1]pentane-1-carbonitrile

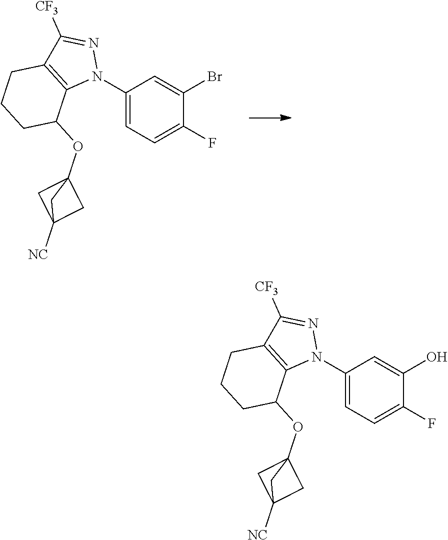 [SMILES: N#CC12CC(OC3CCCc4c(C(F)(F)F)nn(-c5ccc(F)c(Br)c5)c43)(C1)C2 ; N#CC12CC(OC3CCCc4c(C(F)(F)F)nn(-c5ccc(F)c(O)c5)c43)(C1)C2]

To a solution of 3-[[1-(3-bromo-4-fluoro-phenyl)-3-(trifluoromethyl)-4,5,6,7-tetrahydroindazol-7-yl]oxy]bicyclo[1.1.1]pentane-1-carbonitrile (350 mg, 0.74 mmol) in 1,4-dioxane (10 mL) were added water (67 mg, 3.72 mmol), cesium carbonate (727 mg, 2.23 mmol) and Rockphos-Pd-G3 (35 mg). The reaction was stirred at 100° C. for 0.5 h under N₂. Then the solid was filtered off. The filtrate was concentrated in vacuum. The residue was purified by column chromatography (0-100% ethyl acetate in heptane) to yield 3-[[1-(4-fluoro-3-hydroxy-phenyl)-3-(trifluoromethyl)-4,5,6,7-tetrahydroindazol-7-yl]oxy]bicyclo[1.1.1]pentane-1-carbonitrile (300 mg, 98.9% yield) as a yellow oil. LCMS (Method 2): Retention time=1.95 min. MS (ESI) m/z 408.0 [M+H]⁺.

### Step 4. Synthesis of 3-[[1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]bicyclo[1.1.1]pentane-1-carbonitrile

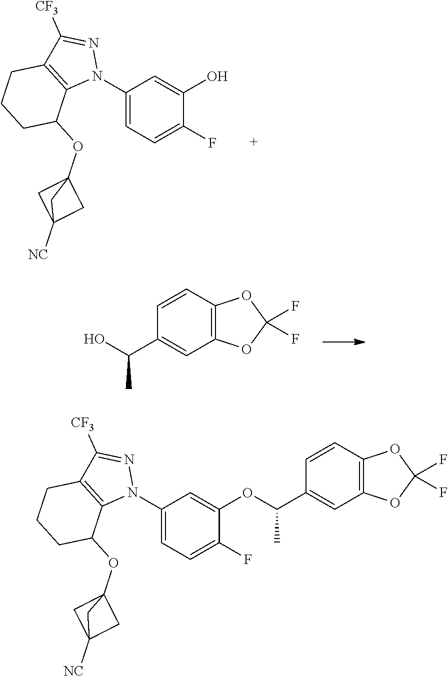 [SMILES: N#CC12CC(OC3CCCc4c(C(F)(F)F)nn(-c5ccc(F)c(O)c5)c43)(C1)C2 ; C[C@@H](O)c1ccc2c(c1)OC(F)(F)O2 ; C[C@H](Oc1cc(-n2nc(C(F)(F)F)c3c2C(OC24CC(C#N)(C2)C4)CCC3)ccc1F)c1ccc2c(c1)OC(F)(F)O2]

To a solution of 3-[[1-(4-fluoro-3-hydroxy-phenyl)-3-(trifluoromethyl)-4,5,6,7-tetrahydroindazol-7-yl]oxy]bicyclo[1.1.1]pentane-1-carbonitrile (200 mg, 0.49 mmol) in THF (5 mL) were added (1R)-1-(2,2-difluoro-1,3-benzodioxol-5-yl)ethanol (Intermediate C-1R) (119 mg, 0.59 mmol) and triphenylphosphine (258 mg, 0.98 mmol). Then the mixture was cooled to 0° C. under N₂, and diethyl azodicarboxylate (0.09 mL, 0.59 mmol) was added dropwise at 0° C. over 10 min. The reaction was then stirred at rt for 1 h. The mixture was concentrated and the crude product was purified by flash column chromatography (ethyl acetate in PE: 0-25% to get 3-[[1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]bicyclo[1.1.1]pentane-1-carbonitrile (260 mg, 89.5%) as a white solid.

LCMS (Method 1): Retention time=2.38 min. MS (ESI) m/z 592.0 [M+H]⁺.

### Step 5. Synthesis of 3-[[1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-N-hydroxy-bicyclo[1.1.1]pentane-1-carboxamidine

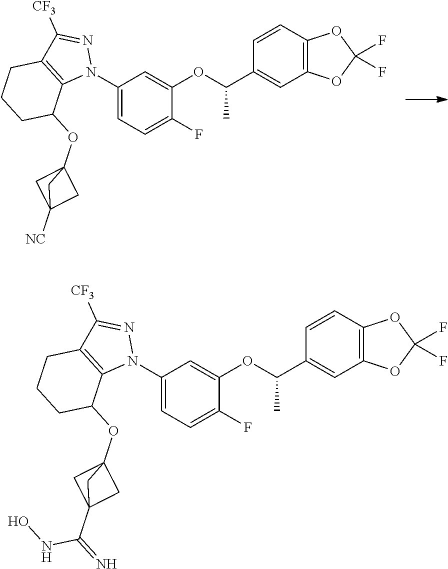 [SMILES: C[C@H](Oc1cc(-n2nc(C(F)(F)F)c3c2C(OC24CC(C#N)(C2)C4)CCC3)ccc1F)c1ccc2c(c1)OC(F)(F)O2 ; C[C@H](Oc1cc(-n2nc(C(F)(F)F)c3c2C(OC24CC(C(=N)NO)(C2)C4)CCC3)ccc1F)c1ccc2c(c1)OC(F)(F)O2]

To a solution of 3-[[1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]bicyclo[1.1.1]pentane-1-carbonitrile (260 mg, 0.44 mmol) in ethanol (3 mL) was added hydroxylamine (3.0 mL, 0.44 mmol). The reaction was stirred at 80° C. for 1 h. The mixture was concentrated and the crude was purified by flash column chromatography (ethyl acetate in PE.) to get 3-[1-[3-[[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-N-hydroxy-bicyclo[1.1.1]pentane-1-carboxamidine (200 mg, 72.8% yield) as a white solid.

LCMS (Method 1): Retention time=2.03 min. MS (ESI) m/z 625.1 [M+H]⁺.

### Step 6. Synthesis of 3-[3-[[(7R)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one and 3-[3-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one

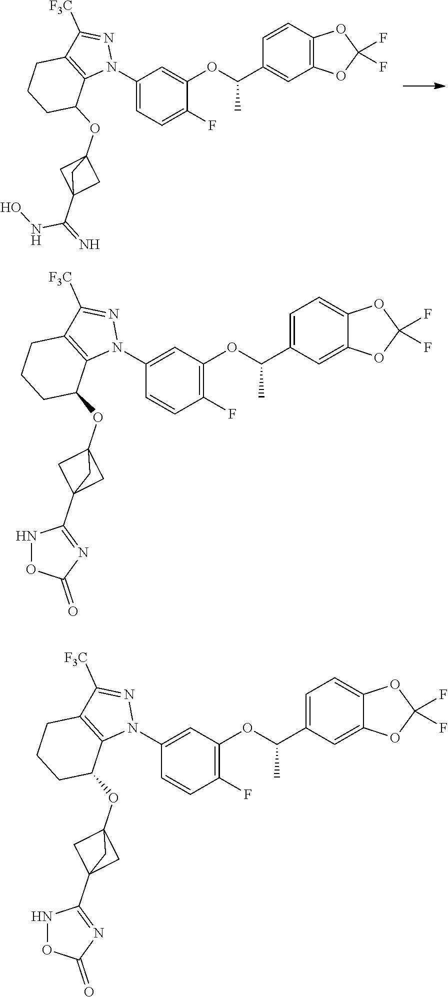

To a solution of 3-[[1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-N-hydroxy-bicyclo[1.1.1]pentane-1-carboxamidine and methyl thiohypofluorite (90 mg, 0.13 mmol) in ethyl acetate (10 mL) was added N,N-carbonyldiimidazole (106 mg, 0.65 mmol). The reaction was stirred at 65° C. for 12 h. The mixture was concentrated and the crude was purified by flash column chromatography (ethyl acetate in PE: 0-15% for 20 min) to get the product (90 mg) which was separated by SFC to get 3-[3-[[(7R)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one (P1) (38 mg, 44.9% yield) and 3-[3-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one (P2) (38 mg, 44.3% yield). Both products are white solids.

P1: LCMS (Method 1): Retention time=1.85 min. MS (ESI) m/z 651 [M+H]⁺.

¹H NMR (400 MHZ, MeOH-d₄) δ:7.35 (d, J=1.4 Hz, 1H), 7.33-7.24 (m, 2H), 7.23-7.17 (m, 2H), 7.15 (d, J=3.8 Hz, 1H), 5.58 (dq, J=12.7, 6.4 Hz, 1H), 4.52 (t, J=3.2 Hz, 1H), 2.75 (d, J=16.3 Hz, 1H), 2.60-2.43 (m, 1H), 2.12-1.94 (m, 4H), 1.96-1.71 (m, 6H), 1.66 (d, J=6.3 Hz, 3H).

P2: LCMS (Method 1): Retention time=1.85 min. MS (ESI) m/z 651 [M+H]⁺.

¹H NMR (400 MHZ, MeOH-d₄) δ:7.36-7.17 (m, 4H), 7.17-7.03 (m, 2H), 5.59 (q, J=6.3 Hz, 1H), 4.77 (d, J=3.4 Hz, 1H), 2.74 (d, J=16.4 Hz, 1H), 2.51 (dd, J=16.2, 9.3 Hz, 1H), 2.13-1.96 (m, 4H), 1.87 (ddd, J=39.4, 16.5, 6.4 Hz, 6H), 1.66 (d, J=6.4 Hz, 3H).

### Example 86 (P1 and P2)

### 3-[3-[[(7S)-1-[4-fluoro-3-[(1S)-1-(2,2,6-trifluoro-1,3-benzodioxol-5-yl)ethoxy]phenyl]-3-(trifluoromethyl)-4,5,6,7-tetrahydroindazol-7-yl]oxy]-1-bicyclo[1.1.1]pentanyl]-2H-1,2,4-oxadiazol-5-one

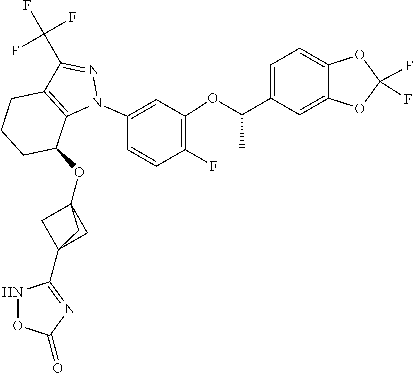 [SMILES: C[C@H](Oc1cc(-n2nc(C(F)(F)F)c3c2[C@@H](OC24CC(c5nc(=O)o[nH]5)(C2)C4)CCC3)ccc1F)c1ccc2c(c1)OC(F)(F)O2]

and

### 3-[3-[[(7R)-1-[4-fluoro-3-[(1S)-1-(2,2,6-trifluoro-1,3-benzodioxol-5-yl)ethoxy]phenyl]-3-(trifluoromethyl)-4,5,6,7-tetrahydroindazol-7-yl]oxy]-1-bicyclo[1.1.1]pentanyl]-2H-1,2,4-oxadiazol-5-one

Example 86 (P1 and P2) was synthesized similarly to Example 85 (P1 and P2).

P1: LCMS (Method 1): Retention time=1.85 min. MS (ESI) m/z 669.0 [M+H]⁺.

¹H NMR (400 MHZ, MeOH-d₄) δ:7.40 (dd, J=10.3, 5.6 Hz, 1H), 7.32 (dd, J=10.7, 8.7 Hz, 1H), 7.24-7.08 (m, 3H), 5.81 (q, J=6.3 Hz, 1H), 4.65 (t, J=3.3 Hz, 1H), 2.76 (d, J=16.6 Hz, 1H), 2.53 (ddd, J=16.2, 9.7, 6.3 Hz, 1H), 2.16-1.93 (m, 7H), 1.81 (ddd, J=25.8, 9.5, 3.2 Hz, 3H), 1.69 (d, J=6.4 Hz, 3H).

P2: LCMS (Method 1): Retention time=1.85 min. MS (ESI) m/z 669.0 [M+H]⁺.

¹H NMR (400 MHZ, MeOH-d₄) δ:7.40 (dd, J=10.2, 5.7 Hz, 1H), 7.32 (dd, J=10.7, 8.6 Hz, 1H), 7.24-7.03 (m, 3H), 5.82 (q, J=6.3 Hz, 1H), 4.78 (d, J=3.5 Hz, 1H), 2.75 (d, J=16.2 Hz, 1H), 2.65-2.35 (m, 1H), 2.15-2.02 (m, 4H), 2.02-1.91 (m, 3H), 1.93-1.74 (m, 3H), 1.69 (d, J=6.4 Hz, 3H).

### Example 87

### 4-[[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]oxy]benzoic acid

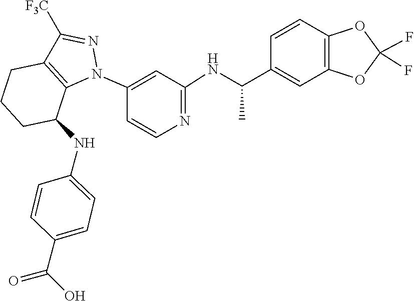 [SMILES: C[C@H](Nc1cc(-n2nc(C(F)(F)F)c3c2[C@@H](Nc2ccc(C(=O)O)cc2)CCC3)ccn1)c1ccc2c(c1)OC(F)(F)O2]

### Step 1. Synthesis of(S)-4-bromo-N-(1-(2,2-difluorobenzo[d][1,3]dioxol-5-yl)ethyl)pyridine-2-amine

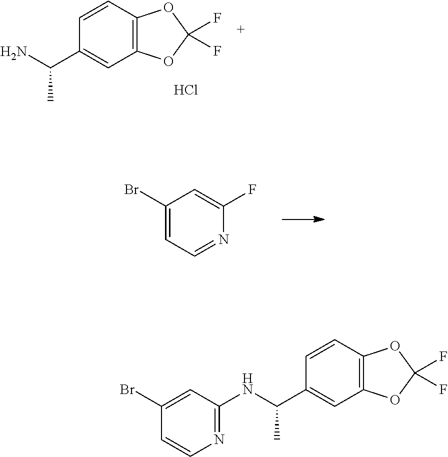 [SMILES: C[C@H](N)c1ccc2c(c1)OC(F)(F)O2 ; Cl ; Fc1cc(Br)ccn1 ; C[C@H](Nc1cc(Br)ccn1)c1ccc2c(c1)OC(F)(F)O2]

To a solution of (1S)-1-(2,2-difluoro-1,3-benzodioxol-5-yl)ethanamine hydrochloride (Intermediate C-6a) (1000 mg, 4.21 mmol) in DMSO (15 mL) were added 4-bromo-2-fluoro-pyridine (2222 mg, 12.60 mmol) and cesium fluoride (1918 mg, 12.60 mmol). The reaction was stirred at 100° C. for 8 h. Water (20 mL) was added and the aqueous solution was extracted with ethyl acetate (20 mL×3). The organic solution was dried (Na₂SO₄), filtered, and concentrated. The crude was purified by flash silica gel column chromatography (eluting with ethyl acetate in petroleum ether from 0% to 25%) to afford the title compound (1000 mg, 63.2% yield) as a yellow oil.

LCMS (Method 2): Retention time=2.16 min. MS (ESI) m/z 357.0, 359.0 [M+H]⁺.

### Step 2. Synthesis of(S)-(2-((1-(2,2-difluorobenzo[d][1,3]dioxol-5-yl)ethyl)amino)pyridine-4-yl) boronic acid

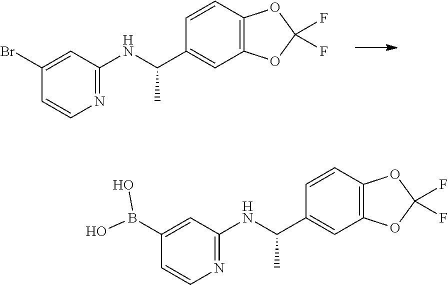 [SMILES: C[C@H](Nc1cc(Br)ccn1)c1ccc2c(c1)OC(F)(F)O2 ; C[C@H](Nc1cc(B(O)O)ccn1)c1ccc2c(c1)OC(F)(F)O2]

To a solution of 4-bromo-N-[(1S)-1-(2,2-difluoro-1,3-benzodioxol-5-yl)ethyl]pyridine-2-amine (1000 mg, 2.80 mmol) in 1,4-dioxane (15 mL) were added bis(pinacolato)diboron (853 mg, 3.36 mmol), KOAc (550 mg, 5.60 mmol) and Pd(dppf)Cl₂ (102 mg, 0.14 mmol) under N₂. The reaction was stirred at 80° C. for 8 h. The reaction mixture was concentrated under vacuum to give a residue, which was purified by flash silica gel column chromatography (eluting with 0% to 100% ethyl acetate in petroleum ether) followed by prep-HPLC (TFA condition) to obtain the title compound (750 mg, 81.5% yield) as a brown solid.

LCMS (Method 1): Retention time=1.53 min. MS (ESI) m/z 323.0 [M+H]⁺.

### Step 3. Synthesis of(S)-1-(2-((1-(2,2-difluorobenzo[d][1,3]dioxol-5-yl)ethyl)amino)pyridine-4-yl)-3-(trifluoromethyl)-1,4,5,6-tetrahydro-7H-indazol-7-one

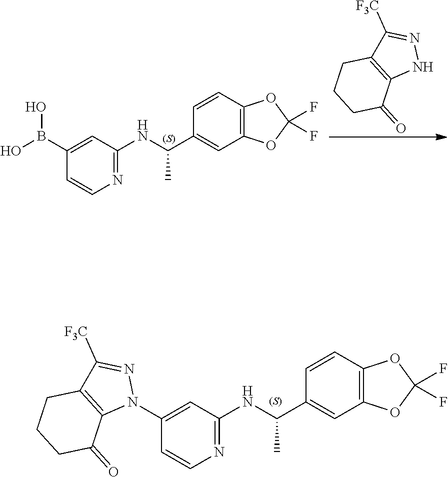

To a solution of [2-[[(1S)-1-(2,2-difluoro-1,3-benzodioxol-5-yl)ethyl]amino]-4-pyridyl]boronic acid (750 mg, 2.33 mmol), 3-(trifluoromethyl)-1,4,5,6-tetrahydroindazol-7-one (475 mg, 2.33 mmol) and copper (II) acetate (211 mg, 1.16 mmol) in DCE (15 mL) was added triethylamine (0.36 mL, 2.56 mmol) under 02. The reaction was stirred at room temperature for 8 h. The reaction mixture was concentrated in vacuo to give a residue, which was purified by flash silica gel column chromatography (eluting with 0% to 20% ethyl acetate in petroleum ether) to afford the title compound (85 mg, 7.5% yield) as a yellow solid.

LCMS (Method 2): Retention time=2.22 min. MS (ESI) m/z 481.0 [M+H]⁺.

### Step 4. Synthesis of R-1-(2-(((S)-1-(2,2-difluorobenzo[d][1,3]dioxol-5-yl)ethyl)amino)pyridine-4-yl)-3-(trifluoromethyl)-4,5,6,7-tetrahydro-1H-indazol-7-ol

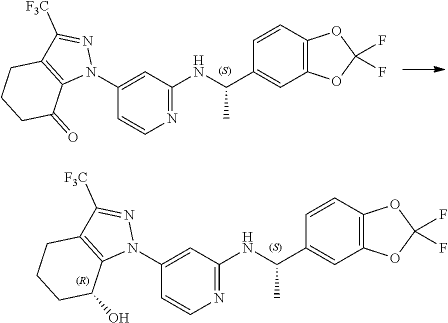

To a solution of 1-[2-[[(1S)-1-(2,2-difluoro-1,3-benzodioxol-5-yl)ethyl]amino]-4-pyridyl]-3-(trifluoromethyl)-5,6-dihydro-4H-indazol-7-one (85 mg, 0.18 mmol) in IPA (5 mL) were added RuCl(p-cymene)[(R,R)-Ts-DPEN] (5 mg) under N₂, followed by triethylamine (0.30 mL, 2.15 mmol) and formic acid (0.20 mL, 5.21 mmol) portion wise. The reaction was stirred at 45° C. for 2 h. The reaction mixture was concentrated in vacuum to give a residue, which was purified by flash silica gel column chromatography (ethyl acetate in petroleum ether from 0% to 15%) to afford the title compound (80 mg, 91.8% yield) as a brown oil.

LCMS (Method 2): Retention time=2.16 min. MS (ESI) m/z 483.0 [M+H]⁺.

### Step 5. Synthesis of methyl 4-(((S)-1-(2-(((S)-1-(2,2-difluorobenzo[d][1,3]dioxol-5-yl)ethyl)amino)pyridine-4-yl)-3-(trifluoromethyl)-4,5,6,7-tetrahydro-1H-indazol-7-yl)oxy)benzoate

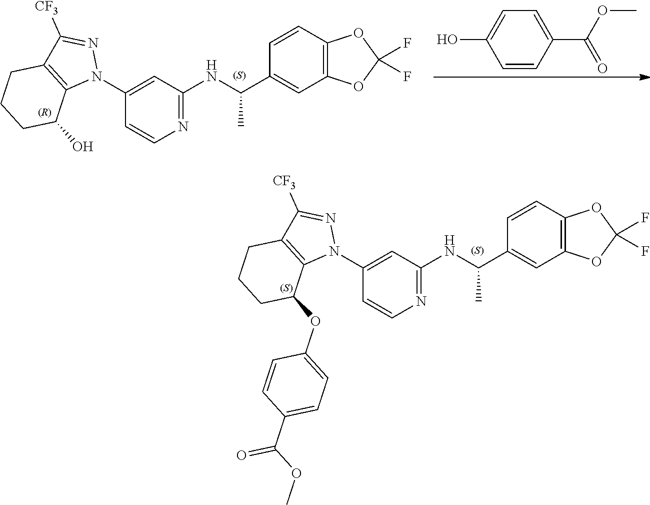

To a solution of (7R)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-ol (80 mg, 0.17 mmol) in THF (5 mL) was added methyl 4-hydroxybenzoate (28 mg, 0.18 mmol) under N₂. The reaction was stirred at 0° C. for 15 min. Then, to the reaction mixture were added triphenylphosphine (130 mg, 0.50 mmol) and diethyl azodicarboxylate (0.039 mL, 0.25 mmol) at 0° C. The reaction was stirred at room temperature for 1 h. The reaction mixture was concentrated in vacuo to give a residue, which was purified by flash silica gel column chromatography (ethyl acetate in petroleum ether from 0% to 15%) to obtain the title compound (35 mg, 33.5% yield) as a yellow solid.

LCMS (Method 2): Retention time=2.49 min. MS (ESI) m/z 617.1 [M+H]⁺.

### Step 6. Synthesis of 4-(((S)-1-(2-(((S)-1-(2,2-difluorobenzo[d][1,3]dioxol-5-yl)ethyl)amino)pyridine-4-yl)-3-(trifluoromethyl)-4,5,6,7-tetrahydro-1H-indazol-7-yl)oxy)benzoic acid

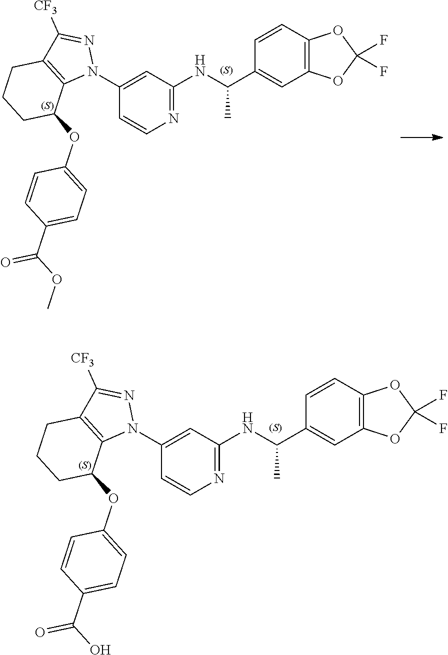

To a solution of methyl 4-[[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]oxy]benzoate (30 mg, 0.05 mmol) in methanol (1 mL), THF (1 mL) and water (0.50 mL) was added lithium hydroxide monohydrate (10 mg, 0.24 mmol). The reaction was stirred at room temperature for 2 h. The aqueous phase was acidified with aqueous HCl (2 M) until pH=5 and then extracted with ethyl acetate (5 mL×3). The crude product was purified by prep-HPLC (NH₄HCO₃ condition) to obtain the title compound (20 mg, 67.2% yield) as a white solid.

LCMS (Method 2): Retention time=1.73 min. MS (ESI) m/z 603.0 [M+H]⁺.

¹H NMR (400 MHZ, MeOH-d₄) δ 7.97 (d, J=8.8 Hz, 2H), 7.83 (d, J=5.6 Hz, 1H), 7.02 (dd, J=13.1, 8.6 Hz, 4H), 6.94 (d, J=8.3 Hz, 1H), 6.68 (d, J=7.5 Hz, 1H), 6.60 (s, 1H), 5.67 (s, 1H), 4.64 (d, J=6.8 Hz, 1H), 2.83 (d, J=15.8 Hz, 1H), 2.66-2.54 (m, 1H), 2.23 (d, J=13.8 Hz, 1H), 1.93 (dd, J=39.3, 14.2 Hz, 3H), 1.33 (d, J=6.9 Hz, 3H).

The example 88-92 were synthesized according to the procedure as Example 87

### Example 88

### 4-[[(7S)-1-[2-[[(1S)-1-(2,2-difluoro-1,3-benzodioxol-5-yl) propyl]amino]-4-pyridyl]-3-(trifluoromethyl)-4,5,6,7-tetrahydroindazol-7-yl]oxy]benzoic acid

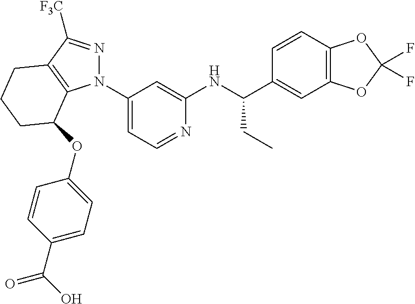 [SMILES: CC[C@H](Nc1cc(-n2nc(C(F)(F)F)c3c2[C@@H](Oc2ccc(C(=O)O)cc2)CCC3)ccn1)c1ccc2c(c1)OC(F)(F)O2]

LCMS (Method 2): Retention time=1.79 min. MS (ESI) m/z 617.1 [M+H]⁺.

¹H NMR (400 MHZ, MeOH-d₄) δ:7.96 (d, J=8.9 Hz, 2H), 7.83-7.74 (m, 1H), 7.11 (s, 1H), 7.05-6.95 (m, 4H), 6.72-6.64 (m, 2H), 5.77 (s, 1H), 4.59 (t, J=7.0 Hz, 1H), 2.84 (d, J=16.1 Hz, 1H), 2.68-2.55 (m, 1H), 2.28-2.16 (m, 1H), 2.01-1.86 (m, 3H), 1.78-1.66 (m, 2H), 0.88 (t, J=7.4 Hz, 3H).

### Example 89

### 4-[[(7S)-1-[2-[[(1S)-1-(2,2-difluoro-1,3-benzodioxol-5-yl)-2-methyl-propyl]amino]-4-pyridyl]-3-(trifluoromethyl)-4,5,6,7-tetrahydroindazol-7-yl]oxy]benzoic acid

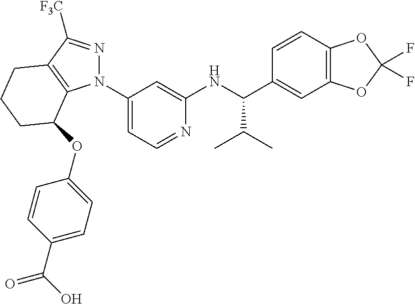 [SMILES: CC(C)[C@H](Nc1cc(-n2nc(C(F)(F)F)c3c2[C@@H](Oc2ccc(C(=O)O)cc2)CCC3)ccn1)c1ccc2c(c1)OC(F)(F)O2]

LCMS (Method 4): Retention time=1.81 min. MS (ESI) m/z 631.0 [M+H]⁺.

¹H NMR (400 MHZ, MeOH-d₄) δ:8.01 (d, J=8.7 Hz, 2H), 7.87 (d, J=5.6 Hz, 1H), 7.14-6.98 (m, 4H), 6.93 (d, J=8.2 Hz, 1H), 6.75-6.59 (m, 2H), 5.70 (s, 1H), 4.20 (s, 1H), 2.86 (d, J=16.3 Hz, 1H), 2.74-2.51 (m, 1H), 2.26 (d, J=13.5 Hz, 1H), 1.91 (dd, J=26.4, 19.7 Hz, 4H), 0.90 (d, J=6.7 Hz, 3H), 0.69 (d, J=6.7 Hz, 3H).

### Example 90

### 4-[[(7S)-1-[2-[[(S)-cyclopropyl-(2,2-difluoro-1,3-benzodioxol-5-yl)methyl]amino]-4-pyridyl]-3-(trifluoromethyl)-4,5,6,7-tetrahydroindazol-7-yl]oxy]benzoic acid

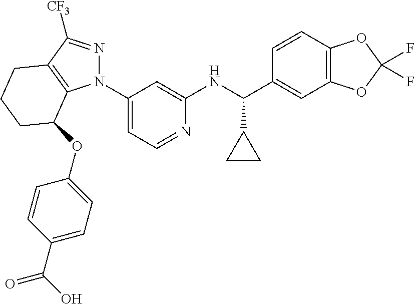 [SMILES: O=C(O)c1ccc(O[C@H]2CCCc3c(C(F)(F)F)nn(-c4ccnc(N[C@H](c5ccc6c(c5)OC(F)(F)O6)C5CC5)c4)c32)cc1]

LCMS (Method 4): Retention time=1.78 min. MS (ESI) m/z 629.1 [M+H]⁺.

¹H NMR (400 MHZ, MeOH-d₄) δ:8.01 (d, J=8.8 Hz, 2H), 7.86 (d, J=5.6 Hz, 1H), 7.05 (dd, J=8.1, 6.0 Hz, 4H), 6.98 (d, J=9.4 Hz, 1H), 6.70 (dd, J=5.7, 1.7 Hz, 1H), 6.59 (s, 1H), 5.67 (s, 1H), 3.87 (d, J=8.6 Hz, 1H), 2.85 (d, J=15.9 Hz, 1H), 2.61 (ddd, J=16.6, 10.5, 5.8 Hz, 1H), 2.24 (d, J=11.8 Hz, 1H), 1.89 (dd, J=38.1, 24.1 Hz, 3H), 1.1-0.97 (m, 1H), 0.57 (t, J=8.6 Hz, 1H), 0.48 (t, J=8.7 Hz, 1H), 0.24 (dt, J=14.3, 7.2 Hz, 2H).

### Example 91

### 4-[[(7S)-1-[6-[[(1S)-1-(2,2-difluoro-1,3-benzodioxol-5-yl)ethyl]amino]-2-pyridyl]-3-(trifluoromethyl)-4,5,6,7-tetrahydroindazol-7-yl]oxy]benzoic acid

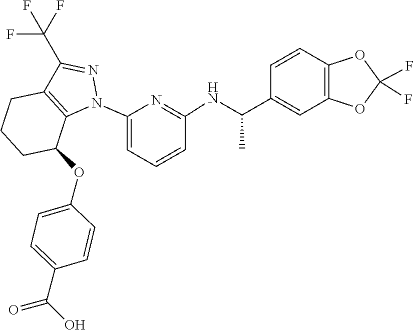 [SMILES: C[C@H](Nc1cccc(-n2nc(C(F)(F)F)c3c2[C@@H](Oc2ccc(C(=O)O)cc2)CCC3)n1)c1ccc2c(c1)OC(F)(F)O2]

LCMS (Method 4): Retention time=1.84 min. MS (ESI) m/z 603.1 [M+H]⁺.

¹H NMR (400 MHZ, MeOH-d₄) δ:7.88 (d, J=8.8 Hz, 2H), 7.43 (t, J=8.0 Hz, 1H), 7.09 (ddd, J=9.8, 5.8, 1.4 Hz, 3H), 6.90 (d, J=7.6 Hz, 1H), 6.83 (d, J=8.8 Hz, 2H), 6.23-6.10 (m, 2H), 4.52 (q, J=6.7 Hz, 1H), 2.85 (d, J=14.7 Hz, 1H), 2.62 (dd, J=16.5, 6.3 Hz, 1H), 2.25 (dd, J=9.4, 5.0 Hz, 1H), 1.95 (dt, J=19.6, 11.5 Hz, 3H), 1.24 (t, J=9.0 Hz, 3H).

### Example 92

### 4-[[(7S)-1-[6-[[(1S)-1-(2,2-difluoro-1,3-benzodioxol-5-yl)ethyl]amino]-5-fluoro-2-pyridyl]-3-(trifluoromethyl)-4,5,6,7-tetrahydroindazol-7-yl]oxy]benzoic acid

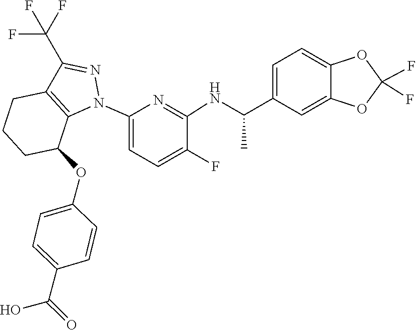 [SMILES: C[C@H](Nc1nc(-n2nc(C(F)(F)F)c3c2[C@@H](Oc2ccc(C(=O)O)cc2)CCC3)ccc1F)c1ccc2c(c1)OC(F)(F)O2]

LCMS (Method 4): Retention time=1.68 min. MS (ESI) m/z 619.1 [M−H]⁻.

¹H NMR (400 MHZ, MeOH-d₄) δ:7.68 (d, J=8.8 Hz, 2H), 7.27 (dd, J=13.4, 5.2 Hz, 1H), 7.20 (s, 1H), 7.13 (s, 2H), 6.65 (dd, J=8.2, 2.6 Hz, 1H), 6.38 (d, J=8.8 Hz, 2H), 5.58 (s, 1H), 5.01-4.95 (m, 1H), 2.83 (d, J=16.1 Hz, 1H), 2.62 (d, J=10.2 Hz, 1H), 2.11 (d, J=10.6 Hz, 1H), 1.89 (dd, J=23.4, 11.3 Hz, 3H), 1.41 (t, J=9.4 Hz, 3H).

### Example 93

### 4-[[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]amino]benzoic acid

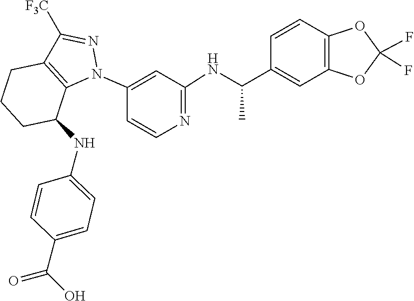 [SMILES: C[C@H](Nc1cc(-n2nc(C(F)(F)F)c3c2[C@@H](Nc2ccc(C(=O)O)cc2)CCC3)ccn1)c1ccc2c(c1)OC(F)(F)O2]

### Step 1. Synthesis of 1-(2-fluoropyridin-4-yl)-3-(trifluoromethyl)-1,4,5,6-tetrahydrospiro[indazole-7,2′-[1,3]dioxolane]

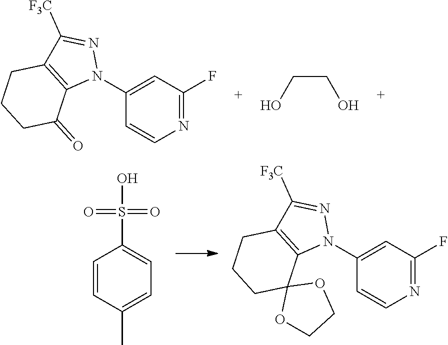

To a solution of 1-(2-fluoro-4-pyridyl)-3-(trifluoromethyl)-5,6-dihydro-4H-indazol-7-one (1.00 eq, 700 mg, 2.34 mmol) in toluene (10 mL) was added 4-methylbenzenesulfonic acid (0.200 eq, 81 mg, 0.468 mmol). The reaction was stirred at 110° C. for 2 h. Then water (30 mL) was added and the aqueous solution was extracted with EA (20 mL×2). The EA solution was washed with brine (20 mL), then concentrated in vacuum. The residue was purified by silica gel column chromatography (0-20% ethyl acetate in heptane) to yield 1′-(2-fluoro-4-pyridyl)-3′-(trifluoromethyl)spiro[1,3-dioxolane-2,7′-5,6-dihydro-4H-indazole] (450 mg, 1.31 mmol, 56.0% yield) as a yellow solid.

LC-MS (Method 4): Retention time=2.03 min. MS (ESI) m/z 344.0 [M+H]⁺.

### Step 2. Synthesis of 1-(2-fluoropyridin-4-yl)-3-(trifluoromethyl)-1,4,5,6-tetrahydrospiro[indazole-7,2′-[1,3]dioxolane]

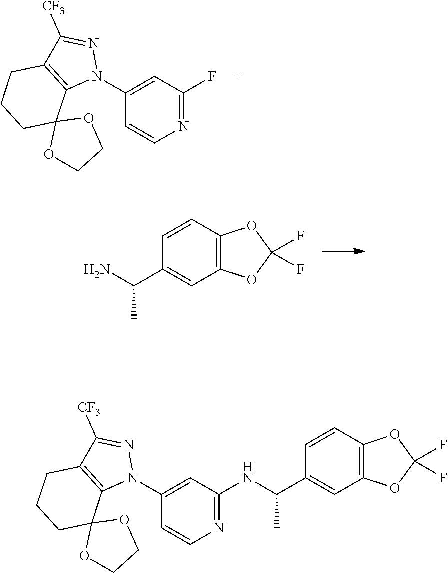 [SMILES: Fc1cc(-n2nc(C(F)(F)F)c3c2C2(CCC3)OCCO2)ccn1 ; C[C@H](N)c1ccc2c(c1)OC(F)(F)O2 ; C[C@H](Nc1cc(-n2nc(C(F)(F)F)c3c2C2(CCC3)OCCO2)ccn1)c1ccc2c(c1)OC(F)(F)O2]

To a solution of 1′-(2-fluoro-4-pyridyl)-3′-(trifluoromethyl)spiro[1,3-dioxolane-2,7′-5,6-dihydro-4H-indazole] (1.00 eq, 450 mg, 1.31 mmol) and (1S)-1-(2,2-difluoro-1,3-benzodioxol-5-yl)ethanamine (Intermediate C-6a) (1.50 eq, 396 mg, 1.97 mmol) in NMP (1 mL) was added N,N-diisopropylethylamine (2.50 eq, 0.57 mL, 3.28 mmol). The reaction was stirred at 160° C. for 24 h under N₂. Then water (30 mL) was added, extracted with EA (30 mL×2). The EA solution was washed with brine (20 mL) then concentrated in vacuo to yield N-[(1S)-1-(2,2-difluoro-1,3-benzodioxol-5-yl)ethyl]-4-[3′-(trifluoromethyl)spiro[1,3-dioxolane-2,7′-5,6-dihydro-4H-indazole]-1′-yl]pyridine-2-amine (400 mg, 0.763 mmol, 58.2% yield) as a yellow oil. This sample was used in the next reaction step without purification.

LC-MS (Method 4): Retention time=2.28 min. MS (ESI) m/z 525.0 [M+H]⁺.

### Step 3. Synthesis of(S)-1-(2-((1-(2,2-difluorobenzo[d][1,3]dioxol-5-yl)ethyl)amino)pyridine-4-yl)-3-(trifluoromethyl)-1,4,5,6-tetrahydro-7H-indazol-7-one

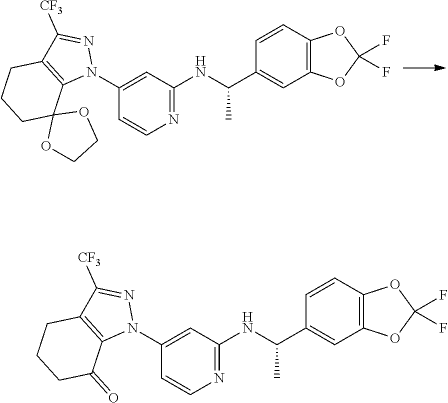 [SMILES: C[C@H](Nc1cc(-n2nc(C(F)(F)F)c3c2C2(CCC3)OCCO2)ccn1)c1ccc2c(c1)OC(F)(F)O2 ; C[C@H](Nc1cc(-n2nc(C(F)(F)F)c3c2C(=O)CCC3)ccn1)c1ccc2c(c1)OC(F)(F)O2]

The solution of N-[(1S)-1-(2,2-difluoro-1,3-benzodioxol-5-yl)ethyl]-4-[3′-(trifluoromethyl)spiro[1,3-dioxolane-2,7′-5,6-dihydro-4H-indazole]-1′-yl]pyridine-2-amine (1.00 eq, 400 mg, 0.763 mmol) in formic acid (68.4 eq, 2.0 mL, 52.1 mmol) was stirred at 0° C. under N₂ for 1 h. Then solvent was removed and EA (20 mL) was added. The mixture was concentrated under vacuum. The residue was purified by silica gel column chromatography (0-30% ethyl acetate in heptane) to yield 1-[2-[[(1S)-1-(2,2-difluoro-1,3-benzodioxol-5-yl)ethyl]amino]-4-pyridyl]-3-(trifluoromethyl)-5,6-dihydro-4H-indazol-7-one (350 mg, 0.729 mmol, 95.5% yield) as a yellow solid.

LC-MS (Method 4): Retention time=2.19 min. MS (ESI) m/z 481.0 [M+H]⁺.

### Step 4. Synthesis of (R)-1-(2-(((S)-1-(2,2-difluorobenzo[d][1,3]dioxol-5-yl)ethyl)amino)pyridine-4-yl)-3-(trifluoromethyl)-4,5,6,7-tetrahydro-1H-indazol-7-ol

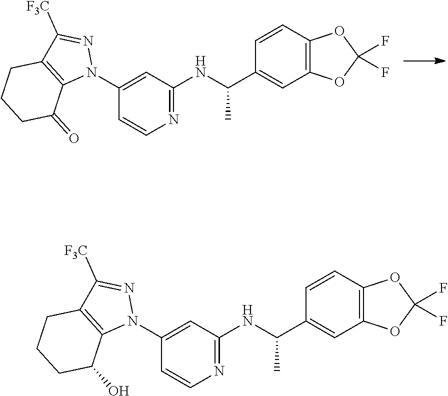 [SMILES: C[C@H](Nc1cc(-n2nc(C(F)(F)F)c3c2C(=O)CCC3)ccn1)c1ccc2c(c1)OC(F)(F)O2 ; C[C@H](Nc1cc(-n2nc(C(F)(F)F)c3c2[C@H](O)CCC3)ccn1)c1ccc2c(c1)OC(F)(F)O2]

To a solution of 1-[2-[[(1S)-1-(2,2-difluoro-1,3-benzodioxol-5-yl)ethyl]amino]-4-pyridyl]-3-(trifluoromethyl)-5,6-dihydro-4H-indazol-7-one (1.00 eq, 350 mg, 0.729 mmol) in IPA (5 mL) were added RuCl(p-cymene)[(R,R)-Ts-DPEN] (CAS No.: 192139-92-7) (0.0647 eq, 30 mg, 0.0472 mmol), triethylamine (1.18 eq, 0.12 mL, 0.861 mmol), and formic acid (2.86 eq, 0.080 mL, 2.09 mmol) slowly at 0° C. under N₂. The reaction was stirred at 45° C. for 1 h and concentrated in vacuo. The residue was purified by silica gel column chromatography (0-30% ethyl acetate in heptane) to yield (7R)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-ol (330 mg, 0.684 mmol, 93.9% yield) as a brown solid.

LC-MS (Method 4): Mass: find peak 483.0 [M+H]⁺. At 2.16 min.

### Step 5. Synthesis of 2-((S)-1-(2-(((S)-1-(2,2-difluorobenzo[d][1,3]dioxol-5-yl)ethyl)amino)pyridine-4-yl)-3-(trifluoromethyl)-4,5,6,7-tetrahydro-1H-indazol-7-yl) isoindoline-1,3-dione

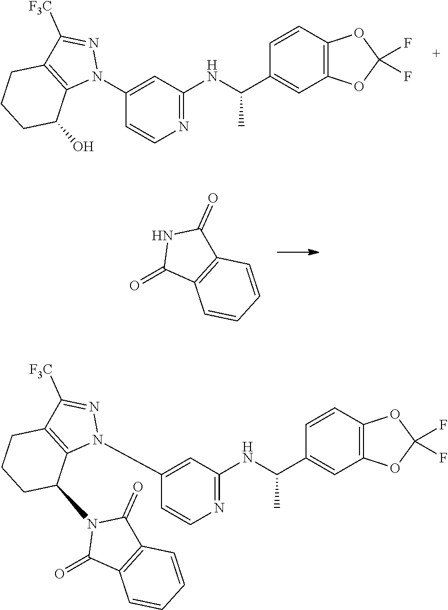 [SMILES: C[C@H](Nc1cc(-n2nc(C(F)(F)F)c3c2[C@H](O)CCC3)ccn1)c1ccc2c(c1)OC(F)(F)O2 ; O=C1NC(=O)c2ccccc21 ; C[C@H](Nc1cc(-n2nc(C(F)(F)F)c3c2[C@@H](N2C(=O)c4ccccc4C2=O)CCC3)ccn1)c1ccc2c(c1)OC(F)(F)O2]

To a solution of (7R)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-ol (1.00 eq, 330 mg, 0.684 mmol) in THF (8 mL) were added triphenylphosphine (2.00 eq, 359 mg, 1.37 mmol) and diethyl azodicarboxylate (1.50 eq, 0.16 mL, 1.03 mmol) at 0° C. under N₂. The reaction was stirred for 2 h, then concentrated in vacuo. The residue was purified by silica gel column chromatography (0-20% ethyl acetate in heptane) to yield 2-[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]isoindoline-1,3-dione (250 mg, 0.380 mmol, 55.6% yield) as a white solid.

LC-MS (Method 4): 612.0 [M+H]⁺. At 2.27 min.

### Step 6. Synthesis of(S)-1-(2-(((S)-1-(2,2-difluorobenzo[d][1,3]dioxol-5-yl)ethyl)amino)pyridine-4-yl)-3-(trifluoromethyl)-4,5,6,7-tetrahydro-1H-indazol-7-amine

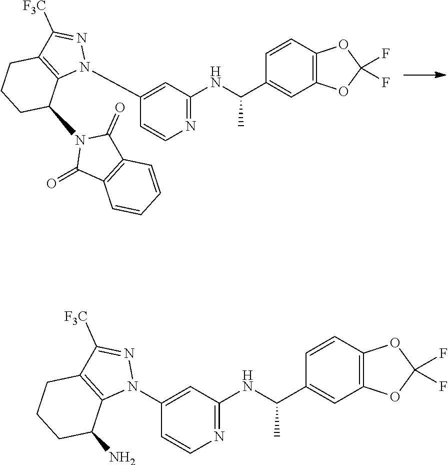 [SMILES: C[C@H](Nc1cc(-n2nc(C(F)(F)F)c3c2[C@@H](N2C(=O)c4ccccc4C2=O)CCC3)ccn1)c1ccc2c(c1)OC(F)(F)O2 ; C[C@H](Nc1cc(-n2nc(C(F)(F)F)c3c2[C@@H](N)CCC3)ccn1)c1ccc2c(c1)OC(F)(F)O2]

To a solution of 2-[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]isoindoline-1,3-dione (1.00 eq, 250 mg, 0.409 mmol) in methanol (2 mL) and THF (2 mL) was added hydrazine hydrate (50.4 eq, 1.0 mL, 20.6 mmol). The reaction was stirred at 80° C. for 18 h, then water (20 mL) was added. The mixture was extracted with EA (20 mL×2). The combined EA solution was washed with brine (20 mL), then concentrated in vacuo. The residue was purified by reverse phase combi-flash (A: water (10 mmol/L NH₄CO₃); B: ACN) to yield (7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-amine (150 mg, 0.312 mmol, 76.2% yield) as a white solid.

LC-MS (Method 4): 482.1 [M+H]⁺. At 2.15 min.

### Step 7. Synthesis of methyl 4-(((S)-1-(2-(((S)-1-(2,2-difluorobenzo[d][1,3]dioxol-5-yl)ethyl)amino)pyridine-4-yl)-3-(trifluoromethyl)-4,5,6,7-tetrahydro-1H-indazol-7-yl)amino) benzoate

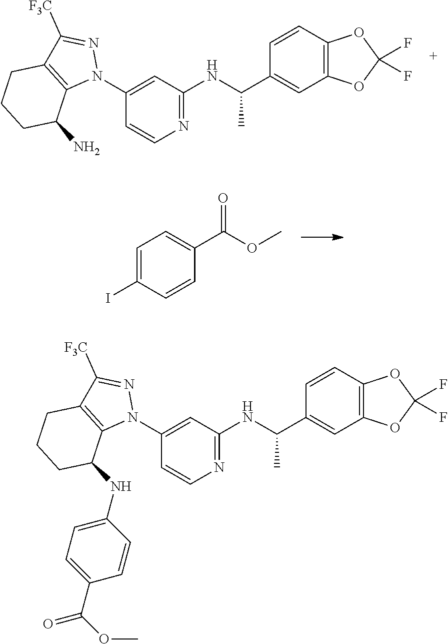 [SMILES: C[C@H](Nc1cc(-n2nc(C(F)(F)F)c3c2[C@@H](N)CCC3)ccn1)c1ccc2c(c1)OC(F)(F)O2 ; COC(=O)c1ccc(I)cc1 ; COC(=O)c1ccc(N[C@H]2CCCc3c(C(F)(F)F)nn(-c4ccnc(N[C@@H](C)c5ccc6c(c5)OC(F)(F)O6)c4)c32)cc1]

To a solution of (7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-amine (1.00 eq, 100 mg, 0.208 mmol) in 1,4-dioxane (2 mL) were added methyl 4-iodobenzoate (1.50 eq, 82 mg, 0.312 mmol) and cesium carbonate (2.00 eq, 135 mg, 0.415 mmol), followed GPhos Pd G3 (CAS No.: 2489525-82-6) (0.0531 eq, 10 mg, 0.0110 mmol) under N₂. The reaction was stirred at 100° C. for 1 h, then water (20 mL) was added. The mixture extracted with EA (20 mL×2). The combined EA solution was washed with brine (20 mL), then concentrated in vacuo. The residue was purified by silica gel column chromatography (0-35% ethyl acetate in heptane) to yield methyl 4-[[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]amino]benzoate (55 mg, 0.0894 mmol, 43.02% yield) as a white solid.

LC-MS (Method 4): Retention time=2.34 min. MS (ESI) m/z 616.0 [M+H]⁺.

### Step 8. Synthesis of Synthesis of 4-[[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]amino]benzoic acid

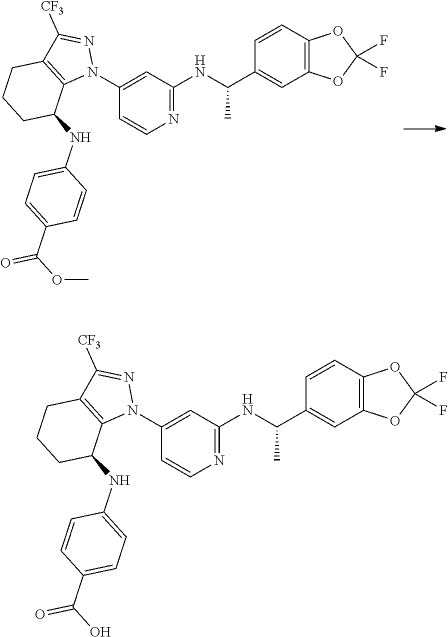 [SMILES: COC(=O)c1ccc(N[C@H]2CCCc3c(C(F)(F)F)nn(-c4ccnc(N[C@@H](C)c5ccc6c(c5)OC(F)(F)O6)c4)c32)cc1 ; C[C@H](Nc1cc(-n2nc(C(F)(F)F)c3c2[C@@H](Nc2ccc(C(=O)O)cc2)CCC3)ccn1)c1ccc2c(c1)OC(F)(F)O2]

To a solution of methyl 4-[[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]amino]benzoate (1.00 eq, 55 mg, 0.0894 mmol) in methanol (1 mL), THF (1 mL) and water (0.5 mL) was added lithium hydroxide (5.00 eq, 11 mg, 0.447 mmol). The reaction was stirred at room temperature for 18 h. The solvent was removed, and the pH was adjusted to ˜3 by addition of citric acid. The solution was diluted with water (20 mL), extracted with EA (20 mL×2). The EA solution was washed with brine (20 mL) and concentrated in vacuo. The residue was purified by prep-HPLC (A: water (10 mmol/L NH₄CO₃); B: ACN) to give 4-[[(7S)-1-[2-[[(1S)-1-(2,2-difluoro-1,3-benzodioxol-5-yl)ethyl]amino]-4-pyridyl]-3-(trifluoromethyl)-4,5,6,7-tetrahydroindazol-7-yl]amino]benzoic acid (28 mg, 0.0457 mmol, 51.2% yield) as a white solid.

LC-MS (Method 4): Retention time=1.75 min. MS (ESI) m/z 602.0 [M+H]⁺.

¹H NMR (400 MHZ, MeOH-d₄) δ:7.88 (dd, J=15.8, 7.2 Hz, 3H), 7.04 (d, J=8.3 Hz, 1H), 6.99 (d, J=1.5 Hz, 1H), 6.93 (dd, J=8.2, 1.6 Hz, 1H), 6.85-6.78 (m, 2H), 6.69 (d, J=8.8 Hz, 2H), 4.93 (m, 1H), 4.46 (d, J=6.9 Hz, 1H), 2.82 (d, J=15.0 Hz, 1H), 2.67-2.47 (m, 1H), 2.13 (d, J=13.6 Hz, 1H), 1.92 (d, J=30.0 Hz, 2H), 1.72 (t, J=13.3 Hz, 1H), 1.30 (d, J=6.9 Hz, 3H).

### Example 94 (P1 and P2)

### 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]amino]bicyclo[1.1.1]pentane-1-carboxylic acid

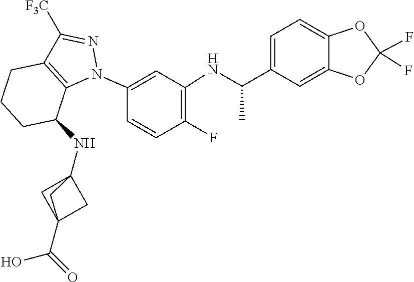 [SMILES: C[C@H](Nc1cc(-n2nc(C(F)(F)F)c3c2[C@@H](NC24CC(C(=O)O)(C2)C4)CCC3)ccc1F)c1ccc2c(c1)OC(F)(F)O2]

### 3-[[(7R)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]amino]bicyclo[1.1.1]pentane-1-carboxylic acid

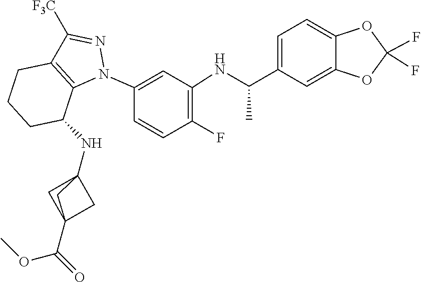 [SMILES: COC(=O)C12CC(N[C@@H]3CCCc4c(C(F)(F)F)nn(-c5ccc(F)c(N[C@@H](C)c6ccc7c(c6)OC(F)(F)O7)c5)c43)(C1)C2]

### Step 1. Synthesis of 1-(3-bromo-4-fluorophenyl)-3-(trifluoromethyl)-1,4,5,6-tetrahydrospiro[indazole-7,2′-[1,3]dioxolane]

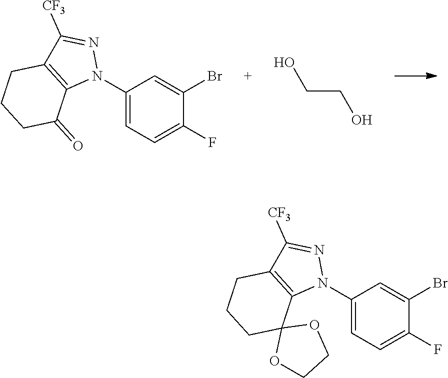

To a solution of 1-(3-bromo-4-fluoro-phenyl)-3-(trifluoromethyl)-5,6-dihydro-4H-indazol-7-one (Intermediate D-3a) (1.00 eq, 1000 mg, 2.65 mmol) in toluene (10 mL) were added ethylene glycol (1.50 eq, 0.22 mL, 3.98 mmol) and p-toluenesulfonic acid (0.100 eq, 46 mg, 0.265 mmol). The reaction was stirred at 115° C. for 4 h. LC-MS showed that the starting material was consumed completely. The reaction mixture was concentrated in vacuo to give a residue, which was purified by flash silica gel column chromatography (0% to 15% ethyl acetate in petroleum ether from) to afford 1′-(3-bromo-4-fluoro-phenyl)-3′-(trifluoromethyl)spiro[1,3-dioxolane-2,7′-5,6-dihydro-4H-indazole] (1100 mg, 2.56 mmol, 96.5% yield) as a yellow solid.

LCMS (Method 2): Retention time=2.18 min. MS (ESI) m/z 421.0, 423.0 [M+H]⁺.

### Step 2. Synthesis of(S)—N-(1-(2,2-difluorobenzo[d][1,3]dioxol-5-yl)ethyl)-2-fluoro-5-(3-(trifluoromethyl)-5,6-dihydrospiro[indazole-7,2′-[1,3]dioxolan]-1 (4H)-yl) aniline

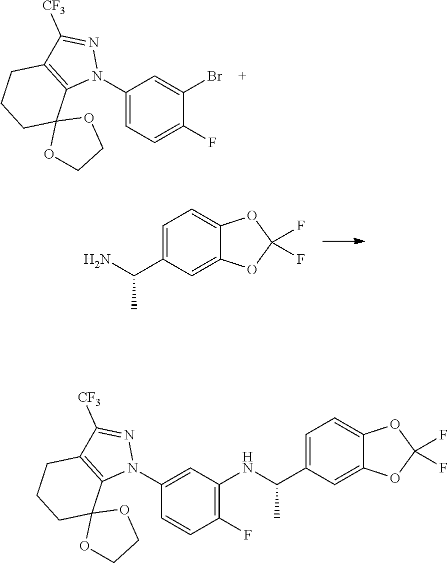 [SMILES: Fc1ccc(-n2nc(C(F)(F)F)c3c2C2(CCC3)OCCO2)cc1Br ; C[C@H](N)c1ccc2c(c1)OC(F)(F)O2 ; C[C@H](Nc1cc(-n2nc(C(F)(F)F)c3c2C2(CCC3)OCCO2)ccc1F)c1ccc2c(c1)OC(F)(F)O2]

To a solution of 1′-(3-bromo-4-fluoro-phenyl)-3′-(trifluoromethyl)spiro[1,3-dioxolane-2,7′-5,6-dihydro-4H-indazole] (1.00 eq, 1100 mg, 2.61 mmol) in 1,4-dioxane (15 mL) were added (1S)-1-(2,2-difluoro-1,3-benzodioxol-5-yl)ethanamine (Intermediate C-6a) (1.20 eq, 630 mg, 3.13 mmol), BrettPhos Pd G3 (0.0500 eq, 118 mg, 0.131 mmol) and cesium carbonate (3.00 eq, 2553 mg, 7.84 mmol) under N₂. The reaction was stirred at 100° C. for 8 h. The reaction mixture was concentrated in vacuo to give a residue which was purified by flash silica gel column chromatography (0% to 15% ethyl acetate in petroleum ether) to obtain N-[(1S)-1-(2,2-difluoro-1,3-benzodioxol-5-yl)ethyl]-2-fluoro-5-[3′-(trifluoromethyl)spiro[1,3-dioxolane-2,7′-5,6-dihydro-4H-indazole]-1′-yl]aniline (800 mg, 1.45 mmol, 55.4% yield) as a white solid.

LCMS (Method 1): Retention time=2.43 min. MS (ESI) m/z 542.0 [M+H]⁺.

### Step 3. Synthesis of(S)-1-(3-((1-(2,2-difluorobenzo[d][1,3]dioxol-5-yl)ethyl)amino)-4-fluorophenyl)-3-(trifluoromethyl)-1,4,5,6-tetrahydro-7H-indazol-7-one

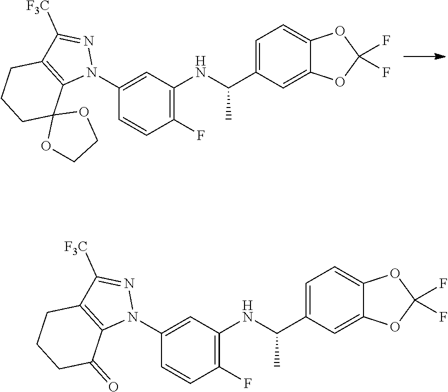 [SMILES: C[C@H](Nc1cc(-n2nc(C(F)(F)F)c3c2C2(CCC3)OCCO2)ccc1F)c1ccc2c(c1)OC(F)(F)O2 ; C[C@H](Nc1cc(-n2nc(C(F)(F)F)c3c2C(=O)CCC3)ccc1F)c1ccc2c(c1)OC(F)(F)O2]

The solution of N-[(1S)-1-(2,2-difluoro-1,3-benzodioxol-5-yl)ethyl]-2-fluoro-5-[3′-(trifluoromethyl)spiro[1,3-dioxolane-2,7′-5,6-dihydro-4H-indazole]-1′-yl]aniline (1.00 eq, 800 mg, 1.48 mmol) in formic acid (176 eq, 10 mL, 261 mmol) was stirred at room temperature for 1 h. The reaction mixture was concentrated in vacuo to give a residue which was purified by flash silica gel column chromatography (0% to 20% ethyl acetate in petroleum ether) to obtain 1-[3-[[(1S)-1-(2,2-difluoro-1,3-benzodioxol-5-yl)ethyl]amino]-4-fluoro-phenyl]-3-(trifluoromethyl)-5,6-dihydro-4H-indazol-7-one (650 mg, 1.28 mmol, 86.7% yield) as a brown oil.

LCMS (Method 2): Retention time=2.35 min. MS (ESI) m/z 496.0 [M−H]⁺.

### Step 4. Synthesis of 1-(3-(((S)-1-(2,2-difluorobenzo[d][1,3]dioxol-5-yl)ethyl)amino)-4-fluorophenyl)-3-(trifluoromethyl)-4,5,6,7-tetrahydro-1H-indazol-7-ol

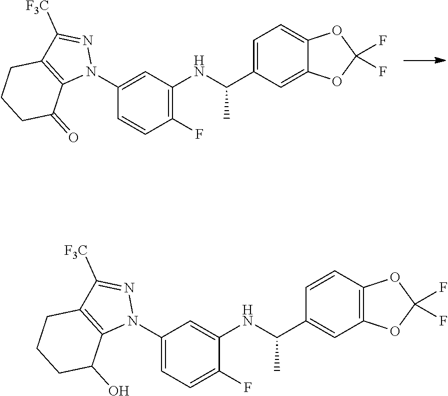 [SMILES: C[C@H](Nc1cc(-n2nc(C(F)(F)F)c3c2C(=O)CCC3)ccc1F)c1ccc2c(c1)OC(F)(F)O2 ; C[C@H](Nc1cc(-n2nc(C(F)(F)F)c3c2C(O)CCC3)ccc1F)c1ccc2c(c1)OC(F)(F)O2]

To a solution of 1-[3-[[(1S)-1-(2,2-difluoro-1,3-benzodioxol-5-yl)ethyl]amino]-4-fluoro-phenyl]-3-(trifluoromethyl)-5,6-dihydro-4H-indazol-7-one (1.00 eq, 650 mg, 1.31 mmol) in methanol (10 mL) was added sodium borohydride (1.20 eq, 59 mg, 1.57 mmol). The reaction was stirred at room temperature for 1 h. The reaction mixture was concentrated in vacuo to give a residue which was purified by flash silica gel column chromatography (0% to 15% ethyl acetate in petroleum ether) to obtain 1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-ol (600 mg, 1.18 mmol, 90.1% yield) as a yellow oil.

LCMS (Method 2): Retention time=2.25 min. MS (ESI) m/z 500.1 [M+H]⁺.

### Step 5. Synthesis of 5-(7-bromo-3-(trifluoromethyl)-4,5,6,7-tetrahydro-1H-indazol-1-yl)-N—((S)-1-(2,2-difluorobenzo[d][1,3]dioxol-5-yl)ethyl)-2-fluoroaniline

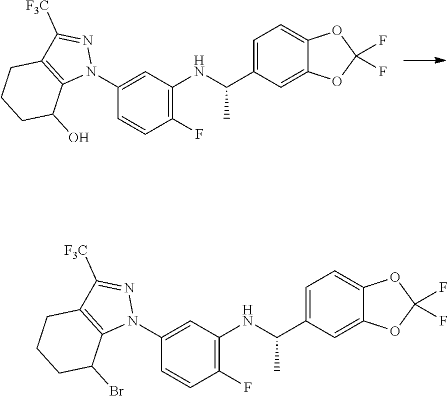 [SMILES: C[C@H](Nc1cc(-n2nc(C(F)(F)F)c3c2C(O)CCC3)ccc1F)c1ccc2c(c1)OC(F)(F)O2 ; C[C@H](Nc1cc(-n2nc(C(F)(F)F)c3c2C(Br)CCC3)ccc1F)c1ccc2c(c1)OC(F)(F)O2]

To a solution of 1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-ol (1.00 eq, 600 mg, 1.20 mmol) in DCM (10 mL) was added phosphorus tribromide (1.20 eq, 0.14 mL, 1.44 mmol). The reaction was stirred at room temperature for 1 h. The reaction mixture was concentrated in vacuo to give a residue, which was purified by flash silica gel column chromatography (0% to 15% ethyl acetate in petroleum ether) to obtain 5-[7-bromo-3-(trifluoromethyl)-4,5,6,7-tetrahydroindazol-1-yl]-N-[(1S)-1-(2,2-difluoro-1,3-benzodioxol-5-yl)ethyl]-2-fluoro-aniline (500 mg, 0.871 mmol, 72.53% yield) as a colorless oil.

LCMS (Method 1): Retention time=2.40 min. MS (ESI) m/z 562.1, 564.1 [M+H]⁺.

### Step 6. Synthesis of methyl 3-(((S)-1-(3-(((S)-1-(2,2-difluorobenzo[d][1,3]dioxol-5-yl)ethyl)amino)-4-fluorophenyl)-3-(trifluoromethyl)-4,5,6,7-tetrahydro-1H-indazol-7-yl)amino)bicyclo[1.1.1]pentane-1-carboxylate and methyl 3-(((R)-1-(3-(((S)-1-(2,2-difluorobenzo[d][1,3]dioxol-5-yl)ethyl)amino)-4-fluorophenyl)-3-(trifluoromethyl)-4,5,6,7-tetrahydro-1H-indazol-7-yl)amino)bicyclo[1.1.1]pentane-1-carboxylate

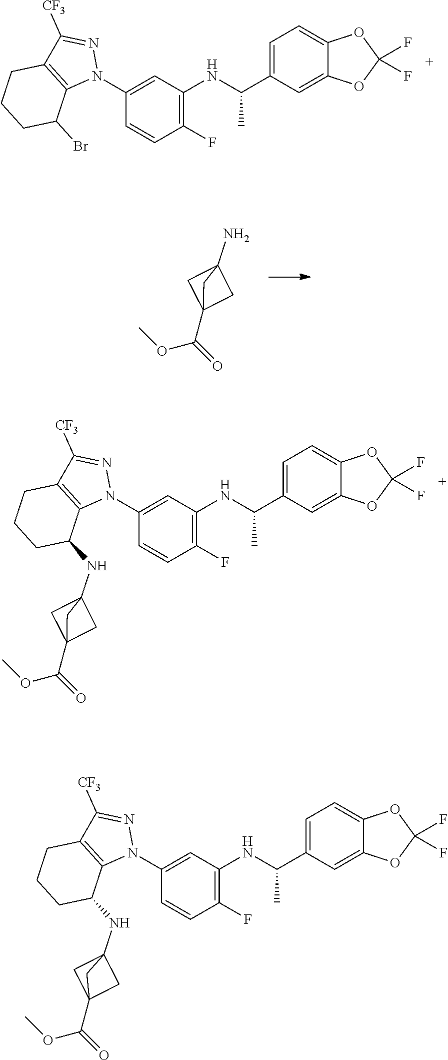

To a solution of 5-[7-bromo-3-(trifluoromethyl)-4,5,6,7-tetrahydroindazol-1-yl]-N-[(1S)-1-(2,2-difluoro-1,3-benzodioxol-5-yl)ethyl]-2-fluoro-aniline (1.00 eq, 100 mg, 0.178 mmol) in acetonitrile (2 mL) were added methyl 3-aminobicyclo[1.1.1]pentane-1-carboxylate (1.20 eq, 30 mg, 0.213 mmol) and potassium carbonate (2.00 eq, 49 mg, 0.356 mmol). The reaction was stirred at 50° C. for 16 h. The reaction mixture was concentrated in vacuo to give a residue which was purified by prep-HPLC (NH₄HCO₃ condition) to afford a racemic mixture which was then separated by SFC to obtain Fraction 1 (25 mg, 0.0402 mmol, 22.5% yield) as a white solid and Fraction 2 (20 mg, 0.0321 mmol, 18.1% yield) as a white solid.

LCMS (Method 2): Retention time=2.44 min. MS (ESI) m/z 623.1 [M+H]⁺.

### Step 7a. Synthesis of 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]amino]bicyclo[1.1.1]pentane-1-carboxylic acid

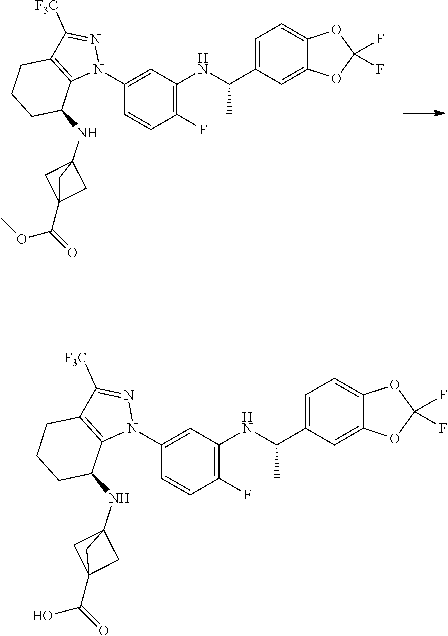 [SMILES: COC(=O)C12CC(N[C@H]3CCCc4c(C(F)(F)F)nn(-c5ccc(F)c(N[C@@H](C)c6ccc7c(c6)OC(F)(F)O7)c5)c43)(C1)C2 ; C[C@H](Nc1cc(-n2nc(C(F)(F)F)c3c2[C@@H](NC24CC(C(=O)O)(C2)C4)CCC3)ccc1F)c1ccc2c(c1)OC(F)(F)O2]

To a solution of Fraction 1 obtained above (1.00 eq, 20 mg, 0.0321 mmol) in methanol (1 mL)/THF (1 mL)/water (0.5 mL) was added lithium hydroxide monohydrate (5.00 eq, 6.7 mg, 0.161 mmol). The reaction was stirred at room temperature for 1 h. The reaction mixture was diluted with water (10 mL), the pH adjusted to ˜5 with citric acid aqueous solution and then extracted with ethyl acetate (10 mL×3). The combined ethyl acetate solution was concentrated to afford a residue which was purified by prep-HPLC (NH₄HCO₃ condition) to obtain 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]amino]bicyclo[1.1.1]pentane-1-carboxylic acid (12 mg, 0.0189 mmol, 58.8% yield) as a white solid.

### P1:

LCMS (Method 2): Retention time=1.75 min. MS (ESI) m/z 609.0 [M+H]⁺.

¹H NMR (400 MHZ, MeOH-d₄) δ:7.26 (d, J=1.5 Hz, 1H), 7.19 (dd, J=8.3, 1.5 Hz, 1H), 7.14-7.04 (m, 2H), 6.79 (ddd, J=8.4, 3.9, 2.6 Hz, 1H), 6.59 (dd, J=7.6, 2.4 Hz, 1H), 4.64 (q, J=6.8 Hz, 1H), 3.99 (d, J=3.8 Hz, 1H), 2.66 (d, J=16.2 Hz, 1H), 2.49 (dd, J=15.5, 5.7 Hz, 1H), 1.87-1.72 (m, 7H), 1.66 (m, 3H), 1.55 (m, 3H).

### Step 7b. Synthesis of 3-[[(7R)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]amino]bicyclo[1.1.1]pentane-1-carboxylic acid

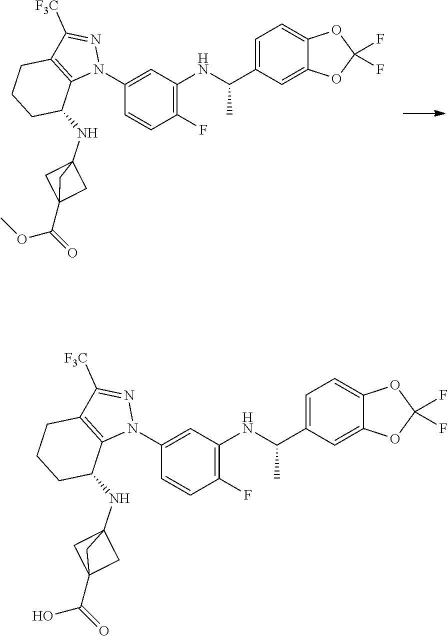 [SMILES: COC(=O)C12CC(N[C@@H]3CCCc4c(C(F)(F)F)nn(-c5ccc(F)c(N[C@@H](C)c6ccc7c(c6)OC(F)(F)O7)c5)c43)(C1)C2 ; C[C@H](Nc1cc(-n2nc(C(F)(F)F)c3c2[C@H](NC24CC(C(=O)O)(C2)C4)CCC3)ccc1F)c1ccc2c(c1)OC(F)(F)O2]

The identical procedures as Step 7a above were applied to obtain P2.

### P2:

LCMS (Method 2): Retention time=1.74 min. MS (ESI) m/z 609.2 [M+H]⁺.

¹H NMR (400 MHZ, MeOH-d₄) δ:7.32 (d, J=1.5 Hz, 1H), 7.25 (dd, J=8.3, 1.5 Hz, 1H), 7.16-7.04 (m, 2H), 6.84-6.73 (m, 1H), 6.61 (dd, J=7.7, 2.4 Hz, 1H), 4.57 (q, J=6.8 Hz, 1H), 3.46 (s, 1H), 2.68 (d, J=13.7 Hz, 1H), 2.55-2.38 (m, 1H), 1.91-1.72 (m, 3H), 1.63 (m, 4H), 1.58-1.51 (m, 6H).

The following examples (Example 95-101) were synthesized similarly to Example 94 (P1 and P2):

| Ex No. | Structure | Name | LCMS/NMR |
|---|---|---|---|
| 95 (P1) | 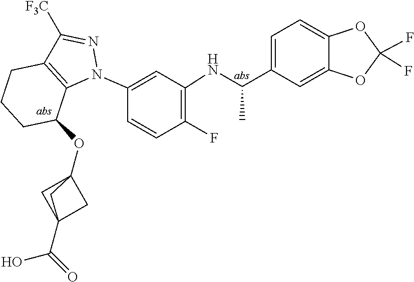 [SMILES: C[C@H](Nc1cc(-n2nc(C(F)(F)F)c3c2[C@@H](OC24CC(C(=O)O)(C2)C4)CCC3)ccc1F)c1ccc2c(c1)OC(F)(F)O2] | 3-[[(7S)-1-[3-[(1S)-1- (2,2-difluoro-1,3- benzodioxol-5- yl)ethyl]amino]-4-fluoro- phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.74 min. MS (ESI) m/z 610.0 [M + H]⁺. ¹H NMR (400 MHZ, MeOH- d4) δ: 7.33 (d, J = 1.5 Hz, 1H), 7.25 (dd, J = 8.3, 1.6 Hz, 1H), 7.12 (dd, J = 11.2, 8.4 Hz, 2H), 6.66 (ddd, J = 8.4, 3.9, 2.6 Hz, 1H), 6.54 (dd, J = 7.6, 2.4 Hz, 1H), 4.56 (q, J = 6.8 Hz, 1H), 4.24 (t, J = 3.1 Hz, 1H), 2.72 (d, J= 16.3 Hz, 1H), 2.54-2.40 (m, 1H), 1.98-1.89 (m, 1H), 1.86-1.75 (m, 5H), 1.65 (m, 4H), 1.55 (d, J = 6.8 Hz, 3H). |
| 95 (P2) | 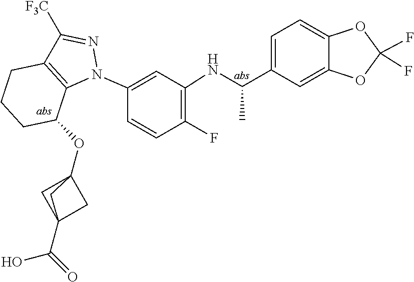 [SMILES: C[C@H](Nc1cc(-n2nc(C(F)(F)F)c3c2[C@H](OC24CC(C(=O)O)(C2)C4)CCC3)ccc1F)c1ccc2c(c1)OC(F)(F)O2] | 3-[[(7R)-1-[3-[(1S)-1- (2,2-difluoro-1,3- benzodioxol-5- yl)ethyl]amino]-4-fluoro- phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.75 min. MS (ESI) m/z 610.0 [M + H]⁺. ¹H NMR (400 MHZ, MeOH- d4) δ: 7.26 (d, J = 1.4 Hz, 1H), 7.19 (dd, J = 8.3, 1.4 Hz, 1H), 7.15-7.04 (m, 2H), 6.71-6.62 (m, 1H), 6.56 (dd, J = 7.6, 2.4 Hz, 1H), 4.72 (s, 1H), 4.64 (q, J = 6.7 Hz, 1H), 2.71 (d, J = 16.2 Hz, 1H), 2.47 (dd, J = 15.7, 8.7 Hz, 1H), 1.99 (t, J = 11.1 Hz, 1H), 1.87-1.73 (m, 9H), 1.55 (d, J = 6.8 Hz, 3H). |
| 96 (P1) | 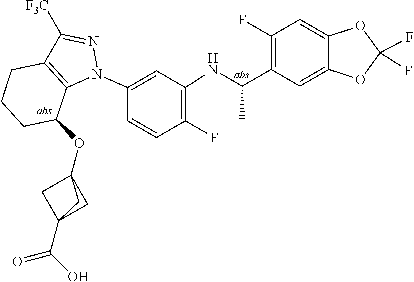 [SMILES: C[C@H](Nc1cc(-n2nc(C(F)(F)F)c3c2[C@@H](OC24CC(C(=O)O)(C2)C4)CCC3)ccc1F)c1cc2c(cc1F)OC(F)(F)O2] | 3-[[(7S)-1-[4-fluoro-3- [[(1S)-1-(2,2,6-trifluoro- 1,3-benzodioxol-5- yl)ethyl]amino]phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.77 min. MS (ESI) m/z 628.0 [M + H]⁺. ¹H NMR (400 MHz, MeOH- d4) δ: 7.33 (d, J = 5.9 Hz, 1H), 7.20-7.03 (m, 2H), 6.71 (ddd, J = 8.4, 3.9, 2.5 Hz, 1H), 6.53 (dd, J = 7.6, 2.4 Hz, 1H), 4.84 (s, 1H), 4.38 (t, J = 3.2 Hz, 1H), 2.73 (d, J = 15.8 Hz, 1H), 2.49 (ddd, J = 16.7, 10.3, 6.5 Hz, 1H), 1.99 (dd, J = 24.1, 12.5 Hz, 1H), 1.92-1.78 (m, 5H), 1.75 (dd, J = 9.3, 1.1 Hz, 3H), 1.71-1.60 (m, 1H), 1.58 (d, J = 6.8 Hz, 3H). |
| 96 (P2) | 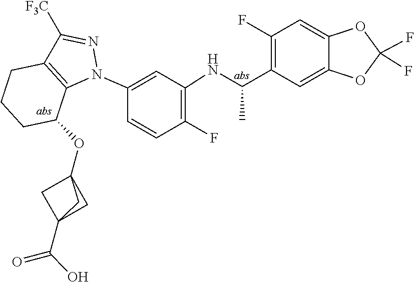 [SMILES: C[C@H](Nc1cc(-n2nc(C(F)(F)F)c3c2[C@H](OC24CC(C(=O)O)(C2)C4)CCC3)ccc1F)c1cc2c(cc1F)OC(F)(F)O2] | 3-[[(7R)-1-[4-fluoro-3- [[(1S)-1-(2,2,6-trifluoro- 1,3-benzodioxol-5- yl)ethyl]amino]phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.77 min. MS (ESI) m/z 628.0 [M + H]⁺. ¹H NMR (400 MHZ, MeOH- d4) δ: 7.27 (d, J = 5.9 Hz, 1H), 7.20-7.01 (m, 2H), 6.72 (ddd, J = 8.4, 3.9, 2.5 Hz, 1H), 6.52 (dd, J = 7.6, 2.4 Hz, 1H), 4.95-4.89 (m, 1H), 4.73 (d, J = 3.4 Hz, 1H), 2.72 (d, J = 16.6 Hz, 1H), 2.48 (dd, J = 16.0, 9.4 Hz, 1H), 2.07-1.93 (m, 1H), 1.93-1.69 (m, 9H), 1.57 (d, J = 6.7 Hz, 3H). |
| 97 (P1) | 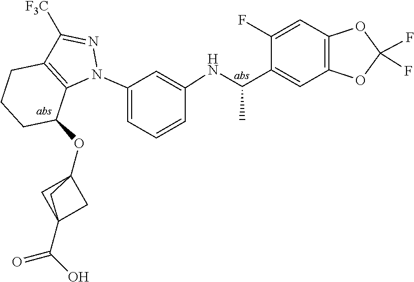 [SMILES: C[C@H](Nc1cccc(-n2nc(C(F)(F)F)c3c2[C@@H](OC24CC(C(=O)O)(C2)C4)CCC3)c1)c1cc2c(cc1F)OC(F)(F)O2] | 3-[[(7S)-1-[3-[(1S)-1- (2,2,6-trifluoro-1,3- benzodioxol-5- yl)ethyl]amino]phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.75 min. MS (ESI) m/z 610.2 [M + H]⁺. ¹H NMR (400 MHZ, MeOH- d4-d4) 8 7.21 (dd, J = 14.8, 6.9 Hz, 2H), 7.09 (d, J = 9.1 Hz, 1H), 6.70 (d, J = 7.3 Hz, 1H), 6.64 (d, J = 7.6 Hz, 2H), 4.85-4.81 (m, 1H), 4.74 (t, J= 3.2 Hz, 1H), 2.74 (d, J = 15.8 Hz, 1H), 2.56-2.45 (m, 1H), 2.06-1.99 (m, 1H), 1.95 (dd, J = 9.3, 1.2 Hz, 3H), 1.89-1.73 (m, 6H), 1.52 (d, J = 6.7 Hz, 3H). |
| 97 (P2) | 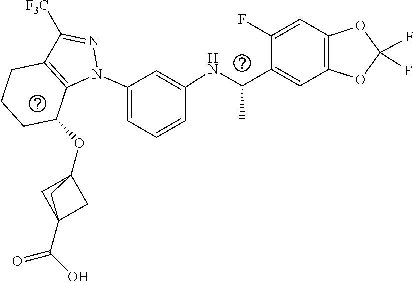 [SMILES: C[C@H](Nc1cccc(-n2nc(C(F)(F)F)c3c2[C@H](OC24CC(C(=O)O)(C2)C4)CCC3)c1)c1cc2c(cc1F)OC(F)(F)O2] | 3-[[(7R)-1-[3-[(1S)-1- (2,2,6-trifluoro-1,3- benzodioxol-5- yl)ethyl]amino]phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.74 min. MS (ESI) m/z 610.0 [M + H]⁺. 'H NMR (400 MHZ, MeOH- d4-d4) 8 7.26 (d, J = 5.9 Hz, 1H), 7.21 (t, J = 8.0 Hz, 1H), 7.10 (d, J = 9.1 Hz, 1H), 6.73- 6.63 (m, 2H), 6.58 (t, J = 2.1 Hz, 1H), 4.85-4.77 (m, 1H), 4.56 (t, J = 3.2 Hz, 1H), 2.75 (d, J = 16.0 Hz, 1H), 2.59-2.44 (m, 1H), 2.01 (d, J = 11.8 Hz, 1H), 1.95-1.77 (m, 8H), 1.76-1.65 (m, 1H), 1.52 (d, J = 6.7 Hz, 3H). |
| 98 (P1) | 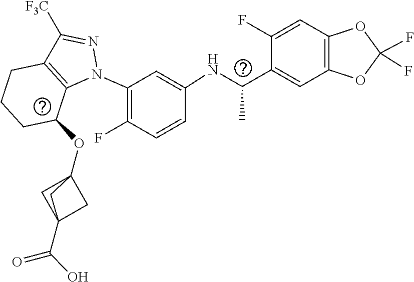 [SMILES: C[C@H](Nc1ccc(F)c(-n2nc(C(F)(F)F)c3c2[C@@H](OC24CC(C(=O)O)(C2)C4)CCC3)c1)c1cc2c(cc1F)OC(F)(F)O2] | 3-[[(7S)-1-[2-fluoro-5- [[(1S)-1-(2,2,6-trifluoro- 1,3-benzodioxol-5- yl)ethyl]amino]phenyl]-3- (trifluoromethyl)-4,5,6,7- tetrahydroindazol-7- yl[oxy]bicyclo[1.1.1] pentane-1-carboxylic acid | LCMS (Method 2): Retention time = 1.74 min. MS (ESI) m/z 628.0 [M + H]⁺. ¹H NMR (400 MHZ, MeOH- d4) δ: 7.24 (d, J = 5.9 Hz, 1H), 7.15-6.99 (m, 2H), 6.75-6.62 (m, 1H), 6.55 (dd, J = 6.0, 2.9 Hz, 1H), 4.84-4.75 (m, 1H), 4.61 (d, J = 3.6 Hz, 1H), 2.72 (d, J = 16.0 Hz, 1H), 2.61-2.44 (m, 1H), 1.94-1.79 (m, 7H), 1.71 (dd, J = 9.3, 1.3 Hz, 3H), 1.51 (d, J = 6.7 Hz, 3H). |
| 98 (P2) | 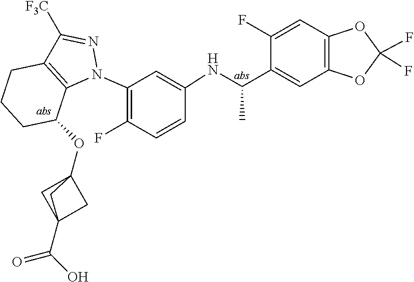 [SMILES: C[C@H](Nc1ccc(F)c(-n2nc(C(F)(F)F)c3c2[C@H](OC24CC(C(=O)O)(C2)C4)CCC3)c1)c1cc2c(cc1F)OC(F)(F)O2] | 3-[[(7R)-1-[2-fluoro-5- [[(1S)-1-(2,2,6-trifluoro- 1,3-benzodioxol-5- yl)ethyl]amino]phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.73 min. MS (ESI) m/z 628.0 [M + H]⁺. ¹H NMR (400 MHZ, MeOH- d4) δ: 7.24 (d, J = 5.9 Hz, 1H), 7.15-6.99 (m, 2H), 6.75-6.62 (m, 1H), 6.55 (dd, J = 6.0, 2.9 Hz, 1H), 4.84-4.75 (m, 1H), 4.61 (d, J = 3.6 Hz, 1H), 2.72 (d, J = 16.0 Hz, 1H), 2.61-2.44 (m, 1H), 1.94-1.79 (m, 7H), 1.71 (dd, J = 9.3, 1.3 Hz, 3H), 1.51 (d, J = 6.7 Hz, 3H). |
| 99 (P1) | 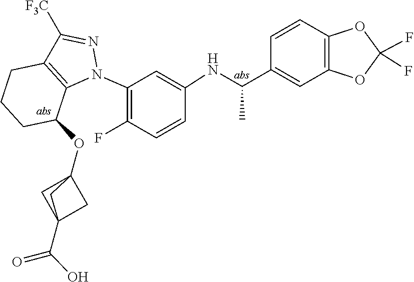 [SMILES: C[C@H](Nc1ccc(F)c(-n2nc(C(F)(F)F)c3c2[C@@H](OC24CC(C(=O)O)(C2)C4)CCC3)c1)c1ccc2c(c1)OC(F)(F)O2] | 3-[[(7S)-1-[5-[(1S)-1- (2,2-difluoro-1,3- benzodioxol-5- yl)ethyl]amino]-2-fluoro- phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.73 min. MS (ESI) m/z 610.0 [M + H]⁺. ¹H NMR (400 MHZ, MeOH- d4) δ: 7.23 (d, J = 1.4 Hz, 1H), 7.19 (dd, J = 8.3, 1.5 Hz, 1H), 7.09 (d, J = 8.2 Hz, 1H), 7.02 (t, J = 9.4 Hz, 1H), 6.68-6.62 (m, 1H), 6.57 (dd, J = 6.1, 2.9 Hz, 1H), 4.62 (d, J = 3.7 Hz, 1H), 4.52 (q, J = 6.7 Hz, 1H), 2.72 (d, J= 16.5 Hz, 1H), 2.60-2.41 (m, 1H), 1.96-1.78 (m, 7H), 1.71 (dd, J = 9.3, 1.1 Hz, 3H), 1.49 (d, J = 6.7 Hz, 3H). |
| 99 (P2) | 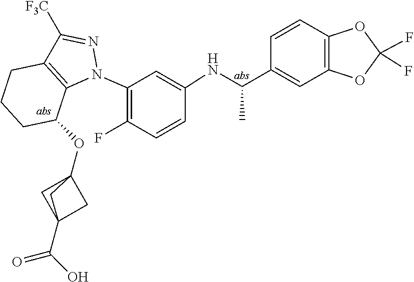 [SMILES: C[C@H](Nc1ccc(F)c(-n2nc(C(F)(F)F)c3c2[C@H](OC24CC(C(=O)O)(C2)C4)CCC3)c1)c1ccc2c(c1)OC(F)(F)O2] | 3-[[(7R)-1-[5-[[(1S)-1- (2,2-difluoro-1,3- benzodioxol-5- yl)ethyl]amino]-2-fluoro- phenyl]-3- (trifluoromethyl)-4,5,6,7- tetrahydroindazol-7- yl]oxy]bicyclo[1.1.1] pentane-1-carboxylic acid | LCMS (Method 2): Retention time = 1.72 min. MS (ESI) m/z 610.0 [M + H]⁺. ¹H NMR (400 MHZ, MeOH- d4) δ: 7.26 (d, J = 1.5 Hz, 1H), 7.21 (dd, J = 8.3, 1.6 Hz, 1H), 7.11 (d, J = 8.2 Hz, 1H), 7.03 (t, J = 9.3 Hz, 1H), 6.72-6.64 (m, 1H), 6.52 (dd, J = 6.0, 2.9 Hz, 1H), 4.47 (q, J = 6.7 Hz, 1H), 4.37 (s, 1H), 2.77-2.68 (m, 1H), 2.55-2.44 (m, 1H), 1.95- 1.87 (m, 1H), 1.87-1.75 (m, 6H), 1.67 (d, J = 9.2 Hz, 3H), 1.49 (d, J = 6.7 Hz, 3H). |
| 100 (P1) | 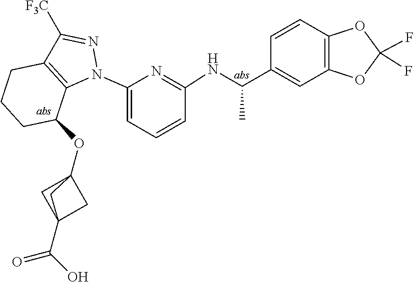 [SMILES: C[C@H](Nc1cccc(-n2nc(C(F)(F)F)c3c2[C@@H](OC24CC(C(=O)O)(C2)C4)CCC3)n1)c1ccc2c(c1)OC(F)(F)O2] | 3-[[(7S)-1-[6-[[(1S)-1- (2,2-difluoro-1,3- benzodioxol-5- yl)ethyl]amino]-2- pyridyl]-3- (trifluoromethyl)-4,5,6,7- tetrahydroindazol-7- yl]oxy]bicyclo[1.1.1] pentane-1-carboxylic acid | LCMS (Method 2): Retention time = 1.72 min. MS (ESI) m/z 593.1 [M + H]⁺. ¹H NMR (400 MHZ, MeOH-d4) δ: 7.63-7.51 (m, 1H), 7.30 (d, J = 1.4 Hz, 1H), 7.24 (dd, J = 8.3, 1.4 Hz, 1H), 7.15 (d, J = 8.2 Hz, 1H), 6.67 (d, J = 7.4 Hz, 1H), 6.57 (d, J = 8.3 Hz, 1H), 4.98-4.89 (m, 2H), 2.73 (d, J = 15.6 Hz, 1H), 2.60-2.45 (m, 1H), 1.91- 1.73 (m, 4H), 1.66 (d, J = 9.1 Hz, 3H), 1.50 (dd, J = 15.1, 8.1 Hz, 6H). |
| 100 (P2) | 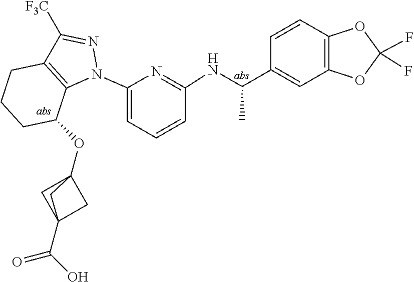 [SMILES: C[C@H](Nc1cccc(-n2nc(C(F)(F)F)c3c2[C@H](OC24CC(C(=O)O)(C2)C4)CCC3)n1)c1ccc2c(c1)OC(F)(F)O2] | 3-[[(7R)-1-[6-[[(1S)-1- (2,2-difluoro-1,3- benzodioxol-5- yl)ethyl]amino]-2- pyridyl]-3- (trifluoromethyl)-4,5,6,7- tetrahydroindazol-7- yl]oxy]bicyclo[1.1.1] pentane-1-carboxylic acid | LCMS (Method 2): Retention time = 1.71 min. MS (ESI) m/z 593.1 [M + H]⁺. ¹H NMR (400 MHZ, MeOH- d4) δ: 7.61-7.51 (m, 1H), 7.22 (d, J = 1.4 Hz, 1H), 7.18 (dd, J = 8.3, 1.5 Hz, 1H), 7.05 (d, J = 8.3 Hz, 1H), 6.73 (d, J = 7.5 Hz, 1H), 6.48 (d, J= 8.3 Hz, 1H), 5.59 (d, J = 3.2 Hz, 1H), 4.98-4.91 (m, 2H), 2.73 (d, J = 16.0 Hz, 1H), 2.60-2.46 (m, 1H), 2.14-2.01 (m, 4H), 2.00- 1.78 (m, 6H), 1.54 (d, J = 6.9 Hz, 3H). |
| 101 (P1) | 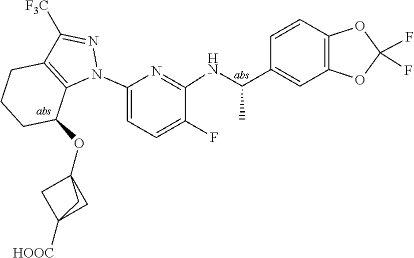 [SMILES: C[C@H](Nc1nc(-n2nc(C(F)(F)F)c3c2[C@@H](OC24CC(C(=O)O)(C2)C4)CCC3)ccc1F)c1ccc2c(c1)OC(F)(F)O2] | 3-[[(7S)-1-[6-[(1S)-1- (2,2-difluoro-1,3- benzodioxol-5- yl)ethyl]amino]-5-fluoro- 2-pyridyl]-3- (trifluoromethyl)-4,5,6,7- tetrahydroindazol-7- yl]oxy]bicyclo[1.1.1] pentane-1-carboxylic acid | LCMS (Method 4): Retention time = 1.82 min. MS (ESI) m/z 611.0 [M + H]⁺. ¹H NMR (400 MHZ, MeOH- d4) δ: 7.45 (dd, J = 10.5, 8.1 Hz, 1H), 7.34 (d, J = 1.5 Hz, 1H), 7.29 (dd, J = 8.3, 1.6 Hz, 1H), 7.23-7.12 (m, 1H), 6.65 (dd, J = 8.1, 2.6 Hz, 1H), 5.04 (q, J = 7.0 Hz, 1H), 4.44 (d, J = 2.9 Hz, 1H), 2.72 (d, J = 16.2 Hz, 1H), 2.58- 2.40 (m, 1H), 1.88-1.74 (m, 3H), 1.74-1.59 (m, 1H), 1.59-1.44 (m, 6H), 1.42- 1.28 (m, 3H). |
| 101 (P2) | 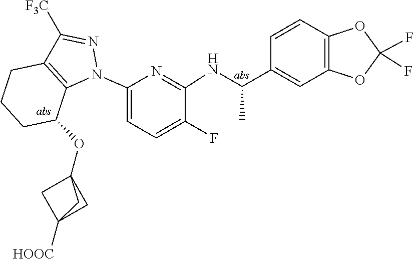 [SMILES: C[C@H](Nc1nc(-n2nc(C(F)(F)F)c3c2[C@H](OC24CC(C(=O)O)(C2)C4)CCC3)ccc1F)c1ccc2c(c1)OC(F)(F)O2] | 3-[[(7R)-1-[6-[(1S)-1- (2,2-difluoro-1,3- benzodioxol-5- yl)ethyl]amino]-5-fluoro- 2-pyridyl]-3- (trifluoromethyl)-4,5,6,7- tetrahydroindazol-7- yl]oxy]bicyclo[1.1.1] pentane-1-carboxylic acid | LCMS (Method 4): Retention time = 1.82 min. MS (ESI) m/z 611.1 [M + H]⁺. ¹H NMR (400 MHZ, MeOH- d4) δ: 7.42 (dd, J = 10.4, 8.2 Hz, 1H), 7.21 (s, 1H), 7.14 (d, J = 8.3 Hz, 1H), 7.01 (d, J= 8.3 Hz, 1H), 6.69 (dd, J = 8.2, 2.5 Hz, 1H), 5.44 (d, J = 3.6 Hz, 1H), 5.23 (q, J = 7.0 Hz, 1H), 2.72 (d, J = 16.6 Hz, 1H), 2.62-2.42 (m, 1H), 2.09 (d, J = 12.1 Hz, 1H), 2.04-1.74 (m, 9H), 1.60 (d, J = 7.1 Hz, 3H). |

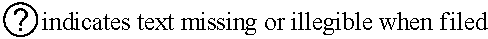

### Example 102 (P1 and P2)

### 3-[3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one

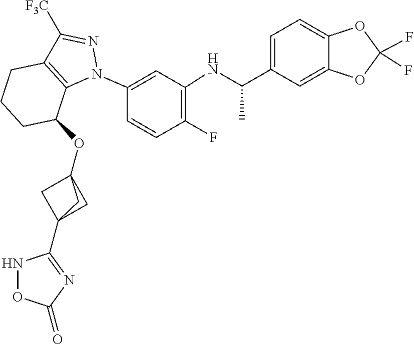 [SMILES: C[C@H](Nc1cc(-n2nc(C(F)(F)F)c3c2[C@@H](OC24CC(c5nc(=O)o[nH]5)(C2)C4)CCC3)ccc1F)c1ccc2c(c1)OC(F)(F)O2]

and

### 3-[3-[[(7R)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one

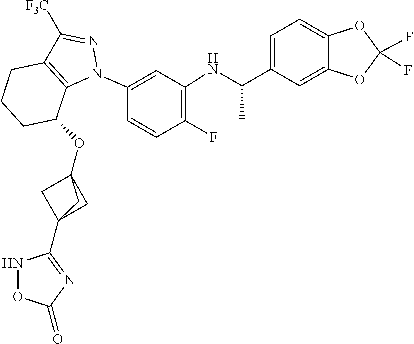 [SMILES: C[C@H](Nc1cc(-n2nc(C(F)(F)F)c3c2[C@H](OC24CC(c5nc(=O)o[nH]5)(C2)C4)CCC3)ccc1F)c1ccc2c(c1)OC(F)(F)O2]

### Step 1. 3-(((S)-1-(3-(((S)-1-(2,2-difluorobenzo[d][1,3]dioxol-5-yl)ethyl)amino)-4-fluorophenyl)-3-(trifluoromethyl)-4,5,6,7-tetrahydro-1H-indazol-7-yl)oxy)bicyclo[1.1.1]pentane-1-carboxamide

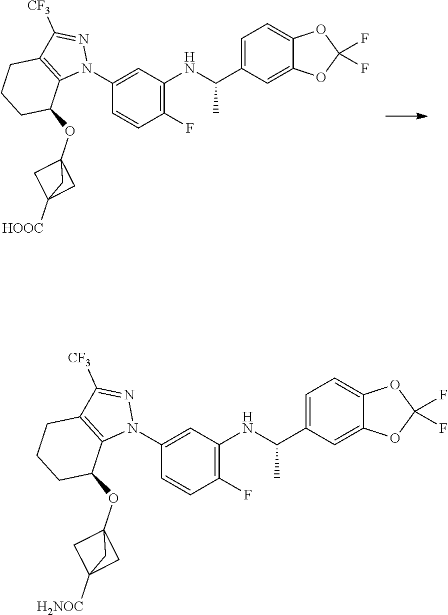 [SMILES: C[C@H](Nc1cc(-n2nc(C(F)(F)F)c3c2[C@@H](OC24CC(C(=O)O)(C2)C4)CCC3)ccc1F)c1ccc2c(c1)OC(F)(F)O2 ; C[C@H](Nc1cc(-n2nc(C(F)(F)F)c3c2[C@@H](OC24CC(C(N)=O)(C2)C4)CCC3)ccc1F)c1ccc2c(c1)OC(F)(F)O2]

To a solution of 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]bicyclo[1.1.1]pentane-1-carboxylic acid (Example 95 (P1)) (1.00 eq, 130 mg, 0.213 mmol) in DMF (3 mL) was added ammonium chloride (1.20 eq, 14 mg, 0.256 mmol), HATU (1.20 eq, 62 mg, 0.256 mmol), and N,N-diisopropylethylamine (5.00 eq, 0.19 mL, 1.07 mmol). The reaction was stirred at rt for 1 h. The mixture was concentrated and the crude was then purified by flash silica gel column chromatography (0-100% EtOAc in PE gradient over 20 min) to obtain 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]bicyclo[1.1.1]pentane-1-carboxamide (120 mg, 0.197 mmol, 92.4% yield) as a yellow solid LCMS (Method 1): Retention time=2.12 min. MS (ESI) m/z 609 [M+H]⁺.

### Step 2. Synthesis of 3-(((S)-1-(3-(((S)-1-(2,2-difluorobenzo[d][1,3]dioxol-5-yl)ethyl)amino)-4-fluorophenyl)-3-(trifluoromethyl)-4,5,6,7-tetrahydro-1H-indazol-7-yl)oxy)bicyclo[1.1.1]pentane-1-carbonitrile

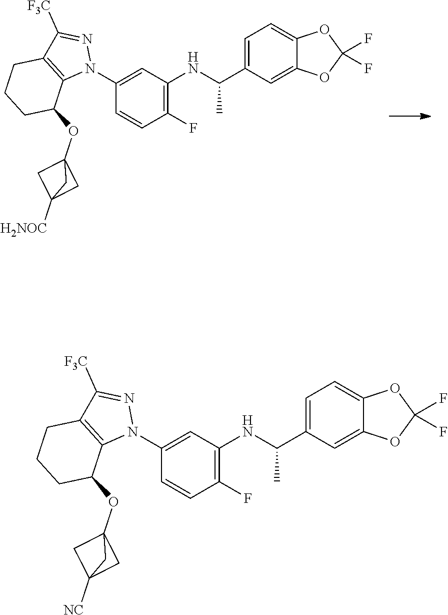 [SMILES: C[C@H](Nc1cc(-n2nc(C(F)(F)F)c3c2[C@@H](OC24CC(C(N)=O)(C2)C4)CCC3)ccc1F)c1ccc2c(c1)OC(F)(F)O2 ; C[C@H](Nc1cc(-n2nc(C(F)(F)F)c3c2[C@@H](OC24CC(C#N)(C2)C4)CCC3)ccc1F)c1ccc2c(c1)OC(F)(F)O2]

To the solution of 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]bicyclo[1.1.1]pentane-1-carboxamide (1.00 eq, 120 mg, 0.197 mmol) in DCM (5 mL) was added N,N-diethyl-N-[[(methoxycarbonyl)amino]sulfonyl]ethanaminium inner salt (5.00 eq, 235 mg, 0.986 mmol). The reaction was stirred at rt for 1 h. The mixture was concentrated and the crude was then purified by flash silica gel column chromatography (0-10% EtOAc in PE gradient over 20 min) to get 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]bicyclo[1.1.1]pentane-1-carbonitrile (100 mg, 0.169 mmol, 85.8% yield) as a white solid.

LCMS (Method 2): Retention time=2.26 min. MS (ESI) m/z 591 [M+H]⁺.

### Step 3. Synthesis of 3-(((S)-1-(3-(((S)-1-(2,2-difluorobenzo[d][1,3]dioxol-5-yl)ethyl)amino)-4-fluorophenyl)-3-(trifluoromethyl)-4,5,6,7-tetrahydro-1H-indazol-7-yl)oxy)-N-hydroxybicyclo[1.1.1]pentane-1-carboximidamide

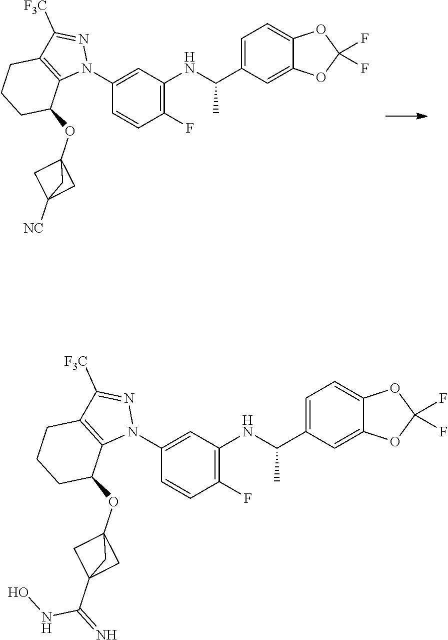 [SMILES: C[C@H](Nc1cc(-n2nc(C(F)(F)F)c3c2[C@@H](OC24CC(C#N)(C2)C4)CCC3)ccc1F)c1ccc2c(c1)OC(F)(F)O2 ; C[C@H](Nc1cc(-n2nc(C(F)(F)F)c3c2[C@@H](OC24CC(C(=N)NO)(C2)C4)CCC3)ccc1F)c1ccc2c(c1)OC(F)(F)O2]

To the solution of 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]bicyclo[1.1.1]pentane-1-carbonitrile (1.00 eq, 100 mg, 0.169 mmol) in ethanol (3 mL) was added hydroxylamine (1.00 eq, 3.0 mL, 0.169 mmol). The reaction was stirred at rt for 2 h. The mixture was concentrated and the crude was then purified by flash silica gel column chromatography (0-100% EtOAc in PE gradient for 20 min) to get 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]-N-hydroxy-bicyclo[1.1.1]pentane-1-carboxamidine (90 mg, 0.144 mmol, 85.2% yield) as a white solid.

LCMS (Method 1): Retention time=2.01 min. MS (ESI) m/z 624.2 [M+H]⁺.

### Step 4. Synthesis of 3-[3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one and 3-[3-[[(7R)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one

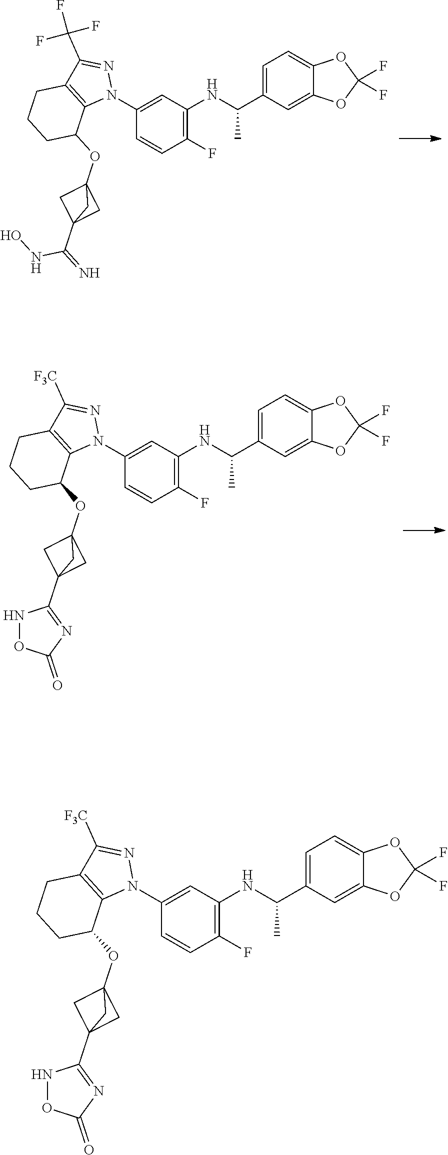 [SMILES: C[C@H](Nc1cc(-n2nc(C(F)(F)F)c3c2C(OC24CC(C(=N)NO)(C2)C4)CCC3)ccc1F)c1ccc2c(c1)OC(F)(F)O2 ; C[C@H](Nc1cc(-n2nc(C(F)(F)F)c3c2[C@@H](OC24CC(c5nc(=O)o[nH]5)(C2)C4)CCC3)ccc1F)c1ccc2c(c1)OC(F)(F)O2 ; C[C@H](Nc1cc(-n2nc(C(F)(F)F)c3c2[C@H](OC24CC(c5nc(=O)o[nH]5)(C2)C4)CCC3)ccc1F)c1ccc2c(c1)OC(F)(F)O2]

To a solution of 3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]-N-hydroxy-bicyclo[1.1.1]pentane-1-carboxamidine (1.00 eq, 60 mg, 0.0962 mmol) in ethyl acetate (5 mL) was added N,N′-carbonyldiimidazole (5.00 eq, 78 mg, 0.481 mmol). The reaction was stirred at 80° C. overnight under Ar. After cooling to room temperature, the mixture was extracted with EA (20 mL×3). The combined organic solutions were concentrated under vacuum. The residue was purified by reverse-phase flash chromatography (C18) (0.1% NH₄HCO₃ in water, 10-100% MeCN) to give the desired racemic product (20 mg) as a white solid. The product was then separated by chiral-HPLC to give 3-[3-[[(7S)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one (P1) (11 mg, 0.0163 mmol, 17.0% yield) as a white solid and 3-[3-[[(7R)-1-[3-[[(1S)-1-(2,2-difluoro-1,3-benzodioxol-5-yl)ethyl]amino]-4-fluoro-phenyl]-3-(trifluoromethyl)-4,5,6,7-tetrahydroindazol-7-yl]oxy]-1-bicyclo[1.1.1]pentanyl]-2H-1,2,4-oxadiazol-5-one (P2) (4.3 mg, 0.00662 mmol, 6.9% yield) as a white solid.

### P1:

LCMS (Method 1): Retention time=1.81 min. MS (ESI) m/z 650 [M+H]⁺.

¹H NMR (400 MHZ, MeOH-d₄) δ:7.33 (d, J=1.4 Hz, 1H), 7.28-7.22 (m, 1H), 7.18-7.09 (m, 2H), 6.76-6.61 (m, 1H), 6.56 (dd, J=7.6, 2.4 Hz, 1H), 4.57 (q, J=6.6 Hz, 1H), 4.31 (t, J=3.2 Hz, 1H), 2.73 (d, J=16.5 Hz, 1H), 2.56-2.35 (m, 1H), 2.02-1.86 (m, 4H), 1.86-1.59 (m, 6H), 1.55 (d, J=6.8 Hz, 3H).

### P2:

LCMS (Method 1): Retention time=1.81 min. MS (ESI) m/z 650 [M+H]⁺.

¹H NMR (400 MHZ, MeOH-d₄) δ:7.26 (d, J=1.5 Hz, 1H), 7.19 (dd, J=8.3, 1.6 Hz, 1H), 7.16-7.06 (m, 2H), 6.77-6.63 (m, 1H), 6.56 (dd, J=7.6, 2.4 Hz, 1H), 4.76 (s, 1H), 4.64 (dd, J=13.6, 6.8 Hz, 1H), 2.72 (d, J=15.9 Hz, 1H), 2.50 (d, J=9.7 Hz, 1H), 2.09-1.90 (m, 4H), 1.90-1.71 (m, 6H), 1.52 (t, J=13.8 Hz, 3H).

### Example 103 (P1 and P2)

### 3-[3-[[(7S)-1-[4-fluoro-3-[[(1S)-1-(2,2,6-trifluoro-1,3-benzodioxol-5-yl)ethyl]amino]phenyl]-3-(trifluoromethyl)-4,5,6,7-tetrahydroindazol-7-yl]oxy]-1-bicyclo[1.1.1]pentanyl]-2H-1,2,4-oxadiazol-5-one

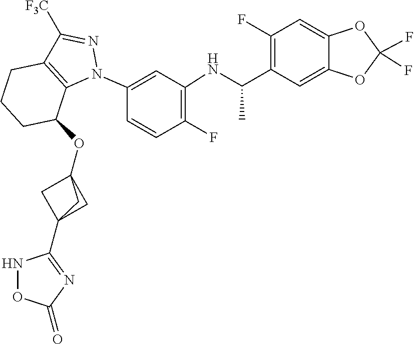 [SMILES: C[C@H](Nc1cc(-n2nc(C(F)(F)F)c3c2[C@@H](OC24CC(c5nc(=O)o[nH]5)(C2)C4)CCC3)ccc1F)c1cc2c(cc1F)OC(F)(F)O2]

and

### 3-[3-[[(7R)-1-[4-fluoro-3-[[(1S)-1-(2,2,6-trifluoro-1,3-benzodioxol-5-yl)ethyl]amino]phenyl]-3-(trifluoromethyl)-4,5,6,7-tetrahydroindazol-7-yl]oxy]-1-bicyclo[1.1.1]pentanyl]-2H-1,2,4-oxadiazol-5-one

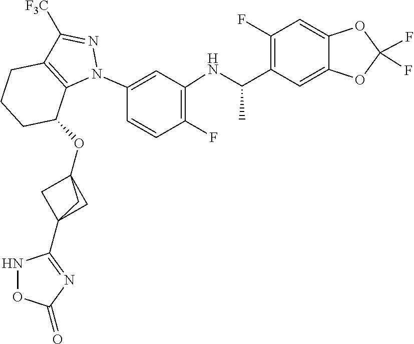 [SMILES: C[C@H](Nc1cc(-n2nc(C(F)(F)F)c3c2[C@H](OC24CC(c5nc(=O)o[nH]5)(C2)C4)CCC3)ccc1F)c1cc2c(cc1F)OC(F)(F)O2]

Example 103 (P1 and P2) were synthesized similarly to Example 102 (P1 and P2) P1:

LCMS (Method 2): Retention time=1.74 min. MS (ESI) m/z 668.3 [M+H]⁺.

¹H NMR (400 MHZ, MeOH-d₄) δ:7.31 (t, J=10.8 Hz, 1H), 7.20-7.04 (m, 2H), 6.73 (ddd, J=8.5, 4.0, 2.5 Hz, 1H), 6.54 (dd, J=7.6, 2.5 Hz, 1H), 4.86 (d, J=6.9 Hz, 1H), 4.45 (t, J=3.2 Hz, 1H), 2.74 (d, J=16.4 Hz, 1H), 2.50 (ddd, J=16.2, 10.0, 6.1 Hz, 1H), 2.09-1.94 (m, 4H), 1.94-1.63 (m, 6H), 1.57 (d, J=6.8 Hz, 3H).

P2:

LCMS (Method 2): Retention time=1.74 min. MS (ESI) m/z 668.3 [M+H]⁺.

¹H NMR (400 MHZ, MeOH-d₄) δ:7.29 (dd, J=16.8, 5.8 Hz, 1H), 7.14 (dd, J=11.3, 8.5 Hz, 1H), 7.07 (d, J=9.1 Hz, 1H), 6.79-6.69 (m, 1H), 6.52 (dd, J=7.6, 2.4 Hz, 1H), 4.88-4.82 (m, 1H), 4.76 (d, J=3.5 Hz, 1H), 2.72 (d, J=15.9 Hz, 1H), 2.49 (dd, J=16.0, 9.5 Hz, 1H), 2.12-2.01 (m, 1H), 2.01-1.91 (m, 3H), 1.91-1.71 (m, 6H), 1.56 (d, J=6.7 Hz, 3H).

### Example 104

### 4-[[(7S)-3-cyano-1-[3-[(1S)-1-(2,2-difluoro-1,3-benzodioxol-5-yl)ethoxy]-4-fluoro-phenyl]-4,5,6,7-tetrahydroindazol-7-yl]oxy]benzoic acid

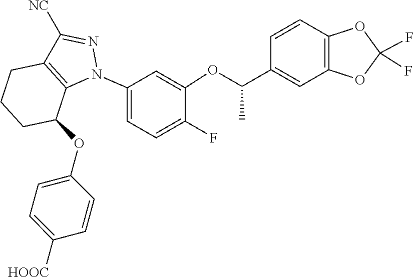 [SMILES: C[C@H](Oc1cc(-n2nc(C#N)c3c2[C@@H](Oc2ccc(C(=O)O)cc2)CCC3)ccc1F)c1ccc2c(c1)OC(F)(F)O2]

### Step 1. Synthesis of(S)-1-(3-(1-(2,2-difluorobenzo[d][1,3]dioxol-5-yl)ethoxy)-4-fluorophenyl)-7-oxo-4,5,6,7-tetrahydro-1H-indazole-3-carbonitrile

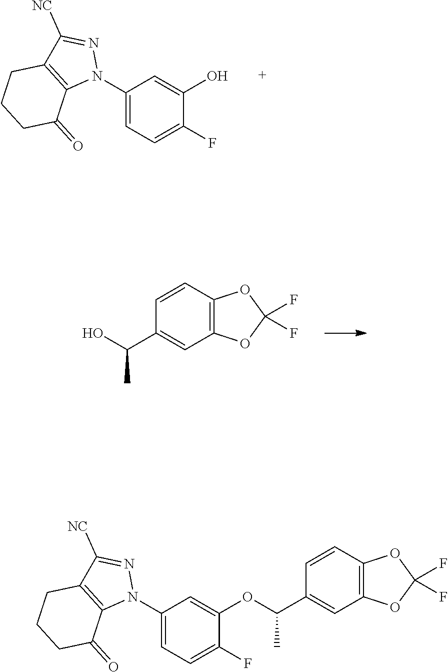 [SMILES: N#Cc1nn(-c2ccc(F)c(O)c2)c2c1CCCC2=O ; C[C@@H](O)c1ccc2c(c1)OC(F)(F)O2 ; C[C@H](Oc1cc(-n2nc(C#N)c3c2C(=O)CCC3)ccc1F)c1ccc2c(c1)OC(F)(F)O2]

To a solution of 1-(4-fluoro-3-hydroxy-phenyl)-7-oxo-5,6-dihydro-4H-indazole-3-carbonitrile (Intermediate D-1d) (1.00 eq, 150 mg, 0.553 mmol) and (1R)-1-(2,2-difluoro-1,3-benzodioxol-5-yl)ethanol (Intermediate C1R) (1.20 eq, 134 mg, 0.664 mmol) in THF (3 mL) were added triphenylphosphine (2.00 eq, 290 mg, 1.11 mmol), followed by diethyl azodicarboxylate (1.30 eq, 0.11 mL, 0.719 mmol) at 0° C. under N₂. The reaction was stirred at rt for 2 h. Then the reaction mixture was concentrated in vacuo. The residue was purified by silica gel column chromatography (0-20% ethyl acetate in heptane) to yield 1-[3-[(1S)-1-(2,2-difluoro-1,3-benzodioxol-5-yl)ethoxy]-4-fluoro-phenyl]-7-oxo-5,6-dihydro-4H-indazole-3-carbonitrile (145 mg, 0.318 mmol, 57.5% yield) as a yellow solid.

LCMS (Method 4): Retention time=2.20 min. MS (ESI) m/z 454.0 [M−H]⁺.

### Step 2. Synthesis of (R)-1-(3-((S)-1-(2,2-difluorobenzo[d][1,3]dioxol-5-yl)ethoxy)-4-fluorophenyl)-7-hydroxy-4,5,6,7-tetrahydro-1H-indazole-3-carbonitrile

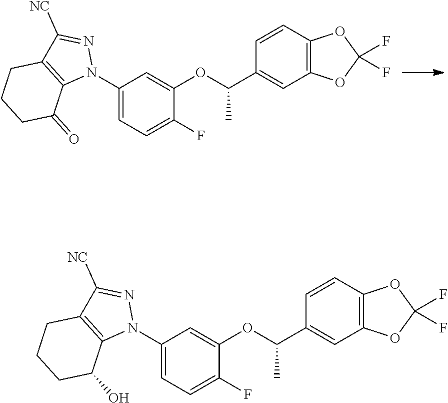 [SMILES: C[C@H](Oc1cc(-n2nc(C#N)c3c2C(=O)CCC3)ccc1F)c1ccc2c(c1)OC(F)(F)O2 ; C[C@H](Oc1cc(-n2nc(C#N)c3c2[C@H](O)CCC3)ccc1F)c1ccc2c(c1)OC(F)(F)O2]

To a solution of 1-[3-[(1S)-1-(2,2-difluoro-1,3-benzodioxol-5-yl)ethoxy]-4-fluoro-phenyl]-7-oxo-5,6-dihydro-4H-indazole-3-carbonitrile (1.00 eq, 145 mg, 0.318 mmol) in IPA (4 mL) were added and RuCl[(R,R)-tSDPEN](p-cymene) (5 mg), triethylamine (6.76 eq, 0.30 mL, 2.15 mmol), and formic acid (16.4 eq, 0.20 mL, 5.21 mmol) under N₂ at 0° C. The reaction was stirred at 45° C. for 4 h. Then the reaction mixture was concentrated in vacuo and the residue was purified by silica gel column chromatography (0-50% ethyl acetate in heptane) to yield (7R)-1-[3-[(1S)-1-(2,2-difluoro-1,3-benzodioxol-5-yl)ethoxy]-4-fluoro-phenyl]-7-hydroxy-4,5,6,7-tetrahydroindazole-3-carbonitrile (130 mg, 0.284 mmol, 89.2% yield) as a yellow solid.

LCMS (Method 4): Retention time=2.16 min. MS (ESI) m/z 458.0 [M+H]⁺.

### Step 3. Synthesis of methyl 4-(((S)-3-cyano-1-(3-((S)-1-(2,2-difluorobenzo[d][1,3]dioxol-5-yl)ethoxy)-4-fluorophenyl)-4,5,6,7-tetrahydro-1H-indazol-7-yl)oxy)benzoate

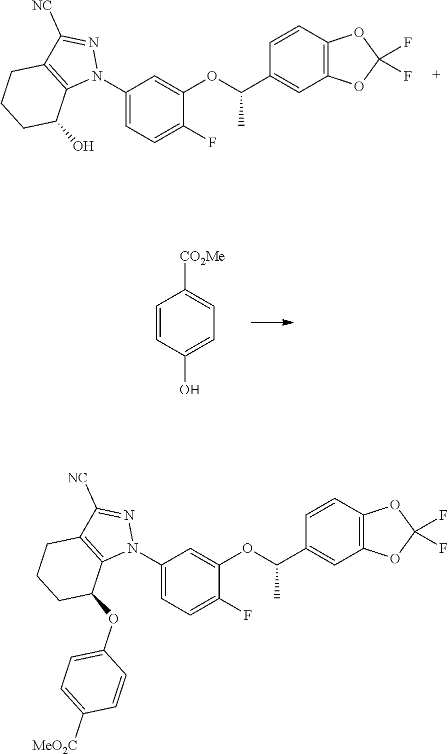 [SMILES: C[C@H](Oc1cc(-n2nc(C#N)c3c2[C@H](O)CCC3)ccc1F)c1ccc2c(c1)OC(F)(F)O2 ; COC(=O)c1ccc(O)cc1 ; COC(=O)c1ccc(O[C@H]2CCCc3c(C#N)nn(-c4ccc(F)c(O[C@@H](C)c5ccc6c(c5)OC(F)(F)O6)c4)c32)cc1]

To a solution of (7R)-1-[3-[(1S)-1-(2,2-difluoro-1,3-benzodioxol-5-yl)ethoxy]-4-fluoro-phenyl]-7-hydroxy-4,5,6,7-tetrahydroindazole-3-carbonitrile (1.00 eq, 80 mg, 0.175 mmol) in THF (3 mL) were added triphenylphosphine (2.00 eq, 92 mg, 0.350 mmol), methyl 4-hydroxybenzoate (1.20 eq, 32 mg, 0.210 mmol), and diethyl azodicarboxylate (1.30 eq, 0.036 mL, 0.227 mmol) at 0° C. under N₂. The reaction was stirred at rt for 1 h. Then the mixture was concentrated in vacuo. The residue was purified by silica gel column chromatography (0-15% ethyl acetate in heptane) to yield methyl 4-[[(7S)-3-cyano-1-[3-[(1S)-1-(2,2-difluoro-1,3-benzodioxol-5-yl)ethoxy]-4-fluoro-phenyl]-4,5,6,7-tetrahydroindazol-7-yl]oxy]benzoate (110 mg, 0.130 mmol, 74.4% yield) as a yellow solid.

LCMS (Method 4): Retention time=2.37 min. MS (ESI) m/z 592.1 [M+H]⁺.

### Step 4. Synthesis of 4-[[(7S)-3-cyano-1-[3-[(1S)-1-(2,2-difluoro-1,3-benzodioxol-5-yl)ethoxy]-4-fluoro-phenyl]-4,5,6,7-tetrahydroindazol-7-yl]oxy]benzoic acid

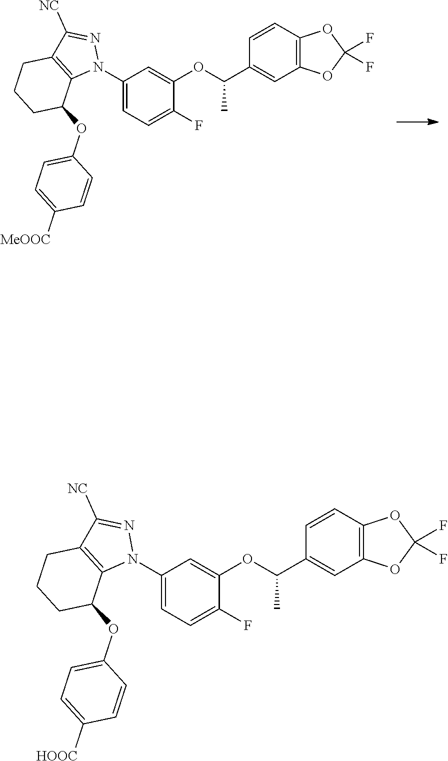 [SMILES: COC(=O)c1ccc(O[C@H]2CCCc3c(C#N)nn(-c4ccc(F)c(O[C@@H](C)c5ccc6c(c5)OC(F)(F)O6)c4)c32)cc1 ; C[C@H](Oc1cc(-n2nc(C#N)c3c2[C@@H](Oc2ccc(C(=O)O)cc2)CCC3)ccc1F)c1ccc2c(c1)OC(F)(F)O2]

To a solution of methyl 4-[[(7S)-3-cyano-1-[3-[(1S)-1-(2,2-difluoro-1,3-benzodioxol-5-yl)ethoxy]-4-fluoro-phenyl]-4,5,6,7-tetrahydroindazol-7-yl]oxy]benzoate (1.00 eq, 110 mg, 0.130 mmol) in THF (2 mL) and water (1 mL) was added lithium hydroxide (5.00 eq, 16 mg, 0.651 mmol) at rt and stirred for 18 h. To the reaction solution was added citric acid to adjust the pH to ˜3, then extracted with EA (20 mL×2). The combined organic solution was washed with brine (20 mL), then concentrated in vacuo. The residue was purified by prep-HPLC (0.01N NH₄HCO₃ in ACN) to yield 4-[[(7S)-3-cyano-1-[3-[(1S)-1-(2,2-difluoro-1,3-benzodioxol-5-yl)ethoxy]-4-fluoro-phenyl]-4,5,6,7-tetrahydroindazol-7-yl]oxy]benzoic acid (35 mg, 0.0606 mmol, 46.5% yield) as a white solid.

LCMS (Method 4): Retention time=1.72 min. MS (ESI) m/z 578.1 [M+H]⁺.

¹H NMR (400 MHZ, MeOH-d₄) δ:7.96 (d, J=8.8 Hz, 2H), 7.29-6.96 (m, 6H), 6.92 (d, J=8.8 Hz, 2H), 5.51 (s, 1H), 5.08 (q, J=6.3 Hz, 1H), 2.86 (dd, J=16.4, 2.7 Hz, 1H), 2.61 (ddd, J=16.5, 10.7, 5.9 Hz, 1H), 2.24 (d, J=12.4 Hz, 1H), 2.09-1.71 (m, 3H), 1.42 (d, J=6.3 Hz, 3H).

### Example 105 (P1 and P2)

### 3-[[(7S)-3-cyano-1-[3-[(1S)-1-(2,2-difluoro-1,3-benzodioxol-5-yl)ethoxy]-4-fluoro-phenyl]-4,5,6,7-tetrahydroindazol-7-yl]oxy]bicyclo[1.1.1]pentane-1-carboxylic acid

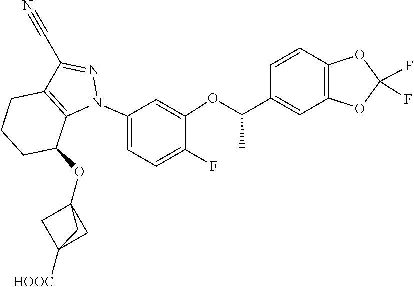 [SMILES: C[C@H](Oc1cc(-n2nc(C#N)c3c2[C@@H](OC24CC(C(=O)O)(C2)C4)CCC3)ccc1F)c1ccc2c(c1)OC(F)(F)O2]

and

### 3-[[(7R)-3-cyano-1-[3-[(1S)-1-(2,2-difluoro-1,3-benzodioxol-5-yl)ethoxy]-4-fluoro-phenyl]-4,5,6,7-tetrahydroindazol-7-yl]oxy]bicyclo[1.1.1]pentane-1-carboxylic acid

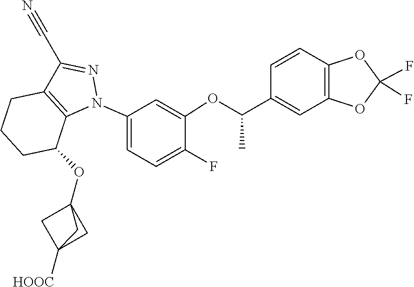 [SMILES: C[C@H](Oc1cc(-n2nc(C#N)c3c2[C@H](OC24CC(C(=O)O)(C2)C4)CCC3)ccc1F)c1ccc2c(c1)OC(F)(F)O2]

### Step 1. Synthesis of 7-chloro-1-(3-((S)-1-(2,2-difluorobenzo[d][1,3]dioxol-5-yl)ethoxy)-4-fluorophenyl)-4,5,6,7-tetrahydro-1H-indazole-3-carbonitrile

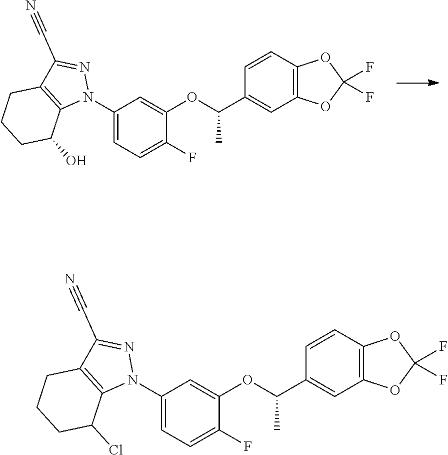 [SMILES: C[C@H](Oc1cc(-n2nc(C#N)c3c2[C@H](O)CCC3)ccc1F)c1ccc2c(c1)OC(F)(F)O2 ; C[C@H](Oc1cc(-n2nc(C#N)c3c2C(Cl)CCC3)ccc1F)c1ccc2c(c1)OC(F)(F)O2]

To a solution of (7R)-1-[3-[(1S)-1-(2,2-difluoro-1,3-benzodioxol-5-yl)ethoxy]-4-fluoro-phenyl]-7-hydroxy-4,5,6,7-tetrahydroindazole-3-carbonitrile (from step 2, Example 104) (1.00 eq, 220 mg, 0.481 mmol), triethylamine (2.50 eq, 0.17 mL, 1.20 mmol) in DCM (5 mL) was added methanesulfonyl chloride (1.50 eq, 0.056 mL, 0.721 mmol) at 0° C. The reaction was stirred at room temperature for 1 h. Then water (20 mL) was added, extracted with DCM (20 mL×2). The combined DCM solution was washed with brine (20 mL), dried (MgSO₄) and then concentrated in vacuo. The residue was purified by silica gel column chromatography (0-15% ethyl acetate in heptane) to yield 7-chloro-1-[3-[(1S)-1-(2,2-difluoro-1,3-benzodioxol-5-yl)ethoxy]-4-fluoro-phenyl]-4,5,6,7-tetrahydroindazole-3-carbonitrile (215 mg, 0.452 mmol, 93.9% yield) as yellow solid.

LCMS (Method 4): Retention time=2.40 min. MS (ESI) m/z 476.0 [M+H]⁺.

Step 2 and Step 3 were performed by following the same procedures as Example 74 (P1 and P2), Step 2 and Step 3.

### P1:

LCMS (Method 4): Retention time=1.67 min. MS (ESI) m/z 568.0 [M+H]⁺.

¹H NMR (400 MHZ, MeOH-d₄) δ:7.41-7.22 (m, 3H), 7.22-7.14 (m, 2H), 7.14-7.04 (m, 1H), 5.55 (q, J=6.3 Hz, 1H), 4.49 (t, J=3.3 Hz, 1H), 2.73 (dt, J=16.1, 4.0 Hz, 1H), 2.51 (dt, J=16.4, 8.2 Hz, 1H), 2.12-1.95 (m, 1H), 1.94-1.81 (m, 5H), 1.81-1.70 (m, 4H), 1.67 (t, J=7.0 Hz, 3H).

P2: LCMS (Method 4): Retention time=1.67 min. MS (ESI) m/z 568.0 [M+H]⁺.

¹H NMR (400 MHZ, MeOH-d₄) δ:7.37-7.25 (m, 2H), 7.21 (ddd, J=13.4, 7.8, 2.0 Hz, 2H), 7.17-7.06 (m, 2H), 5.58 (q, J=6.3 Hz, 1H), 4.73 (t, J=3.2 Hz, 1H), 2.80-2.64 (m, 1H), 2.61-2.40 (m, 1H), 2.02 (dt, J=27.6, 12.4 Hz, 1H), 1.95-1.71 (m, 9H), 1.66 (d, J=6.4 Hz, 3H).

### Example 106 (P1 and P2)

### 3-[[(7S)-3-cyano-1-[3-[[(1S)-1-(2,2-difluoro-1,3-benzodioxol-5-yl)ethyl]amino]-4-fluoro-phenyl]-4,5,6,7-tetrahydroindazol-7-yl]oxy]bicyclo[1.1.1]pentane-1-carboxylic acid

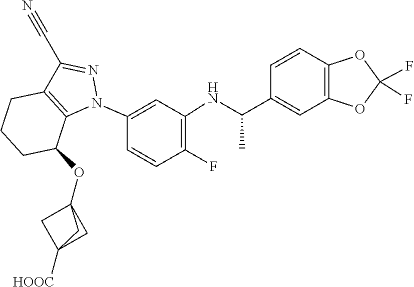 [SMILES: C[C@H](Nc1cc(-n2nc(C#N)c3c2[C@@H](OC24CC(C(=O)O)(C2)C4)CCC3)ccc1F)c1ccc2c(c1)OC(F)(F)O2]

and

### 3-[[(7R)-3-cyano-1-[3-[[(1S)-1-(2,2-difluoro-1,3-benzodioxol-5-yl)ethyl]amino]-4-fluoro-phenyl]-4,5,6,7-tetrahydroindazol-7-yl]oxy]bicyclo[1.1.1]pentane-1-carboxylic acid

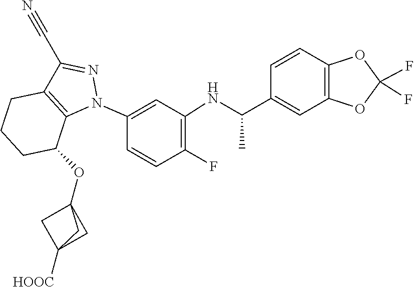 [SMILES: C[C@H](Nc1cc(-n2nc(C#N)c3c2[C@H](OC24CC(C(=O)O)(C2)C4)CCC3)ccc1F)c1ccc2c(c1)OC(F)(F)O2]

### Step 1. Synthesis of (R)-1-(3-bromo-4-fluorophenyl)-7-hydroxy-4,5,6,7-tetrahydro-1H-indazole-3-carbonitrile

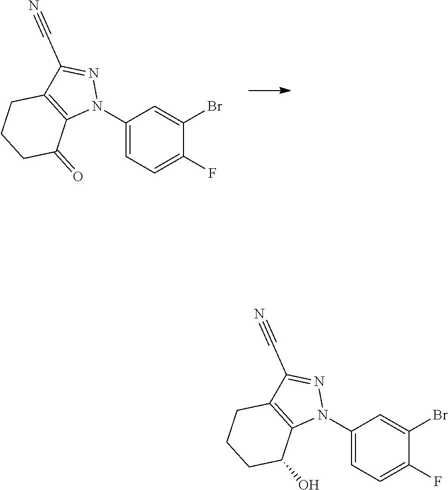 [SMILES: N#Cc1nn(-c2ccc(F)c(Br)c2)c2c1CCCC2=O ; N#Cc1nn(-c2ccc(F)c(Br)c2)c2c1CCC[C@H]2O]

To a solution of 1-(3-bromo-4-fluoro-phenyl)-7-oxo-5,6-dihydro-4H-indazole-3-carbonitrile (Intermediate D-12a) (1100 mg, 3.29 mmol) in IPA (15 mL) were added RuCl (p-cymene) [(R,R)-Ts-DPEN] (CAS No.: 192139-92-7) (100 mg, 0.16 mmol), triethylamine (3.0 mL), and formic acid (2.0 mL) at 0° C. under N₂. The reaction was stirred at 45° C. for 1 h. The mixture was concentrated in vacuo. The residue was purified by column chromatography on silica gel (0-20% ethyl acetate in heptane) to yield (7R)-1-(3-bromo-4-fluoro-phenyl)-7-hydroxy-4,5,6,7-tetrahydroindazole-3-carbonitrile (1030 mg, 93.1% yield) as a yellow solid.

LCMS (Method 4): Retention time=1.90 min. MS (ESI) m/z 336.0 [M+H]⁺.

### Step 2. Synthesis of 7-bromo-1-(3-bromo-4-fluorophenyl)-4,5,6,7-tetrahydro-1H-indazole-3-carbonitrile

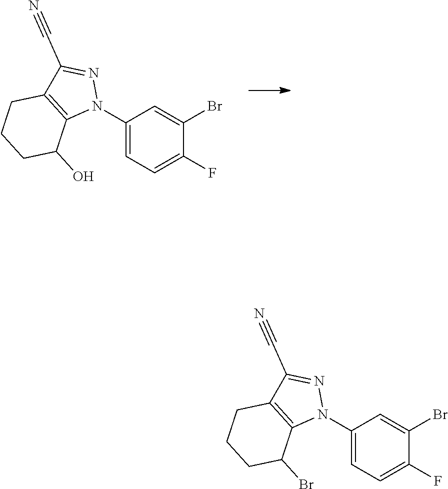 [SMILES: N#Cc1nn(-c2ccc(F)c(Br)c2)c2c1CCCC2O ; N#Cc1nn(-c2ccc(F)c(Br)c2)c2c1CCCC2Br]

To a solution of 1-(3-bromo-4-fluoro-phenyl)-7-hydroxy-4,5,6,7-tetrahydroindazole-3-carbonitrile (1000 mg, 2.97 mmol) in DCM (10 mL) was added phosphorus tribromide (0.42 mL, 4.46 mmol) at 0° C. The reaction was stirred at rt for 2 h. Then water (20 mL) was added and extracted with DCM (20 mL×2). The combined DCM solution was washed with brine (20 mL), dried (MgSO₄), filtered, and concentrated in vacuo. The residue was purified by column chromatography (0-10% ethyl acetate in heptane) to yield 7-bromo-1-(3-bromo-4-fluoro-phenyl)-4,5,6,7-tetrahydroindazole-3-carbonitrile (840 mg, 70.7%) as a brown oil. LCMS (Method 4): Retention time=2.18 min. MS (ESI) m/z 397.8 [M+H]⁺.

### Step 3. Synthesis of methyl 3-((1-(3-bromo-4-fluorophenyl)-3-cyano-4,5,6,7-tetrahydro-1H-indazol-7-yl)oxy)bicyclo[1.1.1]pentane-1-carboxylate

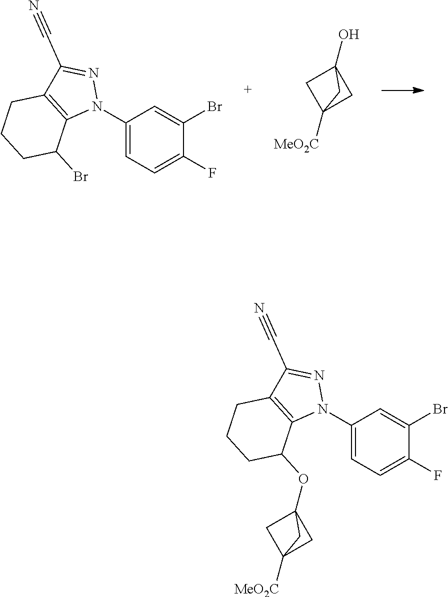 [SMILES: N#Cc1nn(-c2ccc(F)c(Br)c2)c2c1CCCC2Br ; COC(=O)C12CC(O)(C1)C2 ; COC(=O)C12CC(OC3CCCc4c(C#N)nn(-c5ccc(F)c(Br)c5)c43)(C1)C2]

To a solution of methyl 3-hydroxybicyclo[1.1.1]pentane-1-carboxylate (329 mg, 2.32 mmol), 2,6-di-tert-butylpyridine (805 mg, 4.21 mmol) in DCM (10 mL) were added silver trifluoromethanesulfonate (649 mg, 2.53 mmol), 7-bromo-1-(3-bromo-4-fluoro-phenyl)-4,5,6,7-tetrahydroindazole-3-carbonitrile (840 mg, 2.10 mmol) at −20° C. under N₂. The reaction was stirred at room temperature for 8 h. Then water (20 mL) was added and extracted with DCM (20 mL×2). The combined DCM solution was washed with brine (20 mL), dried (MgSO₄), filtered, and concentrated in vacuo. The residue was purified by column chromatography on silica gel (0-10% ethyl acetate in heptane) to yield methyl 3-[[1-(3-bromo-4-fluoro-phenyl)-3-cyano-4,5,6,7-tetrahydroindazol-7-yl]oxy]bicyclo[1.1.1]pentane-1-carboxylate (420 mg, 43.5%) as a yellow oil. LCMS (Method 4): Retention time=2.13 min. MS (ESI) m/z 460.0, 462.0 [M+H]⁺.

### Step 4. Synthesis of methyl 3-[[(7S)-3-cyano-1-[3-[[(1S)-1-(2,2-difluoro-1,3-benzodioxol-5-yl)ethyl]amino]-4-fluoro-phenyl]-4,5,6,7-tetrahydroindazol-7-yl]oxy]bicyclo[1.1.1]pentane-1-carboxylate and methyl 3-[[(7R)-3-cyano-1-[3-[[(1S)-1-(2,2-difluoro-1,3-benzodioxol-5-yl)ethyl]amino]-4-fluoro-phenyl]-4,5,6,7-tetrahydroindazol-7-yl]oxy]bicyclo[1.1.1]pentane-1-carboxylate

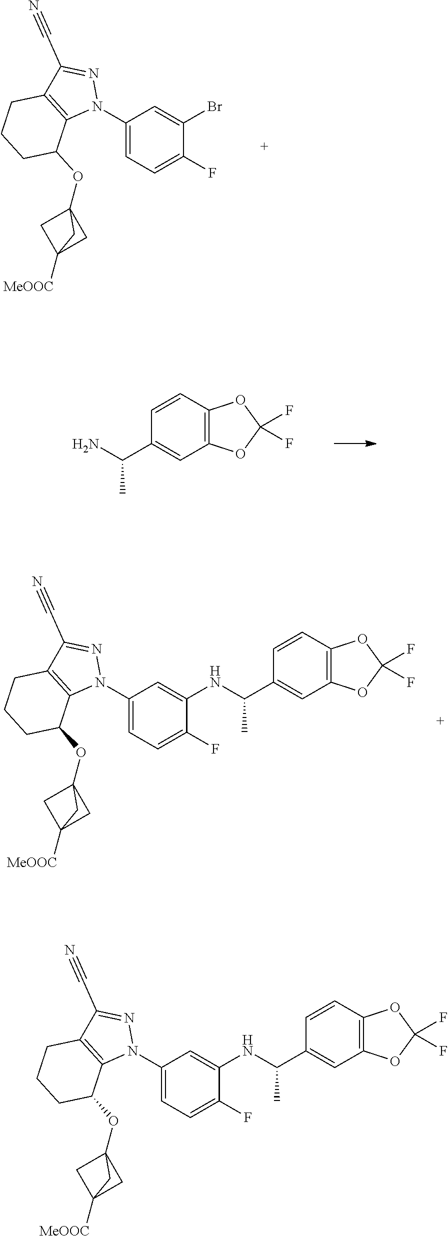 [SMILES: COC(=O)C12CC(OC3CCCc4c(C#N)nn(-c5ccc(F)c(Br)c5)c43)(C1)C2 ; C[C@H](N)c1ccc2c(c1)OC(F)(F)O2 ; COC(=O)C12CC(O[C@H]3CCCc4c(C#N)nn(-c5ccc(F)c(N[C@@H](C)c6ccc7c(c6)OC(F)(F)O7)c5)c43)(C1)C2 ; COC(=O)C12CC(O[C@@H]3CCCc4c(C#N)nn(-c5ccc(F)c(N[C@@H](C)c6ccc7c(c6)OC(F)(F)O7)c5)c43)(C1)C2]

To a solution of methyl 3-[[1-(3-bromo-4-fluoro-phenyl)-3-cyano-4,5,6,7-tetrahydroindazol-7-yl]oxy]bicyclo[1.1.1]pentane-1-carboxylate (1.00 eq, 420 mg, 0.912 mmol) in 1,4-dioxane (8 mL) were added (1S)-1-(2,2-difluoro-1,3-benzodioxol-5-yl)ethanamine (Intermediate C-6a) (1.20 eq, 220 mg, 1.09 mmol), cesium carbonate (2.00 eq, 595 mg, 1.82 mmol), and GPhos Pd G3 (CAS No.: 2489525-82-6) (0.0604 eq, 50 mg, 0.0552 mmol) under N₂. The reaction was stirred at 100° C. for 1 h. Then water (20 mL) was added, extracted with EA (20 mL×2). The combined EA solution was washed with brine (20 mL), dried (Na₂SO₄), filtered, and concentrated in vacuo. The residue was purified by column chromatography on silica gel (0-25% ethyl acetate in heptane) to give the crude compound (150 mg). This crude product was separated by SFC to yield methyl 3-[[(7S)-3-cyano-1-[3-[[(1S)-1-(2,2-difluoro-1,3-benzodioxol-5-yl)ethyl]amino]-4-fluoro-phenyl]-4,5,6,7-tetrahydroindazol-7-yl]oxy]bicyclo[1.1.1]pentane-1-carboxylate (65 mg, 0.112 mmol, 12.2% yield) as a white solid and methyl 3-[[(7R)-3-cyano-1-[3-[[(1S)-1-(2,2-difluoro-1,3-benzodioxol-5-yl)ethyl]amino]-4-fluoro-phenyl]-4,5,6,7-tetrahydroindazol-7-yl]oxy]bicyclo[1.1.1]pentane-1-carboxylate (60 mg, 0.103 mmol, 11.3% yield) as a white solid.

LC-MS (Method 4): Retention time=2.27 min. MS (ESI) m/z 581.1 [M+H]⁺.

### Step 5. Synthesis of 3-[[(7S)-3-cyano-1-[3-[[(1S)-1-(2,2-difluoro-1,3-benzodioxol-5-yl)ethyl]amino]-4-fluoro-phenyl]-4,5,6,7-tetrahydroindazol-7-yl]oxy]bicyclo[1.1.1]pentane-1-carboxylic acid and 3-[[(7R)-3-cyano-1-[3-[[(1S)-1-(2,2-difluoro-1,3-benzodioxol-5-yl)ethyl]amino]-4-fluoro-phenyl]-4,5,6,7-tetrahydroindazol-7-yl]oxy]bicyclo[1.1.1]pentane-1-carboxylic acid

The title compounds were obtained by hydrolysis of the esters following the protocol of Step 7a and 7b of Example 94 (P1 and P2).

### P1:

LCMS (Method 4): Retention time=1.65 min. MS (ESI) m/z 567.0 [M+H]⁺.

¹H NMR (400 MHZ, MeOH-d₄) δ:7.31 (d, J=1.5 Hz, 1H), 7.23 (dt, J=12.8, 6.4 Hz, 1H), 7.19-7.05 (m, 2H), 6.68 (ddd, J=8.4, 3.9, 2.5 Hz, 1H), 6.53 (dd, J=7.6, 2.5 Hz, 1H), 4.63-4.46 (m, 1H), 4.28 (t, J=3.0 Hz, 1H), 2.71 (dt, J=16.2, 4.0 Hz, 1H), 2.57-2.40 (m, 1H), 1.96 (dd, J=14.2, 3.0 Hz, 1H), 1.88-1.73 (m, 5H), 1.67 (dd, J=15.4, 7.3 Hz, 4H), 1.57 (dd, J=17.0, 5.1 Hz, 3H).

P2: LCMS (Method 4): Retention time=1.65 min. MS (ESI) m/z 567.0 [M+H]⁺.

¹H NMR (400 MHZ, MeOH-d₄) δ:7.33-7.20 (m, 1H), 7.14 (ddd, J=18.3, 11.8, 7.5 Hz, 3H), 6.67 (ddd, J=8.4, 3.9, 2.6 Hz, 1H), 6.54 (dd, J=7.6, 2.5 Hz, 1H), 4.74 (d, J=3.2 Hz, 1H), 4.63 (q, J=6.7 Hz, 1H), 2.69 (t, J=9.9 Hz, 1H), 2.55-2.40 (m, 1H), 2.06-1.93 (m, 1H), 1.87-1.67 (m, 9H), 1.55 (d, J=6.8 Hz, 3H).

Examples 107 (P1 and P2) and Examples 108 (P1 and P2) were synthesized in the similar manner as Example 106 (P1 and P2)

| Ex. No. | Structure | Name | LCMS/NMR |
|---|---|---|---|
| 107 (P1) | 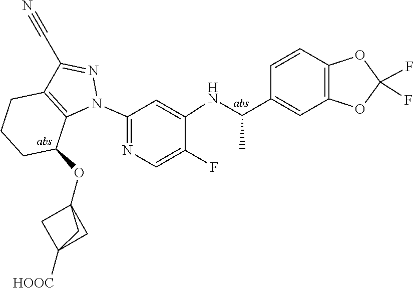 [SMILES: C[C@H](Nc1cc(-n2nc(C#N)c3c2[C@@H](OC24CC(C(=O)O)(C2)C4)CCC3)ncc1F)c1ccc2c(c1)OC(F)(F)O2] | 3-[[(7S)-3-cyano-1-[4-[(1S)-1- (2,2-difluoro-1,3-benzodioxol- 5-yl)ethyl]amino]-5-fluoro-2- pyridyl]-4,5,6,7- tetrahydroindazol-7- yl]oxy]bicyclo[1.1.1]pentane- 1-carboxylic acid | LCMS (Method 4): Retention time = 1.72 min. MS (ESI) m/z 568.1 [M + H]⁺. ¹H NMR (400 MHZ, MeOH-d₄) δ: 8.04 (d, J = 3.3 Hz, 1H), 7.25 (d, J = 1.6 Hz, 1H), 7.21 (dd, J= 8.3, 1.6 Hz, 1H), 7.13 (d, J = 8.3 Hz, 1H), 6.73 (d, J = 6.4 Hz, 1H), 5.42 (s, 1H), 4.78-4.68 (m, 1H), 2.68 (d, J = 15.9 Hz, 1H), 2.55-2.37 (m, 1H), 2.03 (d, J = 9.6 Hz, 1H), 1.99-1.90 (m, 3H), 1.90-1.69 (m, 6H), 1.59 (d, J = 6.8 Hz, 3H). |
| 107 (P2) | 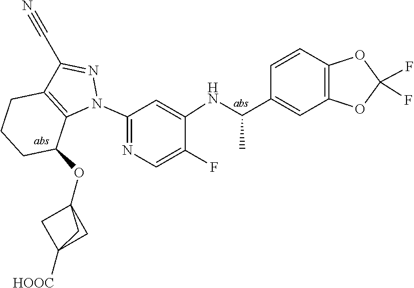 [SMILES: C[C@H](Nc1cc(-n2nc(C#N)c3c2[C@@H](OC24CC(C(=O)O)(C2)C4)CCC3)ncc1F)c1ccc2c(c1)OC(F)(F)O2] | 3-[[(7R)-3-cyano-1-[4-[[(1S)-1- (2,2-difluoro-1,3-benzodioxol- 5-yl)ethyl]amino]-5-fluoro-2- pyridyl]-4,5,6,7- tetrahydroindazol-7- yl]oxy]bicyclo[1.1.1]pentane- 1-carboxylic acid | LCMS (Method 4): Retention time = 1.74 min. MS (ESI) m/z 568.0 [M + H]⁺. ¹H NMR (400 MHZ, MeOH-d₄) δ: 8.04 (d, J = 3.3 Hz, 1H), 7.30-7.22 (m, 2H), 7.17 (d, J = 8.3 Hz, 1H), 6.83 (d, J = 6.4 Hz, 1H), 5.35 (d, J = 3.3 Hz, 1H), 4.74 (q, J = 6.9 Hz, 1H), 2.69 (d, J = 16.1 Hz, 1H), 2.57-2.40 (m, 1H), 2.07 (d, J = 11.1 Hz, 1H), 1.98 (d, J = 9.1 Hz, 3H), 1.89-1.71 (m, 6H), 1.58 (d, J = 6.8 Hz, 3H). |
| 108(P1) | 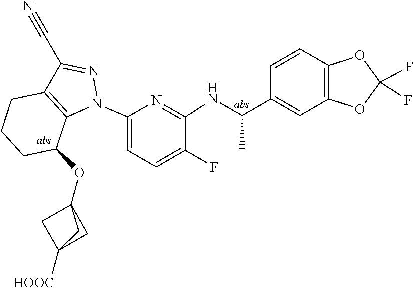 [SMILES: C[C@H](Nc1nc(-n2nc(C#N)c3c2[C@@H](OC24CC(C(=O)O)(C2)C4)CCC3)ccc1F)c1ccc2c(c1)OC(F)(F)O2] | 3-[[(7S)-3-cyano-1-[6-[(1S)-1- (2,2-difluoro-1,3-benzodioxol- 5-yl)ethyl]amino]-5-fluoro-2- pyridyl]-4,5,6,7- tetrahydroindazol-7- yl]oxy]bicyclo[1.1.1]pentane- 1-carboxylic acid | LCMS (Method 4): Retention time = 1.61 min. MS (ESI) m/z 568.0 [M + H]⁺. ¹H NMR (400 MHZ, MeOH-d₄) δ: 7.46 (dd, J = 10.5, 8.1 Hz, 1H), 7.35 (d, J = 1.5 Hz, 1H), 7.28 (dd, J = 8.3, 1.6 Hz, 1H), 7.18 (d, J = 8.2 Hz, 1H), 6.67 (dd, J = 8.1, 2.6 Hz, 1H), 5.07 (q, J = 6.9 Hz, 1H), 4.54 (s, 1H), 2.71 (t, J = 9.6 Hz, 1H), 2.59-2.44 (m, 1H), 1.91-1.66 (m, 4H), 1.50 (dd, J = 13.6, 4.1 Hz, 6H), 1.34 (dd, J = 9.3, 1.2 Hz, 3H). |
| 108 (P2) | 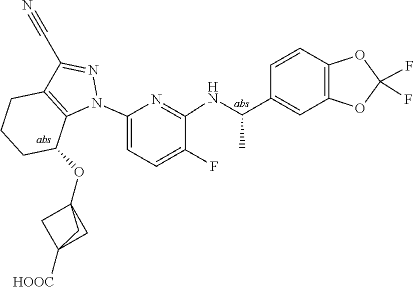 [SMILES: C[C@H](Nc1nc(-n2nc(C#N)c3c2[C@H](OC24CC(C(=O)O)(C2)C4)CCC3)ccc1F)c1ccc2c(c1)OC(F)(F)O2] | 3-[[(7R)-3-cyano-1-[6-[(1S)-1- (2,2-difluoro-1,3-benzodioxol- 5-yl)ethyl]amino]-5-fluoro-2- pyridyl]-4,5,6,7- tetrahydroindazol-7- yl]oxy]bicyclo[1.1.1]pentane- 1-carboxylic acid | LCMS (Method 4): Retention time = 1.62 min. MS (ESI) m/z 568.1 [M + H]⁺. ¹H NMR (400 MHZ, MeOH-d₄) δ: 7.42 (dt, J = 29.5, 14.8 Hz, 1H), 7.19 (dd, J = 12.7, 4.7 Hz, 1H), 7.13 (dd, J = 8.3, 1.6 Hz, 1H), 7.02 (d, J = 8.3 Hz, 1H), 6.70 (dt, J = 16.4, 8.2 Hz, 1H), 5.45 (t, J = 3.5 Hz, 1H), 5.22 (q, J = 7.1 Hz, 1H), 2.79-2.62 (m, 1H), 2.62-2.42 (m, 1H), 2.10 (dd, J = 13.7, 3.8 Hz, 1H), 2.05- 1.76 (m, 9H), 1.60 (d, J = 7.1 Hz, 3H). |

### Example 109

### 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]amino]benzoic acid

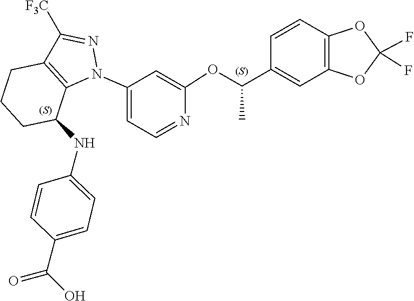

### Step 1. Synthesis of 2-[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]isoindoline-1,3-dione

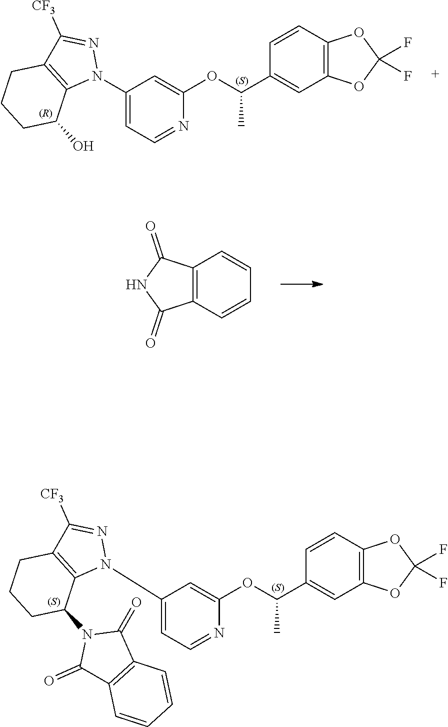

To a solution of (7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-ol (from Step 5, Example 24, Synthetic method 2) (1.00 g, 2.07 mmol) in dry THF (8 mL) were added phthalimide (457 mg, 3.10 mmol) and triphenylphosphine (814 mg, 3.10 mmol). The reaction was stirred at 0° C. under a nitrogen atmosphere. To this mixture was added dropwise diethyl azodicarboxylate (0.49 mL, 3.10 mmol) over a period of 10 min, and the reaction was monitored by LCMS. After complete, the reaction mixture was concentrated and purified by reversed-phase flash chromatography (C18) (0.1% NH₄HCO₃, 10-100% MeCN) to obtain 2-[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]isoindoline-1,3-dione (1.20 g, 1.96 mmol, 94.70% yield) as a white solid.

LCMS (Method 2): Retention time=2.55 min. MS (ESI) m/z 429.0 [M-184+H]⁺.

### Step 2. Synthesis of (7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-amine

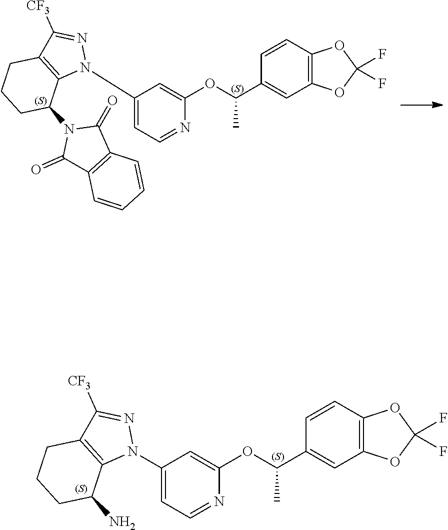

To a solution of 2-[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]isoindoline-1,3-dione (1.20 g, 1.96 mmol) in methanol (1.5 mL) and THF (1.5 mL) was added hydrazine hydrate (981 mg, 19.6 mmol) (N₂H₄·H₂O) at 0° C. The reaction was stirred at 80° C. for 5 h. The reaction mixture was concentrated and the residue was purified by reversed-phase flash chromatography (C18) (0.1% NH₄HCO₃ in water, 10-100% MeCN) to obtain (7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-amine (800 mg, 1.66 mmol, 84.6% yield) as a colorless oil.

LCMS (Method 1): Retention time=1.77 min. MS (ESI) m/z 299.0 [M-184+H]⁺.

Chiral Method: Column Name: OX 4.6*100 mm 5 μm Solvent: MeOH [0.2% NH₃ (7M in MeOH)]; Find at 2.046 min; ee: 99.16%

### Step3. Synthesis of methyl 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]amino]benzoate

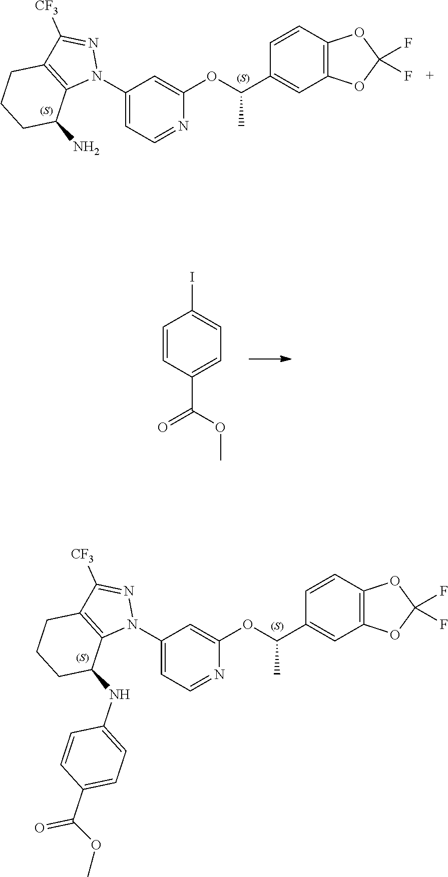

To a solution of (7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-amine (180 mg, 0.373 mmol) and methyl 4-iodobenzoate (147 mg, 0.560 mmol) in 1,4-dioxane (10 mL) were added cesium carbonate (365 mg, 1.12 mmol) and GPhos Pd G3 (CAS No.: 2489525-82-6) (34 mg, 0.0373 mmol). The reaction mixture was stirred at 100° C. for 1.5 h under a nitrogen atmosphere. The mixture was concentrated and purified by flash column chromatography (PE:EA=5:1) to give the crude product. The crude product purified by Reversed-phase flash chromatography (C18) (0.1% NH₄HCO₃ in water, 10-100% MeCN) to obtain methyl 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]amino]benzoate (180 mg, 0.292 mmol, 78.24% yield) as a white solid.

LCMS (Method 1): Retention time=2.44 min. MS (ESI) m/z 433.0 [M-184+H]⁺.

Chiral Method: Column Name: AD-H 4.6*100 mm 5 μm Solvent: MeOH [0.2% NH3 (7M in MeOH)]; Find at 1.667 min; ee: 98.6%

### Step 4. Synthesis of 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]amino]benzoic acid

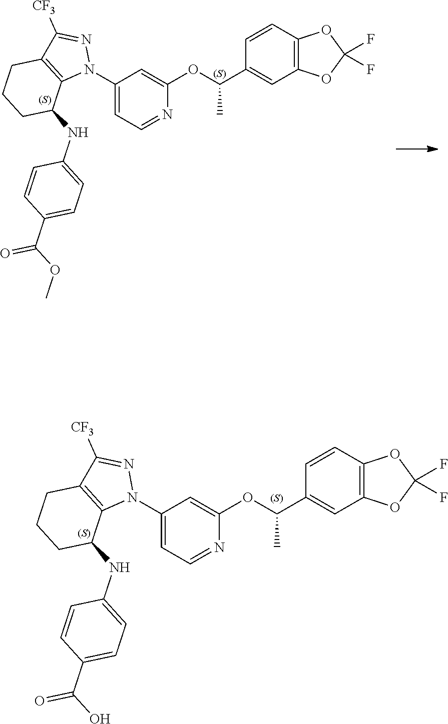

To a solution of methyl 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]amino] (80 mg, 0.130 mmol) in methanol (1.5 mL) and THF (1.5 mL) was added sodium hydroxide (1.9 mL, 3.89 mmol) at rt. The reaction mixture was stirred at 50° C. for 1 h. The reaction was monitored by LCMS. After completion, the reaction mixture was adjusted to pH 5-6 by addition of citric acid. Then the mixture was diluted with water (10 mL) and extracted with ethyl acetate (20 mL×3). The organic phase was washed with brine and dried over Na₂SO₄, filtered, and concentrated under vacuum. The residue was purified by reversed-phase flash chromatography (C18) (0.1% NH₄HCO₃ in water, 10-100% MeCN) to give 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]amino]benzoic acid (31 mg, 0.0511 mmol, 39.4% yield) as a white solid.

LCMS (Method 2): Retention time=1.87 min. MS (ESI) m/z 601.0 [M−H]⁻.

Chiral Method: Column Name: OX 4.6*100 mm 5 μm Solvent: MeOH [0.2% NH₃ (7M in MeOH)]; Find at 1.796 min; ee: 100%

¹H NMR (400 MHZ, MeOH-d₄) δ 8.04 (d, J=5.6 Hz, 1H), 7.86 (d, J=8.7 Hz, 2H), 7.23 (dd, J=5.7, 1.8 Hz, 1H), 7.12 (dd, J=19.2, 4.7 Hz, 3H), 7.00 (d, J=8.4 Hz, 1H), 6.69 (d, J=8.8 Hz, 2H), 5.96 (q, J=6.5 Hz, 1H), 4.97 (s, 1H), 2.89-2.78 (m, 1H), 2.69-2.54 (m, 1H), 2.16-2.06 (m, 1H), 2.03-1.76 (m, 3H), 1.42 (d, J=6.5 Hz, 3H).

### Example 110

### 4-[[(7S)-1-[2-[(1S)-1-(2,2,6-trifluoro-1,3-benzodioxol-5-yl)ethoxy]-4-pyridyl]-3-(trifluoromethyl)-4,5,6,7-tetrahydroindazol-7-yl]amino]benzoic acid

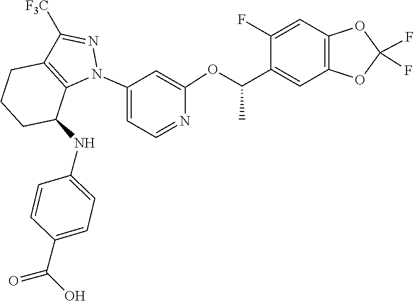 [SMILES: C[C@H](Oc1cc(-n2nc(C(F)(F)F)c3c2[C@@H](Nc2ccc(C(=O)O)cc2)CCC3)ccn1)c1cc2c(cc1F)OC(F)(F)O2]

Example 110 was synthesized similarly as Example 109 (31 mg, 0.0488 mmol, 61.96% yield) as a white solid.

LCMS (Method 2): Retention time=1.90 min. MS (ESI) m/z 619.0 [M−H]⁻. Chiral Method: Column Name: IG 4.6*100 mm 5 μm Solvent: MeOH [0.2% NH₃ (7M in MeOH)]; Find at 2.319 min; ee: 98.82%

¹H NMR (400 MHZ, MeOH-d₄) δ 8.02 (d, J=5.6 Hz, 1H), 7.83 (d, J=8.7 Hz, 2H), 7.23 (dd, J=5.6, 1.8 Hz, 1H), 7.16 (d, J=1.5 Hz, 1H), 7.05 (dd, J=16.1, 7.3 Hz, 2H), 6.67 (d, J=8.7 Hz, 2H), 6.31-6.20 (m, 1H), 5.00 (s, 1H), 2.83 (d, J=16.3 Hz, 1H), 2.70-2.55 (m, 1H), 2.09 (d, J=11.0 Hz, 1H), 2.03-1.77 (m, 3H), 1.43 (d, J=6.5 Hz, 3H).

### Example 111

### 3-[4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]phenyl]-2H-1,2,4-oxadiazol-5-one

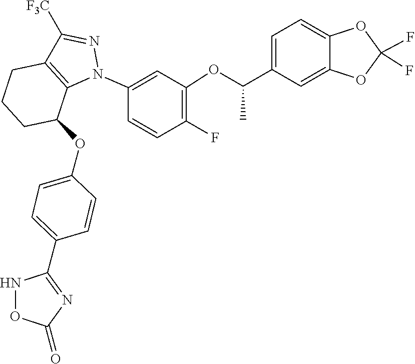 [SMILES: C[C@H](Oc1cc(-n2nc(C(F)(F)F)c3c2[C@@H](Oc2ccc(-c4nc(=O)o[nH]4)cc2)CCC3)ccc1F)c1ccc2c(c1)OC(F)(F)O2]

### Step 1. Synthesis of 4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]benzonitrile

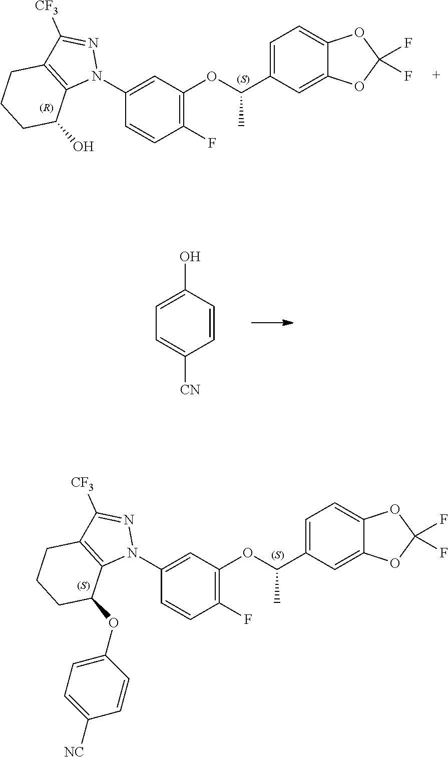

To a solution of (7R)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-ol (Intermediate obtained in Step 2, Example 36) (150 mg, 0.300 mmol), 4-hydroxybenzonitrile (43 mg, 0.360 mmol) and triphenylphosphine (118 mg, 0.450 mmol) was stirred in dry THF (3 mL) at 0° C. under a nitrogen atmosphere. To this mixture was added drop wise diethyl azodicarboxylate (0.071 mL, 0.450 mmol) over a period of 10 min, and the reaction was monitored by LCMS. After complete, the reaction mixture was concentrated and purified by Reversed-phase flash chromatography (C18) (0.1% NH₄HCO₃, 10-100% MeCN) to give 4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]benzonitrile (110 mg, 0.183 mmol, 61.0% yield) as a white solid.

LCMS (Method 1): Retention time=2.37 min. MS (ESI) m/z 602.2 [M+H]⁺.

### Step 2. Synthesis of 4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-N′-hydroxy-benzamidine

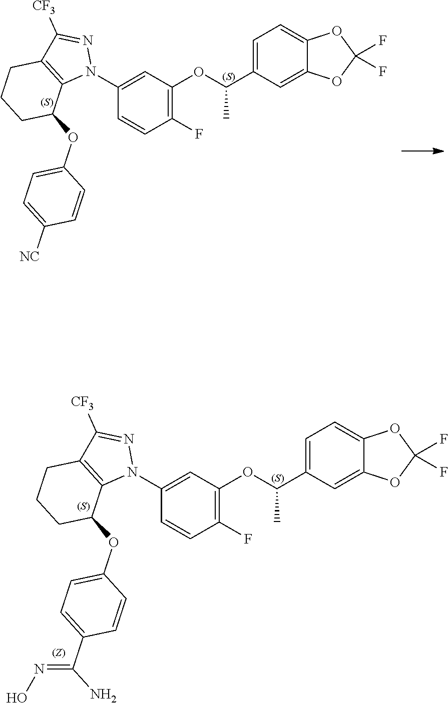

To a solution of 4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]benzonitrile (110 mg, 0.183 mmol) in methanol (1.5 mL) and THF (1.5 mL) was added hydroxylamine (6.0 mg, 0.183 mmol) (50% NH₂OH aq). The reaction mixture was stirred at 80° C. for 1 h. The reaction was diluted with water (10 mL) and extracted with ethyl acetate (20 mL×3). The organic phase was washed with brine and dried over Na₂SO₄ and concentrated under vacuum. The residue was purified by reversed-phase flash chromatography (C18) (0.1% NH₄HCO₃ in water, 10-100% acetonitrile) to give 4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-N′-hydroxy-benzamidine (100 mg, 0.158 mmol, 86.1% yield) as a white solid.

LCMS (Method 1): Retention time=1.87 min. MS (ESI) m/z 635.0 [M+H]⁺.

### Step 3. Synthesis of 3-[4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]phenyl]-2H-1,2,4-oxadiazol-5-one

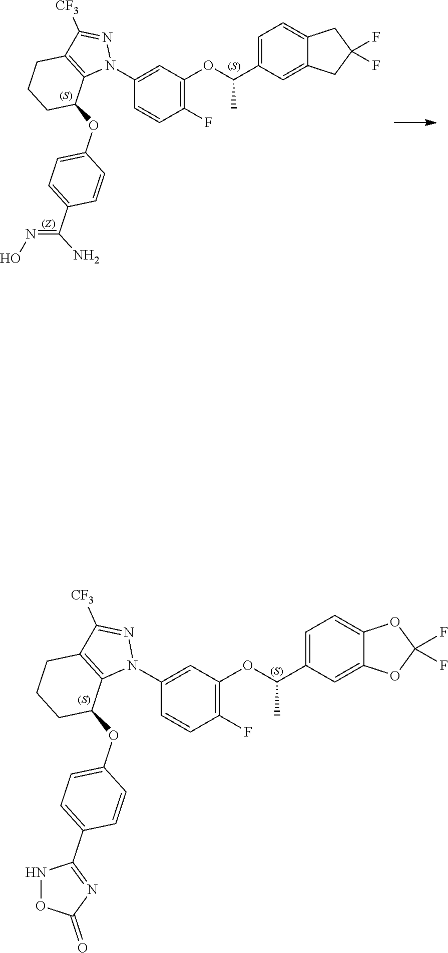

To a solution of 4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]-N′-hydroxy-benzamidine (80 mg, 0.126 mmol) in ethyl acetate (2 mL) was added N,N′-carbonyldiimidazole (102 mg, 0.630 mmol). Then the reaction mixture was stirred at 60° C. for 5 h. The reaction was diluted with water (10 mL) and extracted with ethyl acetate (20 mL×3). The organic phase was washed with brine, dried over Na₂SO₄ and concentrated under vacuum. The residue was purified by reversed-phase flash chromatography (C18) (0.1% NH₄HCO₃ in water, 10-100% MeCN) to give 3-[4-[[(7S)-1-[3-[(1S)-1-(2,2-difluoro-1,3-benzodioxol-5-yl)ethoxy]-4-fluoro-phenyl]-3-(trifluoromethyl)-4,5,6,7-tetrahydroindazol-7-yl]oxy]phenyl]-2H-1,2,4-oxadiazol-5-one (28 mg, 0.0418 mmol, 33.14% yield) as a white solid.

LCMS (Method 2): Retention time=1.84 min. MS (ESI) m/z 659.0 [M−H]⁻.

¹H NMR (400 MHZ, MeOH-d₄) δ 7.77-7.68 (m, 2H), 7.19-7.04 (m, 6H), 7.02-6.96 (m, 2H), 5.49 (t, J=3.0 Hz, 1H), 5.14 (q, J=6.3 Hz, 1H), 2.86 (d, J=16.4 Hz, 1H), 2.67-2.55 (m, 1H), 2.22 (d, J=11.9 Hz, 1H), 2.05-1.81 (m, 3H), 1.45 (d, J=6.4 Hz, 3H).

### Example 112

### 3-[4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]oxy]phenyl]-2H-1,2,4-oxadiazol-5-one

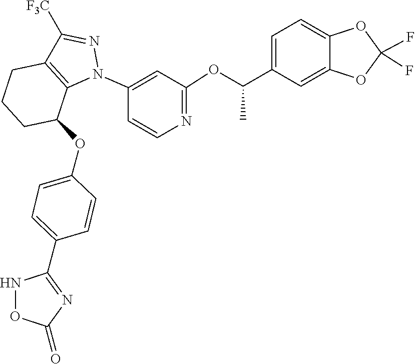 [SMILES: C[C@H](Oc1cc(-n2nc(C(F)(F)F)c3c2[C@@H](Oc2ccc(-c4nc(=O)o[nH]4)cc2)CCC3)ccn1)c1ccc2c(c1)OC(F)(F)O2]

Example 112 was synthesized similarly to Example 111 (27 mg, 0.0412 mmol, 50.86% yield) as a white solid.

LCMS (Method 2): Retention time=1.83 min. MS (ESI) m/z 642 [M−H]⁻.

¹H NMR (400 MHZ, MeOH-d₄) δ 8.08 (d, J=5.6 Hz, 1H), 7.80 (d, J=8.9 Hz, 2H), 7.23-7.12 (m, 3H), 7.08 (d, J=8.7 Hz, 1H), 7.04-6.96 (m, 3H), 6.05 (q, J=6.5 Hz, 1H), 5.76 (s, 1H), 2.88 (d, J=16.9 Hz, 1H), 2.70-2.56 (m, 1H), 2.30 (d, J=13.6 Hz, 1H), 2.07-1.84 (m, 3H), 1.44 (d, J=6.5 Hz, 3H).

### Example 113

### 3-[[(7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]carbamoyl]bicyclo[1.1.1]pentane-1-carboxylic acid

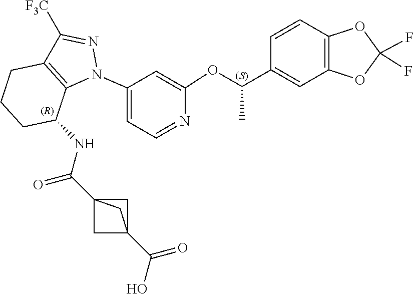

### Step 1. Synthesis of methyl 3-[[(7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]carbamoyl]bicyclo[1.1.1]pentane-1-carboxylate

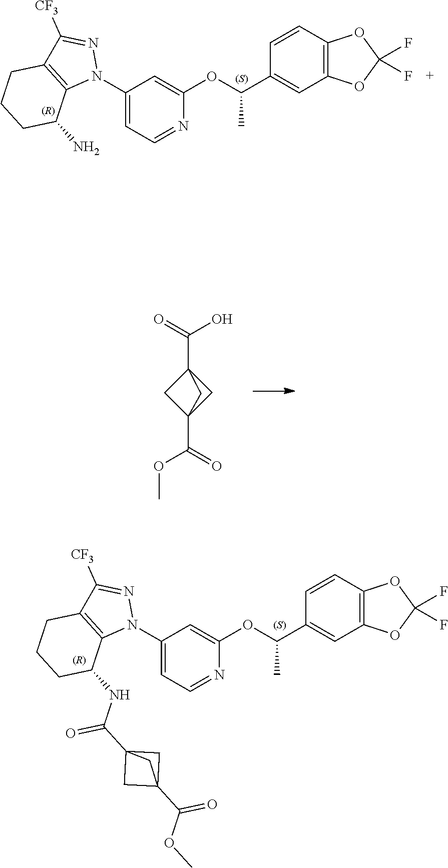

To a solution of (7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-amine (obtained similarly to Example 109, Step 1 to Step 3) (60 mg, 0.124 mmol), 3-methoxycarbonylbicyclo[1.1.1]pentane-1-carboxylic acid (25 mg, 0.149 mmol) and 4-dimethylaminopyridine (23 mg, 0.187 mmol) in DCM (3 mL) was added 1-(3-dimethylaminopropyl)-3-ethylcarbodiimide hydrochloride (36 mg, 0.187 mmol). The reaction was stirred at rt for 30 min. Then the mixture was diluted with water (20 mL) and extracted with EA (20 mL×3). The organic phase was washed with brine, dried over Na₂SO₄ and concentrated under vacuum. The residue was purified by flash column chromatography (PE:EA=5:1) to give methyl 3-[[(7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]carbamoyl]bicyclo[1.1.1]pentane-1-carboxylate (50 mg, 0.0788 mmol, 63.3% yield) as a white solid.

LCMS (Method 1): Retention time=2.21 min. MS (ESI) m/z 451.0 [M-184+H]⁺.

### Step 2. Synthesis of 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]amino]benzoic acid

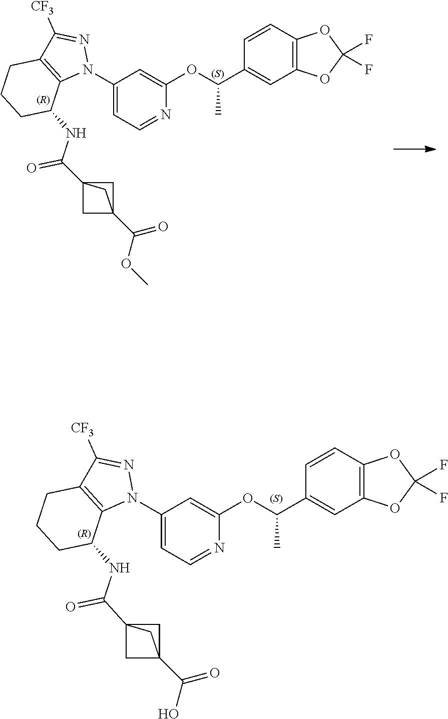

The title compounds were obtained by hydrolysis of the esters obtained above following the protocol of Step 7a and 7b of Example of 94.

LCMS (Method 2): Retention time=1.68 min. MS (ESI) m/z 619.0 [M−H]⁻.

Chiral Method: Column Name: (R,R) Whelk-O1 4.6*100 mm 5 μm Solvent: EtOH [0.2% NH₃ (7M in MeOH)]; Find at 1.390 min; ee: 100%

¹H NMR (400 MHZ, MeOH-d₄) δ 8.16 (d, J=5.6 Hz, 1H), 7.37 (s, 1H), 7.32-7.25 (m, 1H), 7.15 (d, J=8.3 Hz, 1H), 7.09 (dd, J=5.6, 1.7 Hz, 1H), 6.93 (d, J=1.4 Hz, 1H), 6.21 (q, J=6.5 Hz, 1H), 5.43 (t, J=5.8 Hz, 1H), 2.75-2.61 (m, 2H), 2.13-2.02 (m, 1H), 1.95-1.78 (m, 8H), 1.76-1.66 (m, 1H), 1.63 (d, J=6.5 Hz, 3H).

### Example 114

### 4-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]carbamoyl]benzoic acid

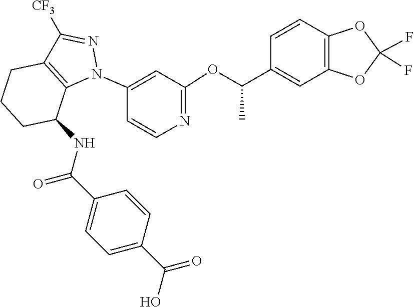 [SMILES: C[C@H](Oc1cc(-n2nc(C(F)(F)F)c3c2[C@@H](NC(=O)c2ccc(C(=O)O)cc2)CCC3)ccn1)c1ccc2c(c1)OC(F)(F)O2]

The compound Example 114 was synthesized similarly as Example 113 by using 4-methoxycarbonylbenzoic acid instead of 3-methoxycarbonylbicyclo[1.1.1]pentane-1-carboxylic acid.

LCMS (Method 2): Retention time=1.75 min. MS (ESI) m/z 629.0 [M−H]⁻.

Chiral Method: Column Name: OJ-H 4.6*100 mm 5 μm Solvent: MeOH [0.2% NH₃ (7M in MeOH)]; Find at 1.400 min; ee: 100%

¹H NMR (400 MHZ, MeOH-d₄) δ 8.09 (d, J=5.6 Hz, 1H), 7.93 (d, J=8.4 Hz, 2H), 7.50 (d, J=8.3 Hz, 2H), 7.25 (d, J=1.1 Hz, 1H), 7.19-7.06 (m, 3H), 7.01 (d, J=1.4 Hz, 1H), 6.06 (q, J=6.5 Hz, 1H), 5.65 (d, J=5.7 Hz, 1H), 2.83-2.65 (m, 2H), 2.24-2.11 (m, 1H), 2.05-1.83 (m, 3H), 1.47 (d, J=6.5 Hz, 3H).

### Example 115 (P1 and P2)

### 4-[[(7S)-1-[2-[(1S)-1-(2,2-difluoro-1,3-benzodioxol-5-yl)ethoxy]-4-pyridyl]-5-methyl-3-(trifluoromethyl)-6,7-dihydro-4H-pyrazolo[4,3-c]pyridine-7-yl]oxy]benzoic acid

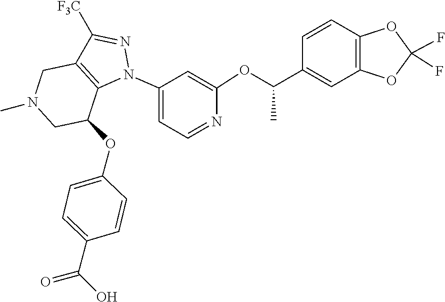 [SMILES: C[C@H](Oc1cc(-n2nc(C(F)(F)F)c3c2[C@@H](Oc2ccc(C(=O)O)cc2)CN(C)C3)ccn1)c1ccc2c(c1)OC(F)(F)O2]

and

### 4-[[(7R)-1-[2-[(1S)-1-(2,2-difluoro-1,3-benzodioxol-5-yl)ethoxy]-4-pyridyl]-5-methyl-3-(trifluoromethyl)-6,7-dihydro-4H-pyrazolo[4,3-c]pyridine-7-yl]oxy]benzoic acid

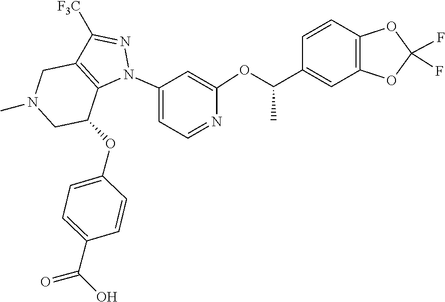 [SMILES: C[C@H](Oc1cc(-n2nc(C(F)(F)F)c3c2[C@H](Oc2ccc(C(=O)O)cc2)CN(C)C3)ccn1)c1ccc2c(c1)OC(F)(F)O2]

### Step 1. Synthesis of methyl 4-((1-(2-((S)-1-(2,2-difluorobenzo[d][1,3]dioxol-5-yl)ethoxy)pyridine-4-yl)-3-(trifluoromethyl)-4,5,6,7-tetrahydro-1H-pyrazolo[4,3-c]pyridine-7-yl)oxy)benzoate

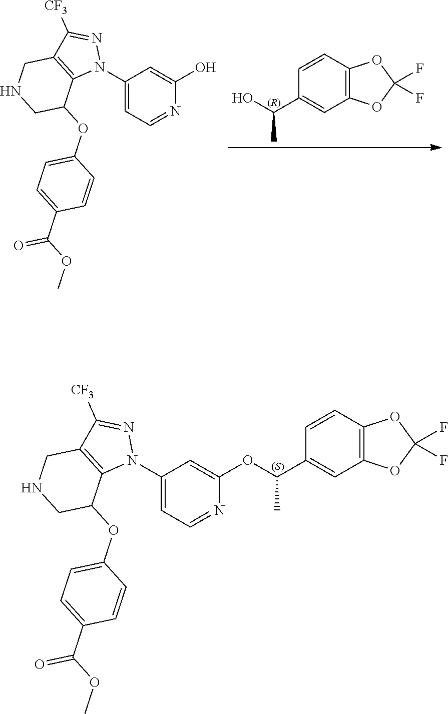

To a solution of methyl 4-[[1-(2-hydroxy-4-pyridyl)-3-(trifluoromethyl)-4,5,6,7-tetrahydropyrazolo[4,3-c]pyridine-7-yl]oxy]benzoate (Intermediate E1) (650 mg, 1.50 mmol) and (1R)-1-(2,2-difluoro-1,3-benzodioxol-5-yl)ethanol (Intermediate C-1R) (333 mg, 1.65 mmol) in THF (10 mL) were added triphenylphosphine (1177 mg, 4.49 mmol) and diethyl azodicarboxylate (0.36 mL, 2.24 mmol) at 0° C. The reaction was stirred at room temperature for 2 h. The reaction mixture was concentrated in vacuo to give a residue, which was purified by flash silica gel column chromatography (0% to 30% ethyl acetate in petroleum ether) to obtain (240 mg, 23.3% yield) as a yellow solid.

LCMS (Method 1): Retention time=2.35 min. MS (ESI) m/z 619.0 [M+H]⁺.

### Step 2. Synthesis of methyl 4-((1-(2-((S)-1-(2,2-difluorobenzo[d][1,3]dioxol-5-yl)ethoxy)pyridine-4-yl)-5-methyl-3-(trifluoromethyl)-4,5,6,7-tetrahydro-1H-pyrazolo[4,3-c]pyridine-7-yl)oxy)benzoate

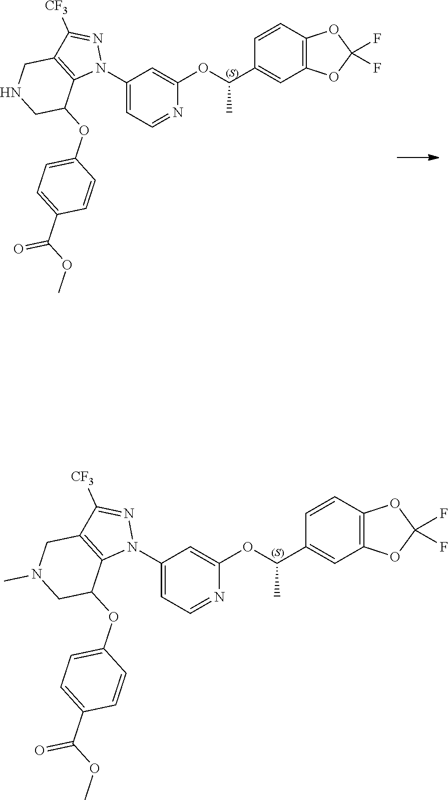

To a solution of methyl 4-[[1-[2-[(1S)-1-(2,2-difluoro-1,3-benzodioxol-5-yl)ethoxy]-4-pyridyl]-3-(trifluoromethyl)-4,5,6,7-tetrahydropyrazolo[4,3-c]pyridine-7-yl]oxy]benzoate (240 mg, 0.39 mmol) in THF (10 mL) was added potassium carbonate (161 mg, 1.16 mmol), followed by methyl iodide (0.049 mL, 0.78 mmol) at 0° C. The reaction was stirred at room temperature for 8 h. The reaction mixture was concentrated in vacuo to give a residue, which was purified by flash silica gel column chromatography (0% to 40% ethyl acetate in petroleum ether) to obtain the title compound (100 mg, 39.9% yield) as a colorless oil. LCMS (Method 1): Retention time=2.57 min. MS (ESI) m/z 633.2 [M+H]⁺.

### Step 3. Synthesis of 4-(((S)-1-(2-((S)-1-(2,2-difluorobenzo[d][1,3]dioxol-5-yl)ethoxy)pyridine-4-yl)-5-methyl-3-(trifluoromethyl)-4,5,6,7-tetrahydro-1H-pyrazolo[4,3-c]pyridine-7-yl)oxy)benzoic acid and 4-(((R)-1-(2-((S)-1-(2,2-difluorobenzo[d][1,3]dioxol-5-yl)ethoxy)pyridine-4-yl)-5-methyl-3-(trifluoromethyl)-4,5,6,7-tetrahydro-1H-pyrazolo[4,3-c]pyridine-7-yl)oxy)benzoic acid

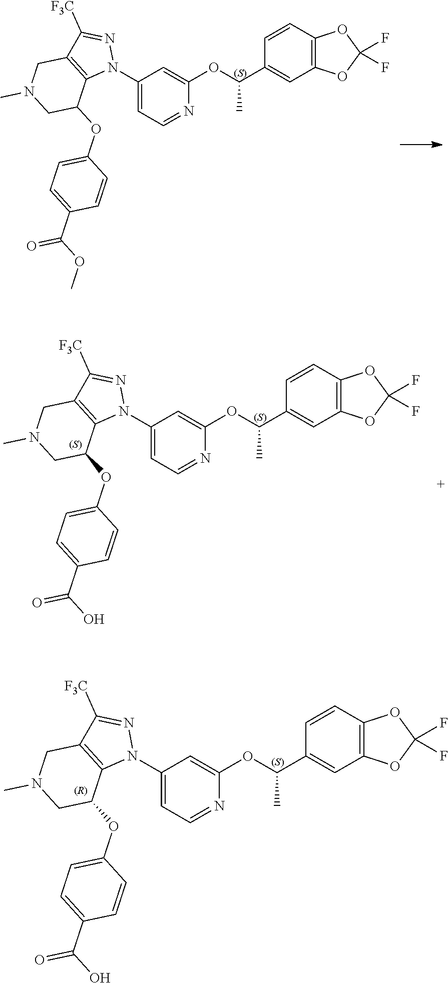

To a solution of methyl 4-[[1-[2-[(1S)-1-(2,2-difluoro-1,3-benzodioxol-5-yl)ethoxy]-4-pyridyl]-5-methyl-3-(trifluoromethyl)-6,7-dihydro-4H-pyrazolo[4,3-c]pyridine-7-yl]oxy]benzoate (100 mg, 0.16 mmol) in methanol (2 mL)/THF (2 mL)/water (1 mL) was added lithium hydroxide monohydrate (33 mg, 0.79 mmol). The reaction was stirred at 45° C. for 1 h. The reaction mixture was diluted with water (10 mL), the pH adjusted to ˜5 with citric acid, and then extracted with ethyl acetate (15 mL×3). The EA solution was washed with brine, dried (Na₂SO₄), filtered and concentrated under vacuum. The residue was purified by prep-HPLC (NH₄HCO₃ condition) to afford a racemic mixture which was then separated by SFC to obtain P1 (23 mg, 23.1% yield) as a white solid and P2 (30 mg, 30.2% yield) as a white solid.

### P1:

LCMS (Method 2): Retention time=1.76 min. MS (ESI) m/z 619.0 [M+H]⁺.

¹H NMR (400 MHZ, MeOH-d₄) δ 8.17 (d, J=5.5 Hz, 1H), 7.96 (d, J=8.8 Hz, 2H), 7.26 (s, 1H), 7.19 (d, J=8.7 Hz, 3H), 7.06 (dd, J=24.8, 6.7 Hz, 2H), 6.91 (s, 1H), 6.16 (q, J=6.3 Hz, 1H), 5.62 (s, 1H), 3.90 (d, J=15.2 Hz, 1H), 3.46 (d, J=15.2 Hz, 1H), 3.22-3.14 (m, 1H), 2.93 (dd, J=12.9, 3.4 Hz, 1H), 2.52 (s, 3H), 1.60 (d, J=6.5 Hz, 3H).

### P2:

LCMS (Method 2): Retention time=1.77 min. MS (ESI) m/z 619.0 [M+H]⁺.

¹H NMR (400 MHZ, MeOH-d₄) δ 8.17 (d, J=5.5 Hz, 1H), 7.95 (d, J=8.8 Hz, 2H), 7.27 (s, 1H), 7.23-7.14 (m, 3H), 7.10 (d, J=8.2 Hz, 1H), 7.03 (d, J=5.4 Hz, 1H), 6.91 (s, 1H), 6.17 (q, J=6.4 Hz, 1H), 5.62 (s, 1H), 3.90 (d, J=15.0 Hz, 1H), 3.47 (d, J=15.5 Hz, 1H), 3.21-3.16 (m, 1H), 2.93 (dd, J=12.8, 3.4 Hz, 1H), 2.52 (s, 3H), 1.60 (d, J=6.5 Hz, 3H).

### Example 116

### 4-[[1-[3-[1-(2,2-difluoro-1,3-benzodioxol-5-yl)ethylsulfanyl]phenyl]-3-(trifluoromethyl)-4,5,6,7-tetrahydroindazol-7-yl]oxy]benzoic acid

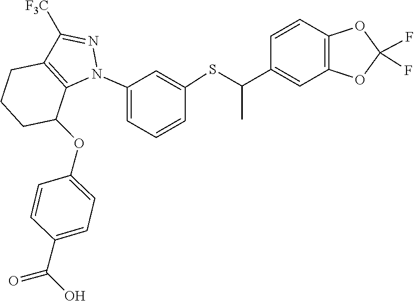 [SMILES: CC(Sc1cccc(-n2nc(C(F)(F)F)c3c2C(Oc2ccc(C(=O)O)cc2)CCC3)c1)c1ccc2c(c1)OC(F)(F)O2]

### Step 1. Synthesis of 5-(1-((3-bromophenyl)thio) ethyl)-2,2-difluorobenzo[d][1,3]dioxole

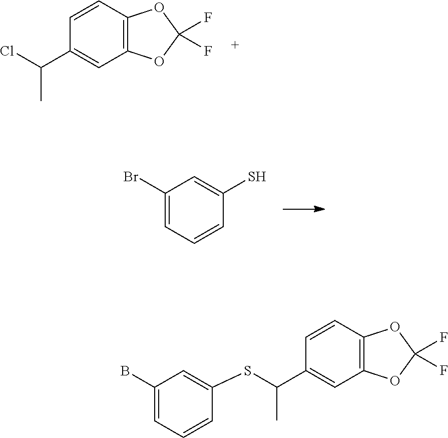

To a solution of 5-(1-chloroethyl)-2,2-difluoro-1,3-benzodioxole (Intermediate C-4) (1.00 eq, 1.80 g, 8.16 mmol) and 5-(1-chloroethyl)-2,2-difluoro-1,3-benzodioxole (1.00 eq, 1.80 g, 8.16 mmol) in acetonitrile (20 mL) were added potassium carbonate (5.00 eq, 5639 mg, 40.8 mmol) and potassium iodide (0.0500 eq, 68 mg, 0.408 mmol). The reaction was stirred at 66° C. for 16 h. The reaction mixture was filtered through a Celite pad, and the filtrate was concentrated. The residue was purified by flash column chromatography on silica gel (PE:EA=10:1) to give the crude product which was purified further by a reverse-phase flash chromatography (C18) (0.1% NH₄HCO₃ in water, 10-100% MeCN) to give 5-[1-(3-bromophenyl) sulfanylethyl]-2,2-difluoro-1,3-benzodioxole (2.40 g, 6.11 mmol, 74.8% yield) as a colorless oil.

LCMS (Method 3): Retention time=2.49 min. MS: not observed.

¹H NMR (400 MHZ, DMSO-d₆) δ 7.48 (s, 2H), 7.41 (ddd, J=7.9, 1.8, 1.0 Hz, 1H), 7.36-7.28 (m, 2H), 7.27-7.18 (m, 2H), 4.77 (q, J=6.9 Hz, 1H), 1.55 (d, J=7.0 Hz, 3H).

### Step 2. Synthesis of (3-((1-(2,2-difluorobenzo[d][1,3]dioxol-5-yl)ethyl)thio)phenyl)boronic acid

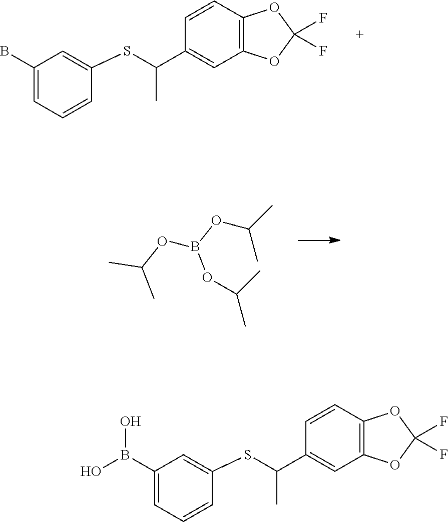

To a solution of 5-[1-(3-bromophenyl) sulfanylethyl]-2,2-difluoro-1,3-benzodioxole (1.00 eq, 1.00 g, 2.68 mmol) in dry THF (10 mL) was added n-butyllithium (1.00 eq, 1.1 mL, 2.68 mmol) at −78° C. The reaction mixture was stirred for 30 min. A solution of triisopropyl borate (1.50 eq, 0.93 mL, 4.02 mmol) in dry THF (5 mL) was added dropwise with stirring at −78° C. After addition, the reaction was stirred at this temperature for 30 min, then allowed to warm to room temperature over a 1 h period. Hydrochloric acid (2.00 eq, 2.7 mL, 5.36 mmol) was added with stirring and the resulting mixture was concentrated and purified by reverse-phase flash chromatography (C18) (0.1% TFA in water, 10-100% ACN) to give [3-[1-(2,2-difluoro-1,3-benzodioxol-5-yl)ethylsulfanyl]phenyl]boronic acid (480 mg, 1.35 mmol, 50.3% yield) as a white solid.

LCMS (Method 4): Retention time=1.96 min. MS (ESI) m/z 337.2 [M−H]⁻.

### Step 3. Synthesis of 1-(3-((1-(2,2-difluorobenzo[d][1,3]dioxol-5-yl)ethyl)thio)phenyl)-3-(trifluoromethyl)-1,4,5,6-tetrahydro-7H-indazol-7-one

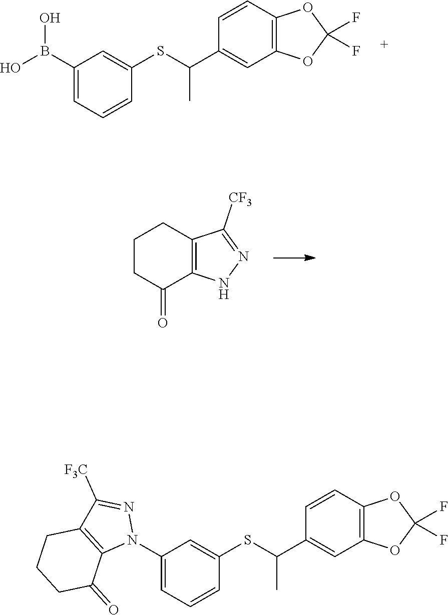 [SMILES: CC(Sc1cccc(B(O)O)c1)c1ccc2c(c1)OC(F)(F)O2 ; O=C1CCCc2c(C(F)(F)F)n[nH]c21 ; CC(Sc1cccc(-n2nc(C(F)(F)F)c3c2C(=O)CCC3)c1)c1ccc2c(c1)OC(F)(F)O2]

To a solution of [3-[1-(2,2-difluoro-1,3-benzodioxol-5-yl)ethylsulfanyl]phenyl]boronic acid (1.00 eq, 480 mg, 1.42 mmol) and 3-(trifluoromethyl)-1,4,5,6-tetrahydroindazol-7-one (Intermediate D-1) (1.00 eq, 290 mg, 1.42 mmol) in DCE (12 mL) were added triethylamine (1.00 eq, 0.20 mL, 1.42 mmol) and copper (II) acetate monohydrate (0.200 eq, 57 mg, 0.284 mmol). The mixture was then stirred at 60° C. for 24 h under air. The mixture was filtered, and the filtrate was concentrated under vacuum. The residue was purified by reverse-phase flash chromatography (C18) (0.1% NH₄HCO₃ in water, 10-100% MeCN) to give 1-[3-[1-(2,2-difluoro-1,3-benzodioxol-5-yl)ethylsulfanyl]phenyl]-3-(trifluoromethyl)-5,6-dihydro-4H-indazol-7-one (100 mg, 0.191 mmol, 13.4% yield) as a white solid.

LC-Mass (Method 4): Retention time=2.44 min. MS (ESI) m/z 497.1 [M+H]⁺.

### Step 4. Synthesis of 1-(3-((1-(2,2-difluorobenzo[d][1,3]dioxol-5-yl)ethyl)thio)phenyl)-3-(trifluoromethyl)-4,5,6,7-tetrahydro-1H-indazol-7-ol

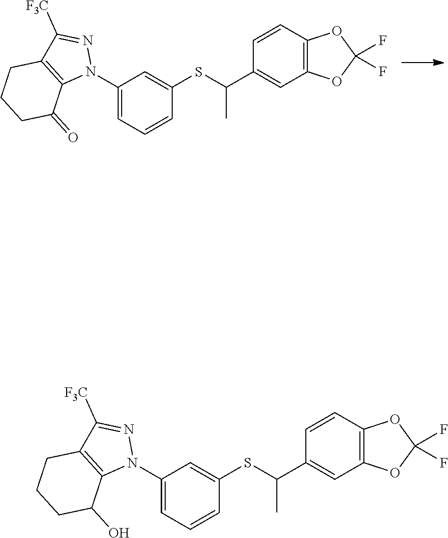 [SMILES: CC(Sc1cccc(-n2nc(C(F)(F)F)c3c2C(=O)CCC3)c1)c1ccc2c(c1)OC(F)(F)O2 ; CC(Sc1cccc(-n2nc(C(F)(F)F)c3c2C(O)CCC3)c1)c1ccc2c(c1)OC(F)(F)O2]

To a solution of 1-[3-[1-(2,2-difluoro-1,3-benzodioxol-5-yl)ethylsulfanyl]phenyl]-3-(trifluoromethyl)-5,6-dihydro-4H-indazol-7-one (1.00 eq, 100 mg, 0.201 mmol) in methanol (2 mL) was added sodium borohydride (1.20 eq, 9.1 mg, 0.242 mmol). The reaction was stirred at 0° C. for 1 h. The mixture was concentrated under vacuum, purified by reverse-phase flash chromatography (C18) (0.1% NH₄HCO₃ in water, 10-100% MeCN) to give 1-[3-[1-(2,2-difluoro-1,3-benzodioxol-5-yl)ethylsulfanyl]phenyl]-3-(trifluoromethyl)-4,5,6,7-tetrahydroindazol-7-ol (50 mg, 0.100 mmol, 49.80% yield) as a white solid.

LC-Mass (Method 4): Retention time=2.39 min. MS (ESI) m/z 499.1 [M+H]⁺.

¹H NMR (400 MHZ, DMSO-d₆) δ 7.81-7.74 (m, 1H), 7.66 (dddd, J=15.9, 8.0, 2.0, 1.1 Hz, 1H), 7.49 (t, J=1.5 Hz, 1H), 7.46 (t, J=7.9 Hz, 1H), 7.42-7.37 (m, 1H), 7.29 (dd, J=8.3, 1.7 Hz, 1H), 7.22 (dd, J=8.3, 1.5 Hz, 1H), 5.54 (s, 1H), 4.74 (qd, J=6.8, 2.2 Hz, 1H), 4.63 (dt, J=15.0 Hz, 1H), 2.74-2.64 (m, 1H), 2.49-2.42 (m, 1H), 1.98-1.86 (m, 2H), 1.80-1.66 (m, 2H), 1.58 (d, J=6.9 Hz, 3H).

### Step 5. Synthesis of methyl 4-((1-(3-((1-(2,2-difluorobenzo[d][1,3]dioxol-5-yl)ethyl)thio)phenyl)-3-(trifluoromethyl)-4,5,6,7-tetrahydro-1H-indazol-7-yl)oxy)benzoate

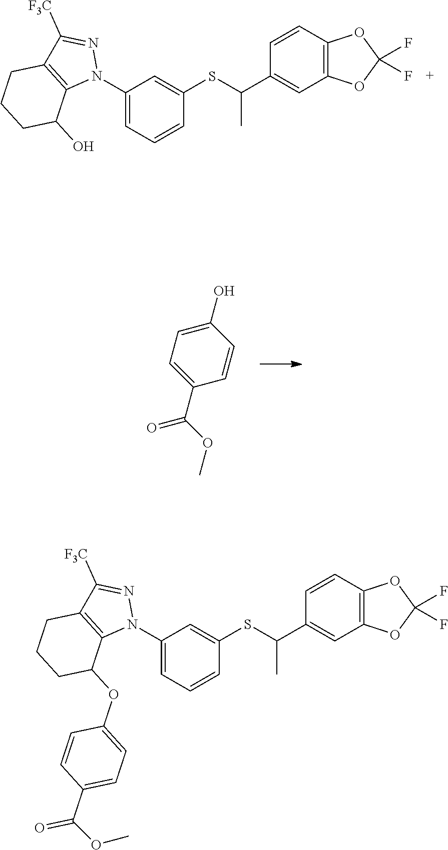

The title compound was obtained by following the procedure in Step 3, Example 36. LC-Mass (Method 4): Retention time=2.22 min. MS (ESI) m/z 633.1 [M+H]⁺.

### Step 6. Synthesis of 4-((1-(3-((1-(2,2-difluorobenzo[d][1,3]dioxol-5-yl)ethyl)thio)phenyl)-3-(trifluoromethyl)-4,5,6,7-tetrahydro-1H-indazol-7-yl)oxy)benzoic acid

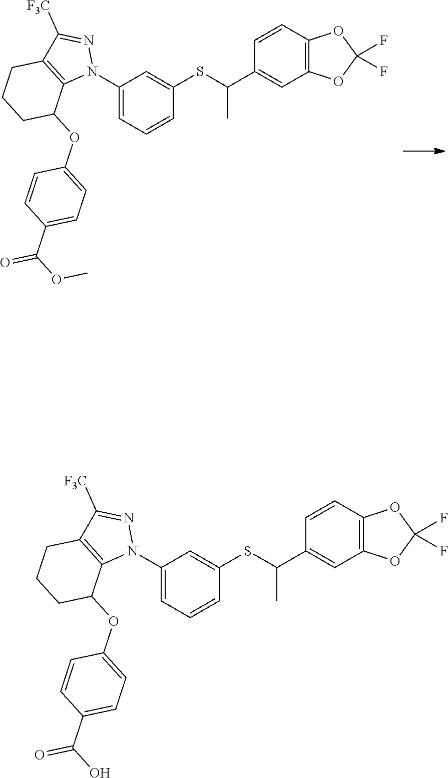 [SMILES: COC(=O)c1ccc(OC2CCCc3c(C(F)(F)F)nn(-c4cccc(SC(C)c5ccc6c(c5)OC(F)(F)O6)c4)c32)cc1 ; CC(Sc1cccc(-n2nc(C(F)(F)F)c3c2C(Oc2ccc(C(=O)O)cc2)CCC3)c1)c1ccc2c(c1)OC(F)(F)O2]

The title compound was obtained by following the procedure in Step 4, Example 36. LC-Mass (Method 4): Retention time=1.66 min. MS (ESI) m/z 619.2 [M+H]⁺.

¹H NMR (400 MHZ, MeOH-d₄) δ 7.98-7.88 (m, 2H), 7.59-7.50 (m, 1H), 7.46-7.37 (m, 1H), 7.29-7.08 (m, 3H), 6.91 (dddd, J=22.3, 10.0, 9.1, 5.0 Hz, 4H), 5.53 (dd, J=7.2, 3.0 Hz, 1H), 4.14 (dq, J=62.4, 7.0 Hz, 1H), 2.87 (ddd, J=3.4, 2.5, 1.3 Hz, 1H), 2.62 (ddd, J=11.3, 10.6, 5.8 Hz, 1H), 2.36-2.23 (m, 1H), 2.08-1.78 (m, 3H), 1.42 (dd, J=7.0, 1.3 Hz, 3H).

### Example 117

### 4-[[1-[3-[1-(2,2-difluoro-1,3-benzodioxol-5-yl)ethylsulfinyl]phenyl]-3-(trifluoromethyl)-4,5,6,7-tetrahydroindazol-7-yl]oxy]benzoic acid

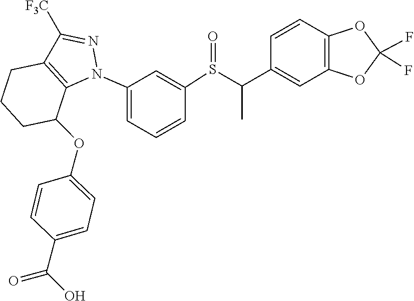 [SMILES: CC(c1ccc2c(c1)OC(F)(F)O2)S(=O)c1cccc(-n2nc(C(F)(F)F)c3c2C(Oc2ccc(C(=O)O)cc2)CCC3)c1]

### Step 1. Synthesis of 4-[[1-[3-[1-(2,2-difluoro-1,3-benzodioxol-5-yl)ethylsulfinyl]phenyl]-3-(trifluoromethyl)-4,5,6,7-tetrahydroindazol-7-yl]oxy]benzoic acid

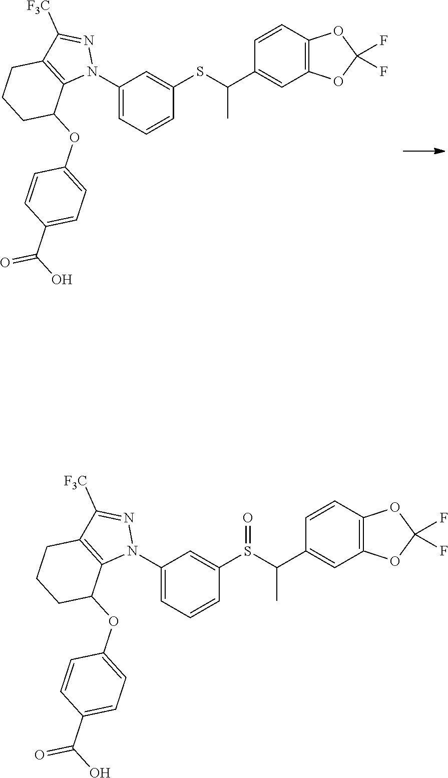 [SMILES: CC(Sc1cccc(-n2nc(C(F)(F)F)c3c2C(Oc2ccc(C(=O)O)cc2)CCC3)c1)c1ccc2c(c1)OC(F)(F)O2 ; CC(c1ccc2c(c1)OC(F)(F)O2)S(=O)c1cccc(-n2nc(C(F)(F)F)c3c2C(Oc2ccc(C(=O)O)cc2)CCC3)c1]

To a solution of 4-[[1-[3-[1-(2,2-difluoro-1,3-benzodioxol-5-yl)ethylsulfanyl]phenyl]-3-(trifluoromethyl)-4,5,6,7-tetrahydroindazol-7-yl]oxy]benzoic acid (1.00 eq, 28 mg, 0.0453 mmol) in DCM (2.5 mL) was added 3-chloroperoxybenzoic acid (1.00 eq, 7.8 mg, 0.0453 mmol). The reaction was stirred at rt for 20 min, and reaction progress was monitored by LCMS. After reaction completion, the reaction mixture was concentrated and purified by reverse-phase flash chromatography (C18) (0.1% NH₄HCO₃ in water, 10-100% acetonitrile) to give 4-[[1-[3-[1-(2,2-difluoro-1,3-benzodioxol-5-yl)ethylsulfinyl]phenyl]-3-(trifluoromethyl)-4,5,6,7-tetrahydroindazol-7-yl]oxy]benzoic acid (22 mg, 0.0347 mmol, 76.6% yield) as a white solid.

LC-Mass (Method 4): Retention time=1.80 min. MS (ESI) m/z 635.1 [M+H]⁺.

¹H NMR (400 MHZ, MeOH-d₄) δ 8.03-7.90 (m, 2H), 7.76-7.38 (m, 3H), 7.32-6.48 (m, 6H), 5.73 (ddd, J=17.7, 8.6, 5.5 Hz, 1H), 3.72 (ddq, J=60.6, 20.9, 7.2 Hz, 1H), 2.94-2.83 (m, 1H), 2.65 (ddd, J=16.7, 10.4, 5.9 Hz, 1H), 2.37-2.21 (m, 1H), 2.05-1.87 (m, 3H), 1.60-1.35 (m, 3H).

### Example 118

### 4-[[1-[3-[1-(2,2-difluoro-1,3-benzodioxol-5-yl)ethylsulfonyl]phenyl]-3-(trifluoromethyl)-4,5,6,7-tetrahydroindazol-7-yl]oxy]benzoic acid

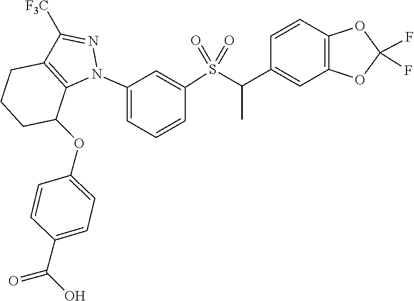 [SMILES: CC(c1ccc2c(c1)OC(F)(F)O2)S(=O)(=O)c1cccc(-n2nc(C(F)(F)F)c3c2C(Oc2ccc(C(=O)O)cc2)CCC3)c1]

### Step 1. Synthesis of 4-[[1-[3-[1-(2,2-difluoro-1,3-benzodioxol-5-yl)ethylsulfonyl]phenyl]-3-(trifluoromethyl)-4,5,6,7-tetrahydroindazol-7-yl]oxy]benzoic acid

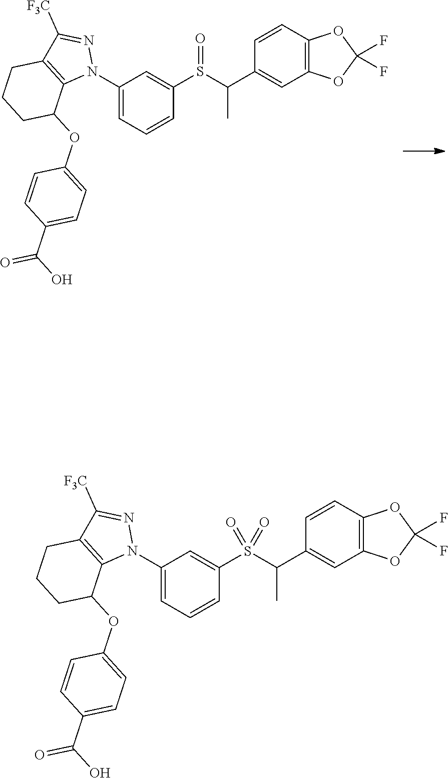 [SMILES: CC(c1ccc2c(c1)OC(F)(F)O2)S(=O)c1cccc(-n2nc(C(F)(F)F)c3c2C(Oc2ccc(C(=O)O)cc2)CCC3)c1 ; CC(c1ccc2c(c1)OC(F)(F)O2)S(=O)(=O)c1cccc(-n2nc(C(F)(F)F)c3c2C(Oc2ccc(C(=O)O)cc2)CCC3)c1]

To a solution of 4-[[1-[3-[1-(2,2-difluoro-1,3-benzodioxol-5-yl)ethylsulfinyl]phenyl]-3-(trifluoromethyl)-4,5,6,7-tetrahydroindazol-7-yl]oxy]benzoic acid (1.00 eq, 14 mg, 0.0221 mmol) in DCM (2 mL) was added 3-chloroperoxybenzoic acid (1.00 eq, 3.8 mg, 0.0221 mmol). The reaction was stirred at rt for 20 min. The reaction mixture was concentrated and purified by reverse-phase flash chromatography (C18) (0.1% NH₄HCO₃ in water, 10-100% acetonitrile) to give 4-[[1-[3-[1-(2,2-difluoro-1,3-benzodioxol-5-yl)ethylsulfonyl]phenyl]-3-(trifluoromethyl)-4,5,6,7-tetrahydroindazol-7-yl]oxy]benzoic acid (8.0 mg, 0.0123 mmol, 55.7% yield) as a white solid.

LC-Mass (Method 3): Retention time=2.23 min. MS (ESI) m/z 651.2 [M+H]⁺.

¹H NMR (400 MHZ, MeOH-d₄) δ 8.04-7.85 (m, 4H), 7.62-7.42 (m, 2H), 7.12-6.94 (m, 4H), 6.79 (ddd, J=12.6, 8.4, 1.7 Hz, 1H), 5.75 (dt, J=27.8, 2.8 Hz, 1H), 4.05 (dq, J=130.9, 7.1 Hz, 1H), 2.89 (dd, J=19.0, 2.4 Hz, 1H), 2.65 (ddd, J=11.1, 9.1, 5.3 Hz, 1H), 2.38-2.22 (m, 1H), 2.05-1.87 (m, 3H), 1.51 (dd, J=28.1, 7.1 Hz, 3H).

### Example 119

### 4-[[1-[2-[3-[1-(2,2-difluoro-1,3-benzodioxol-5-yl)ethyl]oxetan-3-yl]-4-pyridyl]-3-(trifluoromethyl)-4,5,6,7-tetrahydroindazol-7-yl]oxy]benzoic

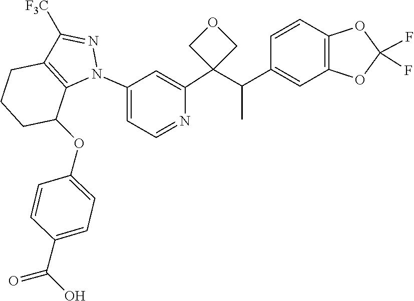 [SMILES: CC(c1ccc2c(c1)OC(F)(F)O2)C1(c2cc(-n3nc(C(F)(F)F)c4c3C(Oc3ccc(C(=O)O)cc3)CCC4)ccn2)COC1]

### Step 1. Synthesis of dimethyl 2-(4-bromo-2-pyridyl)propanedioate

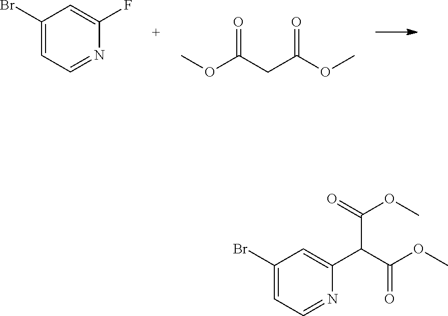

To a solution of 4-bromo-2-fluoro-pyridine (18.00 g, 102 mmol) and dimethyl malonate (12 mL, 102 mmol) in DMSO (120 mL) was added cesium carbonate (99.97 g, 307 mmol). The reaction was stirred at 100° C. for 4 h. The suspension was filtered through a Celite pad, and the filter cake was rinsed with EA (100 mL×3). The filtrate was washed with water (100 mL×2). The organic layers were combined and concentrated. The residue was purified by reversed-phase flash chromatography (C18) (0.1% NH₄HCO₃ in water, 10-100% acetonitrile) to give dimethyl 2-(4-bromo-2-pyridyl)propanedioate (6.80 g, 23.6 mmol, 23.1% yield) as a white solid.

LCMS (Method 4): Retention time=1.51 min. MS (ESI) m/z 288.0, 290.0 [M+H]⁺.

### Step 2. Synthesis of dimethyl 2-(4-bromo-2-pyridyl)-2-[1-(2,2-difluoro-1,3-benzodioxol-5-yl)ethyl]propanedioate

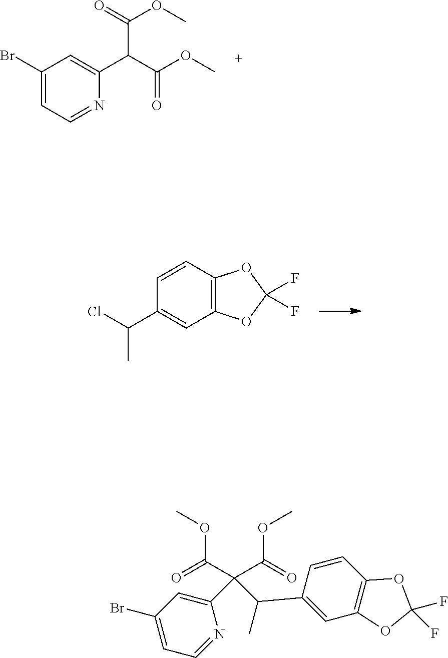 [SMILES: COC(=O)C(C(=O)OC)c1cc(Br)ccn1 ; CC(Cl)c1ccc2c(c1)OC(F)(F)O2 ; COC(=O)C(C(=O)OC)(c1cc(Br)ccn1)C(C)c1ccc2c(c1)OC(F)(F)O2]

To a solution of dimethyl 2-(4-bromo-2-pyridyl)propanedioate (4.50 g, 15.6 mmol) and 5-(1-chloroethyl)-2,2-difluoro-1,3-benzodioxole (4.13 g, 18.7 mmol) in DMF (20 mL) was added potassium carbonate (6.48 g, 46.9 mmol) and sodium bromide (161 mg, 1.56 mmol). The reaction was stirred at 60° C. for 16 h. The reaction mixture was concentrated and purified by reverse-phase flash chromatography (C18) (0.1% NH₄HCO₃ in water, 10-100% acetonitrile) to give dimethyl 2-(4-bromo-2-pyridyl)-2-[1-(2,2-difluoro-1,3-benzodioxol-5-yl)ethyl]propanedioate (6.50 g, 13.8 mmol, 88.1% yield) as a colorless oil.

LCMS (Method 4): Retention time=2.22 min. MS (ESI) m/z 472.0, 474.0 [M+H]⁺.

### Step 3. Synthesis of 2-(4-bromo-2-pyridyl)-2-[1-(2,2-difluoro-1,3-benzodioxol-5-yl)ethyl]propane-1,3-diol

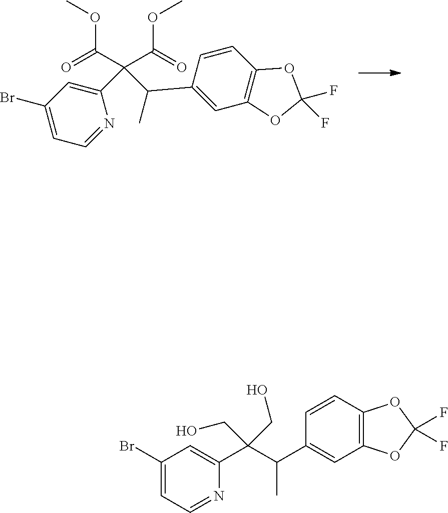 [SMILES: COC(=O)C(C(=O)OC)(c1cc(Br)ccn1)C(C)c1ccc2c(c1)OC(F)(F)O2 ; CC(c1ccc2c(c1)OC(F)(F)O2)C(CO)(CO)c1cc(Br)ccn1]

To the solution of dimethyl 2-(4-bromo-2-pyridyl)-2-[1-(2,2-difluoro-1,3-benzodioxol-5-yl)ethyl]propanedioate (4.50 g, 9.53 mmol) in DCM (30 mL) was added diisobutylaluminum hydride (1.36 g, 9.53 mmol) at −78° C. The mixture was stirred at −78° C. for 30 min. The reaction was quenched with saturated Rochelle salt aqueous solution. The mixture was stirred at room temperature for 30 min. and extracted with ethyl acetate (30 mL×3). The organic phases were combined and washed with brine, dried over Na₂SO₄, and concentrated under vacuum. The residue was purified by flash column chromatography (PE:EA=1:1) to give 2-(4-bromo-2-pyridyl)-2-[1-(2,2-difluoro-1,3-benzodioxol-5-yl)ethyl]propane-1,3-diol (1.80 g, 4.32 mmol, 45.3% yield) as a colorless oil.

LCMS (Method 4): Retention time=1.86 min. MS (ESI) m/z 416.0 [M+H]⁺.

### Step 4. Synthesis of 4-bromo-2-[3-[1-(2,2-difluoro-1,3-benzodioxol-5-yl)ethyl]oxetan-3-yl]pyridine

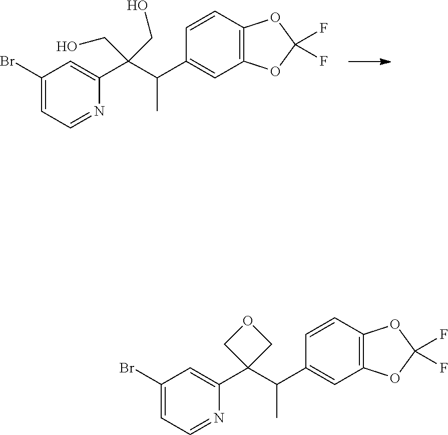 [SMILES: CC(c1ccc2c(c1)OC(F)(F)O2)C(CO)(CO)c1cc(Br)ccn1 ; CC(c1ccc2c(c1)OC(F)(F)O2)C1(c2cc(Br)ccn2)COC1]

To the solution of 2-(4-bromo-2-pyridyl)-2-[1-(2,2-difluoro-1,3-benzodioxol-5-yl)ethyl]propane-1,3-diol (450 mg, 1.08 mmol) in dry toluene (8 mL) were added zinc dimethyldithiocarbamate (CAS #: 137-30-4) (1323 mg, 4.32 mmol) and triphenylphosphine (296 mg, 1.13 mmol). The reaction was stirred at rt under a nitrogen atmosphere. To this mixture was added dropwise diethyl azodicarboxylate (0.18 mL, 1.13 mmol) over a period of 10 min, and stirred at 40° C. for 8 h. The suspension was filtered through a Celite pad and the filter cake was washed with EA. The filtrate was washed with water (50 mL), the organic layer was concentrated and purified by reversed-phase flash chromatography (C18) (0.1% NH₄HCO₃, 10-100% acetonitrile) to give 4-bromo-2-[3-[1-(2,2-difluoro-1,3-benzodioxol-5-yl)ethyl]oxetan-3-yl]pyridine (350 mg, 0.879 mmol, 81.3% yield) as a white solid.

LCMS (Method 1): Retention time=2.13 min. MS (ESI) m/z 397.9, 399.9 [M+H]⁺.

### Step 5. Synthesis of [2-[3-[1-(2,2-difluoro-1,3-benzodioxol-5-yl)ethyl]oxetan-3-yl]-4-pyridyl]boronic acid

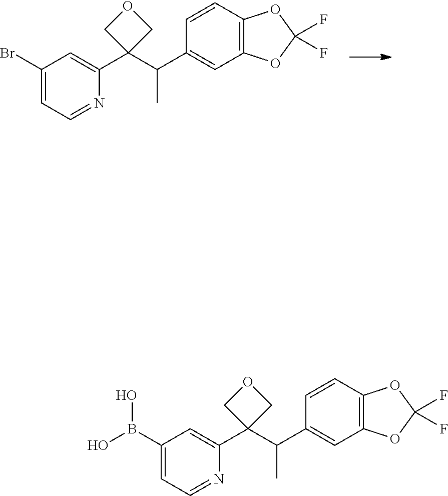 [SMILES: CC(c1ccc2c(c1)OC(F)(F)O2)C1(c2cc(Br)ccn2)COC1 ; CC(c1ccc2c(c1)OC(F)(F)O2)C1(c2cc(B(O)O)ccn2)COC1]

To a solution of 4-bromo-2-[3-[1-(2,2-difluoro-1,3-benzodioxol-5-yl)ethyl]oxetan-3-yl]pyridine (300 mg, 0.753 mmol) in 1,4-dioxane (12 mL) was added bis(pinacolato)diboron (1.20 eq, 230 mg, 0.904 mmol), potassium acetate (222 mg, 2.26 mmol) and 1,1′-bis(diphenylphosphino) ferrocene-palladium (II) dichloride dichloromethane complex (Pd(dppf)Cl₂·CH₂Cl₂)) (62 mg, 0.0753 mmol). The reaction was stirred at 80° C. for 2 h under N₂. The mixture was then concentrated and purified by reverse-phase flash chromatography (C18) (0.1% NH₄HCO₃ in water, 10-100% MeOH) to give [2-[3-[1-(2,2-difluoro-1,3-benzodioxol-5-yl)ethyl]oxetan-3-yl]-4-pyridyl]boronic acid (230 mg, 0.633 mmol, 84.0% yield) as a white solid.

LCMS (Method 2): Retention time=1.58 min. MS (ESI) m/z 362.0 [M+H]⁺.

### Step 6. Synthesis of 1-[2-[3-[1-(2,2-difluoro-1,3-benzodioxol-5-yl)ethyl]oxetan-3-yl]-4-pyridyl]-3-(trifluoromethyl)-5,6-dihydro-4H-indazol-7-one

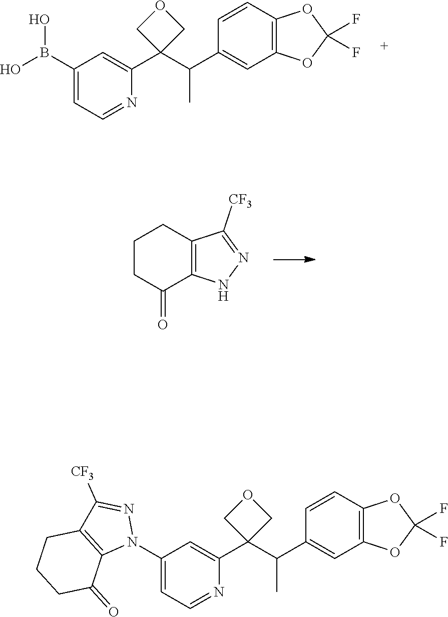 [SMILES: CC(c1ccc2c(c1)OC(F)(F)O2)C1(c2cc(B(O)O)ccn2)COC1 ; O=C1CCCc2c(C(F)(F)F)n[nH]c21 ; CC(c1ccc2c(c1)OC(F)(F)O2)C1(c2cc(-n3nc(C(F)(F)F)c4c3C(=O)CCC4)ccn2)COC1]

To a solution of [2-[3-[1-(2,2-difluoro-1,3-benzodioxol-5-yl)ethyl]oxetan-3-yl]-4-pyridyl]boronic acid (230 mg, 0.633 mmol) and 3-(trifluoromethyl)-1,4,5,6-tetrahydroindazol-7-one (129 mg, 0.633 mmol) in DCM (15 mL) were added triethylamine (0.088 mL, 0.633 mmol) and copper (II) acetate monohydrate (63 mg, 0.317 mmol). The mixture was then stirred at rt for 24 h under oxygen. The mixture was filtered and the filtrate was concentrated under vacuum, purified by reversed-phase flash chromatography (C18) (0.1% NH₄HCO₃ in water, 10-100% acetonitrile) to give 1-[2-[3-[1-(2,2-difluoro-1,3-benzodioxol-5-yl)ethyl]oxetan-3-yl]-4-pyridyl]-3-(trifluoromethyl)-5,6-dihydro-4H-indazol-7-one (120 mg, 0.230 mmol, 36.3% yield) as a white solid.

LCMS (Method 1): Retention time=2.20 min. MS (ESI) m/z 522.0 [M+H]⁺.

### Step 7. Synthesis of 1-[2-[3-[1-(2,2-difluoro-1,3-benzodioxol-5-yl)ethyl]oxetan-3-yl]-4-pyridyl]-3-(trifluoromethyl)-4,5,6,7-tetrahydroindazol-7-ol

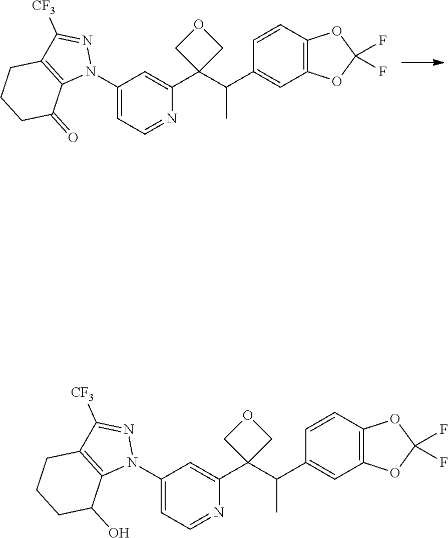 [SMILES: CC(c1ccc2c(c1)OC(F)(F)O2)C1(c2cc(-n3nc(C(F)(F)F)c4c3C(=O)CCC4)ccn2)COC1 ; CC(c1ccc2c(c1)OC(F)(F)O2)C1(c2cc(-n3nc(C(F)(F)F)c4c3C(O)CCC4)ccn2)COC1]

To a solution of 1-[2-[3-[1-(2,2-difluoro-1,3-benzodioxol-5-yl)ethyl]oxetan-3-yl]-4-pyridyl]-3-(trifluoromethyl)-5,6-dihydro-4H-indazol-7-one (70 mg, 0.134 mmol) in methanol (2 mL) was added sodium borohydride (6.1 mg, 0.161 mmol) at 0° C. and the mixture was stirred at 0° C. for 5 min. The mixture was then concentrated and the residue was purified by reversed-phase flash chromatography (C18) (0.1% NH₄HCO₃ in water, 10-100% acetonitrile) to give 1-[2-[3-[1-(2,2-difluoro-1,3-benzodioxol-5-yl)ethyl]oxetan-3-yl]-4-pyridyl]-3-(trifluoromethyl)-4,5,6,7-tetrahydroindazol-7-ol (60 mg, 0.115 mmol, 85.3% yield) as a white solid.

LCMS (Method 1): Retention time=2.14 min. MS (ESI) m/z 524.1 [M+H]⁺.

### Step 8. Synthesis of methyl 4-[[1-[2-[3-[1-(2,2-difluoro-1,3-benzodioxol-5-yl)ethyl]oxetan-3-yl]-4-pyridyl]-3-(trifluoromethyl)-4,5,6,7-tetrahydroindazol-7-yl]oxy]benzoate

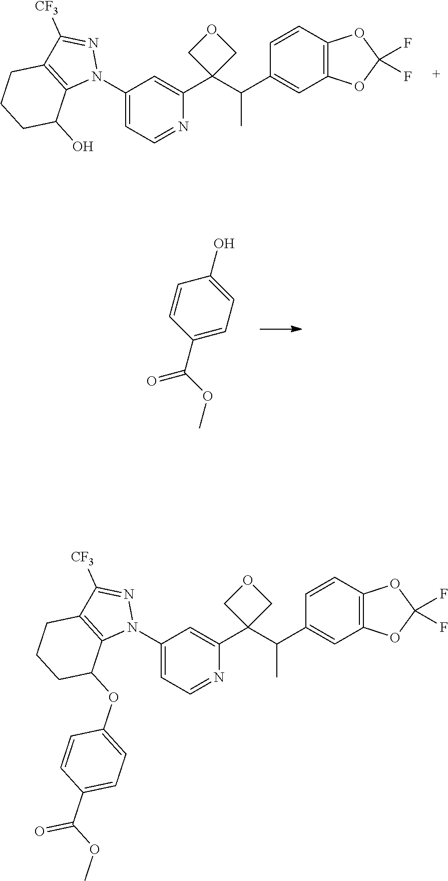 [SMILES: CC(c1ccc2c(c1)OC(F)(F)O2)C1(c2cc(-n3nc(C(F)(F)F)c4c3C(O)CCC4)ccn2)COC1 ; COC(=O)c1ccc(O)cc1 ; COC(=O)c1ccc(OC2CCCc3c(C(F)(F)F)nn(-c4ccnc(C5(C(C)c6ccc7c(c6)OC(F)(F)O7)COC5)c4)c32)cc1]

The title compound was obtained by following the procedure in Step3, Example 36. LCMS (Method 1): Retention time=2.38 min. MS (ESI) m/z 658.1 [M+H]⁺.

### Step 9. Synthesis of 4-[[1-[2-[3-[1-(2,2-difluoro-1,3-benzodioxol-5-yl)ethyl]oxetan-3-yl]-4-pyridyl]-3-(trifluoromethyl)-4,5,6,7-tetrahydroindazol-7-yl]oxy]benzoic acid

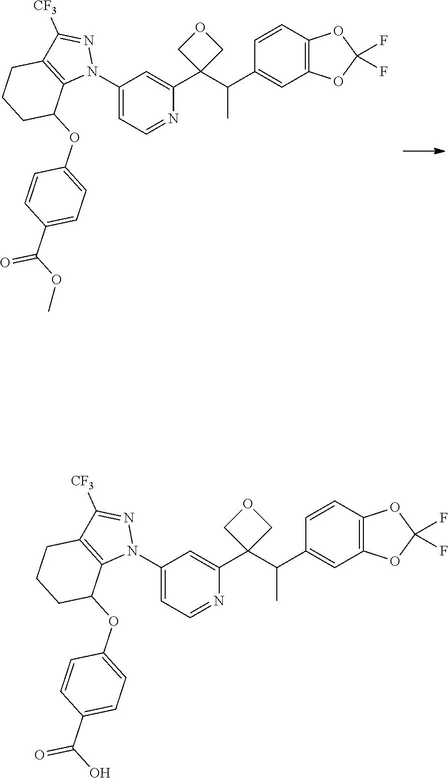 [SMILES: COC(=O)c1ccc(OC2CCCc3c(C(F)(F)F)nn(-c4ccnc(C5(C(C)c6ccc7c(c6)OC(F)(F)O7)COC5)c4)c32)cc1 ; CC(c1ccc2c(c1)OC(F)(F)O2)C1(c2cc(-n3nc(C(F)(F)F)c4c3C(Oc3ccc(C(=O)O)cc3)CCC4)ccn2)COC1]

The title compound was obtained by following the procedures in Step3, Example 36.

LCMS (Method 1): Retention time=1.72 min. MS (ESI) m/z 644.1 [M+H]⁺.

¹H NMR (400 MHZ, MeOH-d₄) δ 8.50 (dd, J=8.7, 5.5 Hz, 1H), 8.04 (dd, J=12.0, 8.9 Hz, 2H), 7.61 (dd, J=5.6, 2.0 Hz, 1H), 7.14-6.89 (m, 4H), 6.69 (dd, J=18.0, 1.5 Hz, 1H), 6.56 (dd, J=16.3, 8.3 Hz, 1H), 5.77 (d, J=9.0 Hz, 1H), 4.79-4.40 (m, 4H), 3.40-3.15 (m, 1H), 2.88 (d, J=17.0 Hz, 1H), 2.72-2.56 (m, 1H), 2.47-2.30 (m, 1H), 2.07-1.78 (m, 3H), 1.21 (dd, J=11.0, 7.2 Hz, 4H).

### Example 120 (P1 and P2)

### 1-[(7S)-1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylic acid

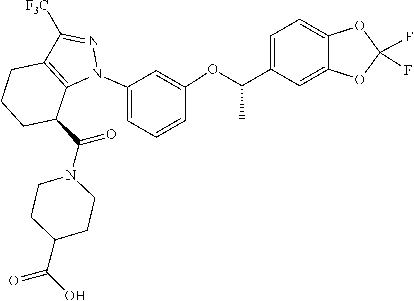 [SMILES: C[C@H](Oc1cccc(-n2nc(C(F)(F)F)c3c2[C@@H](C(=O)N2CCC(C(=O)O)CC2)CCC3)c1)c1ccc2c(c1)OC(F)(F)O2]

and

### 1-[(7R)-1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylic acid

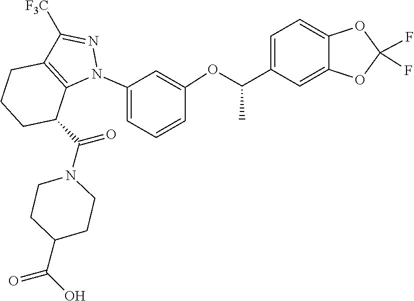 [SMILES: C[C@H](Oc1cccc(-n2nc(C(F)(F)F)c3c2[C@H](C(=O)N2CCC(C(=O)O)CC2)CCC3)c1)c1ccc2c(c1)OC(F)(F)O2]

### Step 1. Synthesis of 1-(3-bromophenyl)-7-chloro-3-(trifluoromethyl)-4,5,6,7-tetrahydroindazole

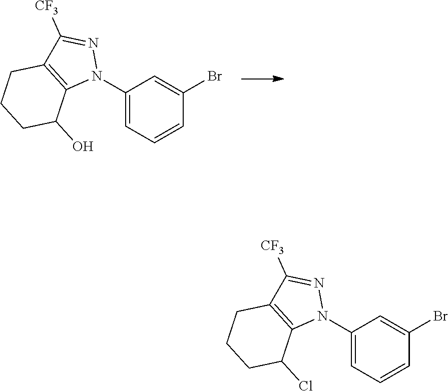 [SMILES: OC1CCCc2c(C(F)(F)F)nn(-c3cccc(Br)c3)c21 ; FC(F)(F)c1nn(-c2cccc(Br)c2)c2c1CCCC2Cl]

To a solution of 1-(3-bromophenyl)-3-(trifluoromethyl)-4,5,6,7-tetrahydroindazol-7-ol (Intermediate D-2b) (1.60 g, 4.43 mmol) and triethylamine (1.9 mL, 13.3 mmol) in DCM (20 mL) was added methanesulfonyl chloride (0.69 mL, 8.86 mmol) at 0° C. The reaction was stirred at rt for 4 h. The mixture was then diluted with water (20 mL) and extracted with DCM (20 mL×3). The organic phases were combined and washed with brine, dried over Na₂SO₄, and concentrated under vacuum. The residue was purified by flash column chromatography on silica gel (PE:EA=5:1) to give 1-(3-bromophenyl)-7-chloro-3-(trifluoromethyl)-4,5,6,7-tetrahydroindazole (1.60 g, 95.1%) as a colorless oil. LCMS (Method 3): Retention time=2.40 min. MS (ESI) m/z 379.0 [M+H]⁺.

### Step 3. Synthesis of 7-bromo-1-(3-bromophenyl)-3-(trifluoromethyl)-4,5,6,7-tetrahydroindazole

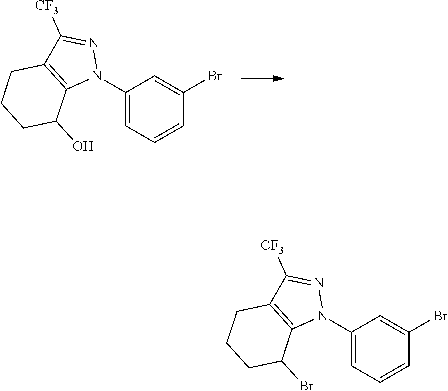 [SMILES: OC1CCCc2c(C(F)(F)F)nn(-c3cccc(Br)c3)c21 ; FC(F)(F)c1nn(-c2cccc(Br)c2)c2c1CCCC2Br]

To a solution of 1-(3-bromophenyl)-3-(trifluoromethyl)-4,5,6,7-tetrahydroindazol-7-ol (2.00 g, 5.54 mmol) and in DCM (20 mL) was added phosphorus tribromide (0.78 mL, 8.31 mmol) dropwise at 0° C. The reaction was stirred at 0° C. for 1.5 h. The mixture was then diluted with saturated NaHCO₃ (30 mL) and extracted with DCM (30 mL×2). The organic phase was washed with brine, dried over Na₂SO₄, and concentrated under vacuum. The residue was purified by flash column chromatography on silica gel (PE:EA=5:1) to give 7-bromo-1-(3-bromophenyl)-3-(trifluoromethyl)-4,5,6,7-tetrahydroindazole (1.50 g, 63.8%) as a yellow oil.

LCMS (Method 3): Retention time=2.42 min. MS (ESI) m/z 423.0 [M+H]⁺.

### Step 4. Synthesis of 1-(3-bromophenyl)-3-(trifluoromethyl)-4,5,6,7-tetrahydroindazole-7-carbonitrile

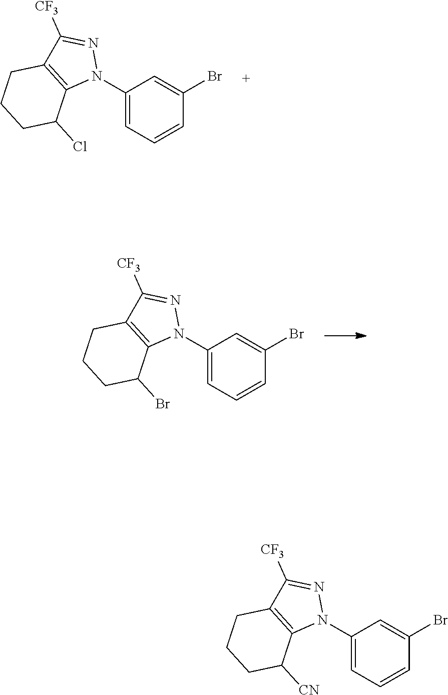 [SMILES: FC(F)(F)c1nn(-c2cccc(Br)c2)c2c1CCCC2Cl ; FC(F)(F)c1nn(-c2cccc(Br)c2)c2c1CCCC2Br ; N#CC1CCCc2c(C(F)(F)F)nn(-c3cccc(Br)c3)c21]

To a solution of 1-(3-bromophenyl)-7-chloro-3-(trifluoromethyl)-4,5,6,7-tetrahydroindazole (1.60 g, 4.21 mmol) and 7-bromo-1-(3-bromophenyl)-3-(trifluoromethyl)-4,5,6,7-tetrahydroindazole (1.50 g, 3.54 mmol) in DMF (15 mL) was added sodium cyanide (496 mg, 10.1 mmol). The reaction was stirred at 80° C. for 3 h. Then the mixture was diluted with water (30 mL) and extracted with EA (30 mL×3). The organic phase was washed with brine, dried over Na₂SO₄, and concentrated under vacuum. The residue was purified by flash column chromatography on silica gel (PE:EA=5:1) to give 1-(3-bromophenyl)-3-(trifluoromethyl)-4,5,6,7-tetrahydroindazole-7-carbonitrile (2.80 g, 92.7% yield) as a yellow solid.

LCMS (Method 3): Retention time=2.10 min. MS (ESI) m/z 370.1 [M+H]⁺.

### Step 5. Synthesis of 1-(3-bromophenyl)-3-(trifluoromethyl)-4,5,6,7-tetrahydroindazole-7-carboxylic acid

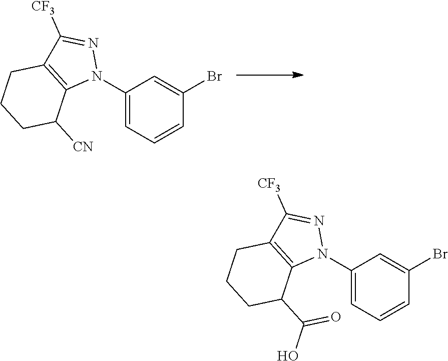 [SMILES: N#CC1CCCc2c(C(F)(F)F)nn(-c3cccc(Br)c3)c21 ; O=C(O)C1CCCc2c(C(F)(F)F)nn(-c3cccc(Br)c3)c21]

To a solution of 1-(3-bromophenyl)-3-(trifluoromethyl)-4,5,6,7-tetrahydroindazole-7-carbonitrile (1.00 g, 2.70 mmol) in acetic acid (4 mL) was added conc. Hydrochloric acid (8.0 mL, 96.0 mmol). The reaction mixture was stirred at 100° C. for 16 h. The reaction mixture was concentrated and purified by reversed-phase flash chromatography (C18) (0.1% NH₄HCO₃ in water, 10-100% acetonitrile) to give 1-(3-bromophenyl)-3-(trifluoromethyl)-4,5,6,7-tetrahydroindazole-7-carboxylic acid (910 mg, 86.6%) as a white solid.

LCMS (Method 1): Retention time=2.05 min. MS (ESI) m/z 388.9, 390.9 [M+H]⁺.

### Step 6. Synthesis of 1-[1-(3-bromophenyl)-3-(trifluoromethyl)-4,5,6,7-tetrahydroindazole-7-carbonyl]piperidine-4-carboxylate

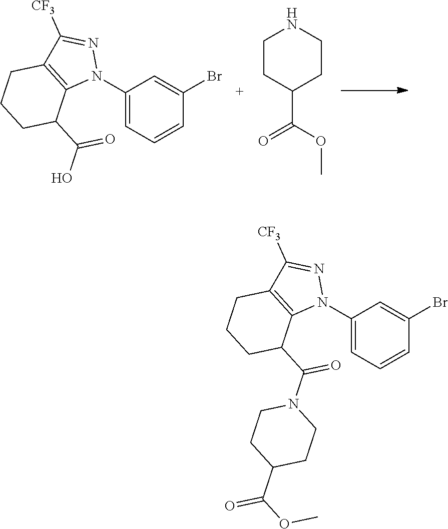

To a solution of 1-(3-bromophenyl)-3-(trifluoromethyl)-4,5,6,7-tetrahydroindazole-7-carboxylic acid (600 mg, 1.54 mmol) in DMF (10 mL) were added o-(7-azabenzotriazol-1-yl)-N,N,N′,N′-tetramethyluronium hexafluorophosphate (879 mg, 2.31 mmol), methyl isonipecotate (0.25 mL, 1.85 mmol), and triethylamine (0.64 mL, 4.63 mmol). The reaction was stirred at rt for 30 min. Then the mixture was diluted with water (20 mL) and extracted with EA (20 mL×3). The organic phase was washed with brine and dried over Na₂SO₄ and concentrated under vacuum. The residue was purified by flash column chromatography on silica gel (PE:EA=5:1) to give methyl 1-[1-(3-bromophenyl)-3-(trifluoromethyl)-4,5,6,7-tetrahydroindazole-7-carbonyl]piperidine-4-carboxylate (730 mg, 92.0%) as a white solid.

LCMS (Method 1): Retention time=2.10 min. MS (ESI) m/z 514.0, 516.0 [M+H]⁺.

### Step 7. Synthesis of methyl 1-[1-(3-hydroxyphenyl)-3-(trifluoromethyl)-4,5,6,7-tetrahydroindazole-7-carbonyl]piperidine-4-carboxylate

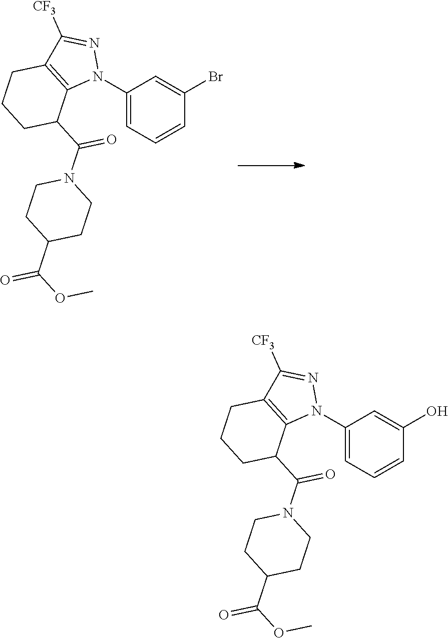 [SMILES: COC(=O)C1CCN(C(=O)C2CCCc3c(C(F)(F)F)nn(-c4cccc(Br)c4)c32)CC1 ; COC(=O)C1CCN(C(=O)C2CCCc3c(C(F)(F)F)nn(-c4cccc(O)c4)c32)CC1]

To a solution of methyl 1-[1-(3-bromophenyl)-3-(trifluoromethyl)-4,5,6,7-tetrahydroindazole-7-carbonyl]piperidine-4-carboxylate (250 mg, 0.486 mmol) in 1,4-dioxane (3 mL) were added water (0.044 mL, 2.43 mmol) and RockPhos Pd G3 (20 mg, 0.0243 mmol). The reaction mixture was stirred at 100° C. for 1.5 h under a nitrogen atmosphere. The mixture was concentrated and purified by reversed-phase flash chromatography (C18) (0.1% NH₄HCO₃ in water, 10-100% acetonitrile) to give methyl 1-[1-(3-hydroxyphenyl)-3-(trifluoromethyl)-4,5,6,7-tetrahydroindazole-7-carbonyl]piperidine-4-carboxylate (130 mg, 59.2% yield) as a white solid.

LCMS (Method 1): Retention time=1.90 min. MS (ESI) m/z 452.1 [M+H]⁺.

### Step 8. Synthesis of methyl 1-[1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylate

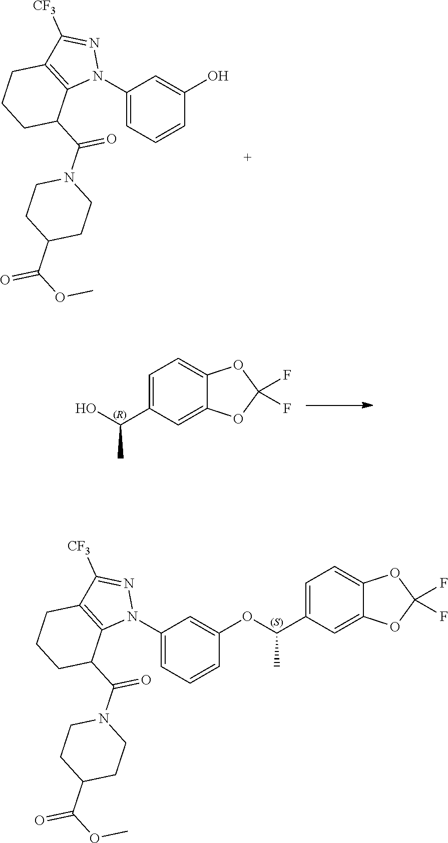

The title compound was obtained by following the procedures described in Step 1 of Example 104.

LCMS (Method 1): Retention time=2.26 min. MS (ESI) m/z 636.1 [M+H]⁺.

### Step 9. Synthesis of methyl 1-[(7S)-1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylate (P1) and methyl 1-[(7R)-1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylate (P2)

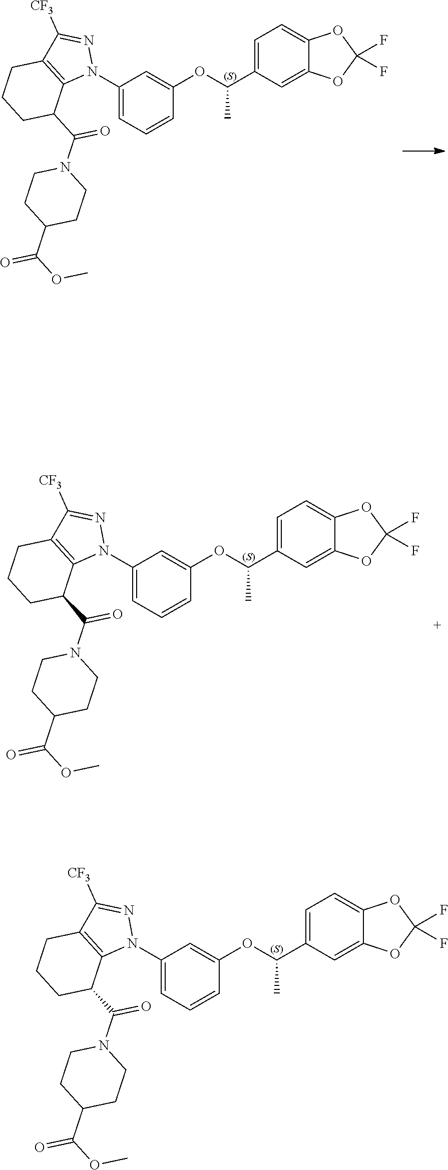

The crude of methyl 1-[1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylate (160 mg, 0.252 mmol) was separated by chiral-HPLC (IG 20*250 mm, 10 μm (Daicel)) to give methyl 1-[(7S)-1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylate (P1) (70 mg, 0.110 mmol, 43.7% yield) as a light yellow solid and methyl 1-[(7R)-1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylate (P2) (71 mg, 0.112 mmol, 44.3% yield) (P2) as a light yellow solid.

### P1:

LCMS (Method 1): Retention time=2.26 min. MS (ESI) m/z 636.1 [M+H]⁺.

Chiral Method: Column Name: IG 4.6*100 mm 5 μm Solvent: IPA [1% NH₃ (7M in MeOH)]; Find at 1.120 min; ee: 75.1%.

### P2:

LCMS (Method 1): Retention time=2.25 min. MS (ESI) m/z 636.1 [M+H]⁺.

Chiral Method: Column Name: IG 4.6*100 mm 5 μm Solvent: IPA [1% NH₃ (7M in MeOH)]; Find at 2.388 min; ee: 95.4%.

### Step 10a. Synthesis of 1-[(7S)-1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylic acid

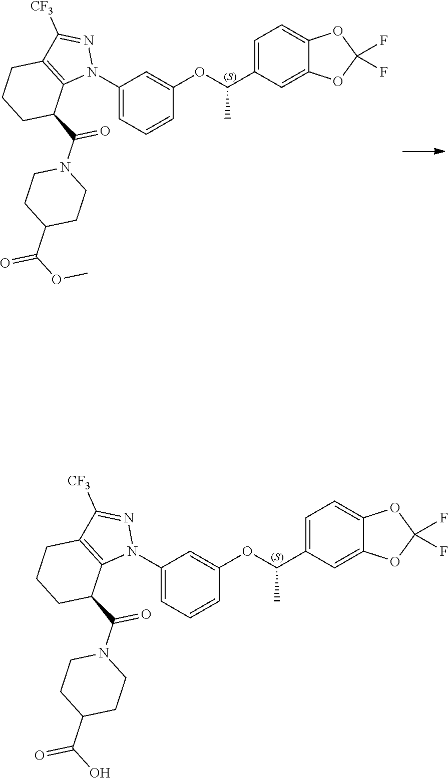

The title compound was obtained following the procedures described in Step 7a, Example 36.

LCMS (Method 4): Retention time=1.83 min. MS (ESI) m/z 622.1 [M+H]⁺.

Chiral Method: Column Name: IG 4.6*100 mm 5 μm Solvent: MeOH [0.2% NH₃ (7M in MeOH)]; Find at 0.937 min; ee: 80.82%.

¹H NMR (500 MHZ, MeOH-d₄) δ 7.35-7.12 (m, 4H), 6.96 (dq, J=14.1, 6.5 Hz, 3H), 5.56-5.45 (m, 1H), 4.37 (d, J=6.2 Hz, 1H), 4.15-3.73 (m, 2H), 3.08 (dd, J=24.7, 13.1 Hz, 1H), 2.76-2.32 (m, 4H), 2.15-2.05 (m, 1H), 2.00-0.90 (m, 10H).

### Step 10b. Synthesis of 1-[(7R)-1-[3-[(1S)-1-(2,2-difluoro-1,3-benzodioxol-5-yl)ethoxy]phenyl]-3-(trifluoromethyl)-4,5,6,7-tetrahydroindazole-7-carbonyl]piperidine-4-carboxylic acid

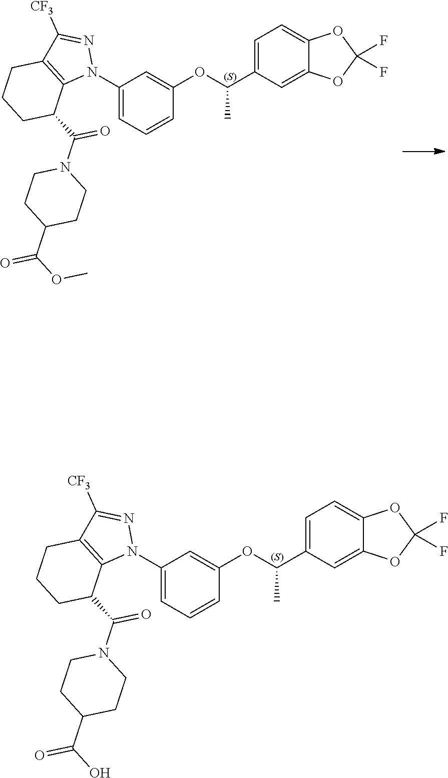

The title compound was obtained by following the same procedures as the above.

LCMS (Method 4): Retention time=1.82 min. MS (ESI) m/z 622.1 [M+H]⁺.

Chiral Method: Column Name: IG 4.6*100 mm 5 μm Solvent: MeOH [0.2% NH₃ (7M in MeOH)]; Find at 1.434 min; ee: 95.77%.

¹H NMR (500 MHZ, MeOH-d₄) δ 7.36-7.11 (m, 4H), 7.03-6.90 (m, 3H), 5.52 (p, J=6.2 Hz, 1H), 4.37 (dt, J=11.6, 6.1 Hz, 1H), 4.12-3.64 (m, 2H), 3.12-3.03 (m, 1H), 2.75-2.60 (m, 2H), 2.60-2.33 (m, 2H), 2.18-2.03 (m, 1H), 2.00-0.99 (m, 10H).

Example 121 (P1 and P2) to Example 123 were synthesized similarly to Example 120.

| Ex. | | | |
|---|---|---|---|
| No. | Structure | IUPAC name | LCMS/NMR |
| 121 (P1) | 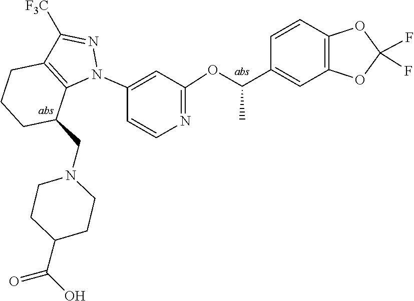 | 1-[(7S)-1-[2-[(1S)-1-(2,2- difluoro-1,3-benzodioxol-5- yl)ethoxy]-4-pyridyl]-3- (trifluoromethyl)-4,5,6,7- tetrahydroindazole-7- carbonyl]piperidine-4- carboxylic acid | LCMS (Method 2): Retention time = 1.73 min. MS (ESI) m/z 439.0 [M − 184]⁺. ¹H NMR (400 MHZ, MeOH-d₄) 8 8.22-8.10 (m, 1H), 7.35-7.28 (m, 1H), 7.23 (dd, J = 13.6, 5.2 Hz, 1H), 7.18-7.03 (m, 2H), 6.96-6.84 (m, 1H), 6.21 (dq, J = 27.2, 6.6 Hz, 1H), 4.65 (dd, J = 25.0, 6.6 Hz, 1H), 4.20-3.67 (m, 2H), 3.23 (d, J = 11.6 Hz, 1H), 2.81-2.44 (m, 4H), 2.25-2.11 (m, 1H), 2.01-1.57 (m, 9H), 1.30- 0.97 (m, 1H). |
| 121 (P2) | 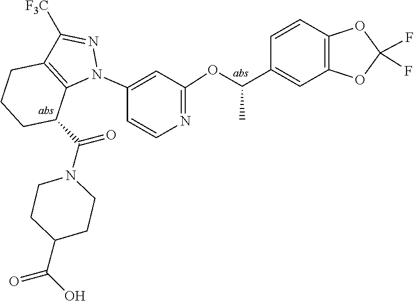 | 1-[(7R)-1-[2-[(1S)-1-(2,2- difluoro-1,3-benzodioxol-5- yl)ethoxy]-4-pyridyl]-3- (trifluoromethyl)-4,5,6,7- tetrahydroindazole-7- carbonyl]piperidine-4- carboxylic acid | LCMS (Method 2): Retention time = 1.73 min. MS (ESI) m/z 439.0 [M − 184]⁺. ¹H NMR (400 MHZ, MeOH-d₄) 8 8.17 (d, J = 5.6 Hz, 1H), 7.55- 7.01 (m, 4H), 6.87 (d, J = 1.5 Hz, 1H), 6.24 (dq, J = 41.2, 6.5 Hz, 1H), 4.60 (dt, J = 10.8, 6.1 Hz, 1H), 3.99 (m, 2H), 3.28- 3.15 (m, 1H), 2.84-2.07 (m, 5H), 2.00-1.55 (m, 9H), 1.40 (m, 1H). |
| 122 (P1) | 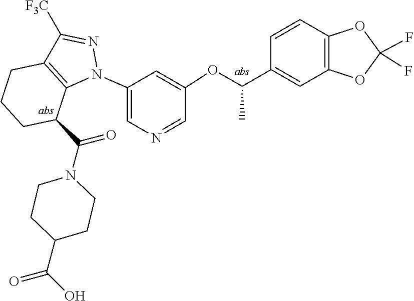 | 1-[(7S)-1-[5-[(1S)-1-(2,2- difluoro-1,3-benzodioxol-5- yl)ethoxy]-3-pyridyl]-3- (trifluoromethyl)-4,5,6,7- tetrahydroindazole-7- carbonyl]piperidine-4- carboxylic acid | LCMS (Method 4): Retention time = 1.54 min. MS (ESI) m/z 623.2 [M + H]⁺ ¹H NMR (400 MHZ, MeOH-d₄) 8 8.31 (d, J = 2.6 Hz, 1H), 8.20 (dd, J = 6.2, 1.8 Hz, 1H), 7.42 (dt, J = 24.3, 2.2 Hz, 1H), 7.32 (dd, J = 6.1, 1.2 Hz, 1H), 7.28- 7.22 (m, 1H), 7.17 (t, J = 8.0 Hz, 1H), 5.63 (p, J = 6.2 Hz, 1H), 4.48 (dt, J = 12.2, 6.4 Hz, 1H), 4.01-3.56 (m, 2H), 3.23-3.05 (m, 1H), 2.67 (t, J = 5.7 Hz, 2H), 2.55-2.38 (m, 2H), 2.23-2.05 (m, 1H), 2.00-1.61 (m, 8H), 1.48- 0.78 (m, 2H). |
| 122 (P2) | 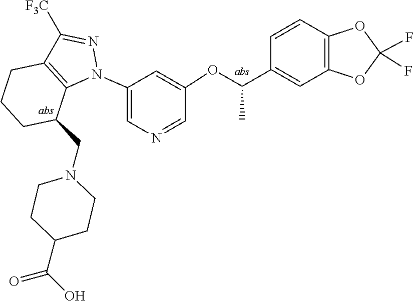 | 1-[(7R)-1-[5-[(1S)-1-(2,2- difluoro-1,3-benzodioxol-5- yl)ethoxy]-3-pyridyl]-3- (trifluoromethyl)-4,5,6,7- tetrahydroindazole-7- carbonyl]piperidine-4- carboxylic acid | LCMS (Method 4): Retention time = 1.54 min. MS (ESI) m/z 623.2 [M + H]⁺ ¹H NMR (400 MHz, MeOH-d₄) 8 8.29 (dd, J = 7.7, 2.6 Hz, 1H), 8.20 (dd, J = 8.3, 2.0 Hz, 1H), 7.47 (dd, J = 5.1, 2.8 Hz, 1H), 7.33 (dd, J = 5.4, 1.4 Hz, 1H), 7.29-7.15 (m, 2H), 5.68-5.56 (m, 1H), 4.47 (q, J = 6.0 Hz, 1H), 3.92 (ddd, J = 45.1, 34.9, 13.3 Hz, 2H), 3.21-3.09 (m, 1H), 2.68 (d, J = 5.6 Hz, 2H), 2.62- 2.38 (m, 2H), 2.23-2.10 (m, 1H), 1.99-1.62 (m, 8H), 1.52-0.90 (m, 2H). |
| 123 | 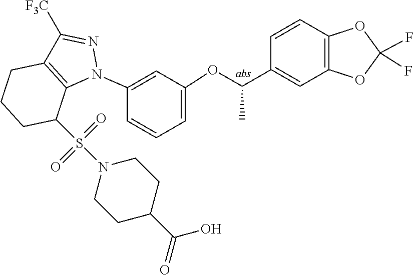 | 1-[1-[3-[(1S)-1-(2,2- difluoro-1,3-benzodioxol-5- yl)ethoxy]phenyl]-3- (trifluoromethyl)-4,5,6,7- tetrahydroindazol-7- yl]sulfonyl]piperidine-4- carboxylic acid | LCMS (Method 4): Retention time = 1.87 min. MS (ESI) m/z 658.1 [M + H]⁺. ¹H NMR (500 MHZ, MeOH-d₄) 8 7.40 (dt, J = 25.6, 8.1 Hz, 1H), 7.31 (d, J = 10.2 Hz, 1H), 7.29- 7.23 (m, 1H), 7.18 (dd, J = 27.8, 8.3 Hz, 1H), 7.10 (d, J = 7.4 Hz, 1H), 7.08-6.96 (m, 2H), 5.53 (dq, J = 24.1, 6.3 Hz, 1H), 4.86- 4.83 (m, 0.50H), 4.32 (d, J = 4.1 Hz, 0.50H), 3.28-2.87 (m, 2H), 2.83 (dd, J = 16.8, 6.0 Hz, 1H), 2.70-2.60 (m, 1H), 2.49-2.37 (m, 2H), 2.37-1.96 (m, 4H), 1.92- 1.84 (m, 1H), 1.72-1.58 (m, 5H), 1.55-1.42 (m, 1H), 1.41-1.29 (m, 1H). |

### Example 124 (P1 and P2)

### 1-[[(7R)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]methyl]piperidine-4-carboxylic acid

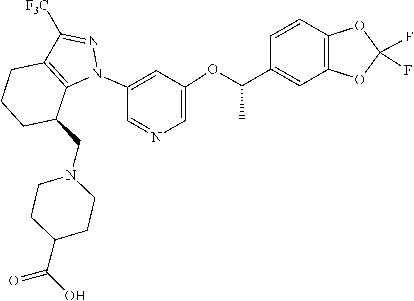 [SMILES: C[C@H](Oc1cncc(-n2nc(C(F)(F)F)c3c2[C@@H](CN2CCC(C(=O)O)CC2)CCC3)c1)c1ccc2c(c1)OC(F)(F)O2]

and

### 1-[[(7S)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]methyl]piperidine-4-carboxylic acid

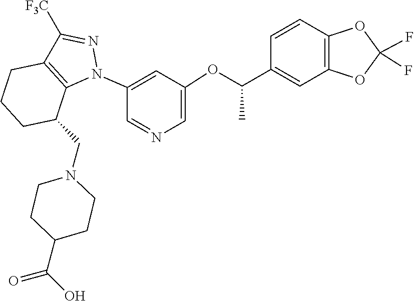 [SMILES: C[C@H](Oc1cncc(-n2nc(C(F)(F)F)c3c2[C@H](CN2CCC(C(=O)O)CC2)CCC3)c1)c1ccc2c(c1)OC(F)(F)O2]

### Step 1. Synthesis of methyl 1-(5-bromo-3-pyridyl)-3-(trifluoromethyl)-4,5,6,7-tetrahydroindazole-7-carboxylate

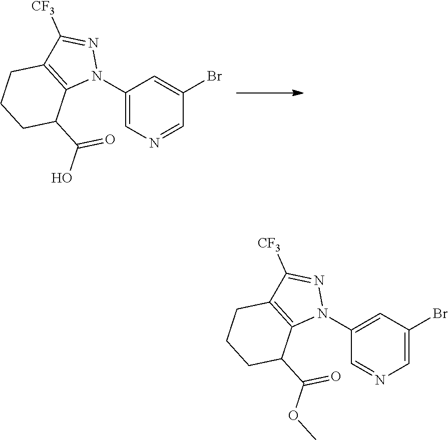 [SMILES: O=C(O)C1CCCc2c(C(F)(F)F)nn(-c3cncc(Br)c3)c21 ; COC(=O)C1CCCc2c(C(F)(F)F)nn(-c3cncc(Br)c3)c21]

To a solution of 1-(5-bromo-3-pyridyl)-3-(trifluoromethyl)-4,5,6,7-tetrahydroindazole-7-carboxylic acid (prepared similarly to Step 5 of Example 120) (2.00 g, 5.13 mmol) in methanol (60 mL) was added 3M HCl/MeOH (8.5 mL, 25.6 mmol) at rt. The reaction was stirred at 68° C. for 8 h. Then the mixture was concentrated under vacuum. The residue was purified by reversed-phase flash chromatography (C18) (0.1% NH₄HCO₃ in water, 10-100% acetonitrile) to give methyl 1-(5-bromo-3-pyridyl)-3-(trifluoromethyl)-4,5,6,7-tetrahydroindazole-7-carboxylate (2.00 g, 96.5%) as a white solid.

LCMS (Method 4): Retention time=2.01 min. MS (ESI) m/z 404.1, 406.1 [M+H]⁺.

### Step 2. Synthesis of [1-(5-bromo-3-pyridyl)-3-(trifluoromethyl)-4,5,6,7-tetrahydroindazol-7-yl]methanol

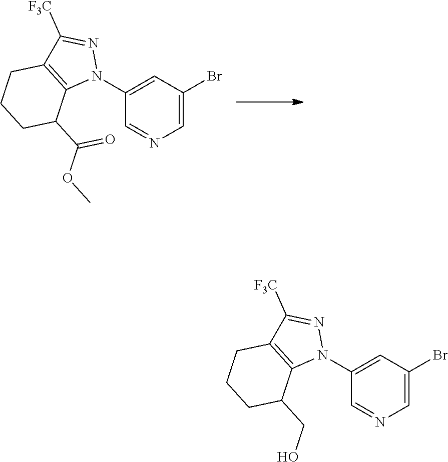 [SMILES: COC(=O)C1CCCc2c(C(F)(F)F)nn(-c3cncc(Br)c3)c21 ; OCC1CCCc2c(C(F)(F)F)nn(-c3cncc(Br)c3)c21]

To a solution of methyl 1-(5-bromo-3-pyridyl)-3-(trifluoromethyl)-4,5,6,7-tetrahydroindazole-7-carboxylate (2.00 g, 4.95 mmol) in THF (60 mL) was added lithium aluminum hydride (5.9 mL, 5.9 mmol) at 0° C. The reaction was stirred at 0° C. for 30 min. The mixture was then quenched by addition of water (0.23 mL), followed by 15% NaOH (0.46 mL) and water (0.69 mL). Na₂SO₄ was added and the mixture was stirred for a few minutes. The solid was removed by filtration through a Celite pad. The filtrate was concentrated under vacuum. The residue was purified by flash column chromatography on silica (eluting with PE:EA=1:1) to give [1-(5-bromo-3-pyridyl)-3-(trifluoromethyl)-4,5,6,7-tetrahydroindazol-7-yl]methanol (900 mg, 48.3%) as a colorless oil.

LCMS (Method 4): Retention time=1.82 min. MS (ESI) m/z 376.0, 378.0 [M+H]⁺.

### Step 3. Synthesis of [1-(5-bromo-3-pyridyl)-3-(trifluoromethyl)-4,5,6,7-tetrahydroindazol-7-yl]methyl methanesulfonate

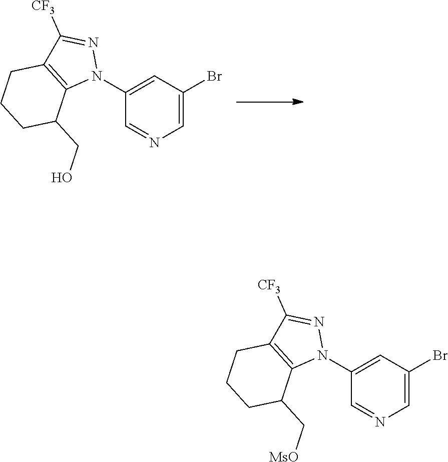 [SMILES: OCC1CCCc2c(C(F)(F)F)nn(-c3cncc(Br)c3)c21 ; CS(=O)(=O)OCC1CCCc2c(C(F)(F)F)nn(-c3cncc(Br)c3)c21]

To a solution of [1-(5-bromo-3-pyridyl)-3-(trifluoromethyl)-4,5,6,7-tetrahydroindazol-7-yl]methanol (900 mg, 2.39 mmol) and triethylamine (1.0 mL, 7.18 mmol) in DCM (20 mL) was added methanesulfonyl chloride (0.24 mL, 3.11 mmol) at 0° C. The reaction was stirred at rt for 4 h. Then the mixture was diluted with water (20 mL) and extracted with DCM (20 mL×2). The organic phase was washed with brine, dried over Na₂SO₄, and concentrated under vacuum. The residue was purified by flash column chromatography on silica (eluting with PE:EA=5:1) to give [1-(5-bromo-3-pyridyl)-3-(trifluoromethyl)-4,5,6,7-tetrahydroindazol-7-yl]methyl methanesulfonate (910 mg, 83.7%) as a colorless oil.

LCMS (Method 4): Retention time=1.89 min. MS (ESI) m/z 454.0, 456.0 [M+H]⁺.

### Step 4. Synthesis of methyl 1-[[1-(5-bromo-3-pyridyl)-3-(trifluoromethyl)-4,5,6,7-tetrahydroindazol-7-yl]methyl]piperidine-4-carboxylate

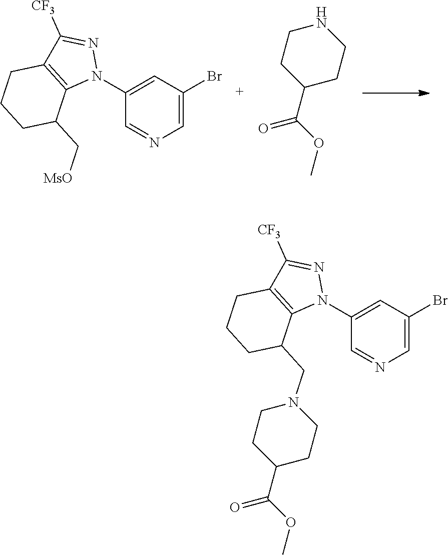

To a solution of [1-(5-bromo-3-pyridyl)-3-(trifluoromethyl)-4,5,6,7-tetrahydroindazol-7-yl]methyl methanesulfonate (910 mg, 2.00 mmol) and methyl isonipecotate (0.54 mL, 4.01 mmol) in acetonitrile (20 mL) was added potassium carbonate (1384 mg, 10.0 mmol) and potassium iodide (33 mg, 0.200 mmol). The reaction was stirred at 60° C. for 30 min. The reaction mixture was concentrated and purified by reverse-phase flash chromatography (C18) (0.1% NH₄HCO₃ in water, 10-100% acetonitrile) to give methyl 1-[[1-(5-bromo-3-pyridyl)-3-(trifluoromethyl)-4,5,6,7-tetrahydroindazol-7-yl]methyl]piperidine-4-carboxylate (500 mg, 49.7% yield) as a white solid.

LCMS (Method 4): Retention time=2.26 min. MS (ESI) m/z 501.1, 503.1 [M+H]⁺.

### Step 5. Synthesis of methyl 1-[[1-(5-hydroxy-3-pyridyl)-3-(trifluoromethyl)-4,5,6,7-tetrahydroindazol-7-yl]methyl]piperidine-4-carboxylate

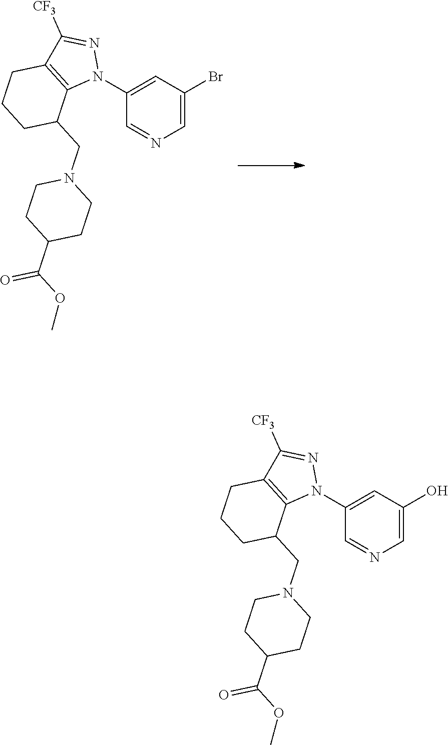 [SMILES: COC(=O)C1CCN(CC2CCCc3c(C(F)(F)F)nn(-c4cncc(Br)c4)c32)CC1 ; COC(=O)C1CCN(CC2CCCc3c(C(F)(F)F)nn(-c4cncc(O)c4)c32)CC1]

The title compound was obtained by following the protocols described in Step 7, Example 120.

LCMS (Method 4): Retention time=2.25 min. MS (ESI) m/z 501.1 [M+H]⁺.

### Step 6. Synthesis of methyl 1-[[1-(5-hydroxy-3-pyridyl)-3-(trifluoromethyl)-4,5,6,7-tetrahydroindazol-7-yl]methyl]piperidine-4-carboxylate

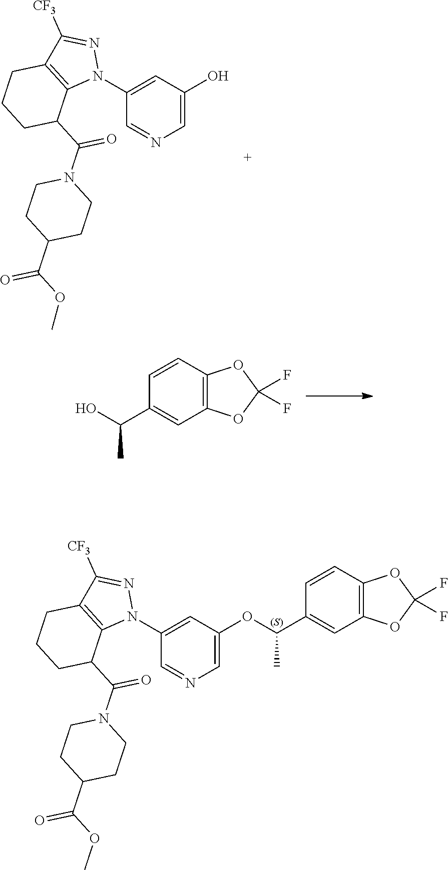

The title compound was obtained by following the protocols described in Step 8, Example 120.

LCMS (Method 4): Retention time=2.46 min. MS (ESI) m/z 623.3 [M+H]⁺.

### Step 7. Synthesis of methyl 1-[[(7R)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]methyl]piperidine-4-carboxylate (P1) and methyl 1-[[(7S)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]methyl]piperidine-4-carboxylate (P2)

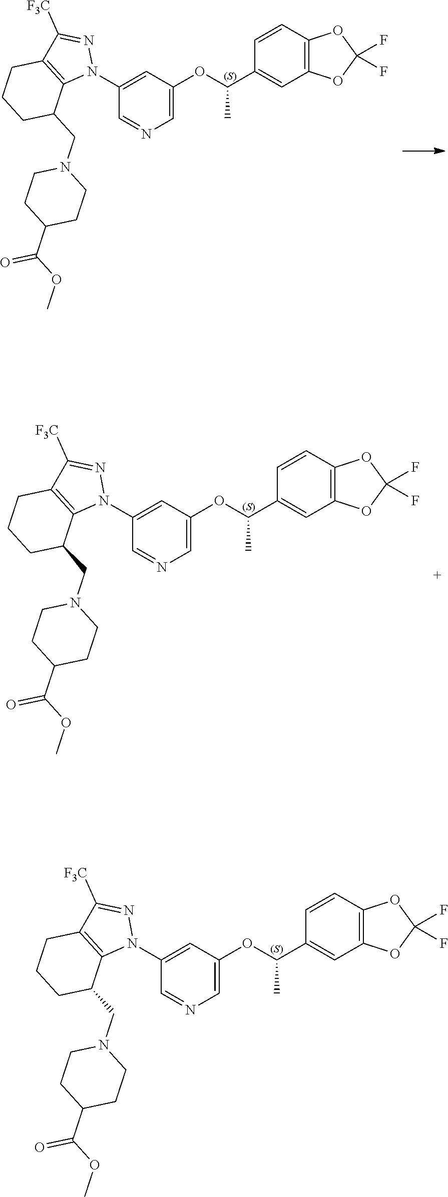

The crude of methyl 1-[1-[5-[[(1S)-1-(2,2-difluoro-1,3-benzodioxol-5-yl)ethoxy]-3-pyridyl]-3-(trifluoromethyl)-4,5,6,7-tetrahydroindazol-7-yl]methyl]piperidine-4-carboxylate (200 mg, 0.321 mmol) was separated by chiral-HPLC to give Fraction 1 (77 mg, 38.5% yield, ee: 100%) as a white solid and Fraction 2 (75 mg, 37.5%, ee: 99.47%) as a white solid.

### Fraction1:

LCMS (Method 4): Retention time=2.49 min. MS (ESI) m/z 623.3 [M+H]⁺.

Chiral Method: Column Name: (R,R) Whelk-O1 4.6*100 mm 3.5 μm Solvent: MeOH [0.1% NH₃ (7M in MeOH)]; Find at 2.060 min; ee: 100%.

### Fraction2:

LCMS (Method 4): Retention time=2.49 min. MS (ESI) m/z 623.3 [M+H]⁺.

Chiral Method: Column Name: (R,R) Whelk-O1 4.6*100 mm 3.5 μm Solvent: MeOH [0.1% NH₃ (7M in MeOH)]; Find at 2.060 min; ee: 99.47%.

### Step 8a. Synthesis of 1-[[(7R)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]methyl]piperidine-4-carboxylic acid (P1)

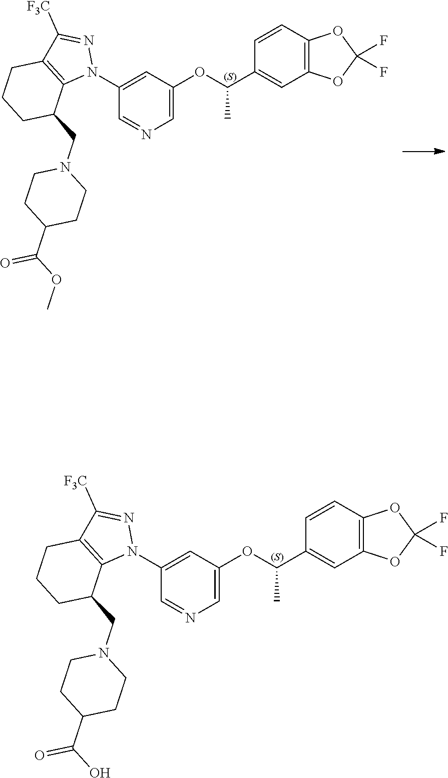

The hydrolysis was performed the same way as described in Step 10a of Example 120.

### P1:

LCMS (Method 4): Retention time=1.66 min. MS (ESI) m/z 609.2 [M+H]⁺.

Chiral Method: Column Name: (R,R) Whelk-O1 4.6*100 mm 3.5 μm; Solvent: EtOH [1% NH₃ (7M in MeOH)]; Find at 2.280 min; ee: 100%.

¹H NMR (400 MHZ, MeOH-d₄) δ 8.34 (dd, J=19.6, 2.2 Hz, 2H), 7.57 (t, J=2.2 Hz, 1H), 7.35 (d, J=1.4 Hz, 1H), 7.28 (dd, J=8.3, 1.5 Hz, 1H), 7.19 (d, J=8.3 Hz, 1H), 5.66 (q, J=6.3 Hz, 1H), 3.32 (s, 1H), 2.71 (d, J=16.5 Hz, 1H), 2.64-2.47 (m, 3H), 2.25 (dd, J=12.6, 9.6 Hz, 1H), 2.05 (dt, J=14.8, 7.5 Hz, 2H), 1.89-1.62 (m, 10H), 1.44 (dt, J=22.1, 6.1 Hz, 3H).

### Step 8b. Synthesis of 1-[[(7S)-1-[5-[(1S)-1-(2,2-difluoro-1,3-benzodioxol-5-yl)ethoxy]-3-pyridyl]-3-(trifluoromethyl)-4,5,6,7-tetrahydroindazol-7-yl]methyl]piperidine-4-carboxylic acid (P2)

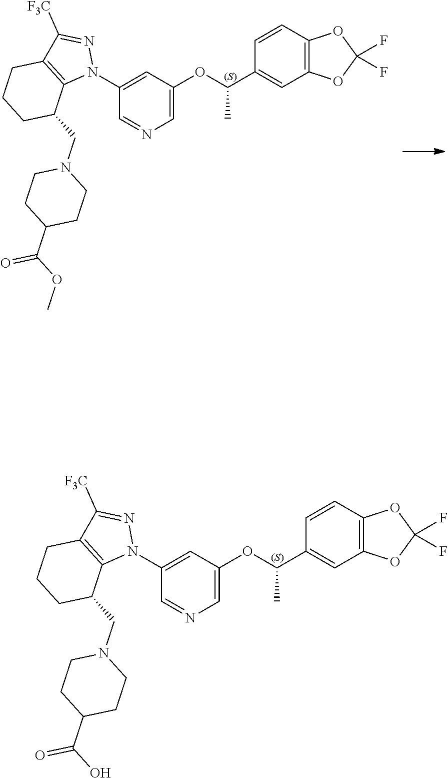

The hydrolysis was performed the same way as described in Step 10a of Example 120.

### P2:

LCMS (Method 4): Retention time=1.67 min. MS (ESI) m/z 609.2 [M+H]⁺.

Chiral Method: Column Name: (R,R) Whelk-O1 4.6*100 mm 3.5 μm Solvent: EtOH [1% NH₃ (7M in MeOH)]; Find at 2.968 min; ee: 100%.

¹H NMR (400 MHZ, MeOH-d₄) δ 8.34 (dd, J=24.1, 2.2 Hz, 2H), 7.53 (t, J=2.2 Hz, 1H), 7.39 (d, J=1.4 Hz, 1H), 7.31 (dd, J=8.3, 1.4 Hz, 1H), 7.22 (d, J=8.3 Hz, 1H), 5.60 (q, J=6.3 Hz, 1H), 3.00 (d, J=3.4 Hz, 1H), 2.79-2.66 (m, 1H), 2.62-2.50 (m, 1H), 2.47-2.35 (m, 1H), 2.33-2.16 (m, 2H), 2.07-1.91 (m, 3H), 1.88-1.55 (m, 9H), 1.52-1.36 (m, 2H), 1.22 (dd, J=22.0, 11.3 Hz, 1H).

### Example 125 (P1 and P2)

### 1-[[(7R)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]methyl]piperidine-4-carboxylic acid (P1)

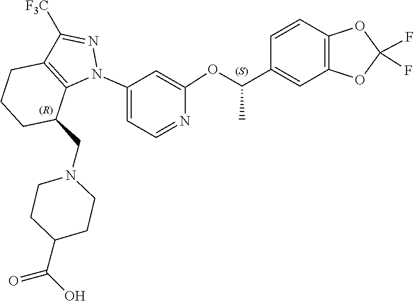

### 1-[[(7S)-1-[2-[(1S)-1-(2,2-difluoro-1,3-benzodioxol-5-yl)ethoxy]-4-pyridyl]-3-(trifluoromethyl)-4,5,6,7-tetrahydroindazol-7-yl]methyl]piperidine-4-carboxylic acid (P2)

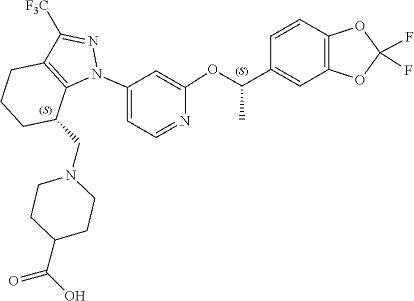

Example 125 (P1 and P2) were synthesized similarly as Example 124 (P1 and P2).

### P1:

LCMS (Method 2): Retention time=1.85 min. MS (ESI) m/z 609.2 [M+H]⁺.

Chiral Method: Column Name: IG 4.6*100 mm 5 μm Solvent: EtOH: n-Hexane=1:1 [0.2% NH₃ (7M in MeOH)]; Find at 2.649 min; ee: 97.88%

¹H NMR (400 MHZ, MeOH-d4) δ 8.21 (d, J=5.6 Hz, 1H), 7.33 (d, J=1.2 Hz, 1H), 7.27 (d, J=8.3 Hz, 1H), 7.21-7.13 (m, 2H), 7.11 (s, H), 6.26 (q, J=6.5 Hz, 1H), 3.56 (s, 1H), 2.86 (d, J=11.1 Hz, 1H), 2.78-2.50 (m, 3H), 2.45-2.32 (m, 1H), 2.08 (dd, J=12.8, 4.5 Hz, 3H), 2.03-1.51 (m, 11H), 1.39 (dd, J=21.3, 11.1 Hz, 1H).

### P2:

LCMS (Method 2): Retention time=1.85 min. MS (ESI) m/z 609.2 [M+H]⁺.

Chiral Method: Column Name: IG 4.6*100 mm 5 μm Solvent: EtOH: n-hexane=1:1 [0.2% NH₃ (7M in MeOH)]; Retention time=3.339 min; ee: 98.46%

¹H NMR (400 MHZ, MeOH-d₄) δ 8.22 (d, J=5.5 Hz, 1H), 7.34 (d, J=1.3 Hz, 1H), 7.27 (d, J=8.3 Hz, 1H), 7.17 (ddd, J=18.0, 7.7, 1.5 Hz, 3H), 6.23 (q, J=6.5 Hz, 1H), 3.52 (s, 1H), 2.85-2.52 (m, 4H), 2.45-2.35 (m, 1H), 2.16-2.00 (m, 4H), 1.93-1.69 (m, 6H), 1.67-1.45 (m, 5H).

### Example 126

### 4-[[(4S)-3-[6-[(1S)-1-(2,2-difluoro-1,3-benzodioxol-5-yl)ethoxy]-2-pyridyl]-1-(2,2,2-trifluoroethyl)-4,5,6,7-tetrahydroindazol-4-yl]oxy]benzoic acid

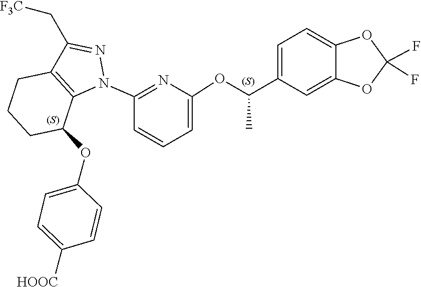

### Step 1. Synthesis of 3-iodo-1-(2,2,2-trifluoroethyl)-6,7-dihydro-5H-indazol-4-one

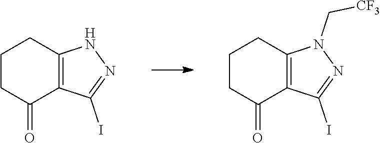 [SMILES: O=C1CCCc2[nH]nc(I)c21 ; O=C1CCCc2c1c(I)nn2CC(F)(F)F]

To a solution of 3-iodo-1,5,6,7-tetrahydroindazol-4-one (Intermediate D-11) (1000 mg, 3.82 mmol) in THF (20 mL) was added sodium (110 mg, 4.58 mmol). The reaction was stirred at 0° C. for 0.5 h. Then, 2,2,2-trifluoroethyl trifluoromethanesulfonate (1350 mg, 5.82 mmol) was added and the mixture was stirred at room temperature for 2 h. The reaction was quenched by the addition of water (50 mL). The solution was extracted with ethyl acetate (50 mL×3). The combined organic phases were combined, dried (Na₂SO₄), and concentrated to give a residue which was purified by flash column chromatography on silica gel (eluting with petroleum ether:ethyl acetate=5:1) to obtain 3-iodo-1-(2,2,2-trifluoroethyl)-6,7-dihydro-5H-indazol-4-one (600 mg, 43.4% yield) as an off-white solid.

LCMS (Method 2): Retention time=1.59 min. MS (ESI) m/z 345.1 [M+H]⁺.

### Step 5. Synthesis of 3-(6-fluoro-2-pyridyl)-1-(2,2,2-trifluoroethyl)-6,7-dihydro-5H-indazol-4-one

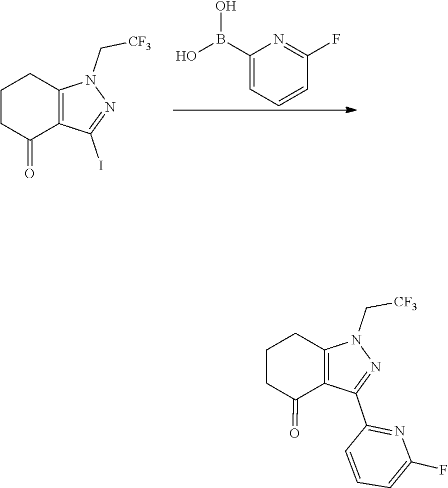 [SMILES: O=C1CCCc2c1c(I)nn2CC(F)(F)F ; OB(O)c1cccc(F)n1 ; O=C1CCCc2c1c(-c1cccc(F)n1)nn2CC(F)(F)F]

To a solution of 3-iodo-1-(2,2,2-trifluoroethyl)-6,7-dihydro-5H-indazol-4-one (100 mg, 0.29 mmol) in 1,4-dioxane (5 mL) and water (0.5 mL) was added (6-fluoro-2-pyridyl) boronic acid (100 mg, 0.71 mmol), cesium carbonate (300 mg, 0.92 mmol) and 1,1′-bis(diphenylphosphino)ferrocenedichloro palladium (II) dichloromethane complex (20 mg, 0.027 mmol). The reaction was stirred at 90° C. for 2 h. The mixture was concentrated to give a residue which was purified by flash column chromatography (petroleum ether: ethyl acetate=5:1) to obtain 3-(6-fluoro-2-pyridyl)-1-(2,2,2-trifluoroethyl)-6,7-dihydro-5H-indazol-4-one (50 mg, 52.1% yield) as a light yellow solid.

LCMS (Method 1): Retention time=1.80 min. MS (ESI) m/z 314.1 [M+H]⁺.

### Step 6. Synthesis of 3-(6-hydroxy-2-pyridyl)-1-(2,2,2-trifluoroethyl)-6,7-dihydro-5H-indazol-4-one

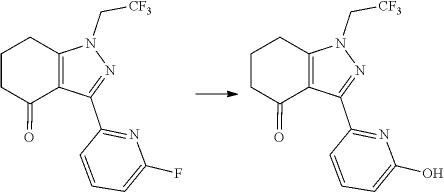 [SMILES: O=C1CCCc2c1c(-c1cccc(F)n1)nn2CC(F)(F)F ; O=C1CCCc2c1c(-c1cccc(O)n1)nn2CC(F)(F)F]

To a solution of 3-(6-fluoro-2-pyridyl)-1-(2,2,2-trifluoroethyl)-6,7-dihydro-5H-indazol-4-one (100 mg, 0.32 mmol) in 1,4-dioxane (1 mL) was added aqueous HCl (12 N, 0.5 mL) and stirred at 90° C. for 2 h. The mixture was concentrated to give a residue which was purified by flash column chromatography (dichloromethane:methanol=10:1) to obtain 3-(6-hydroxy-2-pyridyl)-1-(2,2,2-trifluoroethyl)-6,7-dihydro-5H-indazol-4-one (75 mg, 71.7% yield) as a light-yellow solid.

LCMS (Method 2): Retention time=1.47 min. MS (ESI) m/z 312.3 [M+H]⁺.

### Step 7. Synthesis of 3-[6-[(1S)-1-(2,2-difluoro-1,3-benzodioxol-5-yl)ethoxy]-2-pyridyl]-1-(2,2,2-trifluoroethyl)-6,7-dihydro-5H-indazol-4-one

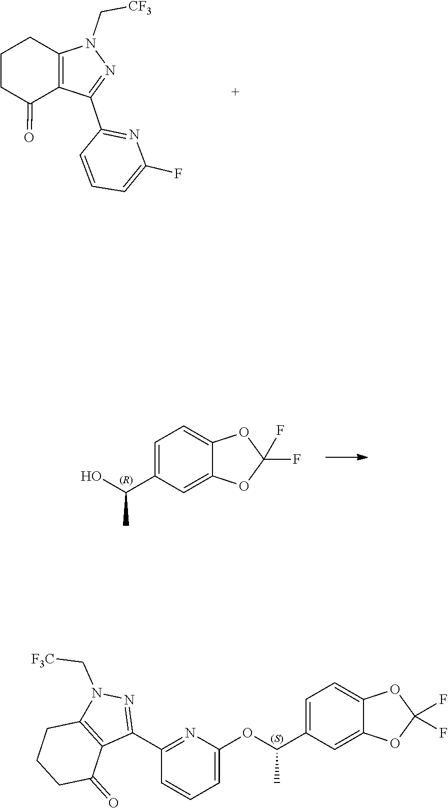

To a solution of 3-(6-hydroxy-2-pyridyl)-1-(2,2,2-trifluoroethyl)-6,7-dihydro-5H-indazol-4-one (200 mg, 0.64 mmol) in THF (5 mL) was added (1R)-1-(2,2-difluoro-1,3-benzodioxol-5-yl)ethanol (Intermediate C-1R) (156 mg, 0.77 mmol) and triphenylphosphine (337 mg, 1.29 mmol). Then the mixture was cooled to 0° C. under N₂, and diethyl azodicarboxylate (0.15 mL, 0.96 mmol) was added drop wise at 0° C. The mixture was stirred at rt for 1 h. The mixture was concentrated and the crude was then purified by flash column chromatography (EtOAc in PE: 0-100% for 50 min) to obtain 3-[6-[(1S)-1-(2,2-difluoro-1,3-benzodioxol-5-yl)ethoxy]-2-pyridyl]-1-(2,2,2-trifluoroethyl)-6,7-dihydro-5H-indazol-4-one (220 mg, 69.1% yield) as a white solid.

LCMS (Method 2): Retention time=2.35 min. MS (ESI) m/z 496.2 [M+H]⁺.

### Step 8. Synthesis of (4R)-3-[6-[(1S)-1-(2,2-difluoro-1,3-benzodioxol-5-yl)ethoxy]-2-pyridyl]-1-(2,2,2-trifluoroethyl)-4,5,6,7-tetrahydroindazol-4-ol

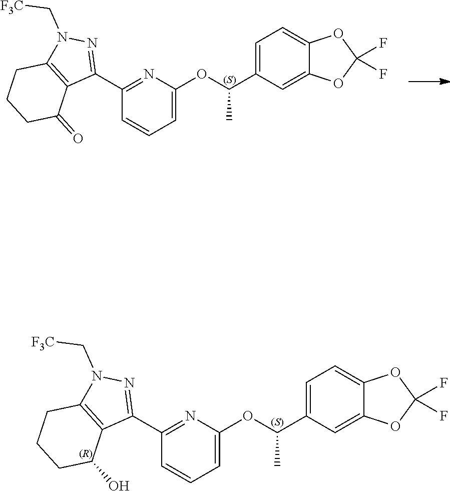

The enantio-selective reduction reaction was carried out following the same procedures as described in Step 1, Example 109.

LCMS (Method 2): Retention time=2.72 min. MS (ESI) m/z 498.2 [M+H]⁺.

### Step 9. Synthesis of (4R)-3-[6-[(1S)-1-(2,2-difluoro-1,3-benzodioxol-5-yl)ethoxy]-2-pyridyl]-1-(2,2,2-trifluoroethyl)-4,5,6,7-tetrahydroindazol-4-ol

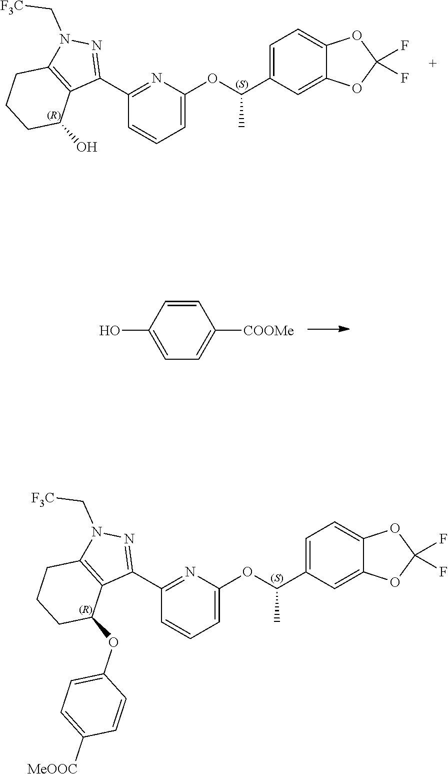

The title compound was obtained by following the protocols described in Step 2, Example 36.

LCMS (Method 2): Retention time=1.99 min. MS (ESI) m/z 632.0 [M+H]⁺.

### Step 10. Synthesis of 4-[[(4S)-3-[6-[(1S)-1-(2,2-difluoro-1,3-benzodioxol-5-yl)ethoxy]-2-pyridyl]-1-(2,2,2-trifluoroethyl)-4,5,6,7-tetrahydroindazol-4-yl]oxy]benzoic acid

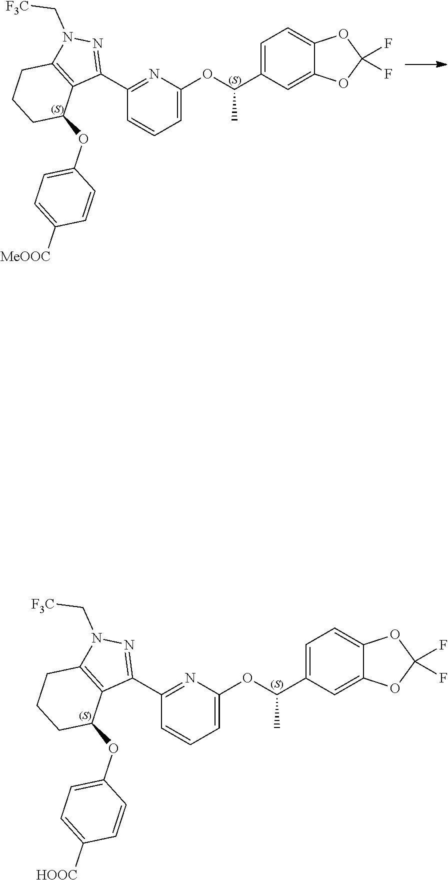

The ester hydrolysis was performed by following the protocol described in Step 4, Example 36.

LCMS (Method 2): Retention time=1.80 min. MS (ESI) m/z 616.2 [M+H]⁺.

¹H NMR (400 MHZ, MeOH-d₄) δ:7.91 (d, J=8.6 Hz, 2H), 7.67-7.53 (m, 1H), 7.47 (d, J=7.5 Hz, 1H), 7.02 (s, 1H), 6.95 (dd, J=8.4, 5.4 Hz, 3H), 6.74 (d, J=8.2 Hz, 1H), 6.64 (d, J=8.2 Hz, 1H), 6.03 (s, 1H), 5.94 (q, J=6.5 Hz, 1H), 5.02-4.89 (m, 2H), 2.93 (dd, J=16.7, 4.4 Hz, 1H), 2.79-2.53 (m, 1H), 2.43 (d, J=14.0 Hz, 1H), 1.98 (d, J=47.8 Hz, 2H), 1.73 (t, J=14.1 Hz, 1H), 1.40 (d, J=6.4 Hz, 3H).

### Example 127

### 4-[[(4S)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-yl]oxy]benzoic acid

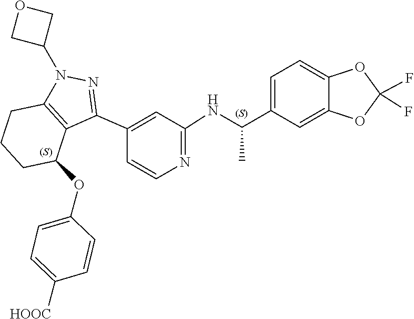

### Step 1. Synthesis of 3-iodo-1-(oxetan-3-yl)-6,7-dihydro-5H-indazol-4-one

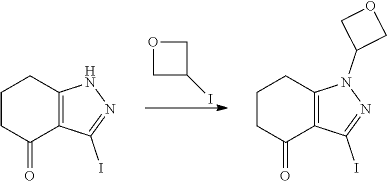 [SMILES: O=C1CCCc2[nH]nc(I)c21 ; IC1COC1 ; O=C1CCCc2c1c(I)nn2C1COC1]

To a solution of 3-iodo-1,5,6,7-tetrahydroindazol-4-one (Intermediate D-11) (1000 mg, 3.82 mmol) in DMF (20 mL) was added 3-iodooxetane (2100 mg, 11.40 mmol) and cesium carbonate (3700 mg, 11.40 mmol). The reaction was stirred at 90° C. for 2 h. The mixture was concentrated to give a residue which was purified by flash column chromatography (petroleum ether:ethyl acetate=5:1) to obtain 3-iodo-1-(oxetan-3-yl)-6,7-dihydro-5H-indazol-4-one (300 mg, 23.5%) as a light-yellow solid.

LCMS (Method 1): Retention time=1.53 min. MS (ESI) m/z 319.0 [M+H]⁺.

### Step 2. Synthesis of 4-bromo-N-[(1S)-1-(2,2-difluoro-1,3-benzodioxol-5-yl)ethyl]pyridine-2-amine

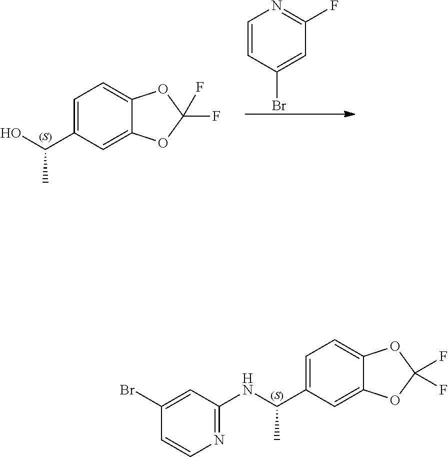

To a solution of(S)-1-(2,2-difluorobenzo[d][1,3]dioxol-5-yl)ethan-1-amine (Intermediate C-6a) (2000 mg, 9.94 mmol) in DMSO (100 mL) was added 4-bromo-2-fluoro-pyridine (5300 mg, 30.10 mmol) and cesium fluoride (4500 mg, 29.60 mmol). The reaction was stirred at 100° C. for 12 h. The mixture was diluted with water (500 mL) and extracted with ethyl acetate (100 mL×3). The combined organic phase was concentrated to give a residue which was purified by flash column chromatography (petroleum ether:ethyl acetate=5:1) to obtain 4-bromo-N-[(1S)-1-(2,2-difluoro-1,3-benzodioxol-5-yl)ethyl]pyridine-2-amine (2000 mg, 53.5%) as a colorless oil.

LCMS (Method 2): Retention time=1.96 min. MS (ESI) m/z 357.1, 359.1 [M+H]⁺.

### Step 3. Synthesis of N-[(1S)-1-(2,2-difluoro-1,3-benzodioxol-5-yl)ethyl]-4-(4,4,5,5-tetramethyl-1,3,2-dioxaborolan-2-yl)pyridine-2-amine

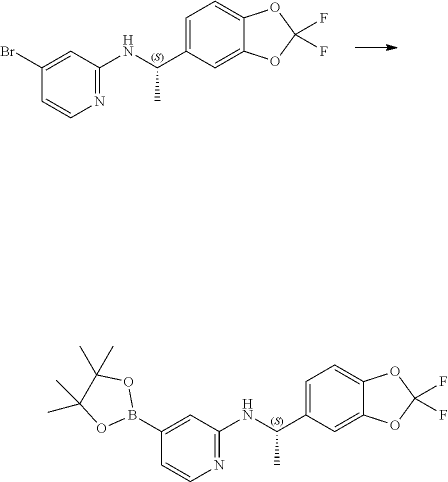

To a solution of 4-bromo-N-[(1S)-1-(2,2-difluoro-1,3-benzodioxol-5-yl)ethyl]pyridine-2-amine (2000 mg, 5.60 mmol) in 1,4-dioxane (40 mL) was added bis(pinacolato)diboron (2150 mg, 8.47 mmol) and potassium acetate (1650 mg, 16.80 mmol) and 1,1′-bis(diphenylphosphino)ferrocenedichloro palladium (II) dichloromethane complex (400 mg, 0.54 mmol) under N₂. The reaction was stirred at 90° C. for 2 h. The mixture was filtered and the filtrate was concentrated to obtain N-[(1S)-1-(2,2-difluoro-1,3-benzodioxol-5-yl)ethyl]-4-(4,4,5,5-tetramethyl-1,3,2-dioxaborolan-2-yl)pyridine-2-amine (1200 mg, 50.36%) which was used directly in the next reaction step without further purification.

LCMS (Method 2): Retention time=1.62 min. MS (ESI) m/z 323.2 [M+H]⁺.

### Step 4. Synthesis of 3-[2-[[(1S)-1-(2,2-difluoro-1,3-benzodioxol-5-yl)ethyl]amino]-4-pyridyl]-1-(oxetan-3-yl)-6,7-dihydro-5H-indazol-4-one

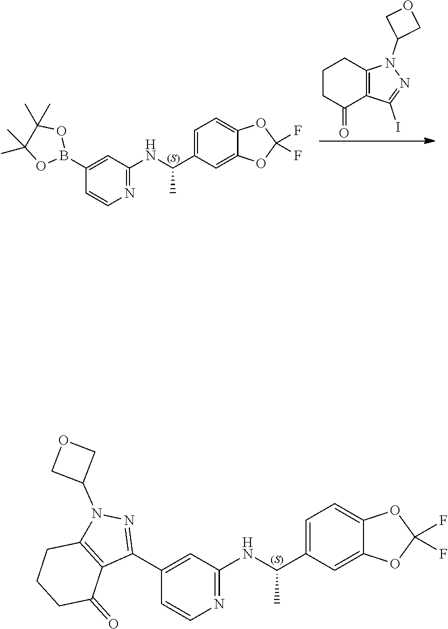

To a solution of N-[(1S)-1-(2,2-difluoro-1,3-benzodioxol-5-yl)ethyl]-4-(4,4,5,5-tetramethyl-1,3,2-dioxaborolan-2-yl)pyridine-2-amine (200 mg, 0.63 mmol) in 1,4-dioxane (5 mL) and water (0.5 mL) were added cesium carbonate (500 mg, 1.53 mmol), 3-iodo-1-(oxetan-3-yl)-6,7-dihydro-5H-indazol-4-one (200 mg, 0.49 mmol) and 1,1′-bis(diphenylphosphino)ferrocenedichloro palladium (II) dichloromethane complex (36 mg, 0.049 mmol) under N₂. The reaction was stirred at 90° C. for 1 h. The mixture was concentrated to give a residue which was purified by flash column chromatography on silica (eluting with petroleum ether/ethyl acetate=5/1) to obtain 3-[2-[[(1S)-1-(2,2-difluoro-1,3-benzodioxol-5-yl)ethyl]amino]-4-pyridyl]-1-(oxetan-3-yl)-6,7-dihydro-5H-indazol-4-one (150 mg, 61.48%) as a light yellow solid.

LCMS (Method 1): Retention time=1.47 min. MS (ESI) m/z 469.2 [M+H]⁺.

### Step 5. Synthesis of (4R)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-ol

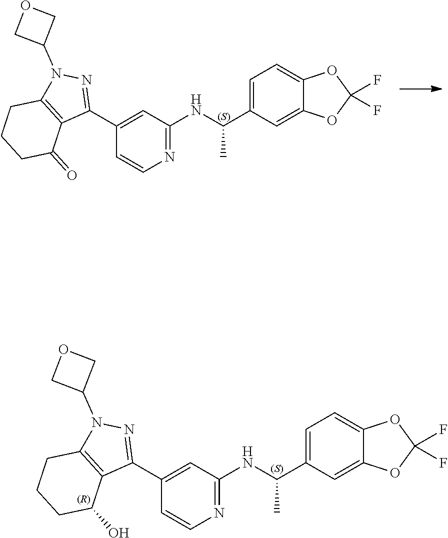

To a solution of 3-[2-[[(1S)-1-(2,2-difluoro-1,3-benzodioxol-5-yl)ethyl]amino]-4-pyridyl]-1-(oxetan-3-yl)-6,7-dihydro-5H-indazol-4-one (200 mg, 0.42 mmol) in 2-propanol (1.0 mL, 13.00 mmol) was added [N-[(1R,2R)-2-(amino-κN)-1,2-diphenylethyl]-4-methylbenzenesulfonamidato-κN]chloro[(1,2,3,4,5,6-n)-1-methyl-4-(1-methylethyl)benzene]-Ruthenium (CAS #192139-92-7) (50 mg), followed by triethylamine (0.60 mL, 4.30 mmol) and formic acid (0.40 mL, 10.40 mmol) under N₂ at room temperature. The reaction was stirred at 50° C. for 2 h. The mixture was concentrated to give a residue which was purified by flash column chromatography (dichloromethane:methanol=10:1) to obtain (4R)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-ol (100 mg, 47.30%) as a white solid.

LCMS (Method 1): Retention time=1.51 min. MS (ESI) m/z 471.2 [M+H]⁺.

### Step 6. Synthesis of methyl 4-[[(4S)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-yl]oxy]benzoate

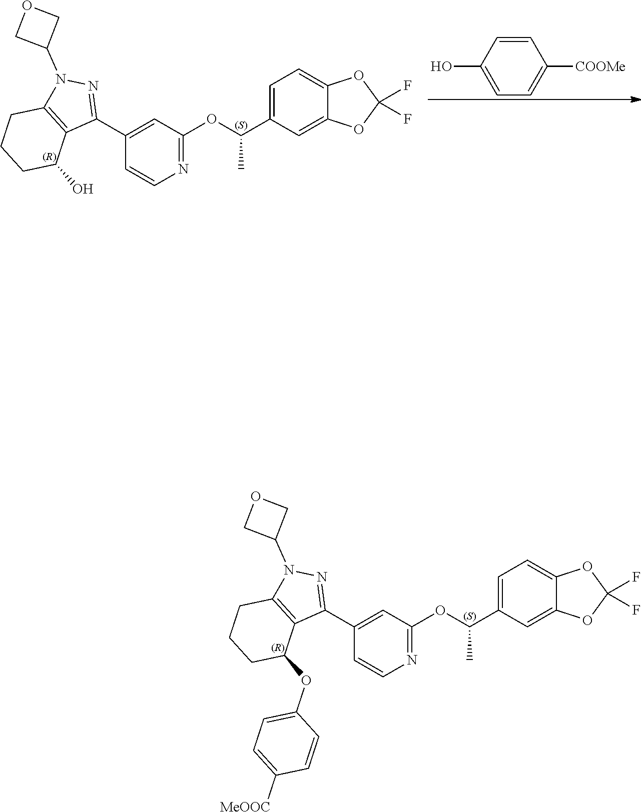

To a solution of (4R)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-ol (100 mg, 0.21 mmol) in THF (5 mL) was added methyl 4-hydroxybenzoate (49 mg, 0.32 mmol) and triphenylphosphine (170 mg, 0.64 mmol), followed by slow addition of diethyl azodicarboxylate (0.051 mL, 0.32 mmol) at 0° C. The reaction was stirred at room temperature for 2 h. The mixture was concentrated to give a residue which was purified by flash column chromatography (petroleum ether/ethyl acetate=1/1) to obtain methyl 4-[[(4S)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-yl]oxy]benzoate (30 mg, 22.2) as a light yellow solid.

LCMS (Method 2): Retention time=2.08 min. MS (ESI) m/z 605.0 [M+H]⁺.

### Step 7. Synthesis of 4-[[(4S)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-yl]oxy]benzoic acid

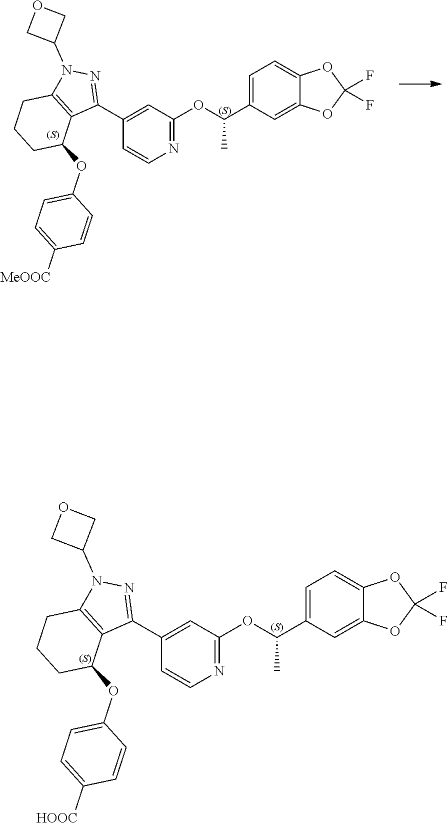

To the solution of methyl 4-[[(4S)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-yl]oxy]benzoate (30 mg, 0.049 mmol) in methanol (0.5 mL), water (0.5 mL), and THF (1 mL) was added lithium hydroxide (4.0 mg, 0.17 mmol). The reaction was stirred at room temperature for 6 h. To the mixture was added citric acid aqueous solution and extracted with ethyl acetate (10 mL×3). The combined organic phase was concentrated to give a residue which was purified by Prep-HPLC to give 4-[[(4S)-3-[2-[[(1S)-1-(2,2-difluoro-1,3-benzodioxol-5-yl)ethyl]amino]-4-pyridyl]-1-(oxetan-3-yl)-4,5,6,7-tetrahydroindazol-4-yl]oxy]benzoic acid (7.0 mg, 23.6%) as a white solid.

LCMS (Method 2): Retention time=1.49 min. MS (ESI) m/z 591.3 [M+H]⁺.

¹H NMR (400 MHZ, DMSO-d₆) δ:12.93-12.25 (m, 1H), 7.92 (d, J=8.7 Hz, 2H), 7.78 (d, J=5.3 Hz, 1H), 7.26 (d, J=8.1 Hz, 2H), 7.15 (d, J=8.8 Hz, 2H), 7.07 (d, J=8.4 Hz, 1H), 6.98 (d, J=8.2 Hz, 1H), 6.89 (s, 1H), 6.66 (d, J=5.1 Hz, 1H), 5.74 (s, 1H), 5.57 (dd, J=14.0, 6.8 Hz, 1H), 5.04-4.97 (m, 2H), 4.92 (t, J=7.1 Hz, 2H), 4.78 (s, 1H), 2.83 (d, J=16.5 Hz, 1H), 2.55 (d, J=8.5 Hz, 1H), 2.11 (d, J=12.1 Hz, 1H), 1.81 (s, 2H), 1.66 (s, 1H), 1.31 (d, J=6.9 Hz, 3H).

Example 128 and Example 129 were synthesized similarly to Example 127.

### Example 128

### 4-[[(4S)-3-[2-[[(1S)-1-(2,2-difluoro-1,3-benzodioxol-5-yl)ethyl]amino]-4-pyridyl]-1-isopropyl-4,5,6,7-tetrahydroindazol-4-yl]oxy]benzoic acid

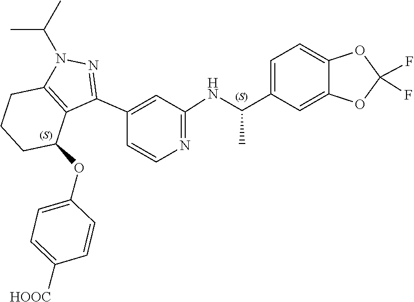

LCMS (Method 2): Retention time=1.60 min. MS (ESI) m/z 577.0 [M+H]⁺.

¹H NMR (400 MHZ, DMSO-d₆) δ:7.92 (d, J=8.7 Hz, 2H), 7.74 (d, J=5.1 Hz, 1H), 7.25 (d, J=8.3 Hz, 2H), 7.15 (d, J=8.7 Hz, 2H), 7.05 (d, J=9.6 Hz, 1H), 6.90 (d, J=8.0 Hz, 1H), 6.79 (s, 1H), 6.60 (d, J=6.4 Hz, 1H), 5.72 (s, 1H), 4.73 (s, 1H), 4.49 (d, J=6.7 Hz, 1H), 2.87 (d, J=16.4 Hz, 1H), 2.54 (m, 1H), 2.10 (m, 1H), 1.83 (m, 2H), 1.73-1.62 (m, 1H), 1.43 (dd, J=12.9, 6.6 Hz, 6H), 1.29 (d, J=6.8 Hz, 3H).

### Example 129

### 4-[[(4S)-3-[2-[[(1S)-1-(2,2-difluoro-1,3-benzodioxol-5-yl)ethyl]amino]-4-pyridyl]-1-(2,2,2-trifluoroethyl)-4,5,6,7-tetrahydroindazol-4-yl]oxy]benzoic acid

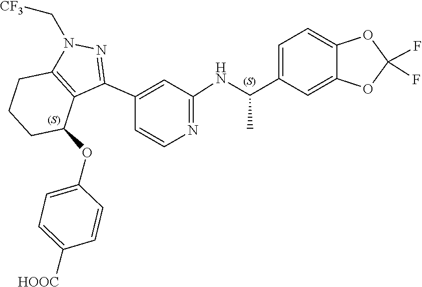

LCMS (Method 2): Retention time=1.68 min. MS (ESI) m/z 617.0 [M+H]⁺.

¹H NMR (400 MHZ, DMSO-d₆) δ:7.92 (d, J=8.7 Hz, 2H), 7.78 (d, J=5.3 Hz, 1H), 7.25 (d, J=8.2 Hz, 2H), 7.15 (d, J=8.8 Hz, 2H), 7.04 (d, J=8.4 Hz, 1H), 6.95 (d, J=8.1 Hz, 1H), 6.80 (s, 1H), 6.63 (d, J=5.3 Hz, 1H), 5.75 (s, 1H), 5.20-5.07 (m, 2H), 4.76 (s, 1H), 2.92 (d, J=16.5 Hz, 1H), 2.57 (dd, J=14.7, 7.5 Hz, 1H), 2.10 (t, J=11.5 Hz, 1H), 1.84 (s, 2H), 1.72 (d, J=10.5 Hz, 1H), 1.30 (d, J=6.8 Hz, 3H).

### ΔF508-CFTR-HRP Trafficking Assay

Test agents were screened for their effect on cell surface expression of ΔF508-CFTR using the ΔF508-CFTR-HRP trafficking assay. For this assay, a ΔF508-CFTR expression construct was designed with an in-frame insertion of a horseradish peroxidase (HRP) tag into the 4th extracellular loop of CFTR. The HRP tag would be presented extracellularly, in cells transiently transfected with the ΔF508-CFTR-HRP construct, following correction of the ΔF508-CFTR trafficking defect. The quantity of ΔF508-CFTR-HRP expressed on the plasma membrane could therefore be determined by measuring an HRP-dependent signal after the application of a cell-impermeable HRP substrate.

In brief, CF submucosal gland epithelial cells (CFSMEo-), derived from the airways of a CF patient (ΔF508/Q2X) and provided by Dr. Dieter Gruenert, University of California-San Francisco, were cultured at 37° C. with 5% CO₂ in minimum essential medium (MEM) with Earle's salt and nonessential amino acids, supplemented with 10% (volume per volume [v/v]) fetal bovine serum, 2 mM L-glutamine and 1× (v/v) penicillin/streptomycin and grown in tissue culture-treated flasks coated with an extracellular matrix (ECM) cocktail consisting of 10 μg/mL human fibronectin, 30 μg/mL bovine collagen type I, and 100 μg/mL bovine serum albumin in LHC basal medium. These CFSMEo⁻ cells were transiently transfected with 4 μg of the ΔF508-CFTR-HRP expression construct using the Lonza 4D-Nucleofector Core unit (Lonza, Catalogue No. AAF-1002B) with the X unit (Lonza, Catalogue No. AAF-1002X) and SF Cell Line 4D-Nucleofector X Kit L (Lonza, Catalogue No. V4XC-2024) according to the manufacturer's instructions and plated at 1.2×10⁴ cells/well onto collagen-coated, 96-well plates (Corning Inc.). Twenty-four h post-transfection, cells were treated with test agents, solubilized in DMSO at a concentration of 3 μM in replicates of 3, and allowed to incubate for 48 h. To detect HRP expression, cell culture media was removed, cells were washed with PBS and 100 μL of Luminata Forte HRP chemiluminescent substrate (EMD Millipore, Burlington, MA) was added to each well. Following a 5-minute incubation, chemiluminescence was measured in relative luminescent units (RLUs) using an EnVision plate reader (Perkin Elmer, Waltham, MA).

The ΔF508-CFTR-HRP trafficking data was normalized to the vehicle treated control (0.3% DMSO), followed by calculation of the mean, standard deviation, and standard error. If the test agent increased cell surface expression of ΔF508-CFTR-HRP relative to the vehicle-treated control, then that test agent was considered a ΔF508-CFTR corrector. Based on the description above, the biological activity of the examples in this disclosure is collected in Table 2.

TABLE 2
ΔF508-CFTR-HRP trafficking data normalized to
the vehicle treated control (0.3% DMSO)*

| Compound Number | RLUs* |
|---|---|
| 1 | C |
| 2 | C |
| 3 | C |
| 4 | B |
| 5 | A |
| 6 | C |
| 7 | A |
| 8 | B |
| 9 | A |
| 10 | B |
| 11 | A |
| 12 | C |
| 13 | B |
| 14 | A |
| 15 | B |
| 16 | C |
| 17 | C |
| 18 | A |
| 19 | B |
| 20 | B |
| 21 | A |
| 22 | B |
| 23 | C |
| 24 | B |
| 25 | B |
| 26 | A |
| 27 | C |
| 28 | A |
| 29 | B |
| 30 | B |
| 31 | C |
| 32 | B |
| 33 | B |
| 34 | B |
| 35 | B |
| 36 | C |
| 36a | A |
| 36b | A |
| 36c | B |
| 36d | B |
| 36e | A |
| 36f | A |
| 36g | B |
| 36h | B |
| 36i | A |
| 36j | C |
| 36k | C |
| 36l | A |
| 36m | A |
| 37 | A |
| 38 | A |
| 39 | A |
| 40 | A |
| 41 | A |
| 42 | A |
| 43 | A |
| 44 | A |
| 45 | C |
| 46 | B |
| 47 | B |
| 48 | C |
| 49 | B |
| 50 | A |
| 51 | A |
| 52 | C |
| 53 | A |
| 54 | C |
| 55 | C |
| 56 | B |
| 57 | B |
| 58 | B |
| 59 | C |
| 60 | C |
| 61 | C |
| 62 | C |
| 63 | B |
| 64 | A |
| 65 | C |
| 66 | A |
| 67 | A |
| 68 | C |
| 69 | B |
| 70 | A |
| 71 | C |
| 72 | B |
| 73 | A |
| 74 (P1) | A |
| 74 (P2) | A |
| 75 (P1) | C |
| 75 (P2) | C |
| 76 (P1) | C |
| 76 (P2) | C |
| 77 (P1) | B |
| 77 (P2) | B |
| 78 (P1) | C |
| 78 P2) | C |
| 79 (P1) | B |
| 79 (P2) | A |
| 80 (P1) | A |
| 80 (P2) | B |
| 81 (P1) | A |
| 81 (P2) | C |
| 82 (P1) | B |
| 82 (P2) | A |
| 83 (P1) | B |
| 83 (P2) | C |
| 84 (P1) | B |
| 84 (P2) | C |
| 85 (P1) | C |
| 85 (P2) | B |
| 86 (P1) | C |
| 86 (P2) | C |
| 87 | A |
| 88 | C |
| 89 | C |
| 90 | C |
| 91 | A |
| 92 | B |
| 93 | A |
| 94 (P1) | C |
| 94 (P2) | C |
| 95 (P1) | C |
| 95 (P2) | C |
| 96 (P1) | B |
| 96 (P2) | C |
| 97 (P1) | C |
| 97 (P2) | C |
| 98 (P1) | C |
| 98 (P2) | C |
| 99 (P1) | C |
| 99 (P2) | C |
| 100 (P1) | A |
| 100 (P2) | C |
| 101 (P1) | C |
| 101 (P2) | C |
| 102 (P1) | C |
| 102 (P2) | B |
| 103 (P1) | B |
| 103 (P2) | C |
| 104 | B |
| 105 (P1) | B |
| 105 (P2) | B |
| 106 (P1) | B |
| 106 (P2) | C |
| 107 (P1) | C |
| 107 (P2) | C |
| 108 (P1) | C |
| 108 (P2) | C |
| 109 | A |
| 110 | B |
| 111 | C |
| 112 | A |
| 113 | C |
| 114 | C |
| 115 (P1) | B |
| 115 (P2) | C |
| 116 | B |
| 117 | C |
| 118 | C |
| 119 | C |
| 120 (P1) | B |
| 120 (P2) | C |
| 121 (P1) | A |
| 121 (P2) | C |
| 122 (P1) | C |
| 122 (P2) | B |
| 123 | B |
| 124 (P1) | B |
| 124 (P2) | B |
| 125 (P1) | A |
| 125 (P2) | A |
| 126 | A |
| 127 | A |
| 128 | B |
| 129 | B |

*A: RLU 5.0 μM-10 μM; B: RLU 3.5 μM-5.0 μM; C: RLU 0.0 μM-3.5 μM
Ref.
1. Joncour, Agnes; Decor, Anne; Liu, Jian-Miao; Dau, Marie-Elise Tran Huu; Baudoin, Olivier, Chemistry-A European Journal, 13(19), 5450-5465, S5450/1-S5450/56 (2007).
2. Takao Ikariya, Shohei Hashiguchi, Kunihiko Murata, and Ryoji Noyori., Organic Syntheses, Vol. 82, p10-17 (2005); Coll. Vol. 11, p17-24 (2009).

What is claimed is:

1. A compound represented by Formula (I) or Formula (II): 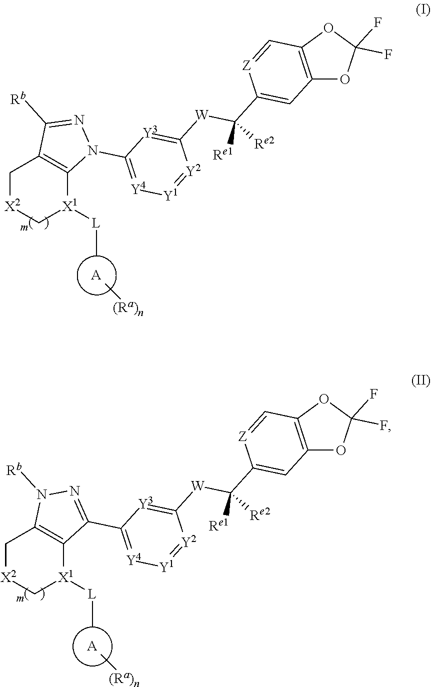
or a pharmaceutically acceptable salt thereof
A is selected from the group consisting of optionally substituted 3-10 membered carbocyclyl, optionally substituted 3-10 membered heterocyclyl, optionally substituted phenyl, and optionally substituted 5-10 membered heteroaryl;
L is selected from the group consisting of a bond, —O—, —S—, —S(O)₂—, —CH₂—, —C(O)—, —N(H)—, and —N(H)C(O)—;
W is selected from the group consisting of —O—, —S—, —S(O)—, —S(O)₂—, —N(H)—, and optionally substituted 3-6 membered heterocyclylene;
X¹ is N or C(H);
X² is selected from the group consisting of —CH₂—, —N(H)—, and —N(C₁₋₃ alkyl)-;
Y¹ is selected from the group consisting of —C(H)═, —C(F)═, and —N═;
Y² is selected from the group consisting of —C(H)═, —C(F)═, and —N═;
Y³ is selected from the group consisting of —C(H)═, —C(F)═, and —N═;
Y⁴ is selected from the group consisting of —C(H)═, —C(F)═, and —N═;
Z is selected from the group consisting of —C(H)═, —C(F)═, and —N═;
each R^{a} is independently selected from the group consisting of halogen, optionally substituted C₁-C₃ aliphatic, optionally substituted 5-7 membered heterocyclyl, optionally substituted 5-6 membered heteroaryl, and —COOH;
R^{b} is selected from the group consisting of —CN, optionally substituted C₁-C₆ aliphatic, optionally substituted C₁-C₆ haloaliphatic, and optionally substituted 3-7 membered heterocyclyl;
R^{e1} is selected from the group consisting of hydrogen, C₁-C₅-alkyl, and C₃-C₅-cycloalkyl;
R^{e2} is selected from the group consisting of hydrogen, C₁-C₅-alkyl, and C₃-C₈-cycloalkyl;
wherein R^{e1} and R^{e2}, together with the atom on which they are attached, are optionally taken together to form an optionally substituted 3-6 membered carbocyclyl ring;
m is 0-2; and
n is 0-5.

2. The compound of claim 1, wherein the compound is represented by (I-a) or (II-a) 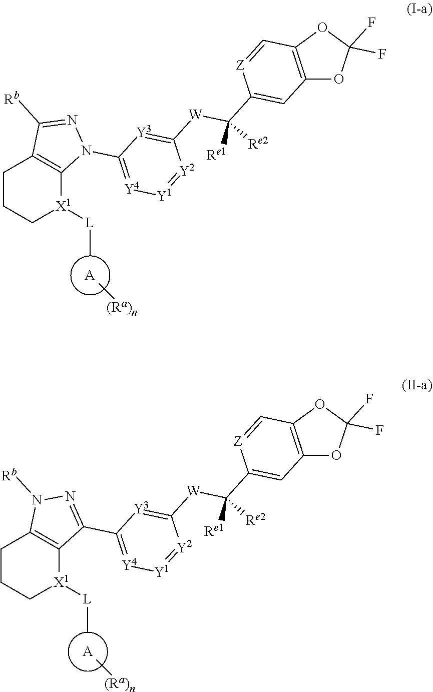
or a pharmaceutically acceptable salt thereof.

3. The compound of claim 1 or 2, wherein W is —O—.

4. The compound of claim 1 or 2, wherein W is —S—.

5. The compound of claim 1 or 2, wherein W is —S(O)—.

6. The compound of claim 1 or 2, wherein W is —S(O)₂—.

7. The compound of claim 1 or 2, wherein W is —N(H)—.

8. The compound of claim 1 or 2, wherein W is optionally substituted 3-6 membered heterocyclylene.

9. The compound of claim 8, wherein W is optionally substituted 3-4 membered heterocyclylene.

10. The compound of claim 9, wherein W is

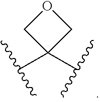 [SMILES: *C1(*)COC1]

11. The compound of any of claims 1-10, wherein
Y¹ is —N═;
Y² is —C(H)═ or —C(F)═;
Y³ is —C(H)═; and
Y⁴ is —C(H)═.

12. The compound of any of claims 1-10, wherein
Y¹ is —N═;
Y² is —N═;
Y³ is —C(H)═; and
Y⁴ is —C(H)═.

13. The compound of any of claims 1-10, wherein
Y¹ is —C(H)═;
Y² is —N═;
Y³ is —C(H)═; and
Y⁴ is —C(H)═.

14. The compound of any of claims 1-10, wherein
Y¹ is —C(H)═;
Y² is —C(H)═ or —C(F)═;
Y³ is —C(H)═; and
Y⁴ is —C(H)═ or —C(F)═.

15. The compound of any of claims 1-10, wherein
Y¹ is —C(H)═;
Y² is —C(H)═ or —C(F)═;
Y³ is —N═; and
Y⁴ is —C(H)═ or —C(F)═.

16. The compound of any of claims 1-15, wherein the compound is represented by Formula (I-a1) or (I-a2) 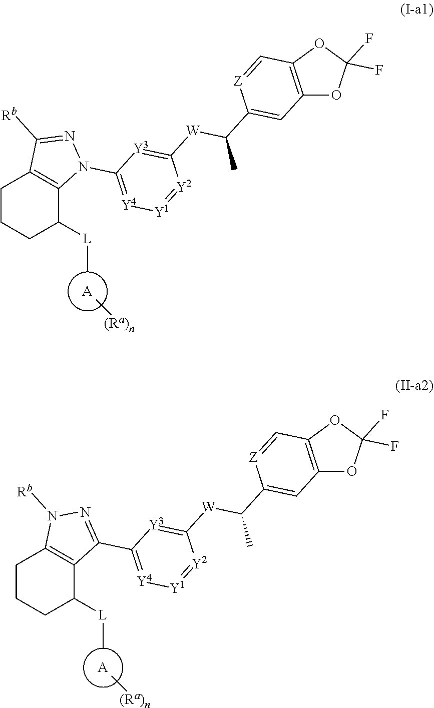
or a pharmaceutically acceptable salt thereof.

17. The compound of claim 2 or 16, wherein Y¹ is —C(H)═ or —N═, and Y² is —C(H)═ or —N═.

18. The compound of claim 2 or 16, wherein Y¹ is —C(H)═ and Y² is —C(H)═.

19. The compound of claim 2 or 16, wherein Y¹ is —N═ and Y² is —C(H)═.

20. The compound of claim 2 or 16, wherein Y¹ is —C(H)═ and Y² is —N═.

21. The compound of claim 2 or 16, wherein Y¹ is —N═ and Y² is —N═.

22. The compound of any of claims 1-15, wherein X¹ is C.

23. The compound of any of claims 1-15, wherein X¹ is N.

24. The compound of any of claims 1-23, wherein L is —O—.

25. The compound of any of claims 1-23, wherein L is —N(H)—.

26. The compound of any of claims 1-23, wherein L is —N(H)C(O)—.

27. The compound of any of claims 1-23, wherein L is selected from the group consisting of —S(O)₂—, —CH₂—, and —C(O)—.

28. The compound of any of claims 1-23, wherein L is —CH₂—.

29. The compound of any of claims 1-28, wherein A is selected from the group consisting of optionally substituted 5-8 membered carbocyclyl, optionally substituted 5-7 membered heterocyclyl, and optionally substituted phenyl.

30. The compound of any of claims 1-28, wherein A is selected from the group consisting of optionally substituted cyclohexyl, optionally substituted bicyclo[1.1.1]pentyl, optionally substituted bicyclo[2.2.2]octyl, optionally substituted piperidinyl, and optionally substituted phenyl.

31. The compound of any of claims 1-28, wherein A is selected from the group consisting of

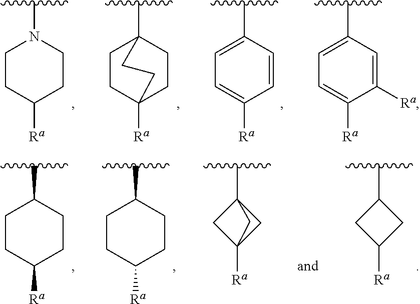

32. The compound of any of claims 1-31, wherein each R^{a} is independently selected from the group consisting of methyl and —COOH.

33. The compound of any of claims 1-32, wherein n is 1-5 and at least one instance of R^{a} is —COOH.

34. The compound of claim 31, wherein R^{a} is —COOH.

35. The compound of any of claims 1-31, wherein n is 1 and R^{a} is optionally substituted 5-6 membered heterocyclyl.

36. The compound of claim 31 or 35, wherein R^{a} is

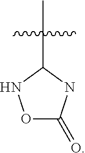

37. The compound of any of claims 1-36, wherein R^{b} is —CN.

38. The compound of any of claims 1-36, wherein R^{b} is C₁-C₃ haloaliphatic.

39. The compound of claim 38, wherein R^{b} is —CF₃.

40. The compound of claim 38, wherein R^{b} is —CH₂CF₃.

41. The compound of any of claims 1-36, wherein R^{b} is optionally substituted C₁-C₃ aliphatic.

42. The compound of claim 41, wherein R^{b} is iso-propyl.

43. The compound of any of claims 1-36, wherein R^{b} is optionally substituted 3-5 heterocyclyl.

44. The compound of claim 43, wherein R^{b} is optionally substituted oxetanyl.

45. The compound of any of claims 1-44, wherein Z is —C(H)═.

46. The compound of any of claims 1-44, wherein Z is —N═.

47. The compound of any of claims 1-44, wherein Z is —C(F)═.

48. A compound selected from the group consisting of

| Ex. No. | Structure | IUPAC name |
|---|---|---|
| 1 | 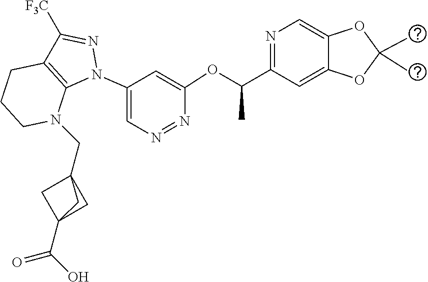 [SMILES: C[C@@H](Oc1cc(-n2nc(C(F)(F)F)c3c2N(CC24CC(C(=O)O)(C2)C4)CCC3)cnn1)c1cc2c(cn1)OC(C)(C)O2] | 3-[[1-[6-[(1R)-1-(2,2-difluoro- [1,3]dioxolo[4,5-c]pyridin-6- yl)ethoxy]pyridazin-4-yl]-3- (trifluoromethyl)-5,6-dihydro-4H- pyrazolo[3,4-b]pyridin-7- yl]methyl]bicyclo[1.1.1]pentane-1- carboxylic acid |
| 2 | 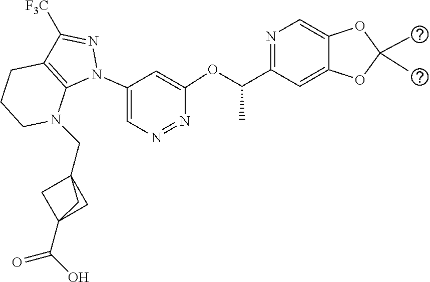 [SMILES: C[C@H](Oc1cc(-n2nc(C(F)(F)F)c3c2N(CC24CC(C(=O)O)(C2)C4)CCC3)cnn1)c1cc2c(cn1)OC(C)(C)O2] | 3-[[1-[6-[(1S)-1-(2,2-difluoro- [1,3]dioxolo[4,5-c]pyridin-6- yl)ethoxy]pyridazin-4-yl]-3- (trifluoromethyl)-5,6-dihydro-4H- pyrazolo[3,4-b]pyridin-7- yl]methyl]bicyclo[1.1.1]pentane-1- carboxylic acid |
| 3 | 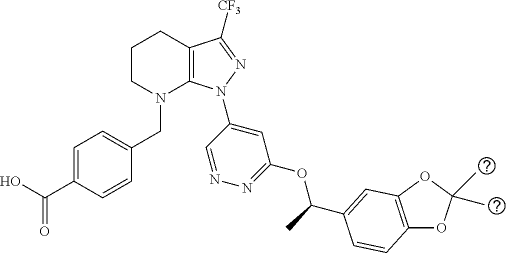 [SMILES: C[C@@H](Oc1cc(-n2nc(C(F)(F)F)c3c2N(Cc2ccc(C(=O)O)cc2)CCC3)cnn1)c1ccc2c(c1)OC(C)(C)O2] | 4-[[1-[6-[(1R)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]benzoic acid |
| 4 | 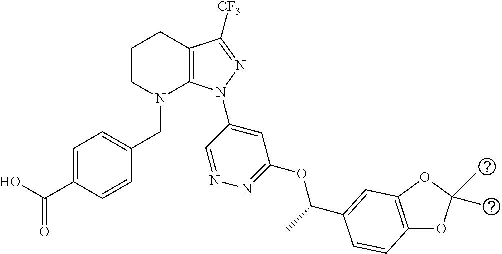 [SMILES: C[C@H](Oc1cc(-n2nc(C(F)(F)F)c3c2N(Cc2ccc(C(=O)O)cc2)CCC3)cnn1)c1ccc2c(c1)OC(C)(C)O2] | 4-[[1-[6-[(1S)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]benzoic acid |
| 5 | 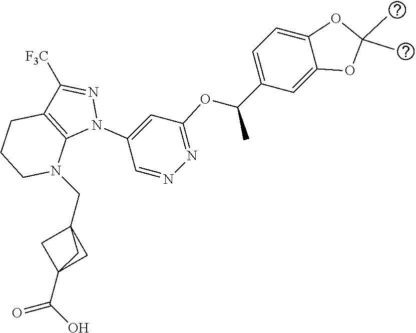 [SMILES: C[C@@H](Oc1cc(-n2nc(C(F)(F)F)c3c2N(CC24CC(C(=O)O)(C2)C4)CCC3)cnn1)c1ccc2c(c1)OC(C)(C)O2] | 3-[[1-[6-[(1R)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]bicyclo[1.1.1]pentane-1- carboxylic acid |
| 6 | 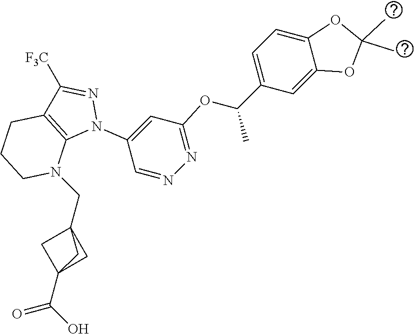 [SMILES: C[C@H](Oc1cc(-n2nc(C(F)(F)F)c3c2N(CC24CC(C(=O)O)(C2)C4)CCC3)cnn1)c1ccc2c(c1)OC(C)(C)O2] | 3-[[1-[6-[(1S)-1-(2,2-difluoro-1,3- benzodioxol-5-yl)ethoxy]pyridazin-4- yl]-3-(trifluoromethyl)-5,6-dihydro-4H- pyrazolo[3,4-b]pyridin-7- yl]methyl]bicyclo[1.1.1]pentane-1- carboxylic acid |
| 7 | 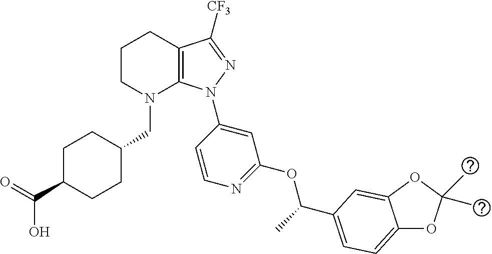 [SMILES: C[C@H](Oc1cc(-n2nc(C(F)(F)F)c3c2N(C[C@H]2CC[C@H](C(=O)O)CC2)CCC3)ccn1)c1ccc2c(c1)OC(C)(C)O2] | Trans-4-[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]cyclohexanecarboxylic acid |
| 8 | 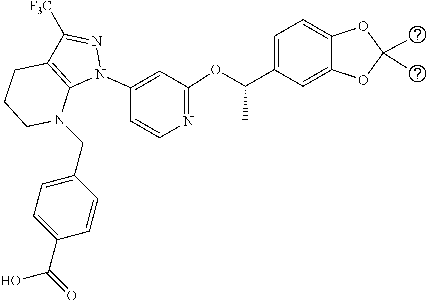 [SMILES: C[C@H](Oc1cc(-n2nc(C(F)(F)F)c3c2N(Cc2ccc(C(=O)O)cc2)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]benzoic acid |
| 9 | 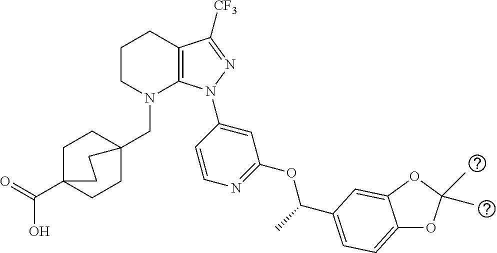 [SMILES: C[C@H](Oc1cc(-n2nc(C(F)(F)F)c3c2N(CC24CCC(C(=O)O)(CC2)CC4)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-5,6-dihydro-4H- pyrazolo[3,4-b]pyridine-7- yl]methyl]bicyclo[2.2.2]octane-1- carboxylic acid |
| 10 | 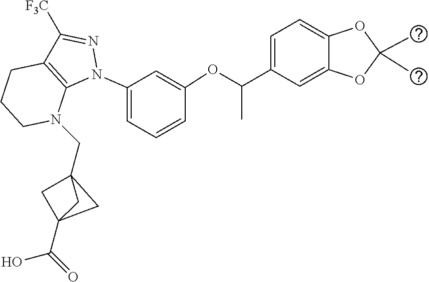 [SMILES: CC(Oc1cccc(-n2nc(C(F)(F)F)c3c2N(CC24CC(C(=O)O)(C2)C4)CCC3)c1)c1ccc2c(c1)OC(C)(C)O2] | 3-[[1-[3-[1-(2,2-difluoro-1,3- benzodioxol-5-yl)ethoxy]phenyl]-3- (trifluoromethyl)-5,6-dihydro-4H- pyrazolo[3,4-b]pyridine-7- yl]methyl]bicyclo[1.1.1]pentane-1- carboxylic acid |
| 11 | 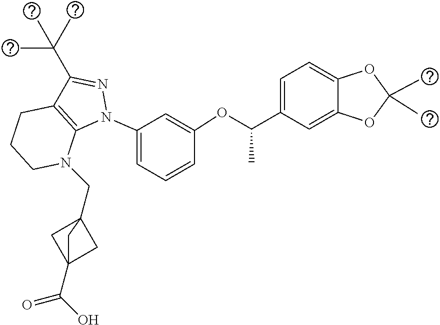 [SMILES: C[C@H](Oc1cccc(-n2nc(C(C)(C)C)c3c2N(CC24CC(C(=O)O)(C2)C4)CCC3)c1)c1ccc2c(c1)OC(C)(C)O2] | 3-[[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- yl]methyl]bicyclo[1.1.1]pentane-1- carboxylic acid |
| 12 | 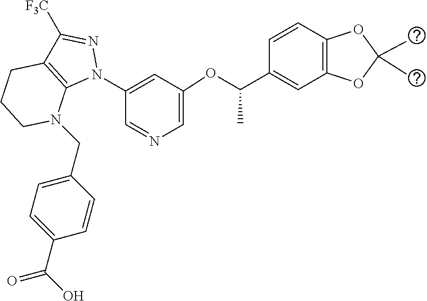 [SMILES: C[C@H](Oc1cncc(-n2nc(C(F)(F)F)c3c2N(Cc2ccc(C(=O)O)cc2)CCC3)c1)c1ccc2c(c1)OC(C)(C)O2] | 4-[[1-[5-[(1S)-1-(2,2-difluoro-1,3- benzodioxol-5-yl)ethoxy]-3-pyridyl]-3- (trifluoromethyl)- 5,6-dihydro-4H-pyrazolo[3,4-b]pyridine- 7-yl]methyl]benzoic acid |
| 13 | 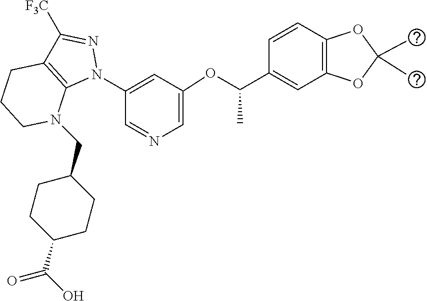 [SMILES: C[C@H](Oc1cncc(-n2nc(C(F)(F)F)c3c2N(C[C@H]2CC[C@H](C(=O)O)CC2)CCC3)c1)c1ccc2c(c1)OC(C)(C)O2] | Trans-4-[[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- yl]methyl]cyclohexanecarboxylic acid |
| 14 | 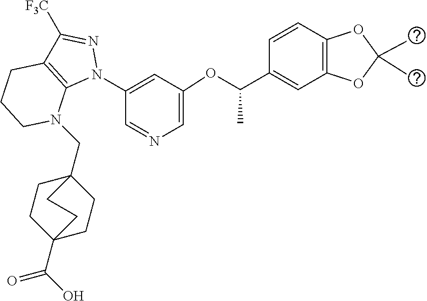 [SMILES: C[C@H](Oc1cncc(-n2nc(C(F)(F)F)c3c2N(CC24CCC(C(=O)O)(CC2)CC4)CCC3)c1)c1ccc2c(c1)OC(C)(C)O2] | 4-[[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- yl]methyl]bicyclo[2.2.2]octane-1- carboxylic acid |
| 15 | 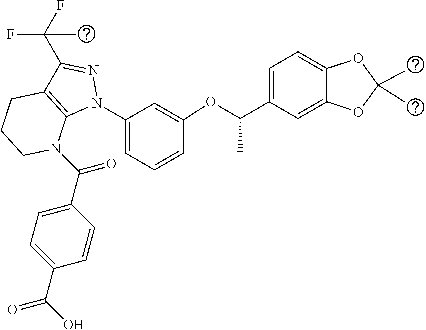 [SMILES: C[C@H](Oc1cccc(-n2nc(C(C)(F)F)c3c2N(C(=O)c2ccc(C(=O)O)cc2)CCC3)c1)c1ccc2c(c1)OC(C)(C)O2] | 4-[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- carbonyl]benzoic acid |
| 16 | 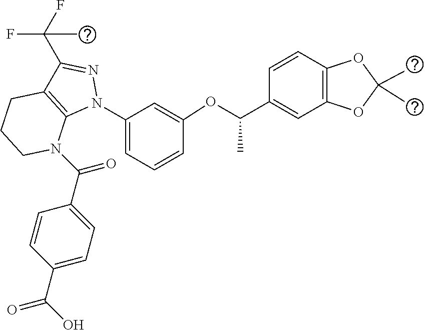 [SMILES: C[C@H](Oc1cccc(-n2nc(C(C)(F)F)c3c2N(C(=O)c2ccc(C(=O)O)cc2)CCC3)c1)c1ccc2c(c1)OC(C)(C)O2] | 4-[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- carbonyl]benzoic acid |
| 17 | 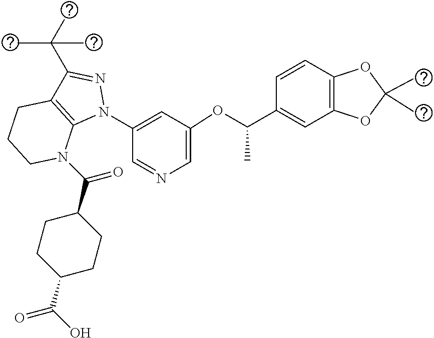 [SMILES: C[C@H](Oc1cncc(-n2nc(C(C)(C)C)c3c2N(C(=O)[C@H]2CC[C@H](C(=O)O)CC2)CCC3)c1)c1ccc2c(c1)OC(C)(C)O2] | Trans-4-[1-[5-[(1S)-1-(2,2-difluoro-1,3- benzodioxol-5-yl)ethoxy]-3-pyridyl]-3- (trifluoromethyl)-5,6-dihydro-4H- pyrazolo[3,4-b]pyridine-7- carbonyl]cyclohexanecarboxylic acid |
| 18 | 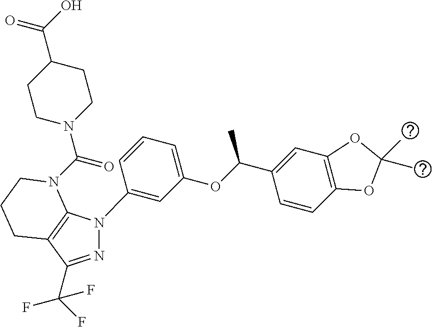 [SMILES: C[C@H](Oc1cccc(-n2nc(C(F)(F)F)c3c2N(C(=O)N2CCC(C(=O)O)CC2)CCC3)c1)c1ccc2c(c1)OC(C)(C)O2] | 1-[1-[3-[(1S)-1-(2,2-difluoro-1,3- benzodioxol-5-yl)ethoxy]phenyl]-3- (trifluoromethyl)-5,6-dihydro-4H- pyrazolo[3,4-b]pyridine-7- carbonyl]piperidine-4-carboxylic acid |
| 19 | 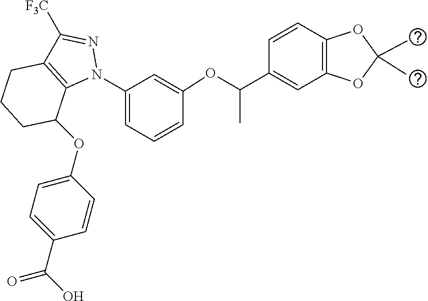 [SMILES: CC(Oc1cccc(-n2nc(C(F)(F)F)c3c2C(Oc2ccc(C(=O)O)cc2)CCC3)c1)c1ccc2c(c1)OC(C)(C)O2] | 4-[[1-[3-[1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 20 | 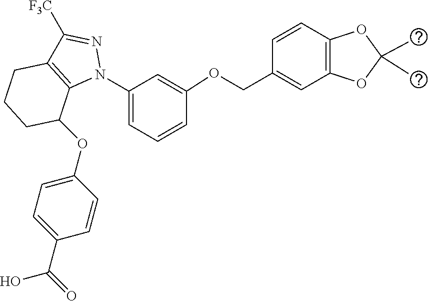 [SMILES: CC1(C)Oc2ccc(COc3cccc(-n4nc(C(F)(F)F)c5c4C(Oc4ccc(C(=O)O)cc4)CCC5)c3)cc2O1] | 4-[[1-[3-[(2,2-difluoro-1,3-benzodioxol- 5-yl)methoxyphenyl]-3- (trifluoromethyl)-4,5,6,7- tetrahydroindazol-7-yl]oxy]benzoic acid |
| 21 | 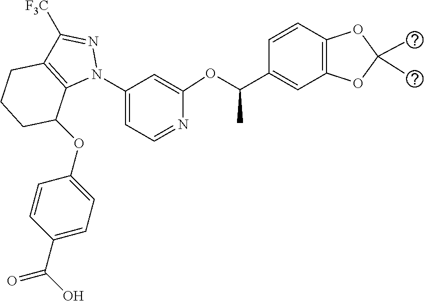 [SMILES: C[C@@H](Oc1cc(-n2nc(C(F)(F)F)c3c2C(Oc2ccc(C(=O)O)cc2)CCC3)ccn1)c1ccc2c(c1)OC(C)(C)O2] | 4-[[1-[2-[(1R)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 22 | 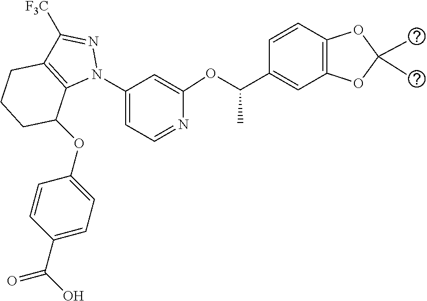 [SMILES: C[C@H](Oc1cc(-n2nc(C(F)(F)F)c3c2C(Oc2ccc(C(=O)O)cc2)CCC3)ccn1)c1ccc2c(c1)OC(C)(C)O2] | 4-[[1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 23 | 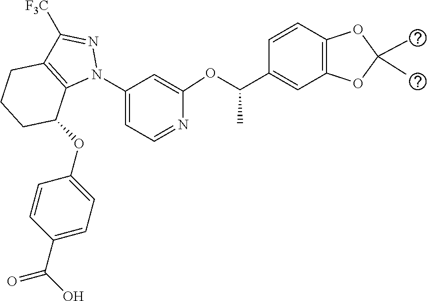 [SMILES: C[C@H](Oc1cc(-n2nc(C(F)(F)F)c3c2[C@H](Oc2ccc(C(=O)O)cc2)CCC3)ccn1)c1ccc2c(c1)OC(C)(C)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 24 | 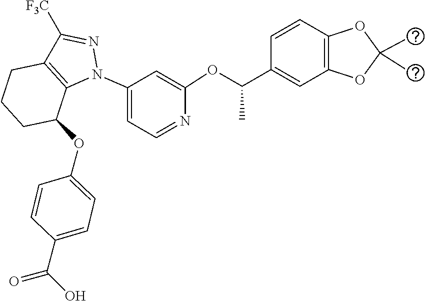 [SMILES: C[C@H](Oc1cc(-n2nc(C(F)(F)F)c3c2[C@@H](Oc2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]oxy]benzoic acid |
| 25 | 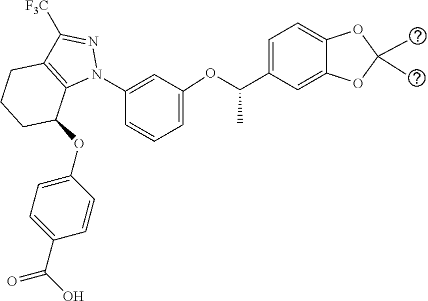 [SMILES: C[C@H](Oc1cccc(-n2nc(C(F)(F)F)c3c2[C@@H](Oc2ccc(C(=O)O)cc2)CCC3)c1)c1ccc2c(c1)OC(C)(C)O2] | 4-[[(7S)-1-[3-[(1S)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 26 | 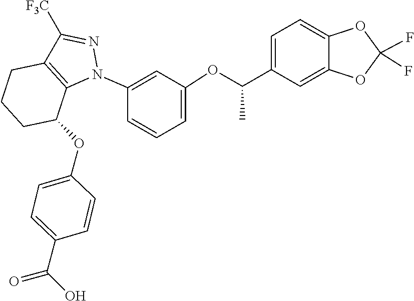 [SMILES: C[C@H](Oc1cccc(-n2nc(C(F)(F)F)c3c2[C@H](Oc2ccc(C(=O)O)cc2)CCC3)c1)c1ccc2c(c1)OC(F)(F)O2] | 4-[[(7R)-1-[3-[(1S)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 27 | 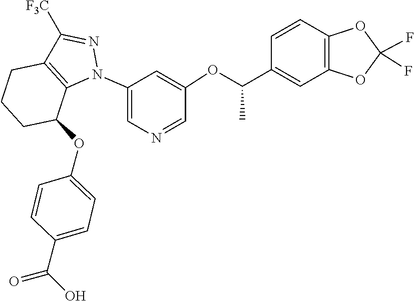 [SMILES: C[C@H](Oc1cncc(-n2nc(C(F)(F)F)c3c2[C@@H](Oc2ccc(C(=O)O)cc2)CCC3)c1)c1ccc2c(c1)OC(F)(F)O2] | 4-[[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 28 | 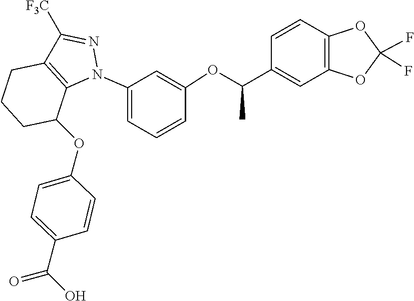 [SMILES: C[C@@H](Oc1cccc(-n2nc(C(F)(F)F)c3c2C(Oc2ccc(C(=O)O)cc2)CCC3)c1)c1ccc2c(c1)OC(F)(F)O2] | 4-[[1-[3-[(1R)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 29 | 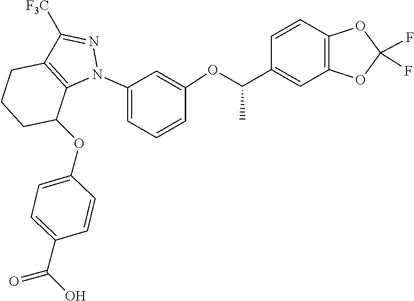 [SMILES: C[C@H](Oc1cccc(-n2nc(C(F)(F)F)c3c2C(Oc2ccc(C(=O)O)cc2)CCC3)c1)c1ccc2c(c1)OC(F)(F)O2] | 4-[[1-[3-[(1S)-1-(2,2-difluoro-1,3- benzodioxol-5-yl)ethoxy]phenyl]-3- (trifluoromethyl)-4,5,6,7- tetrahydroindazol-7-yl]oxy]benzoic acid |
| 30 | 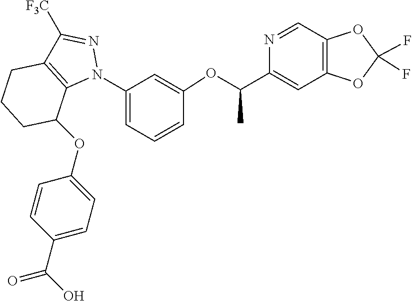 [SMILES: C[C@@H](Oc1cccc(-n2nc(C(F)(F)F)c3c2C(Oc2ccc(C(=O)O)cc2)CCC3)c1)c1cc2c(cn1)OC(F)(F)O2] | 4-[[1-[3-[(1R)-1-(2,2-difluoro- [1,3]dioxolo[4,5-c]pyridine-6- yl)ethoxy]phenyl]-3-(trifluoromethyl)- 4,5,6,7-tetrahydroindazol-7- yl]oxy]benzoic acid |
| 31 | 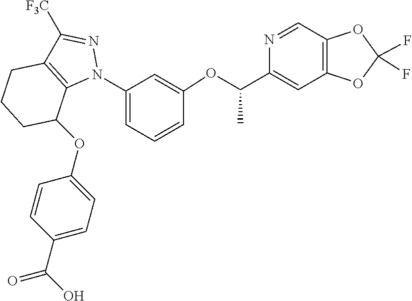 [SMILES: C[C@H](Oc1cccc(-n2nc(C(F)(F)F)c3c2C(Oc2ccc(C(=O)O)cc2)CCC3)c1)c1cc2c(cn1)OC(F)(F)O2] | 4-[[1-[3-[(1S)-1-(2,2-difluoro- [1,3]dioxolo[4,5-c]pyridine-6- yl)ethoxy]phenyl]-3-(trifluoromethyl)- 4,5,6,7-tetrahydroindazol-7- yl]oxy]benzoic acid |
| 32 | 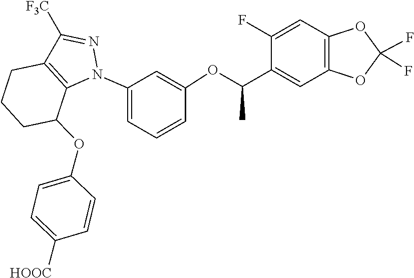 [SMILES: C[C@@H](Oc1cccc(-n2nc(C(F)(F)F)c3c2C(Oc2ccc(C(=O)O)cc2)CCC3)c1)c1cc2c(cc1F)OC(F)(F)O2] | 4-[[1-[3-[(1R)-1-(2,2,6-trifluoro-1,3- benzodioxol-5-yl)ethoxy]phenyl]-3- (trifluoromethyl)-4,5,6,7- tetrahydroindazol-7-yl]oxy]benzoic acid |
| 33 | 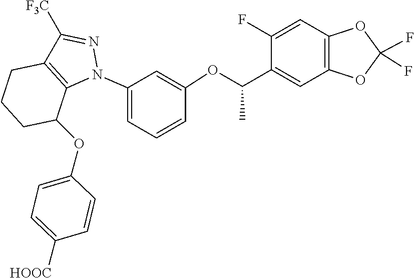 [SMILES: C[C@H](Oc1cccc(-n2nc(C(F)(F)F)c3c2C(Oc2ccc(C(=O)O)cc2)CCC3)c1)c1cc2c(cc1F)OC(F)(F)O2] | 4-[1-[3-[(1S)-1-(2,2,6-trifluoro-1,3- benzodioxol-5-yl)ethoxy]phenyl]-3- (trifluoromethyl)-4,5,6,7- tetrahydroindazol-7-yl]oxy]benzoic acid |
| 34 | 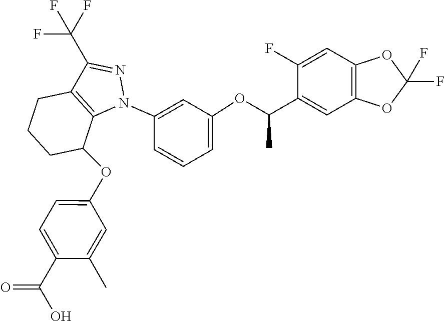 [SMILES: Cc1cc(OC2CCCc3c(C(F)(F)F)nn(-c4cccc(O[C@H](C)c5cc6c(cc5F)OC(F)(F)O6)c4)c32)ccc1C(=O)O] | 2-methyl-4-[[1-[3-[(1R)-1-(2,2,6- trifluoro-1,3-benzodioxol-5- yl)ethoxy phenyl]-3-(trifluoromethyl)- 4,5,6,7-tetrahydroindazol-7- yl]oxy]benzoic acid |
| 35 | 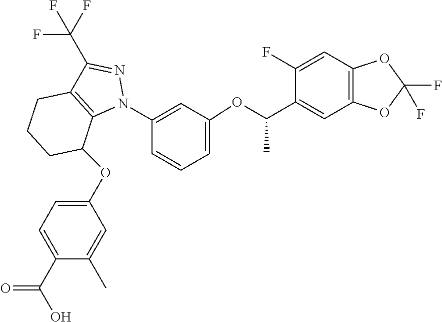 [SMILES: Cc1cc(OC2CCCc3c(C(F)(F)F)nn(-c4cccc(O[C@@H](C)c5cc6c(cc5F)OC(F)(F)O6)c4)c32)ccc1C(=O)O] | 2-methyl-4-[[1-[3-[(1S)-1 -(2,2,6- trifluoro-1,3-benzodioxol-5- yl)ethoxy]phenyl]-3-(trifluoromethyl)- 4,5,6,7-tetrahydroindazol-7- yl]oxy]benzoic acid |
| 36 | 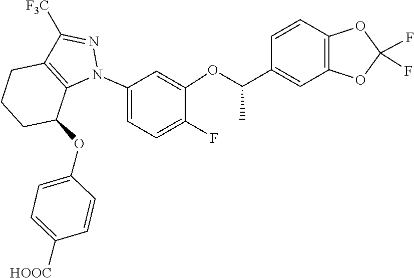 [SMILES: C[C@H](Oc1cc(-n2nc(C(F)(F)F)c3c2[C@@H](Oc2ccc(C(=O)O)cc2)CCC3)ccc1F)c1ccc2c(c1)OC(F)(F)O2] | 4-[[(7S)-1-[3-[(1S)-1-(2,2-difluoro-1,3- benzodioxol-5-yl)ethoxy]-4-fluoro- phenyl]-3-(trifluoromethyl)-4,5,6,7- tetrahydroindazol-7-yl]oxy]benzoic acid |
| 36a | 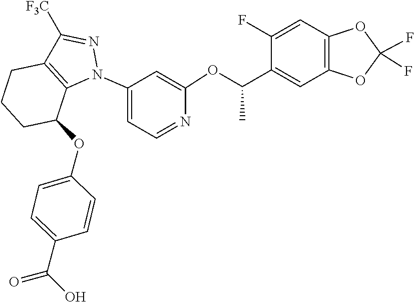 [SMILES: C[C@H](Oc1cc(-n2nc(C(F)(F)F)c3c2[C@@H](Oc2ccc(C(=O)O)cc2)CCC3)ccn1)c1cc2c(cc1F)OC(F)(F)O2] | 4-[[(7S)-1-[2-[(1S)-1-(2,2,6-trifluoro- 1,3-benzodioxol-5-yl)ethoxy]-4- pyridyl]-3-(trifluoromethyl)-4,5,6,7- tetrahydroindazol-7-yl]oxy]benzoic acid |
| 36b | 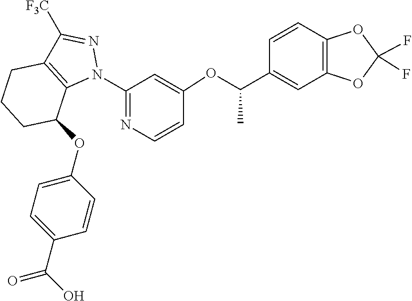 [SMILES: C[C@H](Oc1ccnc(-n2nc(C(F)(F)F)c3c2[C@@H](Oc2ccc(C(=O)O)cc2)CCC3)c1)c1ccc2c(c1)OC(F)(F)O2] | 4-[[(7S)-1-[4-[(1S)-1-(2,2-difluoro-1,3- benzodioxol-5-yl)ethoxy]-2-pyridyl]-3- (trifluoromethyl)-4,5,6,7- tetrahydroindazol-7-yl]oxy]benzoic acid |
| 36c | 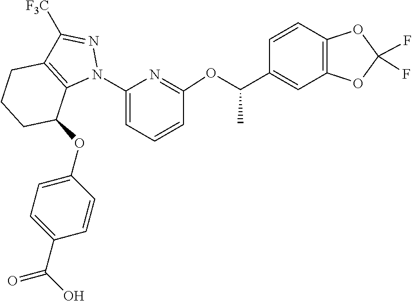 [SMILES: C[C@H](Oc1cccc(-n2nc(C(F)(F)F)c3c2[C@@H](Oc2ccc(C(=O)O)cc2)CCC3)n1)c1ccc2c(c1)OC(F)(F)O2] | 4-[[(7S)-1-[6-[(1S)-1-(2,2-difluoro-1,3- benzodioxol-5-yl)ethoxy]-2-pyridyl]-3- (trifluoromethyl)-4,5,6,7- tetrahydroindazol-7-yl]oxy]benzoic acid |
| 36d | 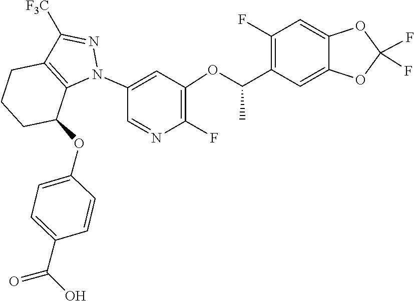 [SMILES: C[C@H](Oc1cc(-n2nc(C(F)(F)F)c3c2[C@@H](Oc2ccc(C(=O)O)cc2)CCC3)cnc1F)c1cc2c(cc1F)OC(F)(F)O2] | 4-[[(7S)-1-[6-fluoro-5-[(1S)-1-(2,2,6- trifluoro-1,3-benzodioxol-5-yl)ethoxy]- 3-pyridyl]-3-(trifluoromethyl)-4,5,6,7- tetrahydroindazol-7-yl]oxy]benzoic acid |
| 36e | 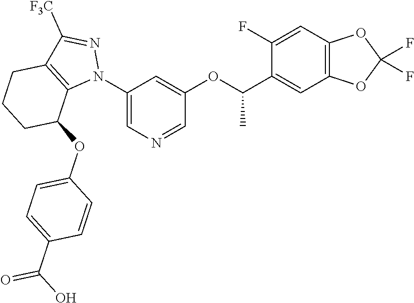 [SMILES: C[C@H](Oc1cncc(-n2nc(C(F)(F)F)c3c2[C@@H](Oc2ccc(C(=O)O)cc2)CCC3)c1)c1cc2c(cc1F)OC(F)(F)O2] | 4-[[(7S)-1-[5-[(1S)-1-(2,2,6-trifluoro- 1,3-benzodioxol-5-yl)ethoxy]-3- pyridyl]-3-(trifluoromethyl)-4,5,6,7- tetrahydroindazol-7-yl]oxy]benzoic acid |
| 36f | 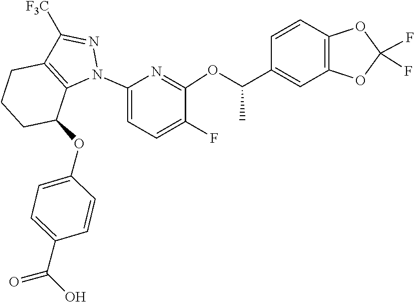 [SMILES: C[C@H](Oc1nc(-n2nc(C(F)(F)F)c3c2[C@@H](Oc2ccc(C(=O)O)cc2)CCC3)ccc1F)c1ccc2c(c1)OC(F)(F)O2] | 4-[[(7S)-1-[6-[(1S)-1-(2,2-difluoro-1,3- benzodioxol-5-yl)ethoxy]-5-fluoro-2- pyridyl]-3-(trifluoromethyl)-4,5,6,7- tetrahydroindazol-7-yl]oxy]benzoic acid |
| 36g | 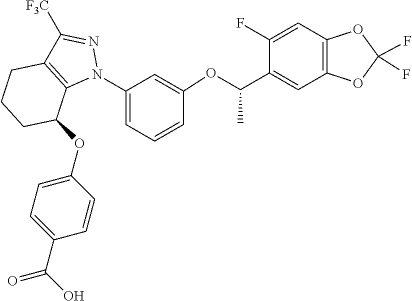 [SMILES: C[C@H](Oc1cccc(-n2nc(C(F)(F)F)c3c2[C@@H](Oc2ccc(C(=O)O)cc2)CCC3)c1)c1cc2c(cc1F)OC(F)(F)O2] | 4-[[(7S)-1-[3-[(1S)-1-(2,2,6-trifluoro- 1,3-benzodioxol-5-yl)ethoxy]phenyl]-3- (trifluoromethyl)-4,5,6,7- tetrahydroindazol-7-yl]oxy]benzoic acid |
| 36h | 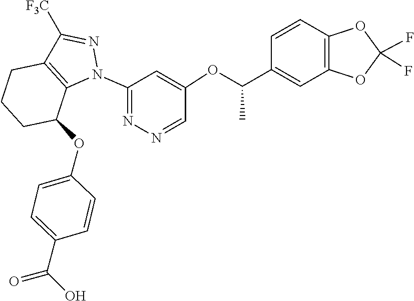 [SMILES: C[C@H](Oc1cnnc(-n2nc(C(F)(F)F)c3c2[C@@H](Oc2ccc(C(=O)O)cc2)CCC3)c1)c1ccc2c(c1)OC(F)(F)O2] | 4-[[(7S)-1-[5-[(1S)-1-(2,2-difluoro-1,3- benzodioxol-5-yl)ethoxy]pyridazin-3- yl]-3-(trifluoromethyl)-4,5,6,7- tetrahydroindazol-7-yl]oxy]benzoic acid |
| 36i | 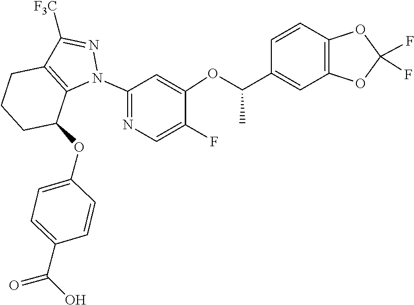 [SMILES: C[C@H](Oc1cc(-n2nc(C(F)(F)F)c3c2[C@@H](Oc2ccc(C(=O)O)cc2)CCC3)ncc1F)c1ccc2c(c1)OC(F)(F)O2] | 4-[[(7S)-1-[4-[(1S)-1-(2,2-difluoro-1,3- benzodioxol-5-yl)ethoxy]-5-fluoro-2- pyridyl]-3-(trifluoromethyl)-4,5,6,7- tetrahydroindazol-7-yl]oxy]benzoic acid |
| 36j | 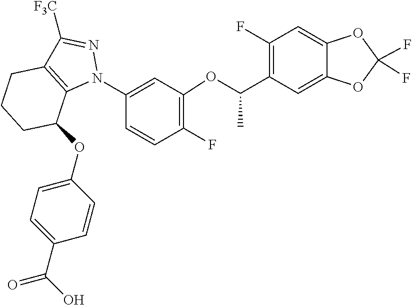 [SMILES: C[C@H](Oc1cc(-n2nc(C(F)(F)F)c3c2[C@@H](Oc2ccc(C(=O)O)cc2)CCC3)ccc1F)c1cc2c(cc1F)OC(F)(F)O2] | 4-[[(7S)-1-[4-fluoro-3-[(1S)-1-(2,2,6- trifluoro-1,3-benzodioxol-5- yl)ethoxy]phenyl]-3-(trifluoromethyl)- 4,5,6,7-tetrahydroindazol-7- yl]oxy]benzoic acid |
| 36k | 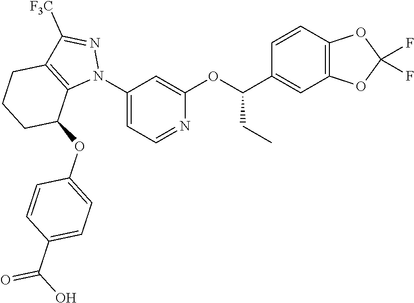 [SMILES: CC[C@H](Oc1cc(-n2nc(C(F)(F)F)c3c2[C@@H](Oc2ccc(C(=O)O)cc2)CCC3)ccn1)c1ccc2c(c1)OC(F)(F)O2] | 4-[[(7S)-1-[2-[(1S)-1-(2,2-difluoro-1,3- benzodioxol-5-yl)propoxy]-4-pyridyl]-3- (trifluoromethyl)-4,5,6,7- tetrahydroindazol-7-yl]oxy]benzoic acid |
| 36l | 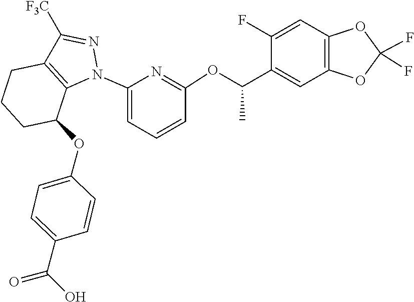 [SMILES: C[C@H](Oc1cccc(-n2nc(C(F)(F)F)c3c2[C@@H](Oc2ccc(C(=O)O)cc2)CCC3)n1)c1cc2c(cc1F)OC(F)(F)O2] | 4-[[(7S)-1-[6-[(1S)-1-(2,2,6-trifluoro- 1,3-benzodioxol-5-yl)ethoxy]-2- pyridyl]-3-(trifluoromethyl)-4,5,6,7- tetrahydroindazol-7-yl]oxy]benzoic acid |
| 36m | 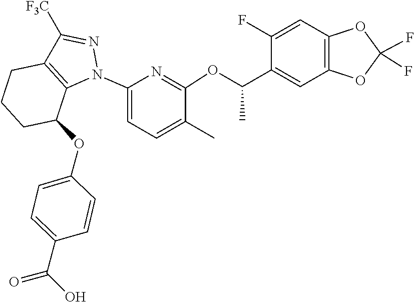 [SMILES: Cc1ccc(-n2nc(C(F)(F)F)c3c2[C@@H](Oc2ccc(C(=O)O)cc2)CCC3)nc1O[C@@H](C)c1cc2c(cc1F)OC(F)(F)O2] | 4-[[(7S)-1-[5-fluoro-6-[(1S)-1-(2,2,6- trifluoro-1,3-benzodioxol-5-yl)ethoxy]- 2-pyridyl]-3-(trifluoromethyl)-4,5,6,7- tetrahydroindazol-7-yl]oxy]benzoic acid |
| 37 | 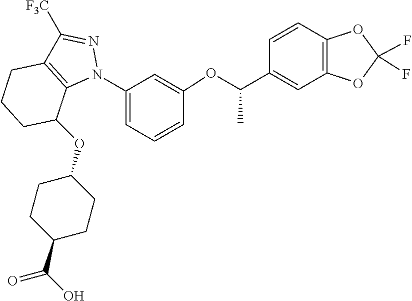 [SMILES: C[C@H](Oc1cccc(-n2nc(C(F)(F)F)c3c2C(O[C@H]2CC[C@H](C(=O)O)CC2)CCC3)c1)c1ccc2c(c1)OC(F)(F)O2] | 4-[[1-[3-[(1S)-1-(2,2-difluoro-1,3- benzodioxol-5-yl)ethoxyphenyl]-3- (trifluoromethyl)-4,5,6,7- tetrahydroindazol-7- yl]oxy]cyclohexanecarboxylic acid |
| 38 | 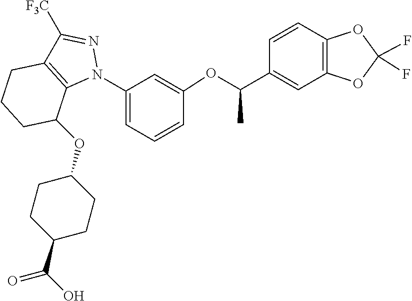 [SMILES: C[C@@H](Oc1cccc(-n2nc(C(F)(F)F)c3c2C(O[C@H]2CC[C@H](C(=O)O)CC2)CCC3)c1)c1ccc2c(c1)OC(F)(F)O2] | 4-[[1-[3-[(1R)-1-(2,2-difluoro-1,3- benzodioxol-5-yl)ethoxyphenyl]-3- (trifluoromethyl)-4,5,6,7- tetrahydroindazol-7- yl]oxy]cyclohexanecarboxylic acid |
| 39 | 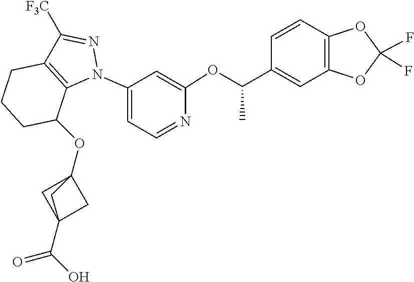 [SMILES: C[C@H](Oc1cc(-n2nc(C(F)(F)F)c3c2C(OC24CC(C(=O)O)(C2)C4)CCC3)ccn1)c1ccc2c(c1)OC(F)(F)O2] | 3-[[1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 40 | 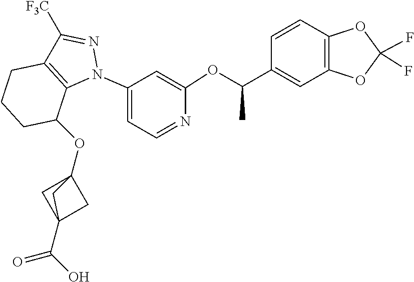 [SMILES: C[C@@H](Oc1cc(-n2nc(C(F)(F)F)c3c2C(OC24CC(C(=O)O)(C2)C4)CCC3)ccn1)c1ccc2c(c1)OC(F)(F)O2] | 3-[[1-[2-[(1R)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 41 | 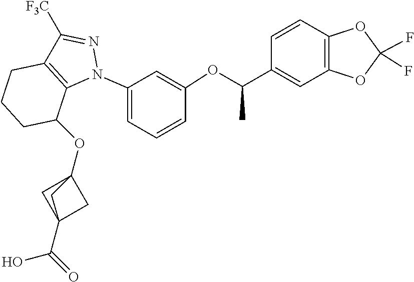 [SMILES: C[C@@H](Oc1cccc(-n2nc(C(F)(F)F)c3c2C(OC24CC(C(=O)O)(C2)C4)CCC3)c1)c1ccc2c(c1)OC(F)(F)O2] | 3-[[1-[3-[(1R)-1-(2,2-difluoro-1,3- benzodioxol-5-yl)ethoxylphenyl]-3- (trifluoromethyl)-4,5,6,7- tetrahydroindazol-7- yl]oxy]bicyclo[1.1.1]pentane-1- carboxylic acid |
| 42 | 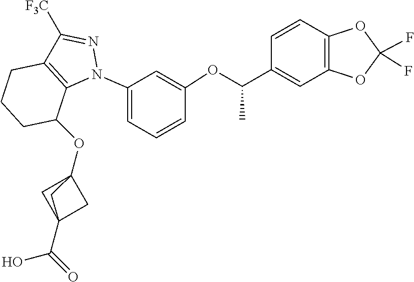 [SMILES: C[C@H](Oc1cccc(-n2nc(C(F)(F)F)c3c2C(OC24CC(C(=O)O)(C2)C4)CCC3)c1)c1ccc2c(c1)OC(F)(F)O2] | 3-[[1-[3-[(1S)-1-(2,2-difluoro-1,3- benzodioxol-5-yl)ethoxy]phenyl]-3- (trifluoromethyl)-4,5,6,7- tetrahydroindazol-7- yl]oxy]bicyclo[1.1.1]pentane-1- carboxylic acid |
| 43 | 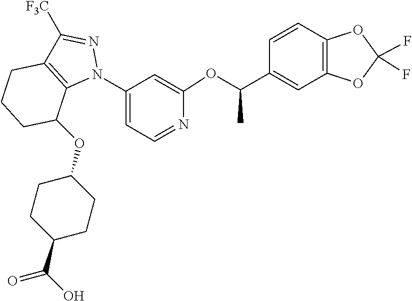 [SMILES: C[C@@H](Oc1cc(-n2nc(C(F)(F)F)c3c2C(O[C@H]2CC[C@H](C(=O)O)CC2)CCC3)ccn1)c1ccc2c(c1)OC(F)(F)O2] | Trans-4-[1-[2-[(1R)-1-(2,2-difluoro-1,3- benzodioxol-5-yl)ethoxy]-4-pyridyl]-3- (trifluoromethyl)-4,5,6,7- tetrahydroindazol-7- yl]oxy]cyclohexanecarboxylic acid |
| 44 | 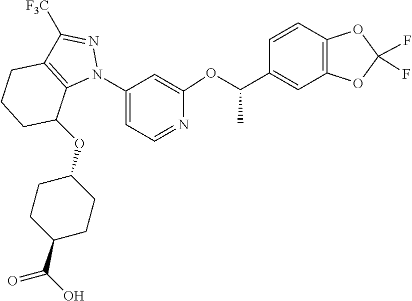 [SMILES: C[C@H](Oc1cc(-n2nc(C(F)(F)F)c3c2C(O[C@H]2CC[C@H](C(=O)O)CC2)CCC3)ccn1)c1ccc2c(c1)OC(F)(F)O2] | Trans-4-[1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]cyclohexanecarboxylic acid |
| 45 | 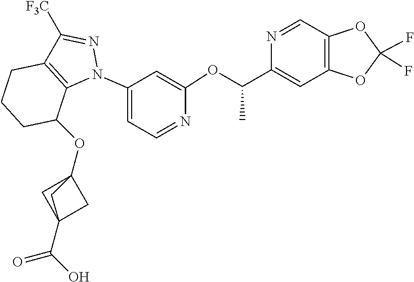 [SMILES: C[C@H](Oc1cc(-n2nc(C(F)(F)F)c3c2C(OC24CC(C(=O)O)(C2)C4)CCC3)ccn1)c1cc2c(cn1)OC(F)(F)O2] | 3-[[1-[2-[(1S)-1-(2,2-difluoro- [1,3]dioxolo[4,5-c]pyridin-6-yl)ethoxy]- 4-pyridyl]-3-(trifluoromethyl)-4,5,6,7- tetrahydroindazol-7- yl]oxy]bicyclo[1.1.1]pentane-1- carboxylic acid |
| 46 | 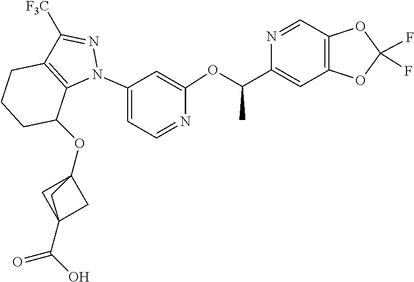 [SMILES: C[C@@H](Oc1cc(-n2nc(C(F)(F)F)c3c2C(OC24CC(C(=O)O)(C2)C4)CCC3)ccn1)c1cc2c(cn1)OC(F)(F)O2] | 3-[[1-[2-[(1R)-1-(2,2-difluoro- [1,3]dioxolo[4,5-c]pyridin-6-yl)ethoxy]- 4-pyridyl]-3-(trifluoromethyl)-4,5,6,7- tetrahydroindazol-7- yl]oxy]bicyclo[1.1.1]pentane-1- carboxylic acid |
| 47 | 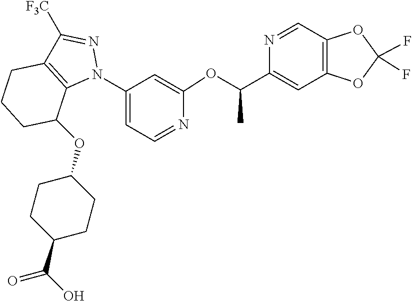 [SMILES: C[C@@H](Oc1cc(-n2nc(C(F)(F)F)c3c2C(O[C@H]2CC[C@H](C(=O)O)CC2)CCC3)ccn1)c1cc2c(cn1)OC(F)(F)O2] | Trans-4-[[1-[2-[(1R)-1-(2,2-difluoro- [1,3]dioxolo[4,5-c]pyridin-6-yl)ethoxy]- 4-pyridyl]-3-(trifluoromethyl)-4,5,6,7- tetrahydroindazol-7- yl]oxy]cyclohexanecarboxylic acid |
| 48 | 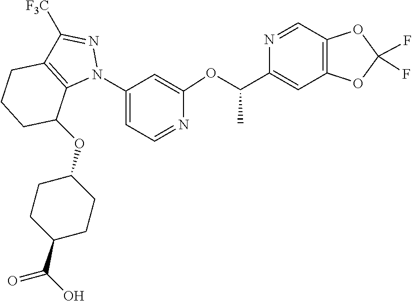 [SMILES: C[C@H](Oc1cc(-n2nc(C(F)(F)F)c3c2C(O[C@H]2CC[C@H](C(=O)O)CC2)CCC3)ccn1)c1cc2c(cn1)OC(F)(F)O2] | Trans-4-[1-[2-[(1S)-1-(2,2-difluoro- [1,3]dioxolo[4,5-c]pyridin-6-yl)ethoxy]- 4-pyridyl]-3-(trifluoromethyl)-4,5,6,7- tetrahydroindazol-7- yl]oxy]cyclohexanecarboxylic acid |
| 49 | 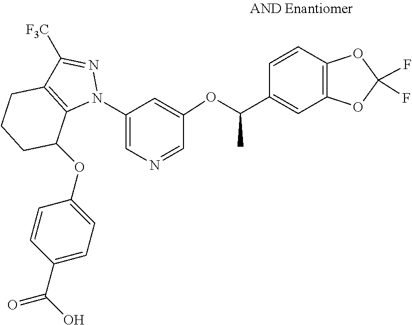 [SMILES: C[C@@H](Oc1cncc(-n2nc(C(F)(F)F)c3c2C(Oc2ccc(C(=O)O)cc2)CCC3)c1)c1ccc2c(c1)OC(F)(F)O2] | 4-[[1-[5-[rac-(1R)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 50 | 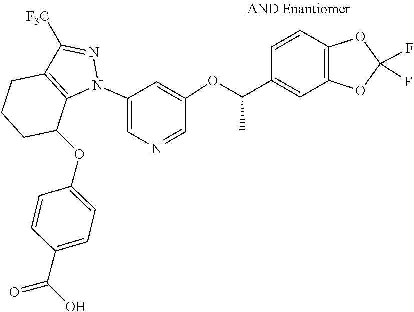 [SMILES: C[C@H](Oc1cncc(-n2nc(C(F)(F)F)c3c2C(Oc2ccc(C(=O)O)cc2)CCC3)c1)c1ccc2c(c1)OC(F)(F)O2] | 4-[[1-[5-[rac-(1S)-1-(2,2-difluoro-1,3- benzodioxol-5-yl)ethoxy]-3-pyridyl]-3- (trifluoromethyl)-4,5,6,7- tetrahydroindazol-7-yl]oxy]benzoic acid |
| 51 | 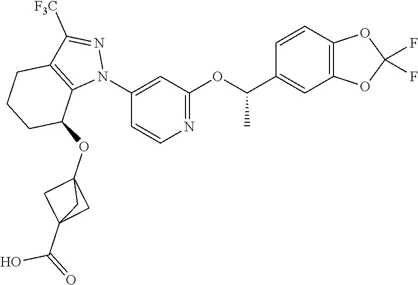 [SMILES: C[C@H](Oc1cc(-n2nc(C(F)(F)F)c3c2[C@@H](OC24CC(C(=O)O)(C2)C4)CCC3)ccn1)c1ccc2c(c1)OC(F)(F)O2] | 3-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 52 | 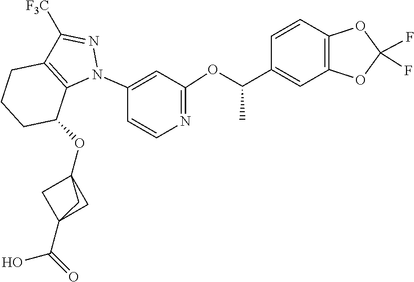 [SMILES: C[C@H](Oc1cc(-n2nc(C(F)(F)F)c3c2[C@H](OC24CC(C(=O)O)(C2)C4)CCC3)ccn1)c1ccc2c(c1)OC(F)(F)O2] | 3-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[1.1.1]pentane-1- carboxylic acid |
| 53 | 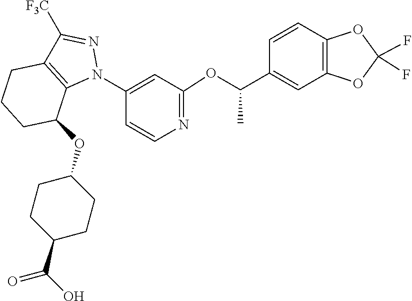 [SMILES: C[C@H](Oc1cc(-n2nc(C(F)(F)F)c3c2[C@@H](O[C@H]2CC[C@H](C(=O)O)CC2)CCC3)ccn1)c1ccc2c(c1)OC(F)(F)O2] | Trans-4-[[(7S)-1-[2-[(1S)-1-(2,2- difluoro-1,3-benzodioxol-5-yl)ethoxy]- 4-pyridyl]-3-(trifluoromethyl)-4,5,6,7- tetrahydroindazol-7- yl]oxy]cyclohexanecarboxylic acid |
| 54 | 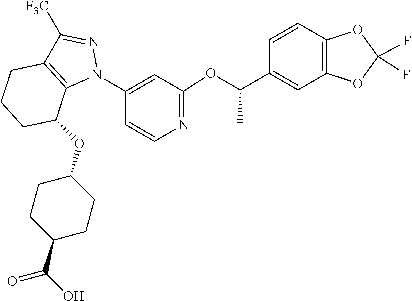 [SMILES: C[C@H](Oc1cc(-n2nc(C(F)(F)F)c3c2[C@H](O[C@H]2CC[C@H](C(=O)O)CC2)CCC3)ccn1)c1ccc2c(c1)OC(F)(F)O2] | Trans-4-[[(7R)-1-[2-[(1S)-1-(2,2- difluoro-1,3-benzodioxol-5-yl)ethoxy]- 4-pyridyl]-3-(trifluoromethyl)-4,5,6,7- tetrahydroindazol-7- yl]oxy]cyclohexanecarboxylic acid |
| 55 | 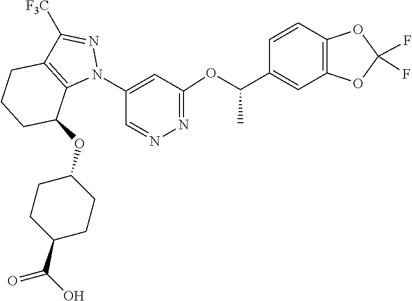 [SMILES: C[C@H](Oc1cc(-n2nc(C(F)(F)F)c3c2[C@@H](O[C@H]2CC[C@H](C(=O)O)CC2)CCC3)cnn1)c1ccc2c(c1)OC(F)(F)O2] | Trans-4-[[(7S)-1-[6-[(1S)-1-(2,2- difluoro-1,3-benzodioxol-5- yl)ethoxy]pyridazin-4-yl]-3- (trifluoromethyl)-4,5,6,7- tetrahydroindazol-7- yl]oxy]cyclohexanecarboxylic acid |
| 56 | 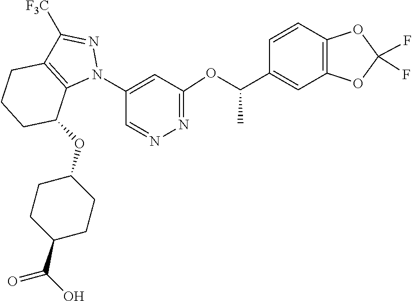 [SMILES: C[C@H](Oc1cc(-n2nc(C(F)(F)F)c3c2[C@H](O[C@H]2CC[C@H](C(=O)O)CC2)CCC3)cnn1)c1ccc2c(c1)OC(F)(F)O2] | Trans-4-[[(7R)-1-[6-[(1S)-1-(2,2- difluoro-1,3-benzodioxol-5- yl)ethoxy]pyridazin-4-yl]-3- (trifluoromethyl)-4,5,6,7- tetrahydroindazol-7- yl]oxy]cyclohexanecarboxylic acid |
| 57 | 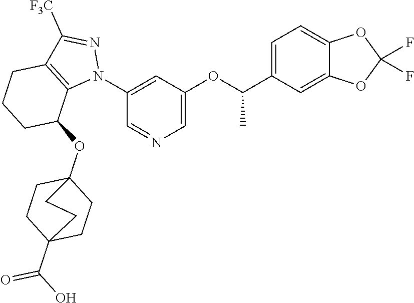 [SMILES: C[C@H](Oc1cncc(-n2nc(C(F)(F)F)c3c2[C@@H](OC24CCC(C(=O)O)(CC2)CC4)CCC3)c1)c1ccc2c(c1)OC(F)(F)O2] | 4-[[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- y]loxy]bicyclo[2.2.2]octane-1- carboxylic acid |
| 58 | 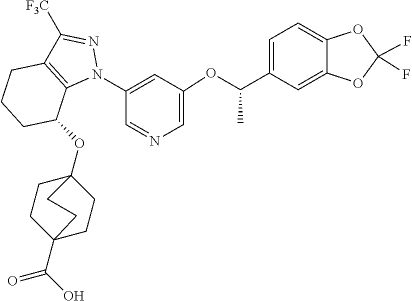 [SMILES: C[C@H](Oc1cncc(-n2nc(C(F)(F)F)c3c2[C@H](OC24CCC(C(=O)O)(CC2)CC4)CCC3)c1)c1ccc2c(c1)OC(F)(F)O2] | 4-[[(7R)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl]oxy]bicyclo[2.2.2]octane-1- carboxylic acid |
| 59 | 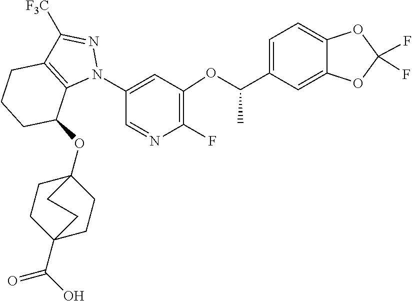 [SMILES: C[C@H](Oc1cc(-n2nc(C(F)(F)F)c3c2[C@@H](OC24CCC(C(=O)O)(CC2)CC4)CCC3)cnc1F)c1ccc2c(c1)OC(F)(F)O2] | 4-[[(7S)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7- yl]oxy]bicyclo[2.2.2]octane-1- carboxylic acid |
| 60 | 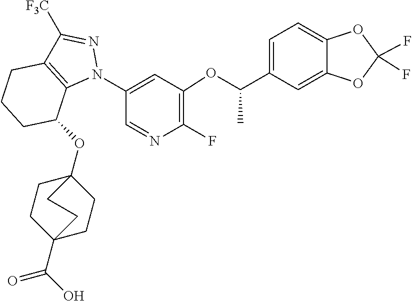 [SMILES: C[C@H](Oc1cc(-n2nc(C(F)(F)F)c3c2[C@H](OC24CCC(C(=O)O)(CC2)CC4)CCC3)cnc1F)c1ccc2c(c1)OC(F)(F)O2] | 4-[[(7R)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7- yl]oxy]bicyclo[2.2.2]octane-1- carboxylic acid |
| 61 | 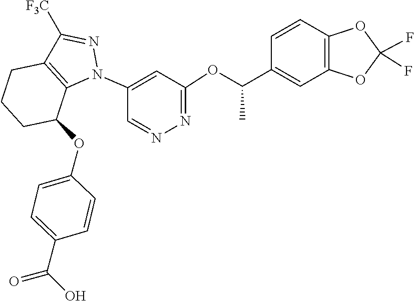 [SMILES: C[C@H](Oc1cc(-n2nc(C(F)(F)F)c3c2[C@@H](Oc2ccc(C(=O)O)cc2)CCC3)cnn1)c1ccc2c(c1)OC(F)(F)O2] | 4-[[(7S)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7-yl]oxy]benzoic acid |
| 62 | 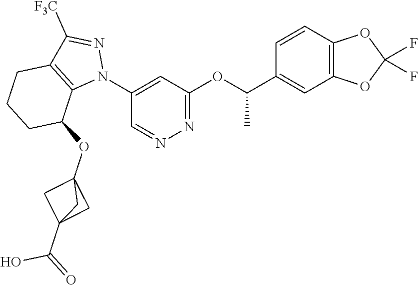 [SMILES: C[C@H](Oc1cc(-n2nc(C(F)(F)F)c3c2[C@@H](OC24CC(C(=O)O)(C2)C4)CCC3)cnn1)c1ccc2c(c1)OC(F)(F)O2] | 3-[[(7S)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7- yloxy]bicyclo[1.1.1]pentane-1- carboxylic acid |
| 63 | 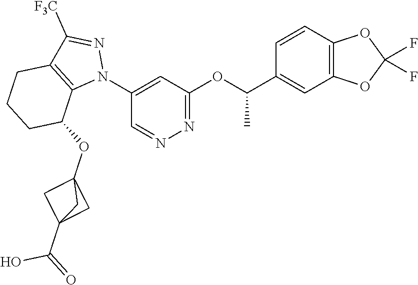 [SMILES: C[C@H](Oc1cc(-n2nc(C(F)(F)F)c3c2[C@H](OC24CC(C(=O)O)(C2)C4)CCC3)cnn1)c1ccc2c(c1)OC(F)(F)O2] | 3-[[(7R)-1-[6-[(1S)-1-(2,2-difluoro-1,3- benzodioxol-5-yl)ethoxy]pyridazin-4- yl]-3-(trifluoromethyl)-4,5,6,7- tetrahydroindazol-7- yl]oxy]bicyclo[1.1.1]pentane-1- carboxylic acid |
| 64 | 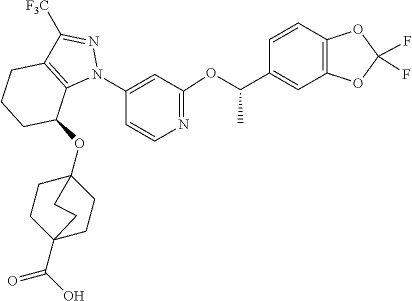 [SMILES: C[C@H](Oc1cc(-n2nc(C(F)(F)F)c3c2[C@@H](OC24CCC(C(=O)O)(CC2)CC4)CCC3)ccn1)c1ccc2c(c1)OC(F)(F)O2] | 4-[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[2.2.2]octane-1- carboxylic acid |
| 65 | 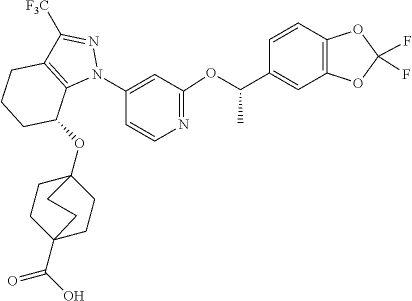 [SMILES: C[C@H](Oc1cc(-n2nc(C(F)(F)F)c3c2[C@H](OC24CCC(C(=O)O)(CC2)CC4)CCC3)ccn1)c1ccc2c(c1)OC(F)(F)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]oxy]bicyclo[2.2.2]octane-1- carboxylic acid |
| 66 | 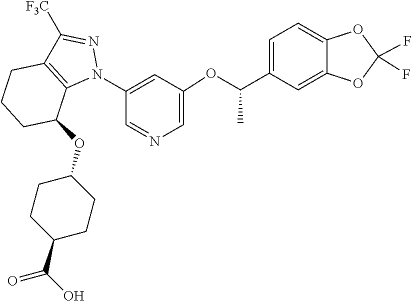 [SMILES: C[C@H](Oc1cncc(-n2nc(C(F)(F)F)c3c2[C@@H](O[C@H]2CC[C@H](C(=O)O)CC2)CCC3)c1)c1ccc2c(c1)OC(F)(F)O2] | Trans-4-[[(7S)-1-[5-[(1S)-1-(2,2- difluoro-1,3-benzodioxol-5-yl)ethoxy]- 3-pyridyl]-3-(trifluoromethyl)-4,5,6,7- tetrahydroindazol-7- yl]oxy]cyclohexanecarboxylic acid |
| 67 | 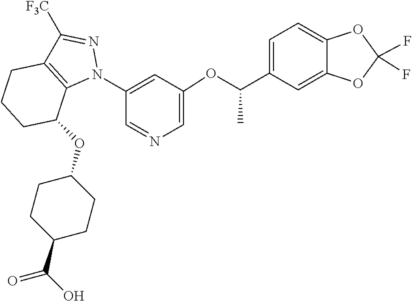 [SMILES: C[C@H](Oc1cncc(-n2nc(C(F)(F)F)c3c2[C@H](O[C@H]2CC[C@H](C(=O)O)CC2)CCC3)c1)c1ccc2c(c1)OC(F)(F)O2] | Trans-4-[[(7R)-1-[5-[(1S)-1-(2,2- difluoro-1,3-benzodioxol-5-yl)ethoxy]- 3-pyridyl]-3-(trifluoromethyl)-4,5,6,7- tetrahydroindazol-7- yl]oxy]cyclohexanecarboxylic acid |
| 68 | 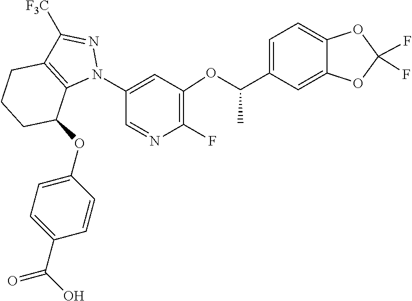 [SMILES: C[C@H](Oc1cc(-n2nc(C(F)(F)F)c3c2[C@@H](Oc2ccc(C(=O)O)cc2)CCC3)cnc1F)c1ccc2c(c1)OC(F)(F)O2] | 4-[[(7S)-1-[5-[(1S)-1-(2,2-difluoro-1,3- benzodioxol-5-yl)ethoxy]-6-fluoro-3- pyridyl]-3-(trifluoromethyl)-4,5,6,7- tetrahydroindazol-7-yl]oxy]benzoic acid |
| 69 | 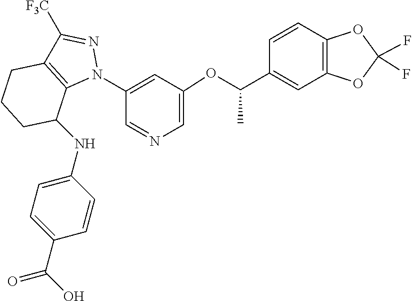 [SMILES: C[C@H](Oc1cncc(-n2nc(C(F)(F)F)c3c2C(Nc2ccc(C(=O)O)cc2)CCC3)c1)c1ccc2c(c1)OC(F)(F)O2] | 4-[[1-[5-[(1S)-1-(2,2-difluoro-1,3- benzodioxol-5-yl)ethoxy]-3-pyridyl]-3- (trifluoromethyl)-4,5,6,7- tetrahydroindazol-7-yl]amino]benzoic acid |
| 70 | 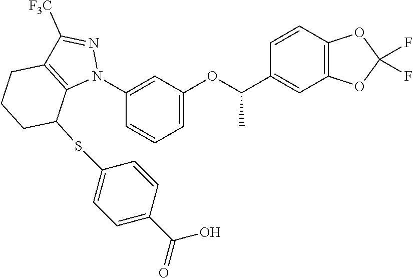 [SMILES: C[C@H](Oc1cccc(-n2nc(C(F)(F)F)c3c2C(Sc2ccc(C(=O)O)cc2)CCC3)c1)c1ccc2c(c1)OC(F)(F)O2] | 4-[[1-[3-[(1S)-1-(2,2-difluoro-1,3- benzodioxol-5-yl)ethoxy]phenyl]-3- (trifluoromethyl)-4,5,6,7- tetrahydroindazol-7-yl]sulfanyl]benzoic acid |
| 71 | 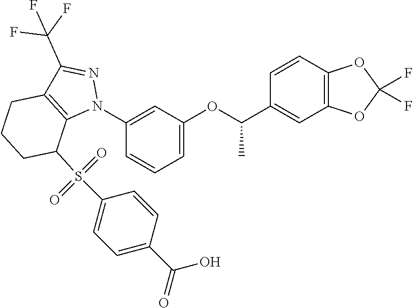 [SMILES: C[C@H](Oc1cccc(-n2nc(C(F)(F)F)c3c2C(S(=O)(=O)c2ccc(C(=O)O)cc2)CCC3)c1)c1ccc2c(c1)OC(F)(F)O2] | 4-[[1-[3-[(1S)-1-(2,2-difluoro-1,3- benzodioxol-5-yl)ethoxy]phenyl]-3- (trifluoromethyl)-4,5,6,7- tetrahydroindazol-7-yl]sulfonyl]benzoic acid |
| 72 | 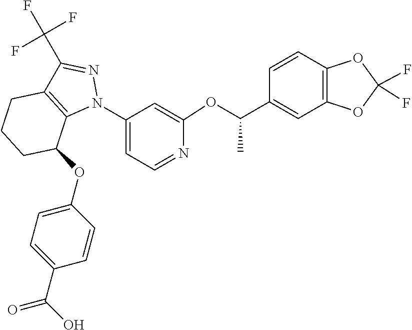 [SMILES: C[C@H](Oc1cc(-n2nc(C(F)(F)F)c3c2[C@@H](Oc2ccc(C(=O)O)cc2)CCC3)ccn1)c1ccc2c(c1)OC(F)(F)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]sulfanyl]benzoic acid |
| 73 | 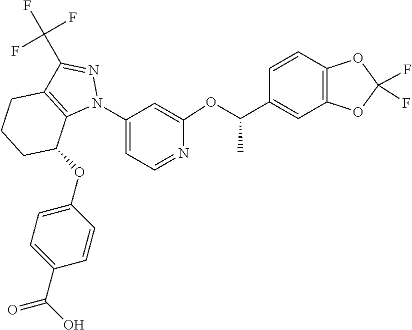 [SMILES: C[C@H](Oc1cc(-n2nc(C(F)(F)F)c3c2[C@H](Oc2ccc(C(=O)O)cc2)CCC3)ccn1)c1ccc2c(c1)OC(F)(F)O2] | 4-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]sulfanyl]benzoic acid |
| 74 (P1) | 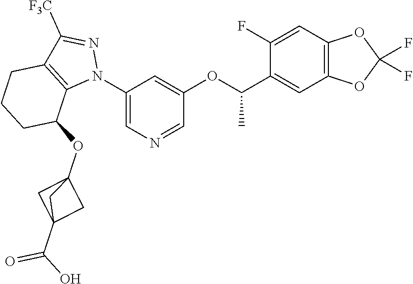 [SMILES: C[C@H](Oc1cncc(-n2nc(C(F)(F)F)c3c2[C@@H](OC24CC(C(=O)O)(C2)C4)CCC3)c1)c1cc2c(cc1F)OC(F)(F)O2] | 3-[[(7S)-1-[5-[(1S)-1-(2,2,6-trifluoro- 1,3-benzodioxol-5-yl)ethoxy]-3- pyridyl]-3-(trifluoromethyl)-4,5,6,7- tetrahydroindazol-7- yl]oxy]bicyclo[1.1.1]pentane-1- carboxylic acid |
| 74 (P2) | 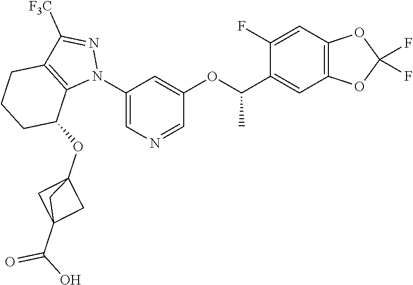 [SMILES: C[C@H](Oc1cncc(-n2nc(C(F)(F)F)c3c2[C@H](OC24CC(C(=O)O)(C2)C4)CCC3)c1)c1cc2c(cc1F)OC(F)(F)O2] | 3-[[(7R)-1-[5-[(1S)-1-(2,2,6-trifluoro- 1,3-benzodioxol-5-yl)ethoxy]-3- pyridyl]-3-(trifluoromethyl)-4,5,6,7- tetrahydroindazol-7- yl]oxy]bicyclo[1.1.1]pentane-1- carboxylic acid |
| 75 (P1) | 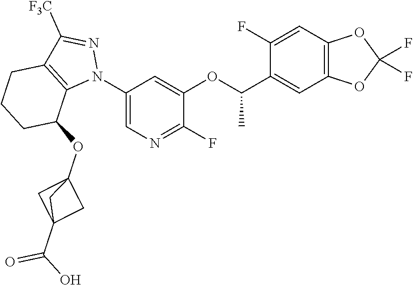 [SMILES: C[C@H](Oc1cc(-n2nc(C(F)(F)F)c3c2[C@@H](OC24CC(C(=O)O)(C2)C4)CCC3)cnc1F)c1cc2c(cc1F)OC(F)(F)O2] | 3-[[(7S)-1-[6-fluoro-5-[(1S)-1-(2,2,6- trifluoro-1,3-benzodioxol-5-yl)ethoxy]- 3-pyridyl]-3-(trifluoromethyl)-4,5,6,7- tetrahydroindazol-7- yl]oxy]bicyclo[1.1.1]pentane-1- carboxylic acid |
| 75 (P2) | 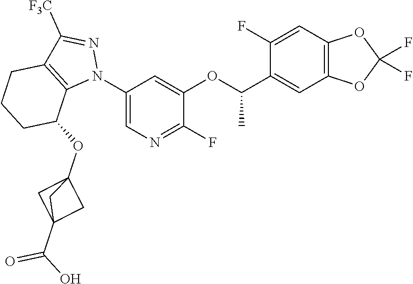 [SMILES: C[C@H](Oc1cc(-n2nc(C(F)(F)F)c3c2[C@H](OC24CC(C(=O)O)(C2)C4)CCC3)cnc1F)c1cc2c(cc1F)OC(F)(F)O2] | 3-[[(7R)-1-[6-fluoro-5-[(1S)-1-(2,2,6- trifluoro-1,3-benzodioxol-5-yl)ethoxy]- 3-pyridyl]-3-(trifluoromethyl)-4,5,6,7- tetrahydroindazol-7- yl]oxy]bicyclo[1.1.1]pentane-1- carboxylic acid |
| 76 (P1) | 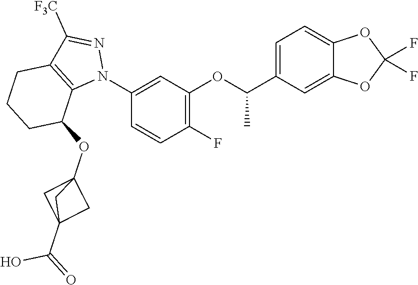 [SMILES: C[C@H](Oc1cc(-n2nc(C(F)(F)F)c3c2[C@@H](OC24CC(C(=O)O)(C2)C4)CCC3)ccc1F)c1ccc2c(c1)OC(F)(F)O2] | 3-[[(7S)-1-[3-[(1S)-1-(2,2-difluoro-1,3- benzodioxol-5-yl)ethoxy]-4-fluoro- phenyl]-3-(trifluoromethyl)-4,5,6,7- tetrahydroindazol-7- yl]oxy]bicyclo[1.1.1]pentane-1- carboxylic acid |
| 76 (P2) | 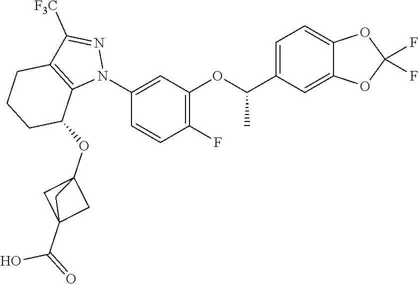 [SMILES: C[C@H](Oc1cc(-n2nc(C(F)(F)F)c3c2[C@H](OC24CC(C(=O)O)(C2)C4)CCC3)ccc1F)c1ccc2c(c1)OC(F)(F)O2] | 3-[[(7R)-1-[3-[(1S)-1-(2,2-difluoro-1,3- benzodioxol-5-yl)ethoxy]-4-fluoro- phenyl]-3-(trifluoromethyl)-4,5,6,7- tetrahydroindazol-7- yl]oxy]bicyclo[1.1.1]pentane-1- carboxylic acid |
| 77 (P1) | 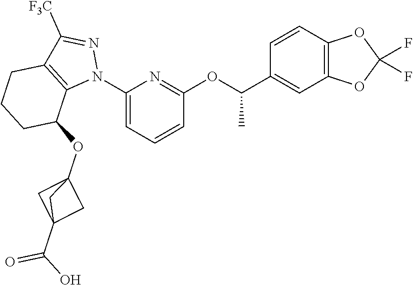 [SMILES: C[C@H](Oc1cccc(-n2nc(C(F)(F)F)c3c2[C@@H](OC24CC(C(=O)O)(C2)C4)CCC3)n1)c1ccc2c(c1)OC(F)(F)O2] | 3-[[(7S)-1-[6-[(1S)-1-(2,2-difluoro-1,3- benzodioxol-5-yl)ethoxy]-2-pyridyl]-3- (trifluoromethyl)-4,5,6,7- tetrahydroindazol-7- yl]oxy]bicyclo[1.1.1]pentane-1- carboxylic acid |
| 77 (P2) | 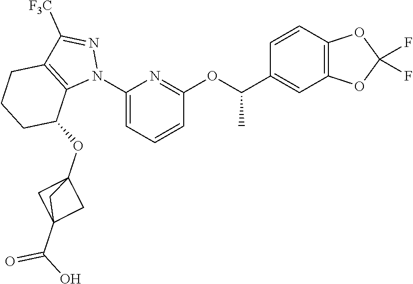 [SMILES: C[C@H](Oc1cccc(-n2nc(C(F)(F)F)c3c2[C@H](OC24CC(C(=O)O)(C2)C4)CCC3)n1)c1ccc2c(c1)OC(F)(F)O2] | 3-[[(7R)-1-[6-[(1S)-1-(2,2-difluoro-1,3- benzodioxol-5-yl)ethoxy]-2-pyridyl]-3- (trifluoromethyl)-4,5,6,7- tetrahydroindazol-7- yl]oxy]bicyclo[1.1.1]pentane-1- carboxylic acid |
| 78 (P1) | 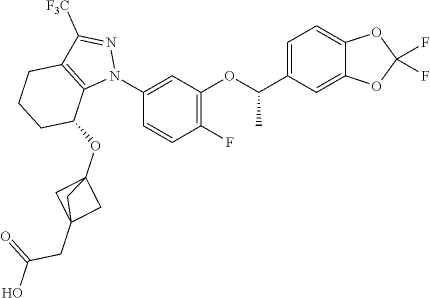 [SMILES: C[C@H](Oc1cc(-n2nc(C(F)(F)F)c3c2[C@H](OC24CC(CC(=O)O)(C2)C4)CCC3)ccc1F)c1ccc2c(c1)OC(F)(F)O2] | 2-[3-[(7S)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]-1- bicyclo[1.1.1]pentanyl]acetic acid |
| 78 (P2) | 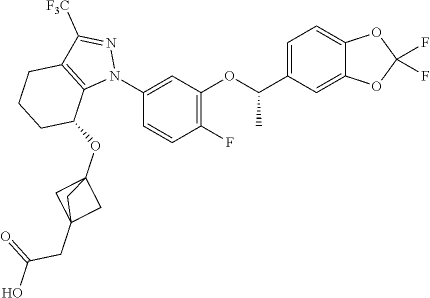 [SMILES: C[C@H](Oc1cc(-n2nc(C(F)(F)F)c3c2[C@H](OC24CC(CC(=O)O)(C2)C4)CCC3)ccc1F)c1ccc2c(c1)OC(F)(F)O2] | 2-[3-[[(7R)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]-1- bicyclo[1.1.1]pentanyl]acetic acid |
| 79 (P1) | 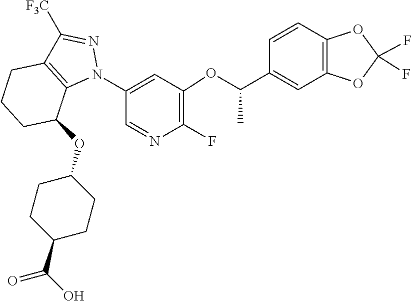 [SMILES: C[C@H](Oc1cc(-n2nc(C(F)(F)F)c3c2[C@@H](O[C@H]2CC[C@H](C(=O)O)CC2)CCC3)cnc1F)c1ccc2c(c1)OC(F)(F)O2] | Trans-4-[[(7S)-1-[5-[(1S)-1-(2,2- difluoro-1,3-benzodioxol-5-yl)ethoxy]- 6-fluoro-3-pyridyl]-3-(trifluoromethyl)- 4,5,6,7-tetrahydroindazol-7- yl]oxy]cyclohexanecarboxylic acid |
| 79 (P2) | 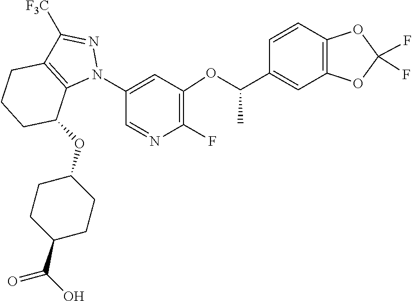 [SMILES: C[C@H](Oc1cc(-n2nc(C(F)(F)F)c3c2[C@H](O[C@H]2CC[C@H](C(=O)O)CC2)CCC3)cnc1F)c1ccc2c(c1)OC(F)(F)O2] | Trans-4-[[(7R)-1-[5-[(1S)-1-(2,2- difluoro-1,3-benzodioxol-5-yl)ethoxy]- 6-fluoro-3-pyridyl]-3-(trifluoromethyl)- 4,5,6,7-tetrahydroindazol-7- yl]oxy]cyclohexanecarboxylic acid |
| 80 (P1) | 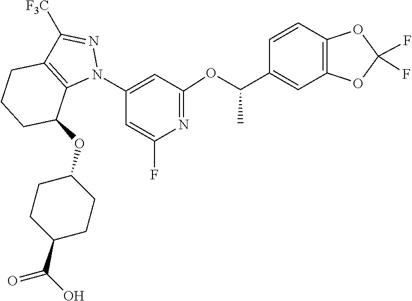 [SMILES: C[C@H](Oc1cc(-n2nc(C(F)(F)F)c3c2[C@@H](O[C@H]2CC[C@H](C(=O)O)CC2)CCC3)cc(F)n1)c1ccc2c(c1)OC(F)(F)O2] | Trans-4-[[(7S)-1-[2-[(1S)-1-(2,2- difluoro-1,3-benzodioxol-5-yl)ethoxy]- 6-fluoro-4-pyridyl]-3-(trifluoromethyl)- 4,5,6,7-tetrahydroindazol-7- yl]oxy]cyclohexanecarboxylic acid |
| 80 (P2) | 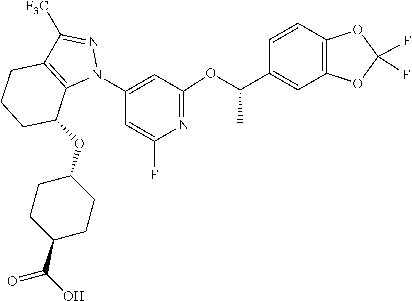 [SMILES: C[C@H](Oc1cc(-n2nc(C(F)(F)F)c3c2[C@H](O[C@H]2CC[C@H](C(=O)O)CC2)CCC3)cc(F)n1)c1ccc2c(c1)OC(F)(F)O2] | Trans-4-[[(7R)-1-[2-[(1S)-1-(2,2- difluoro-1,3-benzodioxol-5-yl)ethoxy]- 6-fluoro-4-pyridyl]-3-(trifluoromethyl)- 4,5,6,7-tetrahydroindazol-7- yl]oxy]cyclohexanecarboxylic acid |
| 81 (P1) | 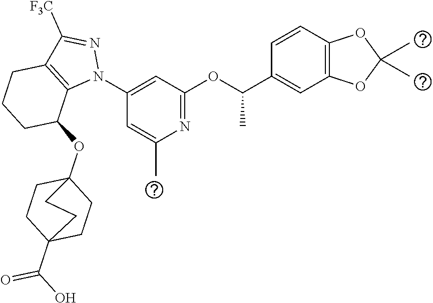 [SMILES: Cc1cc(-n2nc(C(F)(F)F)c3c2[C@@H](OC24CCC(C(=O)O)(CC2)CC4)CCC3)cc(O[C@@H](C)c2ccc3c(c2)OC(C)(C)O3)n1] | 4-[[(7S)-1-[2-[(1S)-1-(2,2-difluoro-1,3- benzodioxol-5-yl)ethoxy]-6-fluoro-4- pyridyl]-3-(trifluoromethyl)-4,5,6,7- tetrahydroindazol-7- yl]oxy]bicyclo[2.2.2]octane-1- carboxylic acid |
| 81 (P2) | 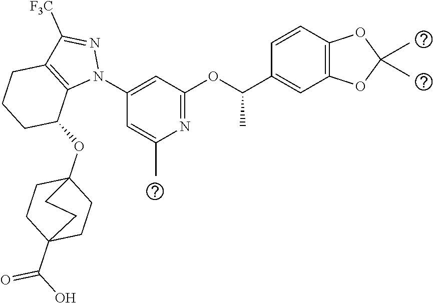 [SMILES: Cc1cc(-n2nc(C(F)(F)F)c3c2[C@H](OC24CCC(C(=O)O)(CC2)CC4)CCC3)cc(O[C@@H](C)c2ccc3c(c2)OC(C)(C)O3)n1] | 4-[[(7R)-1-[2-[(1S)-1-(2,2-difluoro-1,3- benzodioxol-5-yl)ethoxy]-6-fluoro-4- pyridyl]-3-(trifluoromethyl)-4,5,6,7- tetrahydroindazol-7- yl]oxy]bicyclo[2.2.2]octane-1- carboxylic acid |
| 82 (P1) | 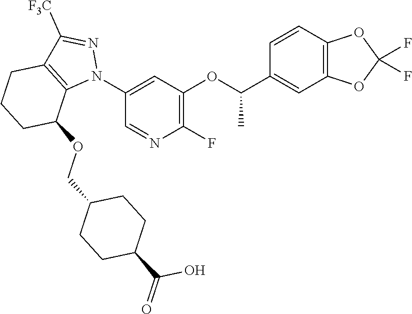 [SMILES: C[C@H](Oc1cc(-n2nc(C(F)(F)F)c3c2[C@@H](OC[C@H]2CC[C@H](C(=O)O)CC2)CCC3)cnc1F)c1ccc2c(c1)OC(F)(F)O2] | Trans-4-[[(7S)-1-[5-[(1S)-1-(2,2- difluoro-1,3-benzodioxol-5-yl)ethoxy]- 6-fluoro-3-pyridyl]-3-(trifluoromethyl)- 4,5,6,7-tetrahydroindazol-7- yl]oxymethyl]cyclohexanecarboxylic acid |
| 82 (P2) | 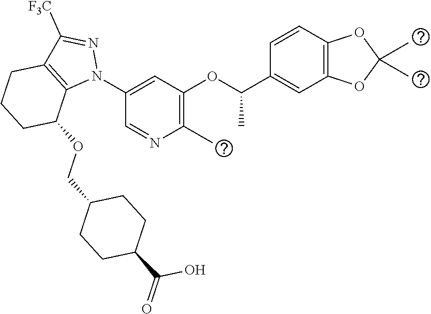 [SMILES: Cc1ncc(-n2nc(C(F)(F)F)c3c2[C@H](OC[C@H]2CC[C@H](C(=O)O)CC2)CCC3)cc1O[C@@H](C)c1ccc2c(c1)OC(C)(C)O2] | Trans-4-[[(7R)-1-[5-[(1S)-1-(2,2- difluoro-1,3-benzodioxol-5-yl)ethoxy]- 6-fluoro-3-pyridyl]-3-(trifluoromethyl)- 4,5,6,7-tetrahydroindazol-7- yl]oxymethyl]cyclohexanecarboxylic acid |
| 83 (P1) | 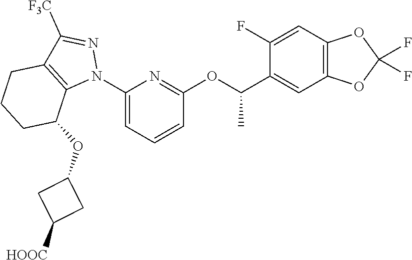 [SMILES: C[C@H](Oc1cccc(-n2nc(C(F)(F)F)c3c2[C@H](O[C@H]2C[C@H](C(=O)O)C2)CCC3)n1)c1cc2c(cc1F)OC(F)(F)O2] | Trans-3-[[(7S)-1-[6-[(1S)-1-(2,2,6- trifluoro-1,3-benzodioxol-5-yl)ethoxy]- 2-pyridyl]-3-(trifluoromethyl)-4,5,6,7- tetrahydroindazol-7- yl]oxy]cyclobutanecarboxylic acid |
| 83 (P2) | 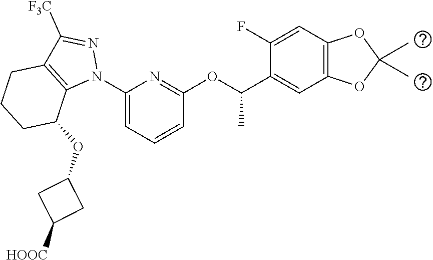 [SMILES: C[C@H](Oc1cccc(-n2nc(C(F)(F)F)c3c2[C@H](O[C@H]2C[C@H](C(=O)O)C2)CCC3)n1)c1cc2c(cc1F)OC(C)(C)O2] | Trans-3-[[(7R)-1-[6-[(1S)-1-(2,2,6- trifluoro-1,3-benzodioxol-5-yl)ethoxy]- 2-pyridyl]-3-(trifluoromethyl)-4,5,6,7- tetrahydroindazol-7- yl]oxy]cyclobutanecarboxylic acid |
| 84 (P1) | 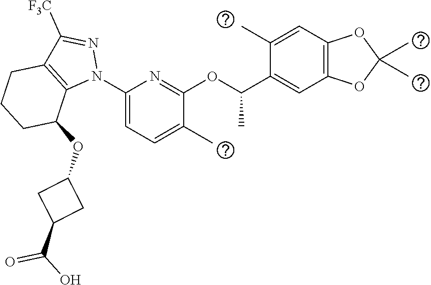 [SMILES: Cc1cc2c(cc1[C@H](C)Oc1nc(-n3nc(C(F)(F)F)c4c3[C@@H](O[C@H]3C[C@H](C(=O)O)C3)CCC4)ccc1C)OC(C)(C)O2] | Trans-3-[(7S)-1-[5-fluoro-6-[(1S)-1- (2,2,6-trifluoro-1,3-benzodioxol-5- yl)ethoxy]-2-pyridyl]-3- (trifluoromethyl)-4,5,6,7- tetrahydroindazol-7- yl]oxy]cyclobutanecarboxylic acid |
| 84 (P2) | 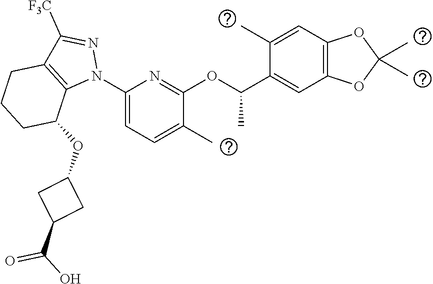 [SMILES: Cc1cc2c(cc1[C@H](C)Oc1nc(-n3nc(C(F)(F)F)c4c3[C@H](O[C@H]3C[C@H](C(=O)O)C3)CCC4)ccc1C)OC(C)(C)O2] | Trans-3-[[(7R)-1-[5-fluoro-6-[(1S)-1- (2,2,6-trifluoro-1,3-benzodioxol-5- yl)ethoxy]-2-pyridyl]-3- (trifluoromethyl)-4,5,6,7- tetrahydroindazol-7- yl]oxy]cyclobutanecarboxylic acid |
| 85 (P1) | 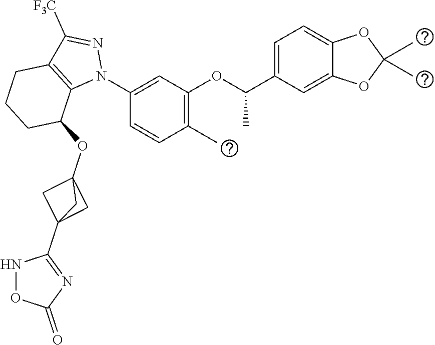 [SMILES: Cc1ccc(-n2nc(C(F)(F)F)c3c2[C@@H](OC24CC(c5nc(=O)o[nH]5)(C2)C4)CCC3)cc1O[C@@H](C)c1ccc2c(c1)OC(C)(C)O2] | 3-[3-[[(7S)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]-1- bicyclo[1.1.1]pentanyl]-2H-1,2,4- oxadiazol-5-one |
| 85 (P2) | 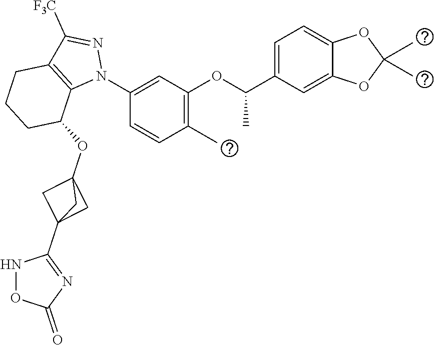 [SMILES: Cc1ccc(-n2nc(C(F)(F)F)c3c2[C@H](OC24CC(c5nc(=O)o[nH]5)(C2)C4)CCC3)cc1O[C@@H](C)c1ccc2c(c1)OC(C)(C)O2] | 3-[3-[[(7R)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]-1- bicyclo[1.1.1]pentanyl]-2H-1,2,4- oxadiazol-5-one |
| 86 (P1) | 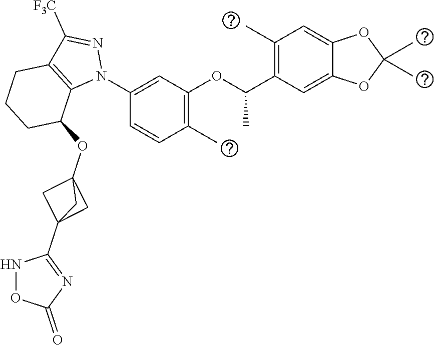 [SMILES: Cc1ccc(-n2nc(C(F)(F)F)c3c2[C@@H](OC24CC(c5nc(=O)o[nH]5)(C2)C4)CCC3)cc1O[C@@H](C)c1cc2c(cc1C)OC(C)(C)O2] | 3-[3-[[(7S)-1-[4-fluoro-3-[(1S)-1-(2,2,6- trifluoro-1,3-benzodioxol-5- yl)ethoxy]phenyl]-3-(trifluoromethyl)- 4,5,6,7-tetrahydroindazol-7-yl]oxy]-1- bicyclo[1.1.1]pentanyl]-2H-1,2,4- oxadiazol-5-one |
| 86 (P2) | 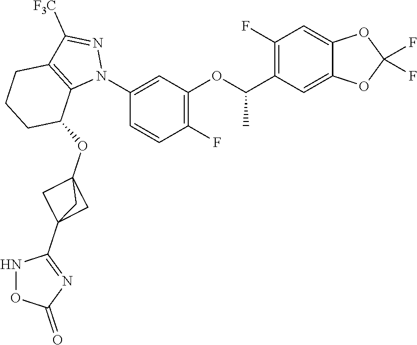 [SMILES: C[C@H](Oc1cc(-n2nc(C(F)(F)F)c3c2[C@H](OC24CC(c5nc(=O)o[nH]5)(C2)C4)CCC3)ccc1F)c1cc2c(cc1F)OC(F)(F)O2] | 3-[3-[[(7R)-1-[4-fluoro-3-[(1S)-1-(2,2,6- trifluoro-1,3-benzodioxol-5- yl)ethoxy]phenyl]-3-(trifluoromethyl)- 4,5,6,7-tetrahydroindazol-7-yl]oxy]-1- bicyclo[1.1.1]pentanyl]-2H-1,2,4- oxadiazol-5-one |
| 87 | 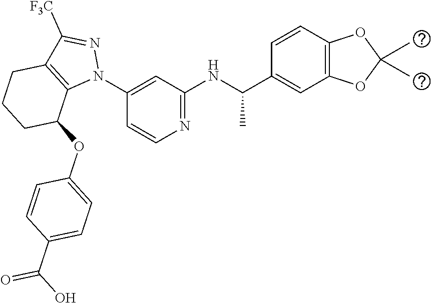 [SMILES: C[C@H](Nc1cc(-n2nc(C(F)(F)F)c3c2[C@@H](Oc2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)ethyl]amino]-4- pyridyl]-3-(trifluoromethyl)-4,5,6,7- tetrahydroindazol-7-yl]oxy]benzoic acid |
| 88 | 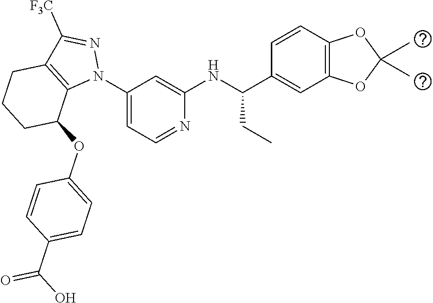 [SMILES: CC[C@H](Nc1cc(-n2nc(C(F)(F)F)c3c2[C@@H](Oc2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)propyl]amino]-4- pyridyl]-3-(trifluoromethyl)-4,5,6,7- tetrahydroindazol-7-yl]oxy]benzoic acid |
| 89 | 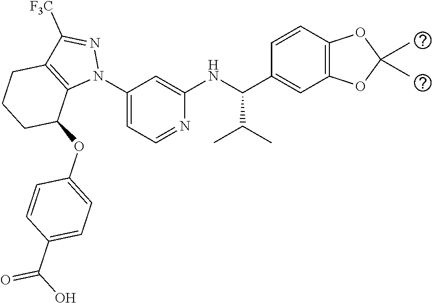 [SMILES: CC(C)[C@H](Nc1cc(-n2nc(C(F)(F)F)c3c2[C@@H](Oc2ccc(C(=O)O)cc2)CCC3)ccn1)c1ccc2c(c1)OC(C)(C)O2] | 4-[[(7S)-1-[2-[[(1S)-1-(2,2-difluoro-1,3- benzodioxol-5-yl)-2-methyl- propyl]amino]-4-pyridyl]-3- (trifluoromethyl)-4,5,6,7- tetrahydroindazol-7-yl]oxy]benzoic acid |
| 90 | 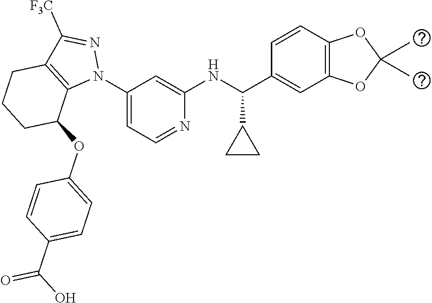 [SMILES: CC1(C)Oc2ccc([C@@H](Nc3cc(-n4nc(C(F)(F)F)c5c4[C@@H](Oc4ccc(C(=O)O)cc4)CCC5)ccn3)C3CC3)cc2O1] | 4-[[(7S)-1-[2-[[(S)-cyclopropyl-(2,2- difluoro-1,3-benzodioxol-5- yl)methyl]amino]-4-pyridyl]-3- (trifluoromethyl)-4,5,6,7- tetrahydroindazol-7-yl]oxy]benzoic acid |
| 91 | 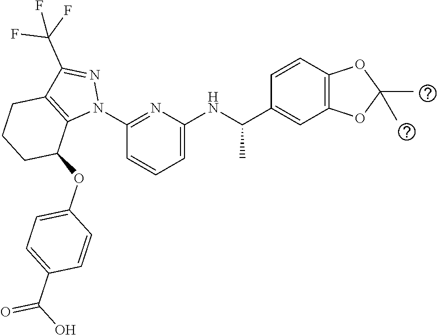 [SMILES: C[C@H](Nc1cccc(-n2nc(C(F)(F)F)c3c2[C@@H](Oc2ccc(C(=O)O)cc2)CCC3)n1)c1ccc2c(c1)OC(C)(C)O2] | 4-[[(7S)-1-[6-[(1S)-1-(2,2-difluoro-1,3- benzodioxol-5-yl)ethyl]amino]-2- pyridyl]-3-(trifluoromethyl)-4,5,6,7- tetrahydroindazol-7-yl]oxy]benzoic acid |
| 92 | 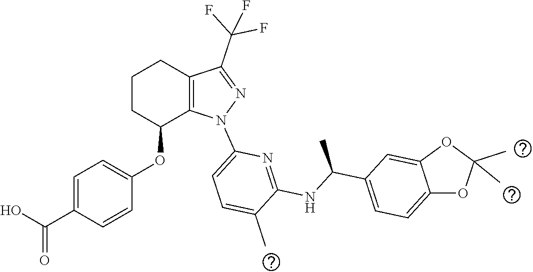 [SMILES: Cc1ccc(-n2nc(C(F)(F)F)c3c2[C@@H](Oc2ccc(C(=O)O)cc2)CCC3)nc1N[C@@H](C)c1ccc2c(c1)OC(C)(C)O2] | 4-[[(7S)-1-[6-[[(1S)-1-(2,2-difluoro-1,3- benzodioxol-5-yl)ethyl]amino]-5-fluoro- 2-pyridyl]-3-(trifluoromethyl)-4,5,6,7- tetrahydroindazol-7-yl]oxy]benzoic acid |
| 93 | 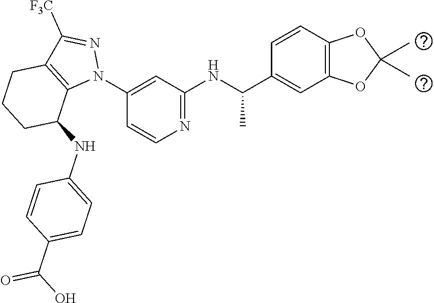 [SMILES: C[C@H](Nc1cc(-n2nc(C(F)(F)F)c3c2[C@@H](Nc2ccc(C(=O)O)cc2)CCC3)ccn1)c1ccc2c(c1)OC(C)(C)O2] | 4-[[(7S)-1-[2-[[(1S)-1-(2,2-difluoro-1,3- benzodioxol-5-yl)ethyl]amino]-4- pyridyl]-3-(trifluoromethyl)-4,5,6,7- tetrahydroindazol-7-yl]amino]benzoic acid |
| 94 (P1) | 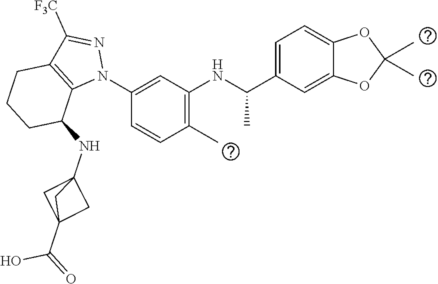 [SMILES: Cc1ccc(-n2nc(C(F)(F)F)c3c2[C@@H](NC24CC(C(=O)O)(C2)C4)CCC3)cc1N[C@@H](C)c1ccc2c(c1)OC(C)(C)O2] | 3-[[(7S)-1-[3-[[(1S)-1-(2,2-difluoro-1,3- benzodioxol-5-yl)ethyl]amino]-4-fluoro- phenyl]-3-(trifluoromethyl)-4,5,6,7- tetrahydroindazol-7- yl]amino]bicyclo[1.1.1]pentane-1- carboxylic acid |
| 94 (P2) | 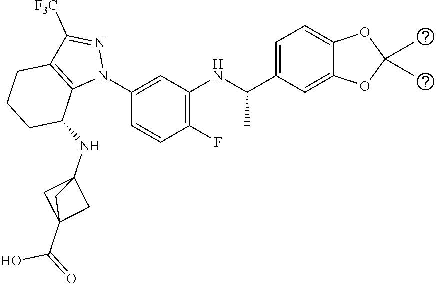 [SMILES: C[C@H](Nc1cc(-n2nc(C(F)(F)F)c3c2[C@H](NC24CC(C(=O)O)(C2)C4)CCC3)ccc1F)c1ccc2c(c1)OC(C)(C)O2] | 3-[[(7R)-1-[3-[[(1S)-1-(2,2-difluoro-1,3- benzodioxol-5-yl)ethyl]amino]-4-fluoro- phenyl]-3-(trifluoromethyl)-4,5,6,7- tetrahydroindazol-7- yl]amino]bicyclo[1.1.1]pentane-1- carboxylic acid |
| 95 (P1) | 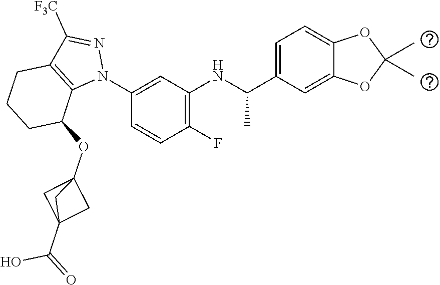 [SMILES: C[C@H](Nc1cc(-n2nc(C(F)(F)F)c3c2[C@@H](OC24CC(C(=O)O)(C2)C4)CCC3)ccc1F)c1ccc2c(c1)OC(C)(C)O2] | 3-[[(7S)-1-[3-[(1S)-1-(2,2-difluoro-1,3- benzodioxol-5-yl)ethyl]amino]-4-fluoro- phenyl]-3-(trifluoromethyl)-4,5,6,7- tetrahydroindazol-7- yl]oxy]bicyclo[1.1.1]pentane-1- carboxylic acid |
| 95 (P2) | 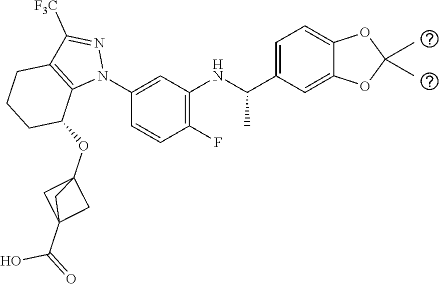 [SMILES: C[C@H](Nc1cc(-n2nc(C(F)(F)F)c3c2[C@H](OC24CC(C(=O)O)(C2)C4)CCC3)ccc1F)c1ccc2c(c1)OC(C)(C)O2] | 3-[[(7R)-1-[3-[[(1S)-1-(2,2-difluoro-1,3- benzodioxol-5-yl)ethyl]amino]-4-fluoro- phenyl]-3-(trifluoromethyl)-4,5,6,7- tetrahydroindazol-7- yl]oxy]bicyclo[1.1.1]pentane-1- carboxylic acid |
| 96 (P1) | 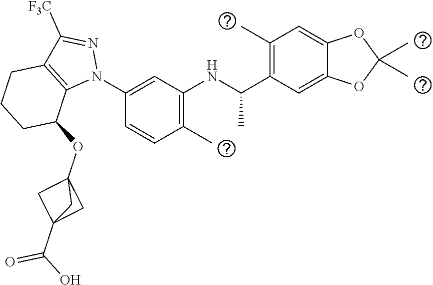 [SMILES: Cc1ccc(-n2nc(C(F)(F)F)c3c2[C@@H](OC24CC(C(=O)O)(C2)C4)CCC3)cc1N[C@@H](C)c1cc2c(cc1C)OC(C)(C)O2] | 3-[[(7S)-1-[4-fluoro-3-[[(1S)-1-(2,2,6- trifluoro-1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 96 (P2) | 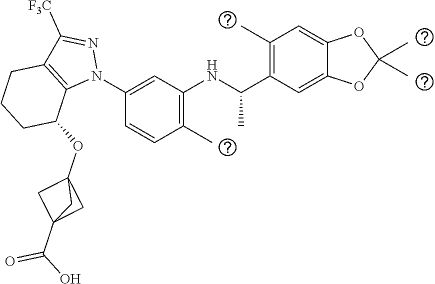 [SMILES: Cc1ccc(-n2nc(C(F)(F)F)c3c2[C@H](OC24CC(C(=O)O)(C2)C4)CCC3)cc1N[C@@H](C)c1cc2c(cc1C)OC(C)(C)O2] | 3-[[(7R)-1-[4-fluoro-3-[[(1S)-1-(2,2,6- trifluoro-1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 97 (P1) | 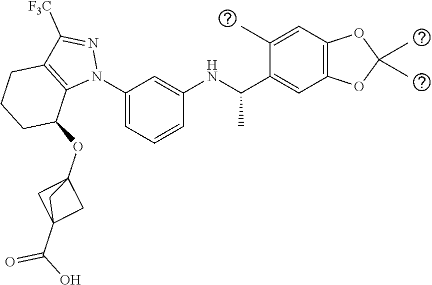 [SMILES: Cc1cc2c(cc1[C@H](C)Nc1cccc(-n3nc(C(F)(F)F)c4c3[C@@H](OC35CC(C(=O)O)(C3)C5)CCC4)c1)OC(C)(C)O2] | 3-[[(7S)-1-[3-[[(1S)-1-(2,2,6-trifluoro- 1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 97 (P2) | 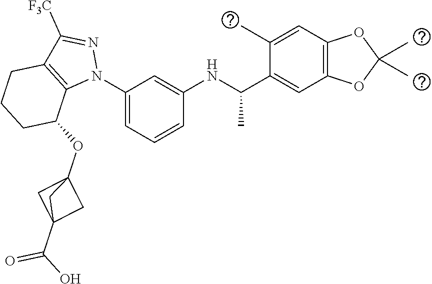 [SMILES: Cc1cc2c(cc1[C@H](C)Nc1cccc(-n3nc(C(F)(F)F)c4c3[C@H](OC35CC(C(=O)O)(C3)C5)CCC4)c1)OC(C)(C)O2] | 3-[[(7R)-1-[3-[[(1S)-1-(2,2,6-trifluoro- 1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 98 (P1) | 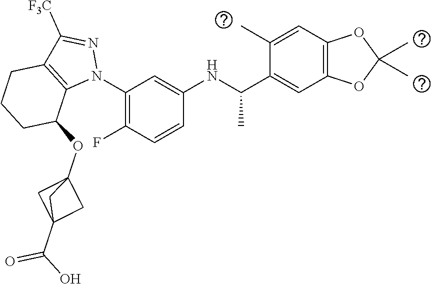 [SMILES: Cc1cc2c(cc1[C@H](C)Nc1ccc(F)c(-n3nc(C(F)(F)F)c4c3[C@@H](OC35CC(C(=O)O)(C3)C5)CCC4)c1)OC(C)(C)O2] | 3-[[(7S)-1-[2-fluoro-5-[[(1S)-1-(2,2,6- trifluoro-1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 98 (P2) | 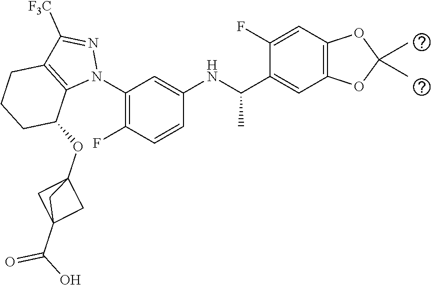 [SMILES: C[C@H](Nc1ccc(F)c(-n2nc(C(F)(F)F)c3c2[C@H](OC24CC(C(=O)O)(C2)C4)CCC3)c1)c1cc2c(cc1F)OC(C)(C)O2] | 3-[[(7R)-1-[2-fluoro-5-[(1S)-1-(2,2,6- trifluoro-1,3-benzodioxol-5- yl)ethyl]amino]phenyl]-3- (trifluoromethyl)-4,5,6,7- tetrahydroindazol-7- yl]oxy]bicyclo[1.1.1]pentane-1- carboxylic acid |
| 99 (P1) | 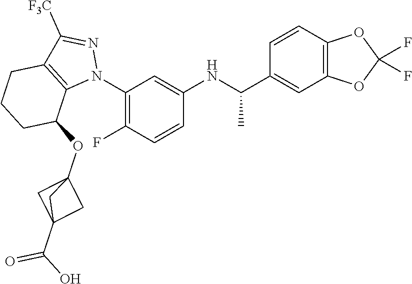 [SMILES: C[C@H](Nc1ccc(F)c(-n2nc(C(F)(F)F)c3c2[C@@H](OC24CC(C(=O)O)(C2)C4)CCC3)c1)c1ccc2c(c1)OC(F)(F)O2] | 3-[[(7S)-1-[5-[(1S)-1-(2,2-difluoro-1,3- benzodioxol-5-yl)ethyl]amino]-2-fluoro- phenyl]-3-(trifluoromethyl)-4,5,6,7- tetrahydroindazol-7- yl]oxy]bicyclo[1.1.1]pentane-1- carboxylic acid |
| 99 (P2) | 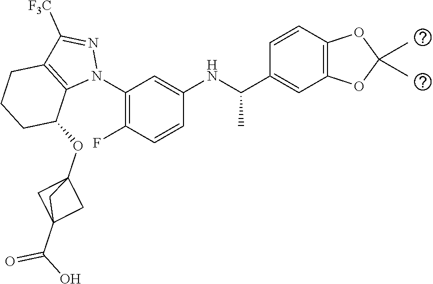 [SMILES: C[C@H](Nc1ccc(F)c(-n2nc(C(F)(F)F)c3c2[C@H](OC24CC(C(=O)O)(C2)C4)CCC3)c1)c1ccc2c(c1)OC(C)(C)O2] | 3-[[(7R)-1-[5-[(1S)-1-(2,2-difluoro-1,3- benzodioxol-5-yl)ethyl]amino]-2-fluoro- phenyl]-3-(trifluoromethyl)-4,5,6,7- tetrahydroindazol-7- yl]oxy]bicyclo[1.1.1]pentane-1- carboxylic acid |
| 100 (P1) | 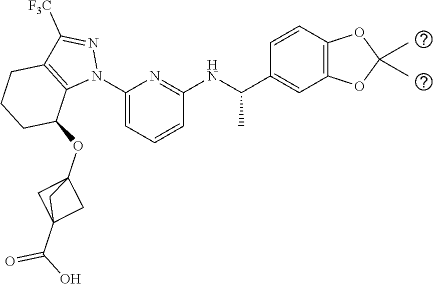 [SMILES: C[C@H](Nc1cccc(-n2nc(C(F)(F)F)c3c2[C@@H](OC24CC(C(=O)O)(C2)C4)CCC3)n1)c1ccc2c(c1)OC(C)(C)O2] | 3-[[(7S)-1-[6-[[(1S)-1-(2,2-difluoro-1,3- benzodioxol-5-yl)ethyl]amino]-2- pyridyl]-3-(trifluoromethyl)-4,5,6,7- tetrahydroindazol-7- yl]oxy]bicyclo[1.1.1]pentane-1- carboxylic acid |
| 100 (P2) | 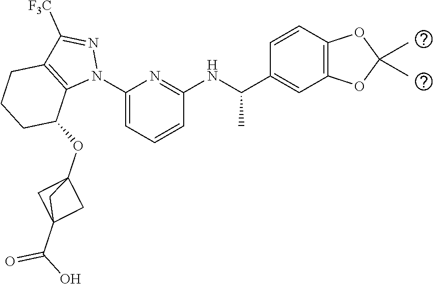 [SMILES: C[C@H](Nc1cccc(-n2nc(C(F)(F)F)c3c2[C@H](OC24CC(C(=O)O)(C2)C4)CCC3)n1)c1ccc2c(c1)OC(C)(C)O2] | 3-[[(7R)-1-[6-[[(1S)-1-(2,2-difluoro-1,3- benzodioxol-5-yl)ethyl]amino]-2- pyridyl]-3-(trifluoromethyl)-4,5,6,7- tetrahydroindazol-7- yl]oxy]bicyclo[1.1.1]pentane-1- carboxylic acid |
| 101 (P1) | 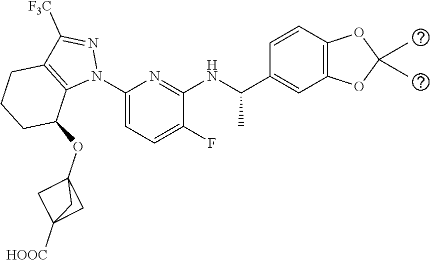 [SMILES: C[C@H](Nc1nc(-n2nc(C(F)(F)F)c3c2[C@@H](OC24CC(C(=O)O)(C2)C4)CCC3)ccc1F)c1ccc2c(c1)OC(C)(C)O2] | 3-[[(7S)-1-[6-[[(1S)-1-(2,2-difluoro-1,3- benzodioxol-5-yl)ethyl]amino]-5-fluoro- 2-pyridyl]-3-(trifluoromethyl)-4,5,6,7- tetrahydroindazol-7- yl]oxy]bicyclo[1.1.1]pentane-1- carboxylic acid |
| 101 (P2) | 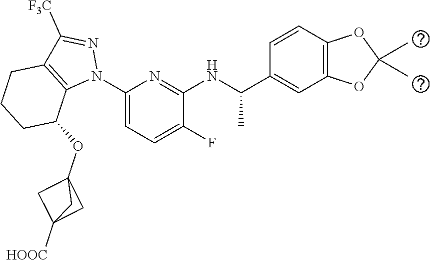 [SMILES: C[C@H](Nc1nc(-n2nc(C(F)(F)F)c3c2[C@H](OC24CC(C(=O)O)(C2)C4)CCC3)ccc1F)c1ccc2c(c1)OC(C)(C)O2] | 3-[[(7R)-1-[6-[[(1S)-1-(2,2-difluoro-1,3- benzodioxol-5-yl)ethyl]amino]-5-fluoro- 2-pyridyl]-3-(trifluoromethyl)-4,5,6,7- tetrahydroindazol-7- yl]oxy]bicyclo[1.1.1]pentane-1- carboxylic acid |
| 102 (P1) | 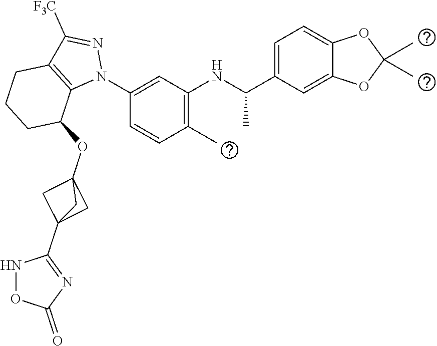 [SMILES: Cc1ccc(-n2nc(C(F)(F)F)c3c2[C@@H](OC24CC(c5nc(=O)o[nH]5)(C2)C4)CCC3)cc1N[C@@H](C)c1ccc2c(c1)OC(C)(C)O2] | 3-[3-[[(7S)-1-[3-[(1S)-1-(2,2-difluoro- 1,3-benzodioxol-5-yl)ethyl]amino]-4- fluoro-phenyl]-3-(trifluoromethyl)- 4,5,6,7-tetrahydroindazol-7-yl]oxy]-1- bicyclo[1.1.1]pentanyl]-2H-1,2,4- oxadiazol-5-one |
| 102 (P2) | 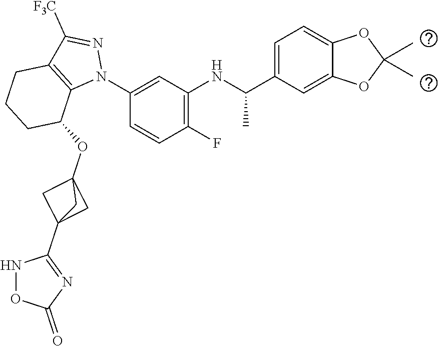 [SMILES: C[C@H](Nc1cc(-n2nc(C(F)(F)F)c3c2[C@H](OC24CC(c5nc(=O)o[nH]5)(C2)C4)CCC3)ccc1F)c1ccc2c(c1)OC(C)(C)O2] | 3-[3-[[(7R)-1-[3-[[(1S)-1-(2,2-difluoro- 1,3-benzodioxol-5-yl)ethyl]amino]-4- fluoro-phenyl]-3-(trifluoromethyl)- 4,5,6,7-tetrahydroindazol-7-yl]oxy]-1- bicyclo[1.1.1]pentanyl]-2H-1,2,4- oxadiazol-5-one |
| 103 (P1) | 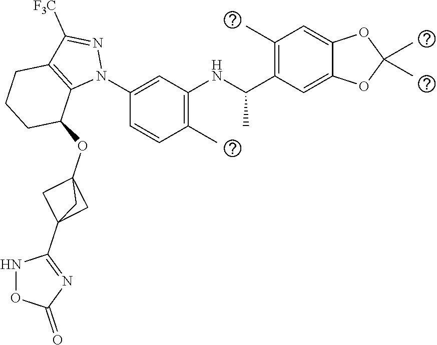 [SMILES: Cc1ccc(-n2nc(C(F)(F)F)c3c2[C@@H](OC24CC(c5nc(=O)o[nH]5)(C2)C4)CCC3)cc1N[C@@H](C)c1cc2c(cc1C)OC(C)(C)O2] | 3-[3-[[(7S)-1-[4-fluoro-3-[[(1S)-1- (2,2,6-trifluoro-1,3-benzodioxol-5- yl)ethyl]amino]phenyl]-3- (trifluoromethyl)-4,5,6,7- tetrahydroindazol-7-yl]oxy]-1- bicyclo[1.1.1]pentanyl]-2H-1,2,4- oxadiazol-5-one |
| 103 (P2) | 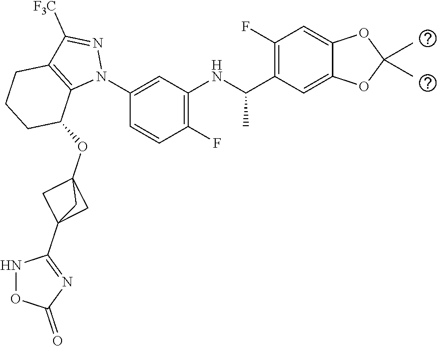 [SMILES: C[C@H](Nc1cc(-n2nc(C(F)(F)F)c3c2[C@H](OC24CC(c5nc(=O)o[nH]5)(C2)C4)CCC3)ccc1F)c1cc2c(cc1F)OC(C)(C)O2] | 3-[3-[[(7R)-1-[4-fluoro-3-[[(1S)-1- (2,2,6-trifluoro-1,3-benzodioxol-5- yl)ethyl]amino]phenyl]-3- (trifluoromethyl)-4,5,6,7- tetrahydroindazol-7-yl]oxy]-1- bicyclo[1.1.1]pentanyl]-2H-1,2,4- oxadiazol-5-one |
| 104 | 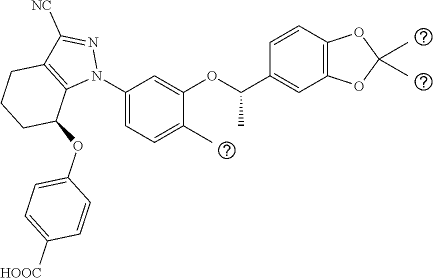 [SMILES: Cc1ccc(-n2nc(C#N)c3c2[C@@H](Oc2ccc(C(=O)O)cc2)CCC3)cc1O[C@@H](C)c1ccc2c(c1)OC(C)(C)O2] | 4-[[(7S)-3-cyano-1-[3-[(1S)-1-(2,2- difluoro-1,3-benzodioxol-5-yl)ethoxy]- 4-fluoro-phenyl]-4,5,6,7- tetrahydroindazol-7-yl]oxy]benzoic acid |
| 105 (P1) | 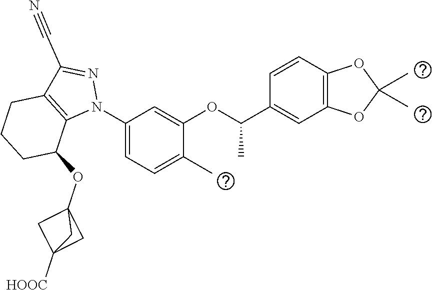 [SMILES: Cc1ccc(-n2nc(C#N)c3c2[C@@H](OC24CC(C(=O)O)(C2)C4)CCC3)cc1O[C@@H](C)c1ccc2c(c1)OC(C)(C)O2] | 3-[[(7S)-3-cyano-1-[3-[(1S)-1-(2,2- difluoro-1,3-benzodioxol-5-yl)ethoxy]- 4-fluoro-phenyl]-4,5,6,7- tetrahydroindazol-7- yl]oxy]bicyclo[1.1.1]pentane-1- carboxylic acid |
| 105 (P2) | 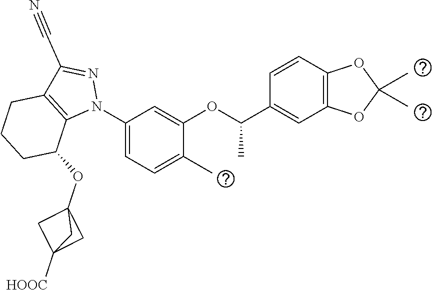 [SMILES: Cc1ccc(-n2nc(C#N)c3c2[C@H](OC24CC(C(=O)O)(C2)C4)CCC3)cc1O[C@@H](C)c1ccc2c(c1)OC(C)(C)O2] | 3-[[(7R)-3-cyano-1-[3-[(1S)-1-(2,2- difluoro-1,3-benzodioxol-5-yl)ethoxy]- 4-fluoro-phenyl]-4,5,6,7- tetrahydroindazol-7- yl]oxy]bicyclo[1.1.1]pentane-1- carboxylic acid |
| 106 (P1) | 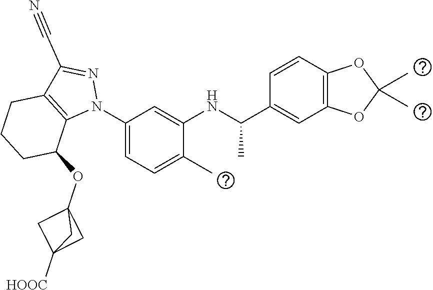 [SMILES: Cc1ccc(-n2nc(C#N)c3c2[C@@H](OC24CC(C(=O)O)(C2)C4)CCC3)cc1N[C@@H](C)c1ccc2c(c1)OC(C)(C)O2] | 3-[[(7S)-3-cyano-1-[3-[(1S)-1-(2,2- difluoro-1,3-benzodioxol-5- yl)ethyl]amino]-4-fluoro-phenyl]- 4,5,6,7-tetrahydroindazol-7- yl]oxy]bicyclo[1.1.1]pentane-1- carboxylic acid |
| 106 (P2) | 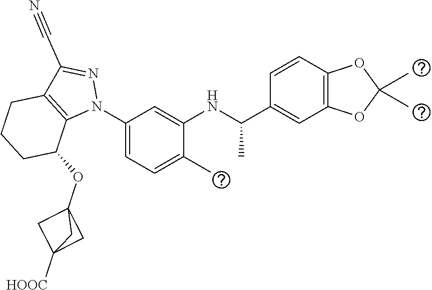 [SMILES: Cc1ccc(-n2nc(C#N)c3c2[C@H](OC24CC(C(=O)O)(C2)C4)CCC3)cc1N[C@@H](C)c1ccc2c(c1)OC(C)(C)O2] | 3-[[(7R)-3-cyano-1-[3-[(1S)-1-(2,2- difluoro-1,3-benzodioxol-5- yl)ethyl]amino]-4-fluoro-phenyl]- 4,5,6,7-tetrahydroindazol-7- yl]oxy]bicyclo[1.1.1]pentane-1- carboxylic acid |
| 107 (P1) | 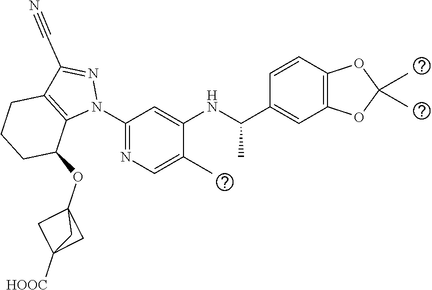 [SMILES: Cc1cnc(-n2nc(C#N)c3c2[C@@H](OC24CC(C(=O)O)(C2)C4)CCC3)cc1N[C@@H](C)c1ccc2c(c1)OC(C)(C)O2] | 3-[[(7S)-3-cyano-1-[4-[(1S)-1-(2,2- difluoro-1,3-benzodioxol-5- yl)ethyl]amino]-5-fluoro-2-pyridyl]- 4,5,6,7-tetrahydroindazol-7- yl]oxy]bicyclo[1.1.1]pentane-1- carboxylic acid |
| 107 (P2) | 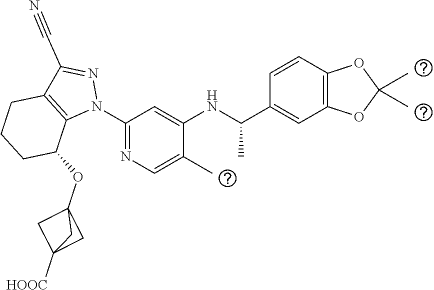 [SMILES: Cc1cnc(-n2nc(C#N)c3c2[C@H](OC24CC(C(=O)O)(C2)C4)CCC3)cc1N[C@@H](C)c1ccc2c(c1)OC(C)(C)O2] | 3-[[(7R)-3-cyano-1-[4-[[(1S)-1-(2,2- difluoro-1,3-benzodioxol-5- yl)ethyl]amino]-5-fluoro-2-pyridyl]- 4,5,6,7-tetrahydroindazol-7- yl]oxy]bicyclo[1.1.1]pentane-1- carboxylic acid |
| 108 (P1) | 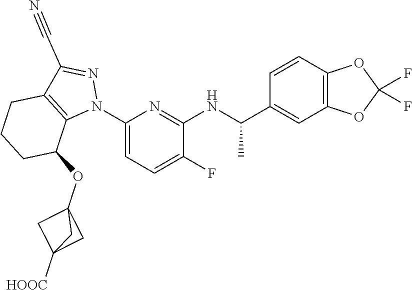 [SMILES: C[C@H](Nc1nc(-n2nc(C#N)c3c2[C@@H](OC24CC(C(=O)O)(C2)C4)CCC3)ccc1F)c1ccc2c(c1)OC(F)(F)O2] | 3-[[(7S)-3-cyano-1-[6-[(1S)-1-(2,2- difluoro-1,3-benzodioxol-5- yl)ethyl]amino]-5-fluoro-2-pyridyl]- 4,5,6,7-tetrahydroindazol-7- yl]oxy]bicyclo[1.1.1]pentane-1- carboxylic acid |
| 108 (P2) | 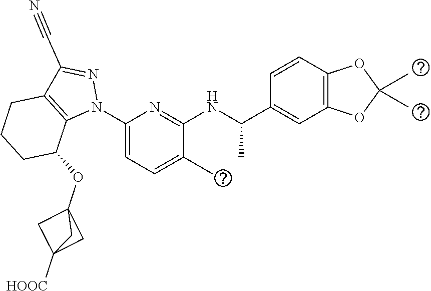 [SMILES: Cc1ccc(-n2nc(C#N)c3c2[C@H](OC24CC(C(=O)O)(C2)C4)CCC3)nc1N[C@@H](C)c1ccc2c(c1)OC(C)(C)O2] | 3-[[(7R)-3-cyano-1-[6-[[(1S)-1-(2,2- difluoro-1,3-benzodioxol-5- yl)ethyl]amino]-5-fluoro-2-pyridyl]- 4,5,6,7-tetrahydroindazol-7- yl]oxy]bicyclo[1.1.1]pentane-1- carboxylic acid |
| 109 | 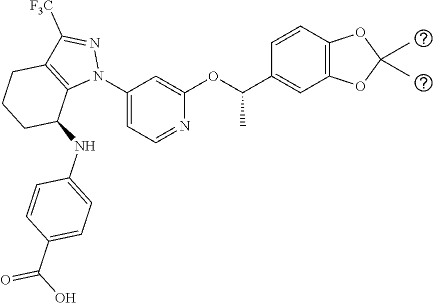 [SMILES: C[C@H](Oc1cc(-n2nc(C(F)(F)F)c3c2[C@@H](Nc2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7-yl]amino]benzoic acid |
| 110 | 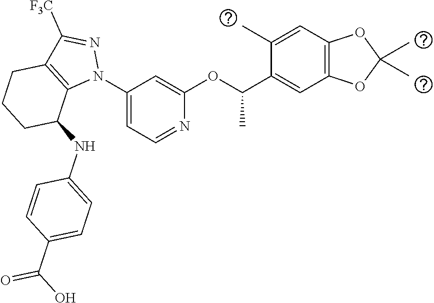 [SMILES: Cc1cc2c(cc1[C@H](C)Oc1cc(-n3nc(C(F)(F)F)c4c3[C@@H](Nc3ccc(C(=O)O)cc3)CCC4)ccn1)OC(C)(C)O2] | 4-[[(7S)-1-[2-[(1S)-1-(2,2,6-trifluoro- 1,3-benzodioxol-5-yl)ethoxy]-4- pyridyl]-3-(trifluoromethyl)-4,5,6,7- tetrahydroindazol-7-yl]amino]benzoic acid |
| 111 | 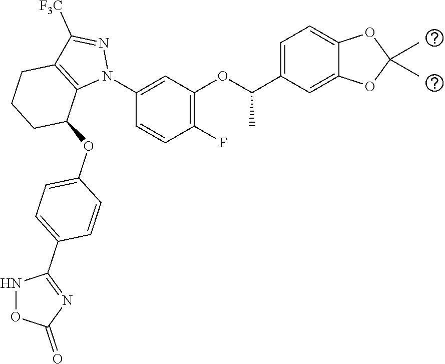 [SMILES: C[C@H](Oc1cc(-n2nc(C(F)(F)F)c3c2[C@@H](Oc2ccc(-c4nc(=O)o[nH]4)cc2)CCC3)ccc1F)c1ccc2c(c1)OC(C)(C)O2] | 3-[4-[[(7S)-1-[3-[(1S)-1-(2,2-difluoro- 1,3-benzodioxol-5-yl)ethoxy]-4-fluoro- phenyl]-3-(trifluoromethyl)-4,5,6,7- tetrahydroindazol-7-yl]oxy]phenyl]-2H- 1,2,4-oxadiazol-5-one |
| 112 | 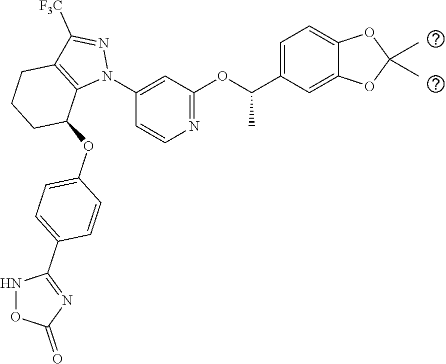 [SMILES: C[C@H](Oc1cc(-n2nc(C(F)(F)F)c3c2[C@@H](Oc2ccc(-c4nc(=O)o[nH]4)cc2)CCC3)ccn1)c1ccc2c(c1)OC(C)(C)O2] | 3-[4-[[(7S)-1-[2-[(1S)-1-(2,2-difluoro- 1,3-benzodioxol-5-yl)ethoxy]-4- pyridyl]-3-(trifluoromethyl)-4,5,6,7- tetrahydroindazol-7-yl]oxy]phenyl]-2H- 1,2,4-oxadiazol-5-one |
| 113 | 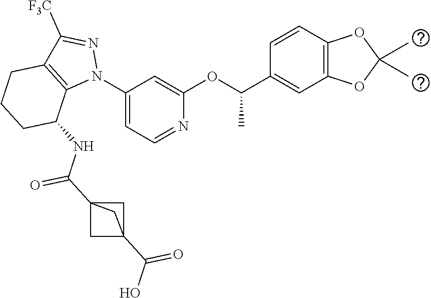 [SMILES: C[C@H](Oc1cc(-n2nc(C(F)(F)F)c3c2[C@H](NC(=O)C24CC(C(=O)O)(C2)C4)CCC3)ccn1)c1ccc2c(c1)OC(C)(C)O2] | 3-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]carbamoyl]bicyclo[1.1.1]pentane-1- carboxylic acid |
| 114 | 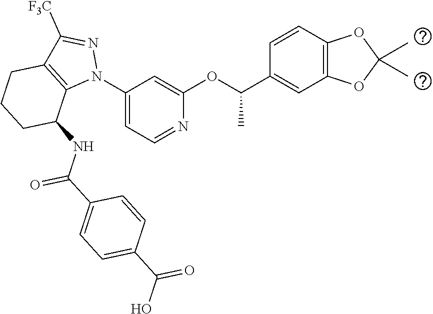 [SMILES: C[C@H](Oc1cc(-n2nc(C(F)(F)F)c3c2[C@@H](NC(=O)c2ccc(C(=O)O)cc2)CCC3)ccn1)c1ccc2c(c1)OC(C)(C)O2] | 4-[[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]carbamoyl]benzoic acid |
| 115 (P1) | 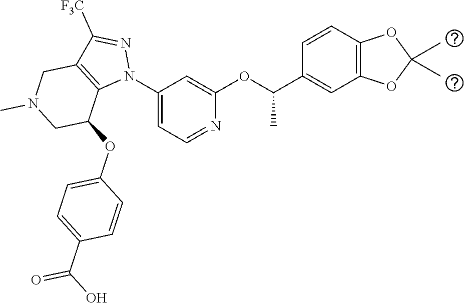 | 4-[[(7S)-1-[2-[(1S)-1-(2,2-difluoro-1,3- benzodioxol-5-yl)ethoxy]-4-pyridyl]-5- methyl-3-(trifluoromethyl)-6,7-dihydro- 4H-pyrazolo[4,3-c]pyridin-7- yl]oxy]benzoic acid |
| 115 (P2) | 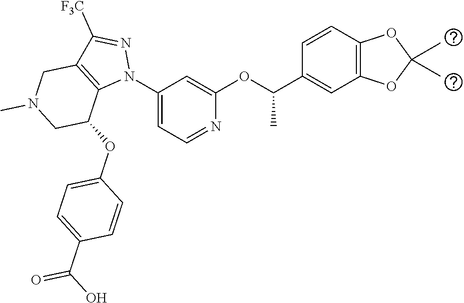 | 4-[[(7R)-1-[2-[(1S)-1-(2,2-difluoro-1,3- benzodioxol-5-yl)ethoxy]-4-pyridyl]-5- methyl-3-(trifluoromethyl)-6,7-dihydro- 4H-pyrazolo[4,3-c]pyridin-7- yl]oxy]benzoic acid |
| 116 | 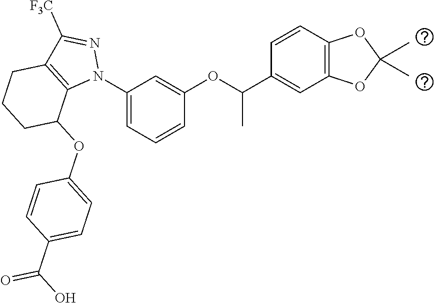 [SMILES: CC(Oc1cccc(-n2nc(C(F)(F)F)c3c2C(Oc2ccc(C(=O)O)cc2)CCC3)c1)c1ccc2c(c1)OC(C)(C)O2] | 4-[[1-[3-[1-(2,2-difluoro-1,3- benzodioxol-5-yl)ethylsulfanyl]phenyl]- 3-(trifluoromethyl)-4,5,6,7- tetrahydroindazol-7-yl]oxy]benzoic acid |
| 117 | 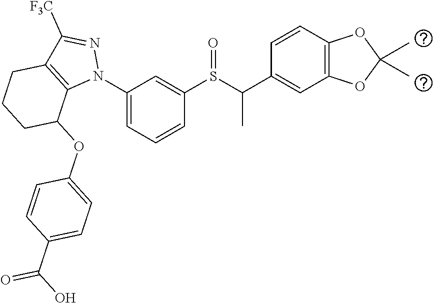 [SMILES: CC(c1ccc2c(c1)OC(C)(C)O2)S(=O)c1cccc(-n2nc(C(F)(F)F)c3c2C(Oc2ccc(C(=O)O)cc2)CCC3)c1] | 4-[[1-[3-[1-(2,2-difluoro-1,3- benzodioxol-5-yl)ethylsulfinyl]phenyl]- 3-(trifluoromethyl)-4,5,6,7- tetrahydroindazol-7-yl]oxy]benzoic acid |
| 118 | 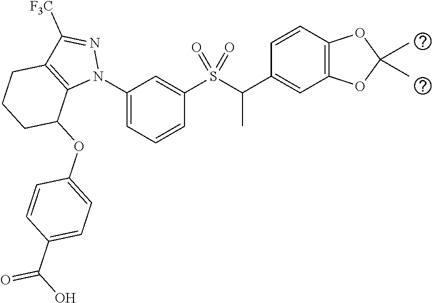 [SMILES: CC(c1ccc2c(c1)OC(C)(C)O2)S(=O)(=O)c1cccc(-n2nc(C(F)(F)F)c3c2C(Oc2ccc(C(=O)O)cc2)CCC3)c1] | 4-[[1-[3-[1-(2,2-difluoro-1,3- benzodioxol-5-yl)ethylsulfonyl]phenyl]- 3-(trifluoromethyl)-4,5,6,7- tetrahydroindazol-7-yl]oxy]benzoic acid |
| 119 | 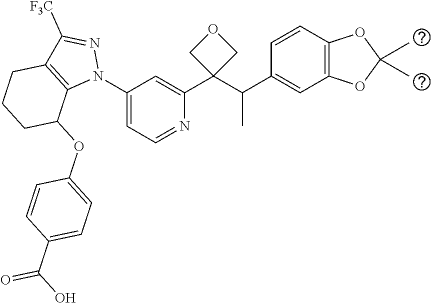 [SMILES: CC(c1ccc2c(c1)OC(C)(C)O2)C1(c2cc(-n3nc(C(F)(F)F)c4c3C(Oc3ccc(C(=O)O)cc3)CCC4)ccn2)COC1] | 4-[[1-[2-[3-[1-(2,2-difluoro-1,3- benzodioxol-5-yl)ethyl]oxetan-3-yl]-4- pyridyl]-3-(trifluoromethyl)-4,5,6,7- tetrahydroindazol-7-yl]oxy]benzoic acid |
| 120 (P1) | 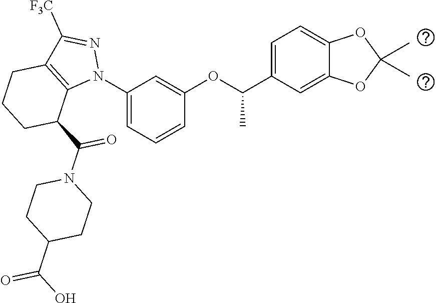 [SMILES: C[C@H](Oc1cccc(-n2nc(C(F)(F)F)c3c2[C@@H](C(=O)N2CCC(C(=O)O)CC2)CCC3)c1)c1ccc2c(c1)OC(C)(C)O2] | 1-[(7S)-1-[3-[(1S)-1-(2,2-difluoro-1,3- benzodioxol-5-yl)ethoxy]phenyl]-3- (trifluoromethyl)-4,5,6,7- tetrahydroindazole-7- carbonyl]piperidine-4-carboxylic acid |
| 120 (P2) | 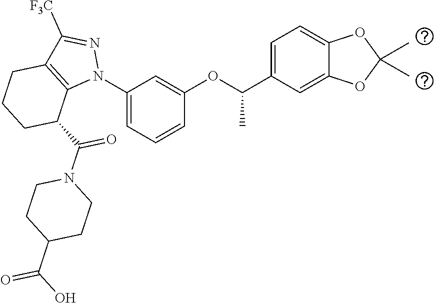 [SMILES: C[C@H](Oc1cccc(-n2nc(C(F)(F)F)c3c2[C@H](C(=O)N2CCC(C(=O)O)CC2)CCC3)c1)c1ccc2c(c1)OC(C)(C)O2] | 1-[(7R)-1-[3-[(1S)-1-(2,2-difluoro-1,3- benzodioxol-5-yl)ethoxy]phenyl]-3- (trifluoromethyl)-4,5,6,7- tetrahydroindazole-7- carbonyl]piperidine-4-carboxylic acid |
| 121 (P1) | 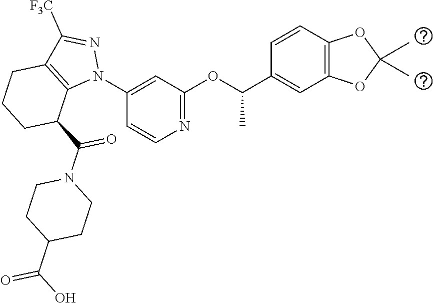 [SMILES: C[C@H](Oc1cc(-n2nc(C(F)(F)F)c3c2[C@@H](C(=O)N2CCC(C(=O)O)CC2)CCC3)ccn1)c1ccc2c(c1)OC(C)(C)O2] | 1-[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazole-7- carbonyl]piperidine-4-carboxylic acid |
| 121 (P2) | 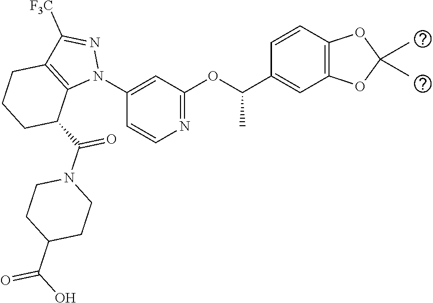 [SMILES: C[C@H](Oc1cc(-n2nc(C(F)(F)F)c3c2[C@H](C(=O)N2CCC(C(=O)O)CC2)CCC3)ccn1)c1ccc2c(c1)OC(C)(C)O2] | 1-[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazole-7- carbonyl]piperidine-4-carboxylic acid |
| 122 (P1) | 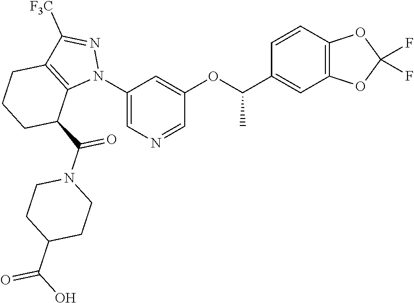 [SMILES: C[C@H](Oc1cncc(-n2nc(C(F)(F)F)c3c2[C@@H](C(=O)N2CCC(C(=O)O)CC2)CCC3)c1)c1ccc2c(c1)OC(F)(F)O2] | 1-[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazole-7- carbonyl]piperidine-4-carboxylic acid |
| 122 (P2) | 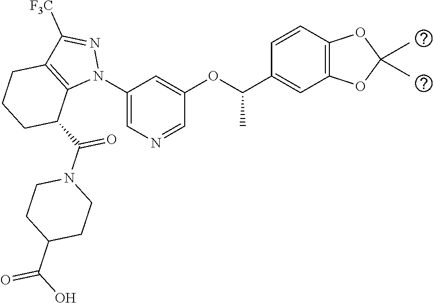 [SMILES: C[C@H](Oc1cncc(-n2nc(C(F)(F)F)c3c2[C@H](C(=O)N2CCC(C(=O)O)CC2)CCC3)c1)c1ccc2c(c1)OC(C)(C)O2] | 1-[(7R)-1-[5-[(1S)-1-(2,2-difluoro-1,3- benzodioxol-5-yl)ethoxy]-3-pyridyl]-3- (trifluoromethyl)-4,5,6,7- tetrahydroindazole-7- carbonyl]piperidine-4-carboxylic acid |
| 123 | 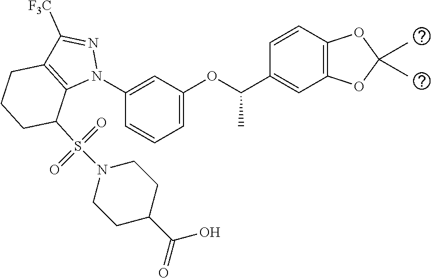 [SMILES: C[C@H](Oc1cccc(-n2nc(C(F)(F)F)c3c2C(S(=O)(=O)N2CCC(C(=O)O)CC2)CCC3)c1)c1ccc2c(c1)OC(C)(C)O2] | 1-[[1-[3-[(1S)-1-(2,2-difluoro-1,3- benzodioxol-5-yl)ethoxy]phenyl]-3- (trifluoromethyl)-4,5,6,7- tetrahydroindazol-7- yl]sulfonyl]piperidine-4-carboxylic acid |
| 124 (P1) | 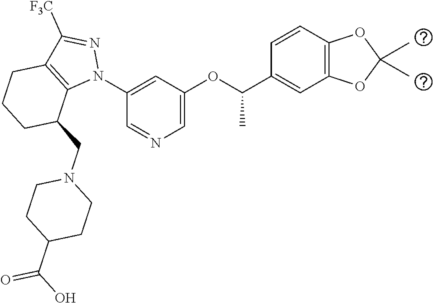 [SMILES: C[C@H](Oc1cncc(-n2nc(C(F)(F)F)c3c2[C@@H](CN2CCC(C(=O)O)CC2)CCC3)c1)c1ccc2c(c1)OC(C)(C)O2] | 1-[[(7R)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl]methyl]piperidine-4-carboxylic acid |
| 124 (P2) | 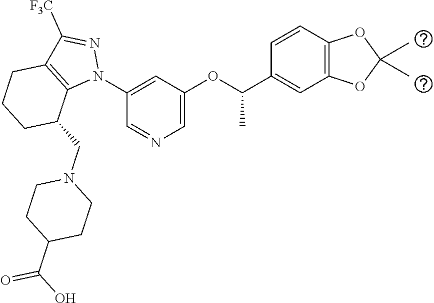 [SMILES: C[C@H](Oc1cncc(-n2nc(C(F)(F)F)c3c2[C@H](CN2CCC(C(=O)O)CC2)CCC3)c1)c1ccc2c(c1)OC(C)(C)O2] | 1-[[(7S)-1-[5-[(1S)-1-(2,2-difluoro-1,3- benzodioxol-5-yl)ethoxy]-3-pyridyl]-3- (trifluoromethyl)-4,5,6,7- tetrahydroindazol-7- yl]methyl]piperidine-4-carboxylic acid |
| 125 (P1) | 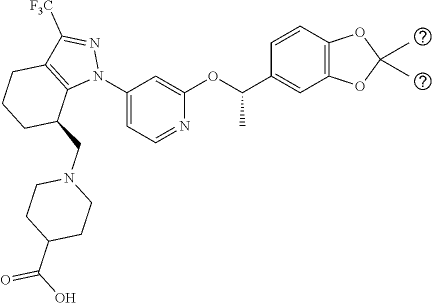 [SMILES: C[C@H](Oc1cc(-n2nc(C(F)(F)F)c3c2[C@@H](CN2CCC(C(=O)O)CC2)CCC3)ccn1)c1ccc2c(c1)OC(C)(C)O2] | 1-[[(7R)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]methyl]piperidine-4-carboxylic acid |
| 125 (P2) | 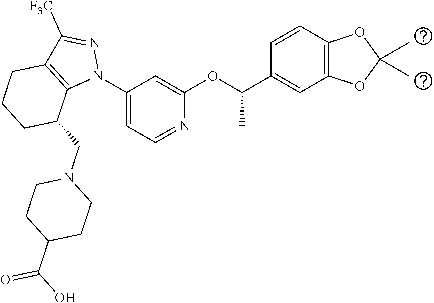 [SMILES: C[C@H](Oc1cc(-n2nc(C(F)(F)F)c3c2[C@H](CN2CCC(C(=O)O)CC2)CCC3)ccn1)c1ccc2c(c1)OC(C)(C)O2] | 1-[(7S)-1-[2-[(1S)-1-(2,2-difluoro-1,3- benzodioxol-5-yl)ethoxy]-4-pyridyl]-3- (trifluoromethyl)-4,5,6,7- tetrahydroindazol-7- yl]methyl]piperidine-4-carboxylic acid |
| 126 | 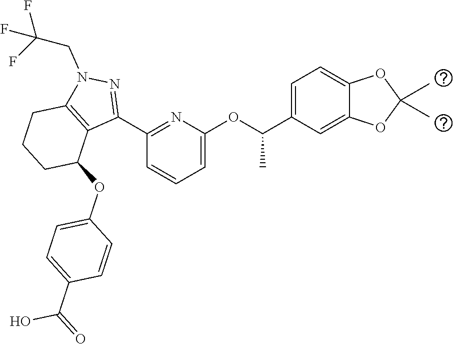 [SMILES: C[C@H](Oc1cccc(-c2nn(CC(F)(F)F)c3c2[C@@H](Oc2ccc(C(=O)O)cc2)CCC3)n1)c1ccc2c(c1)OC(C)(C)O2] | 4-[[(4S)-3-[6-[(1S)-1-(2,2-difluoro-1,3- benzodioxol-5-yl)ethoxy]-2-pyridyl]-1- (2,2,2-trifluoroethyl)-4,5,6,7- tetrahydroindazol-4-yl]oxy]benzoic acid |
| 127 | 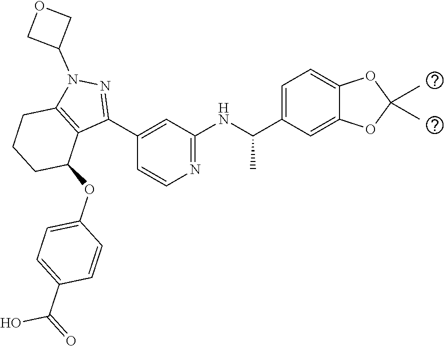 [SMILES: C[C@H](Nc1cc(-c2nn(C3COC3)c3c2[C@@H](Oc2ccc(C(=O)O)cc2)CCC3)ccn1)c1ccc2c(c1)OC(C)(C)O2] | 4-[[(4S)-3-[2-[[(1S)-1-(2,2-difluoro-1,3- benzodioxol-5-yl)ethyl]amino]-4- pyridyl]-1-(oxetan-3-yl)-4,5,6,7- tetrahydroindazol-4-yl]oxy]benzoic acid |
| 128 | 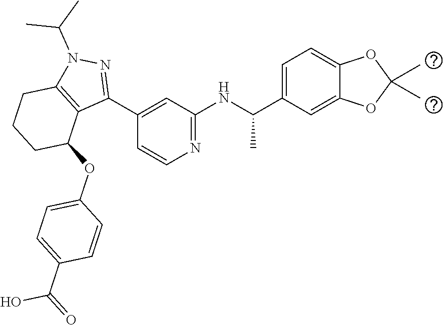 [SMILES: CC(C)n1nc(-c2ccnc(N[C@@H](C)c3ccc4c(c3)OC(C)(C)O4)c2)c2c1CCC[C@@H]2Oc1ccc(C(=O)O)cc1] | 4-[[(4S)-3-[2-[[(1S)-1-(2,2-difluoro-1,3- benzodioxol-5-yl)ethyl]amino]-4- pyridyl]-1-isopropyl-4,5,6,7- tetrahydroindazol-4-yl]oxy]benzoic acid |
| 129 | 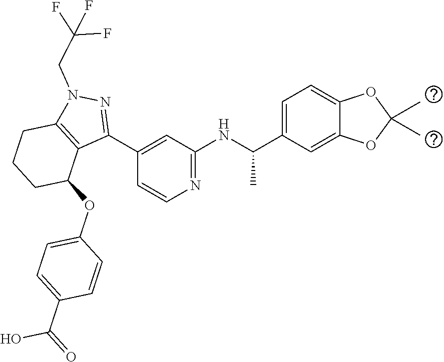 [SMILES: C[C@H](Nc1cc(-c2nn(CC(F)(F)F)c3c2[C@@H](Oc2ccc(C(=O)O)cc2)CCC3)ccn1)c1ccc2c(c1)OC(C)(C)O2] | 4-[[(4S)-3-[2-[[(1S)-1-(2,2-difluoro-1,3- benzodioxol-5-yl)ethyl]amino]-4- pyridyl]-1-(2,2,2-trifluoroethyl)-4,5,6,7- tetrahydroindazol-4-yl]oxy]benzoic acid |

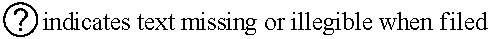

or a pharmaceutically acceptable salt thereof.

49. A pharmaceutical composition comprising a compound of any of claims 1-48 and a pharmaceutically acceptable adjuvant.

50. A method of treating a CFTR-mediated disease or disorder comprising administering a patient in need there of a compound any of claims 1-48 or a pharmaceutical composition of claim 49.

51. The method of claim 50, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

52. The method of claim 50 or 51, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

53. The method of any one of claims 50-52, wherein the disease or condition is cystic fibrosis.

54. A method of treating cystic fibrosis in a subject, comprising administering to the subject a therapeutically effective amount of a compound of any one of claims 1-48 or a pharmaceutical composition of claim 49.

55. The method of claim 54, wherein the subject is human.
